(19) United States
(12) Patent Application Publication
(10) Pub. No.: US 2024/0166646 A1
(43) Pub. Date: May 23, 2024

(54) 1H-PYRROLO[2,3-B]PYRIDINE DERIVATIVES AS BCL-2 INHIBITORS FOR THE TREATMENT OF NEOPLASTIC AND AUTOIMMUNE DISEASES

(71) Applicant: Newave Pharmaceutical Inc., Pleasanton, CA (US)

(72) Inventor: Yi Chen, Pleasanton, CA (US)

(21) Appl. No.: 18/268,632

(22) PCT Filed: Dec. 20, 2021
(86) PCT No.: PCT/US2021/064278

§ 371 Date: Jun. 21, 2023

Related U.S. Application Data
(60) Provisional application No. 63/271,195, filed on Oct. 24, 2021.
(60) Provisional application No. 63/218,461, filed on Jul. 5, 2021.
(60) Provisional application No. 63/139,307, filed on Jan. 20, 2021.

(30) Foreign Application Priority Data
Dec. 22, 2020 (WO) PCT/US2020/066650

(51) Int. Cl. C07D 471/04; A61P 35/02; C07D 498/04; C07D 498/14

(52) U.S. Cl. CPC C07D 471/04; A61P 35/02; C07D 498/04; C07D 498/14

(57) ABSTRACT

The present invention relates to compounds of e.g. formula (I) as BCL-2 inhibitors for the treatment of neoplastic, autoimmune or neurodegenerative diseases. Preferred compounds are e.g. fused 1H-pyrrolo[2,3-b]pyridine derivatives of e.g. formula (II)

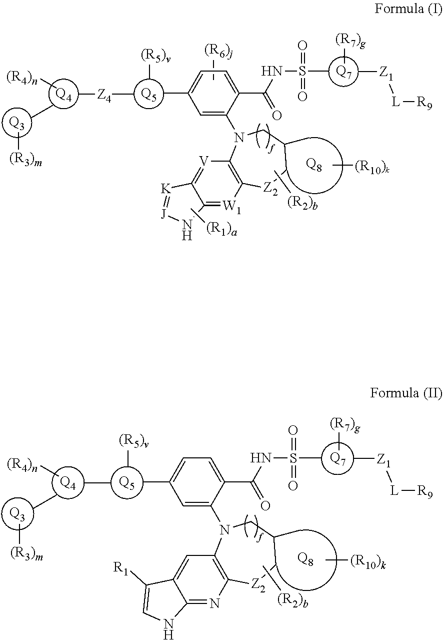

### REFERENCE TO RELATED APPLICATIONS

This international patent application claims priority to International Patent Application No. PCT/US2020/066650, filed on Dec. 22, 2020, and to U.S. Provisional Patent Application Nos. 63/139,307, filed on Jan. 20, 2021; 63/218,461, filed on Jul. 5, 2021; and 63/271,195, filed on Oct. 24, 2021, the entire contents of each of the above-referenced applications are hereby incorporated herein by reference.

### BACKGROUND OF THE INVENTION

Apoptosis, or programmed cell death, is a conserved and regulated process that is the primary mechanism for the removal of aged, damaged and unnecessary cells. The ability to block apoptotic signaling is a key hallmark of cancer and is thus important for oncogenesis, tumor maintenance and chemoresistance [Hanahan, D. & Weinberg, R. A. The hallmarks of cancer. Cell 100, 57-70 (2000).]. Dynamic binding interactions between prodeath (for example, BCL-2-associated X protein (BAX), BCL-2 antagonist/killer 1 (BAK), BCL-2-associated agonist of cell death (BAD), BCL-2-like 11 (BIM), NOXA and BCL-2 binding component 3 (PUMA)) and prosurvival (BCL-2, BCL-XL, BCL-2-like 2 (BCL-W), myeloid cell leukemia sequence 1 (MCL-1) and BCL-2-related protein A1 (BFL-1)) proteins in the BCL-2 family control commitment to programmed cell death. Altering the balance among these opposing factions provides one means by which cancer cells undermine normal apoptosis and gain a survival advantage [Youle, R. J. & Strasser, A. The BCL-2 protein family: opposing activities that mediate cell death. Nat. Rev. Mol. Cell Biol. 9, 47-59 (2008)].

BCL-2, the first identified apoptotic regulator, was originally cloned from the breakpoint of a t(14; 18) translocation present in human B cell lymphomas [Tsujimoto, Y., et al. Science 228, 1440-1443 (1985); Cleary, M. L., et al Cell 47, 19-28 (1986); Boise, L. H. et al. Cell 74, 597-608 (1993)]. This protein has since been shown to have a dominant role in the survival of multiple lymphoid malignancies [Vaux, D. L., et al pre-B cells. Nature 335, 440-442 (1988)]. Overexpression of Bcl-2 proteins correlates with resistance to chemotherapy, clinical outcome, disease progression, overall prognosis or a combination thereof in various cancers and disorders of the immune system. Involvement of Bcl-2 proteins in bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, and the like is described in PCT/US2004/36770, published as WO 2005/049593, and PCT/US2004/37911, published as WO/2005/049594. Involvement of Bcl-2 proteins in immune and autoimmune diseases is described in Current Allergy and Asthma Reports 2003, 3, 378-384; British Journal of Hematology 2000, 110(3), 584-90; Blood 2000, 95(4), 1283-92; and New England Journal of Medicine 2004, 351(14), 1409-1418. Involvement of Bcl-2 proteins in arthritis is disclosed in WO 2009/064938. Involvement of Bcl-2 proteins in bone marrow transplant rejection is disclosed in US 2008-0182845 A1. All incorporated herein by reference.

In the last decade, several Bcl-2 inhibitors such as ABT-737, ABT-263, and ABT-199 as shown below have been identified and entered human clinical trials for cancers treatment.

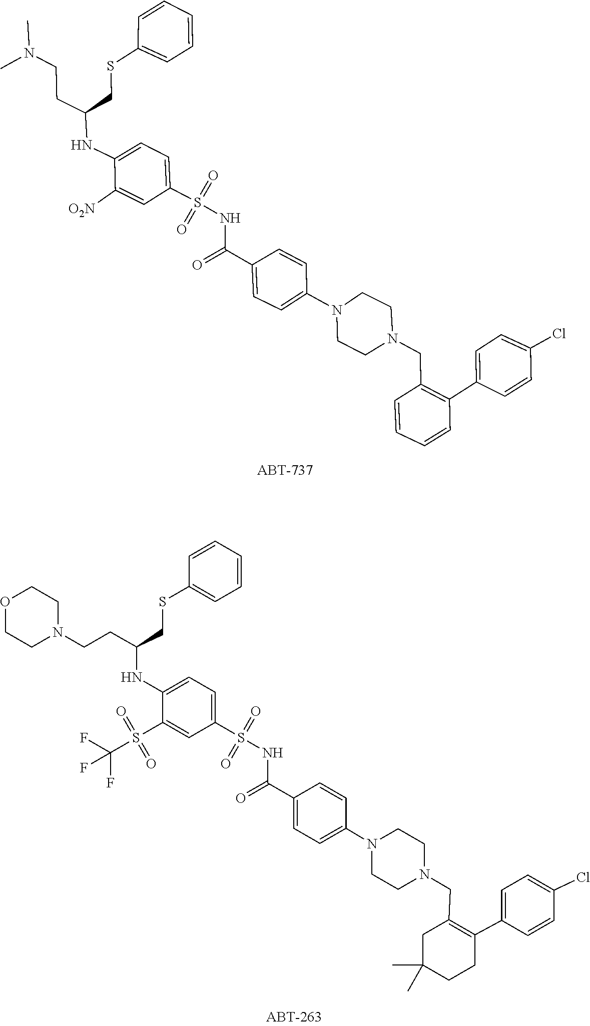 [SMILES: CN(C)CC[C@@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; CC1(C)CCC(c2ccc(Cl)cc2)=C(CN2CCN(c3ccc(C(=O)NS(=O)(=O)c4ccc(N[C@@H](CCN5CCOCC5)CSc5ccccc5)c(S(=O)(=O)C(F)(F)F)c4)cc3)CC2)C1]

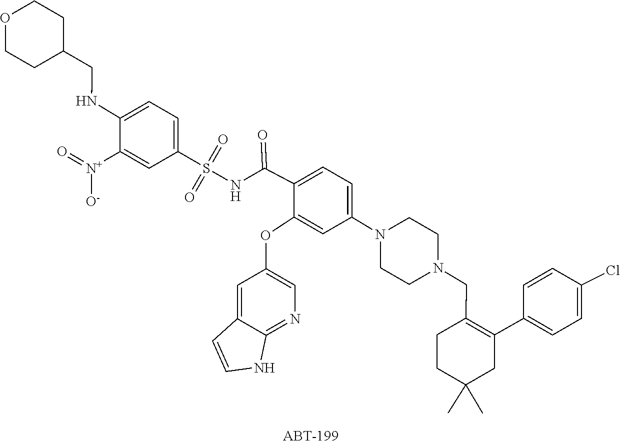 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

ABT-737 is discovered by nuclear magnetic resonance (NMR)-based screening, parallel synthesis and structure based fragment drug design [Tillman Oltersdorf, et al, Nature, Vol 435, 2005, p 677]. ABT-737 a small-molecule inhibitor of the anti-apoptotic proteins Bcl-2, Bcl-XL and Bcl-w, with an affinity two to three orders of magnitude more potent than previously reported compounds. Mechanistic studies reveal that ABT-737 does not directly initiate the apoptotic process, but enhances the effects of death signals, displaying synergistic cytotoxicity with chemotherapeutics and radiation. ABT-737 exhibits single-agent-mechanism-based killing of cells from lymphoma and small-cell lung carcinoma lines, as well as primary patient-derived cells, and in animal models, ABT-737 improves survival, causes regression of established tumors, and produces cures in a high percentage of the mice. Unfortunately, ABT-737 is not orally bioavailable, and its formulation for intravenous delivery is hampered by its low aqueous solubility.

After extensive MedChem effort, an orally bioavailable Bcl-2 inhibitor ABT-263 (Navitoclax) has been developed [Cheol-Min Park, et al J. Med. Chem. 2008, 51, 6902-6915]. ABT-263 is a potent inhibitor of Bcl-xL, Bcl-2 and Bcl-w with Ki of ≤0.5 nM, ≤1 nM and 1 nM. ABT-263 has an IC₅₀ of 110 nM against SCLC H146 cell line. When ABT-263 is administered at 100 mg/kg/day in the H345 xenograft model, significant antitumor efficacy is observed with 80% TGI and 20% of treated tumors indicating at least a 50% reduction in tumor volume. Oral administration of ABT-263 alone causes complete tumor regressions in xenograft models of small-cell lung cancer and acute lymphoblastic leukemia [Tse C, et al. Cancer Res. 2008, 68(9), 3421-3428]. In the clinical trial, however, the inhibition of BCL-XL by ABT-263 (navitoclax) induces a rapid, concentration-dependent decrease in the number of circulating platelets. This mechanism-based thrombocytopenia is the dose-limiting toxicity of single-agent navitoclax treatment in patients and limits the ability to drive drug concentrations into a highly efficacious range.

Thus, a BCL-2 selective (BCL-XL sparing) inhibitor would culminate in substantially reduced thrombocytopenia while maintaining efficacy in lymphoid malignancies. The resulting increase in the therapeutic window should allow for greater BCL-2 suppression and clinical efficacy in BCL-2-dependent tumor types. After extensive MedChem, ABT-199 (GDC-0199) has been successfully developed [Andrew J Souers, et al, Nature Medicine, Volume 19, 22, p202, 2013]. ABT-199 is a Bcl-2-selective inhibitor with Ki of <0.01 nM, >4800-fold more selective versus Bcl-xL and Bcl-w, and no activity to Mcl-1. ABT-199 potently inhibits RS4; 11 cells with EC₅₀ of 8 nM. In addition, ABT-199 induces a rapid apoptosis in RS4; 11 cells with cytochrome c release, caspase activation, and the accumulation of sub-G0/G1 DNA. Quantitative immunoblotting reveals that sensitivity to ABT-199 correlated strongly with the expression of Bcl-2, including NHL, DLBCL, MCL, AML and ALL cell lines. ABT-199 also induces apoptosis in CLL with an average EC₅₀ of 3.0 nM. A single dose of 100 mg/kg of ABT-199 causes a maximal tumor growth inhibition of 95% and tumor growth delay of 152% in RS4; 11 xenografts. ABT-199 also inhibits xenograft growth (DoHH2, Granta-519) as a single agent or in combination with Bendamustine and other agents. Human Phase I and II data showed that ABT-199 is highly efficacious for CLL who have 17p deletion, and was approved by FDA in 2016.

WO/2017/132474, WO/2019/040550, and WO/2019/040573 disclosed a novel class of BCL-2 inhibitors. However, there is still a strong need for continuing search in this field of art for more potent BCL-2 inhibitor.

### SUMMARY OF THE INVENTION

In a first embodiment, this invention provides compounds of the Formula (0) or an N-oxide thereof, or a pharmaceutically acceptable salt, solvate, polymorph, tautomer, stereoisomer, an isotopic form, or a prodrug of said compound of Formula (0) or N-oxide thereof:

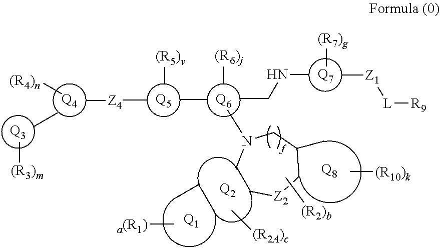

wherein
G is C(O), S(O₂), P(O)(Rₐ), or S(O)(NRₐ);
E is S(O₂), P(O)(Rₐ), S(O)(NRₐ) or C(O);
each of Q₁, Q₂, Q₃, Q₄, Q₅, Q₆, Q₇, and Q₈, independently, is cycloalkyl, cycloalkenyl, heterocycloalkyl, heterocycloalkenyl, spiroheterocyclyl, fused heterocyclyl, bridged heterocyclyl, aryl, or heteroaryl, wherein one or more (e.g., both) border ring atom(s) between ring Q₈ and the ring with Z₂ can be carbon or heteroatom(s);
each of R₁, R₂, R_{2A}, R₃, R₄, R₅, R₆, R₇, R₉, and R₁₀, independently, is H, D, alkyl, spiroalkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiroheterocyclyl, fused heterocyclyl, bridged heterocyclyl, aryl, heteroaryl is optionally substituted with one or more R_{d};
two of R₁ group, taken together with the atom to which they are attached, may optionally form a cycloalkyl, heterocycloalkyl, aryl, or heteroaryl, in which said cycloalkyl, heterocycloalkyl, aryl, or heteroaryl of R₁, is optionally substituted with one or more R_{d};
two of R₂ group, taken together with the atom to which they are attached, may optionally form a cycloalkyl, heterocycloalkyl, aryl, or heteroaryl, in which said cycloalkyl, heterocycloalkyl, aryl, or heteroaryl of R₂, is optionally substituted with one or more R_{d};
two of R₃ group, taken together with the atom to which they are attached, may optionally form a cycloalkyl, heterocycloalkyl, aryl, or heteroaryl, in which said cycloalkyl, heterocycloalkyl, aryl, or heteroaryl of R₃, is optionally substituted with one or more R_{d};
two of R₄ group, taken together with the atom to which they are attached, may optionally form a cycloalkyl, heterocycloalkyl, aryl, or heteroaryl, in which said cycloalkyl, heterocycloalkyl, aryl, or heteroaryl of R₄, is optionally substituted with one or more R_{d};
two of R₅ group, taken together with the atom to which they are attached, may optionally form a cycloalkyl, heterocycloalkyl, aryl, or heteroaryl, in which said cycloalkyl, heterocycloalkyl, aryl, or heteroaryl of R₅, is optionally substituted with one or more R_{d};
two of R₆ group, taken together with the atom to which they are attached, may optionally form a cycloalkyl, heterocycloalkyl, aryl, or heteroaryl, in which said cycloalkyl, heterocycloalkyl, aryl, or heteroaryl of R₆, is optionally substituted with one or more R_{d};
two of R₇ group, taken together with the atom to which they are attached, may optionally form a cycloalkyl, heterocycloalkyl, aryl, or heteroaryl, in which said cycloalkyl, heterocycloalkyl, aryl, or heteroaryl of R₇, is optionally substituted with one or more R_{d};
two of R₁₀ group, taken together with the atom to which they are attached, may optionally form a cycloalkyl, heterocycloalkyl, aryl, or heteroaryl, in which said cycloalkyl, heterocycloalkyl, aryl, or heteroaryl of R₁₀, is optionally substituted with one or more R_{d};
R₃ and R₄ group, taken together with the atom to which they are attached, may optionally form a cycloalkyl, heterocycloalkyl, aryl, or heteroaryl, in which said cycloalkyl, heterocycloalkyl, aryl, or heteroaryl of R₃ or R₄, is optionally substituted with one or more R_{d};
R₄ and R₅ group, taken together with the atom to which they are attached, may optionally form a cycloalkyl, heterocycloalkyl, aryl, or heteroaryl, in which said cycloalkyl, heterocycloalkyl, aryl, or heteroaryl of R₄ or R₅, is optionally substituted with one or more R_{d};
R₅ and R₆ group, taken together with the atom to which they are attached, may optionally form a cycloalkyl, heterocycloalkyl, aryl, or heteroaryl, in which said cycloalkyl, heterocycloalkyl, aryl, or heteroaryl of R₅ or R₆, is optionally substituted with one or more R_{d};
R₇ and —Z₁-L-R₉ group, taken together with the atom to which they are attached, may optionally form a cycloalkyl, cycloalkenyl, heterocycloalkyl, heterocycloalkenyl, aryl, or heteroaryl, in which said cycloalkyl, cycloalkenyl, heterocycloalkyl, heterocycloalkenyl, aryl, or heteroaryl of R₇ and —Z₁-L-R₉, is optionally substituted with one or more R_{d};
each of Z₁, and Z₂, independently, is a bond, (CRₐR_{b})ₚ, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, (CRₐR_{b})ₚC(O)N(Rₐ)(CRₐR_{b})_{q}, a bivalent alkenyl group, or a bivalent alkynyl group;
Z₄ is a bond or an alkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, or heteroaryl, in which said cycloalkyl, cycloalkenyl, heterocycloalkyl, heterocycloalkenyl, spiroheterocyclyl, fused heterocyclyl, bridged heterocyclyl, aryl, or heteroaryl is optionally substituted with one or more R_{d};
L is bond, alkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, or heteroaryl is optionally substituted with one or more R_{d}; each Rₐ, R_{b}, R_{c}, and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, alkoxy, alkoxyalkyl, cycloalkyl, cycloalkenyl, heterocycloalkyl, heterocycloalkenyl, spiroheterocyclyl, fused heterocyclyl, bridged heterocyclyl, aryl, or heteroaryl is optionally substituted with one or more Rₑ;
each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, alkoxy, alkoxyalkyl, cycloalkyl, cycloalkenyl, heterocycloalkyl, heterocycloalkenyl, spiroheterocyclyl, fused heterocyclyl, bridged heterocyclyl, aryl, heteroaryl is optionally substituted with one or more R_{f}; and
each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl;
Rₐ and R_{b} groups, taken together with the atom to which they are attached, may optionally form a cycloalkyl, heterocycloalkyl, aryl, or heteroaryl, in which said cycloalkyl, heterocycloalkyl, aryl, or heteroaryl is optionally substituted with one or more Rₑ;
R_{b} and R_{c} groups, taken together with the atom to which they are attached, may optionally form a cycloalkyl, heterocycloalkyl, aryl, or heteroaryl, in which said cycloalkyl, heterocycloalkyl, aryl, or heteroaryl is optionally substituted with one or more Rₑ;
two of R_{d} groups, taken together with the atom to which they are attached, may optionally form a cycloalkyl, heterocycloalkyl, aryl, or heteroaryl, in which said cycloalkyl, heterocycloalkyl, aryl, or heteroaryl is optionally substituted with one or more Rₑ;
two of Rₑ groups, taken together with the atom to which they are attached, may optionally form a cycloalkyl, heterocycloalkyl, aryl, or heteroaryl, in which said cycloalkyl, heterocycloalkyl, aryl, or heteroaryl is optionally substituted with one or more R_{f}; and
two of R_{f} groups, taken together with the atom to which they are attached, may optionally form a cycloalkyl, heterocycloalkyl, aryl, or heteroaryl, in which said cycloalkyl, heterocycloalkyl, aryl, or heteroaryl is optionally substituted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl; and
each of a, b, c, m, n, j, k, v and g is, independently, 0, 1, 2, 3, 4, 5, 6, 7, or 8;
f is 0, 1, or 2; and
each of p and q is, independently, 0, 1, 2, 3, 4, or 5.

In certain embodiments, the invention provides a compound represented by Formula (I):

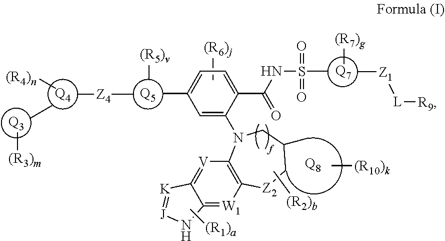

wherein
each of W₁, V, K, J, independently, is C(Rₐ) or N;
each of Q₃, Q₄, Q₅, Q₇, and Q₈, independently, is cycloalkyl, cycloalkenyl, heterocycloalkyl, heterocycloalkenyl, spiroheterocyclyl, fused heterocyclyl, bridged heterocyclyl, aryl, or heteroaryl, wherein one or more (e.g., both) border ring atom(s) between ring Q₈ and the ring with Z₂ can be carbon or heteroatom(s);
each of R₁, R₂, R₃, R₄, R₅, R₆, R₇, R₉, and R₁₀, independently, is H, D, alkyl, spiroalkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiroheterocyclyl, fused heterocyclyl, bridged heterocyclyl, aryl, or heteroaryl is optionally substituted with one or more R_{d}; two of R₁ group, taken together with the atom to which they are attached, may optionally form a cycloalkyl, heterocycloalkyl, aryl, or heteroaryl, in which said cycloalkyl, heterocycloalkyl, aryl, or heteroaryl of R₁, is optionally substituted with one or more R_{d};
two of R₂ group, taken together with the atom to which they are attached, may optionally form a cycloalkyl, heterocycloalkyl, aryl, or heteroaryl, in which said cycloalkyl, heterocycloalkyl, aryl, or heteroaryl of R₂, is optionally substituted with one or more R_{d};
two of R₃ group, taken together with the atom to which they are attached, may optionally form a cycloalkyl, heterocycloalkyl, aryl, or heteroaryl, in which said cycloalkyl, heterocycloalkyl, aryl, or heteroaryl of R₃, is optionally substituted with one or more R_{d};
two of R₄ group, taken together with the atom to which they are attached, may optionally form a cycloalkyl, heterocycloalkyl, aryl, or heteroaryl, in which said cycloalkyl, heterocycloalkyl, aryl, or heteroaryl of R₄, is optionally substituted with one or more R_{d};
two of R₅ group, taken together with the atom to which they are attached, may optionally form a cycloalkyl, heterocycloalkyl, aryl, or heteroaryl, in which said cycloalkyl, heterocycloalkyl, aryl, or heteroaryl of R₅, is optionally substituted with one or more R_{d};
two of R₆ group, taken together with the atom to which they are attached, may optionally form a cycloalkyl, heterocycloalkyl, aryl, or heteroaryl, in which said cycloalkyl, heterocycloalkyl, aryl, or heteroaryl of R₆, is optionally substituted with one or more R_{d};
two of R₇ group, taken together with the atom to which they are attached, may optionally form a cycloalkyl, heterocycloalkyl, aryl, or heteroaryl, in which said cycloalkyl, heterocycloalkyl, aryl, or heteroaryl of R₇, is optionally substituted with one or more R_{d};
two of R₁₀ group, taken together with the atom to which they are attached, may optionally form a cycloalkyl, heterocycloalkyl, aryl, or heteroaryl, in which said cycloalkyl, heterocycloalkyl, aryl, or heteroaryl of R₈, is optionally substituted with one or more R_{d};
R₃ and R₄ group, taken together with the atom to which they are attached, may optionally form a cycloalkyl, heterocycloalkyl, aryl, or heteroaryl, in which said cycloalkyl, heterocycloalkyl, aryl, or heteroaryl of R₃ or R₄, is optionally substituted with one or more R_{d};
R₄ and R₅ group, taken together with the atom to which they are attached, may optionally form a cycloalkyl, heterocycloalkyl, aryl, or heteroaryl, in which said cycloalkyl, heterocycloalkyl, aryl, or heteroaryl of R₄ or R₅, is optionally substituted with one or more R_{d};
R₅ and R₆ group, taken together with the atom to which they are attached, may optionally form a cycloalkyl, heterocycloalkyl, aryl, or heteroaryl, in which said cycloalkyl, heterocycloalkyl, aryl, or heteroaryl of R₅ or R₆, is optionally substituted with one or more R_{d};
R₂ and R₁₀ group, taken together with the atom to which they are attached, may optionally form a cycloalkyl, heterocycloalkyl, aryl, or heteroaryl, in which said cycloalkyl, heterocycloalkyl, aryl, or heteroaryl of R₂ and R₁₀, is optionally substituted with one or more R_{d};
R₇ and —Z₁-L-R₉ group, taken together with the atom to which they are attached, may optionally form a cycloalkyl, cycloalkenyl, heterocycloalkyl, heterocycloalkenyl, spiroheterocyclyl, fused heterocyclyl, bridged heterocyclyl, aryl, or heteroaryl, in which said cycloalkyl, cycloalkenyl, heterocycloalkyl, heterocycloalkenyl, spiroheterocyclyl, fused heterocyclyl, bridged heterocyclyl, aryl, or heteroaryl of R₇, is optionally substituted with one or more R_{d};
each of Z₁, and Z₂, independently, is a bond, (CRₐR_{b})ₚ, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, (CRₐR_{b})ₚC(O)N(Rₐ)(CRₐR_{b})_{q}, a bivalent alkenyl group, or a bivalent alkynyl group;
Z₄ is a bond or a cycloalkyl, cycloalkenyl, heterocycloalkyl, heterocycloalkenyl, spiroheterocyclyl, fused heterocyclyl, bridged heterocyclyl, aryl, or heteroaryl, in which said cycloalkyl, cycloalkenyl, heterocycloalkyl, heterocycloalkenyl, spiroheterocyclyl, fused heterocyclyl, bridged heterocyclyl, aryl, or heteroaryl is optionally substituted with one or more R_{d};
L is bond, alkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, or heteroaryl is optionally substituted with one or more R_{d};
each of Rₐ, R_{b}, R_{c}, and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, alkoxy, alkoxyalkyl, cycloalkyl, cycloalkenyl, heterocycloalkyl, heterocycloalkenyl, spiroheterocyclyl, fused heterocyclyl, bridged heterocyclyl, aryl, heteroaryl is optionally substituted with one or more Rₑ;
each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, alkoxy, alkoxyalkyl, cycloalkyl, cycloalkenyl, heterocycloalkyl, heterocycloalkenyl, spiroheterocyclyl, fused heterocyclyl, bridged heterocyclyl, aryl, heteroaryl is optionally substituted with one or more R_{f}; and,
each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl;
Rₐ and R_{b}, groups, taken together with the atom to which they are attached, may optionally form a cycloalkyl, heterocycloalkyl, aryl, or heteroaryl, in which said cycloalkyl, heterocycloalkyl, aryl, or heteroaryl is optionally substituted with one or more Rₑ;
R_{b} and R_{c} groups, taken together with the atom to which they are attached, may optionally form a cycloalkyl, heterocycloalkyl, aryl, or heteroaryl, in which said cycloalkyl, heterocycloalkyl, aryl, or heteroaryl of R_{b} and R_{c}, is optionally substituted with one or more Rₑ;
two of R_{d} group, taken together with the atom to which they are attached, may optionally form a cycloalkyl, heterocycloalkyl, aryl, or heteroaryl, in which said cycloalkyl, heterocycloalkyl, aryl, or heteroaryl of R_{d}, is optionally substituted with one or more Rₑ;
two of Rₑ groups, taken together with the atom to which they are attached, may optionally form a cycloalkyl, heterocycloalkyl, aryl, or heteroaryl, in which said cycloalkyl, heterocycloalkyl, aryl, or heteroaryl of Rₑ is optionally substituted with one or more R_{f}; and
two of R_{f} groups, taken together with the atom to which they are attached, may optionally form a cycloalkyl, heterocycloalkyl, aryl, or heteroaryl, in which said cycloalkyl, heterocycloalkyl, aryl, or heteroaryl of R_{f} is optionally substituted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl; and
each of a, b, m, n, j, k, v and g is, independently, 0, 1, 2, 3, 4, 5, 6, 7, or 8;
f is 0, 1, or 2; and
each of p, and q is, independently, 0, 1, 2, 3, 4, or 5.

In certain embodiments, the invention provides a compound represented by Formula (I):

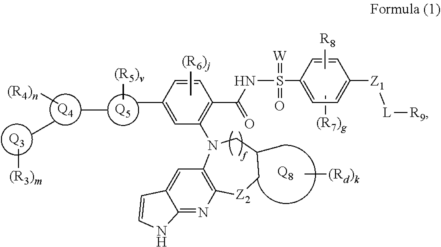

wherein
Q₃ is cycloalkyl, cycloalkenyl, bridged cycloalkyl, heterocycloalkyl, heterocycloalkenyl, spiroheterocyclyl, fused heterocyclyl, bridged heterocyclyl, aryl, or heteroaryl;
Q₄ is cycloalkyl, cycloalkenyl, heterocycloalkyl, heterocycloalkenyl, spiroheterocyclyl, fused heterocyclyl, bridged heterocyclyl, aryl, or heteroaryl;
Q₅ is cycloalkyl, cycloalkenyl, heterocycloalkyl, heterocycloalkenyl, aryl, heteroaryl, spiroheterocyclyl, fused heterocyclyl, or bridged heterocyclyl;
Q₈ is cycloalkyl, cycloalkenyl, heterocycloalkyl, heterocycloalkenyl, aryl, heteroaryl, or spiroheterocyclyl, fused heterocyclyl, or bridged heterocyclyl, wherein each of the broader atoms of Q₈ shared with the ring comprising Z₂ can be a carbon or a heteroatom;
each of R₃, R₄, R₅, R₆, R₇, R₈, and R₉, independently, is H, D, alkyl, spiroalkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiroheterocyclyl, fused heterocyclyl, bridged heterocyclyl, aryl, heteroaryl is optionally substituted with one or more R_{d};
each Rₐ, R_{b}, Rₑ and R_{d}, independently, is H, D, alkyl, spiroalkyl, alkenyl, alkynyl, halo, cyano, amine, nitro, hydroxy, ═O, C(O)NHOH, C(O)OH, C(O)NH₂, alkyl-Rₑ, ORₑ, SRₑ, NH(CH₂)ₚRₑ, C(O)Rₑ, S(O)Rₑ, SO₂Rₑ, C(O)ORₑ, OC(O)Rₑ, NRₑR_{f}, C(O)N(Rₑ)R_{f}, N(Rₑ)C(O)R_{f}, —P(O)RₑR_{f}, -alkyl-P(O)RₑR_{f}, SO₂N(Rₑ)R_{f}, N(R_{b})SO₂R_{c}, —P(O)RₑR_{f}, -alkyl-P(O)RₑR_{f}, —S(O)(═N(Rₑ))R_{f}, —N═S(O)RₑR_{f}, ═NRₑ, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, cycloalkyl, cycloalkenyl, heterocycloalkyl, heterocycloalkenyl, spiroheterocyclyl, fused heterocyclyl, bridged heterocyclyl, aryl, heteroaryl is optionally substituted with one or more Rₑ;
each Rₑ and R_{f}, independently, is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl;
W is O or N(Rₐ);
each of Z₁ and Z₂, independently, is a bond, (CH₂)ₚ, N(Rₐ), O, S, C(O), S(O₂), —O(CH₂)ₚ—, —N(Rₐ)(CH₂)ₚ—, —OC(O)—, —C(O)O—, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, OC(O)N(H)(CH₂)ₚ+₁N(H)(CH₂)_{q}, a bivalent alkenyl group, or a bivalent alkynyl group;
L is bond, alkenyl, alkynyl, cycloalkyl, cycloalkenyl, heterocycloalkyl, heterocycloalkenyl, aryl, or heteroaryl, in which said alkenyl, alkynyl, cycloalkyl, cycloalkenyl, heterocycloalkyl, heterocycloalkenyl, aryl, or heteroaryl is optionally substituted with one or more R_{d};
two of R₃ group, taken together with the atom to which they are attached, may optionally form a cycloalkyl, heterocycloalkyl, aryl, or heteroaryl, in which said cycloalkyl, heterocycloalkyl, aryl, or heteroaryl of R₃, is optionally substituted with one or more R_{d};
two of R₄ group, taken together with the atom to which they are attached, may optionally form a cycloalkyl, heterocycloalkyl, aryl, or heteroaryl, in which said cycloalkyl, heterocycloalkyl, aryl, or heteroaryl of R₄, is optionally substituted with one or more R_{d};
two of R₅ group, taken together with the atom to which they are attached, may optionally form a cycloalkyl, heterocycloalkyl, aryl, or heteroaryl, in which said cycloalkyl, heterocycloalkyl, aryl, or heteroaryl of R₅, is optionally substituted with one or more R_{d};
two of R₆ group, taken together with the atom to which they are attached, may optionally form a cycloalkyl, heterocycloalkyl, aryl, or heteroaryl, in which said cycloalkyl, heterocycloalkyl, aryl, or heteroaryl of R₅, is optionally substituted with one or more R_{d};
R₈ and one R₇ group, or two R₇ groups, taken together with the atom to which they are attached, may optionally form a cycloalkyl, heterocycloalkyl, aryl, or heteroaryl, in which said cycloalkyl, heterocycloalkyl, aryl, or heteroaryl of two R₇ groups or of R₇ and R₈, is optionally substituted with one or more R_{d};
R₇ and —Z₁-L-R₉ group, taken together with the atom to which they are attached, may optionally form a cycloalkyl, cycloalkenyl, heterocycloalkyl, heterocycloalkenyl, aryl, or heteroaryl, in which said cycloalkyl, cycloalkenyl, heterocycloalkyl, heterocycloalkenyl, aryl, or heteroaryl of R₇ and —Z₁-L-R₉, is optionally substituted with one or more R_{d};
R_{b} and R_{c} group, taken together with the atom to which they are attached, may optionally form a cycloalkyl, heterocycloalkyl, aryl, or heteroaryl, in which said cycloalkyl, heterocycloalkyl, aryl, or heteroaryl of R_{b} and R_{c}, is optionally substituted with one or more Rₑ;
two of R_{d} group, taken together with the atom to which they are attached, may optionally form a cycloalkyl, heterocycloalkyl, aryl, or heteroaryl, in which said cycloalkyl, heterocycloalkyl, aryl, or heteroaryl of R_{d}, is optionally substituted with one or more Rₑ;
two of Rₑ group, taken together with the atom to which they are attached, may optionally form a cycloalkyl, heterocycloalkyl, aryl, or heteroaryl, in which said cycloalkyl, heterocycloalkyl, aryl, or heteroaryl of Rₑ is optionally substituted with one or more groups selected from H, D, alkyl, alkenyl, alkynyl, halo, cyano, amine, nitro, hydroxy, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, aryl, or heteroaryl;
each of m, n, j, k, v and g is, independently, 0, 1, 2, 3, 4, 5, 6, 7, or 8;
f is 0, 1, or 2; and
each of p, and q is, independently, 0, 1, 2, 3, 4, or 5.

In certain embodiments, the invention provides a compound represented by Formula (II):

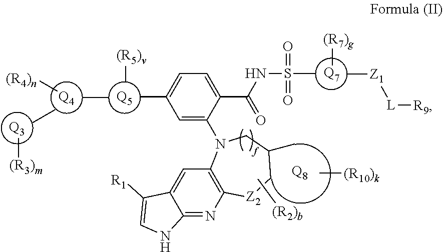

wherein the groups are as defined in Formula (I).

In certain embodiments, the invention provides a compound represented by Formula (2):

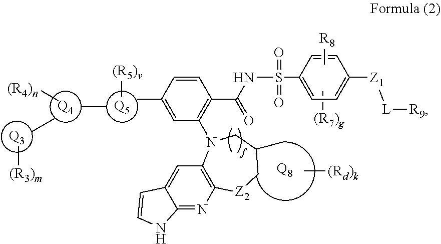

wherein Q4 is cycloalkyl, cycloalkenyl, heterocycloalkyl, heterocycloalkenyl, spiroheterocyclyl; Q5 is cycloalkyl, cycloalkenyl, heterocycloalkyl, heterocycloalkenyl, spiroheterocyclyl; and the remaining groups are as defined in Formula (I).

In certain embodiments, the invention provides a compound represented by Formula (III):

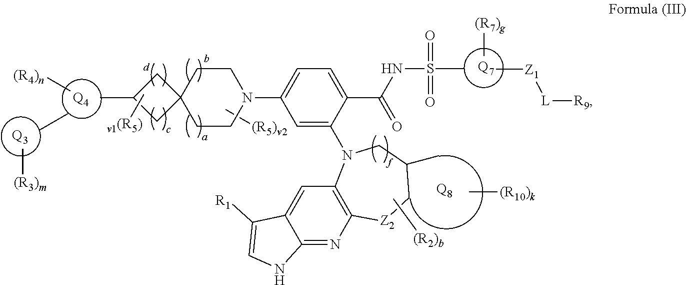

wherein each of a, b, c, and d, independently, is 0, 1, or 2; wherein v1 and v2 are both integers including 0, and v1+v2=v; and the remaining groups are as defined in Formula (I).

In certain embodiments, the invention provides a compound represented by Formula (3):

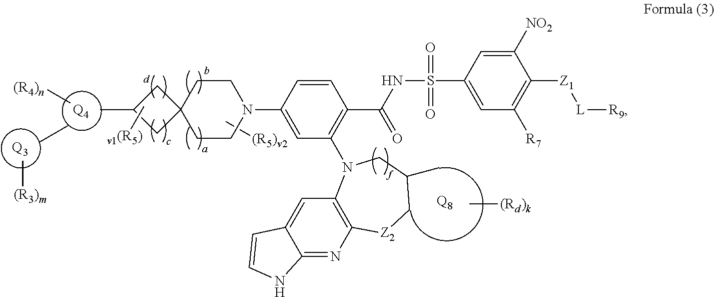

wherein each of a, b, c, and d, independently, is 0, 1, or 2; v1 and v2 are both integers including 0, and v1+v2=v; and the remaining groups are as defined in Formula (2).

In certain embodiments, the invention provides a compound represented by Formula (IV):

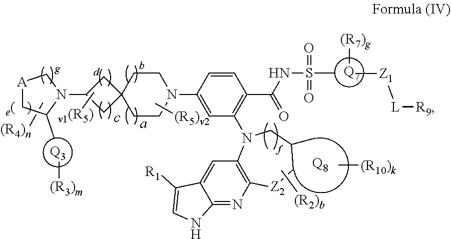

wherein each of e and g, independently is 0, 1, or 2; A is O, S, SO₂, N(Rₐ), or C(RₐR_{b}); and the remaining groups are as defined in Formula (III).

In certain embodiments, the invention provides a compound represented by Formula (IV), wherein R₃ and R₄ group, taken together with the atom to which they are attached, form a cycloalkyl or heterocycloalkyl, in which said cycloalkyl or heterocycloalkyl of R₃ or R₄, is optionally substituted with one or more R_{d}.

In certain embodiments, the invention provides a compound represented by Formula (IV-A):

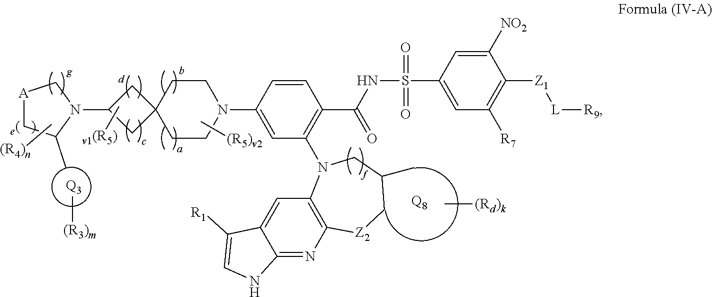

wherein the groups are as defined in Formula (IV).

In certain embodiments, the invention provides a compound represented by Formula (IV-B):

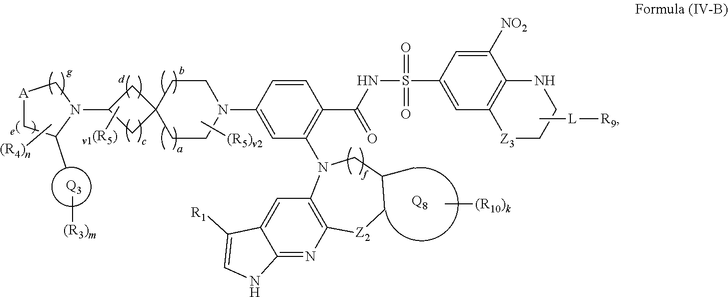

wherein Z₃ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, or OC(O)N(H)(CH₂)ₚ+₁N(H)(CH₂)_{q},

and the remaining groups are as defined in Formula (IV).

In certain embodiments, the invention provides a compound represented by Formula (V):

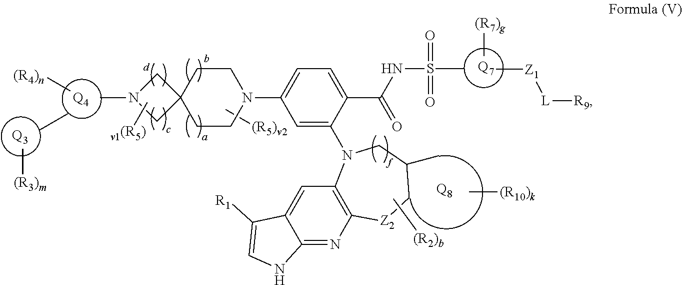

wherein each of a, b, c, and d, independently, is 0, 1, or 2; wherein v1 and v2 are both integers including 0, and v1+v2=v; and the remaining groups are as defined in Formula (I).

In certain embodiments, the invention provides a compound represented by Formula (VI):

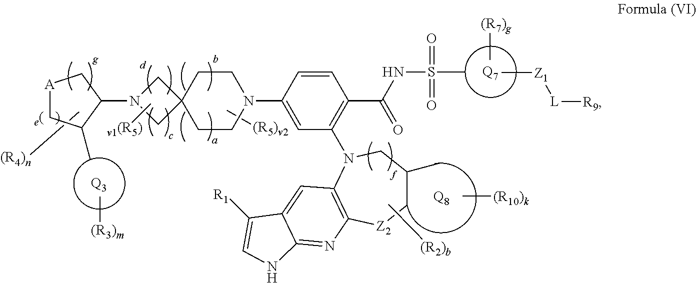

wherein each of e and g, independently is 0, 1, or 2; A is O, S, SO₂, N(Rₐ), or C(RₐR_{b}); and the remaining groups are as defined in Formula (V).

In certain embodiments, the invention provides a compound represented by Formula (VI), wherein R₃ and R₄ group, taken together with the atom to which they are attached, form a cycloalkyl or heterocycloalkyl, in which said cycloalkyl or heterocycloalkyl of R₃ or R₄, is optionally substituted with one or more R_{d}.

In certain embodiments, the invention provides a compound represented by Formula (VII):

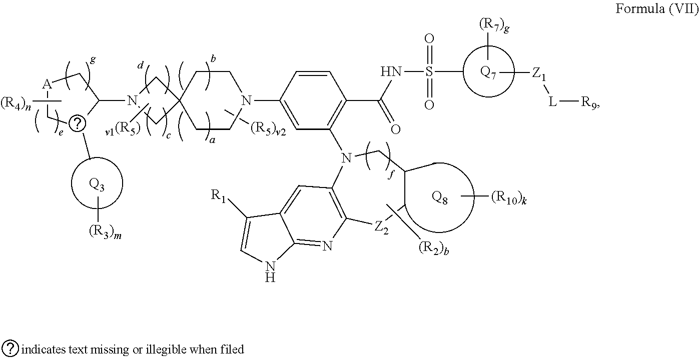

wherein each of e and g, independently is 0, 1, or 2; A is O, S, SO₂, N(Rₐ), or C(RₐR_{b}); and the remaining groups are as defined in Formula (V).

In certain embodiments, the invention provides a compound represented by Formula (A):

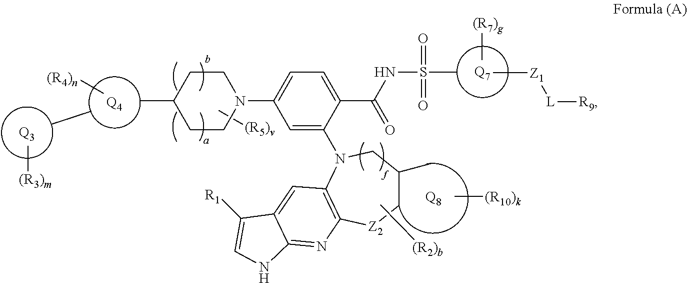

wherein each of a and b, independently, is 0, 1, or 2; and the remaining groups are as defined in Formula (I).

In certain embodiments, the invention provides a compound represented by Formula (B):

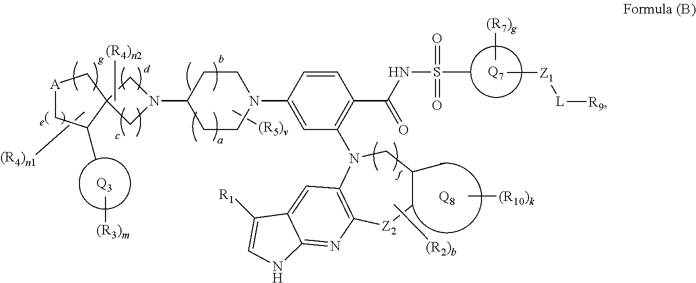

wherein each of a, b, c, d, e and g, independently is 0, 1, or 2; A is O, S, SO₂, N(Rₐ), or C(RₐR_{b}); n1 and n2 are both integers including 0, and n1+n2=n; and the remaining groups are as defined in Formula (II).

In certain embodiments, the invention provides a compound represented by Formula(B), wherein R₃ and R₄ group, taken together with the atom to which they are attached, form a cycloalkyl or heterocycloalkyl, in which said cycloalkyl or heterocycloalkyl of R₃ or R₄, is optionally substituted with one or more R_{d}.

In certain embodiments, the invention provides a compound represented by Formula (C):

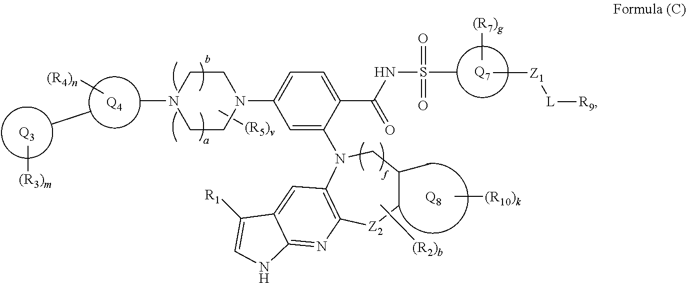

wherein each of a and b, independently, is 0, 1, or 2; and the remaining groups are as defined in Formula (III).

In certain embodiments, the invention provides a compound represented by Formula (D):

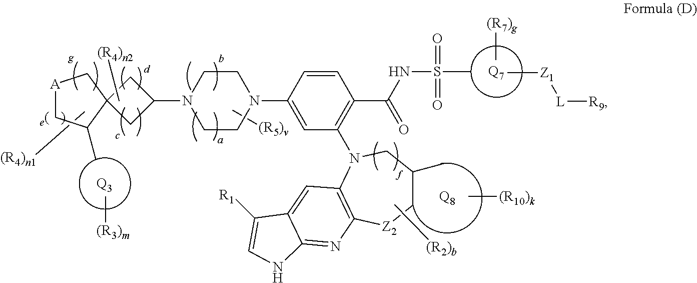

wherein each of a, b, c, d, e and g, independently is 0, 1, or 2; A is O, S, SO₂, N(Rₐ), or C(RₐR_{b}); n1 and n2 are both integers including 0, and n1+n2=n; and the remaining groups are as defined in Formula (IV).

In certain embodiments, the invention provides a compound represented by Formula (D), wherein R₃ and R₄ group, taken together with the atom to which they are attached, form a cycloalkyl or heterocycloalkyl, in which said cycloalkyl or heterocycloalkyl of R₃ or R₄, is optionally substituted with one or more R_{d}.

In certain embodiments, the invention provides a compound represented by Formula (E):

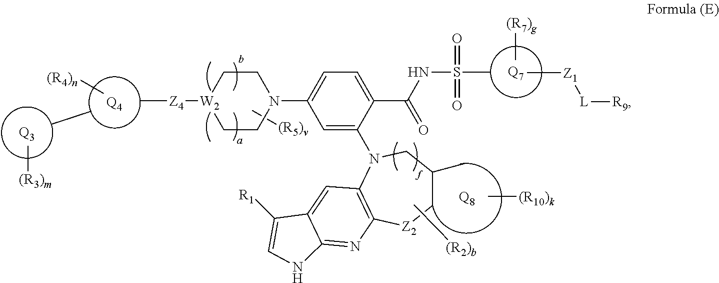

wherein
each of a and b, independently, is 0, 1, or 2;
W₂ is N or C(Rₐ); and
Z₄ is a cycloalkyl, cycloalkenyl, heterocycloalkyl, heterocycloalkenyl, spiroheterocyclyl, fused heterocyclyl, bridged heterocyclyl, aryl, or heteroaryl, in which said cycloalkyl, cycloalkenyl, heterocycloalkyl, heterocycloalkenyl, aryl, or heteroaryl is optionally substituted with one or more R_{d};
and the remaining groups are as defined in Formula (II).

In certain embodiments, the invention provides a compound represented by Formula(E), wherein R₃ and R₄ group, taken together with the atom to which they are attached, form a cycloalkyl or heterocycloalkyl, in which said cycloalkyl or heterocycloalkyl of R₃ or R₄, is optionally substituted with one or more R_{d}.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

Also within the scope of this invention is a pharmaceutical composition containing one or more of the compounds (such as any one of those in Formulae (0), (I)-(VII) including (IV-A) and (IV-B), (A)-(E), and (1)-(3), or a pharmaceutically acceptable salt, solvate, polymorph, tautomer, stereoisomer, an isotopic form, or a prodrug thereof or an N-oxide thereof), modifications, and/or salts thereof described herein, and a pharmaceutically acceptable diluent or carrier, for use in treating a neoplastic disease, therapeutic uses thereof, and use of the compounds for the manufacture of a medicament for treating the disease/disorder.

This invention also relates to a method of treating a neoplastic disease, an autoimmune disease, or a neorodegenerative disease, comprising administering to a subject in need thereof an effective amount of one or more compounds of the invention (such as any one of those in (0), (I)-(VII) including (IV-A) and (IV-B), (A)-(E), and (1)-(3), or a pharmaceutically acceptable salt, solvate, polymorph, tautomer, stereoisomer, an isotopic form, or a prodrug thereof or an N-oxide thereof), modifications, and/or salts thereof described herein, or a pharmaceutical composition comprising the compound(s) of the invention.

In certain embodiments, the neoplastic disease, autoimmune disease, or neorodegenerative disease is characterized by abnormal (e.g., enhanced or increased) Bcl-2 activity. For example, the neoplastic disease can be a hematological malignancy or cancer including solid tumor; the autoimmune disease can be type I diabetes; and the neorodegenerative disease can be schizophrenia.

In certain embodiments, the neoplastic disease is myeloma, multiple myeloma, lymphoma, follicular lymphoma (FL), non-Hodgkin's lymphoma, leukemia, acute leukemia, acute lymphoblastic leukemia (ALL) (such as BCL-2-dependent ALL and pediatric ALL), chronic lymphoblastic leukemia (CLL) (such as relapsed/refractory CLL, del(17p) CLL), chronic myeloid leukemia (CML) (such as blast-crisis CML), mantle cell lymphoma (MCL), diffuse large B-cell lymphoma, lung cancer such as small cell lung cancer (SCLC), melanoma, breast cancer, or prostate cancer, including drug-resistant cancer thereof.

In certain embodiments, the method further comprises administering one or more further treatment(s) effective to treat the neoplastic disease, such as surgery, radiation therapy, a chemotherapeutic agent (such as bendamustine, NL-101 (7-(5-(bis(2-chloroethyl)amino)-1-methyl-1H-benzo[d]imidazol-2-yl)-N-hydroxyheptanamide), cisplatin, carboplatin, etoposide, topotecan), a target thearpy (e.g., an anti-CD20 antibody such as rituximab, a Bruton's tyrosine kinase inhibitor such as ibrutinib and acalabrutinib (ACP-196), a PI3Kδ inhibitor such as idelalisib); an antibody-drug conjugate or ADC (such as anti-CD30 ADC brentuximab vedotin), an immunotherapy (such as an anti-PD-1 antibody including pembrolizumab and nivolumab, or an anti-PD-L1 antibody including atezolizumab, durvalumab, and avelumab), or a CAR-T thearpy (such as tisagenlecleucel, axicabtagene ciloleucel).

Also provided herein is the use of one or more compounds of the invention, or a pharmaceutically acceptable salt thereof, or a pharmaceutical composition comprising one or more compounds of the invention, for the preparation of a medicament for the treatment of the above-referenced diseases or conditions.

In another embodiment, provided herein the compounds of the invention, or a pharmaceutically acceptable salt thereof, or a pharmaceutical composition comprising one or more of the disclosed compounds are for use in treating the above-referenced diseases or conditions.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-4-(2-((S)-2-(2-(prop-1-en-2-yl)phenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(2-vinylphenyl)pyrrolidin-1-yl)-7-azaspiro[3.5]nonan-7-yl)benzamide,
4-(2-((S)-2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-ethynylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(trifluoromethyl)phenyl)pyrrolidin-1-yl)-7-azaspiro[3.5]nonan-7-yl)benzamide,
4-(2-((S)-2-(2-(tert-butyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(2-methylpent-3-yn-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(1,1,1-trifluoro-2-methylpropan-2-yl)phenyl)pyrrolidin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(trifluoromethyl)phenyl)pyrrolidin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(2-vinylphenyl)pyrrolidin-1-yl)-7-azaspiro[3.5]nonan-7-yl)benzamide,
4-(2-((S)-2-(2-ethynylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(tert-butyl)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(1,1,1-trifluoro-2-methylpropan-2-yl)phenyl)pyrrolidin-1-yl)-7-azaspiro[3.5]nonan-7-yl)benzamide,
4-(2-((S)-2-(2-cyclobutylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((1s,4s)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(trifluoromethyl)phenyl)pyrrolidin-1-yl)-7-azaspiro[3.5]nonan-7-yl)benzamide,
N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(2-vinylphenyl)pyrrolidin-1-yl)-7-azaspiro[3.5]nonan-7-yl)benzamide,
4-(2-((S)-2-(2-ethynylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(tert-butyl)phenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(1,1,1-trifluoro-2-methylpropan-2-yl)phenyl)pyrrolidin-1-yl)-7-azaspiro[3.5]nonan-7-yl)benzamide,
4-(2-((S)-2-(2-cyclobutylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1s,4s)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-isopropoxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-4-isopropoxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-(((4-fluorotetrahydro-2H-pyran-4-yl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((S)-1,4-dioxan-2-yl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((S)-1,4-dioxan-2-yl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((S)-1,4-dioxan-2-yl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-3-((1r,4R)-4-methoxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-3-((1s,4S)-4-methoxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-3-((1r,4S)-4-methoxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-3-((1s,4R)-4-methoxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-(((3S)-3-((5R)-5-methyl-4-(oxetan-3-yl)morpholin-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-(((3R)-3-((5R)-5-methyl-4-(oxetan-3-yl)morpholin-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-3-(morpholinomethyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-3-(morpholinomethyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((4,4-difluoropiperidin-1-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((4,4-difluoropiperidin-1-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-3-((4-methylpiperazin-1-yl)methyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-3-((4-methylpiperazin-1-yl)methyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(isopropoxymethyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(isopropoxymethyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-5-nitro-3-(pyridin-2-ylmethyl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-5-nitro-3-(pyridin-2-ylmethyl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-(((4-fluorotetrahydro-2H-pyran-4-yl)methyl)amino)-3-nitrophenyl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((S)-1,4-dioxan-2-yl)methyl)amino)-3-nitrophenyl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-((4-((((1s,4s)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-5-nitro-3-(tetrahydro-2H-pyran-4-yl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-5-nitro-3-(tetrahydro-2H-pyran-4-yl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((S)-1,4-dioxan-2-yl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((S)-1,4-dioxan-2-yl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-4-hydroxycyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-4-hydroxycyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1s,4R)-4-hydroxycyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxycyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide
4-(6-((S)-2-(2-isopropylphenyl)pyrrolidin-1-yl)-2-azaspiro[3.3]heptan-2-yl)-N—(((R)-3-((1r,4R)-4-methoxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-3-((1s,4S)-4-methoxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-3-((1r,4S)-4-methoxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-3-((1s,4R)-4-methoxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-(((3S)-3-((5R)-5-methyl-4-(oxetan-3-yl)morpholin-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-(((3R)-3-((5R)-5-methyl-4-(oxetan-3-yl)morpholin-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-3-(morpholinomethyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-3-(morpholinomethyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((4,4-difluoropiperidin-1-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((4,4-difluoropiperidin-1-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-3-((4-methylpiperazin-1-yl)methyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-3-((4-methylpiperazin-1-yl)methyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide
N—(((R)-3-(isopropoxymethyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(isopropoxymethyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-5-nitro-3-(pyridin-2-ylmethyl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-5-nitro-3-(pyridin-2-ylmethyl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N-((4-(((4-fluorotetrahydro-2H-pyran-4-yl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N-((4-((((S)-1,4-dioxan-2-yl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N-((4-((((1r,4r)-4-methoxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin- 1-yl)-2-azaspiro[3.3]heptan-2-yl)benzamide,
N—(((S)-3-((S)-1,4-dioxan-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N—(((R)-3-((1s,4S)-4-hydroxycyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin- 1-yl)-2-azaspiro[3.3]heptan-2-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N—(((R)-3-((1r,4R)-4-methoxy-4-methylcyclohexyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N-(((3S)-3-((5R)-5-methyl-4-(oxetan-3-yl)morpholin-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N—(((R)-3-(morpholinomethyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N—(((R)-3-((4,4-difluoropiperidin-1-yl)methyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o- tolyl)pyrrolidin-1-yl)-2-azaspiro[3.3]heptan-2-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N—(((R)-3-((4-methylpiperazin-1-yl)methyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin- 1-yl)-2-azaspiro[3.3]heptan-2-yl)benzamide,
N—(((R)-3-(isopropoxymethyl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N—(((R)-5-nitro-3-(pyridin-2-ylmethyl)-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N-((4-((((1r,4r)-4-hydroxy-4-methylcyclohexyl)methyl)amino)-3-nitrophenyl)sulfonyl)-4-(6-((S)-2-(2-(prop-1-en-2-yl)phenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-ethylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-cyclopropylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(2-vinylphenyl)pyrrolidin-1-yl)-2-azaspiro[3.3]heptan-2-yl)benzamide,
4-(6-((S)-2-(2-ethynylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(2-(trifluoromethyl)phenyl)pyrrolidin-1-yl)-2-azaspiro[3.3]heptan-2-yl)benzamide,
4-(6-((S)-2-(2-cyclobutylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-(tert-butyl)phenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(2-(1,1,1-trifluoro-2-methylpropan-2-yl)phenyl)pyrrolidin-1-yl)-2-azaspiro[3.3]heptan-2-yl)benzamide,
N-((4-((((1s,4s)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(o-tolyl)pyrrolidin-1-yl)-2-azaspiro[3.3]heptan-2-yl)benzamide,
N-((4-((((1s,4s)-4-hydroxy-4-methylcyclohexyl)methyl)amino)-3-nitrophenyl)sulfonyl)-4-(6-((S)-2-(2-(prop-1-en-2-yl)phenyl)pyrrolidin-1-yl)-2-azaspiro[3.3]heptan-2-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-cyclopropylphenyl)pyrrolidin-1-yl)-2-azaspiro[3.3]heptan-2-yl)-N-((4-((((1s,4s)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-ethylphenyl)pyrrolidin-1-yl)-2-azaspiro[3.3]heptan-2-yl)-N-((4-((((1s,4s)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-4-hydroxy-4-methylcyclohexyl)methyl)amino)-3-nitrophenyl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(6-((S)-2-(2-vinylphenyl)pyrrolidin-1-yl)-2-azaspiro[3.3]heptan-2-yl)benzamide,
N—(((R)-3-((2R,5R)-5-methylmorpholin-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N—(((S)-3-((2R,5R)-5-methylmorpholin-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N—(((R)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aS,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-4-(2-((S)-2-phenylpyrrolidin-1-yl)-7-azaspiro[3.5]nonan-7-yl)-2-((5aR,8aS)-5a, 6,8,8a-tetrahydrofuro[3,4-b]pyrrolo [3′, 2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-4-(2-((S)-2-(2-(prop-1-en-2-yl)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-vinylphenyl)pyrrolidin-1-yl)-7-azaspiro[3.5]nonan-7-yl)benzamide,
4-(2-((S)-2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-ethynylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(trifluoromethyl)phenyl)pyrrolidin-1-yl)-7-azaspiro[3.5]nonan-7-yl)benzamide,
4-(2-((S)-2-(2-(tert-butyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(2-methylpent-3-yn-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(1,1,1-trifluoro-2-methylpropan-2-yl)phenyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-4-(2-((S)-2-(2-(prop-1-yn-1-yl)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(2-hydroxypropan-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-phenylpyrrolidin-1-yl)-7-azaspiro[3.5]nonan-7-yl)-2-((5aR,8aS)-5a, 6,8,8a-tetrahydrofuro[3,4-b]pyrrolo [3′, 2′:5,6]pyrido[3,2-e][1,4]oxazin-5(1H)-yl)benzamide,
4-(2-((S)-2-(2-(2-cyanopropan-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(2-aminopropan-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(2-methylbut-3-yn-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-methoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(methylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-(pyridin-2-yl)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(isopropylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-(pyridin-3-yl)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-(pyridin-4-yl)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(3-(trifluoromethyl)bicyclo[1.1.1]pentan-1-yl)phenyl)pyrrolidin-1-yl)-7-azaspiro[3.5]nonan-7-yl)benzamide
4-(2-((S)-2-(2-(bicyclo[1.1.1]pentan-1-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1s,4s)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-isopropoxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-4-isopropoxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-(((4-fluorotetrahydro-2H-pyran-4-yl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((S)-1,4-dioxan-2-yl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((S)-1,4-dioxan-2-yl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((S)-1,4-dioxan-2-yl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1s,4R)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-((1r,4r)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-3-((1r,4R)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-3-((1s,4S)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-3-((1r,4S)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-3-((1s,4R)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-(((3S)-3-((5R)-5-methyl-4-(oxetan-3-yl)morpholin-2-yl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-(((3R)-3-((5R)-5-methyl-4-(oxetan-3-yl)morpholin-2-yl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-3-(morpholinomethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-3-(morpholinomethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((4,4-difluoropiperidin-1-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((4,4-difluoropiperidin-1-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-3-((4-methylpiperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-3-((4-methylpiperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(isopropoxymethyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(isopropoxymethyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-5-nitro-3-(pyridin-2-ylmethyl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-5-nitro-3-(pyridin-2-ylmethyl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((S)-1,4-dioxan-2-yl)methyl)amino)-3-nitrophenyl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-((4-((((1r,4r)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-((4-((((1s,4s)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-5-nitro-3-(tetrahydro-2H-pyran-4-yl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-5-nitro-3-(tetrahydro-2H-pyran-4-yl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((S)-1,4-dioxan-2-yl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((S)-1,4-dioxan-2-yl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-4-hydroxycyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-4-hydroxycyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1s,4R)-4-hydroxycyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxycyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-3-((1r,4R)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-3-((1s,4S)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-3-((1r,4S)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-3-((1s,4R)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-(((3S)-3-((5R)-5-methyl-4-(oxetan-3-yl)morpholin-2-yl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N-(((3R)-3-((5R)-5-methyl-4-(oxetan-3-yl)morpholin-2-yl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-3-(morpholinomethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-3-(morpholinomethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((4,4-difluoropiperidin-1-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((4,4-difluoropiperidin-1-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-3-((4-methylpiperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-3-((4-methylpiperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(isopropoxymethyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(isopropoxymethyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((R)-5-nitro-3-(pyridin-2-ylmethyl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(6-((S)-2-(2-isopropylphenyl)pyrrolidin-1-yl)-2-azaspiro[3.3]heptan-2-yl)-N—(((S)-5-nitro-3-(pyridin-2-ylmethyl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((S)-1,4-dioxan-2-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-(((4-fluorotetrahydro-2H-pyran-4-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((1r,4r)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((1r,4r)-4-isopropoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N—(((S)-5-nitro-3-(tetrahydro-2H-pyran-4-yl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N—(((S)-3-((S)-1,4-dioxan-2-yl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((S)-1,4-dioxan-2-yl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((1s,4R)-4-hydroxycyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N—(((S)-3-((1r,4S)-4-hydroxycyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N—(((R)-3-((1r,4R)-4-hydroxycyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N—(((R)-3-((1s,4S)-4-hydroxycyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((1r,4S)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((1s,4R)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((1s,4S)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((1r,4R)-4-methoxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-(morpholinomethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-(morpholinomethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((4,4-difluoropiperidin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o- tolyl)pyrrolidin-1-yl)-7-azaspiro[3.5]nonan-7-yl)benzamide,
N—(((S)-3-((4,4-difluoropiperidin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o- tolyl)pyrrolidin-1-yl)-7-azaspiro[3.5]nonan-7-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-(isopropoxymethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-(isopropoxymethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-5-nitro-3-(pyridin-2-ylmethyl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-5-nitro-3-(pyridin-2-ylmethyl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((4-isopropylpiperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o- tolyl)pyrrolidin-1-yl)-7-azaspiro[3.5]nonan-7-yl)benzamide,
N—(((S)-3-((4-isopropylpiperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o- tolyl)pyrrolidin-1-yl)-7-azaspiro[3.5]nonan-7-yl)benzamide,
N—(((S)-3-(((1s,4R)-4-hydroxy-4-methylcyclohexyl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-((S)-6-methyl-2-((S)-2-(o-tolyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-((R)-6-methyl-2-((S)-2-(o-tolyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-4,4-dimethyl-2-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2,2-dimethyl-5-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((2S,5S)-2-methyl-5-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-methyl-2-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(4-fluoro-2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(5-fluoro-2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(3-fluoro-2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(4-chloro-2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-3,3-dimethyl-2-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((R)-3,3-dimethyl-2-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((2R)-2-(o-tolyl)-7-oxa-1-azaspiro[4.4]nonan-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((R)-2-(o-tolyl)-8-oxa-1-azaspiro[4.5]decan-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((8R)-8-(o-tolyl)-2-oxa-7-azaspiro[4.4]nonan-7-yl)-7-azaspiro[3.5]nonan-7-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((R)-3-(o-tolyl)-8-oxa-2-azaspiro[4.5]decan-2-yl)-7-azaspiro[3.5]nonan-7-yl)benzamide,
4-(2-((2S)-4-hydroxy-4-methyl-2-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((2S)-4-methoxy-4-methyl-2-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((S)-1,4-dioxan-2-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-4-hydroxy-4-methylcyclohexyl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-(((4-fluorotetrahydro-2H-pyran-4-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((R)-2-fluoro-1,4-dioxan-2-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((S)-2-fluoro-1,4-dioxan-2-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-1-fluoro-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-1-fluoro-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1r,4R)-4-hydroxy-4-methylcyclohexyl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1s,4S)-4-hydroxy-4-methylcyclohexyl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1r,4S)-4-hydroxy-4-methylcyclohexyl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1s,4S)-4-hydroxycyclohexyl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1r,4R)-4-hydroxycyclohexyl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1s,4R)-4-hydroxycyclohexyl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1r,4S)-4-hydroxycyclohexyl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((4-hydroxy-4-methylpiperidin-1-yl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((4-hydroxy-4-methylpiperidin-1-yl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N—(((R)-3-(morpholinomethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N—(((S)-3-(morpholinomethyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydro-5H-furo[3,4-b]furo[3′,2′:5,6]pyrido[3,2-e][1,4]oxazin-5-yl)benzamide,
N—(((R)-3-(isopropoxymethyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(isopropoxymethyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N—(((R)-3-((4-isopropylpiperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N—(((S)-3-((4-isopropylpiperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((4,4-difluoropiperidin-1-yl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((4,4-difluoropiperidin-1-yl)methyl)-5-nitro-3,4-dihydro-2H-benzo[b][1,4]oxazin-7-yl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N—(((R)-3-((4-(isopropylsulfonyl)piperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-414-oxa-7-azaspiro[3.5]nonan-7-yl)-N—(((S)-3-((4-(isopropylsulfonyl)piperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N—(((R)-5-nitro-3-(pyridin-2-ylmethyl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N—(((S)-5-nitro-3-(pyridin-2-ylmethyl)-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
1-(((1R,4s)-4-fluoro-1-methyl-4-((S)-5-nitro-7-(N-(2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzoyl)sulfamoyl)-3,4-dihydro-2H-benzo[b][1,4]oxazin-3-yl)cyclohexyl)oxy)ethyl dihydrogen phosphate,
(1R,4s)-4-fluoro-1-methyl-4-((S)-5-nitro-7-(N-(2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzoyl)sulfamoyl)-3,4-dihydro-2H-benzo[b][1,4]oxazin-3-yl)cyclohexyl dihydrogen phosphate,
((5aR,8aS)-5-(2-(((3-nitro-4-(((tetrahydro-2H-pyran-4-yl)methyl)amino)phenyl)sulfonyl)carbamoyl)-5-(2-((S)-2-(o-tolyl)pyrrolidin-1-yl)-7-azaspiro[3.5]nonan-7-yl)phenyl)-5a,6,8,8a-tetrahydrofuro[3,4-b]pyrrolo[3′,2′:5,6]pyrido[3,2-e][1,4]oxazin-10(5H)-yl)methyl dihydrogen phosphate,
((5aR,8aS)-5-(2-(((3-nitro-4-(((tetrahydro-2H-pyran-4-yl)methyl)amino)phenyl)sulfonyl)carbamoyl)-5-(2-((S)-2-(o-tolyl)pyrrolidin-1-yl)-7-azaspiro[3.5]nonan-7-yl)phenyl)-5a,6,8,8a-tetrahydrofuro[3,4-b]pyrrolo[3′,2′:5,6]pyrido[3,2-e][1,4]oxazin-1(5H)-yl)methyl methyl(3-((phosphonooxy)methyl)pyridin-2-yl)carbamate,
(1R,4s)-4-fluoro-1-methyl-4-((S)-5-nitro-7-(N-(2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzoyl)sulfamoyl)-3,4-dihydro-2H-benzo[b][1,4]oxazin-3-yl)cyclohexyl methyl(3-((phosphonooxy)methyl)pyridin-2-yl)carbamate,
(1R,4s)-4-fluoro-1-methyl-4-((S)-5-nitro-7-(N-(2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzoyl)sulfamoyl)-3,4-dihydro-2H-benzo[b][1,4]oxazin-3-yl)cyclohexyl 2-(4-(phosphonooxy)phenyl)acetate,
((5aR,8aS)-5-(2-(((3-nitro-4-(((tetrahydro-2H-pyran-4-yl)methyl)amino)phenyl)sulfonyl)carbamoyl)-5-(2-((S)-2-(o-tolyl)pyrrolidin-1-yl)-7-azaspiro[3.5]nonan-7-yl)phenyl)-5a,6,8,8a-tetrahydrofuro[3,4-b]pyrrolo[3′,2′:5,6]pyrido[3,2-e][1,4]oxazin-1(5H)-yl)methyl (2-(phosphonooxy)ethyl)(3-((phosphonooxy)methyl)pyridin-2-yl)carbamate,
((5aR,8aS)-5-(2-(((3-nitro-4-(((tetrahydro-2H-pyran-4-yl)methyl)amino)phenyl)sulfonyl)carbamoyl)-5-(2-((S)-2-(o-tolyl)pyrrolidin-1-yl)-7-azaspiro[3.5]nonan-7-yl)phenyl)-5a,6,8,8a-tetrahydrofuro[3,4-b]pyrrolo[3′,2′:5,6]pyrido[3,2-e][1,4]oxazin-1(5H)-yl)methyl 2-(4-(phosphonooxy)phenyl)acetate,
4-(2-((S)-2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-((R)-1,1,1-trifluoro-2-hydroxypropan-2-yl)phenyl)pyrrolidin-1-yl)-7-azaspiro[3.5]nonan-7-yl)benzamide,
4-(2-((S)-2-(2-(1,1,1,3,3,3-hexafluoro-2-hydroxypropan-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-((S)-2,2-dimethyltetrahydro-2H-pyran-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-((R)-2,2-dimethyltetrahydro-2H-pyran-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(isopropoxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-((2,2,2-trifluoroethyl)amino)phenyl)pyrrolidin-1-yl)-7-azaspiro[3.5]nonan-7-yl)benzamide,
4-(2-((S)-2-(2-(methylsulfonamido)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-fluorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(2-methylpent-3-yn-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyanophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(isopropylsulfonyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(dimethylphosphoryl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-carbamoylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(4-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(6-(trifluoromethyl)pyridin-2-yl)phenyl)pyrrolidin-1-yl)-7-azaspiro[3.5]nonan-7-yl)benzamide,
4-(2-((S)-2-(2-(6-methylpyridin-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(5-methylpyridin-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(5-(trifluoromethyl)pyridin-2-yl)phenyl)pyrrolidin-1-yl)-7-azaspiro[3.5]nonan-7-yl)benzamide,
4-(2-((S)-2-(2-(4-methylpyridin-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(4-(trifluoromethyl)pyridin-2-yl)phenyl)pyrrolidin-1-yl)-7-azaspiro[3.5]nonan-7-yl)benzamide,
4-(2-((S)-2-(2-(3-methylpyridin-2-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(3-(trifluoromethyl)pyridin-2-yl)phenyl)pyrrolidin-1-yl)-7-azaspiro[3.5]nonan-7-yl)benzamide,
4-(2-((S)-2-(2-(methylsulfonyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
2-((S)-1-(7-(4-(((3-nitro-4-(((tetrahydro-2H-pyran-4-yl)methyl)amino)phenyl)sulfonyl)carbamoyl)-3-((5aR,8aS)-5a,6,8,8a-tetrahydrofuro[3,4-b]pyrrolo[3′,2′:5,6]pyrido[3,2-e][1,4]oxazin-5(1H)-yl)phenyl)-7-azaspiro[3.5]nonan-2-yl)pyrrolidin-2-yl)benzoic acid,
4-(2-((S)-2-(2-(morpholine-4-carbonyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(4-chloro-2-methylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(3-chloro-2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(4-chloro-2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(3-chloro-2-methylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(bicyclo[1.1.1]pentan-1-yloxy)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(cyclohexyloxy)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isobutoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(trifluoromethoxy)phenyl)pyrrolidin-1-yl)-7-azaspiro[3.5]nonan-7-yl)benzamide,
4-(2-((S)-2-(2-cyclobutoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-(((S)-tetrahydrofuran-3-yl)oxy)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-((tetrahydro-2H-pyran-4-yl)methoxy)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-phenoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-(pyridin-2-yloxy)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-(pyridin-3-yloxy)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-(pyridin-4-yloxy)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(benzyloxy)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-(pyridin-2-ylmethoxy)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((S)-2-(2-(pyridin-2-ylmethyl)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(1-methylcyclopropyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(morpholinomethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
(2S)-1-(7-(4-(((3-nitro-4-(((tetrahydro-2H-pyran-4-yl)methyl)amino)phenyl)sulfonyl)carbamoyl)-3-((5aR,8aS)-5a,6,8,8a-tetrahydrofuro[3,4-b]pyrrolo[3′,2′:5,6]pyrido[3,2-e][1,4]oxazin-5(10H)-yl)phenyl)-7-azaspiro[3.5]nonan-2-yl)-1-((phosphonooxy)methyl)-2-(o-tolyl)pyrrolidin-1-ium,
4-(2-((S)-2-(2-(3-methyl-3-(methylsulfonyl)but-1-yn-1-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(3-morpholinoprop-1-yn-1-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-(bicyclo[1.1.1]pentan-1-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-(3-(trifluoromethyl)bicyclo[1.1.1]pentan-1-yl)phenyl)pyrrolidin-1-yl)-7-azaspiro[3.5]nonan-7-yl)benzamide,
N-((4-((((1r,4r)-1-fluoro-4-methoxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1s,4s)-1-fluoro-4-methoxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((1s,4s)-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((1r,4r)-1-fluoro-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N-((4-((((1s,4s)-1-fluoro-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
N-((4-((((R)-2-fluoro-1,4-dioxan-2-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((S)-2-fluoro-1,4-dioxan-2-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((1r,4R)-1-fluoro-4-hydroxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((1r,4S)-1-fluoro-4-hydroxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-(((1s,4S)-4-hydroxycyclohexyl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-(((1r,4R)-4-hydroxycyclohexyl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-(((1s,4R)-4-hydroxycyclohexyl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-(((1r,4S)-4-hydroxycyclohexyl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-(((1s,4S)-4-hydroxy-4-methylcyclohexyl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-(((1r,4R)-4-hydroxy-4-methylcyclohexyl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-(((1r,4S)-4-hydroxy-4-methylcyclohexyl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-(((1s,4R)-4-hydroxy-4-methylcyclohexyl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((4-(isopropylsulfonyl)piperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((4-(isopropylsulfonyl)piperazin-1-yl)methyl)-5-nitro-3,4-dihydro-2H-benzo[b][1,4]oxazin-7-yl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((R)-3-(o-tolyl)morpholino)-7-azaspiro[3.5]nonan-7-yl)benzamide,
4-(2-((R)-3-(2-isopropylphenyl)morpholino)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((R)-3-(2-isopropoxyphenyl)morpholino)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((R)-3-(o-tolyl)-1,4-oxazepan-4-yl)-7-azaspiro[3.5]nonan-7-yl)benzamide,
4-(2-((R)-3-(2-isopropylphenyl)-1,4-oxazepan-4-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((R)-3-(2-isopropoxyphenyl)-1,4-oxazepan-4-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-3-(2-isopropylphenyl)morpholino)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-3-(2-isopropoxyphenyl)morpholino)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-3-(2-isopropoxyphenyl)morpholino)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((3S,5S)-3-methyl-5-(o-tolyl)morpholino)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((2S,6S)-4-isopropyl-2-methyl-6-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-3,3-dimethyl-5-(o-tolyl)morpholino)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)-4,4-dimethylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)-5,5-dimethylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((2S,5R)-2-(2-isopropylphenyl)-5-methoxy-5-methylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((2S)-2-(2-isopropylphenyl)octahydro-1H-pyrano[3,4-b]pyridin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)azepan-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)-5,5-dimethylazepan-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
2-((5aS,8aR)-8,8-dimethyl-5a,6,8,8a-tetrahydrofuro[3,4-b]pyrrolo[3′,2′:5,6]pyrido[3,2-e][1,4]oxazin-5(1H)-yl)-N-((3-nitro-4-(((tetrahydro-2H-pyran-4-yl)methyl)amino)phenyl)sulfonyl)-4-(2-((R)-3-(o-tolyl)morpholino)-7-azaspiro[3.5]nonan-7-yl)benzamide,
2-((5aR,8aS)-8,8-dimethyl-5a,6,8,8a-tetrahydrofuro[3,4-b]pyrrolo[3′,2′:5,6]pyrido[3,2-e][1,4]oxazin-5(1H)-yl)-N-((3-nitro-4-(((tetrahydro-2H-pyran-4-yl)methyl)amino)phenyl)sulfonyl)-4-(2-((R)-3-(o-tolyl)morpholino)-7-azaspiro[3.5]nonan-7-yl)benzamide
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-3-(o-tolyl)morpholino)-7-azaspiro[3.5]nonan-7-yl)benzamide,
N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-3-(o-tolyl)-1,4-oxazepan-4-yl)-7-azaspiro[3.5]nonan-7-yl)benzamide,
4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(4-isopropylthiophen-3-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylthiophen-3-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-((S)-6-methyl-2-((S)-2-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-((R)-6-methyl-2-((S)-2-(o-tolyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((R)-3-(((R)-3-methyl-4-(oxetan-3-yl)piperazin-1-yl)methyl)-5-nitro-3,4-dihydro-2H-benzo[b][1,4]oxazin-7-yl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N—(((R)-3-(((R)-3-methyl-4-(oxetan-3-yl)piperazin-1-yl)methyl)-5-nitro-3,4-dihydro-2H-benzo[b][1,4]oxazin-7-yl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
4-(2-((S)-2-(2-ethylphenyl)pyrrolidin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((S)-2-(2-(prop-1-yn-1-yl)phenyl)pyrrolidin-1-yl)-7-azaspiro[3.5]nonan-7-yl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-4-(2-((S)-2-(2-(prop-1-en-2-yl)phenyl)pyrrolidin-1-yl)-7-azaspiro[3.5]nonan-7-yl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N—(((S)-3-(((R)-3-methyl-4-(oxetan-3-yl)piperazin-1-yl)methyl)-5-nitro-3,4-dihydro-2H-benzo[b][1,4]oxazin-7-yl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N—(((S)-3-(((R)-3-methyl-4-(oxetan-3-yl)piperazin-1-yl)methyl)-5-nitro-3,4-dihydro-2H-benzo[b][1,4]oxazin-7-yl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(2-vinylphenyl)pyrrolidin-1-yl)-7-azaspiro[3.5]nonan-7-yl)benzamide,
4-(2-((S)-2-(2-(tert-butyl)phenyl)pyrrolidin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
(S)-2-(furo[2,3-b]pyrrolo[3′,2′:5,6]pyrido[3,2-e][1,4]oxazin-4(8H)-yl)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide,
N-((4-((((1r,4r)-4-hydroxy-4-methylcyclohexyl)methyl)amino)-3-nitrophenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-5-nitro-3-(tetrahydro-2H-pyran-4-yl)-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-5-nitro-3-(tetrahydro-2H-pyran-4-yl)-3,4-dihydro-2H-benzo[b][1,4]oxazin-7-yl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin- 1-yl)-7-azaspiro[3.5]nonan-7-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N—(((R)-3-((4-methylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide,
2-((6aS,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide,
2-((6aR,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide,
2-((6aS,10aR)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide,
2-((6aR,10aS)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide,
2-((6aS,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide,
2-((6aR,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-((S)-1,4-dioxan-2-yl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((S)-1,4-dioxan-2-yl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N—(((S)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N—(((R)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
N—(((R)-3-((1r,4R)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
N—(((S)-3-((1s,4R)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N—(((S)-3-((1r,4S)-4-hydroxycyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N—(((R)-3-(morpholinomethyl)-5-nitro-3,4-dihydro-2H-benzo[b][1,4]oxazin-7-yl)sulfonyl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)-N—(((S)-3-(morpholinomethyl)-5-nitro-3,4-dihydro-2H-benzo[b][1,4]oxazin-7-yl)sulfonyl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((6aS,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((6aR,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropylphenyl)pyrrolidin-1-yl)-7-azaspiro[3.5]nonan-7-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropylphenyl)pyrrolidin-1-yl)-7-azaspiro[3.5]nonan-7-yl)-N—(((S)-3-((4-methylpiperazin-1-yl)methyl)-5-nitro-3,4-dihydro-2H-benzo[b][1,4]oxazin-7-yl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(isopropoxymethyl)-5-nitro-3,4-dihydro-2H-benzo[b][1,4]oxazin-7-yl)sulfonyl)-4-(2-((S)-2-(2-isopropylphenyl)pyrrolidin-1-yl)-7-azaspiro[3.5]nonan-7-yl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropylphenyl)pyrrolidin-1-yl)-7-azaspiro[3.5]nonan-7-yl)-N—(((S)-5-nitro-3-(pyridin-2-ylmethyl)-3,4-dihydro-2H-benzo[b][1,4]oxazin-7-yl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-ethylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((S)-1,4-dioxan-2-yl)-5-nitro-3,4-dihydro-2H-benzo[b][1,4]oxazin-7-yl)sulfonyl)-4-(2-((S)-2-(2-ethylphenyl)pyrrolidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((S)-1,4-dioxan-2-yl)-5-nitro-3,4-dihydro-2H-benzo[b][1,4]oxazin-7-yl)sulfonyl)-4-(2-((S)-2-(2-ethylphenyl)pyrrolidin-1-yl)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-ethylphenyl)pyrrolidin-1-yl)-7-azaspiro[3.5]nonan-7-yl)-N—(((S)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-ethylphenyl)pyrrolidin-1-yl)-7-azaspiro[3.5]nonan-7-yl)-N—(((R)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-ethylphenyl)pyrrolidin-1-yl)-7-azaspiro[3.5]nonan-7-yl)-N—(((R)-3-((1s,4S)-4-hydroxycyclohexyl)-5-nitro-3,4-dihydro-2H-benzo[b][1,4]oxazin-7-yl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N—(((R)-3-((1r,4R)-4-hydroxycyclohexyl)-5-nitro-3,4-dihydro-2H-benzo[b][1,4]oxazin-7-yl)sulfonyl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N—(((S)-3-((1s,4R)-4-hydroxycyclohexyl)-5-nitro-3,4-dihydro-2H-benzo[b][1,4]oxazin-7-yl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N—(((S)-3-((1r,4S)-4-hydroxycyclohexyl)-5-nitro-3,4-dihydro-2H-benzo[b][1,4]oxazin-7-yl)sulfonyl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N—(((R)-3-((1r,4R)-4-hydroxy-4-methylcyclohexyl)-5-nitro-3,4-dihydro-2H-benzo[b][1,4]oxazin-7-yl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-ethylphenyl)pyrrolidin-1-yl)-7-azaspiro[3.5]nonan-7-yl)-N—(((R)-3-((1s,4S)-4-hydroxy-4-methylcyclohexyl)-5-nitro-3,4-dihydro-2H-benzo[b][1,4]oxazin-7-yl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-ethylphenyl)pyrrolidin-1-yl)-7-azaspiro[3.5]nonan-7-yl)-N—(((S)-3-((1r,4S)-4-hydroxy-4-methylcyclohexyl)-5-nitro-3,4-dihydro-2H-benzo[b][1,4]oxazin-7-yl)sulfonyl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-ethylphenyl)pyrrolidin-1-yl)-7-azaspiro[3.5]nonan-7-yl)-N—(((S)-3-((1s,4R)-4-hydroxy-4-methylcyclohexyl)-5-nitro-3,4-dihydro-2H-benzo[b][1,4]oxazin-7-yl)sulfonyl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-ethylphenyl)pyrrolidin-1-yl)-7-azaspiro[3.5]nonan-7-yl)-2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N—(((R)-3-(morpholinomethyl)-5-nitro-3,4-dihydro-2H-benzo[b][1,4]oxazin-7-yl)sulfonyl)benzamide,
4-(2-((S)-2-(2-ethylphenyl)pyrrolidin-1-yl)-7-azaspiro[3.5]nonan-7-yl)-2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N—(((S)-3-(morpholinomethyl)-5-nitro-3,4-dihydro-2H-benzo[b][1,4]oxazin-7-yl)sulfonyl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-ethylphenyl)pyrrolidin-1-yl)-7-azaspiro[3.5]nonan-7-yl)-2-((6aS,10aR)-6,6a,7,8,10,10a-hexahydropyrano[4,3-f]pyrrolo[3′,2′:5,6]pyrido[2,3-b][1,4]oxazepin-5(1H)-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-ethylphenyl)pyrrolidin-1-yl)-7-azaspiro[3.5]nonan-7-yl)-2-((6aR,10aS)-6,6a,7,8,10,10a-hexahydropyrano[4,3-f]pyrrolo[3′,2′:5,6]pyrido[2,3-b][1,4]oxazepin-5(1H)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-ethylphenyl)pyrrolidin-1-yl)-7-azaspiro[3.5]nonan-7-yl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-ethylphenyl)pyrrolidin-1-yl)-7-azaspiro[3.5]nonan-7-yl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-ethylphenyl)pyrrolidin-1-yl)-7-azaspiro[3.5]nonan-7-yl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-ethylphenyl)pyrrolidin-1-yl)-7-azaspiro[3.5]nonan-7-yl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-ethylphenyl)pyrrolidin-1-yl)-7-azaspiro[3.5]nonan-7-yl)-N—(((R)-3-(((R)-3-methyl-4-(oxetan-3-yl)piperazin-1-yl)methyl)-5-nitro-3,4-dihydro-2H-benzo[b][1,4]oxazin-7-yl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N—(((S)-3-(((R)-3-methyl-4-(oxetan-3-yl)piperazin-1-yl)methyl)-5-nitro-3,4-dihydro-2H-benzo[b][1,4]oxazin-7-yl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2-(2-ethylphenyl)pyrrolidin-1-yl)-7-azaspiro[3.5]nonan-7-yl)-N—(((R)-3-((4-methylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-ethylphenyl)pyrrolidin-1-yl)-7-azaspiro[3.5]nonan-7-yl)-N—(((S)-3-((4-methylpiperazin-1-yl)methyl)-5-nitro-3,4-dihydro-2H-benzo[b][1,4]oxazin-7-yl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-ethylphenyl)pyrrolidin-1-yl)-7-azaspiro[3.5]nonan-7-yl)-N—(((S)-3-(isopropoxymethyl)-5-nitro-3,4-dihydro-2H-benzo[b][1,4]oxazin-7-yl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-ethylphenyl)pyrrolidin-1-yl)-7-azaspiro[3.5]nonan-7-yl)-N—(((S)-5-nitro-3-(pyridin-2-ylmethyl)-3,4-dihydro-2H-benzo[b][1,4]oxazin-7-yl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-methoxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N—(((S)-3-((S)-1,4-dioxan-2-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N—(((R)-3-((S)-1,4-dioxan-2-yl)-5-nitro-3,4-dihydro-2H-benzo[b][1,4]oxazin-7-yl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N—(((S)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N—(((R)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N—(((R)-3-((1s,4S)-4-hydroxycyclohexyl)-5-nitro-3,4-dihydro-2H-benzo[b][1,4]oxazin-7-yl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((1r,4R)-4-hydroxycyclohexyl)-5-nitro-3,4-dihydro-2H-benzo[b][1,4]oxazin-7-yl)sulfonyl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((1s,4R)-4-hydroxycyclohexyl)-5-nitro-3,4-dihydro-2H-benzo[b][1,4]oxazin-7-yl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((1r,4S)-4-hydroxycyclohexyl)-5-nitro-3,4-dihydro-2H-benzo[b][1,4]oxazin-7-yl)sulfonyl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N—(((R)-3-(morpholinomethyl)-5-nitro-3,4-dihydro-2H-benzo[b][1,4]oxazin-7-yl)sulfonyl)-4-(2-((S)-2-(o-tolyl)pyrrolidin-1-yl)-7-azaspiro[3.5]nonan-7-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N—(((S)-3-(morpholinomethyl)-5-nitro-3,4-dihydro-2H-benzo[b][1,4]oxazin-7-yl)sulfonyl)-4-(2-((S)-2-(o-tolyl)pyrrolidin-1-yl)-7-azaspiro[3.5]nonan-7-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((6aS,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(o-tolyl)pyrrolidin-1-yl)-7-azaspiro[3.5]nonan-7-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((6aR,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(o-tolyl)pyrrolidin-1-yl)-7-azaspiro[3.5]nonan-7-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N—(((R)-3-(((R)-3-methyl-4-(oxetan-3-yl)piperazin-1-yl)methyl)-5-nitro-3,4-dihydro-2H-benzo[b][1,4]oxazin-7-yl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-(((R)-3-methyl-4-(oxetan-3-yl)piperazin-1-yl)methyl)-5-nitro-3,4-dihydro-2H-benzo[b][1,4]oxazin-7-yl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((R)-3-((4-methylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N—(((S)-3-((4-methylpiperazin-1-yl)methyl)-5-nitro-3,4-dihydro-2H-benzo[b][1,4]oxazin-7-yl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N—(((S)-3-(isopropoxymethyl)-5-nitro-3,4-dihydro-2H-benzo[b][1,4]oxazin-7-yl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N—(((S)-5-nitro-3-(pyridin-2-ylmethyl)-3,4-dihydro-2H-benzo[b][1,4]oxazin-7-yl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N-((4-((((1r,4r)-4-methoxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((S)-1,4-dioxan-2-yl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((S)-1,4-dioxan-2-yl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((2R,5R)-5-methyl-4-(oxetan-3-yl)morpholin-2-yl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((2R,5R)-5-methyl-4-(oxetan-3-yl)morpholin-2-yl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-4-hydroxycyclohexyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
N—(((R)-3-((1r,4R)-4-hydroxycyclohexyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
N—(((S)-3-((1s,4R)-4-hydroxycyclohexyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N—(((S)-3-((1r,4S)-4-hydroxycyclohexyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N—(((R)-3-(morpholinomethyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N—(((S)-3-(morpholinomethyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((6aS,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-(prop-1-en-2-yl)phenyl)pyrrolidin-1-yl)-7-azaspiro[3.5]nonan-7-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((6aR,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-(prop-1-en-2-yl)phenyl)pyrrolidin-1-yl)-7-azaspiro[3.5]nonan-7-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((R)-3-(((R)-3-methyl-4-(oxetan-3-yl)piperazin-1-yl)methyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide,
N—(((S)-3-(((R)-3-methyl-4-(oxetan-3-yl)piperazin-1-yl)methyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((4-methylpiperazin-1-yl)methyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((4-methylpiperazin-1-yl)methyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(isopropoxymethyl)-5-nitro-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-5-nitro-3-(pyridin-2-ylmethyl)-3,4-dihydro-2H-benzo[b][1,4]oxazin-7-yl)sulfonyl)-4-(2-((S)-2-(2-(prop-1-en-2-yl)phenyl)pyrrolidin-1-yl)-7-azaspiro[3.5]nonan-7-yl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-cyclopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((S)-1,4-dioxan-2-yl)-5-nitro-3,4-dihydro-2H-benzo[b][1,4]oxazin-7-yl)sulfonyl)-4-(2-((S)-2-(2-cyclopropylphenyl)pyrrolidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((S)-1,4-dioxan-2-yl)-5-nitro-3,4-dihydro-2H-benzo[b][1,4]oxazin-7-yl)sulfonyl)-4-(2-((S)-2-(2-cyclopropylphenyl)pyrrolidin-1-yl)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-cyclopropylphenyl)pyrrolidin-1-yl)-7-azaspiro[3.5]nonan-7-yl)-N—(((S)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-cyclopropylphenyl)pyrrolidin-1-yl)-7-azaspiro[3.5]nonan-7-yl)-N—(((R)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-cyclopropylphenyl)pyrrolidin-1-yl)-7-azaspiro[3.5]nonan-7-yl)-N—(((R)-3-((1s,4S)-4-hydroxycyclohexyl)-5-nitro-3,4-dihydro-2H-benzo[b][1,4]oxazin-7-yl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N—(((R)-3-((1r,4R)-4-hydroxycyclohexyl)-5-nitro-3,4-dihydro-2H-benzo[b][1,4]oxazin-7-yl)sulfonyl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N—(((S)-3-((1s,4R)-4-hydroxycyclohexyl)-5-nitro-3,4-dihydro-2H-benzo[b][1,4]oxazin-7-yl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N—(((S)-3-((1r,4S)-4-hydroxycyclohexyl)-5-nitro-3,4-dihydro-2H-benzo[b][1,4]oxazin-7-yl)sulfonyl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-cyclopropylphenyl)pyrrolidin-1-yl)-7-azaspiro[3.5]nonan-7-yl)-N—(((R)-3-((1r,4R)-4-hydroxy-4-methylcyclohexyl)-5-nitro-3,4-dihydro-2H-benzo[b][1,4]oxazin-7-yl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-cyclopropylphenyl)pyrrolidin-1-yl)-7-azaspiro[3.5]nonan-7-yl)-N—(((R)-3-((1s,4S)-4-hydroxy-4-methylcyclohexyl)-5-nitro-3,4-dihydro-2H-benzo[b][1,4]oxazin-7-yl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-cyclopropylphenyl)pyrrolidin-1-yl)-7-azaspiro[3.5]nonan-7-yl)-N—(((S)-3-((1r,4S)-4-hydroxy-4-methylcyclohexyl)-5-nitro-3,4-dihydro-2H-benzo[b][1,4]oxazin-7-yl)sulfonyl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-cyclopropylphenyl)pyrrolidin-1-yl)-7-azaspiro[3.5]nonan-7-yl)-N—(((S)-3-((1s,4R)-4-hydroxy-4-methylcyclohexyl)-5-nitro-3,4-dihydro-2H-benzo[b][1,4]oxazin-7-yl)sulfonyl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-cyclopropylphenyl)pyrrolidin-1-yl)-7-azaspiro[3.5]nonan-7-yl)-2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N—(((R)-3-(morpholinomethyl)-5-nitro-3,4-dihydro-2H-benzo[b][1,4]oxazin-7-yl)sulfonyl)benzamide,
4-(2-((S)-2-(2-cyclopropylphenyl)pyrrolidin-1-yl)-7-azaspiro[3.5]nonan-7-yl)-2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N—(((S)-3-(morpholinomethyl)-5-nitro-3,4-dihydro-2H-benzo[b][1,4]oxazin-7-yl)sulfonyl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-cyclopropylphenyl)pyrrolidin-1-yl)-7-azaspiro[3.5]nonan-7-yl)-2-((6aS,10aR)-6,6a,7,8,10,10a-hexahydropyrano[4,3-f]pyrrolo[3′,2′:5,6]pyrido[2,3-b][1,4]oxazepin-5(1H)-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-cyclopropylphenyl)pyrrolidin-1-yl)-7-azaspiro[3.5]nonan-7-yl)-2-((6aR,10aS)-6,6a,7,8,10,10a-hexahydropyrano[4,3-f]pyrrolo[3′,2′:5,6]pyrido[2,3-b][1,4]oxazepin-5(1H)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-cyclopropylphenyl)pyrrolidin-1-yl)-7-azaspiro[3.5]nonan-7-yl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-cyclopropylphenyl)pyrrolidin-1-yl)-7-azaspiro[3.5]nonan-7-yl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-cyclopropylphenyl)pyrrolidin-1-yl)-7-azaspiro[3.5]nonan-7-yl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((S)-2-(2-cyclopropylphenyl)pyrrolidin-1-yl)-7-azaspiro[3.5]nonan-7-yl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-cyclopropylphenyl)pyrrolidin-1-yl)-7-azaspiro[3.5]nonan-7-yl)-N—(((R)-3-((4-methylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-cyclopropylphenyl)pyrrolidin-1-yl)-7-azaspiro[3.5]nonan-7-yl)-N—(((S)-3-((4-methylpiperazin-1-yl)methyl)-5-nitro-3,4-dihydro-2H-benzo[b][1,4]oxazin-7-yl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-cyclopropylphenyl)pyrrolidin-1-yl)-7-azaspiro[3.5]nonan-7-yl)-N—(((S)-3-(isopropoxymethyl)-5-nitro-3,4-dihydro-2H-benzo[b][1,4]oxazin-7-yl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-cyclopropylphenyl)pyrrolidin-1-yl)-7-azaspiro[3.5]nonan-7-yl)-N—(((S)-5-nitro-3-(pyridin-2-ylmethyl)-3,4-dihydro-2H-benzo[b][1,4]oxazin-7-yl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-ethynylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((S)-1,4-dioxan-2-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(2-(trifluoromethyl)phenyl)pyrrolidin-1-yl)-7-azaspiro[3.5]nonan-7-yl)benzamide,
N—(((R)-3-((S)-1,4-dioxan-2-yl)-5-nitro-3,4-dihydro-2H-benzo[b][1,4]oxazin-7-yl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(2-(1,1,1-trifluoro-2-methylpropan-2-yl)phenyl)pyrrolidin-1-yl)-7-azaspiro[3.5]nonan-7-yl)benzamide,
N—(((S)-3-((2R,5R)-5-methyl-4-(oxetan-3-yl)morpholin-2-yl)-5-nitro-3,4-dihydro-2H-benzo[b][1,4]oxazin-7-yl)sulfonyl)-4-(2-((S)-2-(2-(2-methylpent-3-yn-2-yl)phenyl)pyrrolidin-1-yl)-7-azaspiro[3.5]nonan-7-yl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-(2-hydroxypropan-2-yl)phenyl)pyrrolidin-1-yl)-7-azaspiro[3.5]nonan-7-yl)-N—(((R)-3-((2R,5R)-5-methyl-4-(oxetan-3-yl)morpholin-2-yl)-5-nitro-3,4-dihydro-2H-benzo[b][1,4]oxazin-7-yl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-(2-cyanopropan-2-yl)phenyl)pyrrolidin-1-yl)-7-azaspiro[3.5]nonan-7-yl)-N—(((R)-3-((1s,4S)-4-hydroxycyclohexyl)-5-nitro-3,4-dihydro-2H-benzo[b][1,4]oxazin-7-yl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-(2-cyanopropan-2-yl)phenyl)pyrrolidin-1-yl)-7-azaspiro[3.5]nonan-7-yl)-N—(((R)-3-((1r,4R)-4-hydroxycyclohexyl)-5-nitro-3,4-dihydro-2H-benzo[b][1,4]oxazin-7-yl)sulfonyl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-(2-cyanopropan-2-yl)phenyl)pyrrolidin-1-yl)-7-azaspiro[3.5]nonan-7-yl)-N—(((S)-3-((1s,4R)-4-hydroxycyclohexyl)-5-nitro-3,4-dihydro-2H-benzo[b][1,4]oxazin-7-yl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-(2-cyanopropan-2-yl)phenyl)pyrrolidin-1-yl)-7-azaspiro[3.5]nonan-7-yl)-N—(((S)-3-((1r,4S)-4-hydroxycyclohexyl)-5-nitro-3,4-dihydro-2H-benzo[b][1,4]oxazin-7-yl)sulfonyl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(6-((S)-2-(2-isopropylphenyl)pyrrolidin-1-yl)-2-azaspiro[3.3]heptan-2-yl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)benzamide,
4-(6-((S)-2-(2-cyclopropylphenyl)pyrrolidin-1-yl)-2-azaspiro[3.3]heptan-2-yl)-2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N—(((R)-3-(morpholinomethyl)-5-nitro-3,4-dihydro-2H-benzo[b][1,4]oxazin-7-yl)sulfonyl)benzamide,
N-((3-nitro-4-(((tetrahydro-2H-pyran-4-yl)methyl)amino)phenyl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)-4-(6-((S)-2-(o-tolyl)pyrrolidin-1-yl)-2-azaspiro[3.3]heptan-2-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-4-((S)-6-methyl-2-((S)-2-(o-tolyl)pyrrolidin-1-yl)-7-azaspiro[3.5]nonan-7-yl)-N—(((S)-3-(morpholinomethyl)-5-nitro-3,4-dihydro-2H-benzo[b][1,4]oxazin-7-yl)sulfonyl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
(S)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-(pyrido[3,4-b]pyrrolo[3′,2′:5,6]pyrido[3,2-e][1,4]oxazin-5(1H)-yl)benzamide,
(S)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-(pyrido[4,3-b]pyrrolo[3′,2′:5,6]pyrido[3,2-e][1,4]oxazin-5(1H)-yl)benzamide,
(S)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-(10-oxo-1,10-dihydro-5H-pyrido[3,4-b]pyrrolo[2,3-g][1,5]naphthyridin-5-yl)benzamide,
(S)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-(10-oxo-1,10-dihydro-5H-pyrido[4,3-b]pyrrolo[2,3-g][1,5]naphthyridin-5-yl)benzamide,
N-((3-nitro-4-(((tetrahydro-2H-pyran-4-yl)methyl)amino)phenyl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide,
2-((6aS,10aR)-6,6a,7,8,10,10a-hexahydropyrano[4,3-f]pyrrolo[3′,2′:5,6]pyrido[2,3-b][1,4]oxazepin-5(1H)-yl)-N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)benzamide,
2-((6aR,10aS)-6,6a,7,8,10,10a-hexahydropyrano[4,3-f]pyrrolo[3′,2′:5,6]pyrido[2,3-b][1,4]oxazepin-5(1H)-yl)-N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide
2-((6aS,10aR)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide,
2-((6aR,10aS)-6,6a,7,8,10,10a-hexahydropyrano[4,3-f]pyrrolo[3′,2′:5,6]pyrido[2,3-b][1,4]oxazepin-5(1H)-yl)-4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aR,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aS,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aR,9aR)-6a,7,9,9a-tetrahydro-1H-furo[3,4-f]pyrrolo[3′,2′:5,6]pyrido[2,3-b][1,4]oxazepin-5(6H)-yl)benzamide,
4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((6aS,9aS)-6a,7,9,9a-tetrahydro-1H-furo[3,4-f]pyrrolo[3′,2′:5,6]pyrido[2,3-b][1,4]oxazepin-5(6H)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((R)-6-methyl-2-((R)-3-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((S)-6-methyl-2-((R)-3-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((2S,4S,6R)-6-methyl-2-((R)-3-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((2R,4R,6R)-6-methyl-2-((R)-3-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((2R,4S,6S)-6-methyl-2-((R)-3-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((2S,4R,6S)-6-methyl-2-((R)-3-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(6,6-dimethyl-2-((R)-3-(o-tolyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1R,2S)-2-((R)-3-(2-isopropylphenyl)morpholino)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1R,2R)-2-((R)-3-(2-isopropylphenyl)morpholino)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1S,2S)-2-((R)-3-(2-isopropylphenyl)morpholino)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1S,2R)-2-((R)-3-(2-isopropylphenyl)morpholino)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((S)-2-((R)-3-(2-isopropylphenyl)morpholino)-1,1-dimethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((R)-2-((R)-3-(2-isopropylphenyl)morpholino)-1,1-dimethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-1,1,3,3-tetramethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(9-((R)-3-(2-isopropylphenyl)morpholino)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((R)-8-((R)-3-(2-isopropylphenyl)morpholino)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((S)-8-((R)-3-(2-isopropylphenyl)morpholino)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((S)-2-((R)-3-(2-isopropylphenyl)morpholino)-8-azaspiro[4.5]decan-8-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((R)-2-((R)-3-(2-isopropylphenyl)morpholino)-8-azaspiro[4.5]decan-8-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((S)-1-((R)-3-(2-isopropylphenyl)morpholino)-6-azaspiro[2.5]octan-6-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((3R,5R)-3-(2-isopropylphenyl)-5-methylmorpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((3R,5S)-3-(2-isopropylphenyl)-5-methylmorpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-5-(2-isopropylphenyl)-3,3-dimethylmorpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-5-(2-isopropylphenyl)-2,2-dimethylmorpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1R,4R,6R)-6-(2-isopropylphenyl)-2-oxa-5-azabicyclo[2.2.1]heptan-5-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1S,4S,6R)-6-(2-isopropylphenyl)-2-oxa-5-azabicyclo[2.2.1]heptan-5-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)-4-methylpiperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-isopropyl-2-(2-isopropylphenyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)-4-(isopropylsulfonyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)-1,1-dioxidothiomorpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-hydroxy-2-(2-isopropylphenyl)-4-methylpiperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-hydroxy-2-(2-isopropylphenyl)-4-methylpiperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4R)-2-(2-isopropylphenyl)-4-methoxy-4-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4S)-2-(2-isopropylphenyl)-4-methoxy-4-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)-4,4-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-6-(2-isopropylphenyl)-2,2-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)-5,5-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4,4-difluoro-2-(2-isopropylphenyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-5,5-difluoro-2-(2-isopropylphenyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-ethynylphenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-(1,1,1-trifluoro-2-methylpropan-2-yl)phenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-3-(2-(2-hydroxypropan-2-yl)phenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-(2-aminopropan-2-yl)phenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-(tert-butoxy)phenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-(trifluoromethoxy)phenyl)morpholino)-7-azaspiro[3.5]nonan-7-yl)benzamide,
4-(2-((R)-3-(2-cyclopropoxyphenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-cyclobutoxyphenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-3-(3-isopropylthiophen-2-yl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropylthiophen-3-yl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(4-isopropylthiophen-3-yl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N-((4-(((4-fluorotetrahydro-2H-pyran-4-yl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N-((4-((((S)-1,4-dioxan-2-yl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-((1r,4R)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-((1r,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
(1S,4r)-1-methyl-4-((S)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1R,4r)-1-methyl-4-((R)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1R,4s)-1-methyl-4-((S)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1S,4s)-1-methyl-4-((R)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1S,4s)-4-fluoro-1-methyl-4-((R)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1R,4s)-4-fluoro-1-methyl-4-((S)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1R,4r)-4-fluoro-1-methyl-4-((R)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1S,4r)-4-fluoro-1-methyl-4-((S)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzoyl)sulfamoyl)-3,4-dihydro-2H-benzo[b][1,4]oxazin-3-yl)cyclohexyl dihydrogen phosphate,
N—(((S)-3-(4-fluorotetrahydro-2H-pyran-4-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-(4-fluorotetrahydro-2H-pyran-4-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-(((1r,4R)-4-hydroxy-4-methylcyclohexyl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-(((1s,4S)-4-hydroxy-4-methylcyclohexyl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-(((1r,4S)-4-hydroxy-4-methylcyclohexyl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-(((1s,4S)-4-hydroxycyclohexyl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-(((1s,4R)-4-hydroxy-4-methylcyclohexyl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-(((1r,4R)-4-hydroxycyclohexyl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-(((1s,4R)-4-hydroxycyclohexyl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-(((1r,4S)-4-hydroxycyclohexyl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-((4-hydroxy-4-methylpiperidin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-((4-hydroxy-4-methylpiperidin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-(morpholinomethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-(morpholinomethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-(isopropoxymethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-(isopropoxymethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-((4-isopropylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-((4-isopropylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-((4,4-difluoropiperidin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-((4,4-difluoropiperidin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-3-((4-(isopropylsulfonyl)piperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-3-((4-(isopropylsulfonyl)piperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((R)-5-nitro-3-(pyridin-2-ylmethyl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
N—(((S)-5-nitro-3-(pyridin-2-ylmethyl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide,
4-(2-((R)-3-(2-isopropylphenyl)morpholino)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((S)-1,4-dioxan-2-yl)methyl)amino)-3-nitrophenyl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1S,4r)-4-((S)-7-(N-(4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-((R)-7-(N-(4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-((S)-7-(N-(4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-((R)-7-(N-(4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-fluoro-4-((R)-7-(N-(4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-fluoro-4-((S)-7-(N-(4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-fluoro-4-((R)-7-(N-(4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4r)-4-fluoro-4-((S)-7-(N-(4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
N—(((S)-3-(4-fluorotetrahydro-2H-pyran-4-yl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(4-fluorotetrahydro-2H-pyran-4-yl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1r,4R)-4-hydroxy-4-methylcyclohexyl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1s,4S)-4-hydroxy-4-methylcyclohexyl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1r,4S)-4-hydroxy-4-methylcyclohexyl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1s,4S)-4-hydroxycyclohexyl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1s,4R)-4-hydroxy-4-methylcyclohexyl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1r,4R)-4-hydroxycyclohexyl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1s,4R)-4-hydroxycyclohexyl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1r,4S)-4-hydroxycyclohexyl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((4-hydroxy-4-methylpiperidin-1-yl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((4-hydroxy-4-methylpiperidin-1-yl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropylphenyl)morpholino)-7-azaspiro[3.5]nonan-7-yl)-N—(((R)-3-(morpholinomethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropylphenyl)morpholino)-7-azaspiro[3.5]nonan-7-yl)-N—(((S)-3-(morpholinomethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(isopropoxymethyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(isopropoxymethyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropylphenyl)morpholino)-7-azaspiro[3.5]nonan-7-yl)-N—(((R)-3-((4-isopropylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropylphenyl)morpholino)-7-azaspiro[3.5]nonan-7-yl)-N—(((S)-3-((4-isopropylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((4,4-difluoropiperidin-1-yl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((4,4-difluoropiperidin-1-yl)methyl)-5-nitro-3,4-dihydro-2H-benzo[b][1,4]oxazin-7-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropylphenyl)morpholino)-7-azaspiro[3.5]nonan-7-yl)-N—(((R)-3-((4-(isopropylsulfonyl)piperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropylphenyl)morpholino)-7-azaspiro[3.5]nonan-7-yl)-N—(((S)-3-((4-(isopropylsulfonyl)piperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropylphenyl)morpholino)-7-azaspiro[3.5]nonan-7-yl)-N—(((R)-5-nitro-3-(pyridin-2-ylmethyl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropylphenyl)morpholino)-7-azaspiro[3.5]nonan-7-yl)-N—(((S)-5-nitro-3-(pyridin-2-ylmethyl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropoxyphenyl)morpholino)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((S)-1,4-dioxan-2-yl)methyl)amino)-3-nitrophenyl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1S,4r)-4-((S)-7-(N-(4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-((R)-7-(N-(4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-((S)-7-(N-(4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-((R)-7-(N-(4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-fluoro-4-((R)-7-(N-(4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-fluoro-4-((S)-7-(N-(4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-fluoro-4-((R)-7-(N-(4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4r)-4-fluoro-4-((S)-7-(N-(4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
N—(((S)-3-(4-fluorotetrahydro-2H-pyran-4-yl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(4-fluorotetrahydro-2H-pyran-4-yl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1r,4R)-4-hydroxy-4-methylcyclohexyl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1s,4S)-4-hydroxy-4-methylcyclohexyl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1r,4S)-4-hydroxy-4-methylcyclohexyl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1s,4S)-4-hydroxycyclohexyl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1s,4R)-4-hydroxy-4-methylcyclohexyl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1r,4R)-4-hydroxycyclohexyl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1s,4R)-4-hydroxycyclohexyl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1r,4S)-4-hydroxycyclohexyl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((4-hydroxy-4-methylpiperidin-1-yl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((4-hydroxy-4-methylpiperidin-1-yl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropoxyphenyl)morpholino)-7-azaspiro[3.5]nonan-7-yl)-N—(((R)-3-(morpholinomethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropoxyphenyl)morpholino)-7-azaspiro[3.5]nonan-7-yl)-N—(((S)-3-(morpholinomethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(isopropoxymethyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(isopropoxymethyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropoxyphenyl)morpholino)-7-azaspiro[3.5]nonan-7-yl)-N—(((R)-3-((4-isopropylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropoxyphenyl)morpholino)-7-azaspiro[3.5]nonan-7-yl)-N—(((S)-3-((4-isopropylpiperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((4,4-difluoropiperidin-1-yl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((4,4-difluoropiperidin-1-yl)methyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropoxyphenyl)morpholino)-7-azaspiro[3.5]nonan-7-yl)-N—(((R)-3-((4-(isopropylsulfonyl)piperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropoxyphenyl)morpholino)-7-azaspiro[3.5]nonan-7-yl)-N—(((S)-3-((4-(isopropylsulfonyl)piperazin-1-yl)methyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropoxyphenyl)morpholino)-7-azaspiro[3.5]nonan-7-yl)-N—(((R)-5-nitro-3-(pyridin-2-ylmethyl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-isopropoxyphenyl)morpholino)-7-azaspiro[3.5]nonan-7-yl)-N—(((S)-5-nitro-3-(pyridin-2-ylmethyl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N-((4-(((4-fluorotetrahydro-2H-pyran-4-yl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N-((4-((((S)-1,4-dioxan-2-yl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((R)-3-(4-fluorotetrahydro-2H-pyran-4-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
(1S,4r)-1-methyl-4-((S)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1R,4r)-1-methyl-4-((R)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1S,4s)-4-fluoro-1-methyl-4-((R)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzoyl)sulfamoyl)-3,4-dihydro-2H-benzo[b][1,4]oxazin-3-yl)cyclohexyl dihydrogen phosphate,
(1R,4s)-4-fluoro-1-methyl-4-((S)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzoyl)sulfamoyl)-3,4-dihydro-2H-benzo[b][1,4]oxazin-3-yl)cyclohexyl dihydrogen phosphate,
4-(2-((R)-3-(2-isopropylphenyl)-1,4-oxazepan-4-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((S)-1,4-dioxan-2-yl)methyl)amino)-3-nitrophenyl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(4-fluorotetrahydro-2H-pyran-4-yl)-5-nitro-3,4-dihydro-2H-benzo[b][1,4]oxazin-7-yl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1S,4r)-4-((S)-7-(N-(4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-((R)-7-(N-(4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-fluoro-4-((R)-7-(N-(4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-fluoro-4-((S)-7-(N-(4-(2-((R)-3-(2-isopropylphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
4-(2-((R)-3-(2-isopropoxyphenyl)-1,4-oxazepan-4-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((S)-1,4-dioxan-2-yl)methyl)amino)-3-nitrophenyl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(4-fluorotetrahydro-2H-pyran-4-yl)-5-nitro-3,4-dihydro-2H-benzo[b][1,4]oxazin-7-yl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1S,4r)-4-((S)-7-(N-(4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-((R)-7-(N-(4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-fluoro-4-((R)-7-(N-(4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-fluoro-4-((S)-7-(N-(4-(2-((R)-3-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
4-((2R,4S,6S)-6-methyl-2-((R)-3-(o-tolyl)morpholino)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-((2S,4R,6S)-6-methyl-2-((R)-3-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((S)-1,4-dioxan-2-yl)methyl)amino)-3-nitrophenyl)sulfonyl)-4-((2S,4S,6R)-6-methyl-2-((R)-3-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-((2R,4R,6R)-6-methyl-2-((R)-3-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((S)-1,1-dimethyl-2-((R)-3-(o-tolyl)morpholino)-7-azaspiro[3.5]nonan-7-yl)-N—(((R)-3-((1r,4R)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((R)-1,1-dimethyl-2-((R)-3-(o-tolyl)morpholino)-7-azaspiro[3.5]nonan-7-yl)-N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3,3-dimethyl-5-(o-tolyl)morpholino)-7-azaspiro[3.5]nonan-7-yl)-N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1S,4r)-4-((S)-7-(N-(4-(2-((R)-2,2-dimethyl-5-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-1-methyl-4-((R)-7-(N-(4-(2-((3R,5R)-3-methyl-5-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)cyclohexyl dihydrogen phosphate,
(1S,4s)-4-fluoro-1-methyl-4-((R)-7-(N-(4-(2-((3S,5R)-3-methyl-5-(o-tolyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)cyclohexyl dihydrogen phosphate,
(1R,4s)-4-fluoro-1-methyl-4-((S)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(o-tolyl)piperidin-1-yl)-7-azaspiro[3.5]nonan-7-yl)benzoyl)sulfamoyl)-3,4-dihydro-2H-benzo[b][1,4]oxazin-3-yl)cyclohexyl dihydrogen phosphate,
4-(2-((S)-4,4-dimethyl-2-(o-tolyl)piperidin-1-yl)-7-azaspiro[3.5]nonan-7-yl)-N—(((S)-3-(4-fluorotetrahydro-2H-pyran-4-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-5,5-dimethyl-2-(o-tolyl)piperidin-1-yl)-7-azaspiro[3.5]nonan-7-yl)-N—(((S)-3-(4-fluorotetrahydro-2H-pyran-4-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-6-(2-isopropylphenyl)-2,2-dimethylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-(2-((2S,6S)-2-(2-isopropylphenyl)-6-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((S)-1,4-dioxan-2-yl)methyl)amino)-3-nitrophenyl)sulfonyl)-4-(2-((2S,6R)-2-(2-isopropylphenyl)-6-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-hydroxy-2-(2-isopropylphenyl)-4-methylpiperidin-1-yl)-7-azaspiro[3.5]nonan-7-yl)-N—(((S)-3-((1r,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-hydroxy-2-(2-isopropylphenyl)-4-methylpiperidin-1-yl)-7-azaspiro[3.5]nonan-7-yl)-N—(((R)-3-((1r,4R)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-(2-((2S,5S)-5-hydroxy-2-(2-isopropylphenyl)-5-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-(2-((2S,5R)-5-hydroxy-2-(2-isopropylphenyl)-5-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1S,4r)-4-((S)-7-(N-(4-(2-((2S,5S)-2-(2-isopropylphenyl)-5-methoxy-5-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-((R)-7-(N-(4-(2-((2S,5R)-2-(2-isopropylphenyl)-5-methoxy-5-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-fluoro-4-((R)-7-(N-(4-(2-((2S,4R)-2-(2-isopropylphenyl)-4-methoxy-4-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-fluoro-4-((S)-7-(N-(4-(2-((2S,4S)-2-(2-isopropylphenyl)-4-methoxy-4-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
4-(2-((S)-4,4-difluoro-2-(2-isopropylphenyl)piperidin-1-yl)-7-azaspiro[3.5]nonan-7-yl)-N—(((S)-3-(4-fluorotetrahydro-2H-pyran-4-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-5,5-difluoro-2-(2-isopropylphenyl)piperidin-1-yl)-7-azaspiro[3.5]nonan-7-yl)-N—(((S)-3-(4-fluorotetrahydro-2H-pyran-4-yl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-methylpiperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-(2-((R)-2-(2-isopropoxyphenyl)-4-isopropylpiperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((S)-1,4-dioxan-2-yl)methyl)amino)-3-nitrophenyl)sulfonyl)-4-(2-((R)-2-(2-isopropoxyphenyl)-4-(isopropylsulfonyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)-1,1-dioxidothiomorpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-1,1,3,3-tetramethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-((1R,2S)-2-((R)-3-(2-isopropoxyphenyl)morpholino)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-((1R,2R)-2-((R)-3-(2-isopropoxyphenyl)morpholino)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1S,4r)-4-((S)-7-(N-(4-((1S,2R)-2-((R)-3-(2-isopropoxyphenyl)morpholino)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-((R)-7-(N-(4-((1S,2S)-2-((R)-3-(2-isopropoxyphenyl)morpholino)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-fluoro-4-((R)-7-(N-(4-(9-((R)-3-(2-isopropoxyphenyl)morpholino)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-fluoro-4-((S)-7-(N-(4-((S)-8-((R)-3-(2-isopropoxyphenyl)morpholino)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
N—(((S)-3-(4-fluorotetrahydro-2H-pyran-4-yl)-5-nitro-3,4-dihydro-2H-benzo[b][1,4]oxazin-7-yl)sulfonyl)-4-((R)-8-((R)-3-(2-isopropoxyphenyl)morpholino)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-((2R)-2-((R)-3-(2-isopropoxyphenyl)morpholino)-7-azaspiro[4.5]decan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((2S)-2-((R)-3-(2-isopropoxyphenyl)morpholino)-7-azaspiro[4.5]decan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-((R)-1-((R)-3-(o-tolyl)-1,4-oxazepan-4-yl)-6-azaspiro[2.5]octan-6-yl)benzamide,
N-((4-((((S)-1,4-dioxan-2-yl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-((S)-1-((R)-3-(o-tolyl)-1,4-oxazepan-4-yl)-6-azaspiro[2.5]octan-6-yl)benzamide,
N-((4-((((1r,4r)-4-hydroxy-4-methylcyclohexyl)methyl)amino)-3-nitrophenyl)sulfonyl)-4-(2-((1R,4R,6R)-6-(2-isopropylphenyl)-2-oxa-5-azabicyclo[2.2.1]heptan-5-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-(2-((1S,4S,6R)-6-(2-isopropylphenyl)-2-oxa-5-azabicyclo[2.2.1]heptan-5-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-(2-((S)-3-(3-isopropylthiophen-2-yl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-3-(4-isopropylthiophen-3-yl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((R)-3-(2-isopropylthiophen-3-yl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-ethynylphenyl)-1,4-oxazepan-4-yl)-7-azaspiro[3.5]nonan-7-yl)-N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-(1,1,1-trifluoro-2-methylpropan-2-yl)phenyl)-1,4-oxazepan-4-yl)-7-azaspiro[3.5]nonan-7-yl)benzamide,
(1S,4r)-4-((S)-7-(N-(4-(2-((R)-3-(2-(2-hydroxypropan-2-yl)phenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-((R)-7-(N-(4-(2-((R)-3-(2-(2-aminopropan-2-yl)phenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-((R)-7-(N-(4-(2-((R)-3-(2-(tert-butoxy)phenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-4-fluoro-1-methylcyclohexyl dihydrogen phosphate,
(1R,4s)-4-fluoro-1-methyl-4-((S)-5-nitro-7-(N-(2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-(trifluoromethoxy)phenyl)-1,4-oxazepan-4-yl)-7-azaspiro[3.5]nonan-7-yl)benzoyl)sulfamoyl)-3,4-dihydro-2H-benzo[b][1,4]oxazin-3-yl)cyclohexyl dihydrogen phosphate,
4-(2-((R)-3-(2-cyclopropoxyphenyl)-1,4-oxazepan-4-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-cyclobutoxyphenyl)-1,4-oxazepan-4-yl)-7-azaspiro[3.5]nonan-7-yl)-N-((4-(((4-fluorotetrahydro-2H-pyran-4-yl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((S)-1,4-dioxan-2-yl)methyl)amino)-3-nitrophenyl)sulfonyl)-4-((2S,4S,6R)-2-((R)-3-(2-isopropylphenyl)-1,4-oxazepan-4-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((2R,4R,6R)-2-((R)-3-(2-isopropylphenyl)-1,4-oxazepan-4-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-((2R,4S,6S)-2-((R)-3-(2-isopropylphenyl)-1,4-oxazepan-4-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-((2S,4R,6S)-2-((R)-3-(2-isopropylphenyl)-1,4-oxazepan-4-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-((S)-2-((R)-3-(2-isopropylphenyl)-1,4-oxazepan-4-yl)-1,1-dimethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-((R)-2-((R)-3-(2-isopropylphenyl)-1,4-oxazepan-4-yl)-1,1-dimethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropylphenyl)-1,4-oxazepan-4-yl)-1,1,3,3-tetramethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-((1R,2R)-2-((R)-3-(2-isopropylphenyl)-1,4-oxazepan-4-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1S,4r)-4-((S)-7-(N-(4-((1S,2R)-2-((R)-3-(2-isopropylphenyl)-1,4-oxazepan-4-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-((R)-7-(N-(4-((1R,2S)-2-((R)-3-(2-isopropylphenyl)-1,4-oxazepan-4-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-fluoro-4-((R)-7-(N-(4-((1S,2S)-2-((R)-3-(2-isopropylphenyl)-1,4-oxazepan-4-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-fluoro-4-((S)-7-(N-(4-(2-((R)-3-(2-isopropylphenyl)-5,5-dimethyl-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
4-(2-((R)-3-(2-isopropoxyphenyl)-6,6-dimethyl-1,4-oxazepan-4-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-(2-((R)-3-(2-isopropoxyphenyl)-7,7-dimethyl-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((S)-1,4-dioxan-2-yl)methyl)amino)-3-nitrophenyl)sulfonyl)-4-(2-((S)-5-(2-isopropoxyphenyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5S)-5-hydroxy-2-(2-isopropoxyphenyl)-5-methylazepan-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-(2-((2S,5R)-5-hydroxy-2-(2-isopropoxyphenyl)-5-methylazepan-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-(4-fluorotetrahydro-2H-pyran-4-yl)-5-nitro-3,4-dihydro-2H-benzo[b][1,4]oxazin-7-yl)sulfonyl)-4-(2-((S)-2-(2-isopropoxyphenyl)-5,5-dimethylazepan-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-((2S,4S,6R)-2-((R)-3-(2-isopropoxyphenyl)-5,5-dimethyl-1,4-oxazepan-4-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-((2R,4S,6S)-2-((R)-3-(2-isopropoxyphenyl)-5,5-dimethyl-1,4-oxazepan-4-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-((2R,4R,6R)-2-((R)-3-(2-isopropoxyphenyl)-5,5-dimethyl-1,4-oxazepan-4-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-((2S,4R,6S)-2-((R)-3-(2-isopropoxyphenyl)-5,5-dimethyl-1,4-oxazepan-4-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1S,4r)-4-((S)-7-(N-(4-(9-((3R,5R)-3-(2-isopropoxyphenyl)-5-methyl-1,4-oxazepan-4-yl)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4r)-4-((R)-7-(N-(4-(9-((3R,5S)-3-(2-isopropoxyphenyl)-5-methyl-1,4-oxazepan-4-yl)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1S,4s)-4-fluoro-4-((R)-7-(N-(4-((1R,2R)-2-((R)-3-(2-isopropoxyphenyl)-5,5-dimethyl-1,4-oxazepan-4-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
(1R,4s)-4-fluoro-4-((S)-7-(N-(4-((1S,2S)-2-((R)-3-(2-isopropoxyphenyl)-5,5-dimethyl-1,4-oxazepan-4-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzoyl)sulfamoyl)-5-nitro-3,4-dihydro-2H-benzo[b][1,4]oxazin-3-yl)-1-methylcyclohexyl dihydrogen phosphate,
N-((4-((((1r,4r)-4-hydroxy-4-methylcyclohexyl)methyl)amino)-3-nitrophenyl)sulfonyl)-4-(7-((R)-3-(2-isopropylphenyl)morpholino)-2-azaspiro[3.5]nonan-2-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(6-((R)-3-(2-isopropylphenyl)morpholino)-2-azaspiro[3.3]heptan-2-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)-1,4-oxazepan-4-yl)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-3-(2-isopropoxyphenyl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-3-(2-isopropoxyphenyl)-1,4-oxazepan-4-yl)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)-4-(2-((R)-3-(o-tolyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)-4-(2-((R)-3-(o-tolyl)-1,4-oxazepan-4-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-3-(3-isopropylthiophen-2-yl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-3-(2-isopropylthiophen-3-yl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-3-(4-isopropylthiophen-3-yl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)-4-(2-((S)-3-(o-tolyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-3-(2-isopropoxyphenyl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-3-(2-isopropylphenyl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-3-(2-isopropylphenyl)morpholino)-1,1,3,3-tetramethyl-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-((1R,2S)-2-((S)-3-(2-isopropylphenyl)morpholino)-1-methyl-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-((1R,2R)-2-((S)-3-(2-isopropylphenyl)morpholino)-1-methyl-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-((1S,2S)-2-((S)-3-(2-isopropylphenyl)morpholino)-1-methyl-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((3S,5S)-3-(2-isopropylphenyl)-5-methylmorpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1 r,4r)-4-hydroxy-4-methylcyclohexyl)methyl)amino)-3-nitrophenyl)sulfonyl)-4-((1 S,2R)-2-((S)-3-(2-isopropylphenyl)morpholino)-1-methyl-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((3S,5R)-3-(2-isopropylphenyl)-5-methylmorpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((S)-1,2,4a,5-tetrahydro-4H-pyrrolo[3″,2″:5′,6′]pyrido[3′,2′:5,6]pyrazino[2,1-c][1,4]oxazin-6(10H)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((R)-1,2,4a,5-tetrahydro-4H-pyrrolo[3″,2″:5′,6′]pyrido[3′,2′:5,6]pyrazino[2,1-c][1,4]oxazin-6(10H)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((S)-2,3,5a,6-tetrahydro-1H,5H-pyrrolo[3″,2″:5′,6′]pyrido[3′,2′:5,6]pyrazino[2,1-c][1,4]oxazepin-7(11H)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((R)-2,3,5a,6-tetrahydro-1H,5H-pyrrolo[3″,2″:5′,6′]pyrido[3′,2′:5,6]pyrazino[2,1-c][1,4]oxazepin-7(11H)-yl)benzamide,
2-((5aR,10aR)-5a,6,7,9,10,10a-hexahydrooxepino[4,5-b]pyrrolo[3′,2′:5,6]pyrido[3,2-e][1,4]oxazin-5(1H)-yl)-4-(2-((R)-3-(2-isopropylphenyl)morpholino)-7-azaspiro[3.5]nonan-7-yl)-N-((3-nitro-4-(((tetrahydro-2H-pyran-4-yl)methyl)amino)phenyl)sulfonyl)benzamide,
2-((5aS,10aS)-5a,6,7,9,10,10a-hexahydrooxepino[4,5-b]pyrrolo[3′,2′:5,6]pyrido[3,2-e][1,4]oxazin-5(1H)-yl)-4-(2-((R)-3-(2-isopropylphenyl)morpholino)-7-azaspiro[3.5]nonan-7-yl)-N-((3-nitro-4-(((tetrahydro-2H-pyran-4-yl)methyl)amino)phenyl)sulfonyl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((R)-3-(o-tolyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)-4-(2-((R)-3-(o-tolyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)-4-(2-((R)-3-(o-tolyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)-4-(2-((R)-3-(o-tolyl)morpholino)-7-azaspiro[3.5]nonan-7-yl)benzamide,
2-((S)-1,2,4,4a,5,6-hexahydro-[1,4]oxazino[4,3-d]pyrrolo[3′,2′:5,6]pyrido[3,2-b][1,4]diazepin-7(11H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((R)-1,2,4,4a,5,6-hexahydro-[1,4]oxazino[4,3-d]pyrrolo[3′,2′:5,6]pyrido[3,2-b][1,4]diazepin-7(11H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((5aR,9aS)-6,7,9a,10-tetrahydro-1H,9H-pyrano[4,3-e]pyrrolo[3′,2′:5,6]pyrido[2,3-b][1,4]oxazepin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((5aS,9aR)-6,7,9a,10-tetrahydro-1H,9H-pyrano[4,3-e]pyrrolo[3′,2′:5,6]pyrido[2,3-b][1,4]oxazepin-5(5aH)-yl)benzamide,
2-((R)-2,3,5,5a,6,7-hexahydro-1H-pyrrolo[3″,2″:5′,6′]pyrido[2′,3′:2,3][1,4]diazepino[7,1-c][1,4]oxazepin-8(12H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((S)-2,3,5,5a,6,7-hexahydro-1H-pyrrolo[3″,2″:5′,6′]pyrido[2′,3′:2,3][1,4]diazepino[7,1-c][1,4]oxazepin-8(12H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,13aR)-2,3,5,5a,13,13a-hexahydro-[1,4]dioxepino[5,6-f]pyrrolo[3′,2′:5,6]pyrido[2,3-b][1,4]oxazepin-12(8H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aR,13aS)-2,3,5,5a,13,13a-hexahydro-[1,4]dioxepino[5,6-f]pyrrolo[3′,2′:5,6]pyrido[2,3-b][1,4]oxazepin-12(8H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aR,13aR)-2,3,5,5a,13,13a-hexahydro-[1,4]dioxepino[6,5-e]pyrrolo[3′,2′:5,6]pyrido[2,3-b][1,4]oxazepin-6(10H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,13aS)-2,3,5,5a,13,13a-hexahydro-[1,4]dioxepino[6,5-e]pyrrolo[3′,2′:5,6]pyrido[2,3-b][1,4]oxazepin-6(10H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
4-(2-((S)-2,3-dihydrospiro[indene-1,2′-pyrrolidin]-1′-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((R)-3,3-dimethyl-2,3-dihydrospiro[indene-1,3′-morpholin]-4′-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-1,1-dimethylspiro[isochromane-4,3′-morpholin]-4′-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1R,3S)-3-methyl-2,3-dihydrospiro[indene-1,3′-morpholin]-4′-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1R,3R)-3-methyl-2,3-dihydrospiro[indene-1,3′-morpholin]-4′-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2H-spiro[benzofuran-3,2′-piperidin]-1′-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-((S)-2′H-spiro[azepane-2,3′-benzofuran]-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((R)-spiro[chromane-4,2′-pyrrolidin]-1′-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((R)-spiro[azepane-2,4′-chroman]-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-(3,4-dihydro-2H-spiro[benzo[b]oxepine-5,2′-pyrrolidin]-1′-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-(3,4-dihydro-2H-spiro[benzo[b]oxepine-5,3′-morpholin]-4′-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
4-(2-(3′,4′-dihydro-2′H-spiro[azepane-2,5′-benzo[b]oxepin]-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-(((4-fluorotetrahydro-2H-pyran-4-yl)methyl)amino)-3-nitrophenyl)sulfonyl)-4-(2-((R)-spiro[chromane-4,2′-piperidin]-1′-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-(((1,4-dioxan-2-yl)methyl)amino)-3-nitrophenyl)sulfonyl)-4-(2-((R)-spiro[chromane-4,2′-piperidin]-1′-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-spiro[chromane-4,2′-piperidin]-1′-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((R)-spiro[chromane-4,2′-piperidin]-1′-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-spiro[chromane-4,2′-piperidin]-1′-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)-2-((5aR,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)-2-((5aS,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)-2-((5aR,9aS)-5a,8,9,9a-tetrahydro-6H-pyrano[4,3-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)-2-((5aS,9aR)-5a,8,9,9a-tetrahydro-6H-pyrano[4,3-b]pyrrolo[3′,2′:5,6]pyrido[3,2-e][1,4]oxazin-5(1H)-yl)benzamide,
2-((6aS,10aR)-6,6a,7,8,10,10a-hexahydropyrano[4,3-f]pyrrolo[3′,2′:5,6]pyrido[2,3-b][1,4]oxazepin-5(1H)-yl)-N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)benzamide,
2-((6aR,10aS)-6,6a,7,8,10,10a-hexahydropyrano[4,3-f]pyrrolo[3′,2′:5,6]pyrido[2,3-b][1,4]oxazepin-5(1H)-yl)-N-((3-nitro-4-(((tetrahydro-2H-pyran-4-yl)methyl)amino)phenyl)sulfonyl)-4-(2-((R)-spiro[chromane-4,2′-piperidin]-1′-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(4-((S)-5-(2-isopropylphenyl)-7-oxa-2-azaspiro[3.5]nonan-2-yl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((R)-5-(2-isopropylphenyl)-7-oxaspiro[3.5]nonan-2-yl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((3S,4S)-3-(2-isopropylphenyl)tetrahydro-2H-pyran-4-yl)-2,7-diazaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((3S,4R)-3-(2-isopropylphenyl)tetrahydro-2H-pyran-4-yl)-2,7-diazaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1S,2S)-2-(2-isopropylphenyl)cyclohexyl)-2,7-diazaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1R,2S)-2-(2-isopropylphenyl)cyclohexyl)-2,7-diazaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-4-(2-isopropylphenyl)morpholin-3-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-(2-isopropylphenyl)morpholin-3-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(7-((R)-3-(2-isopropylphenyl)morpholino)-2-azaspiro[3.5]nonan-2-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(6-((R)-3-(2-isopropylphenyl)morpholino)-2-azaspiro[3.3]heptan-2-yl)benzamide,
4-(2-((R)-3,3-dimethyl-2,3-dihydrospiro[indene-1,3′-morpholin]-4′-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
4-(2-((R)-1,1-dimethylspiro[isochromane-4,3′-morpholin]-4′-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((1R,3S)-3-methyl-2,3-dihydrospiro[indene-1,3′-morpholin]-4′-yl)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((1R,3R)-3-methyl-2,3-dihydrospiro[indene-1,3′-morpholin]-4′-yl)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(4-((S)-5-(2-isopropylphenyl)-7-oxa-2-azaspiro[3.5]nonan-2-yl)piperidin-1-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(4-((R)-5-(2-isopropylphenyl)-7-oxaspiro[3.5]nonan-2-yl)piperazin-1-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((3S,4S)-3-(2-isopropylphenyl)tetrahydro-2H-pyran-4-yl)-2,7-diazaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((3S,4R)-3-(2-isopropylphenyl)tetrahydro-2H-pyran-4-yl)-2,7-diazaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((1S,2S)-2-(2-isopropylphenyl)cyclohexyl)-2,7-diazaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((1R,2S)-2-(2-isopropylphenyl)cyclohexyl)-2,7-diazaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((S)-4-(2-isopropylphenyl)morpholin-3-yl)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-4-(2-isopropylphenyl)morpholin-3-yl)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-isopropylphenyl)morpholino)-7-azaspiro[3.5]nonan-7-yl)-N-((3-nitro-4-(((tetrahydro-2H-pyran-4-yl)methyl)amino)phenyl)sulfonyl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((3-nitro-4-(((tetrahydro-2H-pyran-4-yl)methyl)amino)phenyl)sulfonyl)-4-(2-((R)-3-(o-tolyl)morpholino)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-isopropoxyphenyl)morpholino)-7-azaspiro[3.5]nonan-7-yl)-N-((3-nitro-4-(((tetrahydro-2H-pyran-4-yl)methyl)amino)phenyl)sulfonyl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropoxyphenyl)morpholino)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(6-((R)-3-(2-isopropylphenyl)morpholino)-2-azaspiro[3.3]heptan-2-yl)benzamide,
2-((S)-8-fluoro-1,2,4a,5-tetrahydro-4H-pyrrolo[3″,2″:5′,6′]pyrido[3′,2′:5,6]pyrazino[2,1-c][1,4]oxazin-6(10H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((R)-8-fluoro-1,2,4a,5-tetrahydro-4H-pyrrolo[3″,2″:5′,6′]pyrido[3′,2′:5,6]pyrazino[2,1-c][1,4]oxazin-6(10H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((S)-9-fluoro-2,3,5a,6-tetrahydro-1H,5H-pyrrolo[3″,2″:5′,6′]pyrido[3′,2′:5,6]pyrazino[2,1-c][1,4]oxazepin-7(11H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((R)-9-fluoro-2,3,5a,6-tetrahydro-1H,5H-pyrrolo[3″,2″:5′,6′]pyrido[3′,2′:5,6]pyrazino[2,1-c][1,4]oxazepin-7(11H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aR,10aR)-3-fluoro-5a,6,7,9,10,10a-hexahydrooxepino[4,5-b]pyrrolo[3′,2′:5,6]pyrido[3,2-e][1,4]oxazin-5(1H)-yl)-4-(2-((R)-3-(2-isopropylphenyl)morpholino)-7-azaspiro[3.5]nonan-7-yl)-N-((3-nitro-4-(((tetrahydro-2H-pyran-4-yl)methyl)amino)phenyl)sulfonyl)benzamide,
2-((5aS,10aS)-3-fluoro-5a,6,7,9,10,10a-hexahydrooxepino[4,5-b]pyrrolo[3′,2′:5,6]pyrido[3,2-e][1,4]oxazin-5(1H)-yl)-4-(2-((R)-3-(2-isopropylphenyl)morpholino)-7-azaspiro[3.5]nonan-7-yl)-N-((3-nitro-4-(((tetrahydro-2H-pyran-4-yl)methyl)amino)phenyl)sulfonyl)benzamide,
2-((6aR,10aR)-3-fluoro-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)benzamide,
2-((6aS,10aS)-3-fluoro-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)benzamide,
2-((6aS,10aS)-3-fluoro-6a,9,10,10a-tetrahydro-1H,7H-pyrano[3,4-f]pyrrolo[3′,2′:5,6]pyrido[2,3-b][1,4]oxazepin-5(6H)-yl)-N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)benzamide,
2-((6aR,10aR)-3-fluoro-6a,9,10,10a-tetrahydro-1H,7H-pyrano[3,4-f]pyrrolo[3′,2′:5,6]pyrido[2,3-b][1,4]oxazepin-5(6H)-yl)-N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)benzamide,
2-((S)-9-fluoro-1,2,4,4a,5,6-hexahydro-[1,4]oxazino[4,3-d]pyrrolo[3′,2′:5,6]pyrido[3,2-b][1,4]diazepin-7(11H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((R)-9-fluoro-1,2,4,4a,5,6-hexahydro-[1,4]oxazino[4,3-d]pyrrolo[3′,2′:5,6]pyrido[3,2-b][1,4]diazepin-7(11H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aR,9aS)-3-fluoro-6,7,9a,10-tetrahydro-1H,9H-pyrano[4,3-e]pyrrolo[3′,2′:5,6]pyrido[2,3-b][1,4]oxazepin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,9aR)-3-fluoro-6,7,9a,10-tetrahydro-1H,9H-pyrano[4,3-e]pyrrolo[3′,2′:5,6]pyrido[2,3-b][1,4]oxazepin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((R)-10-fluoro-2,3,5,5a,6,7-hexahydro-1H-pyrrolo[3″,2″:5′,6′]pyrido[2′,3′:2,3][1,4]diazepino[7,1-c][1,4]oxazepin-8(12H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((S)-10-fluoro-2,3,5,5a,6,7-hexahydro-1H-pyrrolo[3″,2″:5′,6′]pyrido[2′,3′:2,3][1,4]diazepino[7,1-c][1,4]oxazepin-8(12H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,13aR)-10-fluoro-2,3,5,5a,13,13a-hexahydro-[1,4]dioxepino[5,6-f]pyrrolo[3′,2′:5,6]pyrido[2,3-b][1,4]oxazepin-12(8H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aR,13aS)-10-fluoro-2,3,5,5a,13,13a-hexahydro-[1,4]dioxepino[5,6-f]pyrrolo[3′,2′:5,6]pyrido[2,3-b][1,4]oxazepin-12(8H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aR,13aR)-8-fluoro-2,3,5,5a,13,13a-hexahydro-[1,4]dioxepino[6,5-e]pyrrolo[3′,2′:5,6]pyrido[2,3-b][1,4]oxazepin-6(10H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,13aS)-8-fluoro-2,3,5,5a,13,13a-hexahydro-[1,4]dioxepino[6,5-e]pyrrolo[3′,2′:5,6]pyrido[2,3-b][1,4]oxazepin-6(10H)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)-4-(4-methoxybenzyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(2-isopropylphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-cyclopropyl-3-methoxybenzyl)-2-(2-isopropylphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)-4-((7-methoxy-2-methylbenzofuran-5-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-2-(2-isopropylphenyl)-4-(4-methoxybenzyl)piperazin-1-yl)-7-azaspiro[3.5]nonan-7-yl)benzamide,
4-(2-((R)-4-(3,4-difluorobenzyl)-2-(2-isopropylphenyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
4-(2-((R)-4-(4-cyclopropyl-3-methoxybenzyl)-2-(2-isopropylphenyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-2-(2-isopropylphenyl)-4-((7-methoxy-2-methylbenzofuran-5-yl)methyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(6-((R)-2-(2-isopropylphenyl)-4-(4-methoxybenzyl)piperazin-1-yl)-2-azaspiro[3.3]heptan-2-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(6-((R)-4-(3,4-difluorobenzyl)-2-(2-isopropylphenyl)piperazin-1-yl)-2-azaspiro[3.3]heptan-2-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(6-((R)-4-(4-cyclopropyl-3-methoxybenzyl)-2-(2-isopropylphenyl)piperazin-1-yl)-2-azaspiro[3.3]heptan-2-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(6-((R)-2-(2-isopropylphenyl)-4-((7-methoxy-2-methylbenzofuran-5-yl)methyl)piperazin-1-yl)-2-azaspiro[3.3]heptan-2-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(6-((R)-2-(2-isopropylphenyl)-4-(4-methoxybenzyl)piperazin-1-yl)-2-azaspiro[3.3]heptan-2-yl)benzamide,
4-(6-((R)-4-(3,4-difluorobenzyl)-2-(2-isopropylphenyl)piperazin-1-yl)-2-azaspiro[3.3]heptan-2-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
4-(6-((R)-4-(4-cyclopropyl-3-methoxybenzyl)-2-(2-isopropylphenyl)piperazin-1-yl)-2-azaspiro[3.3]heptan-2-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(6-((R)-2-(2-isopropylphenyl)-4-((7-methoxy-2-methylbenzofuran-5-yl)methyl)piperazin-1-yl)-2-azaspiro[3.3]heptan-2-yl)benzamide,
N-((4-((((1r,4r)-4-hydroxy-4-methylcyclohexyl)methyl)amino)-3-nitrophenyl)sulfonyl)-4-(2-((R)-3-(4-isopropylthiophen-3-yl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-3-(3-isopropylthiophen-2-yl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((R)-3-(2-isopropylthiophen-3-yl)morpholino)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-((2R,4S,6R)-1,1,6-trimethyl-2-((R)-3-(o-tolyl)-1,4-oxazepan-4-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-((2S,4R,6R)-1,1,6-trimethyl-2-((R)-3-(o-tolyl)-1,4-oxazepan-4-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-((1S,2R,4S,6R)-2-((R)-3-(2-isopropylphenyl)morpholino)-1,6-dimethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(o-tolyl)-1,4-oxazepan-4-yl)-7-azaspiro[3.5]nonan-7-yl)benzamide,
2-((5aS,9aR)-9,9-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(o-tolyl)-1,4-oxazepan-4-yl)-7-azaspiro[3.5]nonan-7-yl)benzamide,
2-((5aS,9aR)-3-fluoro-9,9-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(o-tolyl)-1,4-oxazepan-4-yl)-7-azaspiro[3.5]nonan-7-yl)benzamide,
2-((5aS,9aR)-7,7-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(o-tolyl)-1,4-oxazepan-4-yl)-7-azaspiro[3.5]nonan-7-yl)benzamide,
2-((5aS,9aR)-3-fluoro-7,7-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(o-tolyl)-1,4-oxazepan-4-yl)-7-azaspiro[3.5]nonan-7-yl)benzamide,
2-((5aS,9aR)-9,9-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,9aR)-7,7-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,9aR)-3-fluoro-9,9-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,9aR)-3-fluoro-7,7-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N-((4-(((4-fluorotetrahydro-2H-pyran-4-yl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((S)-3-((1r,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((S)-3-((1s,4R)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((S)-3-((1s,4R)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((R)-3-((1r,4R)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N—(((S)-3-((1r,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((3R,5R)-5-methyl-3-(o-tolyl)-1,4-oxazepan-4-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-5,5-dimethyl-3-(o-tolyl)-1,4-oxazepan-4-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-6,6-dimethyl-3-(o-tolyl)-1,4-oxazepan-4-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-7,7-dimethyl-3-(o-tolyl)-1,4-oxazepan-4-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)-4,4-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-6-(2-isopropylphenyl)-2,2-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)-5,5-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-hydroxy-2-(2-isopropylphenyl)-4-methylpiperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-hydroxy-2-(2-isopropylphenyl)-4-methylpiperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5S)-5-hydroxy-2-(2-isopropylphenyl)-5-methylpiperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5R)-5-hydroxy-2-(2-isopropylphenyl)-5-methylpiperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4,4-difluoro-2-(2-isopropylphenyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-5,5-difluoro-2-(2-isopropylphenyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4R)-2-(2-isopropylphenyl)-4-methoxy-4-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4S)-2-(2-isopropylphenyl)-4-methoxy-4-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4R)-2-(2-isopropylphenyl)-4-methyl-4-(p-tolyloxy)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4S)-2-(2-isopropylphenyl)-4-methyl-4-(p-tolyloxy)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-isopropyl-2-(2-isopropylphenyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)-4-(isopropylsulfonyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)-1,1-dioxidothiomorpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-ethynylphenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-(1,1,1-trifluoro-2-methylpropan-2-yl)phenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-3-(2-(2-hydroxypropan-2-yl)phenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-(2-aminopropan-2-yl)phenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-(trifluoromethoxy)phenyl)morpholino)-7-azaspiro[3.5]nonan-7-yl)benzamide,
4-(2-((R)-3-(2-cyclopropoxyphenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-(tert-butoxy)phenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-cyclobutoxyphenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1R,4s)-4-fluoro-4-((S)-7-(N-(2-((5aS,9aR)-3-fluoro-7,7-dimethyl-6,7,9,9a-tetrahydro-1H-pyrano[3,4-b]pyrrolo[3′,2′:5,6]pyrido[3,2-e][1,4]oxazin-5(5aH)-yl)-4-((R)-2-((R)-5-(2-isopropylphenyl)-3,3-dimethylmorpholino)-6-methyl-7-azaspiro[3.5]nonan-7-yl)benzoyl)sulfamoyl)-5-nitro-3,4-dihydro-2H-benzo[b][1,4]oxazin-3-yl)-1-methylcyclohexyl dihydrogen phosphate,
N-((4-((((1r,4r)-4-hydroxy-4-methylcyclohexyl)methyl)amino)-3-nitrophenyl)sulfonyl)-2-((5aS,8S,9aS)-8-hydroxy-8-methyl-5a,6,7,8,9,9a-hexahydrobenzo[b]pyrrolo[3′,2′:5,6]pyrido[3,2-e][1,4]oxazin-5(1H)-yl)-4-(2-((R)-3-(2-isopropylphenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((5aR,8S,9aR)-8-hydroxy-8-methyl-5a,6,7,8,9,9a-hexahydrobenzo[b]pyrrolo[3′,2′:5,6]pyrido[3,2-e][1,4]oxazin-5(1H)-yl)-4-(2-((R)-3-(2-isopropylphenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((5aS,8R,9aS)-8-hydroxy-8-methyl-5a,6,7,8,9,9a-hexahydrobenzo[b]pyrrolo[3′,2′:5,6]pyrido[3,2-e][1,4]oxazin-5(1H)-yl)-4-(2-((R)-3-(2-isopropylphenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((5aR,8R,9aR)-8-hydroxy-8-methyl-5a,6,7,8,9,9a-hexahydrobenzo[b]pyrrolo[3′,2′:5,6]pyrido[3,2-e][1,4]oxazin-5(1H)-yl)-4-(2-((R)-3-(2-isopropylphenyl)morpholino)-7-azaspiro[3.5]nonan-7-yl)benzamide,
4-(2-((S)-4,4-dimethyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-5,5-dimethyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2,2-dimethyl-6-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-(2-((R)-3-(2-isopropylphenyl)morpholino)cyclopropyl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((S)-1-((R)-3-(2-isopropylphenyl)morpholino)-6-azaspiro[2.5]octan-6-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((1S,3R)-3-((R)-3-(2-isopropylphenyl)morpholino)cyclobutyl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((1R,3S)-3-((R)-3-(2-isopropylphenyl)morpholino)cyclobutyl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((1S,4R)-4-((R)-3-(2-isopropylphenyl)morpholino)cyclohexyl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((1R,4S)-4-((R)-3-(2-isopropylphenyl)morpholino)cyclohexyl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((((1r,4r)-4-hydroxy-4-methylcyclohexyl)methyl)amino)-N-((4-(2-((R)-3-(2-isopropylphenyl)morpholino)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)phenyl)sulfonyl)-3-nitrobenzamide,
4-((((1r,4r)-4-hydroxy-4-methylcyclohexyl)methyl)amino)-3-nitro-N-((2-((5aR,9aS)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)phenyl)sulfonyl)benzamide,
4-((((1r,4r)-4-hydroxy-4-methylcyclohexyl)methyl)amino)-N-((4-(2-((R)-3-(2-isopropoxyphenyl)morpholino)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)phenyl)sulfonyl)-3-nitrobenzamide,
N-((2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-isopropylphenyl)morpholino)-7-azaspiro[3.5]nonan-7-yl)phenyl)sulfonyl)-4-((((1r,4r)-4-hydroxy-4-methylcyclohexyl)methyl)amino)-3-nitrobenzamide,
N-((4-(2-((R)-3-(2-isopropylphenyl)morpholino)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)phenyl)sulfonyl)-3-nitro-4-(((tetrahydro-2H-pyran-4-yl)methyl)amino)benzamide,
N-((5-((((1r,4r)-4-hydroxy-4-methylcyclohexyl)methyl)amino)-6-nitropyridin-2-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((6-((((1r,4r)-4-hydroxy-4-methylcyclohexyl)methyl)amino)-5-nitropyridazin-3-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((6-((((1r,4r)-4-hydroxy-4-methylcyclohexyl)methyl)amino)-5-nitropyridin-3-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((5-((((1r,4r)-4-hydroxy-4-methylcyclohexyl)methyl)amino)-4-nitropyridin-2-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((S)-2-((1r,4S)-1-fluoro-4-hydroxy-4-methylcyclohexyl)-8-nitro-2,3-dihydro-1H-pyrido[2,3-b][1,4]oxazin-6-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-2-((1r,4R)-1-fluoro-4-hydroxy-4-methylcyclohexyl)-8-nitro-2,3-dihydro-1H-pyrido[2,3-b][1,4]oxazin-6-yl)sulfonyl)-4-(2-((R)-3-(2-isopropylphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((6-((((1r,4r)-4-hydroxy-4-methylcyclohexyl)methyl)amino)-5-nitropyridin-3-yl)sulfonyl)-4-(2-((R)-3-(2-isopropylphenyl)morpholino)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((5-((((1r,4r)-4-hydroxy-4-methylcyclohexyl)methyl)amino)-4-nitropyridin-2-yl)sulfonyl)-4-(2-((R)-3-(2-isopropylphenyl)morpholino)-7-azaspiro[3.5]nonan-7-yl)benzamide,
N—(((S)-2-((1r,4S)-1-fluoro-4-hydroxy-4-methylcyclohexyl)-8-nitro-2,3-dihydro-1H-pyrido[2,3-b][1,4]oxazin-6-yl)sulfonyl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-isopropylphenyl)morpholino)-7-azaspiro[3.5]nonan-7-yl)benzamide,
N—(((R)-2-((1r,4R)-1-fluoro-4-hydroxy-4-methylcyclohexyl)-8-nitro-2,3-dihydro-1H-pyrido[2,3-b][1,4]oxazin-6-yl)sulfonyl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-isopropylphenyl)morpholino)-7-azaspiro[3.5]nonan-7-yl)benzamide,
2-((5aS,9aR)-1,6,7,9,9a,10-hexahydropyrano[3,4-b]pyrrolo[3′,2′:5,6]pyrido[3,2-e]pyrazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-2-((5aS,9aR)-10-isopropyl-1,6,7,9,9a,10-hexahydropyrano[3,4-b]pyrrolo[3′,2′:5,6]pyrido[3,2-e]pyrazin-5(5aH)-yl)-4-(2-((R)-3-(2-isopropylphenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((5aS,9aR)-10-(oxetan-3-yl)-1,6,7,9,9a,10-hexahydropyrano[3,4-b]pyrrolo[3′,2′:5,6]pyrido[3,2-e]pyrazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((5aS,9aR)-10-(tetrahydro-2H-pyran-4-yl)-1,6,7,9,9a,10-hexahydropyrano[3,4-b]pyrrolo[3′,2′:5,6]pyrido[3,2-e]pyrazin-5(5aH)-yl)benzamide,
2-((5aS,9aR)-10-(4-hydroxy-4-methylcyclohexyl)-1,6,7,9,9a,10-hexahydropyrano[3,4-b]pyrrolo[3′,2′:5,6]pyrido[3,2-e]pyrazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
2-((5aS,9aR)-10-(4-hydroxy-4-methylcyclohexyl)-1,6,7,9,9a,10-hexahydropyrano[3,4-b]pyrrolo[3′,2′:5,6]pyrido[3,2-e]pyrazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)benzamide,
((5aS,9aR)-5-(2-(((4-((((1r,4r)-4-hydroxy-4-methylcyclohexyl)methyl)amino)-3-nitrophenyl)sulfonyl)carbamoyl)-5-(2-((R)-3-(2-isopropylphenyl)morpholino)-7-azaspiro[3.5]nonan-7-yl)phenyl)-5,5a,6,7,9,9a-hexahydro-11H-pyrano[3,4-b]pyrrolo[3′,2′:5,6]pyrido[3,2-e][1,4]oxazin-11-yl)methyl dihydrogen phosphate

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration, and both of these configurations are within the scope of the invention.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof.

The term “geometric isomer” refers to cyclic compounds having at least two substituents, wherein the two substituents are both on the same side of the ring (cis) or wherein the substituents are each on opposite sides of the ring (trans). When a disclosed compound is named or depicted by structure without indicating stereochemistry, it is understood that the name or the structure encompasses one or more of the possible stereoisomers, or geometric isomers, or a mixture of the encompassed stereoisomers or geometric isomers.

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geomeric isomers in the mixture.

Racemic mixture means 50% of one enantiomer and 50% of is corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures of one or more diastereomers (e.g., racemic mixtures) of the compound.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers also can be obtained from diastereomerically- or enantiomerically-pure intermediates, reagents, and catalysts by well-known asymmetric synthetic methods.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Prodrug derivatives: prodrugs, upon administration to a subject, will converted in vivo into active compounds of the present invention [Nature Reviews of Drug Discovery, 2008, Volume 7, p255]. It is noted that in many instances, the prodrugs themselves also fall within the scope of the range of compounds according to the present invention. The prodrugs of the compounds of the present invention can be prepared by standard organic reaction, for example, by reacting with a carbamylating agent (e.g., 1,1-acyloxyalkylcarbonochloridate, para-nitrophenyl carbonate, or the like) or an acylating agent. Further examples of methods and strategies of making prodrugs are described in Bioorganic and Medicinal Chemistry Letters, 1994, Vol. 4, p. 1985.
Deuterium-enriched compounds: deuterium (D or ²H) is a stable, non-radioactive isotope of hydrogen and has an atomic weight of 2.0144. Hydrogen naturally occurs as a mixture of the isotopes ^{X}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. The invention encompasses any pharmaceutically acceptable salts and solvates of any one of the above-described compounds and modifications thereof. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The compounds disclosed therein are bcl-2 inhibitors. The pharmaceutical composition of the present invention comprises one or more bcl-2 inhibitors, or a pharmaceutically acceptable salt thereof, and a pharmaceutically acceptable carrier or diluent.

“Pharmaceutically acceptable carrier” and “pharmaceutically acceptable diluent” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The pharmaceutical compositions of the present invention optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

The pharmaceutical compositions of the present invention may further comprise other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions of the present invention may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-α-tocopheryl PEG 1000 succinate (20-50%), using complexation such as HPβCD and SBEβCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀, C₁-C₆). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀, C₂-C₆) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀, C₂-C₆) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl. The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

The term “heterocycloalkyl” refers to a saturated or unsaturated nonaromatic monocyclic, bicyclic, tricyclic, or tetracyclic system having one or more heteroatoms (such as O, N, S, B, P, Si, or Se), which may be the same or different. Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic monocyclic, bicyclic, tricyclic, or tetracyclic ring system having one or more heteroatoms (such as O, N, S, P, B, Si, or Se) and one or more double bonds.

Spiroalkyl refers to a compound comprising two saturated cyclic alkyl rings sharing only one common atom (also known as a spiro atom), with no heteroatom and no unsaturated bonds on any of the rings. In one embodiment, the spiroalkyl is bicyclic. In another embodiment, the spiroalikyl has more than two cycles. In certain embodiments, the spiroalkyl compound is a polyspiro compound connected by two or more spiroatoms making up three or more rings. In certain embodiments, one of the rings of the bicyclic spiroalkyl has 3, 4, 5, 6, 7, or 8 atoms, including the common spito atom. In one embodiment, the spiroalkyl is a 5 to 20 membered, 5 to 14 membered, or 5 to 10 membered polycyclic spiroalkyl group. Representative examples of spiroalkyl include, but are not limited to the following groups:

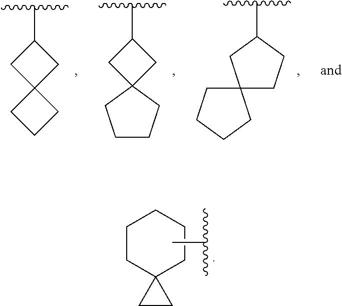

Spiroheterocyclyl refers to a compound comprising two non-saturated rings sharing only one common atom (also known as a spiro atom), with at least one heteroatom on one of the two rings, such as a polycyclic heterocyclyl group with rings connected through one common carbon atom. The common atom can be carbon (C), silicon, or nitrogen (such as a positively charged quaternary nitrogen atom). The heteroatoms can comprise nitrogen, quaternary nitrogen, oxidized nitrogen (e.g., NO), oxygen, silicon, and sulfur, including sulfoxide and sulfone, and the remaining ring atoms are C. In addition, one or more of the rings may contain one or more double bonds. In one embodiment, the spiro heterocyclyl is bicyclic, with heteroatom(s) on either one or both cycles. In certain embodiments, one of the rings of the bicyclic spiro heterocyclyl has 3, 4, 5, 6, 7, or 8 atoms, including the common spito atom. In certain embodiments, the spiro heterocyclic compound is a polyspiro compound connected by two or more spiroatoms making up three or more rings. In one embodiment, the spiro heterocyclyl is a 5 to 20 membered, 5 to 14 membered, or 5 to 10 membered polycyclic heterocyclyl group. Representative examples of spiro heterocyclyl include, but are not limited to the following groups:

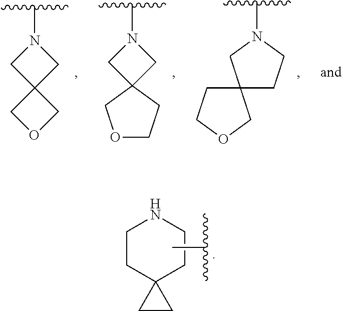

Fused heterocyclyl refers to a polycyclic heterocyclyl group, wherein each ring in the group shares an adjacent pair of atoms (such as carbon atoms) with another ring in the group, wherein one or more rings can contain one or more double bonds, and wherein said rings have one or more heteroatoms, which can be nitrogen, quaternary nitrogen, oxidized nitrogen (e.g., NO), oxygen, and sulfur, including sulfoxide and sulfone, and the remaining ring atoms are C. In certain embodiments, the fused heterocyclyl is bicyclic. In certain embodiments, the fused heterocyclyl contains more than two rings, at least two of which share an adjacent pair of atoms. In one embodiment, the fused heterocyclyl is a 5 to 20 membered, 5 to 12 membered, or 5 to 10 membered polycyclic heterocyclyl group. Representative examples of fused heterocyclyl include, but are not limited to the following groups:

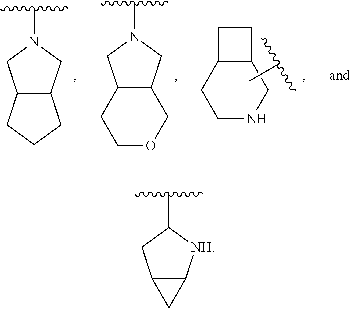

Bridged heterocyclyl refers to a compound having at least two rings sharing three or more common ring atoms, separating the two bridgehead atoms by a bridge containing at least one atom, wherein at least one ring atom is a heteroatom. The bridgehead atoms are the atoms from which three bonds radiate and where the rings meet. The rings of the bridged heterocyclyl can have one or more double bonds, and the ring heteroatom(s) can be nitrogen, quaternary nitrogen, oxidized nitrogen (e.g., NO), oxygen, and sulfur, including sulfoxide and sulfone as ring atoms, while the remaining ring atoms are C. In one embodiment, the bridged heterocyclyl is bicyclic. In one embodiment, the bridged heterocyclyl is a 5 to 20 membered, 5 to 12 membered, or 5 to 10 membered polycyclic heterocyclyl group. Representative examples of bridged heterocyclyl include, but are not limited to the following groups:

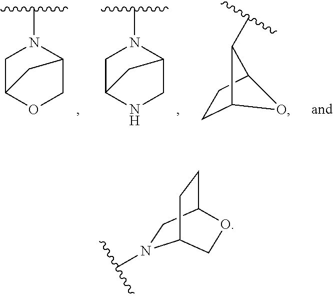

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl.

The term “heteroaryl” refers to an aromatic monocyclic, bicyclic, tricyclic, or tetracylic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, heterocycloalkyl, cycloalkenyl, heterocycloalkenyl, alkylamino, spiroheterocyclyl, fused heterocyclyl, bridged heterocyclyl, aryl, and heteroaryl mentioned above include both substituted and unsubstituted moieties. Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl.

Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group.

Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors(Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKIIa, CAMKIIb, CAMKIId, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CSF1R, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDRNEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TA01, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, KINASE, MUTANTS, ABL1(E255K), ABL1(F3171), ABL1(G250E), ABL1(H396P), ABL1(M351T), ABL1(Q₂₅₂H), ABL1(T3151), ABL1(Y253F), ALK (C1156Y), ALK(L1196M), ALK (F1174L), ALK (R1275Q), BRAF(V599E), BTK(E41K), CHK2(1157T), c-Kit(A829P), c-KIT(D816H), c-KIT(D816V), c-Kit(D820E), c-Kit(N822K), C-Kit (T6701), c-Kit(V559D), c-Kit(V559D/V654A), c-Kit(V559D/T6701), C-Kit (V560G), c-KIT(V654A), C-MET(D1228H), C-MET(D1228N), C-MET(F12001), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R, T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (12020T), LRRK2 (R1441C), p38a(T106M), PDGFRa(D842V), PDGFRa(T6741), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARP), hypoxia-inducible factors(HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc), and other immunotherapies (e.g anti-PD1, anti-PDL1, anti-CTLA4, CAR-T, IDO, A2A antagonist etc).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), antibody conjugate (e.g brentuximab vedotin, ibritumomab tioxetan), cancer immunotherapy such as Interleukin-2, cancer vaccines (e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, anti-metabolites, topoisomerase inhibitors, anti-microtubule agents, kinase inhibitors, epigenetic agents, HSP90 inhibitors, PARP inhibitors, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, pembrolizumab, nivolumab, atezolizumab, durvalumab, avelumab, as tisagenlecleucel, axicabtagene ciloleucel, radiation therapy, or surgery.

The invention further provides methods for the prevention or treatment of a neoplastic disease or autoimmune disease. In one embodiment, the invention relates to a method of treating a neoplastic disease or autoimmune disease, in a subject in need of treatment comprising administering to said subject a therapeutically effective amount of a compound of the invention. In one embodiment, the invention further provides for the use of a compound of the invention in the manufacture of a medicament for halting or decreasing a neoplastic disease or autoimmune disease.

In certain embodiments, the neoplastic disease is a lung cancer, head and neck cancer, central nervous system cancer, prostate cancer, testicular cancer, colorectal cancer, pancreatic cancer, liver cancer, stomach cancer, biliary tract cancer, esophageal cancer, gastrointestinal stromal tumor, breast cancer, cervical cancer, ovarian cancer, uterine cancer, leukemia, lymphomas, multiple myeloma, melanoma, basal cell carcinoma, squamous cell carcinoma, bladder cancer, renal cancer, sarcoma, mesothelioma, thymoma, myelodysplastic syndrome, or myeloproliferative disease.

The autoimmune diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barre syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of schemes. Necessary starting materials may be obtained by standard procedures of organic chemistry. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

A typical approach to synthesize the intermediate

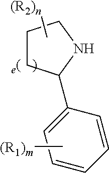

is described in Scheme 1-1 below.

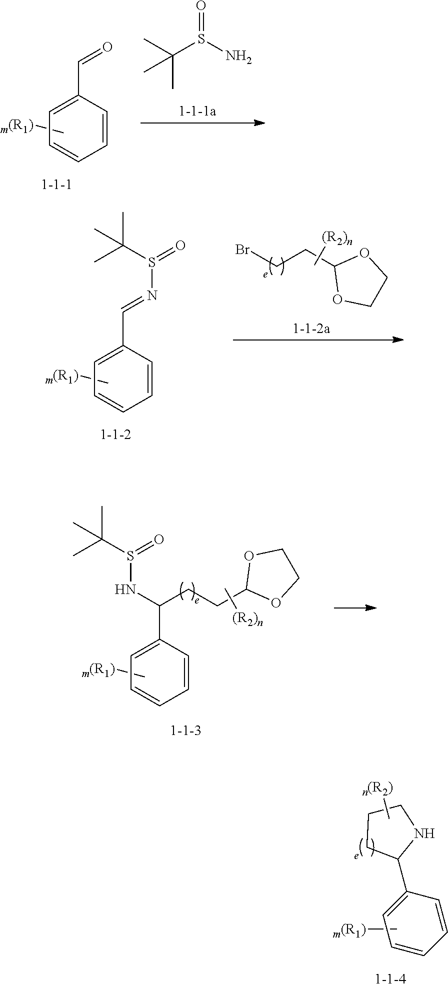

In Scheme 1-1, the condensation of 1-1-1 with the commercially available 1-1-1a results in 1-1-2, and then the reaction of 1-1-2 with 1-1-2a provides 1-1-3. Intramolecular cycliation of 1-1-3 under a suitable condition can give 1.1-4,

Also, the target compounds can be synthesized by alternative methods but not limited to the above procedures.

A typical approach to synthesize the intermediate

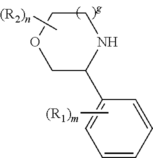

is described in Scheme 1-2 below.

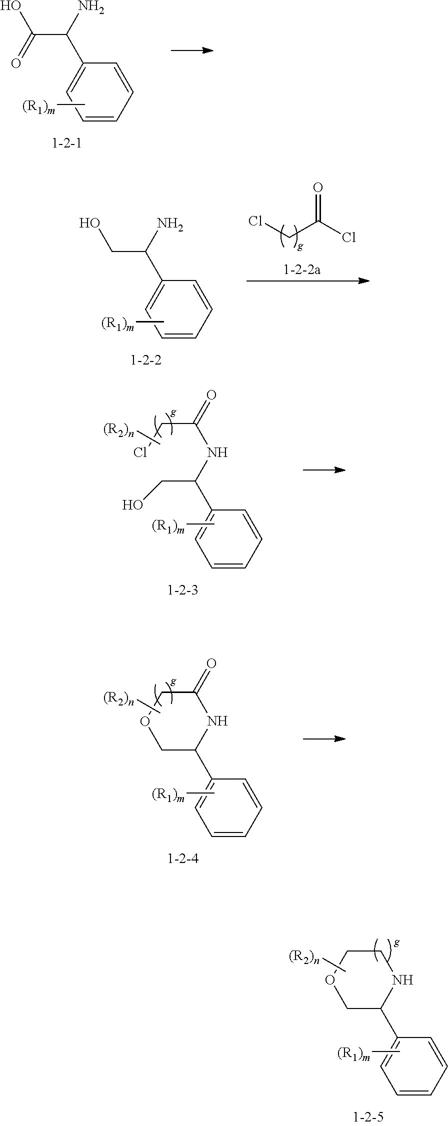

In Scheme 1-2, the starting material 1-2-1 can be prepared by conventional procedures using appropriate compounds and reagents. The starting material 1-2-1 can be reduced to give 1-2-2. After that, the reaction of 1-2-2 with 1-2-2a can afford 1-2-3 readily, which can be converted to 1-2-4 through an intramolecular cyclization reaction. Finally, the intermediate 1-2-4 is reduced to yield the target compounds 1-2-5.

Also, the target compounds can be synthesized by alternative methods but not limited to the above procedures.

A typical approach to synthesize the intermediate

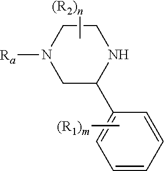

is described in Scheme 1-3 below.

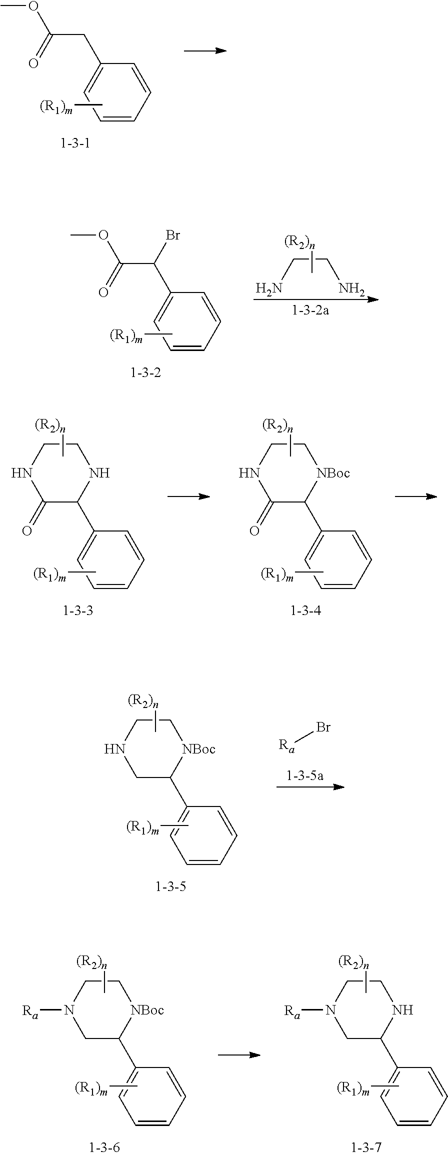

In Scheme 1-3, the starting material 1-3-1 can be prepared by conventional procedures using appropriate compounds and reagents. The bromination of 1-3-1 can generate 1-3-2, which further reacts with 1-3-2a to give 1-3-3. After that, 1-3-3 can be converted to 1-3-4 readily, and then the reduction of 1-3-4 can generate 1-3-5. The intermediate 1-3-5 undergoes an N-substitution reaction with 1-3-5a to give 1-3-6, which is deprotected to afford the intermediate 1-3-7.

Also, the target compounds can be synthesized by alternative methods but not limited to the above procedures.

A typical approach to synthesize the intermediate

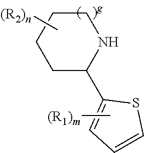

is described in Scheme 1-4 below.

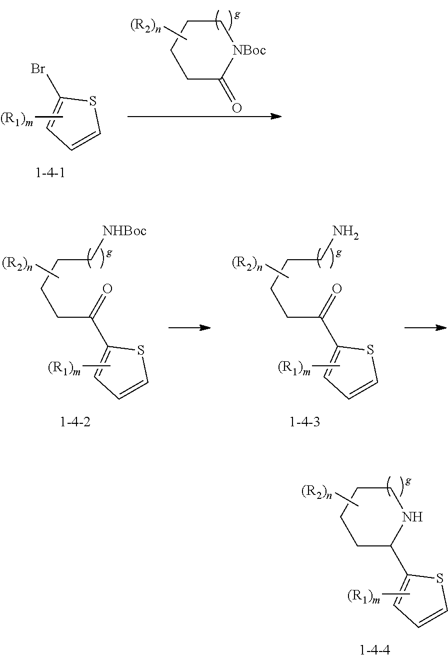

In Scheme 1-4, the starting material 1-4-1 can be prepared by conventional procedures using appropriate compounds and reagents. The starting material 1-4-1 can be converted to 1-4-2 under a literate known condition. Finally, the intermediate 1-4-2 go through a two-step sequence of deprotection reaction and intramolecular reductive amination reaction to generate the target compounds 1-4-4.

Also, the target compounds can be synthesized by alternative methods but not limited to the above procedures.

A typical approach to synthesize the intermediate

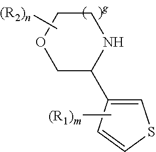

is described in Scheme 1-5 below.

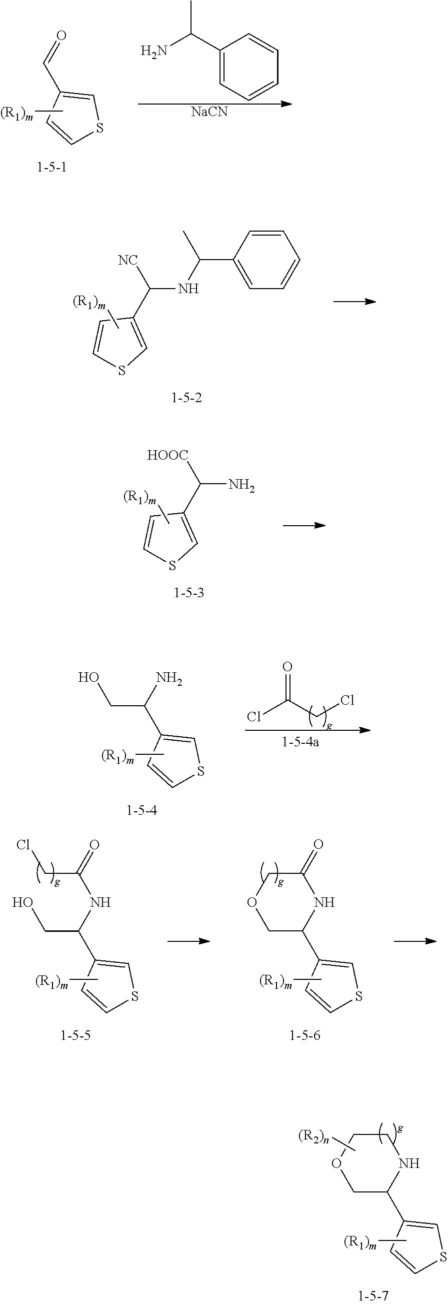

In Scheme 1-5, the starting material 1-5-1 can be prepared by conventional procedures using appropriate compounds and reagents. The starting material 1-5-1 can be converted to 1-5-3 through a two-step sequence of conventional reactions. After that, the carboxylic acid 1-5-3 can be reducted to 1-5-4 readily, which can react with 1-5-4a to give 1-5-5. Finally, 1-5-5 can undergo an intramolecular cyclization to afford 1-5-6, which is further treated with a suitable reducing reagent to afford the target compounds 1-5-7.

Also, the target compounds can be synthesized by alternative methods but not limited to the above procedures.

A typical approach to synthesize the intermediate

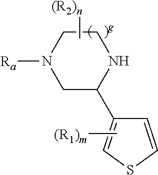

is described in Scheme 1-6 below.

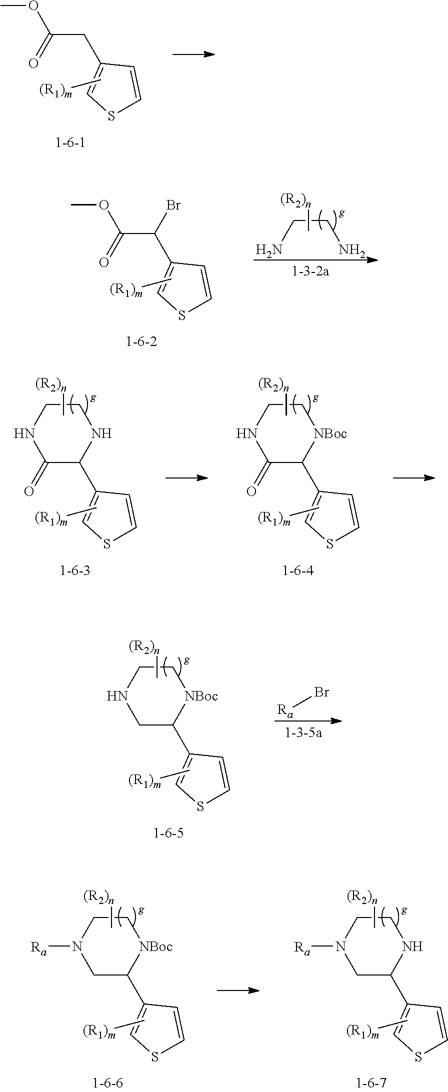

In Scheme 1-6, the starting material 1-6-1 can be prepared by conventional procedures using appropriate compounds and reagents. The bromination of 1-6-1 can generate 1-6-2, which further reacts with 1-3-2a to give 1-6-3. After that, 1-6-3 can be converted to 1-6-4 readily, and then the reduction of 1-6-4 can generate 1-6-5. The intermediate 1-6-5 undergoes an N-substitution reaction with 1-3-5a to give 1-6-6, which can be deprotected to afford the target compounds 1-6-7.

Also, the target compounds can be synthesized by alternative methods but not limited to the above procedures.

A typical approach to synthesize the intermediate

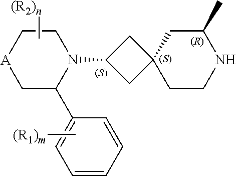

is described in Scheme 1-7

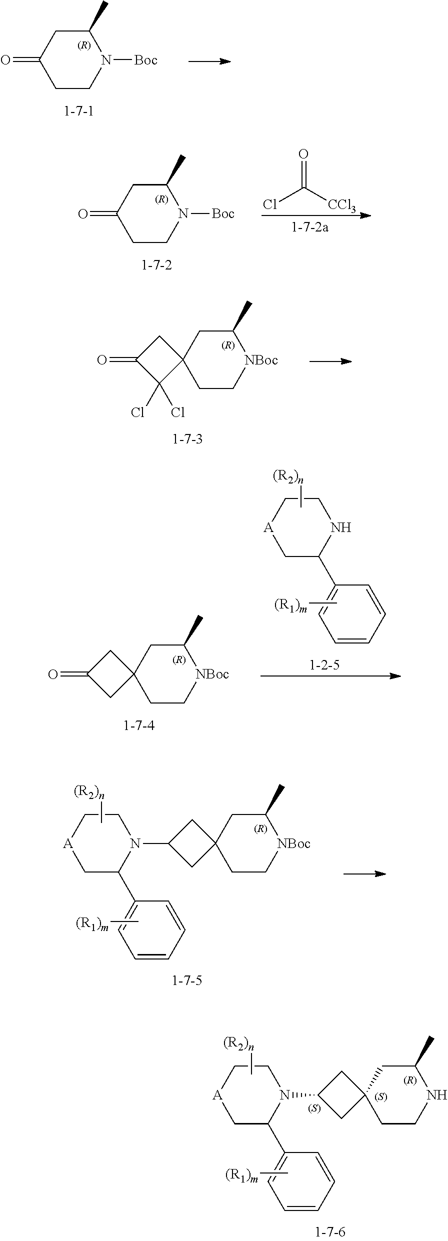

In Scheme 1-7, the starting material 1-7-1 can be prepared by conventional procedures using appropriate compounds and reagents. The starting material 1-7-1 can be converted to 1-7-2 via a general Wittig reaction, which further undergoes Staudinger ketene cycloaddition with 1-7-2a to give 1-7-3. After that, the dichloride 1-7-3 can be reduced to 1-7-4 by a suitable reagent, and then the reaction of 1-7-4 with 1-2-5 can generate 1-7-5. Finally, the deprotection of 1-7-5 can give the target compounds 1-7-6.

Also, the target compounds can be synthesized by alternative methods but not limited to the above procedures.

A typical approach to synthesize the intermediate

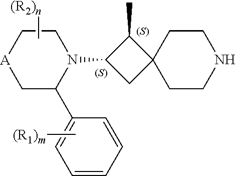

is described in Scheme 1-8

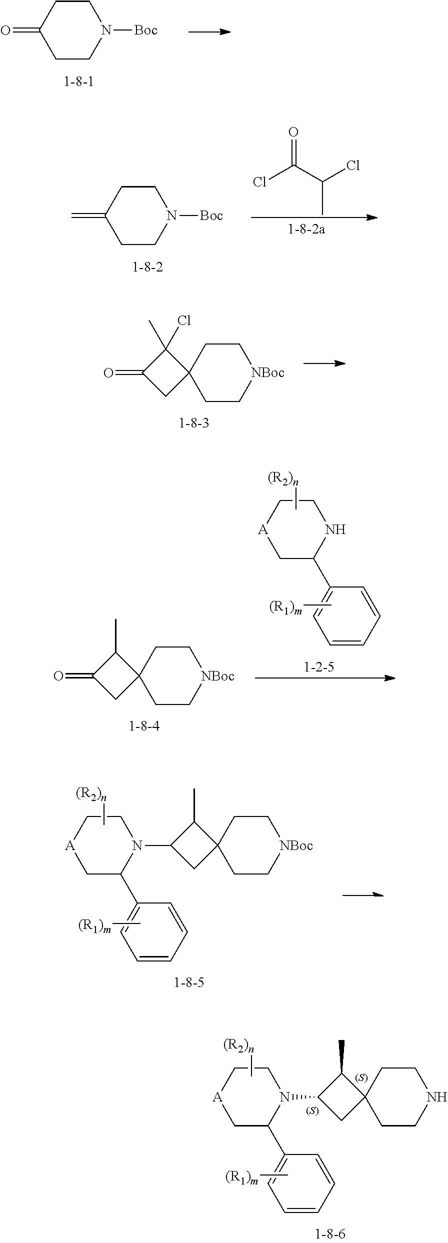

In Scheme 1-8, the starting material 1-8-1 can be prepared by conventional procedures using appropriate compounds and reagents. The starting material 1-8-1 can be converted to 1-8-2 via a general Wittig reaction, which further undergoes Staudinger ketene cycloaddition with 1-8-2a to give 1-8-3. After that, the chloride 1-8-3 can be reducted to 1-8-4 by a suitable reagent, and then the reaction of 1-8-4 with 1-2-5 can generate 1-8-5. Finally, the deprotection of 1-8-5 can give the target compounds 1-8-6.

Also, the target compounds can be synthesized by alternative methods but not limited to the above procedures.

A typical approach to synthesize the intermediate

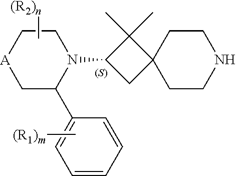

is described in Scheme 1-9 below.

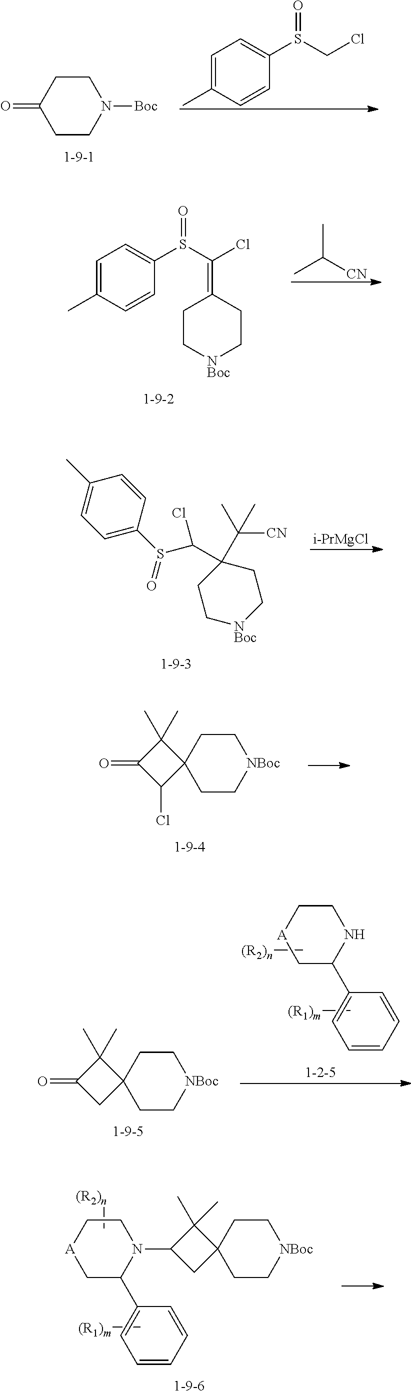

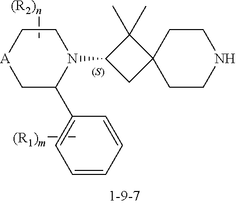

In Scheme 1-9, the starting material 1-9-1 can be prepared by conventional procedures using appropriate compounds and reagents. The starting material 1-9-1 can be converted to 1-9-4 via a sequence of literate known reactions, and then 1-9-4 can be reduced to give 1-9-5. After that, the reductive amination of 1-9-5 with 1-2-5 can generate 1-9-6. Finally, the deprotection of 1-9-6 can give the target compounds 1-9-7.

Also, the target compounds can be synthesized by alternative methods but not limited to the above procedures.

A typical approach to synthesize the intermediate

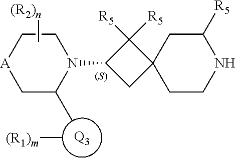

is described in Scheme 1-10 below.

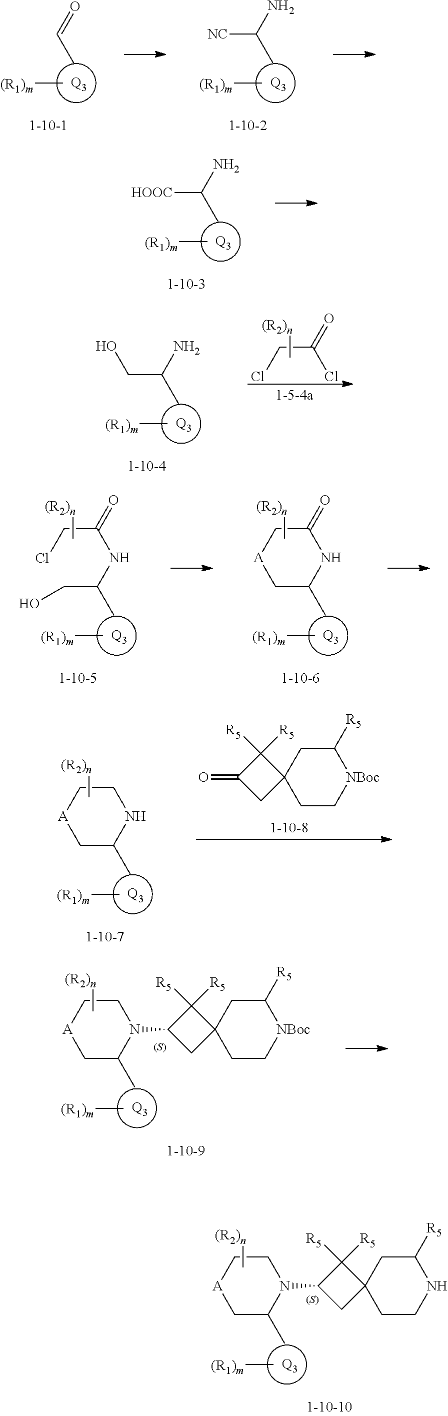

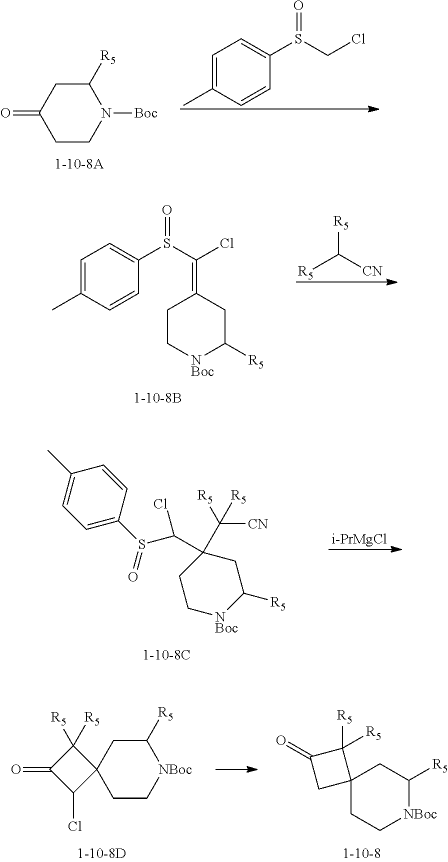

In Scheme 1-10, the starting material 1-10-1 can be prepared by conventional procedures using appropriate compounds and reagents. The starting material 1-10-1 can be converted to 1-10-3 through a two-step sequence of conventional reactions. After that, the carboxylic acid 1-10-3 can be reduced to 1-10-4 readily, which can react with 1-5-4a to give 1-10-5. Next, 1-10-5 can undergo an intramolecular cyclization to give 1-10-6, which is treated with a suitable reducing reagent to afford 1-10-7. And then 1-10-8 can be prepared by the method similar to Scheme 1-9 by using appropriate staring materials, and intermediates.

Finally, the reductive amination of 1-10-7 with 1-10-8 can generate 1-10-9, which is deprotected to give the target compounds 1-10-10.

Also, the target compounds can be synthesized by alternative methods but not limited to the above procedures.

A typical approach to synthesize the intermediate

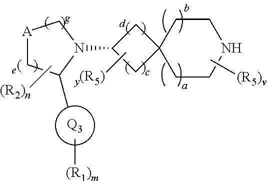

is described in Scheme 1-11 below.

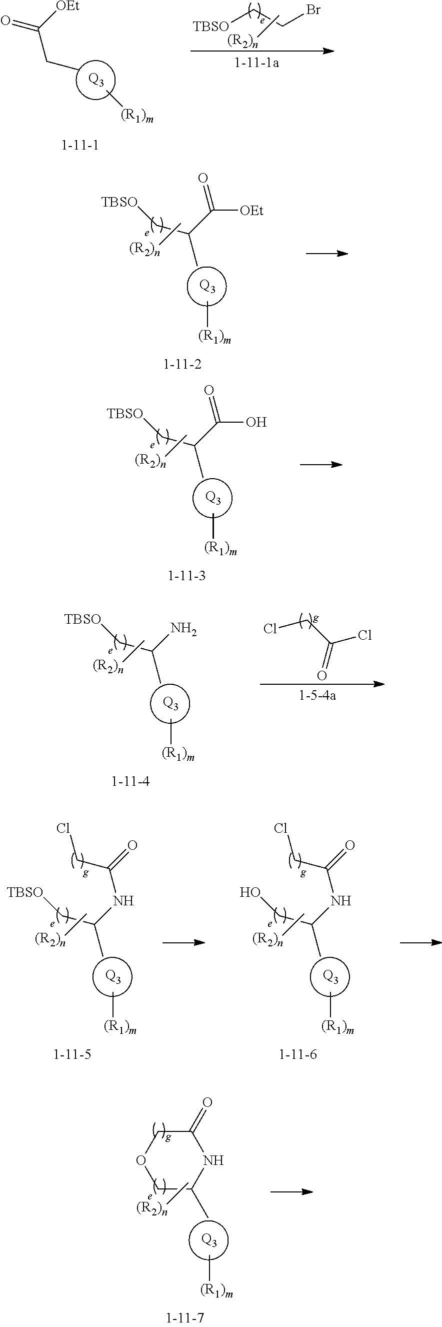

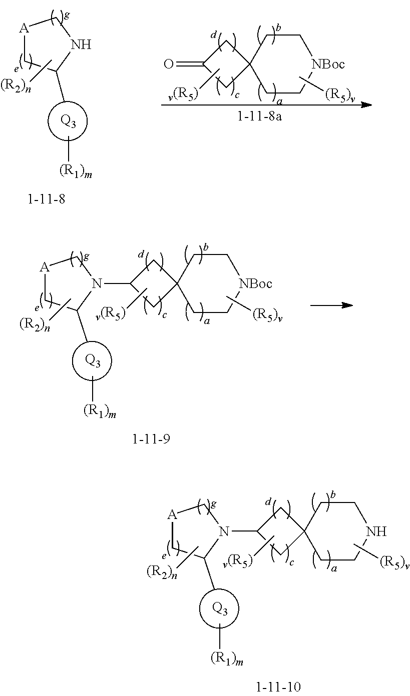

In Scheme 1-11, the starting material 1-11-1 can be prepared by conventional procedures using appropriate compounds and reagents. The starting material 1-11-1 can be converted to 1-11-3 through a two-step sequence of conventional reactions. After that, the carboxylic acid 1-11-3 can be converted to 1-11-4 readily, which can react with 1-5-4a to give 1-11-5. The deprotection of 1-11-5 can generate 1-11-6, which can be converted to 1-11-7 via an intramolecular cyclization reaction. The intermediate 1-11-7 can be reduced to afford 1-11-8, and then the reaction of 1-11-8 with 1-11-8a can give 1-11-9. Finally, the deprotection of 1-11-9 can generate the target compounds 1-11-10.

Also, the target compounds can be synthesized by alternative methods but not limited to the above procedures.

Similarly, the compounds of

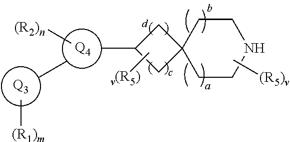

can be prepared by schemes similar to the Scheme 1-Scheme 11 by using appropriate staring materials and intermediates.

Similarly, the compounds of can be prepared by schemes similar to the Scheme A-Scheme E by using appropriate staring materials and intermediates.

Similarly, the compounds of

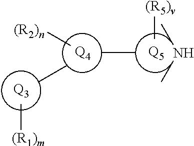

can be prepared by schemes similar to the Scheme 1-Scheme 11 by using appropriate staring materials and intermediates.

A typical approach to synthesize the intermediate

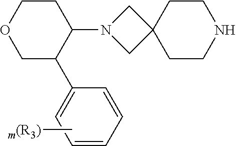

is described in Scheme 1-12 below.

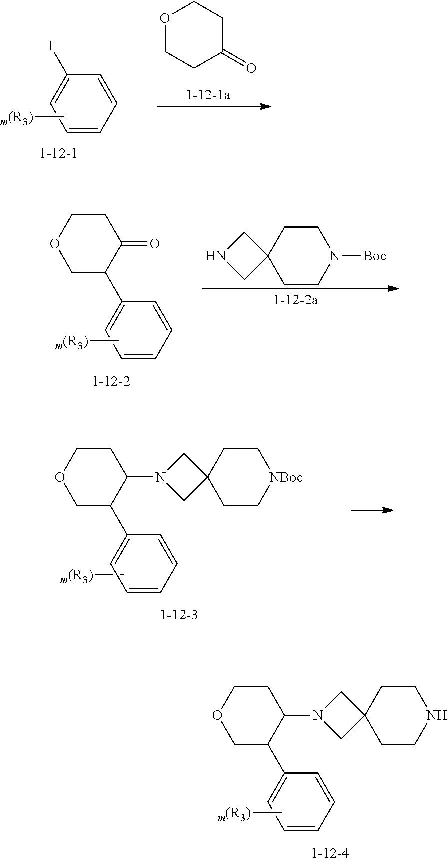

In Scheme 1-12, the starting material 1-12-1 can be prepared by conventional procedures using appropriate compounds and reagents. The starting material 1-12-1 can couple with 1-12-1a to give 1-12-2, which goes through a reductive amination reaction with 1-12-2a to yield the intermediate 1-12-3. Finally, the intermediate 1-12-3 is deprotected to generate the target compounds 1-12-4.

Also, the target compounds can be synthesized by alternative methods but not limited to the above procedures.

A typical approach to synthesize the intermediate

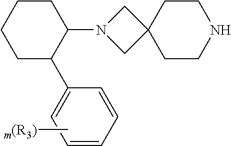

is described in Scheme 1-13 below.

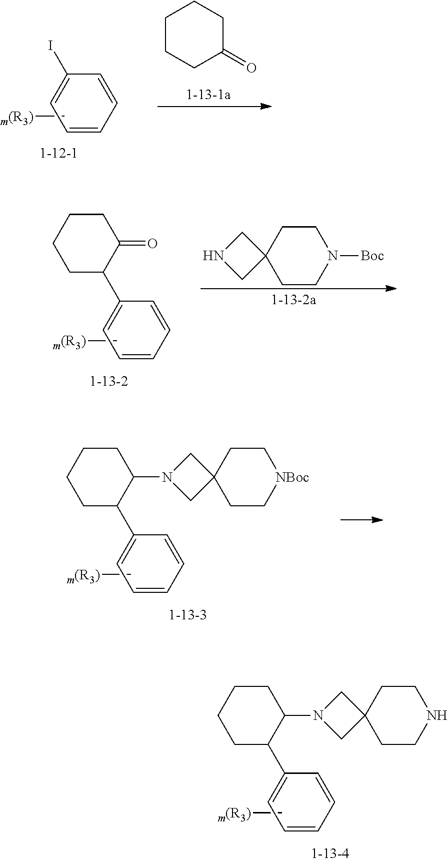

In Scheme 1-13, the starting material 1-12-1 can be prepared by conventional procedures using appropriate compounds and reagents. The starting material 1-12-1 can couple with 1-13-1a to give 1-13-2, which goes through a reductive amination reaction with 1.13-2a to yield the intermediate 1-13-3. Finally, the intermediate 1-13-3 is deprotected to generate the target compounds 1-13-4.

Also, the target compounds can be synthesized by alternative methods but not limited to the above procedures.

A typical approach to synthesize the intermediate

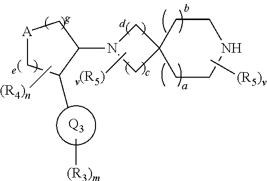

is described in Scheme 1-14 below.

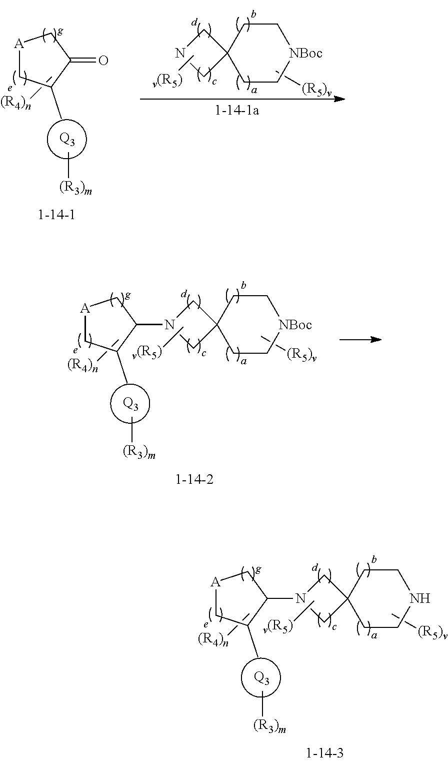

In Scheme 1-14, the starting material 1-14-1 can be prepared by conventional procedures using appropriate compounds and reagents. The starting material 1-14-1 goes through a reductive amination reaction with 1-14-1a to yield the intermediate 1-14-2, which is deprotected to generate the target compounds 1-14-3.

Also, the target compounds can be synthesized by alternative methods but not limited to the above procedures.

A typical approach to synthesize the intermediate

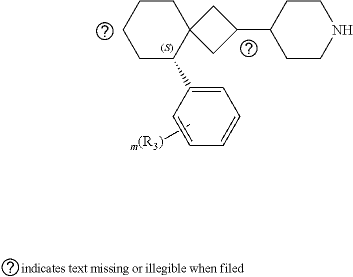

is described in Scheme 1-15 below.

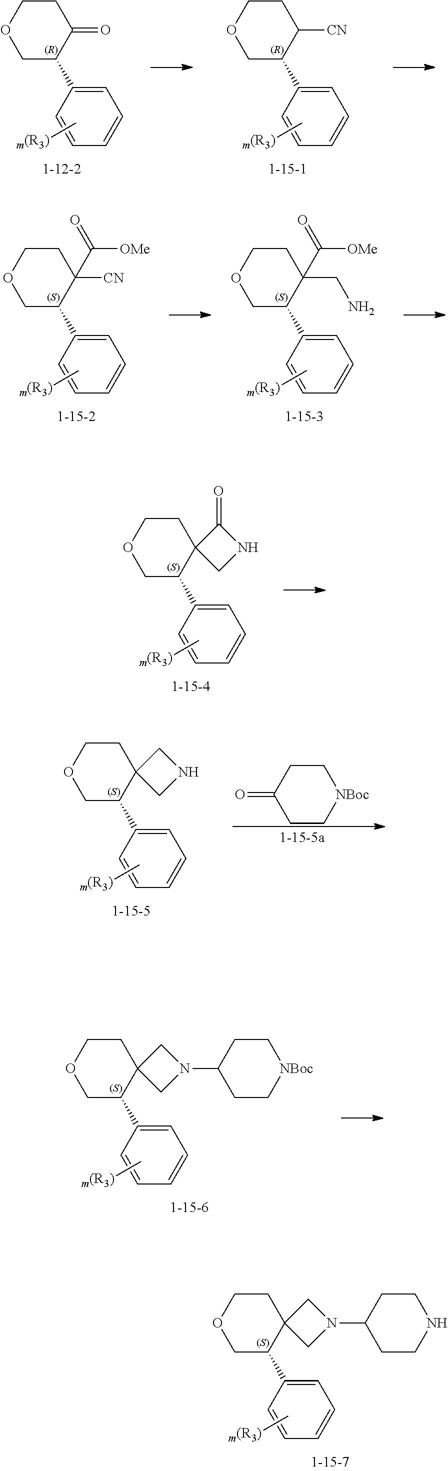

In Scheme 1-10, the starting material 1-12-2 can be prepare by conventional procedures using appropriate compounds and reagents. The starting material 1-12-2 can be converted to 1-15-1 readily, which is converted to 1-15-2 through a conventional organic reaction. After that, the intermediate 1-15-2 is reduced to give 1-15-3, which can be converted to 1-15-4 though an intramolecular cyclization reaction. Next, 1-15-4 is reduced to yield 1-15-5, which can react with 1-15-5a to afford the intermediate 1-15-6. Finally, the intermediate 1-15-6 is deprotected to generate the target compounds 1-15-7.

Also, the target compounds can be synthesized by alternative methods but not limited to the above procedures.

A typical approach to synthesize the intermediate

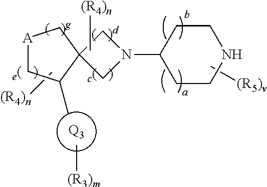

is described in Scheme 1-16 below.

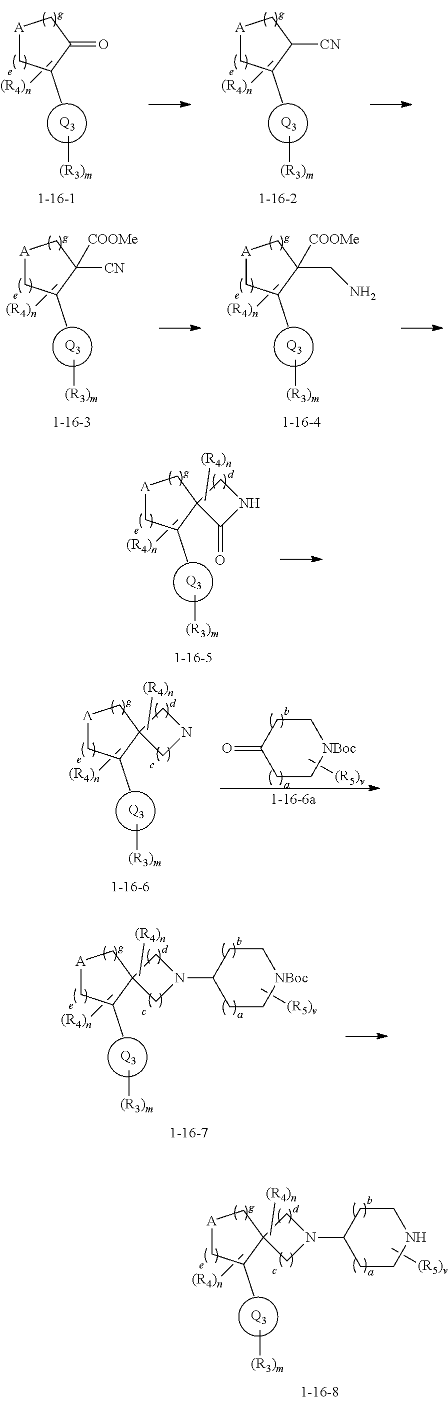

In Scheme 1-16, the starting material 1-16-1 can be prepare by conventional procedures using appropriate compounds and reagents. The starting material 1-16-1 can be converted to 1-16-2 readily, which is converted to 1-16-3 through a conventional organic reaction. After that, the intermediate 1-16-3 is reduced to give 1-16-4, which can be converted to 1-16-5 though an intramolecular cyclization. Next, 1-16-5 is reduced to yield 1-16-6, which can react with 1-16-6a to afford the intermediate 1-16-7. Finally, the intermediate 1-16-7 is deprotected to generate the target compounds 1-16-8.

Also, the target compounds can be synthesized by alternative methods but not limited to the above procedures.

A typical approach to synthesize the intermediate

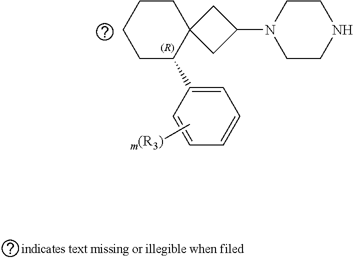

is described in Scheme 1-17 below.

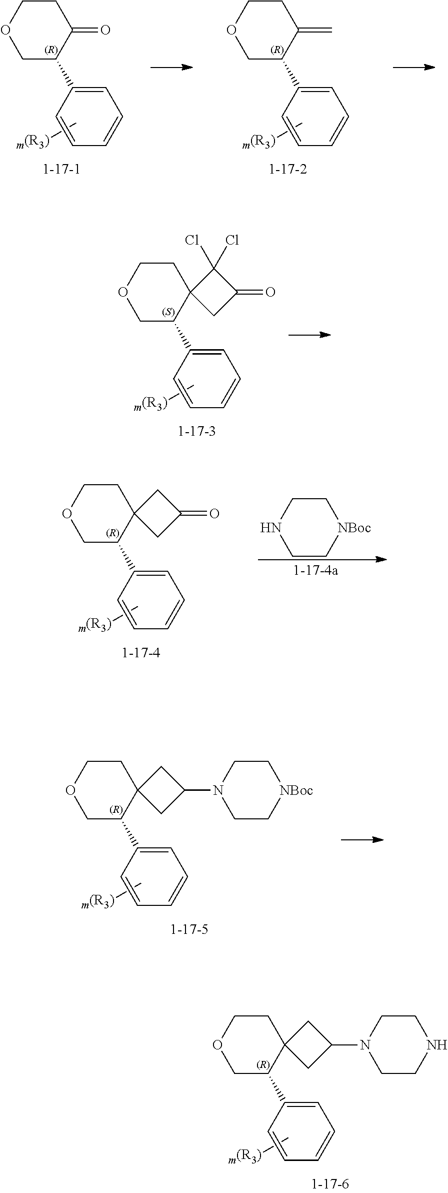

In Scheme 1-17, the starting material 1-17-1 can be prepared by conventional procedures using appropriate compounds and reagents. The starting material 1-17-1 can be converted to 1-17-2 via a general Wittig reaction, which further undergoes Staudinger ketene cycloaddition to give 1-17-3. After that, the dichloride 1-17-3 can be reduced to 1-17-4 by a suitable reagent, and then the reaction of 1-17-4 with 1-17-4a can generate 1-17-5. Finally, the deprotection of 1-17-5 can give the target compounds 1-17-6.

Also, the target compounds can be synthesized by alternative methods but not limited to the above procedures.

A typical approach to synthesize the intermediate

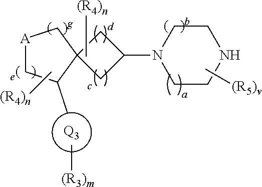

is described in Scheme 1-18 below.

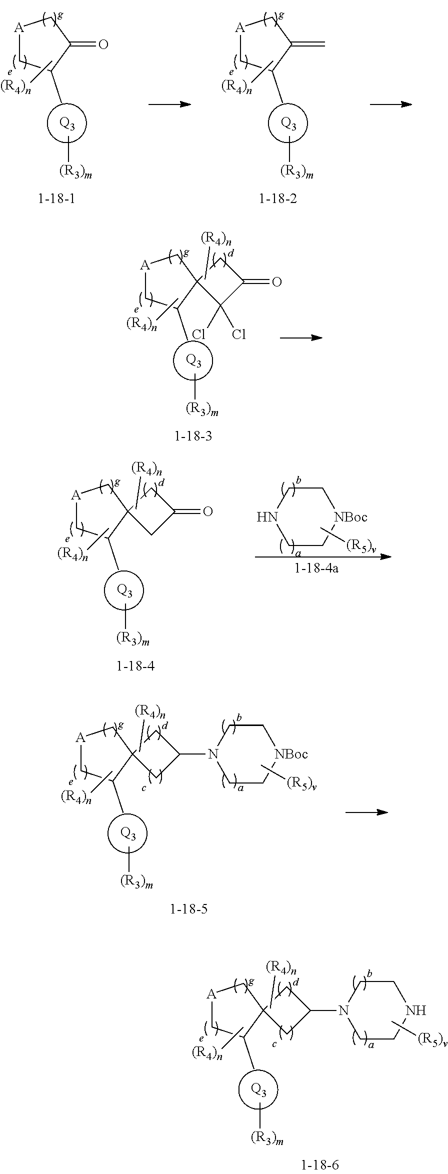

In Scheme 1-18, the starting material 1-18-1 can be prepared by conventional procedures using appropriate compounds and reagents. The starting material 1-18-1 can be converted to 1-18-2 via a general Wittig reaction, which further undergoes Staudinger ketene cycloaddition to give 1-18-3. After that, the dichloride 1-18-3 can be reduced to 1-18-4 by a suitable reagent, and then the reaction of 1-18-4 with 1-18-4a can generate 1-18-5. Finally, the deprotection of 1-18-5 can give the target compounds 1-18-6.

Also, the target compounds can be synthesized by alternative methods but not limited to the above procedures.

A typical approach to synthesize the intermediate

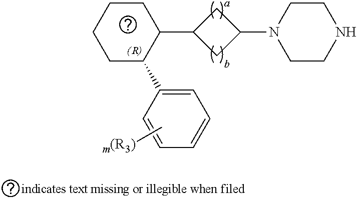

is described in Scheme 1-19 below.

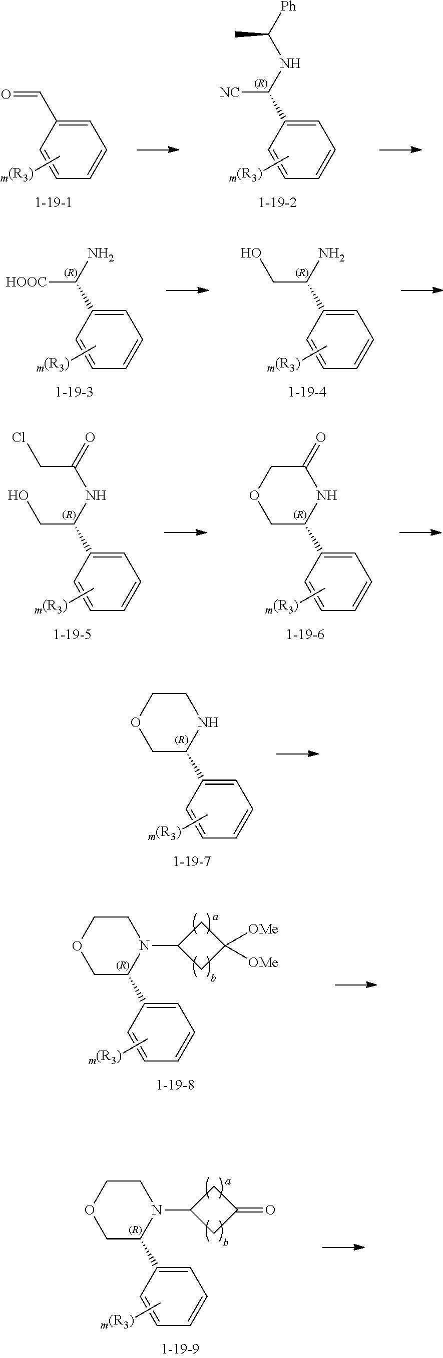

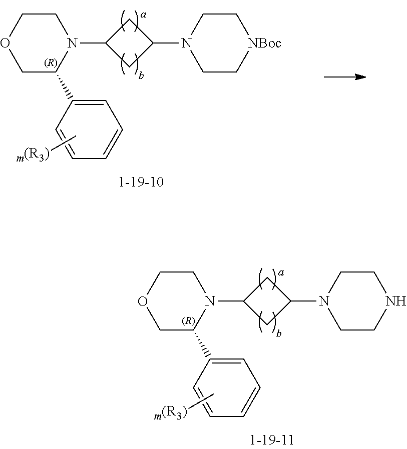

In Scheme 1-19, the starting material 1-19-1 can be prepared by conventional procedures using appropriate compounds and reagents. The starting material 1-19-1 can be converted to 1-19-2 via a Strecker reaction, which can be converted to 1-19-3 readily. After that, the intermediate 1-19-3 is reduced to 1-19-4 by a suitable reagent. The intermediate 1-19-4 can be converted to 1-19-5, which undergoes an intramolecular cyclization to give 1-19-6. Next, the intermediate 1-19-6 is reduced to give 1-19-7, which can be converted to 1-19-9 through a two-step sequence conventional reaction. Finally, the intermediate 1-19-9 can be converted to 1-19-10, which is deprotected to yield the target compounds 1-19-11.

Also, the target compounds can be synthesized by alternative methods but not limited to the above procedures.

A typical approach to synthesize the intermediate

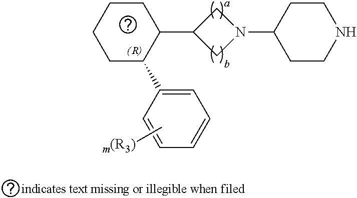

is described in Scheme 1-20 below.

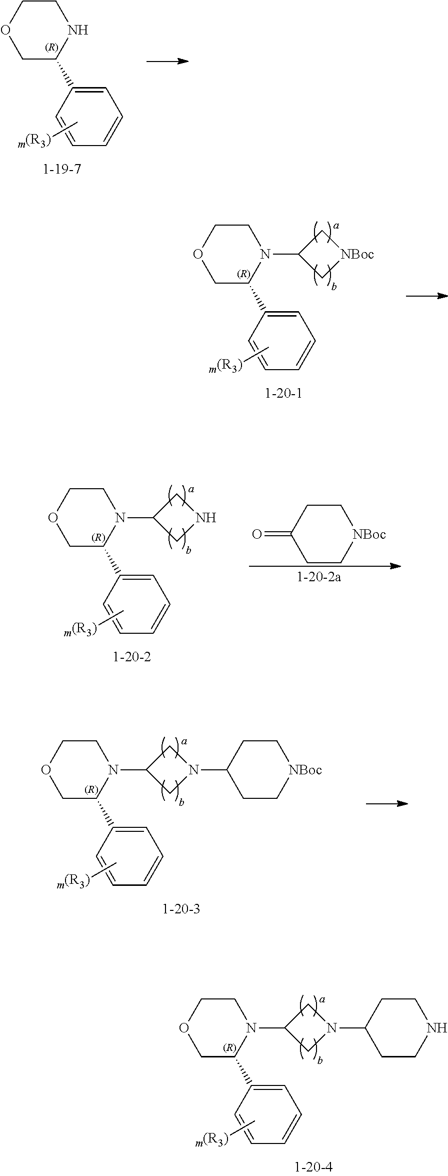

In Scheme 1-20, the starting material 1-19-7 can be prepared by conventional procedures using appropriate compounds and reagents. The starting material 1-19-7 can be converted to 1-20-1 via a reductive amination reaction, which is deprotected to give 1-20-2. Finally, the intermediate 1-20-2 can react with 1-20-2a to afford 1-20-3, which is deprotected to yield the target compounds 1-20-4.

Also, the target compounds can be synthesized by alternative methods but not limited to the above procedures.

Similarly, the compounds of

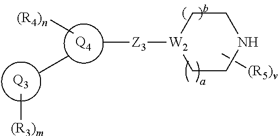

in which W₂ is N or C(Rₐ) can be prepared by schemes similar to the Scheme 1-19 to Scheme 1-20 by using appropriate staring materials and intermediates.

A typical approach to synthesize the intermediate

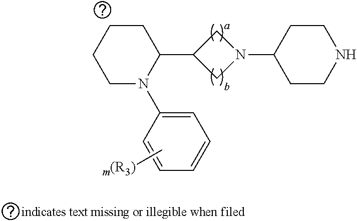

is described in Scheme 1-21 below.

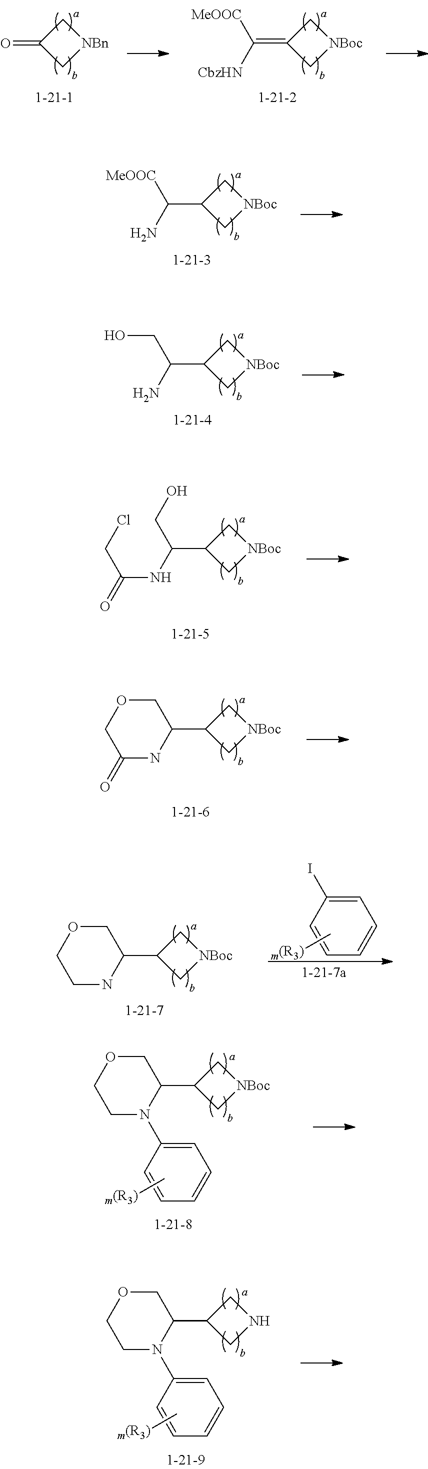

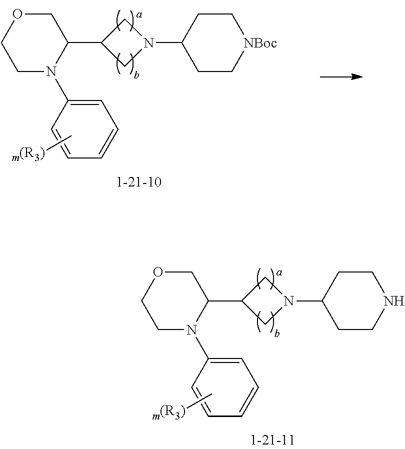

In Scheme 1-21, the starting material 1-21-1 can be prepared by conventional procedures using appropriate compounds and reagents. The starting material 1-21-1 can be converted to 1-21-2 readily, which can be reduced to 1-21-3. After that, the intermediate 1-21-3 is further reduced to 1-21-4 by a suitable reagent. The intermediate 1-21-4 can be converted to 1-21-5, which undergoes an intramolecular cyclization to give 1-21-6. Next, the intermediate 1-21-6 is reduced to give 1-21-7, which can couple with 1-21-7a to give 1-21-8. The intermediate 1-21-8 is deprotected to give 1-21-9. Finally, the intermediate 1-21-9 can be converted to 1-21-10, which is deprotected to yield the target compounds 1-21-11.

Also, the target compounds can be synthesized by alternative methods but not limited to the above procedures.

A typical approach to synthesize the intermediate

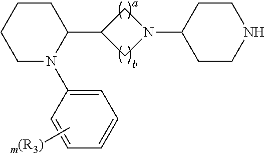

is described in Scheme 1-22 below.

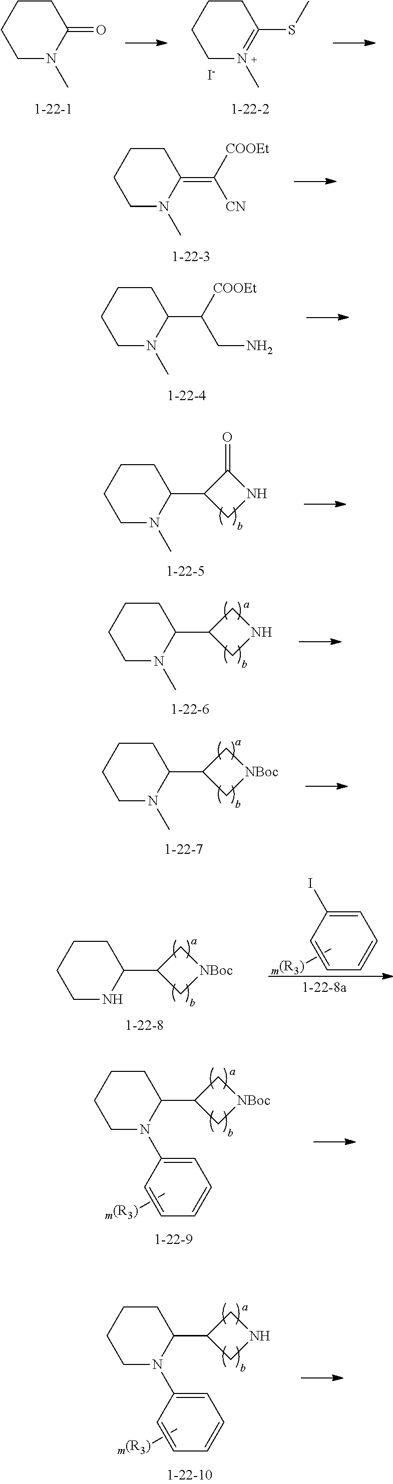

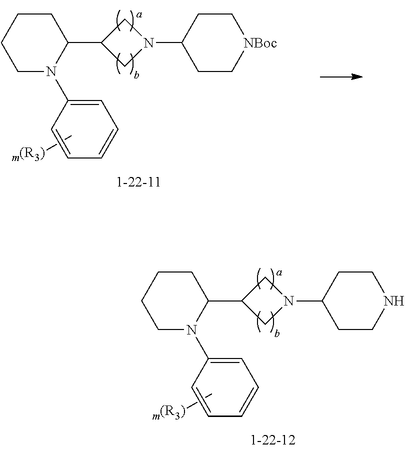

In Scheme 1-22, the starting material 1-22-1 can be prepared by conventional procedures using appropriate compounds and reagents. The starting material 1-22-1 can be converted to 1-22-2 readily, which is converted to 1-22-3 by a literate known condition. After that, the intermediate 1-22-3 is reduced to give 1-22-4 by a suitable reagent. The intermediate 1-22-4 can be converted to 1-22-5 through an intramolecular cyclization, which is reduced to give 1-22-6. Next, the intermediate 1-22-6 is protected to give 1-22-7, and then the de-methylation of 1-22-7 can give the intermediate 1-22-8. The intermediate 1-22-8 can couple with 1-22-8a to afford 1-22-9, which is deprotected to give 1-22-10. Finally, the intermediate 1-22-10 can be converted to 1-22-11, which is deprotected to yield the target compounds 1-22-12.

Also, the target compounds can be synthesized by alternative methods but not limited to the above procedures.

Similarly, the compounds of

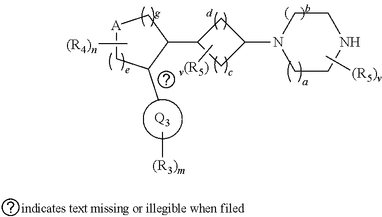

can be prepared by schemes similar to the Scheme 1-21 to Scheme 1-22 by using appropriate staring materials and intermediates.

An approach to synthesize of target compounds of

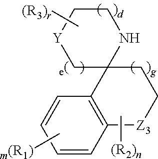

is described in Scheme 1-23:

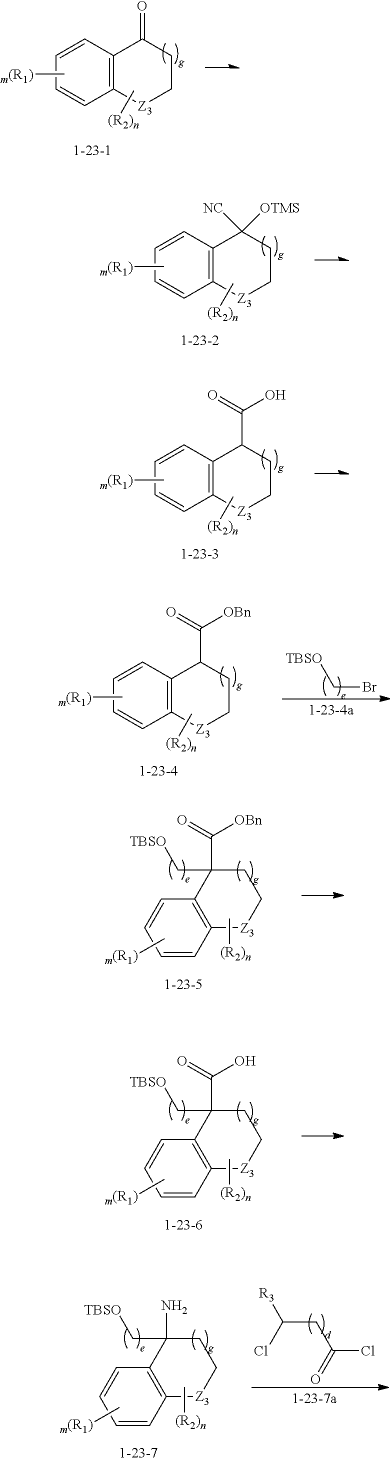

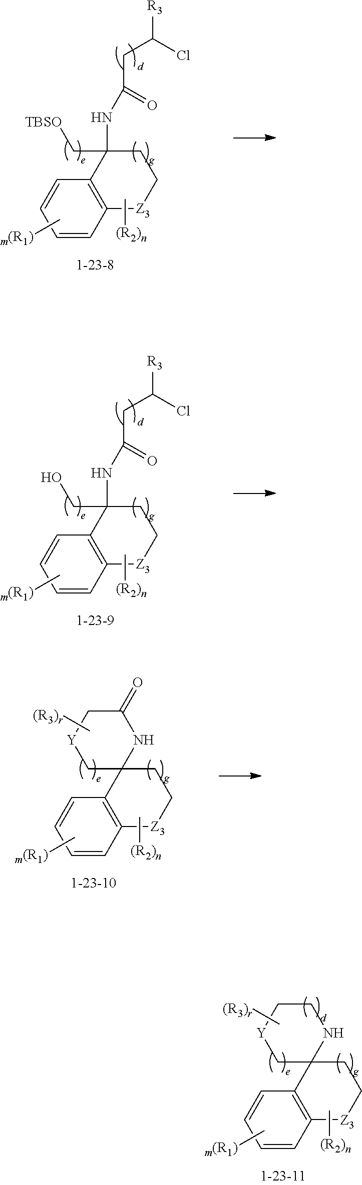

In Scheme 1-23, the starting material 1-23-1 can be prepared by conventional procedures using appropriate compounds and reagents. The starting material 1-23-1 can be converted to 1-23-3 through a two-step sequence of conventional organic reactions. After that, the carboxylic acid 1-23-3 can be converted to 1-23-4 readily, which can react with 1-23-4a to give intermediate 1-23-5. The deprotection of 1-23-5 can generate 1-23-6, which can be converted to 1-23-7 via a Curtius rearrangement reaction. The intermediate 1-23-7 can react with 1-23-7a to afford 1-23-8, and then deprotected of 1-23-8 can give the intermediate 1-23-9. Finally, 1-23-9 can undergo an intramolecular cyclization to afford the intermediate 1-23-10, which is treated with a suitable reductive reagent to afford the target compounds 1-23-11.

Also, the target compounds can be synthesized by alternative methods but not limited to the above procedures.

A typical approach to synthesize the intermediate

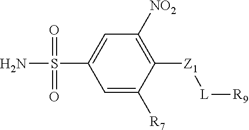

is described in Scheme 2.

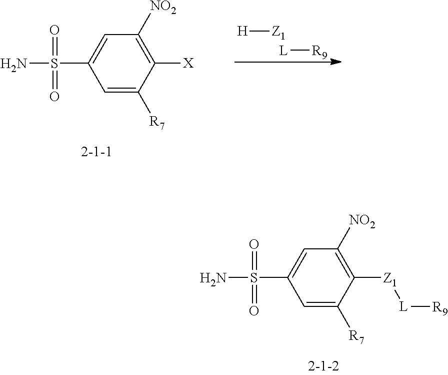

In Scheme 2-1, the starting material 2-1-1 reacts with appropriate alcohol or amine will yield 2-1-2.

Also, the target compounds can be synthesized by alternative methods but not limited to the above procedures.

Similarly, the intermediate o

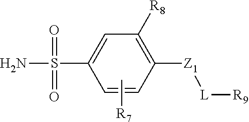

can be prepared by the method similar to Scheme 2-1 by using appropriate staring materials, and intermediates.

A typical approach to synthesize the intermediate in which is described in Scheme 2-2 below.

A typical approach to synthesize the intermediate

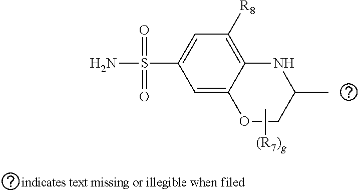

is described in Scheme 2.2 below.

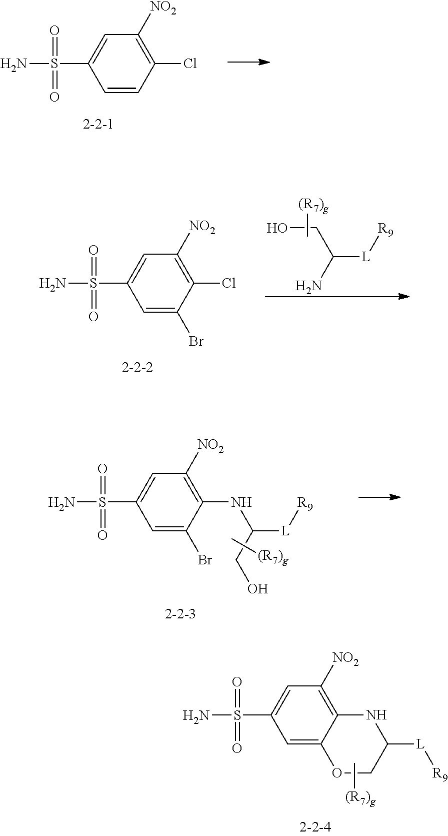

In Scheme 2-2, the bromination of the commercially available 2-2-1 results in 2-2-2, and then the reaction of 2-2-2 with appropriate amine provides 2-2-3. Intramolecular cyclization of 2-2-3 using metal-catalyzed coupling condition such as Buchwald reaction or other coupling reaction known in the literatures give 2-2-4. Alternatively, 2-2-4 can be obtained via a three-step sequence of mesylation of the hydroxyl group of 2-2-3, SN₂ reaction and intramolecular cyclization.

Also, the target compounds can be synthesized by alternative methods but not limited to the above procedures.

A typical approach to synthesize the intermediate

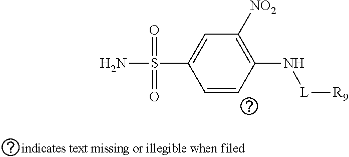

is described in Scheme 2-3 below.

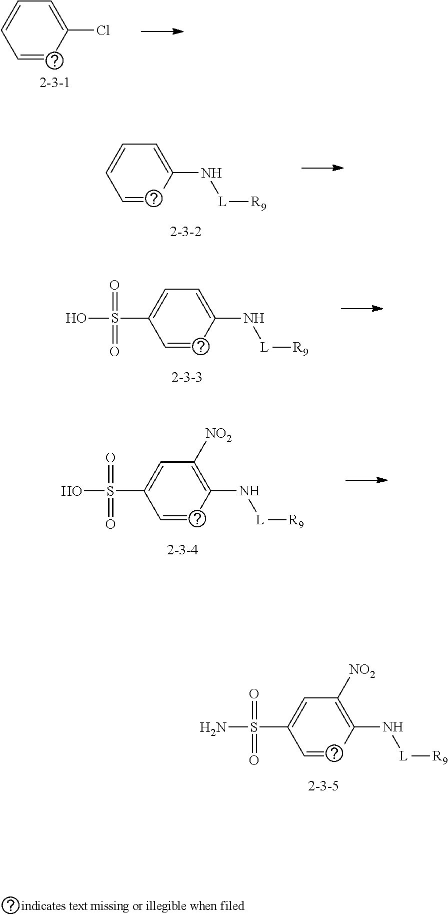

In Scheme 2-3, the starting material 2-3-1 can be prepared by conventional procedures using appropriate compounds and reagents. The starting material 2-3-1 can be converted to 2-3-2 via a SNAr reaction, and then the sulfonylation of 2-3-2 can afford the intermediate 2-3-3. Finally, the intermediate 2-3-3 goes through a two-step sequence of conventional organic reaction to yield the target compounds 2-3-5.

Also, the target compounds can be synthesized by alternative methods but not limited to the above procedures.

A typical approach to synthesize the intermediate

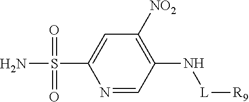

is described in Scheme 2-4 below.

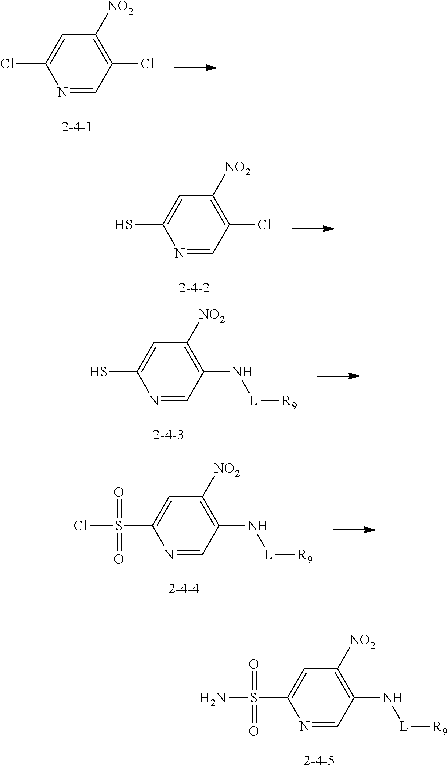

In Scheme 2-4, the starting material 2-4-1 can be prepared by conventional procedures using appropriate compounds and reagents. The starting material 2-4-1 can be converted to 2-4-3 via a two-step sequence of conventional organic reaction. Finally, the intermediate 2-4-3 can be converted to the corresponding sulfonyl chlorides, which can react with ammonia to yield the target compounds 2-4-5.

Also, the target compounds can be synthesized by alternative methods but not limited to the above procedures.

A typical approach to synthesize the intermediate

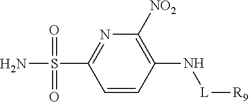

is described in Scheme 2-5

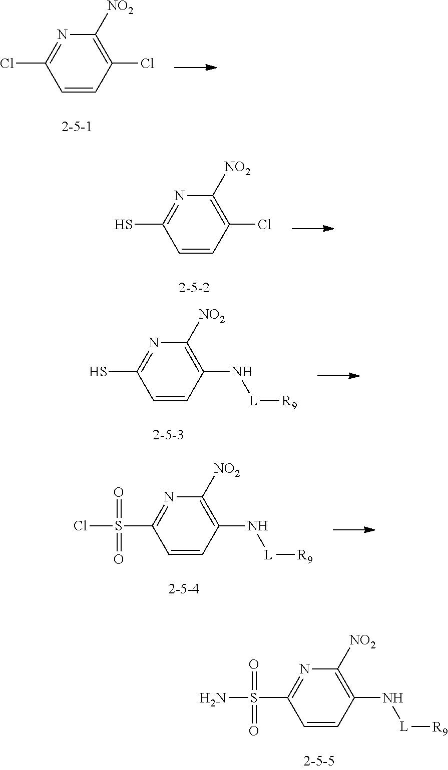

In Scheme 2-5, the starting material 2-5-1 can be prepared by conventional procedures using appropriate compounds and reagents. The starting material 2-5-1 can be converted to 2-5-3 via a two-step sequence conventional organic reaction. Finally, the intermediate 2-5-3 can be converted to the corresponding sulfonyl chlorides 2-5-4, which can react with ammonia to yield the target compounds 2-5-5.

Also, the target compounds can be synthesized by alternative methods but not limited to the above procedures.

A typical approach to synthesize the intermediate

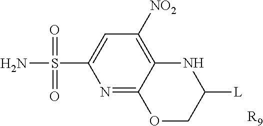

is described in Scheme 2-6 below.

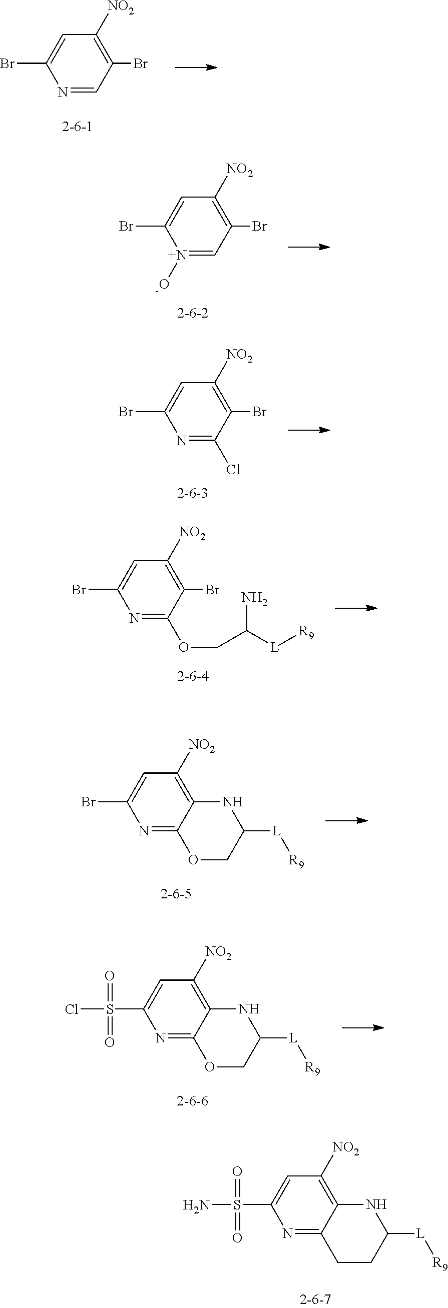

In Scheme 2-6, the starting material 2-6-1 can be prepared by conventional procedures using appropriate compounds and reagents. The starting material 2-6-1 can be oxidized to yield 2-6-2, which can be converted to the intermediate 2-6-3 readily. After that, the intermediate 2-6-3 undergoes a SNAr reaction to give 2-6-4, which is converted to 2-6-5 through an intramolecular cyclization reaction. Finally, the intermediate 2-6-5 can be converted to the corresponding sulfonyl chlorides 2-6-6, which can react with ammonia to yield the target compounds 2-6-7.

Also, the target compounds can be synthesized by alternative methods but not limited to the above procedures.

A typical approach to synthesize the intermediate

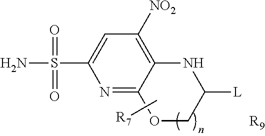

in which h is 0, 1, or 2 is described in Scheme 2-7 below.

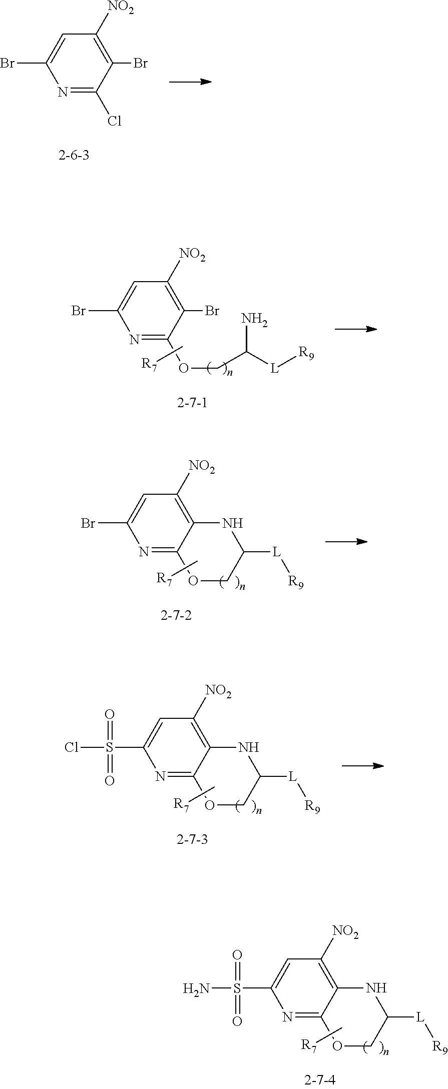

In Scheme 2-7, the starting material 2-6-3 can be prepared by conventional procedures using appropriate compounds and reagents. The starting material 2-6-3 undergoes a SNAr reaction to give 2-7-1, which is converted to 2-7-2 through an intramolecular cyclization reaction. Finally, the intermediate 2-7-2 can be converted to the corresponding sulfonyl chlorides 2-7-3, which can further react with ammonia to yield the target compounds 2-7-4.

Also, the target compounds can be synthesized by alternative methods but not limited to the above procedures.

A typical approach to synthesize the intermediate

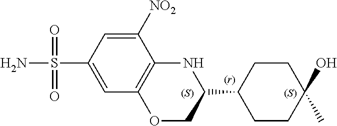

is described in Scheme 2-8 below.

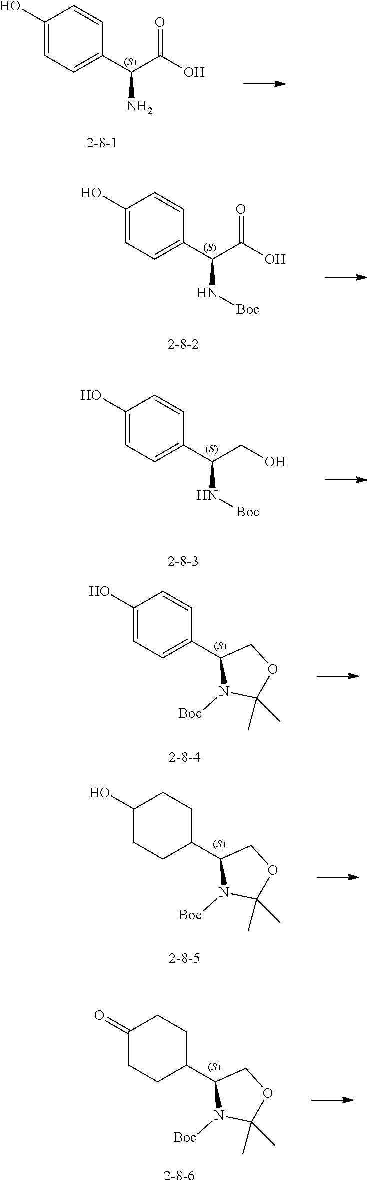

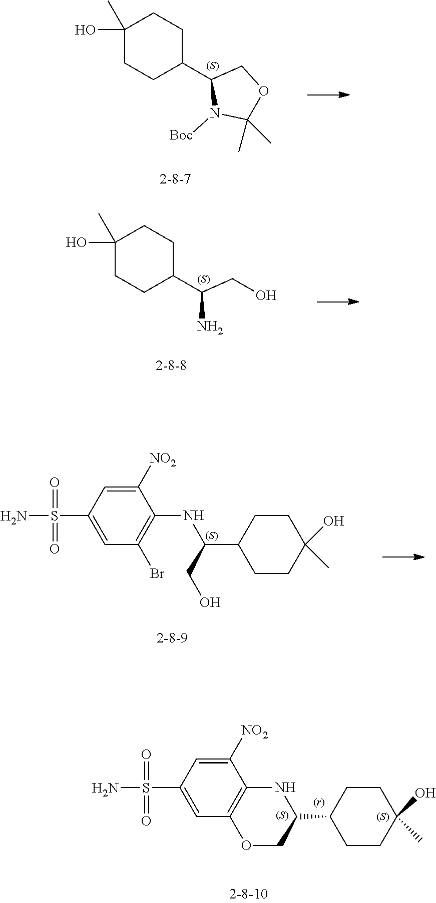

In Scheme 2-8, the starting material 2-8-1 can be prepared by conventional procedures using appropriate compounds and reagents. The starting material 2-8-1 can be converted to 2-8-3 through a two-step sequence of conventional organic reactions. After that, the intermediate 2-8-3 is protected to give 2-8-4, which can be further reduced to yield the intermediate 2-8-5. The intermediate 2-8-5 is oxidized to generate the ketone 2-8-6, which can be converted to 2-8-7 readily. The intermediate 2-8-7 is deprotected to afford 2-8-8, which can be further converted to the intermediate 2-8-9. Finally, 2-8-9 undergoes an intramolecular cyclization to afford the target compounds 2-8-10.

Also, the target compounds can be synthesized by alternative methods but not limited to the above procedures.

A typical approach to synthesize the intermediate

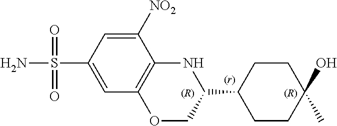

is described in Scheme 2-9 below.

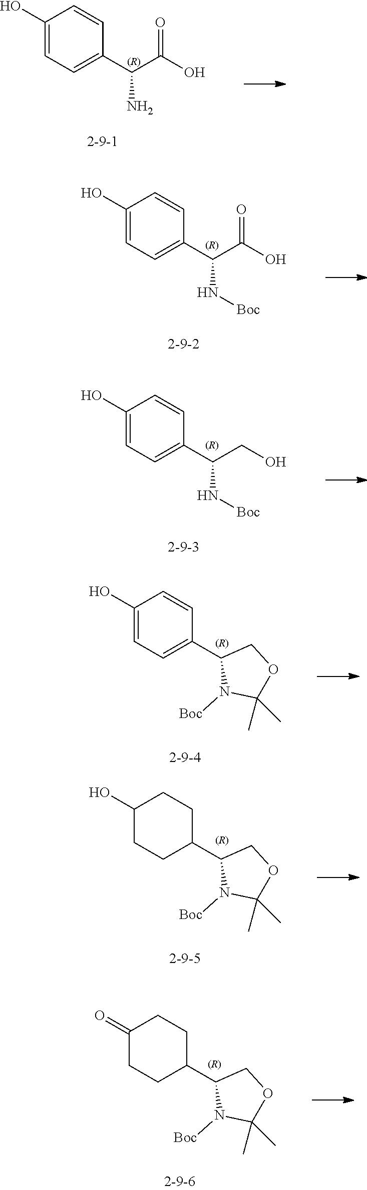

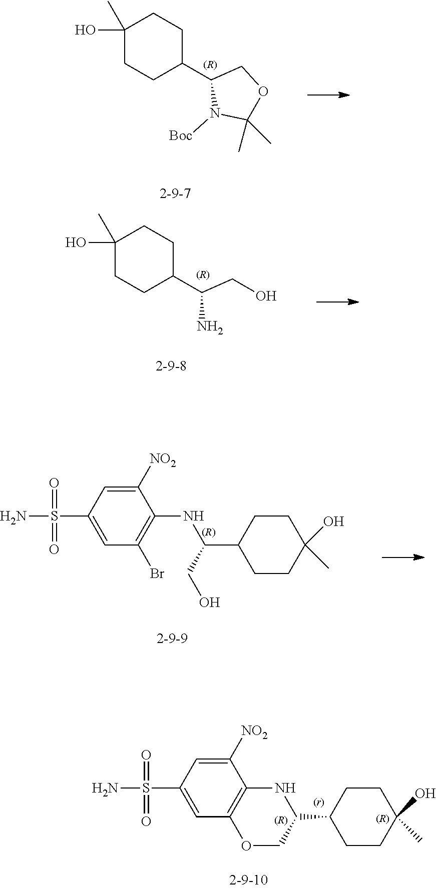

In Scheme 2-9, the starting material 2-9-1 can be prepared by conventional procedures using appropriate compounds and reagents. The starting material 2-9-1 can be converted to 2-9-3 through a two-step sequence of conventional organic reactions. After that, the intermediate 2-9-3 is protected to give 2-9-4, which can be further reduced to yield the intermediate 2-9-5. The intermediate 2-9-5 is oxidized to generate the ketone 2-9-6, which can be converted to 2-9-7 readily. The intermediate 2-9-7 is deprotected to afford 2-9-8, which can be further converted to the intermediate 2-9-9. Finally, 2-9-9 undergoes an intramolecular cyclization to afford the target compounds 2-9-10.

Also, the target compounds can be synthesized by alternative methods but not limited to the above procedures.

A typical approach to synthesize the intermediate

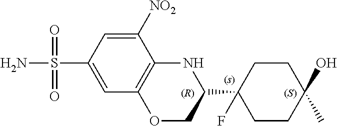

is described in Scheme 2-10 below.

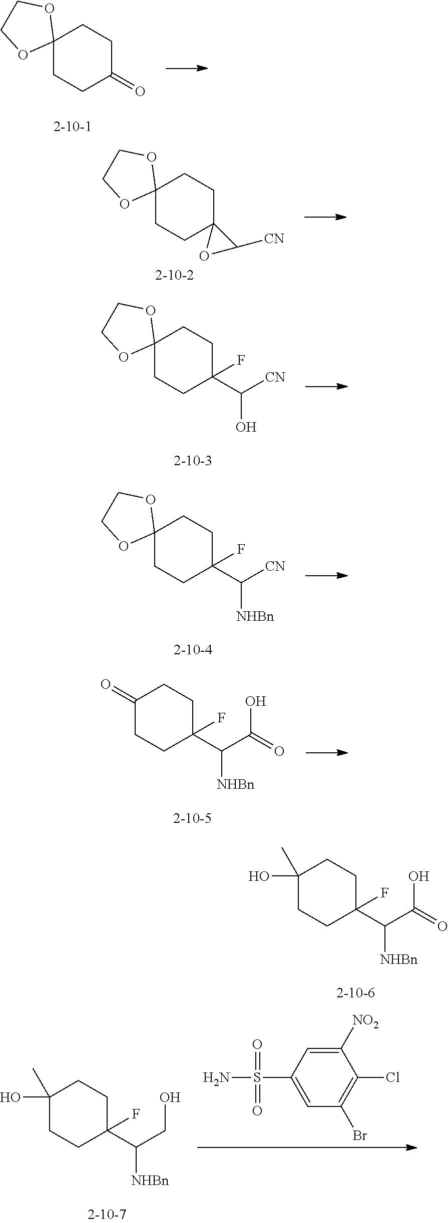

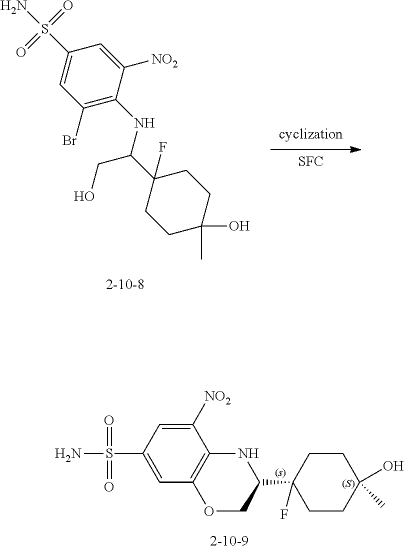

In Scheme 2-10, the starting material 2-10-1 can be prepared by conventional procedures using appropriate compounds and reagents. The starting material 2-10-1 can be converted to 2-10-3 through a two-step sequence of literate reported reactions. After that, the intermediate 2-10-3 can be converted to 2-10-4, which can be further hydrolyzed to yield the intermediate 2-10-5. The intermediate 2-10-5 can be converted to 2-10-6 readily, which is reduced to give 2-10-7. Finally, the intermediate 2-10-7 can be converted to the intermediate 2-10-8, which undergoes an intramolecular cyclization and through a further chiral separation to afford the target compounds 2-10-9.

Also, the target compounds can be synthesized by alternative methods but not limited to the above procedures.

A typical approach to synthesize the intermediate

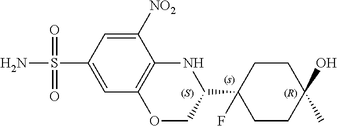

is described in

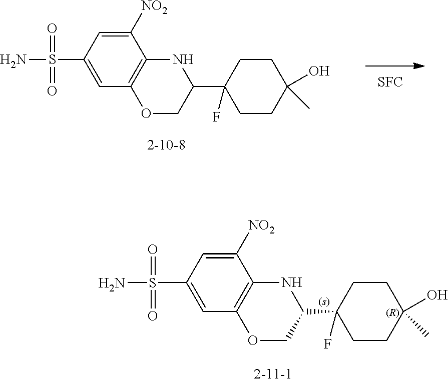

In Scheme 2-11, the starting material 2-10-8 can be prepared by conventional procedures using appropriate compounds and reagents. The starting material 2-10-8 can goes through a chiral separation to afford the target compound 2-11-1.

Also, the target compounds can be synthesized by alternative methods but not limited to the above procedures.

Similarly, the intermediate of

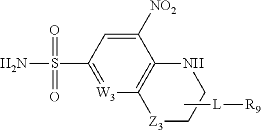

in which with is C(Rₐ) or N; and different Z₃ and can be prepared by the method similar to the Scheme 2-2 to Scheme 2-6 by using appropriate staring materials, intermediates, and intramolecular cylization.

Similarly, the intermediate of

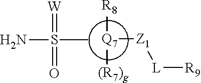

with different R₇ and Q₇ can be prepared by the method similar to the Scheme 2-1 and 2.2 by using appropriate staring materials, and intermediates.

A typical approach to synthesize the intermediate

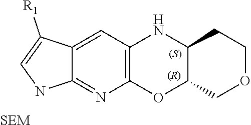

is described in Scheme 3-0-A below.

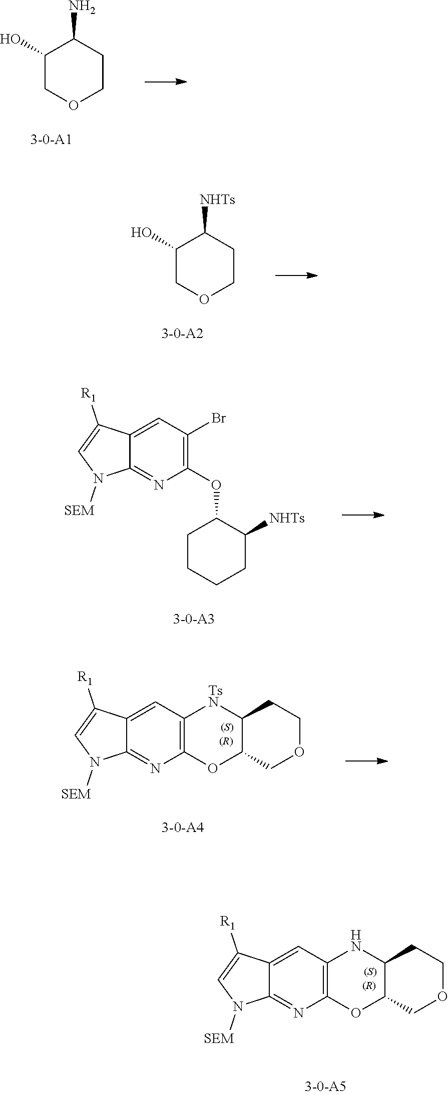

In Scheme 3-0-A, the starting material 3-0-A1 can be prepared by conventional procedures using appropriate compounds and reagents. The starting material 3-0-A1 can be converted to 3-0-A2 readily, which further undergoes a SNAr reaction to give 3-0-A3. Finally, 3-0-A3 can be converted to 3-0-A4 through an intramolecular cyclization reaction, which is deprotected to afford the target compounds 3-0-A5.

Also, the target compounds can be synthesized by alternative methods but not limited to the above procedures.

A typical approach to synthesize the intermediate

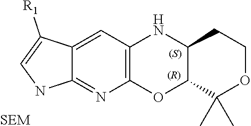

is described in Scheme 3-0-B below.

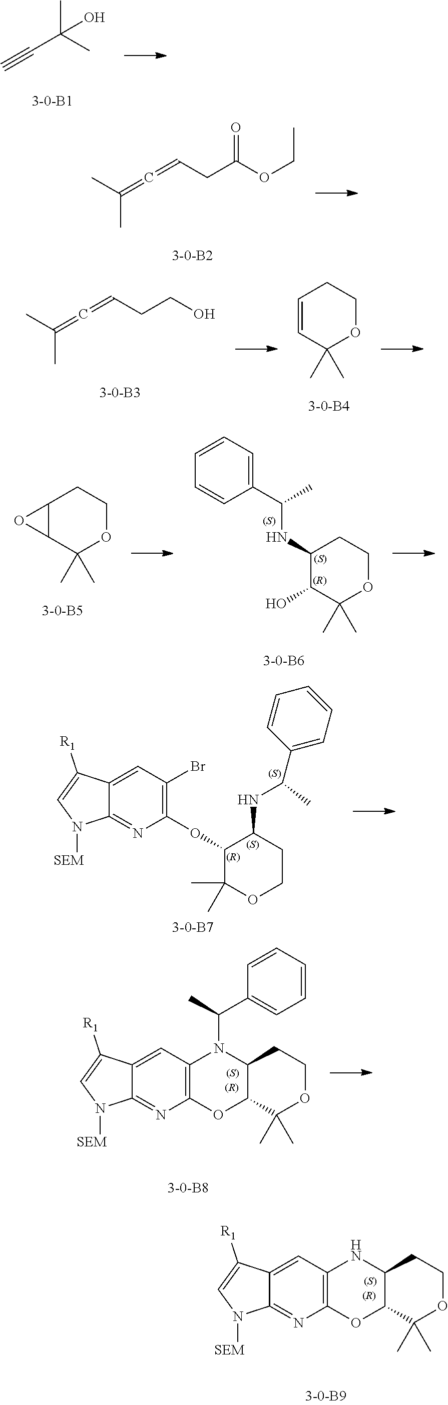

In Scheme 3-0-B, the starting material 3-0-B1 can be prepared by conventional procedures using appropriate compounds and reagents. The starting material 3-0-B1 can be converted to 3-0-B3 via a literature known reactions, which further undergoes a ring closing reaction to give 3-0-B4. After that, 3-0-B4 can be oxidied to 3-0-B5 readily, which is further converted to the intermediate 3-0-B6. Next, the intermediate 3-0-B6 undergoes a SNAr reaction to yield 3-0-B7, which can further go through an intramolecular coupling reaction to give 3-0-B8. Finally, the intermediate 3-0-B8 can be deprotected to afford the target compounds 3-0-B9.

Also, the target compounds can be synthesized by alternative methods but not limited to the above procedures.

A typical approach to synthesize the intermediate

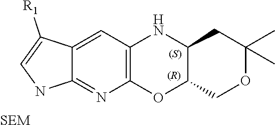

is described in Scheme 3. 0-C below.

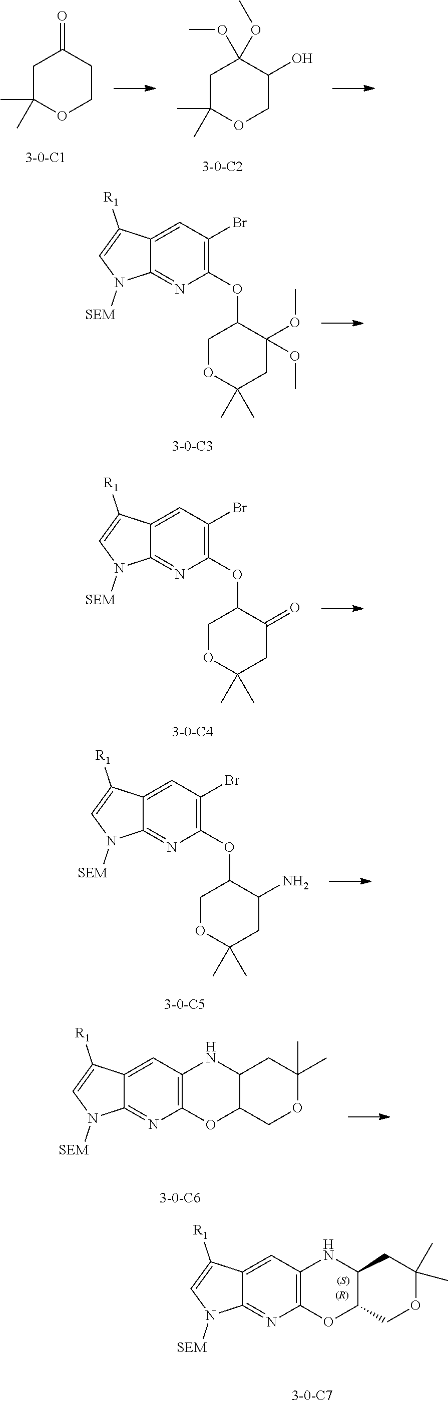

In Scheme 3-0-C, the starting material 3-0-C1 can be prepared by conventional procedures using appropriate compounds and reagents. The starting material 3-0-C1 can be converted to 3-0-C2 via a literature known reactions, which undergoes a SNAr reaction to yield 3-0-C3. After that, the intermediate 3-0-C3 is deprotected to give 3-0-C4, which can be converted to the intermediate 3-0-C5 via a reductive amination reaction. Finally, the intermediate 3-0-C5 goes through an intramolecular coupling reaction to afford the trans-racemate compounds 3-0-C6, which further go through a chiral separation to yield the target compounds 3-0-C7.

Also, the target compounds can be synthesized by alternative methods but not limited to the above procedures.

A typical approach to synthesize the intermediate

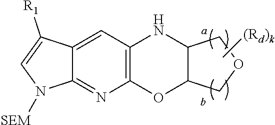

is described in Scheme 3.1 below.

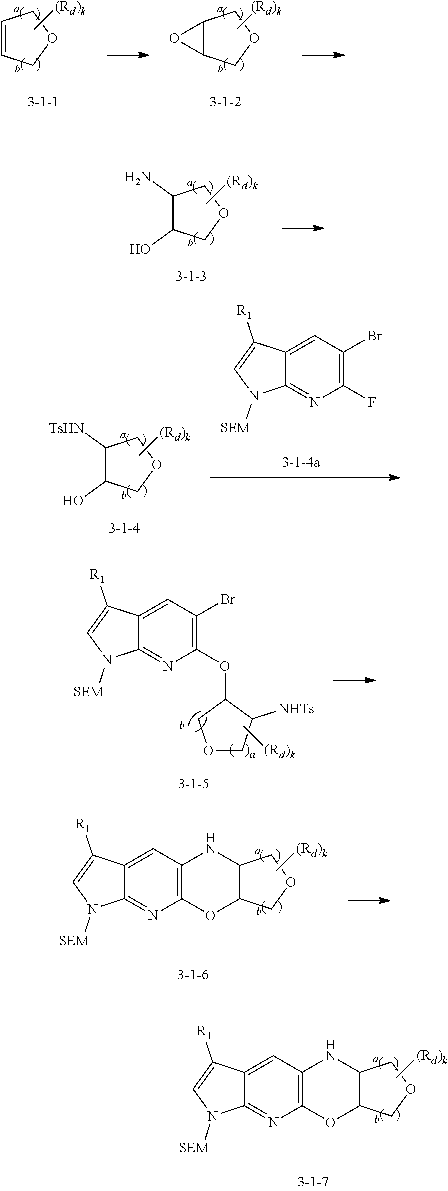

In Scheme 3-1, the starting material 3-1-1 can be prepared by conventional procedures using appropriate compounds and reagents. The starting material 3-1-1 can be converted to 3-1-2 readily, which further undergoes a ring opening reaction to give 3-1-3. After that, 3-1-3 can be converted to 3-1-4 via a general condition, and the reaction of 3-1-3 with 3-1-4a can generate intermediate 3-1-5. The intermediate 3-1-5 undergoes an intramolecular coupling reaction to give 3-1-6, which is further deprotected to afford 3-1-7.

Also, the target compounds can be synthesized by alternative methods but not limited to the above procedures.

A typical approach to synthesize the intermediate

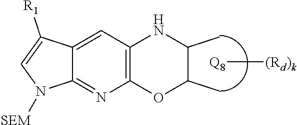

is described in Scheme 3.2 below.

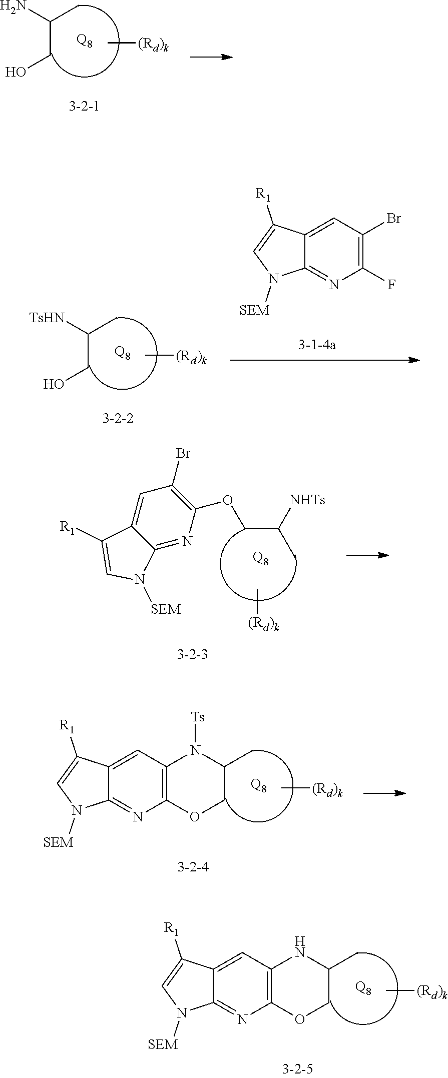

In Scheme 3-2, the starting material 3-2-1 can be prepared by conventional procedures using appropriate compounds and reagents. The starting material 3-2-1 can be converted to 3-2-2 readily, which further undergoes a SNAr reaction with 3-1-4a to give 3-2-3. After that, 3-2-3 undergoes an intramolecular coupling reaction to generate 3-2-4, which can be deprotected to afford 3-2-5.

Also, the target compounds can be synthesized by alternative methods but not limited to the above procedures.

A typical approach to synthesize the intermediate

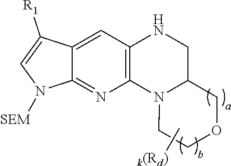

is described in Scheme 3-3 below.

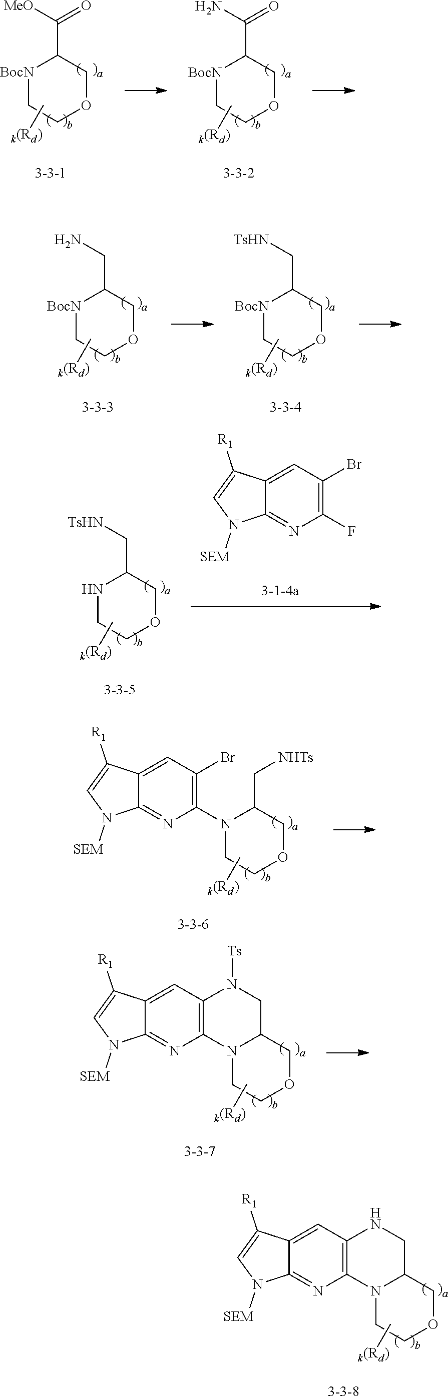

In Scheme 3-3, the starting material 3-3-1 can be converted to 3-3-3 through a two-step sequence of conventional reactions. After that, 3-3-3 can be converted to 3-3-4 readily, which can be deprotected to give 3-3-5. The reaction of 3-3-5 with 3-1-4a can generate 3-3-6, which can be converted to 3-3-7 via an intramolecular cyclization reaction. Finally, the deprotection of 3-3-7 can generate the target compounds 3-3-8.

Also, the target compounds can be synthesized by alternative methods but not limited to the above procedures.

A typical approach to synthesize the intermediate

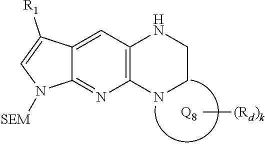

is described in Scheme 3-4 below.

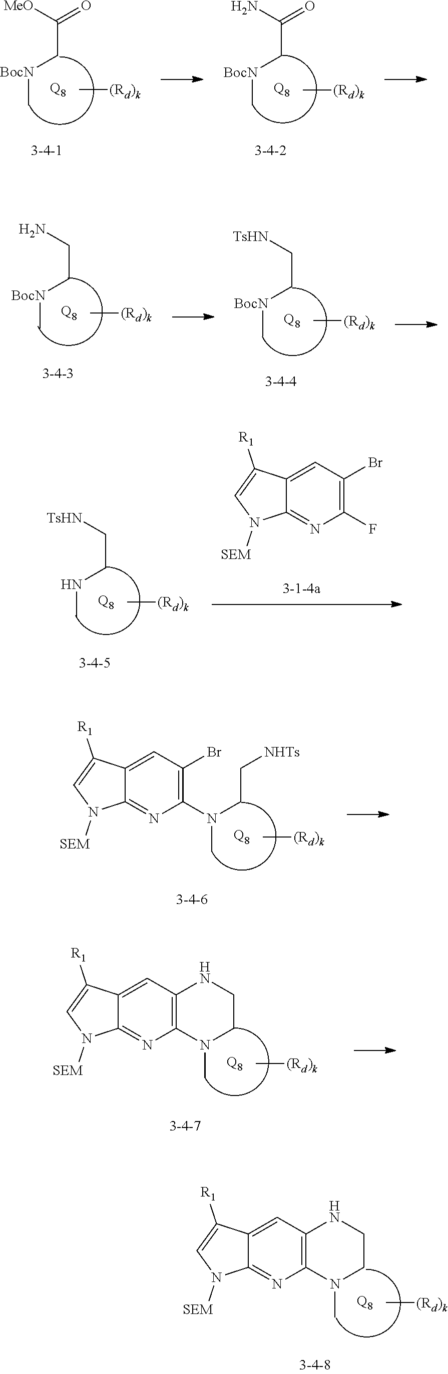

In Scheme 3-4, the starting material 3-4-1 can be converted to 3-4-3 through a two-step sequence of conventional reactions. After that, 3-4-3 can be converted to 3-4-4 readily, which can be deprotected to give 3-4-5. The reaction of 3-4-5 with 3-1-4a can generate 3-4-6, which can be converted to 3-4-7 via an intramolecular cyclization reaction. Finally, the deprotection of 3-4-7 can generated the target compounds 3-4-8.

Also, the target compounds can be synthesized by alternative methods but not limited to the above procedures.

A typical approach to synthesize the intermediate

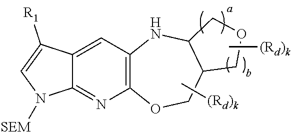

is described in Scheme 3-5 below.

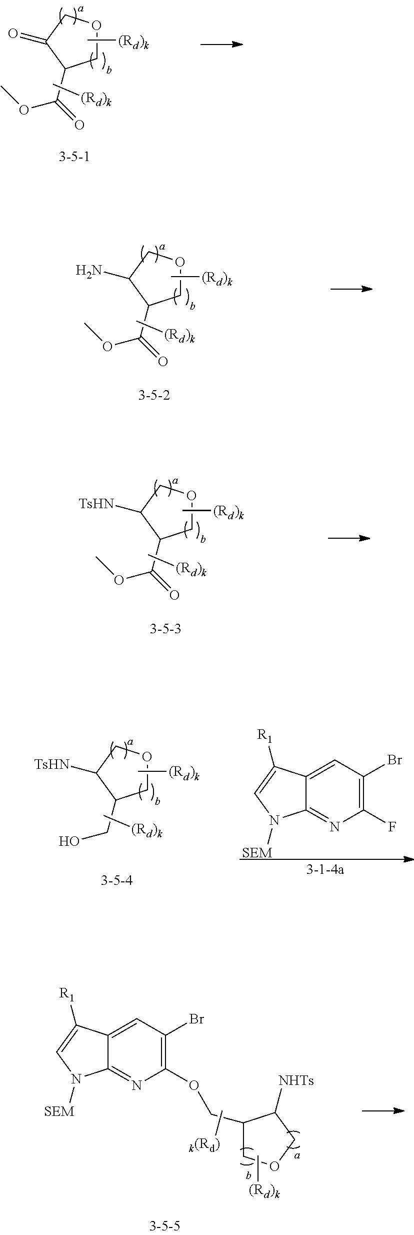

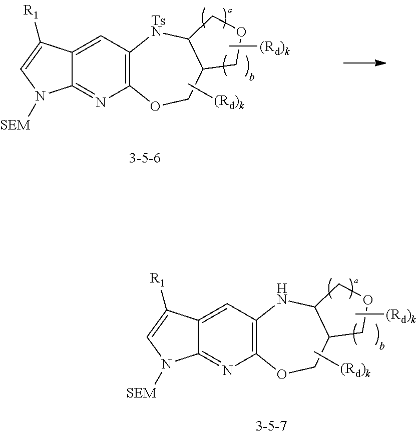

In Scheme 3-5, the starting material 3-5-1 can be converted to 3-5-2 via reductive amination reaction, and then 3-5-2 can be converted to 3-5-3 readily. After that, 3-5-3 can be reduced to 3-5-4. The reaction of 3-5-4 with 3-1-4a can generate 3-5-5, which can be converted to 3-5-6 via an intramolecular cyclization reaction. Finally, the deprotection of 3-5-6 can generate the target compounds 3-5-7.

Also, the target compounds can be synthesized by alternative methods but not limited to the above procedures.

A typical approach to synthesize the intermediate

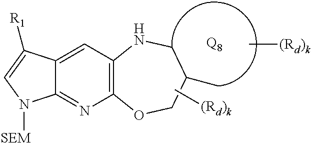

is described in 3-1-4

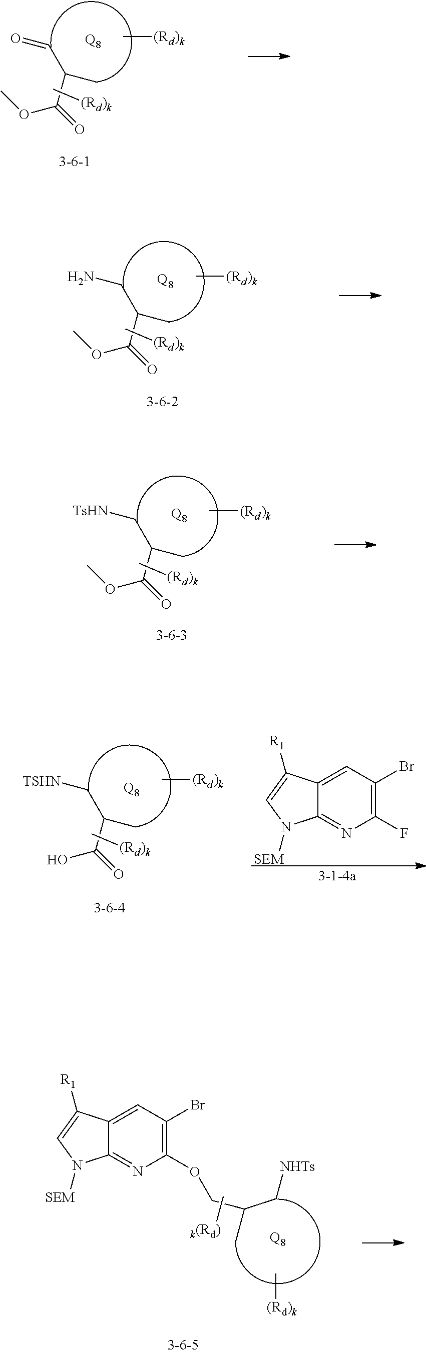

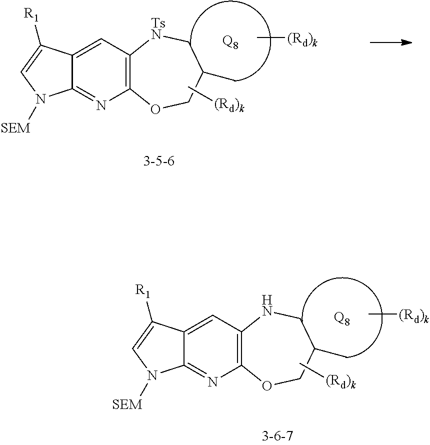

In Scheme 3-6, the starting material 3-6-1 can be converted to 3-6-2 via reductive amination reaction, and then 3-6-2 can be converted to 3-6-3 readily. After that, 3-6-3 can be reduced to 3-6-4. The reaction of 3-6-4 with 3-1-4a can generate 3-6-5, which can be converted to 3-6-6 via an intramolecular cyclization reaction. Finally, the deprotection of 3-6-6 can generate the target compounds 3-6-7.

Also, the target compounds can be synthesized by alternative methods but not limited to the above procedures.

A typical approach to synthesize the intermediate

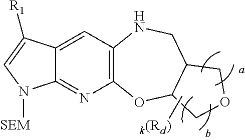

is described in Scheme 3-7 below.

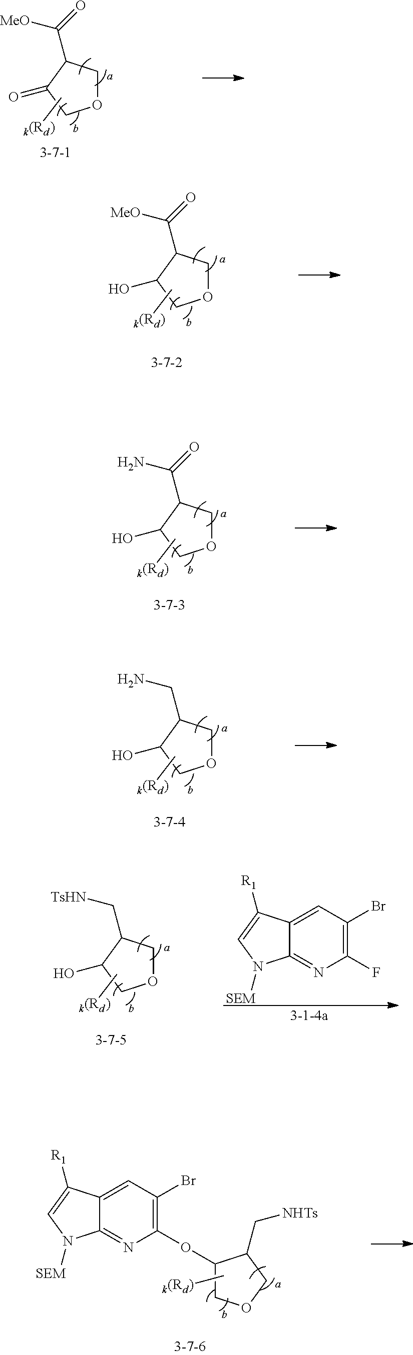

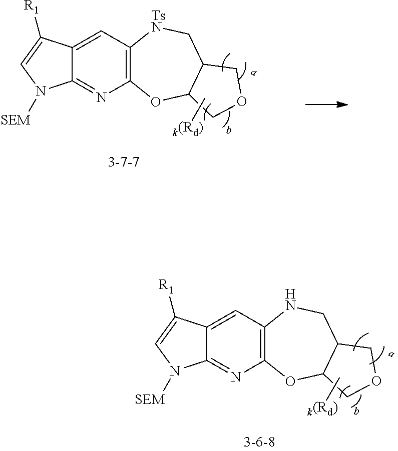

In Scheme 3-7, the starting material 3-7-1 can be reduced to give 3-7-2, and then 3-7-2 can be converted to 3-7-3 readily. After that, 3-7-3 can be converted to 3-7-5 through a two-step sequence of conventional reactions. The reaction of 3-7-5 with 3-1-4a can generate 3-7-6, which can be converted to 3-7-7 via an intramolecular cyclization reaction. Finally, the deprotection of 3-7-7 can generate the target compounds 3-7-8.

Also, the target compounds can be synthesized by alternative methods but not limited to the above procedures.

A typical approach to synthesize the intermediate

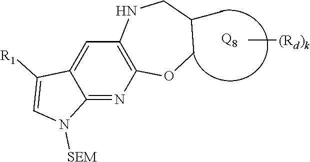

is described in Scheme 3-8 below.

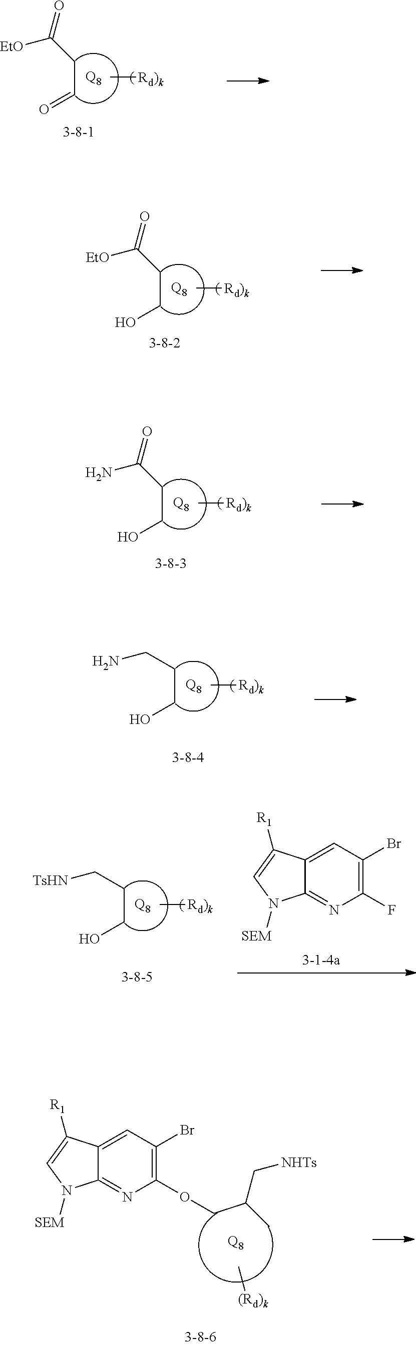

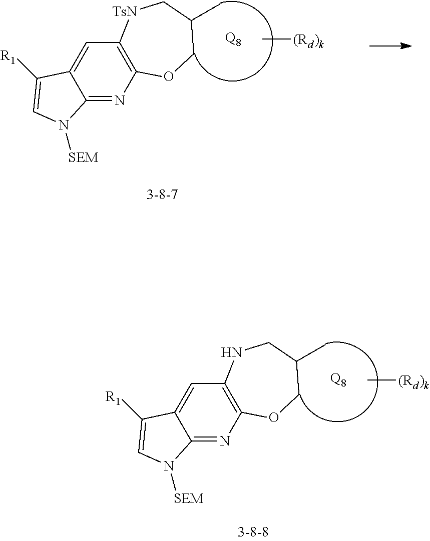

In Scheme 3-8, the starting material 3-8-1 can be reduced to give 3-7-2, and then 3-8-2 can be converted to 3-8-3 readily. After that, 3-8-3 is further converted to 3-8-5 through a two-step sequence of conventional reactions. The reaction of 3-8-5 with 3-1-4a can generate 3-8-6, which can be converted to 3-8-7 via an intramolecular cyclization reaction. Finally, the deprotection of 3-7-7 can generate the target compounds 3-8-8.

Also, the target compounds can be synthesized by alternative methods but not limited to the above procedures.

A typical approach to synthesize the intermediate

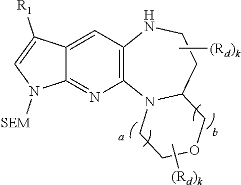

is described in Scheme 3-9 below.

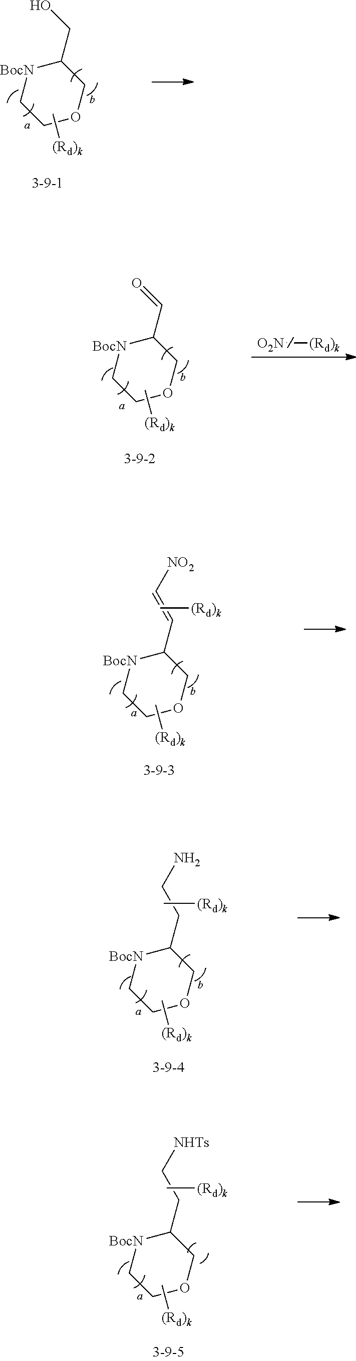

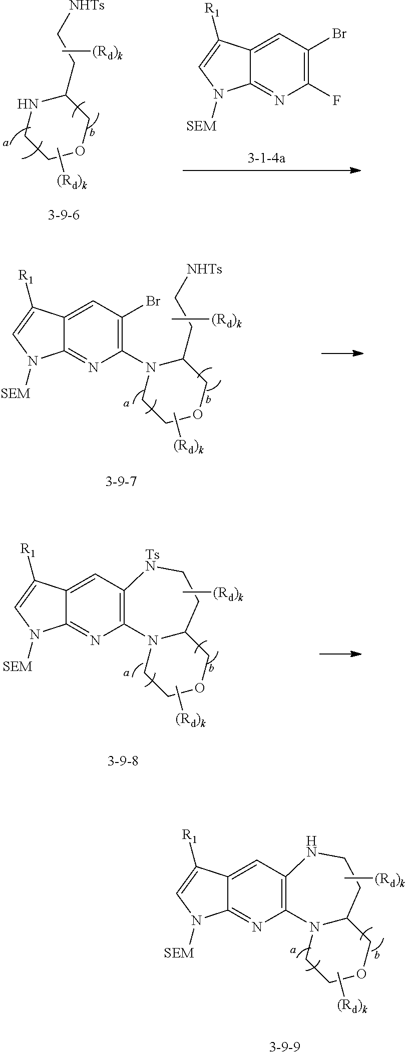

In Scheme 3-9, the starting material 3-9-1 can be oxidized to give 3-9-2, and then 3-9-2 can be converted to 3-9-3 readily. After that, the hydrogenation of 3-9-3 can give 3-9-4, which is further converted to 3-9-6 through a two-step sequence of conventional reactions. The reaction of 3-9-6 with 3-1-4a can generate 3-9-7, which can be converted to 3-9-8 via an intramolecular cyclization reaction. Finally, the deprotection of 3-9-8 can generate the target compounds 3-9-9.

Also, the target compounds can be synthesized by alternative methods but not limited to the above procedures.

A typical approach to synthesize the intermediate

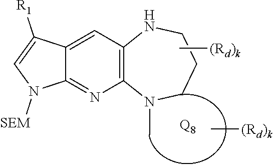

is described in Scheme 3-10 below.

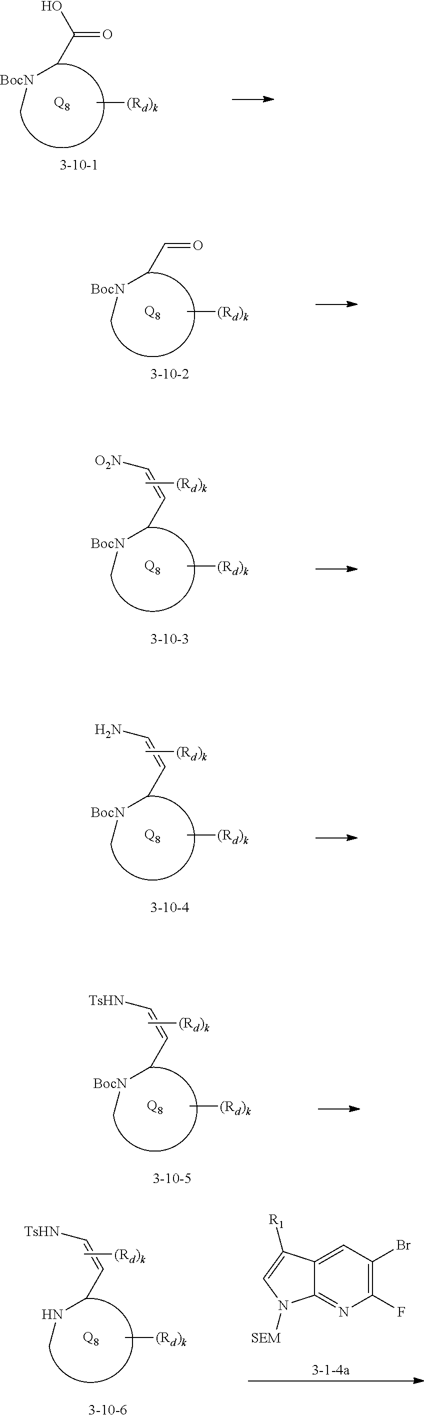

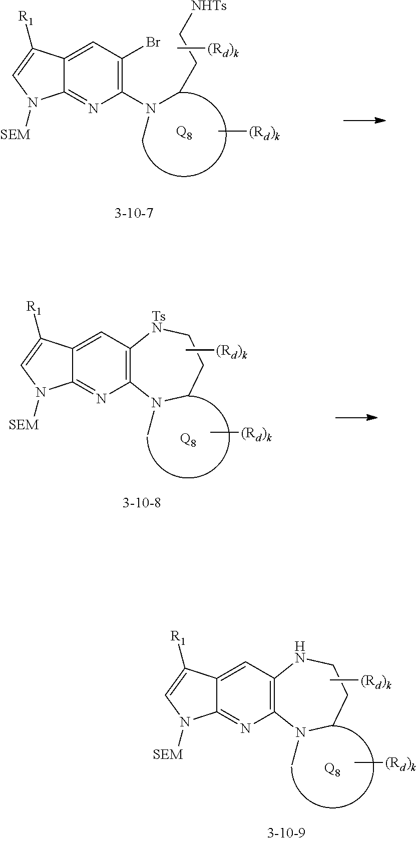

In Scheme 3-10, the starting material 3-10-1 can be reduced to give 3-10-2, and then 3-10-2 can be converted to 3-10-3 readily. After that, the hydrogenation of 3-10-3 can give 3-10-4, which can be converted to 3-10-6 through a two-step sequence of conventional reactions. The reaction of 3-10-6 with 3-1-4a can generate 3-10-7, which can be converted to 3-10.8 via an intramolecular cyclization reaction. Finally, the deprotection of 3-10-8 can generate the target compounds 3-10-9.

Also, the target compounds can be synthesized by alternative methods but not limited to the above procedures.

Similarly, the intermediate of

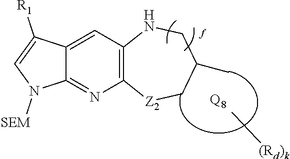

with different Z₂, f, k, R_{d} and Q₈ can be prepared by the method similar to the Scheme 3-1 to 3-10 by using appropriate staring materials, and intermediates.

Similarly, the compounds of

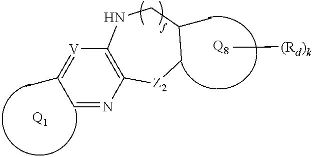

can be prepared by schemes similar to the Scheme 3-1 to 3-10 by using appropriate staring materials and intermediates.

An approach to synthesize compounds of

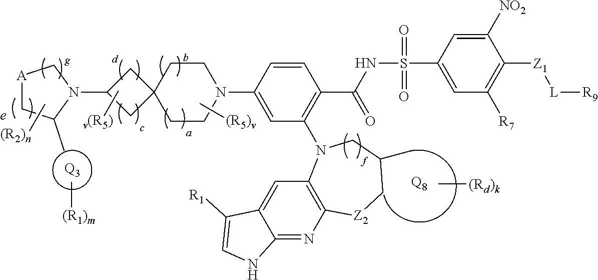

is described in Scheme A:

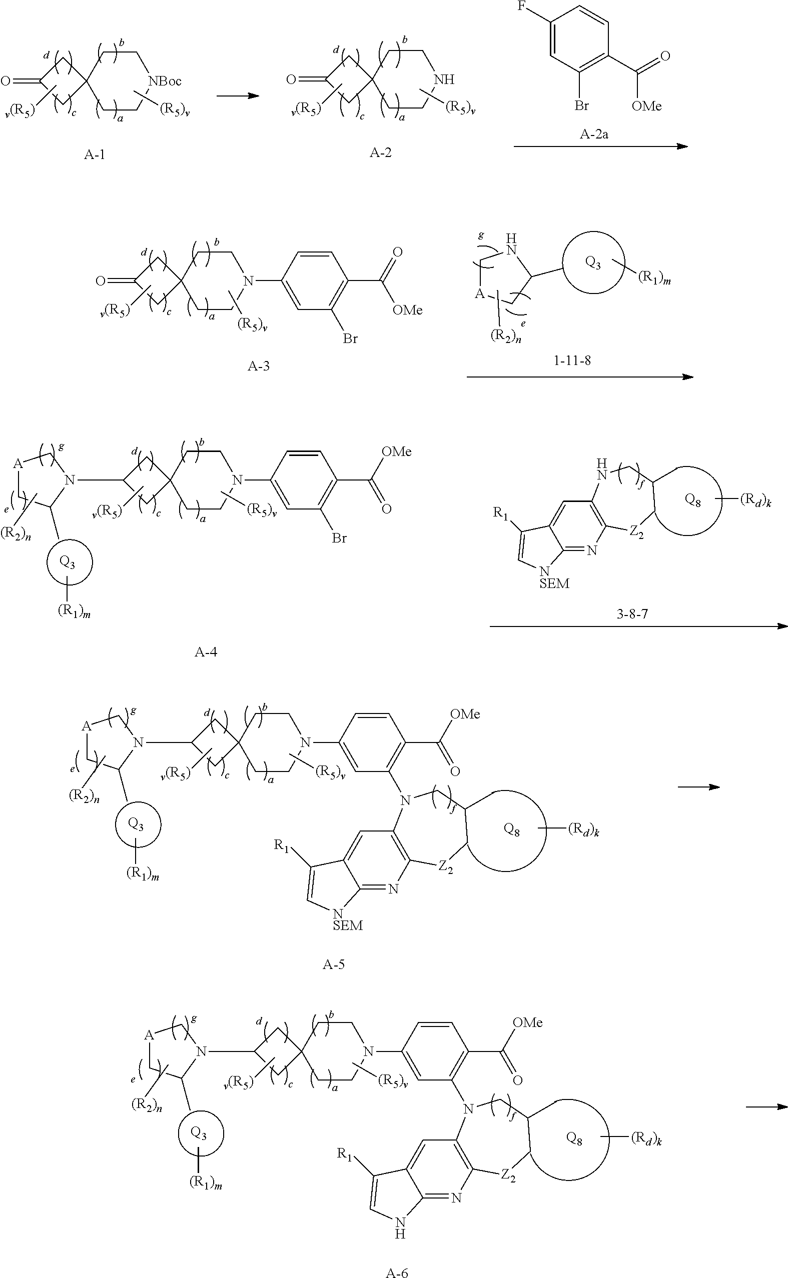

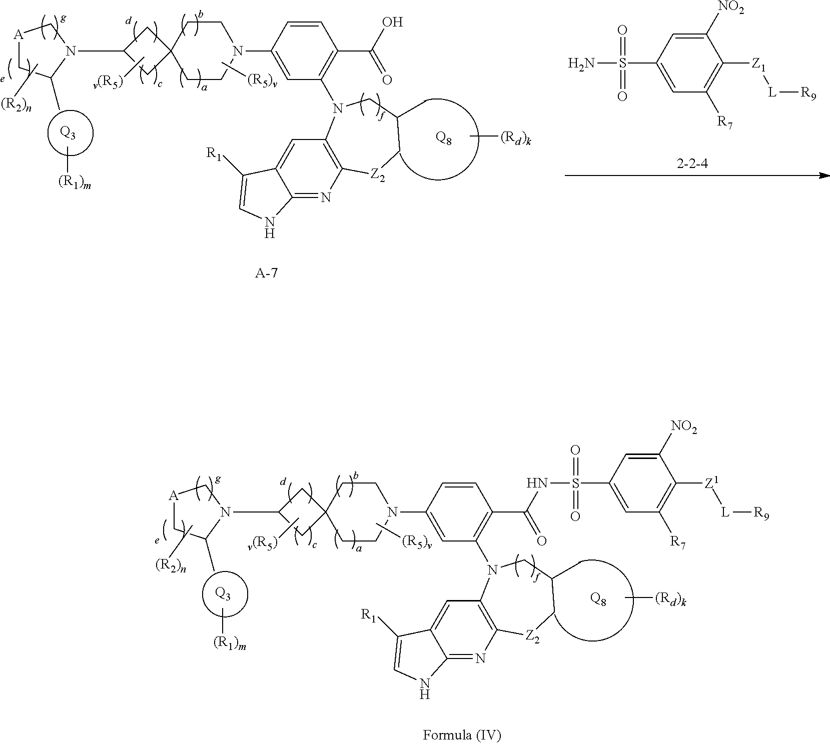

In Scheme A, the starting material A-1 can be prepared by conventional procedures using appropriate compounds and reagents. The starting material A-1 can be converted to A-2 readily, which further undergoes a SNAr reaction with A-2a to give A-3. After that, the intermediate A-3 can be be converted to A-4 via a reductive amination reaction with 1-11-8, and A-4 undergoes a coupling reaction with 3-8-7 to give A-5. Next, the deprotection of A-5 can afford A-6, which can be hydrolyzed to generate A-7. Finally, A-7 undergoes a condensation reaction with 2-2-4 to afford the target compounds.

Also, the target compounds can be synthesized by alternative methods but not limited to the above procedures.

An approach to synthesize compounds of

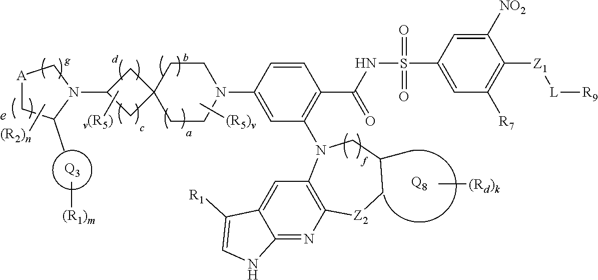

is described in Scheme B:

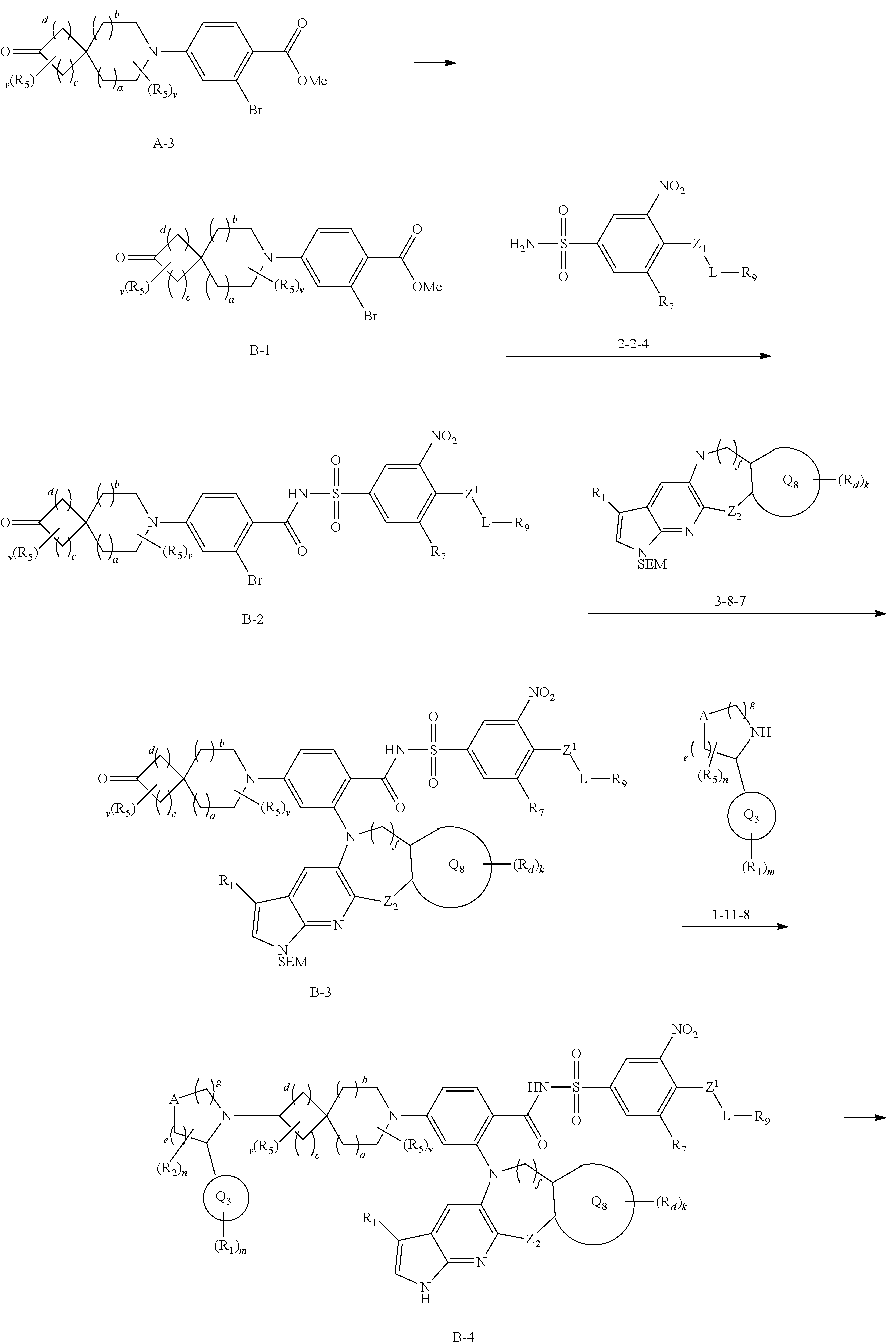

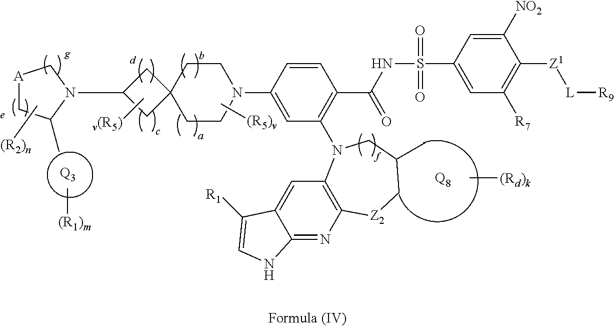

In Scheme B, the starting material A-3 can be prepared by conventional procedures using appropriate compounds and reagents. The starting material A-3 can be hydrolyzed to generate B-1 readily. After that, the intermediate B-1 undergoes a condensation reaction with 2-2-4 to give B-2, which further couples with 3-8-7 to give B-3. The intermediate B-3 can be converted to B-4 via a reductive amination reaction with 1-11-8. Finally, the deprotection of B-4 can generate the target compounds.

Also, the target compounds can be synthesized by alternative methods but not limited to the above procedures.

An approach to synthesize compounds of

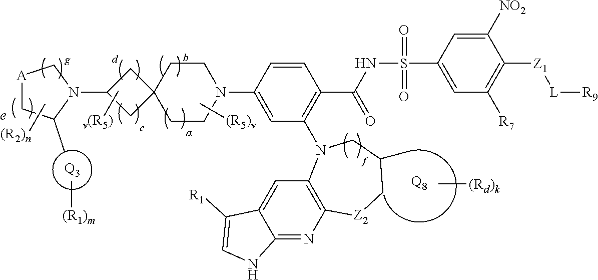

is described in Scheme C:

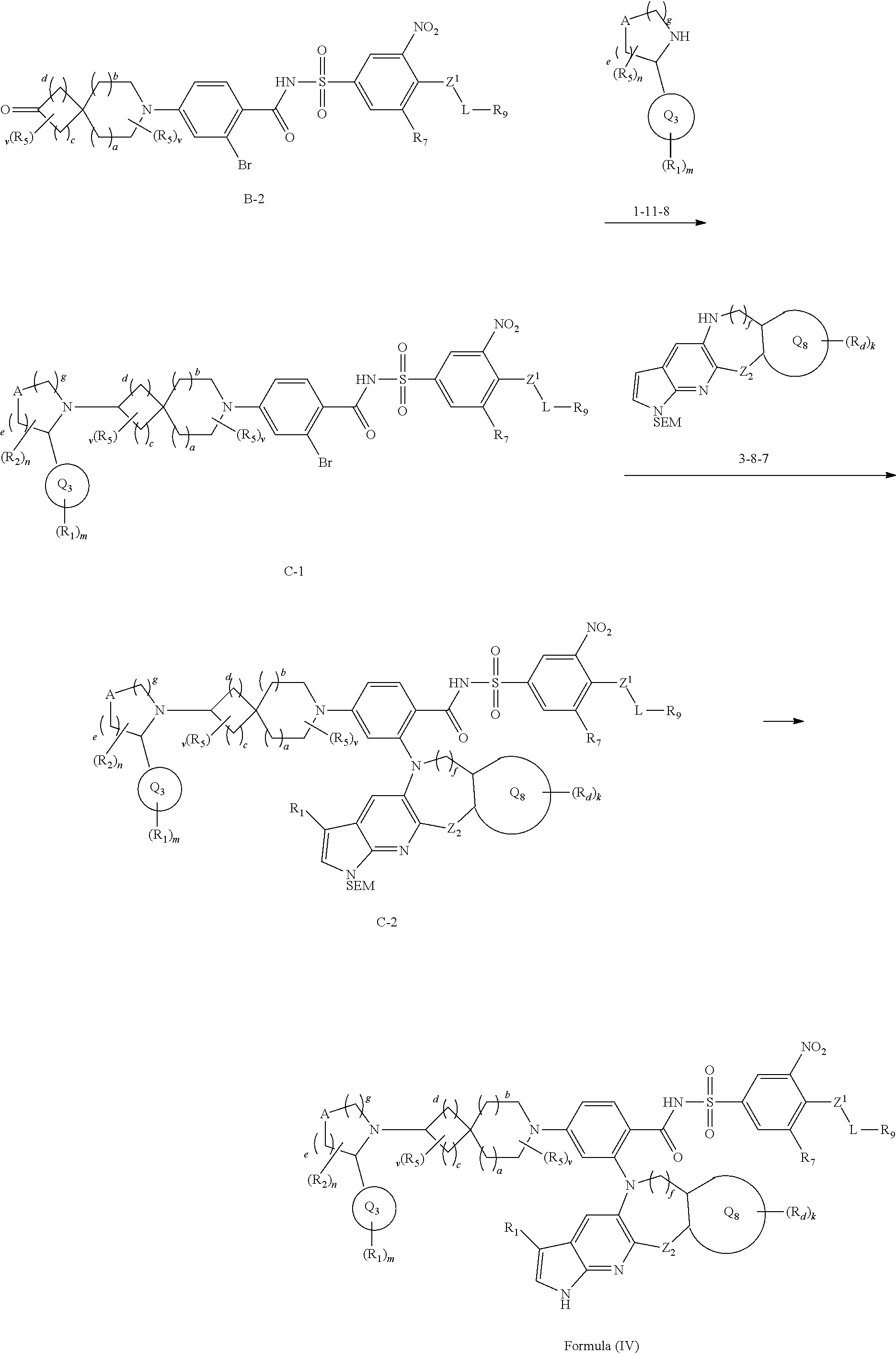

In Scheme C, the starting material B-2 can be prepared by conventional procedures using appropriate compounds and reagents. The starting material B-2 can be converted to C-1 via a reductive amination reaction with 1-11-8, and then C-1 can can undergoes a coupling reaction with 3-8-7 to give C-2. Finally, the deprotection of C-2 can generate the target compounds.

Also, the target compounds can be synthesized by alternative methods but not limited to the above procedures.

An approach to synthesize compounds of

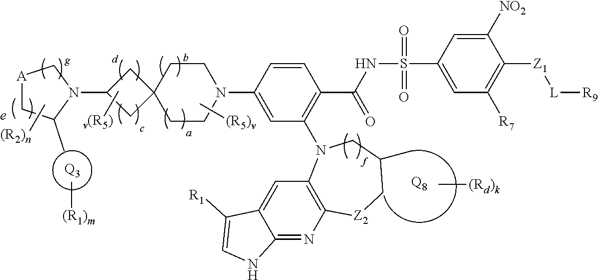

is described in Scheme D:

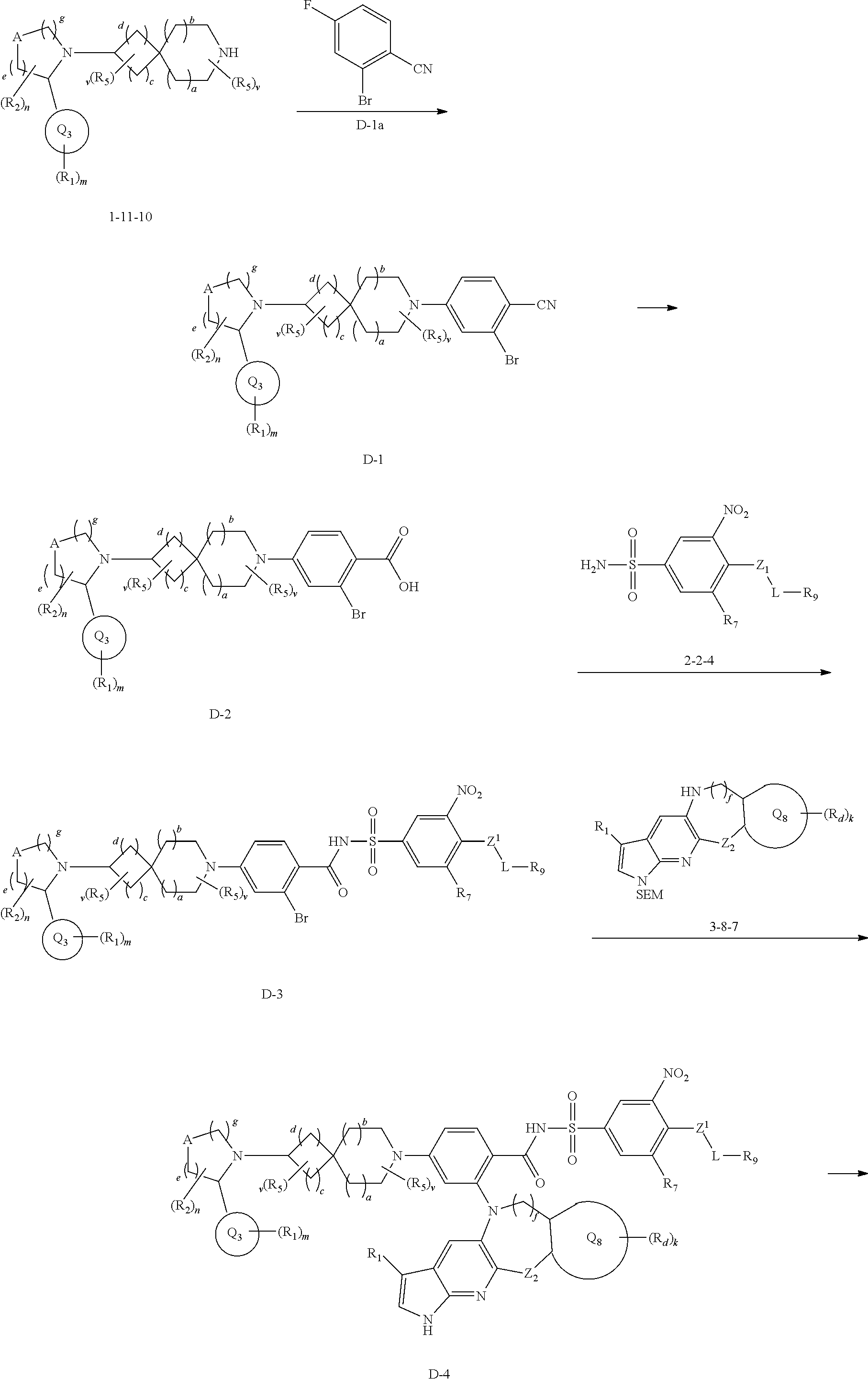

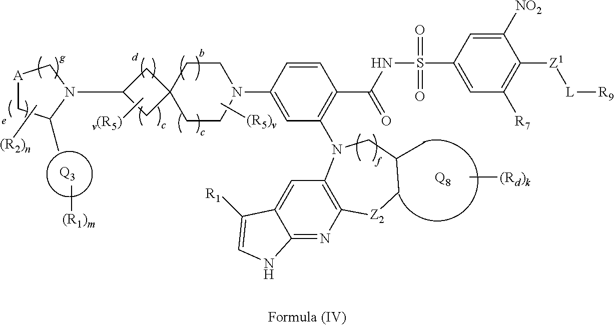

In Scheme D, the starting material 1-11-10 can be prepared by conventional procedures using appropriate compounds and reagents. The starting material 1-11-10 can be converted to D-1 readily, which can be hydrolyzed to generate D-2. After that, the intermediate D-2 undergoes a condensation reaction with 2-2-4 to give D-3, which further couples with 3-8-7 to give D-4. Finally, the deprotection of D-4 can give the target compounds.

Also, the target compounds can be synthesized by alternative methods but not limited to the above procedures.

An approach to synthesize compounds of

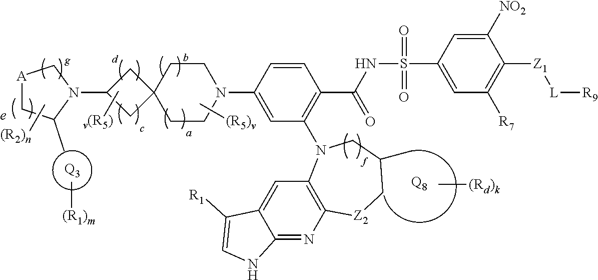

is described in Scheme E:

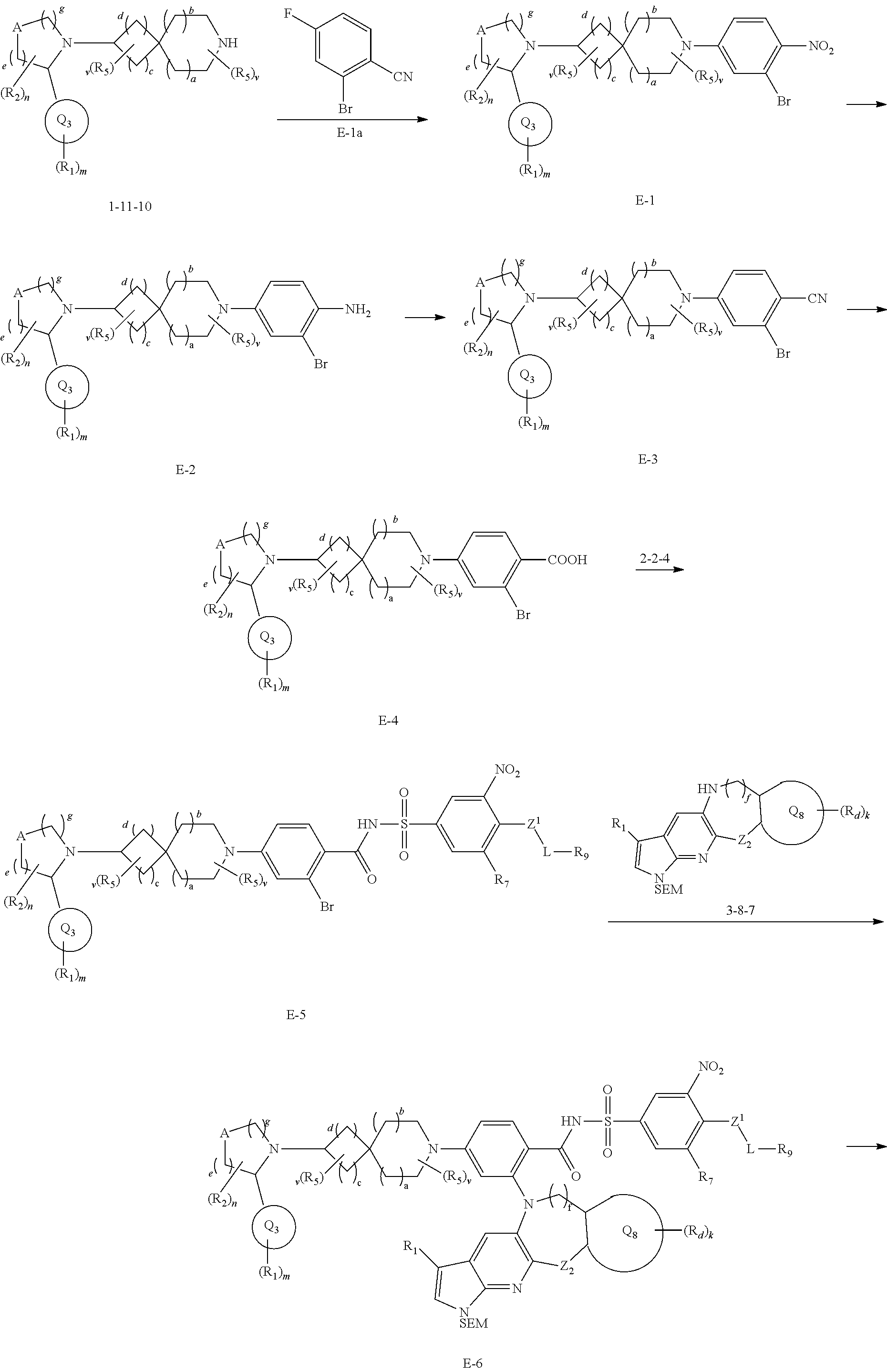

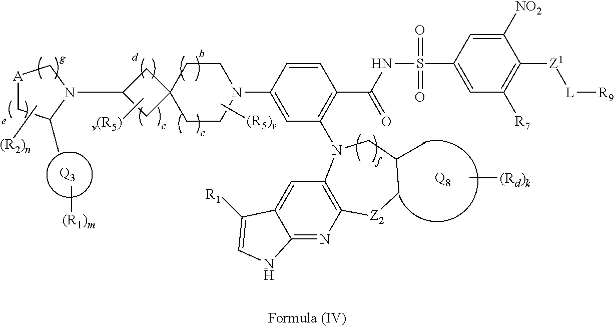

In Scheme E, the starting material 1-11-10 can be prepared by conventional procedures using appropriate compounds and reagents. The starting material 1-11-10 can be converted to E-1 readily, which can be reduced to generate E-2. After that, the intermediate E-2 undergoes a Sandmeyer reaction to give E-3, which can be hydrolyzed to generate E-4. The condensation of intermediate E-4 with 2-2-4 can give E-5. Next, E-5 couples with 3-8-7 to give E-6. Finally, the deprotection of E-6 can give the target compounds.

Also, the target compounds can be synthesized by alternative methods but not limited to the above procedures.

An approach to synthesize compounds of

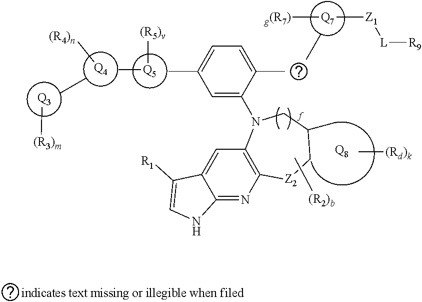

is described in Scheme F:

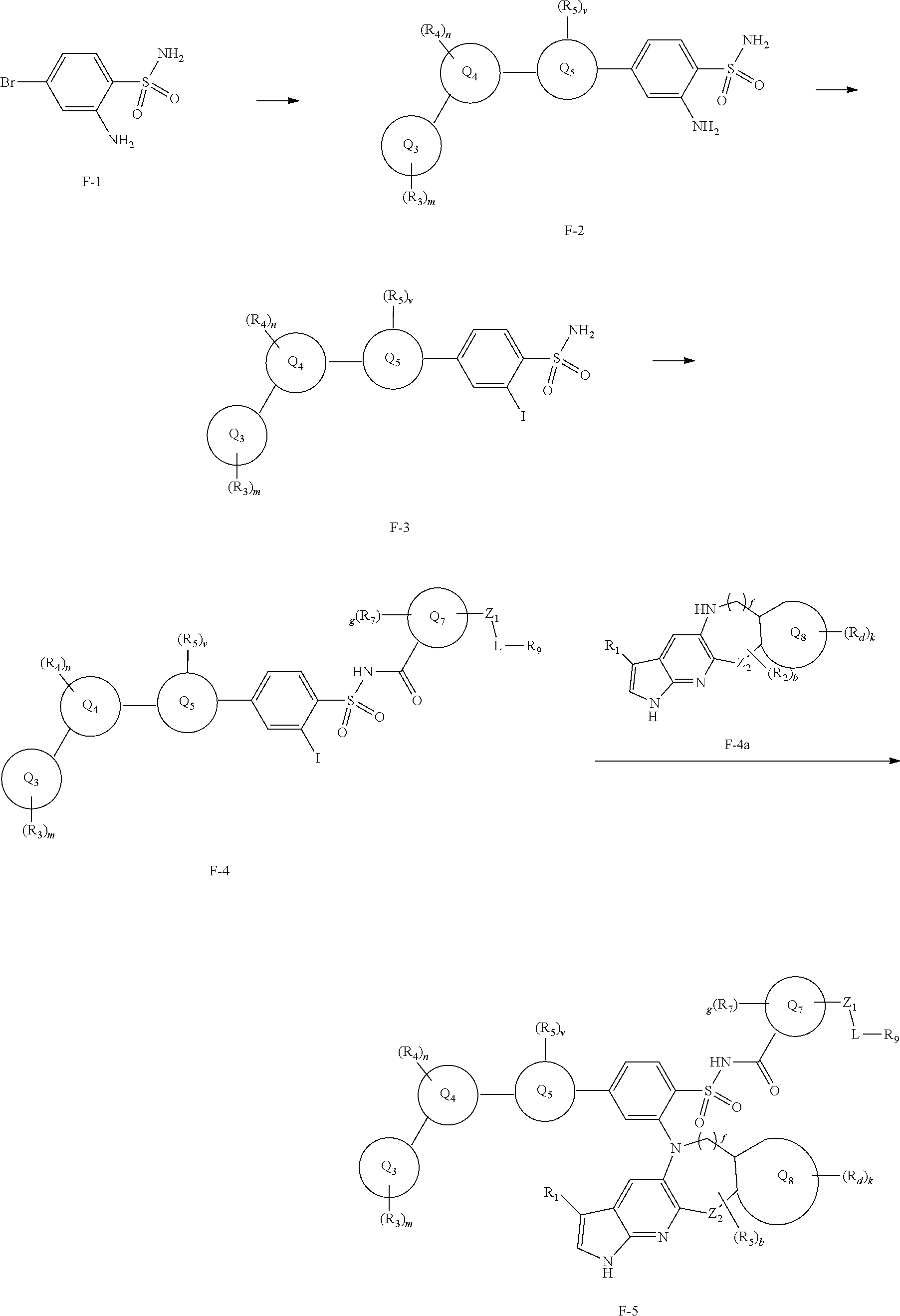

In Scheme F, the starting material F-1 can be prepared by conventional procedures using appropriate compounds and reagents. The starting material F-1 can be converted to F-2 via a coupling reaction, which is further converted to F-3 through Sandmeyer reaction. After that, the condensation of intermediate F-3 with the corresponding carboxylic acid can give F-4. Finally, the intermediate F-4 can couple with F-4a to generate the target compounds F-5.

Also, the target compounds can be synthesized by alternative methods but not limited to the above procedures.

An approach to synthesize of target compounds of

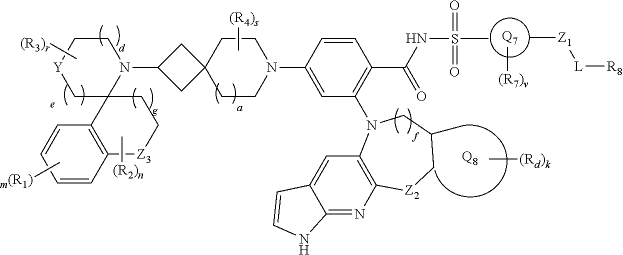

is described in Scheme G:

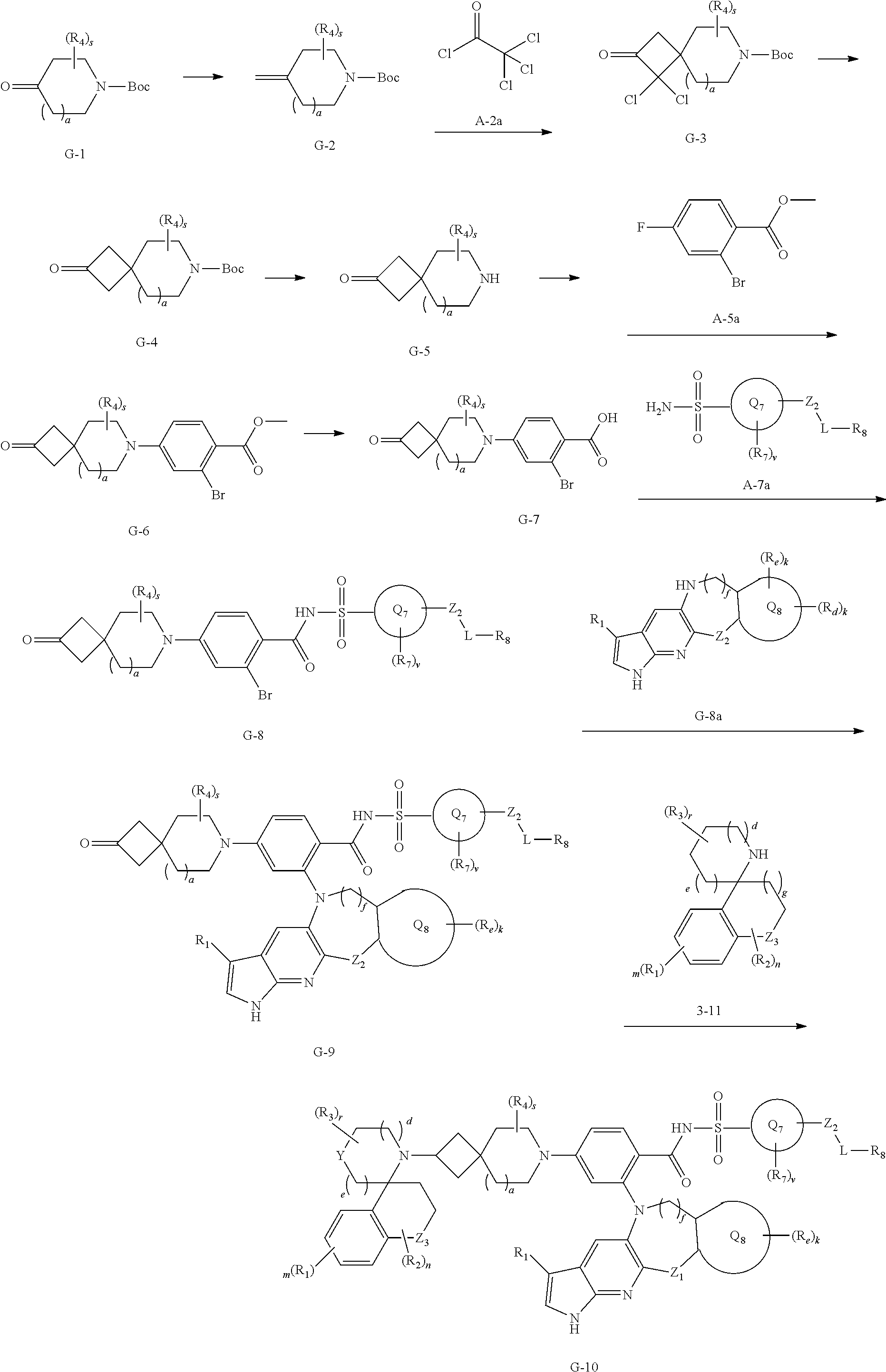

In Scheme G, the starting material G-1 can be prepared by conventional procedures using appropriate compounds and reagents. The starting material G-1 can be converted to G-2 via a general Wittig reaction, which further undergoes Staudinger ketene cycloaddition with G-2a to give G-3. After that, the dichloride G-3 can be redacted to G-4 by a suitable reagent, and then deprotection of G-4 can generate intermediate G-5. The intermediate G-5 undergoes a coupling reaction with G-5a to give G-6, which can be hydrolyzed to afford G-7. Next, the intermediate G-7 can react with G-7a to give G-8, which can couple with G-8a to give G-9. Finally, G-9 can undergo a reductive amination with 3.11 to afford the target compounds G-10.

Also, the target compounds can be synthesized by alternative methods but not limited to the above procedures.

Similarly, the compounds of

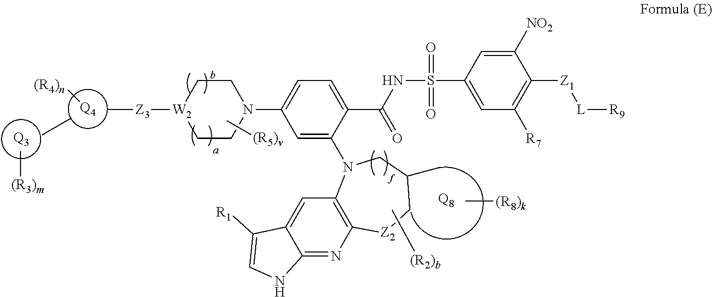

can be prepared by schemes similar to the Scheme A-Scheme G by using appropriate staring materials and intermediates.

Similarly, the compounds of

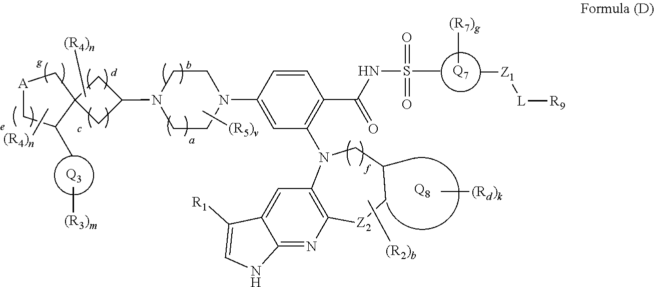

can be prepared by schemes similar to the Scheme A-Scheme G by using appropriate staring materials and intermediates.

Similarly, the compounds of

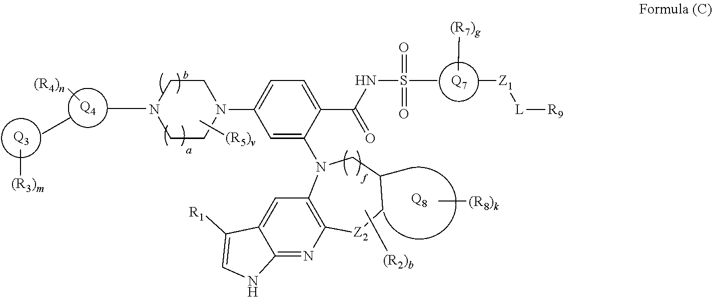

can be prepared by schemes similar to the Scheme A-Scheme G by using appropriate staring materials and intermediates.

Similarly, the compounds of

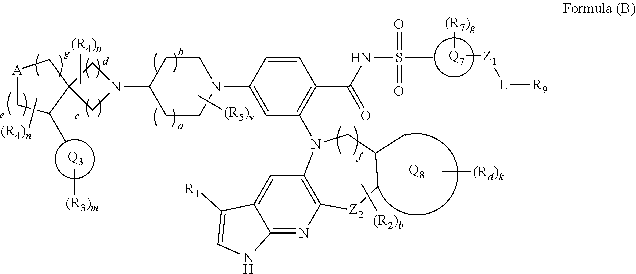

can be prepared by schemes similar to the Scheme A-Scheme G by using appropriate staring materials and intermediates.

Similarly, the compounds of

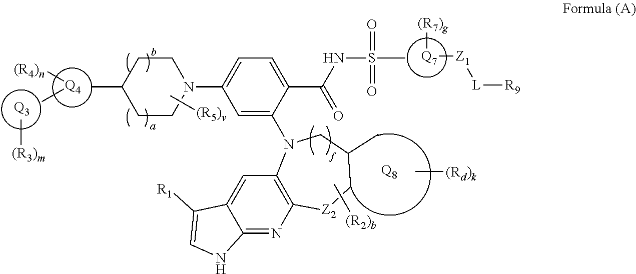

can be prepared by schemes similar to the Scheme A-Scheme G by using appropriate staring materials and intermediates.

Similarly, the compounds of

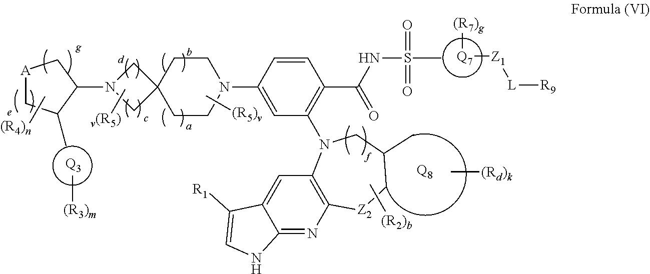

can be prepared by schemes similar to the Scheme A-Scheme G by using appropriate staring materials and intermediates.

Similarly, the compounds of

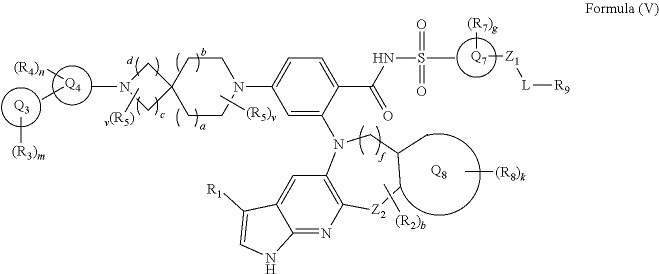

can be prepared by schemes similar to the Scheme A-Scheme G by using appropriate staring materials and intermediates.

Similarly, the compounds of

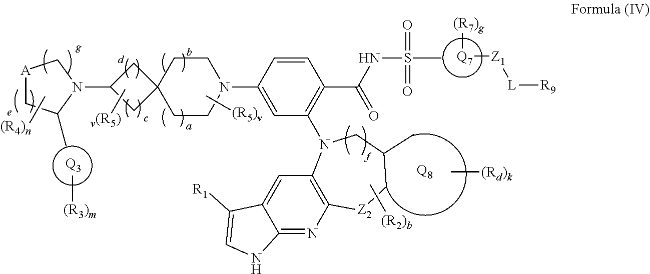

can be prepared by schemes similar to the Scheme A-Scheme G by using appropriate staring materials and intermediates.

Similarly, the compounds of

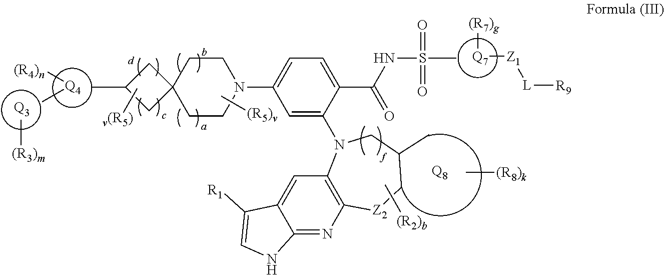

can be prepared by schemes similar to the Scheme A-Scheme G by using appropriate staring materials and intermediates.

Similarly, the compounds of

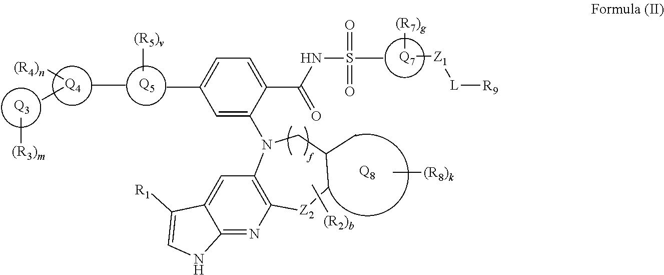

can be prepared by schemes similar to the Scheme A-Scheme G by using appropriate staring materials and intermediates.

Similarly, the compounds of

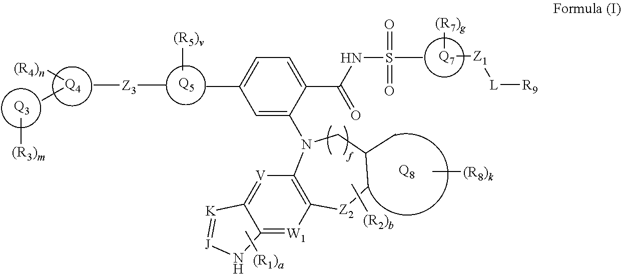

can be prepared by schemes similar to the Scheme A-Scheme G by using appropriate staring materials and intermediates.

Similarly, the compounds of

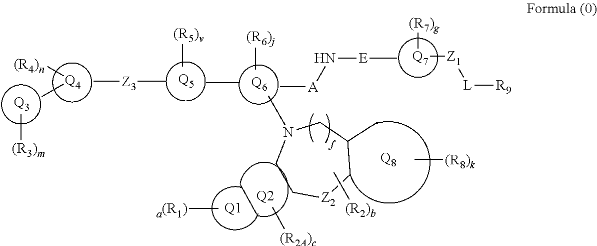

can be prepared by schemes similar to the Scheme A-Scheme G by using appropriate staring materials and intermediates.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system.

Where LC/MS data are presented, analyses were performed using an Applied Biosystems API-100 mass spectrometer and Shimadzu SCL-10A LC column:

### Example INT_1: Preparation of 1-((4′-chloro-5,5-dimethyl-3,4,5,6-tetrahydro-[1,1′-biphenyl]-2-yl)methyl)piperazine

Synthesis of 2-bromo-4,4-dimethylcyclohex-1-enecarbaldehyde: A solution of anhydrous CHCl₃ (57 mL) and anhydrous N, N-dimethylformamide (9 mL) were cooled to 3° C. (internal temperature) under nitrogen before phosphorus tribromide (10 mL, 0.1 mol) was introduced dropwise at a rate so that the reaction was maintained at 3° C. After the addition was complete the reaction was allowed to warm slowly to 10° C. and then the temperature was raised to 70° C. where it was maintained for 30 min. The reaction was cooled to 25° C. and 3,3-dimethylcyclohexanone 1 (5 g, 0.04 mol) was added slowly over 20 min. After the addition was complete the reaction was warmed to 70° C. and it was stirred for 1.5 h. The mixture was then cooled to 0° C. and a solution of 4 M sodium acetate (53 mL) was added slowly. The pH of the resulting solution was adjusted to 7 using a solution of 5 M NaOH and the mixture was then extracted with heptanes (3×100 mL). The combined organic fractions were dried (Na₂SO₄), filtered and concentrated under reduced pressure to give 2-bromo-4,4-dimethylcyclohex-1-enecarbaldehyde (4 g, 49%) as a yellow oil.

Synthesis of 2-(4-chlorophenyl)-4,4-dimethylcyclohex-1-enecarbaldehyde: To a degassed solution of 2-bromo-4,4-dimethylcyclohex-1-enecarbaldehyde 2 (5 g, 0.023 mol) and 4-chlorophenyl boronic acid (3.6 g, 0.023 mol) in 1,4-dioxane (50 mL) at 25° C. was added a solution of 2.0 M Na₂CO₃ (20.4 mL). Nitrogen was bubbled through the mixture for 2 min and then PdCl₂(dqpf) (0.5 g) was added. The reaction flask was heated to 120° C. where it was maintained for 3 h. After this time the suspension was cooled to 25° C. and filtered through Celite. The collected solids were washed with additional dichloromethane and the combined filtrate and washings were concentrated under reduced pressure. Purification by column chromatography on silica with ethyl acetate/petroleum ether=1: 20 gave 2-(4-chlorophenyl)-4,4-dimethylcyclohex-1-enecarbaldehyde (3 g, 53%) as a white solid. LC-MS (ESI, m/z) M+1: 249.

Synthesis of (2-(4-chlorophenyl)-4,4-dimethylcyclohex-1-enyl)methanol: A solution of 2-(4-chlorophenyl)-4,4-dimethylcyclohex-1-enecarbaldehyde 3 (20 g, 80.6 mmol) in MeOH (100 mL) was cooled to 0° C., NaBH₄ (3.1g, 80.6 mmol) was added portionwise to the reaction at a rate so that the reaction was maintained at 0˜5° C. After added, the mixture was stirred for 1 h at 0° C. Water was added slowly to the mixture and extracted with ethyl acetate (3×200 mL), the organic layer was washed with brine and dried Na₂SO₄, filtered and concentrated under reduced pressure to give (2-(4-chlorophenyl)-4,4-dimethylcyclohex-1-enyl)methanol (15 g, 75%) as a white solid. LC-MS (ESI, m/z) M+1: 233.

Synthesis of 1-(2-(bromomethyl)-5,5-dimethylcyclohex-1-enyl)-4-chlorobenzene: A solution of (2-(4-chlorophenyl)-4,4-dimethylcyclohex-1-enyl)methanol (15 g, 0.06 mol) and in Et₂O (300 mL) was cooled to 0° C. before phosphorus tribromide (7.5 mL) was added dropwise to the mixture, after added, the mixture was stirred for 1 h at 0° C. for 90 minutes. The reaction mixture was added water before being extracted with ethyl acetate. The organic layer was washed with a saturated NaHCO₃ solution and brine and dried Na₂SO₄, filtered and concentrated under reduced pressure to give 1-(2-(bromomethyl)-5,5-dimethylcyclohex-1-enyl)-4-chlorobenzene (18 g, 96%) as a colorless oil.

Synthesis of tert-butyl 4-((2-(4-chlorophenyl)-4,4-dimethylcyclohex-1-enyl)methyl)piperazine-1-carboxylate: To a solution of 1-bromo-2-(bromomethyl)-5,5-dimethylcyclohex-1-ene (21 g, 0.067 mol) and tert-butyl piperazine-1-carboxylate (12.4 g, 0.067 mol) in dichloromethane (200 mL) at 25° C. was added TEA (12.2 g, 0.12 mol). The reaction was stirred for 2 h. The reaction mixture was concentrated under reduced pressure to give the crude product. Purification by column chromatography on silica with petroleum ether/ethyl acetate=20:1 to provided tert-butyl 4-((2-(4-chlorophenyl)-4,4-dimethylcyclohex-1-enyl)methyl)piperazine-1-carboxylate (21 g, 75%).

Synthesis of 1-((2-(4-chlorophenyl)-4,4-dimethylcyclohex-1-enyl)methyl)piperazine hydrogen chloride: To a solution of tert-butyl 4-((2-(4-chlorophenyl)-4,4-dimethylcyclohex-1-enyl)methyl)piperazine-1-carboxylate 6 (30 g, 0.072 mol) in MeOH (20 mL) was added conc.HCl (50 mL). The reaction was stirred for 24 hours and then concentrated under reduced pressure. A saturated solution of Na₂CO₃ was added to adjust the pH to 8-9 and the mixture was extracted with dichloromethane (2×200 mL). The combined extracts were washed with brine, dried (Na₂SO₄), filtered and concentrated under reduced pressure. The oil product was treated with MeOH/HCl (g) (3.0 M, 500 mL) and stirred for 1 hour, then concentrated under reduced pressure to get the product 1-((2-(4-chlorophenyl)-4,4-dimethylcyclohex-1-enyl)methyl)piperazine hydrogen chloride (23 g, 83%). LC-MS (ESI, m/z) M+1: 319. ¹HNMR (400 MHz, DMSO-d₆) δ 11.51 (s, 1H), 9.60 (s, 1H), 9.18 (s, 1H), 7.45 (d, J=8.2 Hz, 2H), 7.15 (d, J=8.0 Hz, 2H), 3.43 (s, 8H), 2.84 (s, 2H), 2.39 (s, 2H), 2.03 (s, 2H), 1.45 (t, J=6.0 Hz, 2H), 0.96 (s, 6H).

### Example INT_2: Preparation of 3-nitro-4-(((tetrahydro-2H-pyran-4-yl)methyl)amino)benzenesulfonamide

To a 500 mL three-neck flask equipped with a mechanical stirrer were charged the 4-chloro-3-nitrobenzenesulfonamide (23.7 g, 100 mmol), DIPEA (12.9 g, 100 mmol), (tetrahydro-2H-pyran-4-yl)methanamine (11.5 g, 100 mmol) and acetonitrile (200 mL). The reaction mixture was adjusted to an internal temperature of 80° C. and agitated for no less than 12 hours. The product solution was cooled down to 40° C. and agitated for no less than 1 hour until precipitation observed. The product slurry was further cooled to 20° C. Water (80 mL) was slowly charged over no less than 1 hour, and the mixture cooled to 10° C. and agitated for no less than 2 hours before collected by filtration. The wet cake was washed with 1:1 mix of acetonitrile:water (40 mL). The wet cake was rinsed with water (80 mL) at 40° C. for no less than 1 hour before collected by filtration. The wet cake was rinsed with water (20 mL), and dried at 75° C. under vacuum to give the 3-nitro-4-(((tetrahydro-2H-pyran-4-yl)methyl)amino)benzenesulfonamide (24.5 g, 78%) as an orange solid. ¹HNMR (400 MHz, DMSO-d₆) δ 8.60 (t, J=5.9 Hz, 1H), 8.48 (d, J=2.2 Hz, 1H), 7.84 (dd, J=9.2, 2.0 Hz, 1H), 7.54-7.18 (m, 3H), 3.86 (dd, J=11.3, 3.2 Hz, 2H), 3.35 (s, 2H), 3.27 (t, J=10.9 Hz, 2H), 1.92 (ddd, J=11.2, 7.4, 3.9 Hz, 1H), 1.62 (d, J=11.4 Hz, 2H), 1.27 (qd, J=12.3, 4.4 Hz, 2H).

### Example INT_3: Preparation of 4-[[(4-fluorooxan-4-yl)methyl]amino]-3-nitrobenzene-1-sulfonamide

Into a 50-mL round-bottom flask, were placed (4-fluorooxan-4-yl)methanamine hydrochloride (500 mg, 2.95 mmol, 1.0 eq), 4-fluoro-3-nitrobenzene-1-sulfonamide (650 mg, 2.95 mmol, 1.0 eq), tetrahydrofuran (15 mL), Cs₂CO₃ (2.8 g, 8.59 mmol, 3.0 eq). The resulting solution was stirred for 14 h at 50° C. in an oil bath. The reaction mixture was cooled to room temperature. The resulting mixture was filtered and concentrated under vacuum. The residue was applied onto a silica gel column with ethyl acetate/petroleum ether (4:1). This resulted in 650 mg (66%) of 4-[[(4-fluorooxan-4-yl)methyl]amino]-3-nitrobenzene-1-sulfonamide as a yellow solid. LC-MS (ESI, m/z) M+1: 334. ¹HNMR (300 MHz, DMSO-d₆): δ 8.58 (t, J=6.3 Hz, 1H), 8.49 (d, J=2.1 Hz, 1H), 7.90-7.80 (m, 1H), 7.44 (d, J=9.3 Hz, 1H), 7.34 (s, 2H), 3.87-3.70 (m, 4H), 3.61-3.50 (m, 2H), 1.95-1.70 (m, 4H).

### Example INT_4: Preparation of (S)-4-((1,4-dioxan-2-yl)methylamino)-3-nitrobenzenesulfonamide

Synthesis of (R)-1-chloro-3-(2-chloroethoxy)propan-2-ol: (R)-2-(chloromethyl)oxirane (500.0 g, 5.4 mol, 1.0 eq) was slowly added to a stirred solution of 2-chloroethanol (870.0, 10.8 mol, 2.0 eq) and BF₃·Et₂O (38.0 g, 27 mmol, 0.05 eq) at 45° C. The reaction mixture was heated on an oil bath for 3 h at 45° C. The reaction mixture was cooled to 25° C. and Diethyl ether (100 mL) was added to this solution. The organic layer was washed with water (2×300 mL), dried over magnesium sulfate, and concentrated to yield a light brown liquid (R)-1-chloro-3-(2-chloroethoxy)propan-2-ol (800.0 g). ¹HNMR (300 MHz, DMSO-d₆) δ 3.85-3.47 (m, 9H).

Synthesis of (R)-2-((2-chloroethoxy)methyl)oxirane: (R)-1-chloro-3-(2-chloroethoxy)propan-2-ol (800.0 g, crude, 4.7 mol, 1.0 eq) was added dropwise to a stirred solution of NaOH (465.0 g, 11.6 mol, 2.5 eq) in water (500 mL) on an ice-bath. The ice-bath was immediately removed after addition of (R)-1-chloro-3-(2-chloroethoxy)propan-2-ol. After stirring 2 h at 25° C., diethyl ether (1.5 L) and water (500 mL) were added. The organic layer was washed with water (1×50 mL), dried over sodium sulfate, and concentrated to give a light brown liquid (R)-2-((2-chloroethoxy)methyl)oxirane (400.0 g). ¹HNMR (300 MHz, chloroform-d) δ: 3.82-3.52 (m, 5H), 3.40-3.35 (m, 1H), 3.11-3.09 (m, 1H), 2.75-2.73 (m, 2H).

Synthesis of (S)-(1,4-dioxan-2-yl)methanol: (R)-2-((2-chloroethoxy)methyl)oxirane (400.0 g, 2.94 mol, 1.0 eq) was added to a solution of NaOH (294.0 g, 7.35 mol, 2.5 eq) in water (2900 mL) at 25° C. The reaction mixture was heated on an oil bath for 2 h at 90° C. The resulting solution was cooled to 25° C. and adjusted PH value to 5 by HCl (6.0 M). The mixture was concentrated and the residue was distilled (90-95° C., 0.1 kPa) under vacuum pump to give a colorless oil (S)-(1,4-dioxan-2-yl)methanol (110 g, 31.7%). ¹HNMR: (300 MHz, chloroform-d) δ: 3.85-3.42 (m, 9H), 2.15 (bs, 1H).

Synthesis of (R)-(1,4-dioxan-2-yl)methyl methanesulfonate: A mixture of (S)-(1,4-dioxan-2-yl)methanol (50.0 g, 0.42 mol, 1.0 eq), TEA (63.6 g, 0.63 mol, 1.5 eq) and DCM (500 mL) at ice-bath, MsCl (48.1 g, 0.42 mol, 1.0 eq) was added dropwise. And then, the ice-bath removed and the mixture was stirred at 25° C. for 2 hours. The reaction mixture was washed by water (2×50 mL) and the organic phase was dried over sodium sulfate, and concentrated to give a light brown oil (R)-(1,4-dioxan-2-yl)methyl methanesulfonate (71.0 g, 83%). ¹HNMR (300 MHz, chloroform-d₆) δ 4.23-4.20 (m, 2H), 3.82-3.56 (m, 6H), 3.50-3.40 (m, 1H), 3.02 (m, 3H).

Synthesis of (S)-(1,4-dioxan-2-yl)methanamine: In 1000 mL autoclave, a solution of (R)-(1,4-dioxan-2-yl)methyl methanesulfonate (70.0 g, 0.36 mol, 1.0 eq) in NH₃MeOH (7 M, 500 mL) was stirred at 80° C. for 12 hours, the reaction mixture was cooled to 25° C. and concentrated to give a light brown oil (S)-(1,4-dioxan-2-yl)methanamine (30.0 g, 73%). ¹HNMR (300 MHz, DMSO-d₆) δ 8.27 (bs, 2H), 3.82-3.42 (m, 6H), 3.24-3.20 (m, 1H), 2.98-2.62 (m, 2H).

Synthesis of (S)-4-((1,4-dioxan-2-yl)methylamino)-3-nitrobenzenesulfonamide: A mixture of (S)-(1,4-dioxan-2-yl)methanamine (25.0 g, 0.21 mol, 1.0 eq), 4-fluoro-3-nitrobenzenesulfonamide (46.0 g, 0.21 mol, 1.0 eq) and Cs₂CO₃(137.3 g, 0.42 mol, 2.0 eq) in THE (700 mL) was stirred at 50° C. for 6 hours. LCMS showed material was consumed completely, the reaction mixture was cooled to 25° C. and poured into water (3500 mL). The mixture was filtrated and collected filtrate cake and dried by oven to give a yellow solid (S)-4-((1,4-dioxan-2-yl)methylamino)-3-nitrobenzenesulfonamide (60.0 g, 89.5%). ¹HNMR (300 MHz, DMSO-d₆) δ 8.52-8.47 (m, 2H), 7.86-7.83 (m, 1H), 7.28-7.00 (m, 3H), 3.82-3.29 (m, 9H).

### Example INT_5: Preparation of methyl 2-bromo-4-(4-[[2-(4-chlorophenyl)-4,4-dimethylcyclohex-1-en-1-yl]methyl]piperazin-1-yl)benzoate

Into a 250-mL round-bottom flask, were placed a solution of Example 1-1, 1-[[2-(4-chlorophenyl)-4,4-dimethylcyclohex-1-en-1-yl]methyl]piperazine (15.09 g, 47.32 mmol, 1.0 eq) in DMA (150 mL), DIEA (12.9 g, 99.81 mmol, 2.0 eq), methyl 2-bromo-4-fluorobenzoate (11.6 g, 49.78 mmol, 1.0 eq). The resulting solution was stirred for 12 h at 100° C. The reaction mixture was cooled to 25° C. The reaction was then quenched by the addition of 50 mL of water. The resulting solution was extracted with ethyl acetate (3×100 mL) and the organic layers combined. The resulting mixture was washed with brine (3×100 mL). The mixture was dried over anhydrous sodium sulfate, then filtered and concentrated under vacuum. The residue was applied onto column (silica gel, ethyl acetate/petroleum ether=0:1 to 1:5). This resulted in 7 g (crude) of methyl 2-bromo-4-(4-[[2-(4-chlorophenyl)-4,4-dimethylcyclohex-1-en-1-yl]methyl]piperazin-1-yl)benzoate as yellow oil. LC-MS (ESI, m/z) M+1: 533.

### Example INT_6: Preparation of 2-bromo-4-(4-((2-(4-chlorophenyl)-4,4-dimethylcyclohex-1-enyl)methyl)piperazin-1-yl)-N-(3-nitro-4-((tetrahydro-2H-pyran-4-yl)methylamino)phenylsulfonyl)benzamide

Synthesis of methyl 2-bromo-4-(4-((2-(4-chlorophenyl)-4,4-dimethylcyclohex-1-enyl) methyl)piperazin-1-yl)benzoate: Into a 20000-mL round-bottom flask, were placed 1-((2-(4-chlorophenyl)-4,4-dimethylcyclohex-1-enyl)methyl)piperazine dihydrochloride (600 g, 1.53 mol, 1.0 eq), methyl 2-bromo-4-fluorobenzoate (357 g, 1.53 mol, 1.0 eq), DBU (319 g, 6.12 mol, 4.0 eq) and DMSO (8000 mL). The resulting solution was stirred for 20 h at 70° C. LCMS showed material was completely consumed. The resulting mixture was cooled to 25° C. and poured into water (32 L). The mixture was filtrated, collection of filter cake and the filter cake was washed by water (3×3000 mL) and dried by oven to give product (740 g, 91%) methyl 2-bromo-4-(4-((2-(4-chlorophenyl)-4,4-dimethylcyclohex-1-enyl)methyl)piperazin-1-yl)benzoate as a white solid. ¹HNMR: (300 MHz, DMSO-d₆) δ: 7.73 (d, J=9.0 Hz, 1H), 7.42-7.39 (m, 2H), 7.18-7.12 (m, 3H), 6.97-6.94 (m, 1H), 4.00-3.84 (m, 2H), 3.76 (s, 2H), 3.57 (s, 3H), 3.51-3.33 (m, 4H), 2.79-2.60 (m, 2H), 2.32-2.30 (m, 2H), 2.03-1.97 (m, 2H), 1.47-1.45 (m, 2H), 0.96 (s, 6H).

Synthesis of 2-bromo-4-(4-((2-(4-chlorophenyl)-4,4-dimethylcyclohex-1-enyl) methyl)piperazin-1-yl)benzoic acid: Into a 20000-mL round-bottom flask, were placed methyl 2-bromo-4-(4-((2-(4-chlorophenyl)-4,4-dimethylcyclohex-1-enyl)methyl)piperazin-1-yl)benzoate (730 g, 1.37 mol, 1.0 eq), LiOH (131.5 g, 5.48 mol, 4.0 eq) and MeOH/THF/water (4500 mL/3000 mL/1000 mL). The resulting solution was stirred for 16 h at 70° C. LCMS showed material was completely consumed. The resulting mixture was cooled to 25° C. and concentrated. The residue was diluted with water (5000 mL) and the mixture was adjust pH to 3-5 with HCl (6.0 M), followed by filtrated, collection of filter cake and dried by oven to give product (650 g, 93%) 2-bromo-4-(4-((2-(4-chlorophenyl)-4,4-dimethylcyclohex-1-enyl)methyl)piperazin-1-yl)benzoic acid as a white solid. ¹HNMR: (300 MHz, DMSO-d₆) δ: 10.60 (bs, 1H), 7.73 (d, J=8.4 Hz, 1H), 7.42-7.39 (m, 2H), 7.14-7.11 (m, 3H), 6.95-6.92 (m, 1H), 4.00-3.84 (m, 2H), 3.76 (s, 2H), 3.51-3.33 (m, 4H), 2.79-2.60 (m, 2H), 2.32-2.30 (m, 2H), 2.03-1.97 (m, 2H), 1.47-1.45 (m, 2H), 0.97 (s, 6H).

Synthesis of 2-bromo-4-(4-((2-(4-chlorophenyl)-4,4-dimethylcyclohex-1-enyl) methyl)piperazin-1-yl)-N-(3-nitro-4-((tetrahydro-2H-pyran-4-yl)methylamino)phenylsulfonyl)benzamide: Into a 20000-mL round-bottom flask, were placed 2-bromo-4-(4-((2-(4-chlorophenyl)-4,4-dimethylcyclohex-1-enyl)methyl)piperazin-1-yl)benzoic acid (583 g, 1.13 mol, 1.0 eq), DCM (10 L), 3-nitro-4-[[(oxan-4-yl)methyl]amino]benzene-1-sulfonamide (338 g, 1.07 mol, 0.95 eq), EDCI (326 g, 1.7 mol, 1.5 eq), DMAP (551 g, 4.52 mol, 4.0 eq). The resulting solution was stirred for overnight at 25° C. LCMS showed material was completely consumed. The resulting mixture is followed by dilute hydrochloric acid (1.0 M) (3×1000 mL), saturated sodium bicarbonate (3×1000 mL) and brine (1000 mL), and then the organic phase was dried by Na₂SO₄, filtrated. The filtrate was concentrated to give product (857 g, 93%) as a light brown yellow solid 2-bromo-4-(4-((2-(4-chlorophenyl)-4,4-dimethylcyclohex-1-enyl)methyl)piperazin-1-yl)-N-(3-nitro-4-((tetrahydro-2H-pyran-4-yl)methylamino)phenylsulfonyl)benzamide as a brown yellow solid. LC-MS (ESI, m/z) M+1: 814, 816, 818. ¹HNMR (300 MHz, DMSO-d₆) δ 8.63-8.61 (m, 2H), 7.94-7.92 (m, 1H), 7.37-7.35 (m, 3H), 7.27-7.24 (m, 1H), 7.05-7.02 (m, 3H), 6.86-6.83 (m, 1H), 3.87-3.82 (m, 2H), 3.37-3.23 (m, 8H), 2.92 (s, 2H), 2.50-2.38 (m, 4H), 2.22-2.20 (m, 2H), 2.00-1.97 (m, 2H), 1.64-1.60 (m, 2H), 1.48-1.46 (m, 2H), 1.26-1.20 (m, 2H), 0.97 (s, 6H).

### Example INT_7: Preparation of methyl 2. ((1H-pyrrolo [2,3-b] pyridin-5-yl) oxy)-4-bromobenzoate

A mixture of methyl 4-bromo-2-fluorobenzoate (116.5 g, 0.5 mol), 1H-pyrrolo [2,3-b] pyridin-5-ol (67 g, 0.5 mol) and K₂CO₃ (138 g, 1.0 mol) in DMF (500 mL) was heated at 95° C. for about 16 h. The reaction mixture was cooled to ambient temperature, filtered and the filtrate was diluted with DCM (1 L). The resulting solution was washed with water (2×500 mL) and concentrated. The residue was recrystallized from ethyl acetate (200 mL) and petroleum ether (400 mL), the cake (68 g) was collected as the first batch. The filtrate was concentrated and dissolved in ethyl acetate (500 mL). The solution was washed with water (2×200 mL), concentrated, and slurried with ethyl acetate (25 mL) and petroleum ether (25 mL) at reflux for 1 h, cooled to 95° C., filtered to give the product (38 g) as the second batch. The two batches of product were combined to afford the product (106 g, 61.3%) as a brown solid. LC-MS (ESI, m/z) M+1: 346.9, 348.9.

### Example INT_8: Preparation of 2. (2-cyclopropylphenyl) pyrrolidine

Synthesis of tert-butyl 2. (2-bromophenyl) pyrrolidine-1-carboxylate: A mixture solution of 2-(2-bromophenyl) pyrrolidine (1.13 g, 5 mmol), Boc₂O (2.16 g, 10 mmol), TEA (1.01 g, 10 mmol) and DMAP (cat) in DCM (20 mL) was stirred at 25° C. for 16 hours. Then the mixture solution was concentrated, and the residue was purified by chromatography on silica-gel (petroleum ether/ethyl acetate=5/1) to give the product (1.6 g, 98.1%) as a colorless oil. LC-MS (ESI, m/z) M+1: 270.0.

Synthesis of tert-butyl 2. (2-cyclopropylphenyl) pyrrolidine-1-carboxylate: Under a nitrogen atmosphere, a mixture of tert-butyl 2-(2-bromophenyl) pyrrolidine-1-carboxylate (1.56 g, 4.7 mmol), cyclopropyl boronic acid (1.23 g, 14.3 mmol), Pd(PPh₃)₄ (540 mg, 0.47 mmol) and K₂CO₃ (1.99 g, 14.3 mmol) in 1,4-dioxane/water (9:1, 20 mL) was stirred at 90° C. for 16 hours. Then the reaction mixture was filtered and concentrated, the crude product (1.4 g) was used directly in next step without purification. LC-MS (ESI, m/z) M+1: 232.1.

Synthesis of 2. (2-cyclopropylphenyl) pyrrolidine: A mixture solution of tert-butyl 2-(2-cyclopropylphenyl) pyrrolidine-1-carboxylate (1.4 g) and TFA (5 mL) in DCM (50 mL) was stirred at 25° C. for 16 hours. Then the mixture was concentrated to give a product (1.2 g, crude) as a yellow oil. LC-MS (ESI, m/z) M+1: 188.1.

### Example INT_9: Preparation of 1. (4-bromophenyl)-2-phenylpyrrolidine

To a degassed solution of 2-phenylpyrrolidine (588 mg, 4 mmol), 1-bromo-4-iodobenzene (1.132 g, 16 mmol), BINAP (497 mg, 0.8 mmol) and KOtBu (1.2 g, 12 mmol) in toluene (25 mL) was added Pd₂(dba)₃ (366 mg, 0.4 mmol). Nitrogen was bubbled through the mixture for 5 min, then heated to 90° C. and stirred overnight. After cooled to 25° C., the reaction mixture was washed with water and brine in sequence. The organic layer was dried over anhydrous Na₂SO₄, then filtered, concentrated and purified by column chromatography to give 1-(4-bromophenyl)-2-phenylpyrrolidine (750 mg, 62%) as a colorless oil. LC-MS (ESI, m/z) M+1: 302.0.

### Example INT_10: Preparation of (S)-2. (2-(2-cyclopropylphenyl) pyrrolidin-1-yl)-7-azaspiro[3.5] nonane

Synthesis of tert-butyl (S)-2. (2-bromophenyl) pyrrolidine-1-carboxylate: To a solution of (S)-2-(2-bromophenyl) pyrrolidine (70 g, 311 mmol) in DCM (200 mL) were added Boc₂O (72.6 g, 333 mmol) and DMAP (cat) at 0° C. After addition, the mixture was stirred at 25° C. for 1 hour. Then the mixture solution was washed with saturated aq. NaHCO₃ (3×100 mL), brine. The organic phase was dried with anhydrous NaSO₄, filtered, and concentrated to obtain pale brown solid (95g, crude), which was not further purified for next step. LC-MS (ESI, m/z) M+1: 326.1.

Synthesis of tert-butyl (S)-2. (2-cyclopropylphenyl) pyrrolidine-1-carboxylate: Under a N₂ atmosphere, a mixture of tert-butyl (S)-2-(2-bromophenyl) pyrrolidine-1-carboxylate (88 g, 270 mmol), cyclopropyl boronic acid (68.9 g, 810 mmol), Pd(dqpf)₂Cl₂ (19.7 g, 27 mmol) and K₂CO₃ (150 g, 1.08 mol) in 1,4-dioxane (270 mL) and water (30 mL) was stirred at 90° C. for 16 hours. After the reaction mixture was cooled to 25° C., the mixture was filtered, and the filtrate was concentrated in vacuum. The residue was purified by chromatography column on silica (DCM/CH₃OH=20: 1) to obtain tert-butyl (S)-2-(2-cyclopropylphenyl) pyrrolidine-1-carboxylate as pale yellow oil 70 g (yield: 90%). LC-MS (ESI, m/z) M+1: 288.1.

Synthesis of (S)-2. (2-cyclopropylphenyl) pyrrolidine: To a solution of tert-butyl (S)-2-(2-cyclopropylphenyl) pyrrolidine-1-carboxylate (70g, 244 mmol) in DCM (200 mL) was added HCl solution (200 mL, 4 M in dioxane). After addition, the mixture was stirred for overnight at 25° C. After removal of solvent, the residue was diluted with water (200 mL) and ethyl acetate (100 mL) under stirring. the separated water phase was adjusted to pH=11 and exacted with DCM (2×100 mL). The combined organic phase was dried with anhydrous NaSO₄, filtered, and concentrated to obtain (S)-2-(2-cyclopropylphenyl) pyrrolidine (42 g, crude) as brown oil. LC-MS (ESI, m/z) M+1: 188.2.

Synthesis of tert-butyl (S)-2. (2-(2-cyclopropylphenyl) pyrrolidin-1-yl)-7-azaspiro[3.5] nonane-7-carboxylate: To the mixture of (S)-2-(2-cyclopropylphenyl) pyrrolidine (3.74 g, 20 mmol) and tert-butyl 2-oxo-7-azaspiro[3.5] nonane-7-carboxylate (4.78 g, 20 mmol) in DCM (100 mL) was added NaBH(AcO)₃ (8.48 g, 40 mmol) at 25° C. and stirred for 2 hours. The reaction mixture was quenched with aq. NaHCO₃ solution (200 mL), and then extracted with DCM (2×200 mL). The organic layer was combined, washed with brine and dried over Na₂SO₄. After evaporation in vacuum, the crude product (8.21 g) was obtained as a colorless oil without further purification for next step. LC-MS (ESI, m/z) M+1: 411.0.

Synthesis of (S)-2. (2-(2-cyclopropylphenyl) pyrrolidin-1-yl)-7-azaspiro[3.5] nonane: To a solution of tert-butyl (S)-2-(2-(2-cyclopropylphenyl) pyrrolidin-1-yl)-7-azaspiro[3.5] nonane-7-carboxylate (8.2 g, 20 mmol) in DCM (200 mL) was added TFA (40 mL) at 20° C. and stirred at 20° C. overnight. The mixture was concentrated in vacuum and diluted with DCM (200 mL) and aq. NaOH solution (1 M, 200 mL) was added under stirring. Then the organic layer was collected and dried over Na₂SO₄. After evaporation in vacuum, the crude product (6.2 g) was obtained as a brown oil without further purification for next step. LC-MS (ESI, m/z) M+1: 311.0. ¹HNMR (400 MHz, chloroform-d) δ 7.61 (dd, J=8.0 Hz, J=4.0 Hz, 1H), 7.22-7.11 (m, 2H), 7.00-6.96 (m, 1H), 3.94 (t, J=8.0 Hz, 1H), 3.23-3.03 (m, 2H), 2.71-2.60 (m, 4H), 2.42-2.20 (m, 2H), 2.07-1.55 (m, 10H), 1.41-1.37 (m, 3H), 0.95-0.87 (m, 2H), 0.64-0.53 (m, 2H).

### Example INT_11: Preparation of 2. (2-(2-cyclopropylphenyl)-4-methylpiperazin-1-yl)-7-azaspiro[3.5]nonane

Synthesis of methyl 2-bromo-2. (2-bromophenyl) acetate: To a solution of methyl 2-(2-bromophenyl) acetate (25 g, 109.14 mmol) in CCl₄ (250 mL) was added NBS (21.37 g, 120.65 mmol) and BPO (1.32 g, 5.46 mmol). The mixture was stirred at 85° C. for 5 hours. TLC showed reactant was consumed completely. The mixture was poured into water (200 mL) and extracted with DCM (3×200 mL), washed with brine, dried over Na₂SO₄, filtered and concentrated. The residue was purified by prep-MPLC. Methyl 2-bromo-2-(2-bromophenyl) acetate (20 g) was obtained as a colorless oil. ¹HNMR (400 MHz, chloroform-d) δ 7.79 (dd, J=1.0, 8.0 Hz, 1H), 7.58 (dd, J=1.0, 8.0 Hz, 1H), 7.40-7.34 (m, 1H), 7.21 (dt, J=1.5, 7.7 Hz, 1H), 5.98-5.83 (m, 1H), 3.82 (s, 3H).

Synthesis of 3. (2-bromophenyl) piperazin-2-one: To a solution of methyl 2-bromo-2-(2-bromophenyl) acetate (20 g, 64.94 mmol) in MeOH (200 mL) was added DIEA (12.67 g, 98.04 mmol) and ethane-1,2-diamine (7.86 g, 130.72 mmol). The mixture was stirred at 25° C. for 12 hours. TLC showed reactant was consumed completely. The mixture was diluted with water (200 mL) and extracted with ethyl acetate (3×200 mL), washed with brine, dried over Na₂SO₄, filtered and concentrated to give crude 3-(2-bromophenyl) piperazin-2-one (14 g) as a white solid, which used in next step without further purification.

Synthesis of tert-butyl 2. (2-(2-bromophenyl)-3-oxopiperazin-1-yl)-7-azaspiro[3.5] nonane-7-carboxylate: To a solution of 3-(2-bromophenyl) piperazin-2-one (7 g, 27.44 mmol) and tert-butyl 2-oxo-7-azaspiro[3.5] nonane-7-carboxylate (7.22 g, 30.18 mmol) in DCE (150 mL) was added AcOH (3.3 g, 54.88 mmol) and NaBH(OAc)₃ (11.63 g, 54.88 mmol). The mixture was stirred at 25° C. for 12 hours. TLC showed reactant was consumed completely. The reaction mixture was extracted with aq. Na₂CO₃ (150 mL) and ethyl acetate (3×150 mL), dried over Na₂SO₄, filtered and concentrated. The residue was purified by prep-MPLC. Tert-butyl 2-(2-(2-bromophenyl)-3-oxopiperazin-1-yl)-7-azaspiro[3.5] nonane-7-carboxylate (8 g) was obtained as a white solid.

Synthesis of tert-butyl 2. (2-(2-cyclopropylphenyl)-3-oxopiperazin-1-yl)-7-azaspiro[3.5]nonane-7-carboxylate: To a solution of tert-butyl 2-(2-(2-bromophenyl)-3-oxopiperazin-1-yl)-7-azaspiro[3.5]nonane-7-carboxylate (8 g, 16.72 mmol) and cyclopropylboronic acid (2.15 g, 25.08 mmol) in dioxane (100 mL) and water (10 mL) was added Cs₂CO₃ (16.34 g, 50.16 mmol) and Pd(dqpf)Cl₂ (1.22 g, 1.67 mmol) under N₂ atmosphere. The mixture was stirred at 85° C. for 2 hours. LCMS showed reactant was consumed completely and one main peak with desired MS signal. The reaction mixture was filtered and concentrated. The residue was diluted with water (50 mL)/ethyl acetate (50 mL) and extracted with ethyl acetate (2×50 mL), dried over Na₂SO₄, filtered and concentrated. The residue was purified by prep-MPLC. Tert-butyl 2-(2-(2-cyclopropylphenyl)-3-oxopiperazin-1-yl)-7-azaspiro[3.5] nonane-7-carboxylate (5 g, 68.02% yield) was obtained as a yellow solid. LC-MS (ESI, m/z) M+1: 440.2.

Synthesis of tert-butyl 2. (2-(2-cyclopropylphenyl)-4-methyl-3-oxopiperazin-1-yl)-7-azaspiro[3.5] nonane-7-carboxylate: To a solution of tert-butyl 2-(2-(2-cyclopropylphenyl)-3-oxopiperazin-1-yl)-7-azaspiro[3.5] nonane-7-carboxylate (5 g, 11.37 mmol) in THE (50 mL) was added NaH (0.5 g, 12.51 mmol, 60%) at 0° C. The mixture was stirred at 0° C. for 10 minutes. Then Mel (3.23 g, 22.75 mmol) was added at 0° C. The mixture was stirred at 25° C. for 5 hours. LCMS showed reactant was consumed completely and one main peak with desired MS signal. The reaction mixture was diluted with water (50 mL) and extracted with ethyl acetate (3×50 mL), dried over Na₂SO₄, filtered and concentrated. The residue was purified by prep-MPLC. Tert-butyl 2-(2-(2-cyclopropylphenyl)-4-methyl-3-oxopiperazin-1-yl)-7-azaspiro[3.5] nonane-7-carboxylate (4.7 g, 91.09% yield) was obtained as a white solid. LC-MS (ESI, m/z) M+1: 454.2.

Synthesis of tert-butyl 2. (2-(2-cyclopropylphenyl)-4-methylpiperazin-1-yl)-7-azaspiro[3.5]nonane-7-carboxylate: A mixture of tert-butyl 2-(2-(2-cyclopropylphenyl)-4-methyl-3-oxopiperazin-1-yl)-7-azaspiro[3.5] nonane-7-carboxylate (4.7 g, 10.36 mmol, 1 eq) in BH₃·THF (50 mL) was stirred at 70° C. for 12 hours. LCMS showed reactant was consumed completely and one main peak with desired MS signal. The reaction mixture was quenched by MeOH (50 mL) at 0° C. and stirred at 25° C. for 30 minutes. Then the mixture was concentrated to afford tert-butyl 2-(2-(2-cyclopropylphenyl)-4-methylpiperazin-1-yl)-7-azaspiro[3.5]nonane-7-carboxylate (4.5 g). LC-MS (ESI, m/z) M+1: 440.3.

Synthesis of 2. (2-(2-cyclopropylphenyl)-4-methylpiperazin-1-yl)-7-azaspiro[3.5] nonane: A mixture of tert-butyl 2-(2-(2-cyclopropylphenyl)-4-methylpiperazin-1-yl)-7-azaspiro[3.5] nonane-7-carboxylate (4.5 g, 10.24 mmol) in DCM (25 mL) and TFA (25 mL) was stirred at 25° C. for 1 hours. LCMS showed reactant was consumed completely and one main peak with desired MS signal. The reaction mixture was concentrated under reduced pressure to remove solvent. The residue was purified by prep-HPLC (TFA condition). The desired collection was concentrated and was dilute with water (20 mL) and added aq.Na₂CO₃ to adjust pH to 9. The mixture was extracted with ethyl acetate (3×20 mL), dried over Na₂SO₄, filtered and concentrated under reduced pressure to remove solvent. 2-(2-(2-cyclopropylphenyl)-4-methylpiperazin-1-yl)-7-azaspiro[3.5] (1.2g) was obtained as a white solid. ¹H NMR (400 MHz, chloroform-d) δ 7.48 (s, 1H), 7.22-7.12 (m, 2H), 7.01 (s, 1H), 6.39 (s, 1H), 3.96 (d, J=7.3 Hz, 1H), 3.04-2.96 (m, 1H), 2.95-2.87 (m, 2H), 2.85-2.65 (m, 5H), 2.30 (s, 3H), 2.27 (s, 2H), 2.12 (s, 1H), 1.99 (s, 1H), 1.90-1.81 (m, 1H), 1.75-1.66 (m, 1H), 1.60-1.43 (m, 4H), 1.38-1.28 (m, 1H), 1.11 (d, J=4.4, 7.2, 11.5 Hz, 1H), 0.99-0.91 (m, 2H), 0.70 (s, 1H), 0.63-0.52 (m, 1H). LC-MS (ESI, m/z) M+1: 340.3.

### Example INT_12: Preparation of (11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene and (11S,15R)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene(Method A)

Synthesis of N-[(trans)(3S,4R)-4-hydroxyoxolan-3-yl]-4-methylbenzenesulfonamide: Into a 250 mL round-bottom flask purged and maintained with an inert atmosphere of nitrogen, were placed 3,6-dioxabicyclo[3-1-0]hexane (5.0 g, 58.1 mmol, 1.0 eq), dioxane (100 mL), p-toluenesulfonamide (11.9 g, 69.7 mmol, 1.2 eq), N-benzyl-N,N-diethylethanaminium chloride (TEBAC) (1.3 g, 5.8 mmol, 0.1 eq), K₂CO₃ (0.8 g, 5.8 mmol, 0.1 eq). The resulting solution was stirred for 3 days at 90° C. in an oil bath. The reaction mixture was cooled to 25° C. The solids were filtered out. The resulting mixture was concentrated under vacuum. The crude residue was purified by a flash column (silica gel, ethyl acetate/petroleum ether=2: 1). This resulted in N-[(trans)(3S,4R)-4-hydroxyoxolan-3-yl]-4-methylbenzenesulfonamide (11 g, 25.7%) as white solid. LC-MS (ESI, m/z) M+1: 258.

Synthesis of N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide: Into a 250 mL round-bottom flask purged and maintained with an inert atmosphere of nitrogen, were placed N-[(trans)(3S,4R)-4-hydroxyoxolan-3-yl]-4-methylbenzenesulfonamide (10.5 g, 14.7 mmol, 1.3 eq, 36%), tetrahydrofuran (100 mL). This was followed by the addition of NaH (2.7 g, 67.7 mmol, 6.0 eq, 60%), in portions at 0° C. The resulting solution was stirred for 30 mins at 0° C. To this was added 5-bromo-6-fluoro-1-[[2-(trimethylsilyl)ethoxy]methyl]pyrrolo[2,3-b]pyridine (3.9 g, 11.2 mmol, 1.0 eq) at 0° C. The resulting solution was stirred for 4 hours at 70° C. in an oil bath. The reaction mixture was cooled to 25° C. The reaction was then quenched by the addition of aqueous NH₄Cl (500 mL). The resulting solution was extracted with ethyl acetate (3×300 mL) and the organic layers combined. The resulting mixture was washed with brine (1×1500 mL). The mixture was dried over anhydrous Na₂SO₄. After filtration, the filtrate was concentrated under reduced pressure. The crude residue was purified by a flash column (silica gel, ethyl acetate/petroleum ether=1: 2) to afford N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide (3 g, 45.7%) as light yellow oil. ¹HNMR (300 MHz, Chloroform-d) δ 8.04 (s, 1H), 7.78-7.61 (m, 2H), 7.25-7.10 (m, 3H), 6.43 (d, J=3.6 Hz, 1H), 5.67 (d, J=10.8 Hz, 1H), 5.60 (d, J=5.7 Hz, 1H), 5.49 (d, J=10.8 Hz, 1H), 5.40 (dt, J=6.1, 3.1 Hz, 1H), 4.27 (dd, J=10.5, 5.9 Hz, 1H), 4.21-4.03 (m, 1H), 4.03-3.86 (m, 2H), 3.70-3.52 (m, 3H), 2.32 (s, 3H), 0.93 (ddt, J=10.6, 5.5, 2.6 Hz, 2H), −0.02 (s, 9H).

Synthesis of (trans)(11R,15S)-10-(4-methylbenzenesulfonyl)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene: Into a 250 mL round-bottom flask purged and maintained with an inert atmosphere of nitrogen, were placed N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide (3.0 g, 5.1 mmol, 1.0 eq), N,N-dimethylformamide (50 mL), 1,10-phenanthroline (743 mg, 4.1 mmol, 0.8 eq), CuI (785 mg, 4.1 mmol, 0.8 eq), K₂CO₃ (2.1 g, 15.5 mmol, 3.0 eq). The resulting solution was stirred for 2 days at 120° C. in an oil bath. The reaction mixture was cooled to 25° C. The resulting solution was diluted with water (500 mL). The resulting solution was extracted with ethyl acetate (3×200 mL) and the organic layers combined. The resulting mixture was washed with brine (1×1000 mL). The mixture was dried over anhydrous Na₂SO₄. After filtration, the filtrate was concentrated under reduced pressure. The crude residue was purified by a flash column (silica gel, ethyl acetate/petroleum ether=1: 2) to afford (trans)(11R,15S)-10-(4-methylbenzenesulfonyl)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene (2 g, 82.2%) as yellow oil. LC-MS (ESI, m/z) M+1: 502.

Synthesis of (11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene and (11S,15R)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene: Into a 100 mL round-bottom flask, were placed Mg (2.0 g, 83.9 mmol, 19.8 eq), MeOH (30 mL), (trans)(11R,15S)-10-(4-methylbenzenesulfonyl)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene (2.5 g, 4.2 mmol, 1.0 eq, 85%). The resulting solution was stirred for 2 hours at 60° C. in an oil bath. The reaction mixture was cooled to 25° C. The resulting solution was diluted with 300/300 mL of NaHCO₃ and CH₂Cl₂. The solids were filtered out and the organic was separated. The mixture was dried over anhydrous Na₂SO₄. After filtration, the filtrate was concentrated under reduced pressure. The crude residue was purified by a flash column (silica gel, ethyl acetate/petroleum ether=1: 3). The crude product was purified by Chiral-Prep-HPLC with the following conditions: Mobile phase: A: n-Hexane (0.1% DEA) B: EtOH; Flow rate: 20 mL/min; Column: DAICEL CHIRALPAK IA, 250*20 mm, 5 um; Gradient:12% B in 20 min; 220 nm. This resulted in (11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (350 mg, 23.7%) as yellow oil. and (11S,15R)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene (400 mg, 27.1%) as yellow oil. Peak 1: LC. MS (ESI, m/z) M+1: 348. Peak 1, T_{R}=0.99 min in CHIRAL-HPLC, Column: CHIRALPAK IA-3, 4.6*50 mm, 3 μm. mobile phase A: n-Hexane (0.1% DEA); mobile phase B: Ethanol, Conc. of Phase B: 50.0%, Column Temp: 25° C. ¹HNMR (300 MHz, Chloroform-d) δ 7.39 (s, 1H), 7.21 (d, J=3.6 Hz, 1H), 6.37 (d, J=3.6 Hz, 1H), 5.57 (s, 2H), 4.60 (dt, J=10.1, 7.6 Hz, 1H), 4.39-4.22 (m, 2H), 3.94 (dd, J=9.9, 7.9 Hz, 1H), 3.86-3.69 (m, 2H), 3.64-3.45 (m, 2H), 0.91 (dd, J=8.8, 7.5 Hz, 2H), −0.04 (s, 9H). Peak 2: LC-MS (ESI, m/z) M+1: 348. Peak 2, T_{R}=1.34 min in CHIRAL-HPLC, Column: CHIRALPAK IA-3, 4.6*50 mm, 3 μm. mobile phase A: n-Hexane (0.1% DEA); mobile phase B: Ethanol, Conc. of Phase B: 50.0%, Column Temp: 25° C. ¹HNMR (300 MHz, Chloroform-d) δ 7.39 (s, 1H), 7.21 (d, J=3.6 Hz, 1H), 6.37 (d, J=3.6 Hz, 1H), 5.57 (s, 2H), 4.60 (dt, J=10.1, 7.6 Hz, 1H), 4.39-4.22 (m, 2H), 3.94 (dd, J=9.9, 7.9 Hz, 1H), 3.86-3.69 (m, 2H), 3.64-3.45 (m, 2H), 0.91 (dd, J=8.8, 7.5 Hz, 2H), 0.04 (s, 9H).

### Example INT_13: Preparation of (11R,15S)-4-{[2-(trim ethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (Method B)

Synthesis of (3S,4R)-4-{[(1R)-1-phenylethyl]amino}oxolan-3-ol: Into a 1000 mL round-bottom flask were added 3,6-dioxabicyclo[3-1-0]hexane (56.0 g, 650.0 mmol, 1.0 eq), water (300 mL) and D-α-methylbenzylamine (75.0 g, 619.0 mmol, 1.0 eq). The resulting mixture was stirred for overnight at 100° C. The mixture was allowed to cool down to 70° C. 2-propanol (50 mL) was added and the mixture was allowed to slowly cool to 25° C. The precipitated solids were collected by filtration and washed with water (1×100 mL). The resulting solid was dried under infrared light. This resulted in (3S,4R)-4-{[(1R)-1-phenylethyl]amino}oxolan-3-ol (43 g, 33.5%) as a white solid. ¹HNMR (300 MHz, DMSO-d₆) δ 7.37-7.26 (m, 4H), 7.25-7.16 (m, 1H), 4.82 (d, J=4.2 Hz, 1H), 4.04 (tt, J=4.2, 1.9 Hz, 1H), 3.82 (dd, J=9.0, 4.5 Hz, 2H), 3.67 (dd, J=8.6, 5.8 Hz, 1H), 3.44 (dd, J=9.0, 2.0 Hz, 1H), 3.28 (dd, J=8.6, 3.4 Hz, 1H), 2.80 (s, 1H), 2.12 (s, 1H), 1.24 (d, J=6.6 Hz, 3H).

Synthesis of (3R,4S)-4-[(5-bromo-1-{[2-(trimethylsilyl)ethoxy]methyl}pyrrolo[2,3-b]pyridin-6-yl)oxy]-N-[(1R)-1-phenylethyl]oxolan-3-amine: Into a 1000 mL 3-necked round-bottom flask were added (3S,4R)-4-{[(1R)-1-phenylethyl]amino}oxolan-3-ol (43.0 g, 207.5 mmol, 1.0 eq), THE (500 mL). To the above mixture was added NaH (24.9 g, 622.4 mmol, 3.0 eq, 60%) in portions at 0° C. The resulting mixture was stirred for additional 0.5 hour at 0° C. To the above mixture was added 5-bromo-6-fluoro-1-{[2-(trimethylsilyl)ethoxy]methyl}pyrrolo[2,3-b]pyridine (86.0 g, 248.9 mmol, 1.2 eq) dropwise at 0° C. The resulting mixture was stirred for additional overnight at 70° C. The mixture was cooled to 25° C. The resulting mixture was diluted with water (400 mL). The aqueous layer was extracted with Ethyl acetate (2×400 mL) and the organic layer was washed with brine (2×400 mL), the combined organic layer was dried over anhydrous Na₂SO₄. After filtration, the filtrate was concentrated under reduced pressure. The crude residue was purified by a flash column (silica gel, ethyl acetate/petroleum ether=1: 2) to afford (3R,4S)-4-[(5-bromo-1-{[2-(trimethylsilyl)ethoxy]methyl}pyrrolo[2,3-b]pyridin-6-yl)oxy]-N-[(1R)-1-phenylethyl]oxolan-3-amine (75 g, 67.9%) as a white solid. LC-MS (ESI, m/z) M+1: 532.

Synthesis of (11R,15S)-10-[(1R)-1-phenylethyl]-4-{[2-(trimethylsilyl)ethoxy]methyl})-13,16-dioxa-2,4,10-triazatetracyclo[7.7.0.0\hat{ }{3,7}.0\hat{ }{11,15}]hexadeca-1(9),2,5,7-tetraene: Into a 2 L round-bottom flask were added (3R,4S)-4-[(5-bromo-1-{[2-(trimethylsilyl)ethoxy]methyl}pyrrolo[2,3-b]pyridin-6-yl)oxy]-N-[(1R)-1-phenylethyl]oxolan-3-amine (75.0 g, 140.8 mmol, 1.0 eq), dioxane (750 mL), Cs₂CO₃ (91.8 g, 281.7 mmol, 2 eq) and Pd(dtbpf)Cl₂ (9.2 g, 14.1 mmol, 0.1 eq). The resulting mixture was stirred for overnight at 80° C. under N₂ protection. The mixture was cooled to 25° C. The resulting mixture was concentrated under reduced pressure. The crude residue was purified by a flash column (silica gel, ethyl acetate/petroleum ether=1: 2) to afford (11R,15S)-10-[(1R)-1-phenylethyl]-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (37 g, 58.2%) as a white solid. ¹HNMR (300 MHz, DMSO-d₆) δ 7.46 (d, J=7.6 Hz, 2H), 7.37 (t, J=7.5 Hz, 2H), 7.33-7.23 (m, 2H), 7.18 (s, 1H), 6.18 (d, J=3.5 Hz, 1H), 5.39 (s, 2H), 4.82-4.61 (m, 2H), 4.30 (t, J=7.0 Hz, 1H), 4.20 (t, J=7.5 Hz, 1H), 3.74 (dt, J=10.4, 7.2 Hz, 2H), 3.46 (t, J=8.0 Hz, 2H), 1.51 (d, J=6.9 Hz, 4H), 0.93-0.74 (m, 2H), −0.09 (s, 9H).

Synthesis of (11R,15S)-4-{[2-(trim ethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene: Into a 1000 mL round-bottom flask were added (11R,15S)-10-[(1R)-1-phenylethyl]-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (37.0 g, 81.9 mmol, 1.0 eq), EtOH (400 mL) and Pd(OH)₂/C (3.4 g, 24.6 mmol, 0.3 eq). The resulting mixture was stirred for overnight at 25° C. under hydrogen (3 atm) atmosphere. The resulting mixture was filtered; the filter cake was washed with EtOH (2×200 mL). The filtrate was concentrated under reduced pressure. The crude residue was purified by a flash column (silica gel, ethyl acetate/petroleum ether=1: 3) to afford (11R,15S)-4-{[2-(trim ethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (25 g, 87.8%) as a white solid. ¹HNMR (300 MHz, Chloroform-d) δ 7.39 (s, 1H), 7.21 (d, J=3.6 Hz, 1H), 6.37 (d, J=3.6 Hz, 1H), 5.57 (s, 2H), 4.60 (dt, J=10.1, 7.6 Hz, 1H), 4.39-4.22 (m, 2H), 3.94 (dd, J=9.9, 7.9 Hz, 1H), 3.86-3.69 (m, 2H), 3.64-3.45 (m, 2H), 0.91 (dd, J=8.8, 7.5 Hz, 2H), −0.04 (s, 9H).

### Example INT_14: Preparation of (2S)-2-(2-bromophenyl)pyrrolidine

Synthesis of (S)—N-[(2-bromophenyl)methylidene]-2-methylpropane-2-sulfinamide: Into a 3000-mL round-bottom flask, were placed 2-bromo-benzaldehyde (120.0 g, 648.6 mmol, 1.0 eq), THE (2000 mL), (S)-2-methylpropane-2-sulfinamide (79.0 g, 651.8 mmol, 1.0 eq), Ti(OEt)₄ (297.0 g, 1302.0 mmol, 2.0 eq). The resulting solution was stirred overnight at 55° C. in an oil bath. The reaction mixture was cooled to 25° C. The resulting mixture was concentrated under vacuum. The resulting solution was diluted with water and Ethyl acetate (2000/2000 mL). The solids were filtered out. The organic layer was separated from the filtration. The mixture was dried over anhydrous sodium sulfate. After filtration, the filtrate was concentrated under reduced pressure. This resulted in (S)—N-[(2-bromophenyl)methylidene]-2-methylpropane-2-sulfinamide (181 g, 96.8%) as yellow oil. LC-MS (ESI, m/z) M+1: 288. ¹HNMR (300 MHz, Chloroform-d) δ 8.81 (d, J=1.7 Hz, 1H), 8.03 (dt, J=6.6, 1.7 Hz, 1H), 7.84-7.74 (m, 1H), 7.54 (dq, J=5.6, 3.7, 2.4 Hz, 2H), 1.19 (d, J=1.7 Hz, 9H).

Synthesis of (S)—N-[(1S)-1-(2-bromophenyl)-3-(1,3-dioxan-2-yl)propyl]-2-methylpropane-2-sulfinamide: Into a 1000-mL 3-necked round-bottom flask purged and maintained with an inert atmosphere of nitrogen, were placed Mg (37.1 g, 1527.7 mmol, 4.0 eq), I₂ (9.8 g, 38.7 mmol, 0.1 eq), THE (300 mL), the resulting solution was warmed to 65° C., followed by the addition of 2-(2-bromoethyl)-1,3-dioxane (30.0 g, 153.8 mmol, 0.4 eq) in THE (50 mL) at 65° C. After addition completed, the mixture was cooled to 25° C., another 2-(2-bromoethyl)-1,3-dioxane (270.0 g, 1384.6 mmol, 3.6 eq) in THE (150 mL) was added. Another 3000-mL 3-necked round-bottom flask purged and maintained with an inert atmosphere of nitrogen, were placed a solution of (S)—N-[(2-bromophenyl)methylidene]-2-methylpropane-2-sulfinamide (111.0 g, 385.1 mmol, 1.0 eq) in CH₂Cl₂ (800 mL). The above solution was added dropwise at −40° C. The resulting solution was stirred for 1 hour at −40° C. in a liquid nitrogen bath. The reaction was then quenched by the addition of aqueous NH₄Cl (2000 mL). The resulting solution was extracted with dichloromethane (3×1000 mL) and the organic layers combined. The resulting mixture was washed with brine (1×3000 mL). The mixture was dried over anhydrous sodium sulfate. After filtration, the filtrate was concentrated under reduced pressure. This resulted in (S)—N-[(1S)-1-(2-bromophenyl)-3-(1,3-dioxan-2-yl)propyl]-2-methylpropane-2-sulfinamide (150 g, 96.3%) as yellow oil. LC-MS (ESI, m/z) M+1: 404.

Synthesis of (2S)-2-(2-bromophenyl)pyrrolidine: Into a 2000-mL round-bottom flask, were placed (S)—N-[(1S)-1-(2-bromophenyl)-3-(1,3-dioxan-2-yl)propyl]-2-methylpropane-2-sulfinamide (150.0 g, 370.9 mmol, 1.0 eq), TFA (360 mL), water (90 mL), the resulting solution was stirred for 1 hour at 25° C. TFA (1350 mL), triethylsilane (129.4 g, 1112.8 mmol, 3.0 eq) was added. The resulting solution was stirred overnight at 25° C. The resulting mixture was concentrated. The resulting solution was diluted with water (2000 mL). The resulting solution was extracted with MTBE (2×500 mL) and the organic layers combined. The resulting mixture was washed with 2.0 M HCl (1×1000 mL) and the aqueous layers combined. The pH value of the aqueous was adjusted to 13 with NaOH (4 mol/L). The resulting solution was extracted with dichloromethane (3×500 mL) and the organic layers combined and dried over anhydrous sodium sulfate. After filtration, the filtrate was concentrated under reduced pressure. This resulted in (2S)-2-(2-bromophenyl)pyrrolidine (40 g, 47.7%) as yellow oil. LC-MS (ESI, m/z) M+1: 226. ¹HNMR (300 MHz, DMSO-d₆) δ 7.67 (dd, J=7.8, 1.8 Hz, 1H), 7.53 (dd, J=7.9, 1.3 Hz, 1H), 7.34 (td, J=7.5, 1.3 Hz, 1H), 7.13 (td, J=7.6, 1.8 Hz, 1H), 4.33 (t, J=7.4 Hz, 1H), 3.10-2.87 (m, 2H), 2.35-2.18 (m, 1H), 1.80-1.64 (m, 2H), 1.40-1.20 (m, 1H).

### Example INT_15: Preparation of tert-butyl 2-[(2S)-2-(2-bromophenyl)pyrrolidin-1-yl]-7-azaspiro[3.5]nonane-7-carboxylate

Synthesis of tert-butyl 2-[(2S)-2-(2-bromophenyl)pyrrolidin-1-yl]-7-azaspiro[3.5]nonane-7-carboxylate: Into a 250-mL round-bottom flask, were placed (2S)-2-(2-bromophenyl)pyrrolidine (6.2 g, 27.3 mmol, 1.0 eq), CH₂Cl₂ (100 mL), tert-butyl 2-oxo-7-azaspiro[3.5]nonane-7-carboxylate (6.6 g, 27.4 mmol, 1.0 eq), NaBH(OAc)₃ (11.6 g, 54.9 mmol, 2.0 eq). The resulting solution was stirred for 2 hours at 25° C. The resulting mixture was concentrated under vacuum. The crude residue was purified by a flash column (silica gel, ethyl acetate/petroleum ether=1:1) to afford tert-butyl 2-[(2S)-2-(2-bromophenyl)pyrrolidin-1-yl]-7-azaspiro[3.5]nonane-7-carboxylate (8 g, 64.3%) as yellow oil. LC-MS (ESI, m/z) M+1: 449.

### Example INT_16: Preparation of (2S)-2-(2-isopropylphenyl)pyrrolidine hydrochloride

Synthesis of tert-butyl (2S)-2-(2-bromophenyl)pyrrolidine-1-carboxylate: Into a 250-mL round-bottom flask, was placed (2S)-2-(2-bromophenyl)pyrrolidine (17.0 g, 75.2 mmol, 1.0 eq), CH₂Cl₂ (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1:15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311/313. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-[2-(prop-1-en-2-yl)phenyl]pyrrolidine-1-carboxylate: Into a 500 mL round-bottom flask were added tert-butyl (2S)-2-(2-bromophenyl)pyrrolidine-1-carboxylate (10.0 g, 30.6 mmol, 1.0 eq) and 4,4,5,5-tetramethyl-2-(prop-1-en-2-yl)-1,3,2-dioxaborolane (15.5 g, 92.2 mmol, 3.0 eq), dioxane (180 mL), water (20 mL), K₂CO₃ (12.7 g, 91.9 mmol, 3.0 eq), Pd(dppf)Cl₂·CH₂Cl₂ (2.5 g, 3.1 mmol, 0.1 eq). The resulting mixture was stirred for overnight at 80° C. under nitrogen atmosphere. The mixture was allowed to cool down to 25° C. The resulting mixture was concentrated under vacuum. The crude residue was purified by a flash column (silica gel, ethyl acetate/petroleum ether=1:10) to afford tert-butyl (2S)-2-[2-(prop-1-en-2-yl)phenyl]pyrrolidine-1-carboxylate (8 g, 90.8%) as a yellow oil. LC-MS (ESI, m/z) M-t-Bu+CH₃CN: 273.

Synthesis of tert-butyl (2S)-2-(2-isopropylphenyl)pyrrolidine-1-carboxylate: Into a 250 mL pressure tank reactor (10 atm) were added tert-butyl (2S)-2-[2-(prop-1-en-2-yl)phenyl]pyrrolidine-1-carboxylate (8.0 g, 27.8 mmol, 1.0 eq), MeOH (100 mL), Pd/C (0.8 g). The resulting mixture was stirred for overnight at 50° C. under hydrogen atmosphere. The resulting mixture was filtered, the filter cake was washed with MeOH (100 mL). The filtrate was concentrated under reduced pressure. The crude residue was purified by a flash column (silica gel, ethyl acetate/petroleum ether=1:10) to afford tert-butyl (2S)-2-(2-isopropylphenyl)pyrrolidine-1-carboxylate (6 g, 80.6%) as a colorless oil. LC-MS (ESI, m/z) M-t-Bu+CH₃CN: 275.

Synthesis of (2S)-2-(2-isopropylphenyl)pyrrolidine hydrochloride: A solution of tert-butyl (2S)-2-(2-isopropylphenyl)pyrrolidine-1-carboxylate (6.5 g, 22.5 mmol, 1.0 eq) in HCl (gas) in 1,4-dioxane (4 M, 70 mL) was stirred for 1 h at 25° C. The resulting mixture was concentrated under vacuum. This resulted in (2S)-2-(2-isopropylphenyl)pyrrolidine hydrochloride (5 g, 98.6%) as a light yellow oil. LC-MS (ESI, m/z) M-HCl+CH₃CN: 231. ¹HNMR (300 MHz, Methanol-d₄) δ 7.57-7.28 (m, 4H), 5.06 (t, J=7.8 Hz, 1H), 3.52 (q, J=6.9 Hz, 2H), 3.41-3.26 (m, 1H), 2.57-2.42 (m, 1H), 2.42-2.12 (m, 3H), 1.35 (d, J=6.8 Hz, 3H), 1.27 (d, J=6.8 Hz, 3H).

### Example INT_17: Preparation of (2S)-2-(2-methylphenyl)pyrrolidine

Synthesis of (S)-2-methyl-N-[(2-methylphenyl)methylidene]propane-2-sulfinamide: Into a 500 mL 3-necked round-bottom flask were added 2-methylbenzaldehyde (20.0 g, 166.4 mmol, 1.0 eq) and (S)-2-methylpropane-2-sulfinamide (24.2 g, 199.7 mmol, 1.2 eq), tetrahydrofuran (200 mL), Ti(OEt)₄ (56.9 g, 249.6 mmol, 1.5 eq) at 25° C. The resulting mixture was stirred for overnight at 55° C. The reaction was quenched by the addition of water (300 mL) at 25° C. The resulting mixture was extracted with Ethyl acetate (3×200 mL). The combined organic layers were washed with brine (1×300 mL), dried over anhydrous Na₂SO₄. After filtration, the filtrate was concentrated under reduced pressure. This crude resulted in (S)-2-methyl-N-[(2-methylphenyl)methylidene]propane-2-sulfinamide (30 g, 80.7%) as light yellow oil. LC-MS (ESI, m/z) M+1: 224. ¹HNMR (300 MHz, DMSO-d₆) δ 8.73 (s, 1H), 7.88 (dd, J=7.7, 1.6 Hz, 1H), 7.45 (td, J=7.4, 1.5 Hz, 1H), 7.43-7.25 (m, 2H), 2.54 (s, 3H), 1.17 (s, 9H).

Synthesis of (S)—N-[(1S)-3-(1,3-dioxan-2-yl)-1-(2-methylphenyl)propyl]-2-methylpropane-2-sulfinamide: Into a 1000 mL 3-necked round-bottom flask were added magnesium (5.4 g, 223.8 mmol, 2.0 eq) and iodine (60 mg, 0.2 mmol, 0.002 eq), tetrahydrofuran (20 mL) at 25° C. 2-(2-bromoethyl)-1,3-dioxane (43.6 g, 223.8 mmol, 2.0 eq) in tetrahydrofuran (220 mL) was added dropwise at 25° C. under nitrogen atmosphere. The resulting mixture was stirred for 30 mins at 25° C. To the above mixture was added (S)-2-methyl-N-[(2-methylphenyl)methylidene]propane-2-sulfinamide (25 g, 111.942 mmol, 1.00 eq) in CH₂Cl₂(100 mL) dropwise 10 mins at −40° C. The resulting mixture was stirred for additional 5 hours at −40° C. The reaction was quenched with sat.NH₄Cl (aq.) at 25° C. The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=10:01) to afford (S)—N-[(1S)-3-(1,3-dioxan-2-yl)-1-(2-methylphenyl)propyl]-2-methylpropane-2-sulfinamide (35 g, 92.1%) as light yellow oil. LC-MS (ESI, m/z) M+1: 340.

Synthesis of (2S)-2-(2-methylphenyl)pyrrolidine: Into a 500 mL 3-necked round-bottom flask were added (S)—N-[(1S)-3-(1,3-dioxan-2-yl)-1-(2-methylphenyl)propyl]-2-methylpropane-2-sulfinamide (35.0 g, 103.1 mmol, 1.0 eq) and Trifluoroacetic acid (20 mL), water (18 mL) at 25° C. The resulting mixture was stirred for 1 hour at 25° C. To the above mixture was added Trifluoroacetic acid (210 mL), TESiH (40.2 g, 309.3 mmol, 3.0 eq). The resulting mixture was stirred for additional 20 hours at 25° C. The resulting mixture was concentrated under reduced pressure. The resulting mixture was diluted with water (300 mL). The aqueous layer was extracted with methyl tert-butyl ether (300 mL). The aqueous layer was basified to pH to 13 with 1 M NaOH. The resulting mixture was extracted with CH₂Cl₂ (2×300 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=10:1-1:1) to afford (2S)-2-(2-methylphenyl)pyrrolidine (15 g, crude)(ee=87%). The crude residue was dissolved in ethanol ethyl acetate=35:65 (300 mL). To the above mixture was added dibenzoyl-L-taratric acid at 25° C. The resulting mixture was stirred for additional 10 mins at 90° C. The mixture was allowed to cool down to 25° C. The precipitated solids were collected by filtration and washed with Ethyl acetate (20 mL). The crude residue was dissolved in water (100 mL). The mixture was acidified to pH 10 with NaOH (1.0 M). The resulting mixture was extracted with CH₂Cl₂ (3×200 mL). The combined organic layers dried over anhydrous Na₂SO₄. After filtration, the filtrate was concentrated under reduced pressure. This crude resulted in (2S)-2-(2-methylphenyl)pyrrolidine (6.5 g, 39.1%) (ee=98.6%) as yellow oil. LC-MS (ESI, m/z) M+1:162. ¹HNMR (300 MHz, DMSO-d₆) δ 7.52 (dd, J=7.2, 1.5 Hz, 1H), 7.19-7.01 (m, 3H), 4.19 (t, J=7.5 Hz, 1H), 3.04 (dd, J=9.7, 7.1, 5.4 Hz, 1H), 2.89 (dt, J=9.7, 7.5 Hz, 1H), 2.56 (s, 1H), 2.29 (s, 3H), 2.14 (dtd, J=12.1, 7.7, 5.8 Hz, 1H), 1.74 (dd, J=12.3, 9.8 Hz, 2H), 1.41-1.13 (m, 1H).

### Example INT_18 Preparation of methyl 2-bromo-4-{2-oxo-7-azaspiro[3.5]nonan-7-yl}benzoate

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2 HCl in Ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1:2) to afford methyl 2-bromo-4-{2-oxo-7-azaspiro[3.5]nonan-7-yl}benzoate (6.8 g, 46.5%) as light yellow solid. LC-MS (ES, m/z) M+1: 352/354. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

### Example INT_19 Preparation of 2-bromo-N-{3-nitro-4-[(oxan-4-ylmethyl)amino]benzenesulfonyl}-4-{2-oxo-7-azaspiro[3.5]nonan-7-yl}benzamide

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4.0 eq) at 25° C. The resulting mixture was stirred for 2 hours at 40° C. The mixture was allowed to cool down to 25° C. The resulting mixture was diluted with water (20 mL). The mixture was acidified to pH=6 with HCl (1.0 M). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ES, m/z) M+1: 338/340. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ES, m/z) M+1: 635/637. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

### Example INT_20 Preparation of N-((3-nitro-4-(((tetrahydro-2H-pyran-4-yl)methyl)amino)phenyl)sulfonyl)-4-(2-oxo-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide

Synthesis of N-((3-nitro-4-(((tetrahydro-2H-pyran-4-yl)methyl)amino)phenyl)sulfonyl)-4-(2-oxo-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10:1) to afford N-((3-nitro-4-(((tetrahydro-2H-pyran-4-yl)methyl)amino)phenyl)sulfonyl)-4-(2-oxo-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (400 mg, 47.0%) as yellow solid. LC-MS (ES, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dp, J=16.8, 5.4, 4.9 Hz, 3H), 0.91-0.76 (m, 3H), −0.06 (s, 9H).

### Example INT_21 Preparation of methyl 2-bromo-4-{2-oxo-7-azaspiro[3.5]nonan-7-yl}benzoate

Synthesis of (2S)-2-(2-isopropoxyphenyl)pyrrolidine: Into a 100 mL round-bottom flask were added (2S)-2-(2-bromophenyl)pyrrolidine (2.0 g, 8.8 mmol, 1.0 eq), i-PrOH (20 mL), sodium propan-2-olate (2.2 g, 26.7 mmol, 3.0 eq), CuBr (631 mg, 4.4 mmol, 0.5 eq). The resulting mixture was stirred for overnight at 95° C. The mixture was allowed to cool down to 25° C. The resulting mixture was concentrated under reduced pressure. The crude residue was purified by a flash column (silica gel, dichloromethane/methanol=10:1) to afford (2S)-2-(2-isopropoxyphenyl)pyrrolidine (770 mg, 42.4%) as a yellow oil. LC-MS (ESI, m/z) M+1: 206.

### Example INT_22 Preparation of S)-3-(o-tolyl)morpholine and (R)-3-(o-tolyl)morpholine

Synthesis of 2-amino-2-(o-tolyl)ethan-1-ol: Into a 1000 mL round-bottom flask purged and maintained with an inert atmosphere of nitrogen. To a stirred solution of 2-amino-2-(o-tolyl)acetic acid (10.0 g, 60.5 mmol, 1.0 eq) were added tetrahydrofuran (100 mL) dropwise at 0° C. under N₂ atmosphere. To the above mixture was added borane-THF (200 mL) dropwise over 1 hour at 0° C. The resulting mixture was stirred for additional 16 hours at 25° C. The reaction was quenched by the addition of HCl (1.0 M, 300 mL) at 0° C. The resulting mixture was concentrated under reduced pressure. The mixture was basified to pH=12 with NaOH (4.0 M). The aqueous layer was extracted with ethyl acetate (3×200 ml), The combined organic layers and dried over anhydrous Na₂SO₄. After filtration, the filtrate was concentrated under reduced pressure. The crude product was used in the next step directly without further purification. This resulted in 2-amino-2-(o-tolyl)ethan-1-ol (4.4 g, 36.5%) as a white solid. LC-MS (ES, m/z) M+1: 152.

Synthesis of 2-chloro-N-(2-hydroxy-1-(o-tolyl)ethyl)acetamide: Into a 250 mL round-bottom flask purged and maintained with an inert atmosphere of nitrogen. To a stirred solution of 2-amino-2-(o-tolyl)ethan-1-ol (2.2 g, 14.6 mmol, 1.0 eq) and Triethylamine (2.4 g, 23.3 mmol, 1.5 eq) in CH₂Cl₂ (22 mL) was added 2-chloroacetyl chloride (2.0 g, 17.5 mmol, 1.2 eq) in portions at 0° C. The resulting mixture was stirred for 3 hours at 0° C. The mixture was concentrated under reduced pressure. The crude residue was purified by a flash column (silica gel, ethyl acetate/petroleum ether=1:1) to afford 2-chloro-N-(2-hydroxy-1-(o-tolyl)ethyl)acetamide (1.5 g, 45.9%) as a white oil. LC-MS (ES, m/z) M+1: 228.

Synthesis of 5-(o-tolyl)morpholin-3-one: Into a 250 mL 3-necked round-bottom flask were added 2-chloro-N-(2-hydroxy-1-(o-tolyl)ethyl)acetamide (1.5 g, 6.4 mmol, 1.0 eq), THE (30 mL). To the above mixture was added NaH (0.6 g, 12.8 mmol, 2.0 eq, 60%) in portions at 0° C. The resulting mixture was stirred for additional 0.5 hour at 0° C. The resulting mixture was stirred for additional overnight at 25° C. The resulting mixture was quenched by water (40 mL). The aqueous layer was extracted with ethyl acetate (2×40 mL) and the organic layer was washed with brine (2×40 mL), the combined organic layer was dried over anhydrous Na₂SO₄. After filtration, the filtrate was concentrated under reduced pressure. The crude residue was purified by a flash column (silica gel, ethyl acetate/petroleum ether=2:1) to afford 5-(o-tolyl)morpholin-3-one (0.7 g, 67.9%) as a white solid. LC. MS (ES, m/z) M+1: 192.

Synthesis of 3-(o-tolyl)morpholine: Into a 40 ml glass bottle flask purged and maintained with an inert atmosphere of nitrogen. To a stirred solution 5-(o-tolyl)morpholin-3-one (600 mg, 3.1 mmol, 1.0 eq) in THE (6 mL) was added LiAlH₄ (490 mg, 3.1 mmol, 4.0 eq) in portions at 0° C. under N₂ atmosphere. The resulting mixture was stirred for 16 hours at 25° C. under N₂ atmosphere. The reaction was quenched with water (0.49 mL), NaOH (15% aq, 0.49 mL) and water (1.5 mL) at 0° C. After filtration of reaction solution, the filter cake was washed with MeOH (3×10 mL). The resulting mixture was concentrated under reduced pressure. The mixture was combined and dried over anhydrous sodium sulfate and concentrated under vacuum. The residue was purified by Prep-TLC (dichloromethane/methanol=12:1) to afford 3-(o-tolyl)morpholine (400 mg, 77.3%) as a white oil. The crude product was used in the next step directly without further purification. LC-MS (ES, m/z) M+1: 178.

Synthesis of S)-3-(o-tolyl)morpholine and (R)-3-(o-tolyl)morpholine: The 400 mg of 3-(o-tolyl)morpholine was purified by Chiral-Prep-HPLC with the following conditions: Column: CHIRAL ART Cellulose-SB, 3*25 cm, 5 μm; mobile phase A: Hex—HPLC; mobile phase B: EtOH—HPLC; Flow rate: 35 mL/min; Gradient: 10% B to 0% B in 0 min; Wave Length: 220/254 nm. This resulted in 160 mg of S)-3-(o-tolyl)morpholine as a white oil. This resulted in 150 mg of (R)-3-(o-tolyl)morpholine as a white oil. LC-MS (ES, m/z) M+1: 178. S)-3-(o-tolyl)morpholine, T_{R}=2.323 min in CHIRAL-HPLC, Column: CHIRAL ART Cellulose-SB, 3*25 cm, 5 μm. mobile phase A: n-Hexane; mobile phase B: Ethanol, Pump Mode: Low pressure gradient, Conc. of Pump B: 10.0%, Oven Temperature: 25° C. LC-MS (ES, m/z) M+1: 178. (R)-3-(o-tolyl)morpholine, T_{R}=2.577 min in CHIRAL-HPLC, Column: CHIRAL ART Cellulose-SB, 3*25 cm, 5 μm. mobile phase A: n-Hexane; mobile phase B: Ethanol, Pump Mode: Low pressure gradient, Conc. of Pump B: 10.0%, Oven Temperature: 25° C.

### Example INT_23 Preparation of 2-bromo-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of 2-bromo-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide: Into a 250 mL round-bottom flask were added (2S)-2-(2-isopropoxyphenyl)pyrrolidine (1.0 g, 4.8 mmol, 1.0 eq) and 2-bromo-N-{3-nitro-4-[(oxan-4-ylmethyl)amino]benzenesulfonyl}-4-{2-oxo-7-azaspiro[3.5]nonan-7-yl}benzamide (3.0 g, 4.7 mmol, 1.0 eq), CH₂Cl₂ (50 mL), NaBH(OAc)₃ (2.0 g, 9.4 mmol, 2.0 eq). The resulting mixture was stirred for overnight at 40° C. The mixture was allowed to cool down to 25° C. The resulting mixture was concentrated under vacuum. The crude residue was purified by a flash column (silica gel, dichloromethane/methanol=10:1) to afford 2-bromo-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide (2800 mg, 58.9%) as a yellow solid. LC-MS (ES, m/z) M+1: 824/826. ¹HNMR (400 MHz, DMSO-d₆) δ 9.99 (s, 1H), 8.56-8.48 (m, 2H), 7.90 (dd, J=9.1, 2.3 Hz, 1H), 7.62 (d, J=7.1 Hz, 1H), 7.39 (t, J=7.8 Hz, 2H), 7.20 (d, J=9.2 Hz, 1H), 7.09 (d, J=8.4 Hz, 1H), 7.03-6.94 (m, 2H), 6.83 (dd, J=8.8, 2.4 Hz, 1H), 4.73 (dt, J=13.5, 6.7 Hz, 2H), 3.89-3.81 (m, 2H), 3.54 (s, 2H), 3.35 (d, J=6.6 Hz, 2H), 3.27 (td, J=11.7, 2.0 Hz, 2H), 3.21-2.99 (m, 5H), 2.32 (d, J=9.1 Hz, 1H), 2.13 (s, 5H), 1.92 (ddp, J=11.4, 7.9, 3.9 Hz, 1H), 1.80 (s, 1H), 1.67-1.58 (m, 2H), 1.53 (s, 4H), 1.48 (d, J=5.8 Hz, 1H), 1.38-1.22 (m, 8H).

### Example INT_24 Preparation of 3-nitro-4-([[(1r,4r)-4-hydroxy-4-methylcyclohexyl]methyl]amino)benzenesulfonamide and 3-nitro-4-([[(1s,4s)-4-hydroxy-4-methylcyclohexyl]methyl]amino)benzenesulfonamide

Synthesis of 8-methyl-1,4-dioxaspiro[4.5]decan-8-ol: Into a 2-L 3-necked round-bottom flask purged and maintained with an inert atmosphere of nitrogen, was placed 1,4-dioxaspiro[4.5]decan-8-one (100.0 g, 640.3 mmol, 1.0 eq), THE (500 mL). This was followed by the addition of CH₃MgBr (256 mL, 3.0 M, 1.2 eq) in portions at 0° C. The resulting solution was stirred for 4 hours at 0° C. The reaction was then quenched by the addition of aqueous NH₄Cl. The resulting mixture was concentrated. The residue was dissolved in dichloromethane (500 mL). The resulting mixture was washed with water (3×50 mL). The mixture was dried over anhydrous sodium sulfate. After filtration, the filtrate was concentrated under reduced pressure. This resulted in 8-methyl-1,4-dioxaspiro[4.5]decan-8-ol (110 g, 99.7%) as white oil. ¹HNMR (300 MHz, Chloroform-d) δ: 3.98-3.93 (m, 4H), 2.07-1.52 (m, 8H), 1.27 (s, 3H).

Synthesis of 4-hydroxy-4-methylcyclohexan-1-one: Into a 1-L round-bottom flask, was placed 8-methyl-1,4-dioxaspiro[4.5]decan-8-ol (110.0 g, 639.5 mmol, 1.0 eq), HCl (0.05 M, 500 mL). The resulting solution was stirred for 4 hours at 70° C. in an oil bath. The mixture was allowed to cool down to 25° C. The resulting mixture was extracted with Ethyl acetate (3×300 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1:2) to afford 4-hydroxy-4-methylcyclohexan-1-one (40 g, 48.9%) as yellow oil. ¹HNMR (300 MHz, Chloroform-d) δ 2.73 (ddd, J=15.3, 12.9, 6.3 Hz, 2H), 2.33-2.18 (m, 2H), 2.06-1.93 (m, 2H), 1.93-1.78 (m, 2H), 1.38 (s, 3H).

Synthesis of 1-methyl-4-(nitromethyl)cyclohex-3-en-1-ol: Into a 500-mL round-bottom flask, was placed 4-hydroxy-4-methylcyclohexan-1-one (20.0 g, 156.0 mmol, 1.0 eq), CH₃NO₂ (100 mL), methyl[2-(methylamino)ethyl]amine (1.4 g, 15.6 mmol, 0.1 eq). The resulting solution was stirred for 6 hours at 100° C. in an oil bath. The mixture was allowed to cool down to 25° C. The resulting mixture was concentrated. The crude residue was purified by a flash column (silica gel, ethyl acetate/petroleum ether=1:3) to afford 1-methyl-4-(nitromethyl)cyclohex-3-en-1-ol (21 g, 78.6%) as brown oil. ¹HNMR (300 MHz, Chloroform-d) δ 5.87 (t, J=2.7 Hz, 1H), 4.87 (s, 2H), 2.41-2.11 (m, 4H), 1.85-1.62 (m, 2H), 1.54 (d, J=16.0 Hz, 1H), 1.31 (s, 3H).

Synthesis of 4-(aminomethyl)-1-methylcyclohexan-1-ol: Into a 100-mL pressure tank reactor (20 atm), was placed 1-methyl-4-(nitromethyl)cyclohex-3-en-1-ol (5.0 g, 29.2 mmol, 1.0 eq), i-PrOH (50 mL), Pt/C (1.1 g, 5.8 mmol). To the above, H₂ (gas) was introduced in. The resulting solution was stirred for 20 hours at 30° C. in an oil bath. The solids were filtered out. The resulting mixture was concentrated. This resulted in 4-(aminomethyl)-1-methylcyclohexan-1-ol (2.5 g, crude) as black oil.

Synthesis of 3-nitro-4-([[(1r,4r)-4-hydroxy-4-methylcyclohexyl]methyl]amino)benzenesulfonamide and 3-nitro-4-([[(1s,4s)-4-hydroxy-4-methylcyclohexyl]methyl]amino)benzenesulfonamide: Into a 100-mL round-bottom flask, was placed 4-(aminomethyl)-1-methylcyclohexan-1-ol (2.5 g, crude), 4-fluoro-3-nitrobenzenesulfonamide (3.8 g, 17.4 mmol, 1.0 eq), THE (30 mL), TEA (5.3 g, 52.4 mmol, 3.0 eq). The resulting solution was stirred for 14 hours at 25° C. The resulting mixture was concentrated. The residue was applied onto a C18 gel column with (water:CH₃CN=100:0 to 100:30). This resulted in 1 g of 3-nitro-4-([[(1r,4r)-4-hydroxy-4-methylcyclohexyl]methyl]amino)benzenesulfonamide as a yellow solid. ¹HNMR (400 MHz, DMSO-d₆) δ 8.51 (d, J=6.4 Hz, 1H), 8.47 (s, 1H), 7.85 (d, J=9.2 Hz, 1H), 7.36 (s, 2H), 7.27 (dd, J=9.2, 2.4 Hz, 1H), 4.26 (d, J=2.8 Hz, 1H), 3.34 (s, 2H), 1.69 (d, J=13.6 Hz, 3H), 1.55 (d, J=12.8 Hz, 2H), 1.34 (t, J=12.8 Hz, 2H), 1.16 (d, J=12.8 Hz, 1H), 1.10-1.06 (m, 4H). This resulted in 500 mg of 3-nitro-4-([[(1s,4s)-4-hydroxy-4-methylcyclohexyl]methyl]amino)benzenesulfonamide as a yellow solid. ¹HNMR (400 MHz, DMSO-d₆) δ 8.54 (d, J=6.4 Hz, 1H), 8.47 (s, 1H), 7.84 (d, J=9.2 Hz, 1H), 7.34 (s, 2H), 7.26 (d, J=9.2 Hz, 1H), 3.96 (s, 1H), 3.32 (d, J=6.4 Hz, 2H), 3.20-3.11 (m, 1H), 1.75-1.50 (m, 2H), 1.48-1.30 (m, 4H), 1.23 (td, J=13.2, 3.6 Hz, 3H), 1.08 (s, 3H).

### Example INT_25 Preparation of 4-((((1s,4s)-4-methoxy-4-methylcyclohexyl)methyl)amino)-3-nitrobenzenesulfonamide and 4-((((1r,4r)-4-methoxy-4-methylcyclohexyl)methyl)amino)-3-nitrobenzenesulfonamide

Synthesis of methyl 8-methoxy-1,4-dioxaspiro[4.5]decane-8-carboxylate: Into a 250 mL round-bottom, were placed 1,4-dioxaspiro[4.5]decan-8-one (179.1 g, 1.15 mol, 1.0 eq), CHBr₃ (1300 mL). This was followed by the addition of a solution of KOH (516.2 g, 9.2 mol, 8 eq) in MeOH (1800 mL) at −5° C. The resulting solution was stirred for 3 hours at 0° C. and gradually warmed to 25° C. The reaction mixture was concentrated, and then diluted with water (1000 mL) and extracted with Ethyl acetate (600 mL×3). The combined organics were dried over Na₂SO₄, filtered and concentrated in vacuo. The crude residue was purified by a flash column (silica gel, ethyl acetate/petroleum ether=20:1). This resulted in methyl 8-methoxy-1,4-dioxaspiro[4.5]decane-8-carboxylate (83.8 g, 31.7%) as a colorless oil.

Synthesis of (8-methoxy-1,4-dioxaspiro[4.5]decan-8-yl)methanol: Into a 250 mL round-bottom flask purged and maintained with an inert atmosphere of nitrogen, were placed methyl 8-methoxy-1,4-dioxaspiro[4.5]decane-8-carboxylate (83.77 g, 0.36 mol, 1.0 eq), tetrahydrofuran (400 mL). This was followed by the addition of LiAlH₄ (27.6 g, 0.72 mol, 2.0 eq) in portions at 0° C. The resulting solution was stirred for 30 mins at 0° C. The reaction was then quenched by water (84 mL), aqueous NaOH (15%, 84 mL) and water (250 mL) in this order. The resulting solution was filtered, and the filtrate was concentrated in vacuo. This resulted in (8-methoxy-1,4-dioxaspiro[4.5]decan-8-yl)methanol (82.7 g, crude) as a colorless oil, the crude product was used directly to the next step without further purification.

Synthesis of 8-(iodomethyl)-8-methoxy-1,4-dioxaspiro[4.5]decane: Into a 250 mL round-bottom flask purged and maintained with an inert atmosphere of nitrogen were placed (8-methoxy-1,4-dioxaspiro[4.5]decan-8-yl)methanol (82.7 g, 0.41 mol, 1.0 eq), toluene (2500 mL), imidazole (100.2 g, 1.48 mol, 3.6 eq), PPh₃ (376.4 g, 1.435 mol, 3.5 eq). This was followed by the addition of 12 (27.6 g, 0.41 mol, 1.8 eq) in portions at 0° C. The resulting solution was stirred for 2 hours at 90° C. in an oil bath. The reaction mixture was cooled to 25° C. and quenched with aqueous Na₂S₂O₃. The organic phase was collected and the aqueous phase was extracted with ethyl acetate (3×500 mL) and the organic layers were combined. The mixture was dried over anhydrous Na₂SO₄. After filtration, the filtrate was concentrated under reduced pressure. The crude residue was purified by a flash column (silica gel, ethyl acetate/petroleum ether=1:20) to afford 8-(iodomethyl)-8-methoxy-1,4-dioxaspiro[4.5]decane (65.6 g, 51.4%) as colorless oil.

Synthesis of 8-methoxy-8-methyl-1,4-dioxaspiro[4.5]decane: Into a 100 mL round-bottom flask, were placed AIBN (6.9 g, 42 mmol, 0.2 eq), toluene (650 mL), 8-(iodomethyl)-8-methoxy-1,4-dioxaspiro[4.5]decane (65.62 g, 0.21 mol, 1.0 eq) and Bu₃SnH (73 g, 0.25 mol, 1.2 eq). The resulting solution was stirred for 2 hours at 90° C. in an oil bath. The reaction mixture was quenched with aqueous CsF. The resulting solution was diluted with water (500 mL) and extracted with Ethyl acetate (3×300 mL). The combined organics were dried over anhydrous Na₂SO₄. After filtration, the filtrate was concentrated under reduced pressure. The crude residue was purified by a flash column (silica gel, ethyl acetate/petroleum ether=1:20) to afford 8-methoxy-8-methyl-1,4-dioxaspiro[4.5]decane (20.3 g, 51.9%) as colorless oil. ¹HNMR (500 MHz, chloroform-d) δ 3.93-3.91 (m, 4H), 3.16 (s, 3H), 1.81-1.77 (m, 4H), 1.55-1.49 (m, 4H), 1.12 (s, 3H).

Synthesis of 4-methoxy-4-methylcyclohexan-1-one: Into a 100 mL round-bottom flask, were placed 8-methoxy-8-methyl-1,4-dioxaspiro[4.5]decane (20.3 g, 0.21 mol, 1.0 eq) and acetone (200 mL). This was followed by the addition of HCl (2.0 M, 100 mL) at 25° C. The resulting solution was stirred for 48 hours at 25° C. The reaction mixture was quenched with saturated aqueous NaHCO₃ (200 mL). The resulting solution was diluted with water (100 mL) and extracted with Ethyl acetate (3×100 mL). The combined organics were dried over anhydrous Na₂SO₄. After filtration, the filtrate was concentrated under reduced pressure. The resulted in 4-methoxy-4-methylcyclohexan-1-one (18.99 g, crude) as colorless oil.

Synthesis of 4-methoxy-4-methylcyclohexane-1-carbonitrile: Into a 250 mL round-bottom flask purged and maintained with an inert atmosphere of nitrogen, were placed 4-methoxy-4-methylcyclohexan-1-one (4.5 g, 31.64 mmol, 1.0 eq), DME (60 mL), EtOH (1.5 mL), TosMic (6.17 g, 31.64 mmol, 1.0 eq). This was followed by the addition of t-BuOK (7.11 g, 63.28 mmol, 2.0 eq) in portions at 0° C. The resulting solution was stirred for 2 hours at 50° C. in an oil bath. The reaction mixture was cooled to 25° C., and then diluted with MTBE (200 mL). The suspension was filtered and the filtrate was concentrated in vacuo. The crude residue was purified by a flash column (silica gel, ethyl acetate/petroleum ether=1:20) to afford 4-methoxy-4-methylcyclohexane-1-carbonitrile (2.3 g, 47.5%) as colorless oil.

Synthesis of (4-methoxy-4-methylcyclohexyl)methanamine: Into a 250 mL round-bottom flask purged and maintained with an inert atmosphere of nitrogen, was placed 4-methoxy-4-methylcyclohexane-1-carbonitrile (1 g, 6.53 mmol, 1.0 eq), tetrahydrofuran (20 mL). This was followed by the addition of LiAlH₄ (0.97 g, 26.12 mmol, 4.0 eq) in portions at 0° C. The resulting solution was stirred for 2 hours at 25° C. The reaction was then quenched by water (1 mL), aqueous NaOH (15%, 1 mL) and water (3 mL) in this order. The resulting solution was filtered, the filter cake was washed with THE (3×10 mL) and the filtrate was concentrated in vacuo. This resulted in (4-methoxy-4-methylcyclohexyl)methanamine (1 g, a mixture of two diastereoisomers) as a colorless oil, the crude product was used directly to the next step without further purification.

Synthesis of 4-((((1r,4r)-4-methoxy-4-methylcyclohexyl)methyl)amino)-3-nitrobenzenesulfonamide and 4-((((1s,4s)-4-methoxy-4-methylcyclohexyl)methyl)amino)-3-nitrobenzenesulfonamide: Into a 250 mL round-bottom flask purged and maintained with an inert atmosphere of nitrogen, was placed (4-methoxy-4-methylcyclohexyl)methanamine (1 g, 6.36 mmol, a mixture of two diastereoisomers), 4-chloro-3-nitrobenzenesulfonamide (1.81 g, 0.21 mol, 1.2 eq) and DIPEA (3.33 mL, 3 eq). The resulting solution was stirred for 2 hours at 80° C. in an oil bath. The resulting solution was concentrated in vacuo. The crude residue was purified by a flash column (silica gel, DCM/MeOH=100:1) to afford 4-((((1r,4r)-4-methoxy-4-methylcyclohexyl)methyl)amino)-3-nitrobenzenesulfonamide (0.36 g, 15.8%). ¹HNMR (500 MHz, DMSO-d₆) δ 8.53 (t, J=5.5 Hz, 1H), 8.46 (d, J=2.0 Hz, 1H), 8.11 (dd, J=9.5 Hz and 2.0 Hz, 1H), 7.30 (s, 2H), 7.25 (d, J=9.5 Hz, 1H), 3.31-3.29 (m, 2H), 3.05 (s, 3H), 1.78-1.76 (m, 2H), 1.62-1.57 (m, 1H), 1.50-1.48 (m, 2H), 1.25-1.19 (m, 4H), 1.04 (s, 3H). 4-((((1s,4s)-4-methoxy-4-methylcyclohexyl)methyl)amino)-3-nitrobenzenesulfonamide (0.12 g, 5.3%) as yellow solid. ¹HNMR (500 MHz, DMSO-d₆) δ 8.51 (t, J=6.0 Hz, 1H), 8.47 (d, J=2.0 Hz, 1H), 8.12 (dd, J=9.0 Hz and 2.0 Hz, 1H), 7.30 (s, 2H), 7.26 (d, J=9.5 Hz, 1H), 3.36 (t, J=6.0 Hz, 2H), 3.09 (s, 3H), 1.71-1.69 (m, 3H), 1.63-1.61 (m, 2H), 1.40-1.34 (m, 2H), 1.21-1.16 (m, 2H), 1.11 (s, 3H).

### Example INT_26 Preparation of (3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene and (3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene

Synthesis of 4,4-dimethoxyoxan-3-ol: Into a 500 mL round-bottom flask, were placed KOH (10.7 g, 189.8 mmol, 1.9 eq), methanol (200 mL). After that, to the above mixture was added tetrahydro-pyran-4-one (10.0 g, 99.9 mmol, 1.0 eq) in portions over 30 min at 0° C., followed by the addition a solution of I₂ (20.3 g, 79.9 mmol, 0.8 eq) in methanol (400 mL) dropwise over 2 hours at 0° C. The reaction mixture was stirred for 3 hours at 25° C. under nitrogen atmosphere. The resulting mixture was concentrated under vacuum. The residue was dissolved in toluene (100 mL). The solids were filtered off and the filtrate was concentrated under vacuum to give 4,4-dimethoxyoxan-3-ol as a yellow oil (10.0 g, 61.7%).

Synthesis of 3-(benzyloxy)-4,4-dimethoxyoxane: To a stirred solution of 4,4-dimethoxyoxan-3-ol (10.0 g, 61.7 mmol, 1.0 eq) in tetrahydrofuran (200 mL) was added NaH (1.5 g, 61.7 mmol, 1.0 eq) in portions at 0° C. under nitrogen atmosphere. The reaction mixture was stirred for 30 min at 25° C. under nitrogen atmosphere. To the above mixture were added BnBr (10.6 g, 61.7 mmol, 1.0 eq) and tetrabutylammonium iodide (1.1 g, 3.1 mmol, 0.05 eq). The reaction mixture was stirred for 20 hours at 25° C. The resulting mixture was quenched by the addition of aqueous NH₄Cl (100 mL) and extracted with Et₂O (2×100 mL). The combined organic layer was washed with brine (200 mL), dried over anhydrous NaSO₄. After filtration, the filtrate was concentrated under vacuum. The crude residue was applied onto a silica gel column and eluted with ethyl acetate/petroleum ether=1:1 to give 3-(benzyloxy)-4,4-dimethoxyoxane as a colorless oil (8.0 g, 51.4%). ¹HNMR (300 MHz, DMSO-d₆) δ 7.47-7.30 (m, 5H), 4.65 (d, J=11.7 Hz, 1H), 4.50 (d, J=11.7 Hz, 1H), 4.02-3.88 (m, 1H), 3.72-3.64 (m, 1H), 3.50-3.40 (m, 2H), 3.41-3.32 (m, 1H), 3.13 (s, 3H), 3.10 (s, 3H), 1.93-1.56 (m, 2H).

Synthesis of 3-(benzyloxy)oxan-4-one: Into a 500 mL round-bottom flask, were placed 3-(benzyloxy)-4,4-dimethoxyoxane (30.0 g, 118.9 mmol, 1.0 eq), triethylamine (30 mL), water (30 mL) and chloroform (120 mL). The reaction mixture was stirred for 3 hours at 50° C. The mixture was neutralized to pH=7 with saturated aqueous NaHCO₃ and extracted with Et₂O (3×200 mL). The combined organic layers were washed with brine (200 mL), dried over anhydrous Na₂SO₄. After filtration, the filtrate was concentrated under vacuum to give 3-(benzyloxy)oxan-4-one as a colorless oil (21.0 g, 85.6%). ¹HNMR (400 MHz, DMSO-d₆) δ 7.50-7.25 (m, 5H), 4.73 (d, J=11.8 Hz, 1H), 4.51 (d, J=11.8 Hz, 1H), 4.21-4.10 (m, 2H), 4.06 (dddd, J=11.0, 6.8, 2.8, 1.2 Hz, 1H), 3.63 (td, J=11.1, 3.2 Hz, 1H), 3.50-3.38 (m, 1H), 2.71-2.62 (m, 1H), 2.38 (dt, J=14.0, 3.0 Hz, 1H).

Synthesis of 3-(benzyloxy)oxane-4-carbonitrile: Into a 1000 mL 3-necked round-bottom flask, were placed 3-(benzyloxy)oxan-4-one (21.0 g, 101.8 mmol, 1 eq), ethylene glycol dimethyl ether (400 mL), 1-((isocyanomethyl)sulfonyl)-4-methylbenzene (45.7 g, 234.2 mmol, 2.3 eq), tert-Butanol (17.4 g, 234.2 mmol, 2.3 eq). After that, to the above mixture was added t-BuOK (40.0 g, 356.4 mmol, 3.5 eq) in portions over 30 min at 0° C. The reaction mixture was stirred for additional 16 hours at 25° C. The reaction was quenched by the addition of water (100 mL) and extracted with EtOAc (2×200 mL). The combined organic layers were washed with brine (200 mL), dried over anhydrous Na₂SO₄.

After filtration, the filtrate was concentrated under vacuum. The crude residue was applied onto a silica gel column and eluted with ethyl acetate/petroleum ether=1:3 to give 3-(benzyloxy)oxane-4-carbonitrile as a colorless oil (5.0 g, 22.6%). GC-MS (ESI, m/z) M+1: 217.

Synthesis of 1-[3-(benzyloxy)oxan-4-yl]methanamine: Into a 50 mL round-bottom flask, were placed 3-(benzyloxy)oxane-4-carbonitrile (1.2 g, 5.5 mmol, 1.0 eq), methanol (20 mL), Raney Ni (50 mg, 0.6 mmol, 0.1 eq). The reaction mixture was stirred for 5 hours at 25° C. under hydrogen atmosphere. The solids were filtered out and the filter cake was washed with methanol (2×10 mL). The filtrate was concentrated under vacuum to give 1-[3-(benzyloxy)oxan-4-yl]methanamine as a colorless oil (1.0 g, 81.8%).

Synthesis of N-{[3-(benzyloxy)oxan-4-yl]methyl}-4-methylbenzenesulfonamide (trans): Into a 50 mL 3-necked round-bottom flask, were placed 1-[3-(benzyloxy)oxan-4-yl]methanamine (1.1 g, 4.9 mmol, 1.0 eq), dichloromethane (20 mL), triethylamine (1.0 g, 9.9 mmol, 2.0 eq). After that, to the above mixture was added 4-methylbenzenesulfonyl chloride (1.1 g, 6.0 mmol, 1.2 eq) in portions at 0° C. The reaction mixture was stirred for 4 hours at 25° C. The resulting mixture was quenched by the addition of water (10 mL) and extracted with CH₂Cl₂ (2×20 mL). The combined organic layer was washed with brine (20 mL), dried over anhydrous Na₂SO₄. After filtration, the filtrate was concentrated under vacuum. The crude residue was applied onto a silica gel column and eluted with ethyl acetate/petroleum ether=1:1 to give N-{[3-(benzyloxy)oxan-4-yl]methyl}-4-methylbenzenesulfonamide (trans) as a white solid (1.1 g, 58.9%). LC-MS (ESI, m/z) M+1: 376.

Synthesis of N-[(3-hydroxyoxan-4-yl)methyl]-4-methylbenzenesulfonamide (trans): Into a 50 mL pressure tank reactor, were placed N-{[3-(benzyloxy)oxan-4-yl]methyl}-4-methylbenzenesulfonamide (1.0 g, 2.7 mmol, 1.0 eq), methanol (20 mL), Pd/C (10%, 300 mg). The mixture was hydrogenated at 60° C. under 30 atm of hydrogen pressure for overnight, filtered through a Celite pad and concentrated under vacuum. The crude residue was applied onto a silica gel column and eluted with ethyl acetate/petroleum ether=1:2 to give N-[(3-hydroxyoxan-4-yl)methyl]-4-methylbenzenesulfonamide (trans) as a colorless oil (570 mg, 75.0%). ¹HNMR (300 MHz, Chloroform-d) δ 7.82-7.73 (m, 2H), 7.39-7.30 (m, 2H), 5.32 (s, 1H), 3.92 (td, J=10.6, 4.7 Hz, 2H), 3.54 (td, J=9.8, 4.9 Hz, 1H), 3.32 (td, J=11.8, 2.2 Hz, 1H), 3.17 (dd, J=13.0, 4.2 Hz, 1H), 3.07 (dd, J=10.9, 10.0 Hz, 1H), 2.95 (dd, J=13.0, 6.4 Hz, 1H), 2.46 (s, 3H), 1.64 (td, J=14.4, 12.7, 4.6 Hz, 2H), 1.49-1.21 (m, 2H).

Synthesis of N-({3-[(5-bromo-1-{[2-(trimethylsilyl)ethoxy]methyl}pyrrolo[2,3-b]pyridin-6-yl)oxy]oxan-4-yl}methyl)-4-methylbenzenesulfonamide (trans): Into a 250 mL round-bottom flask, were placed N-[(3-hydroxyoxan-4-yl)methyl]-4-methylbenzenesulfonamide (trans) (570 mg, 2.0 mmol, 1.0 eq), tetrahydrofuran (20 mL). After that, to the above mixture was added NaH (240 mg, 6.0 mmol, 3.0 eq, 60%) in portions at 0° C. The reaction mixture was stirred for additional 30 min at 0° C., followed by the addition of 5-bromo-6-fluoro-1-{[2-(trimethylsilyl)ethoxy]methyl}pyrrolo[2,3-b]pyridine (690 mg, 2.0 mmol, 1.0 eq) dropwise at 0° C. The resulting mixture was stirred overnight at 60° C. The resulting mixture was quenched by the addition of aqueous NH₄Cl and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give N-({3-[(5-bromo-1-{[2-(trimethylsilyl)ethoxy]methyl}pyrrolo[2,3-b]pyridin-6-yl)oxy]oxan-4-yl}methyl)-4-methylbenzenesulfonamide (trans) as a light yellow oil (460 mg, 37.7%). ¹HNMR (400 MHz, Chloroform-d) δ 8.05 (s, 1H), 7.66-7.58 (m, 2H), 7.20 (d, J=3.6 Hz, 1H), 7.17-7.06 (m, 2H), 6.46-6.37 (m, 1H), 5.55 (q, J=10.8 Hz, 2H), 5.44 (s, 1H), 4.86 (td, J=9.9, 4.7 Hz, 1H), 4.39 (dd, J=10.7, 4.7 Hz, 1H), 4.03-3.94 (m, 1H), 3.53 (td, J=8.5, 7.2 Hz, 2H), 3.46-3.38 (m, 1H), 3.23 (dd, J=10.7, 9.6 Hz, 1H), 3.05 (s, 2H), 2.34 (s, 3H), 2.15-2.03 (m, 1H), 1.86-1.77 (m, 1H), 1.74-1.59 (m, 1H), 0.90 (ddd, J=9.2, 7.1, 1.1 Hz, 2H), −0.05 (s, 9H).

Synthesis of 10-(4-methylbenzenesulfonyl)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene (trans): Into a 100 mL round-bottom flask, were placed N-({3-[(5-bromo-1-{[2-(trimethylsilyl)ethoxy]methyl}pyrrolo[2,3-b]pyridin-6-yl)oxy]oxan-4-yl}methyl)-4-methylbenzenesulfonamide (trans) (350 mg, 0.6 mmol, 1.0 eq), Dimethyl sulfoxide (15 mL), K₂CO₃ (238 mg, 1.7 mmol, 3.0 eq), CuI (55 mg, 0.3 mmol, 0.5 eq), picolinic acid (35 mg, 0.3 mmol, 0.5 eq). The reaction mixture was stirred overnight at 135° C. The resulting mixture was quenched by the addition of water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=3:2 to give 10-(4-methylbenzenesulfonyl)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene (trans) as a light yellow oil (230 mg, 75.7%). ¹HNMR (300 MHz, Chloroform-d) δ 8.15 (s, 1H), 7.49-7.40 (m, 2H), 7.35 (d, J=3.6 Hz, 1H), 7.20 (d, J=8.0 Hz, 2H), 6.56 (d, J=3.6 Hz, 1H), 5.69 (d, J=10.5 Hz, 1H), 5.51 (d, J=10.6 Hz, 1H), 4.49 (d, J=15.0 Hz, 1H), 4.19 (d, J=10.0 Hz, 1H), 3.90 (dd, J=11.3, 4.5 Hz, 1H), 3.56 (t, J=8.2 Hz, 2H), 3.38-3.25 (m, 2H), 3.04 (t, J=10.4 Hz, 1H), 2.92 (t, J=13.3 Hz, 1H), 2.40 (s, 3H), 2.01 (s, 2H), 1.67 (d, J=13.4 Hz, 1H), 0.92 (td, J=7.8, 4.4 Hz, 2H), −0.03 (s, 9H).

Synthesis of (3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene and (3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene: Into a 40 mL vial purged and maintained with an inert atmosphere of nitrogen, was placed Na (60 mg, 2.6 mmol, 6.0 eq), Naphthalene (334 mg, 2.6 mmol, 6.0 eq), ethylene glycol dimethyl ether (10 mL). The mixture was stirred for 40 min at 25° C. until the formation of Na/naphthalene was complete. At the same time, another 100 mL 3-necked round-bottom flask purged and maintained with an inert atmosphere of nitrogen, were placed 10-(4-methylbenzenesulfonyl)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene (230 mg, 0.4 mmol, 1.0 eq), tetrahydrofuran (10 mL). This was followed by the addition of the above solution at −78° C. The reaction mixture was stirred for 3 hours at 25° C. The resulting mixture was then quenched by the addition of aqueous NH₄Cl (300 mL) and extracted with EtOAc (3×100 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=3:2. The crude product was purified by Prep-SFC using the following conditions. Finally, (3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene was obtained as a brown solid (53 mg, 32.5%) and (3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene was obtained as a brown solid (72 mg, 44.2%). 5A, T_{R}=3.8 min in CHIRAL-SFC, Column: CHIRAL ART Cellulose-SC, 3*25 cm, 5 um; Mobile Phase A: CO2, Mobile Phase B: MeOH; Flow rate: 80 mL/min; Gradient: 30% B, Wave Length: 220 nm. 5B, T_{R}=5.8 min in CHIRAL-SFC, Column: CHIRAL ART Cellulose-SC, 3*25 cm, 5 um; Mobile Phase A: CO2, Mobile Phase B: MeOH; Flow rate: 80 mL/min; Gradient: 30% B, Wave Length: 220 nm. LC-MS (ESI, m/z) M+1: 376 (peak 1). ¹HNMR (300 MHz, Chloroform-d) δ 7.41 (s, 1H), 7.23 (d, J=3.5 Hz, 1H), 6.36 (d, J=3.5 Hz, 1H), 5.67 (d, J=10.7 Hz, 1H), 5.48 (d, J=10.7 Hz, 1H), 4.41 (dd, J=10.0, 4.1 Hz, 1H), 3.95 (dd, J=11.4, 4.6 Hz, 1H), 3.60-3.56 (m, 1H), 3.56-3.53 (m, 1H), 3.53-3.49 (m, 2H), 3.47 (d, J=1.9 Hz, 1H), 3.31 (dd, J=12.5, 3.5 Hz, 1H), 2.67 (dd, J=12.4, 10.0 Hz, 1H), 2.14 (s, 1H), 1.73-1.61 (m, 1H), 1.42 (qd, J=12.6, 4.7 Hz, 1H), 0.90 (ddd, J=9.2, 7.0, 6.0 Hz, 2H), −0.05 (s, 9H).

### Example INT_27 Preparation of (3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene and (3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene

Synthesis of methyl 4-oxooxane-3-carboxylate: Into a 3 L 4-necked round-bottom flask, were placed tetrahydro-pyran-4-one (100.0 g, 998.8 mmol, 1.0 eq), dimethyl carbonate (225.0 g, 2497.8 mmol, 2.5 eq), tetrahydrofuran (1000 mL). After that, to the above stirring solution was added NaH (100.0 g, 2500.2 mmol, 2.5 eq, 60%) in portions at 0° C. under nitrogen atmosphere. The reaction mixture was stirred overnight at 45° C. under nitrogen atmosphere. The resulting mixture was quenched by the addition of HCl (1 M, 300 mL) and extracted with EtOAc (2×500 mL). The combined organic layer was washed with brine (1000 mL), dried over anhydrous Na₂SO₄. After filtration, the filtrate was concentrated under vacuum. The crude residue was applied onto a silica gel column and eluted with ethyl acetate/petroleum ether=1:2 to give methyl 4-oxooxane-3-carboxylate as a yellow oil (20.0 g, 12.7%).

Synthesis of methyl 4-hydroxyoxane-3-carboxylate: Into a 1 L 3-necked round-bottom flask, were placed methyl 4-oxooxane-3-carboxylate (20.0 g, 126.5 mmol, 1.0 eq), methanol (300 mL). After that, to the above mixture was added NaBH₄ (5.8 g, 153.3 mmol, 1.2 eq) in portions at −78° C. The reaction mixture was stirred for additional 0.5 h at −78° C. The resulting mixture was quenched by the addition of aqueous NH₄Cl and extracted with CH₂Cl₂ (2×200 mL). The combined organic layer was washed with brine (500 mL), dried over anhydrous Na₂SO₄. After filtration, the filtrate was concentrated under vacuum to give methyl 4-hydroxyoxane-3-carboxylate as a colorless oil (9.7 g, 47.9%). ¹HNMR (300 MHz, Chloroform-d) δ 4.31-4.10 (m, 1H), 4.07-3.77 (m, 3H), 3.73 (s, 3H), 3.63 (dt, J=11.5, 4.5 Hz, 1H), 3.04 (s, 1H), 2.89-2.46 (m, 1H), 2.11-1.53 (m, 2H).

Synthesis of methyl 4-[(tert-butyldiphenylsilyl)oxy]oxane-3-carboxylate: Into a 250 mL round-bottom flask, were placed methyl 4-hydroxyoxane-3-carboxylate (6.5 g, 40.6 mmol, 1.0 eq), tetrahydrofuran (100 mL), pyridine (16.0 g, 202.3 mmol, 5.0 eq), AgNO₃ (8.3 g, 48.8 mmol, 1.2 eq), tert-butyl(chloro)diphenylsilane (14.5 g, 52.7 mmol, 1.3 eq). The reaction mixture was stirred for 3 hours at 25° C. under nitrogen atmosphere. The resulting mixture was quenched by the addition of water (300 mL) and extracted with EtOAc (2×100 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=1:10 to give methyl 4-[(tert-butyldiphenylsilyl)oxy]oxane-3-carboxylate as a colorless oil (9.2 g, 56.9%). ¹HNMR (400 MHz, Chloroform-d) δ 7.80-7.70 (m, 4H), 7.51-7.35 (m, 6H), 4.57 (q, J=3.2 Hz, 1H), 4.12 (dd, J=11.6, 10.5 Hz, 1H), 3.95 (dd, J=11.6, 4.4 Hz, 1H), 3.87 (td, J=11.0, 3.5 Hz, 1H), 3.66-3.55 (m, 1H), 3.51 (s, 3H), 2.65 (ddd, J=10.5, 4.4, 2.8 Hz, 1H), 1.66-1.50 (m, 2H), 1.08 (s, 9H).

Synthesis of 4-[(tert-butyldiphenylsilyl)oxy]oxane-3-carboxylic acid: Into a 250 mL round-bottom flask, were placed methyl 4-[(tert-butyldiphenylsilyl)oxy]oxane-3-carboxylate (9.0 g, 22.6 mmol, 1.0 eq), methanol (30 mL), tetrahydrofuran (20 mL), water (10 mL), LiOHH₂O (1.9 g, 45.3 mmol, 2.0 eq). The reaction mixture was stirred for 5 hours at 40° C. The resulting mixture was concentrated under vacuum. The residue was acidified to pH=5 with HCl (1 M) and extracted with EtOAc (2×100 mL). The combined organic layers were washed with brine (200 mL), dried over anhydrous Na₂SO₄. After filtration, the filtrate was concentrated under vacuum to give 4-[(tert-butyldiphenylsilyl)oxy]oxane-3-carboxylic acid as a grey solid (8.6 g, 99.0%). LC-MS (ESI, m/z) M+Na⁺: 407.

Synthesis of 4-[(tert-butyldiphenylsilyl)oxy]oxane-3-carboxamide (trans): Into a 250 mL round-bottom flask, were placed 4-[(tert-butyldiphenylsilyl)oxy]oxane-3-carboxylic acid (8.6 g, 22.4 mmol, 1.0 eq), tetrahydrofuran (100 mL), triethylamine (11.3 g, 111.7 mmol, 5.0 eq), NH₄Cl (3.1 g, 58.0 mmol, 2.6 eq), HATU (10.2 g, 26.8 mmol, 1.2 eq). The reaction mixture was stirred overnight at 25° C. The resulting mixture was quenched by the addition of water (300 mL) and extracted with ethyl acetate (2×100 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give 4-[(tert-butyldiphenylsilyl)oxy]oxane-3-carboxamide (trans) as a white solid (4.5 g, 52.5%). ¹HNMR (300 MHz, Chloroform-d) δ 7.77-7.65 (m, 4H), 7.54-7.35 (m, 6H), 6.20 (s, 1H), 5.74 (s, 1H), 4.24 (td, J=8.0, 4.3 Hz, 1H), 4.08 (dd, J=11.9, 3.9 Hz, 1H), 3.83 (dt, J=11.7, 4.7 Hz, 1H), 3.59 (dd, J=11.9, 8.1 Hz, 1H), 3.32 (ddd, J=11.9, 8.9, 3.4 Hz, 1H), 2.51 (td, J=7.9, 3.9 Hz, 1H), 1.70-1.43 (m, 2H), 1.08 (s, 9H).

Synthesis of 3-(aminomethyl)oxan-4-ol (trans): Into a 100 mL round-bottom flask, were placed 4-[(tert-butyldiphenylsilyl)oxy]oxane-3-carboxamide (trans) (3.0 g, 7.8 mmol, 1.0 eq), tetrahydrofuran (30 mL). After that, to the above mixture was added LAlH₄ (893 mg, 23.5 mmol, 3.0 eq) in portions at 0° C. The reaction mixture was stirred overnight at 25° C. The resulting mixture was quenched by the addition of Na₂SO₄·10H₂O at 0° C. The solids were filtered out and the filtrate was concentrated under vacuum to give 3-(aminomethyl)oxan-4-ol (trans) as a white solid (1.0 g, crude). LC-MS (ESI, m/z) M+1: 132.

Synthesis of N-[(4-hydroxyoxan-3-yl)methyl]-4-methylbenzenesulfonamide (trans): Into a 100 mL 3-necked round-bottom flask, were placed 3-(aminomethyl)oxan-4-ol (trans) (1.0 g, 7.6 mmol, 1.0 eq), dichloromethane (20 mL), triethylamine (1.5 g, 14.8 mmol, 1.9 eq). After that, to the above mixture was added 4-methylbenzenesulfonyl chloride (1.6 g, 8.4 mmol, 1.1 eq) in portions at 0° C. The reaction mixture was stirred for additional 2 hours at 25° C. The resulting mixture was quenched by the addition of water (200 mL) and extracted with CH₂Cl₂ (2×50 mL). The combined organic layer was washed with brine (100 mL), dried over anhydrous Na₂SO₄. After filtration, the filtrate was concentrated under vacuum. The crude residue was applied onto a silica gel column and eluted with ethyl acetate/petroleum ether=2:1 to give N-[(4-hydroxyoxan-3-yl)methyl]-4-methylbenzenesulfonamide (trans) as a colorless oil (2.0 g, 91.9%). ¹HNMR (400 MHz, Chloroform-d) δ 7.79-7.73 (m, 2H), 7.34 (d, J=7.9 Hz, 2H), 4.01-3.92 (m, 1H), 3.90-3.82 (m, 1H), 3.66 (td, J=10.0, 4.6 Hz, 1H), 3.37 (td, J=11.8, 2.3 Hz, 1H), 3.14-3.02 (m, 2H), 2.89 (dd, J=13.1, 6.4 Hz, 1H), 2.46 (s, 3H), 1.90 (ddt, J=12.8, 4.6, 2.3 Hz, 1H), 1.74 (dt, J=10.0, 5.6 Hz, 1H), 1.72-1.56 (m, 1H).

Synthesis of N-({4-[(5-bromo-1-{[2-(trimethylsilyl)ethoxy]methyl}pyrrolo[2,3-b]pyridin-6-yl)oxy]oxan-3-yl}methyl)-4-methylbenzenesulfonamide (trans): Into a 250 mL round-bottom flask, were placed N-[(4-hydroxyoxan-3-yl)methyl]-4-methylbenzenesulfonamide (trans) (2.0 g, 7.0 mmol, 1.0 eq), tetrahydrofuran (30 mL). After that, to the above mixture was added NaH (842 mg, 21.1 mmol, 3.0 eq., 60%) in portions at 0° C. The reaction mixture was stirred for additional 30 min at 0° C., followed by the addition of 5-bromo-6-fluoro-1-{[2-(trimethylsilyl)ethoxy]methyl}pyrrolo[2,3-b]pyridine (2.4 g, 7.0 mmol, 1.0 eq) dropwise at 0° C. The reaction mixture was stirred overnight at 60° C., and then quenched by the addition of aqueous NH₄Cl and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give N-({4-[(5-bromo-1-{[2-(trimethylsilyl)ethoxy]methyl}pyrrolo[2,3-b]pyridin-6-yl)oxy]oxan-3-yl}methyl)-4-methylbenzenesulfonamide (trans) as a light yellow oil (1.6 g, 37.4%). ¹HNMR (300 MHz, Chloroform-d) δ 8.06 (s, 1H), 7.64 (d, J=8.3 Hz, 2H), 7.18 (dd, J=5.9, 2.2 Hz, 3H), 6.42 (d, J=3.6 Hz, 1H), 5.57 (d, J=1.6 Hz, 2H), 5.14 (td, J=8.6, 4.2 Hz, 1H), 4.01 (dd, J=11.7, 4.3 Hz, 2H), 3.65-3.46 (m, 2H), 3.40 (dd, J=11.8, 8.8 Hz, 1H), 3.03 (dt, J=14.8, 8.5 Hz, 2H), 2.37 (s, 3H), 2.33-2.24 (m, 1H), 2.20-2.09 (m, 1H), 1.83-1.75 (m, 2H), 0.96-0.82 (m, 2H), −0.05 (s, 9H).

Synthesis of 10-(4-methylbenzenesulfonyl)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene (trans): Into a 100 mL round-bottom flask, were placed N-({4-[(5-bromo-1-{[2-(trimethylsilyl)ethoxy]methyl}pyrrolo[2,3-b]pyridin-6-yl)oxy]oxan-3-yl}methyl)-4-methylbenzenesulfonamide (trans) (900 mg, 1.5 mmol, 1.0 eq), Dimethyl sulfoxide (30 mL), K₂CO₃ (612 mg, 4.4 mmol, 3.0 eq), CuI (140 mg, 0.7 mmol, 0.5 eq), picolinic acid (91 mg, 0.7 mmol, 0.5 eq). The reaction mixture was stirred overnight at 135° C. The resulting mixture was quenched by the addition of water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=3:2 to give 10-(4-methylbenzenesulfonyl)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene (trans) as a yellow oil (548 mg, 70.2%). LC-MS (ESI, m/z) M+1: 530.

Synthesis of (3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene and (3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene: Into a 40 mL vial purged and maintained with an inert atmosphere of nitrogen, were placed Na (143 mg, 6.2 mmol, 6.0 eq), Naphthalene (796 mg, 6.2 mmol, 6.0 eq), ethylene glycol dimethyl ether (10 mL). The mixture was stirred for 40 min at 25° C. until the formation of Na/naphthalene was complete. At the same time, another 100 mL 3-necked round-bottom flask purged and maintained with an inert atmosphere of nitrogen, were placed 10-(4-methylbenzenesulfonyl)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene (trans) (548 mg, 1.0 mmol, 1.0 eq), tetrahydrofuran (10 mL). This was followed by the addition of the above solution at −78° C. The resulting solution was stirred for 3 hours at 25° C. The reaction was then quenched by the addition of aqueous NH₄Cl (300 mL) and extracted with EtOAc (3×100 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=3:2. The crude product was purified by Prep-SFC using the following conditions. Finally, (3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene was obtained as a brown oil (100 mg, 25.7%) and (3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene was obtained as a brown oil (80 mg, 20.6%). 11A, T_{R}=4.4 min in CHIRAL-SFC, Column: CHIRAL ART Cellulose-SC, 3*25 cm, 5 um; Mobile Phase A: CO2, Mobile Phase B: MeOH; Flow rate: 80 mL/min; Gradient: 30% B, Wave Length: 220 nm. 11B, T_{R}=6.8 min in CHIRAL-SFC, Column: CHIRAL ART Cellulose-SC, 3*25 cm, 5 um; Mobile Phase A: CO2, Mobile Phase B: MeOH; Flow rate: 80 mL/min; Gradient: 30% B, Wave Length: 220 nm. LC-MS (ESI, m/z) M+1: 376. ¹HNMR (300 MHz, Chloroform-d) δ 7.40 (s, 1H), 7.22 (d, J=3.5 Hz, 1H), 6.35 (d, J=3.5 Hz, 1H), 5.66 (d, J=10.7 Hz, 1H), 5.49 (d, J=10.7 Hz, 1H), 4.11 (dd, J=11.7, 4.8 Hz, 1H), 3.92 (dd, J=11.5, 4.4 Hz, 1H), 3.80-3.73 (m, 1H), 3.63-3.48 (m, 2H), 3.41 (td, J=12.3, 2.2 Hz, 1H), 3.27 (dd, J=12.5, 4.0 Hz, 1H), 3.07 (t, J=11.4 Hz, 1H), 2.58-2.49 (m, 1H), 2.36-2.16 (m, 2H), 2.06 (tdd, J=12.8, 11.2, 5.0 Hz, 1H), 0.90 (dp, J=9.8, 7.1 Hz, 2H), −0.06 (s, 9H).

### Example INT_28 Preparation of (6aR,9aS)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine and (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine

Synthesis of (3S,4R)-4-hydroxyoxolane-3-carbonitrile (racemate): Into a 500 mL 3-necked round-bottom flask were added 3,6-dioxabicyclo[3-1-0]hexane (10.0 g, 116.2 mmol, 1.0 eq) and toluene (100 mL) at 25° C. After that, to the above mixture was added cyanodiethylaluminum (197.47 mL, 197.469 mmol, 1.7 eq) dropwise over 30 min at 25° C. The reaction mixture was stirred for 16 hours at 50° C. The resulting mixture was concentrated under vacuum to give (3S,4R)-4-hydroxyoxolane-3-carbonitrile (racemate) as a coloress oil (6.0 g, crude). GC-MS (ESI, m/z) M+1: 114.

Synthesis of (3R,4S)-4-(aminomethyl)oxolan-3-ol (racemate): Into a 250 mL round-bottom flask were added (3S,4R)-4-hydroxyoxolane-3-carbonitrile (6.0 g, crude, 53.0 mmol, 1.0 eq), MeOH (100 mL) and Raney Ni (0.2 g, 2.7 mmol, 0.05 eq) at 25° C. The resulting mixture was stirred for 3 hours at 25° C. under hydrogen atmosphere. The resulting mixture was filtered, the filter cake was washed with MeOH (2×50 mL).

After filtration, the filtrate was concentrated under vacuum to give (3R,4S)-4-(aminomethyl)oxolan-3-ol (racemate) as a colorless oil (4.0 g, crude).

Synthesis of N-{[(3S,4R)-4-hydroxyoxolan-3-yl]methyl}-4-methylbenzenesulfonamide (racemate): Into a 250 mL 3-necked round-bottom flask were added (3R,4S)-4-(aminomethyl)oxolan-3-ol (4.5 g, 38.4 mmol, 1.0 eq), DCM (100 mL) and TEA (9.7 g, 96.0 mmol, 2.5 eq) at 25° C. After that, to the above mixture was added p-toluenesulfonyl chloride (7.3 g, 38.4 mmol, 1.0 eq) in portions over 10 min at 0° C. The reaction mixture was stirred for 4 hours at 25° C. The resulting mixture was quenched by the addition of water (10 mL) at 0° C. The resulting mixture was extracted with CH₂Cl₂ (2×20 mL). The combined organic layers were washed with brine (20 mL), dried over anhydrous Na₂SO₄. After filtration, the filtrate was concentrated under vacuum. The crude residue was applied onto a silica gel column and eluted with ethyl acetate/petroleum ether=1:1 to give N-{[(3S,4R)-4-hydroxyoxolan-3-yl]methyl}-4-methylbenzenesulfonamide (racemate) as a white solid (4.0 g, 38.4%). LC-MS (ESI, m/z) M+1: 272.

Synthesis of N-(((3S,4R)-4-((5-bromo-1-((2-(trimethylsilyl)ethoxy)methyl)-1H-pyrrolo[2,3-b]pyridin-6-yl)oxy)tetrahydrofuran-3-yl)methyl)-4-methylbenzenesulfonamide (trans): Into a 250 mL round-bottom flask, were placed N-(((3S,4R)-4-hydroxytetrahydrofuran-3-yl)methyl)-4-methylbenzenesulfonamide (2.0 g, 7.0 mmol, 1.0 eq), tetrahydrofuran (30 mL). After that, to the above mixture was added NaH (842 mg, 21.1 mmol, 3.0 eq., 60%) in portions at 0° C. The reaction mixture was stirred for additional 30 min at 0° C. To the above mixture was added 5-bromo-6-fluoro-1-{[2-(trimethylsilyl)ethoxy]methyl}pyrrolo[2,3-b]pyridine (2.4 g, 7.0 mmol, 1.0 eq) dropwise at 0° C. The reaction mixture was stirred overnight at 60° C. The resulting mixture was quenched by the addition of aqueous NH₄Cl and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give N-(((3S,4R)-4-((5-bromo-1-((2-(trimethylsilyl)ethoxy)methyl)-1H-pyrrolo[2,3-b]pyridin-6-yl)oxy)tetrahydrofuran-3-yl)methyl)-4-methylbenzenesulfonamide (trans) as a light yellow oil (1.6 g, 37.4%). LC-MS (ESI, m/z) M+1: 596/598.

Synthesis of (6aS,9aR)-5-tosyl-1-((2-(trimethylsilyl)ethoxy)methyl)-5,6,6a,7,9,9a-hexahydro-1H. furo[3,4-f]pyrrolo[3′,2′:5,6]pyrido[2,3-b][1,4]oxazepine (trans): Into a 100 mL round-bottom flask, were placed N-(((3S,4R)-4-((5-bromo-1-((2-(trimethylsilyl)ethoxy)methyl)-1H-pyrrolo[2,3-b]pyridin-6-yl)oxy)tetrahydrofuran-3-yl)methyl)-4-methylbenzenesulfonamide (trans) (900 mg, 1.5 mmol, 1.0 eq), Dimethyl sulfoxide (30 mL), K₂CO₃ (612 mg, 4.4 mmol, 3.0 eq), CuI (140 mg, 0.7 mmol, 0.5 eq), picolinic acid (91 mg, 0.7 mmol, 0.5 eq). The reaction mixture was stirred overnight at 135° C. The resulting mixture was quenched by the addition of water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=3:2 to give (6aS,9aR)-5-tosyl-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans) as a yellow oil (548 mg, 70.2%). LC-MS (ESI, m/z) M+1: 516.

Synthesis of (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans): Into a 40 mL vial purged and maintained with an inert atmosphere of nitrogen, were placed Na (143 mg, 6.2 mmol, 6.0 eq), Naphthalene (796 mg, 6.2 mmol, 6.0 eq), ethylene glycol dimethyl ether (10 mL). The reaction mixture was stirred for 40 min at 25° C. until the formation of Na/naphthalene was complete. At the same time, another 100 mL 3-necked round-bottom flask purged and maintained with an inert atmosphere of nitrogen, were placed (6aS,9aR)-5-tosyl-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans) (548 mg, 1.0 mmol, 1.0 eq) and tetrahydrofuran (10 mL). This was followed by the addition of the above solution at −78° C. The resulting solution was stirred for 3 hours at 25° C. The reaction was then quenched by the addition of aqueous NH₄Cl (300 mL) and extracted with EtOAc (3×100 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=3:2. The crude product was purified by Prep-SFC to afford (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans) as a brown oil (200 mg, 50.7%). LC-MS (ESI, m/z) M+1: 362.

Synthesis of (6aR,9aS)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine and (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepane: 200 mg of (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans) was purified by Chiral-Prep-HPLC using the following conditions: Column: CHIRALPAK IH, 3*25 cm, 5 μm; mobile phase A: CO₂; mobile phase B: MeOH—Preparative; Flow rate: 80 mL/min; Gradient: isocratic 30% B; Wave Length: 220 nm. Finally, 90 mg of (6aR,9aS)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine was obtained as a yellow oil and 96 mg of (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine was obtained as a yellow oil. LC-MS (ESI, m/z) M+1: 362. 6A, T_{R}=1.342 min in CHIRAL-HPLC, Column: CHIRALPAK IH, 3*25 cm, 5 μm. mobile phase A: CO₂; mobile phase B: MeOH—Preparative, Pump Mode: Low pressure gradient, Conc. of Pump B:30.0%, Oven Temperature: 35° C. LC-MS (ESI, m/z) M+1: 362. 6B, T_{R}=1.559 min in CHIRAL-HPLC, Column: CHIRALPAK IH, 3*25 cm, 5 μm. mobile phase A: CO₂; mobile phase B: MeOH—Preparative, Pump Mode: Low pressure gradient, Conc. of Pump B:30.0%, Oven Temperature: 35° C.

### Example INT_29 Preparation of (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene

Synthesis of N-((3R,4S)-4-hydroxytetrahydro-2H-pyran-3-yl)-4-methylbenzenesulfonamide: Into a 100 mL 3-necked round-bottom flask, were placed (3R,4S)-3-aminotetrahydro-2H-pyran-4-ol (1.0 g, 8.5 mmol, 1.0 eq), dichloromethane (20 mL), triethylamine (2.1 g, 21.3 mmol, 2.5 eq). After that, to the above mixture was added 4-methylbenzenesulfonyl chloride (1.6 g, 9.4 mmol, 1.1 eq) in portions at 0° C. The reaction mixture was stirred for 4 hours at 25° C. under nitrogen atmosphere. The resulting mixture was quenched by the addition of water (100 mL) and extracted with CH₂Cl₂ (2×20 mL). The combined organic layer was washed with brine (20 mL), dried over anhydrous Na₂SO₄. After filtration, the filtrate was concentrated under vacuum. The crude residue was applied onto a silica gel column and eluted with ethyl acetate/petroleum ether=1:1 to give N-((3R,4S)-4-hydroxytetrahydro-2H-pyran-3-yl)-4-methylbenzenesulfonamide as a white solid (1.5 g, 64.7%). LC. MS (ESI, m/z) M+1: 272.

Synthesis of N-[(3R,4S)-3-[(5-bromo-1-{[2-(trimethylsilyl)ethoxy]methyl}pyrrolo[2,3-b]pyridin-6-yl)oxy]oxan-4-yl]-4-methylbenzenesulfonamide: Into a 100 mL round-bottom flask, were placed N-[(3R,4S)-3-hydroxyoxan-4-yl]-4-methylbenzenesulfonamide (800 mg, 2.9 mmol, 1.0 eq), tetrahydrofuran (10 mL). After that, to the above mixture was added NaH (354 mg, 8.8 mmol, 3.0 eq, 60%) in portions at 0° C. The reaction mixture was stirred for additional 30 min at 0° C. To the above mixture was added 5-bromo-6-fluoro-1-{[2-(trimethylsilyl)ethoxy]methyl}pyrrolo[2,3-b]pyridine (1.0 g, 2.9 mmol, 1.0 eq) dropwise at 0° C. The reaction mixture was stirred overnight at 50° C. The resulting mixture was quenched by the addition of aqueous NH₄Cl and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:2 to give N-[(3R,4S)-3-[(5-bromo-1-{[2-(trimethylsilyl)ethoxy]methyl}pyrrolo[2,3-b]pyridin-6-yl)oxy]oxan-4-yl]-4-methylbenzenesulfonamide as a white solid (400 mg, 22.7%). LC-MS (ESI, m/z) M+1: 596/598. ¹HNMR (300 MHz, Chloroform-d) δ 7.99 (s, 1H), 7.39-7.32 (m, 2H), 7.22 (d, J=3.6 Hz, 1H), 7.04 (d, J=8.0 Hz, 2H), 6.44 (d, J=3.6 Hz, 1H), 5.67-5.50 (m, 2H), 4.91 (td, J=9.8, 5.1 Hz, 1H), 4.32-4.07 (m, 1H), 3.95 (dd, J=12.0, 4.5 Hz, 1H), 3.73-3.51 (m, 2H), 3.40 (ddd, J=21.3, 11.7, 10.0 Hz, 3H), 2.37 (s, 3H), 2.05-1.85 (m, 1H), 1.33-1.22 (m, 1H), 1.08-0.93 (m, 2H), −0.00 (s, 9H).

Synthesis of (3R,8S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 250 mL round-bottom flask, were placed N-[(3R,4S)-3-[(5-bromo-1-{[2-(trimethylsilyl)ethoxy]methyl}pyrrolo[2,3-b]pyridin-6-yl)oxy]oxan-4-yl]-4-methylbenzenesulfonamide (400 mg, 0.7 mmol, 1.0 eq), N,N-dimethylformamide (5 mL), K₂CO₃ (278 mg, 2.0 mmol, 3.0 eq), CuI (77 mg, 0.4 mmol, 0.6 eq), 1,10-phenanthroline (72 mg, 0.4 mmol, 0.6 eq). The reaction mixture was stirred overnight at 120° C. under nitrogen atmosphere. The resulting mixture was quenched by the addition of water (400 mL) and extracted with EtOAc (2×200 mL). The combined organic layer was washed with brine (500 mL), dried over anhydrous Na₂SO₄. After filtration, the filtrate was concentrated under vacuum. The crude residue was applied onto a silica gel column and eluted with ethyl acetate/petroleum ether=2:3 to give (3R,8S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene as a colorless oil (300 mg, 86.7%). LC-MS (ESI, m/z) M+1: 516. ¹HNMR (400 MHz, Chloroform-d) δ 8.44 (s, 1H), 7.29-7.23 (m, 3H), 7.13 (d, J=8.2 Hz, 2H), 6.57 (d, J=3.6 Hz, 1H), 5.55 (s, 2H), 4.22 (dd, J=10.9, 4.6 Hz, 1H), 4.06 (dd, J=11.9, 4.4 Hz, 1H), 3.63 (dd, J=10.0, 4.5 Hz, 1H), 3.58-3.46 (m, 4H), 3.31 (t, J=10.3 Hz, 1H), 2.72-2.66 (m, 1H), 2.36 (s, 3H), 2.12 (td, J=12.6, 4.6 Hz, 1H), 1.31-1.23 (m, 2H), −0.04 (s, 9H).

Synthesis of (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 40 mL vial purged and maintained with an inert atmosphere of nitrogen, were placed Na (93 mg, 4.0 mmol, 7.0 eq), naphthalene (521 mg, 4.0 mmol, 7.0 eq), ethylene glycol dimethyl ether (3 mL). The mixture was stirred for 40 minutes at 25° C. until the formation of Na/naphthalene was complete. At the same time, another 250 mL 3-necked round-bottom flask purged and maintained with an inert atmosphere of nitrogen, were placed (3R,8S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (300 mg, 0.6 mmol, 1.0 eq), tetrahydrofuran (10 mL). This was followed by the addition of the above solution at −78° C. The resulting solution was stirred for 3 hours at 25° C. The reaction was then quenched by the addition of aqueous NH₄Cl (300 mL) and extracted with EtOAc (3×200 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=1:1 to give (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene as a yellow oil (120 mg, 57.06%). LC-MS (ESI, m/z) M+1: 362. ¹NHMR (300 MHz, Chloroform-d) δ 7.50 (s, 1H), 7.20 (d, J=3.5 Hz, 1H), 6.36 (d, J=3.6 Hz, 1H), 5.57 (s, 2H), 4.37 (dd, J=10.9, 5.0 Hz, 1H), 4.24-3.98 (m, 2H), 3.66-3.26 (m, 5H), 2.39-2.11 (m, 1H), 2.10-1.84 (m, 1H), 0.96-0.86 (m, 2H), −0.04 (s, 9H).

### Example INT_30 Preparation of (3R)-3-(2-isopropylphenyl)morpholine and (3S)-3-(2-isopropylphenyl)morpholine

Synthesis of 2-amino-2-(2-bromophenyl)acetonitrile hydrochloride: Into a 1 L round-bottom flask, were placed 2-bromobenzaldehyde (200.0 g, 1080.9 mmol, 1.0 eq), TMSCN (134.1 g, 1351.2 mmol, 1.3 eq), ZnI₂ (138.0 g, 432.4 mmol, 0.4 eq). The reaction mixture was stirred for 6 hours at 25° C. After that, to the above mixture was added NH₃(gas) in methanol (900 mL, 7 M). The reaction mixture was transferred into a 5 L pressure tank reactor and stirred for additional 16 hours at 50° C. The resulting mixture was concentrated under vacuum. To the above mixture was added HCl in dioxane (200 mL, 4 M) in portions at 25° C. The precipitated solids were collected by filtration. Finally, 2-amino-2-(2-bromophenyl)acetonitrile hydrochloride was obtained as a white solid (160.0 g, 70.1%). LC-MS (ESI, m/z) M+1: 210/212.

Synthesis of amino(2-bromophenyl)acetic acid hydrochloride: A mixture of 2-amino-2-(2-bromophenyl)acetonitrile hydrochloride (82.0 g, 388.5 mmol, 1.0 eq) in HCl (820 mL, 6 M) was stirred for 16 hours at 100° C. The resulting mixture was adjusted to pH=6 with aqueous NaOH. The precipitated solids were collected by filtration. Finally, amino(2-bromophenyl)acetic acid hydrochloride was obtained as a yellow solid (55.0 g, 61.5%). LC-MS (ESI, m/z) M+1: 229/231.

Synthesis of 2-amino-2-(2-bromophenyl)ethan-1-ol: Into a 500 mL 3-necked round-bottom flask, were placed amino(2-bromophenyl)acetic acid hydrochloride (8.0 g, 34.8 mmol, 1.0 eq), tetrahydrofuran (80 mL). After that, to the above mixture was added BH₃-THF (140 mL, 140.0 mmol, 4.0 eq) dropwise at 0° C. The reaction mixture was stirred overnight at 25° C. The resulting mixture was then quenched by the addition of HCl (1 M). The resulting mixture was concentrated under vacuum. The residue was basified to pH=12 with NaOH. The resulting mixture was extracted with CH₂Cl₂ (3×50 mL). The combined organic layers were dried over anhydrous Na₂SO₄. After filtration, the filtrate was concentrated under vacuum. The crude product was re-crystallized from ethyl acetate/petroleum ether=1:8 to give 2-amino-2-(2-bromophenyl)ethan-1-ol as a brown solid (4.2 g, 55.9%). ¹HNMR (400 MHz, DMSO-d₆) δ 7.64 (dd, J=7.8, 1.8 Hz, 1H), 7.55 (dd, J=8.0, 1.3 Hz, 1H), 7.36 (td, J=7.5, 1.3 Hz, 1H), 7.17 (td, J=7.6, 1.8 Hz, 1H), 4.88 (s, 1H), 4.25 (dd, J=7.9, 4.0 Hz, 1H), 3.53 (dd, J=10.4, 4.0 Hz, 1H), 3.19 (dd, J=10.5, 7.9 Hz, 1H), 2.13 (s, 2H).

Synthesis of N-[1-(2-bromophenyl)-2-hydroxyethyl]-2-chloroacetamide: Into a 250 mL round-bottom flask, were placed 2-amino-2-(2-bromophenyl)ethanol (4.2 g, 19.4 mmol, 1.0 eq), dichloromethane (50 mL), triethylamine (3.0 g, 29.6 mmol, 1.5 eq). To the above mixture was added chloroacetyl chloride (2.3 g, 20.4 mmol, 1.05 eq) dropwise at 0° C. The reaction mixture was stirred for additional 2 hours at 25° C. The resulting mixture was quenched by the addition of water (200 mL) and extracted with CH₂Cl₂ (2×50 mL). The combined organic layer was washed with brine (200 mL), dried over anhydrous Na₂SO₄. After filtration, the filtrate was concentrated under vacuum to give N-[1-(2-bromophenyl)-2-hydroxyethyl]-2-chloroacetamide as a brown solid (5.1 g, 89.7%). ¹HNMR (300 MHz, DMSO-d₆) δ 8.77 (d, J=7.7 Hz, 1H), 7.59 (dd, J=7.9, 1.1 Hz, 1H), 7.49-7.32 (m, 2H), 7.21 (ddd, J=8.0, 6.6, 2.5 Hz, 1H), 5.18 (td, J=7.5, 4.3 Hz, 1H), 5.10 (t, J=5.6 Hz, 1H), 4.15 (s, 2H), 3.62 (dt, J=11.1, 4.8 Hz, 1H), 3.49 (ddd, J=11.1, 7.4, 5.9 Hz, 1H).

Synthesis of 5-(2-bromophenyl)morpholin-3-one: Into a 500 mL round-bottom flask, were placed N-[1-(2-bromophenyl)-2-hydroxyethyl]-2-chloroacetamide (5.1 g, 17.4 mmol, 1.0 eq), tetrahydrofuran (200 mL). After that, to the above mixture was added NaH (2.1 g, 52.5 mmol, 3.0 eq., 60%) in portions at 0° C. The reaction mixture was stirred overnight at 25° C. The resulting mixture was quenched by the addition of aqueous NH₄Cl (200 mL) and extracted with CH₂Cl₂ (2×100 mL). The combined organic layer was washed with brine (200 mL), dried over anhydrous Na₂SO₄. After filtration, the filtrate was concentrated under vacuum. The crude residue was applied onto a silica gel column and eluted with dichloromethane/methanol=10:1 to give 5-(2-bromophenyl)morpholin-3-one as a brown solid (3.6 g, 80.6%). ¹HNMR (300 MHz, DMSO-d₆) δ 8.47-8.40 (m, 1H), 7.65 (dd, J=7.9, 1.2 Hz, 1H), 7.52-7.37 (m, 2H), 7.29 (ddd, J=7.9, 6.9, 2.2 Hz, 1H), 4.90 (q, J=4.0 Hz, 1H), 4.12 (d, J=4.2 Hz, 2H), 4.09-3.96 (m, 1H), 3.60 (dd, J=11.7, 4.9 Hz, 1H).

Synthesis of 3-(2-bromophenyl)morpholine: Into a 250 mL round-bottom flask, were placed 5-(2-bromophenyl)morpholin-3-one (3.6 g, 14.1 mmol, 1.0 eq), tetrahydrofuran (40 mL). After that, to the above mixture was added LAlH₄ (1.6 g, 42.1 mmol, 3.0 eq) in portions at 0° C. The reaction mixture was stirred overnight at 25° C. The resulting mixture was then quenched with Na₂SO₄·10H₂O at 0° C. The solids were filtered out. The filtrate was concentrated under vacuum. The crude residue was applied onto a silica gel column and eluted with dichloromethane/methanol=19:1 to give 3-(2-bromophenyl)morpholine as a brown oil (1.7 g, 49.9%). LC-MS (ESI, m/z) M+1: 242/244.

Synthesis of tert-butyl 3-(2-bromophenyl)morpholine-4-carboxylate: Into a 100 mL round-bottom flask, were placed 3-(2-bromophenyl)morpholine (1.4 g, 5.8 mmol, 1.0 eq), dichloromethane (20 mL), triethylamine (1.2 g, 11.8 mmol, 2.0 eq), N,N-dimethylpyridin-4-amine (71 mg, 0.6 mmol, 0.1 eq), di-tert-butyl dicarbonate (1.5 g, 6.9 mmol, 1.2 eq). The reaction mixture was stirred overnight at 25° C. The resulting mixture was concentrated under vacuum. The crude residue was applied onto a silica gel column and eluted with ethyl acetate/petroleum ether=1:10 to give tert-butyl 3-(2-bromophenyl)morpholine-4-carboxylate as a light yellow oil (1.2 g, 63.1%). LC-MS (ESI, m/z) M-t-Bu+1: 242/244.

Synthesis of tert-butyl 3-[2-(prop-1-en-2-yl)phenyl]morpholine-4-carboxylate: Into a 100 mL round-bottom flask, were placed tert-butyl 3-(2-bromophenyl)morpholine-4-carboxylate (1.2 g, 3.6 mmol, 1.0 eq) and 4,4,5,5-tetramethyl-2-(prop-1-en-2-yl)-1,3,2-dioxaborolane (1.5 g, 8.9 mmol, 2.4 eq), dioxane (27 mL), water (3 mL), K₂CO₃ (1.5 g, 10.9 mmol, 3.0 eq), Pd(dppf)Cl₂·CH₂Cl₂ (300 mg, 0.4 mmol, 0.1 eq). The reaction mixture was stirred overnight at 80° C. under nitrogen atmosphere. The resulting mixture was quenched by the addition of water (200 mL) and extracted with ethyl acetate (3×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:10 to give tert-butyl 3-[2-(prop-1-en-2-yl)phenyl]morpholine-4-carboxylate as a light yellow oil (800 mg, 72.2%). LC-MS (ESI, m/z) M-Boc+1: 204.

Synthesis of tert-butyl 3-(2-isopropylphenyl)morpholine-4-carboxylate: Into a 50 mL pressure tank reactor, were placed tert-butyl 3-[2-(prop-1-en-2-yl)phenyl]morpholine-4-carboxylate (800 mg, 2.6 mmol, 1.0 eq), methanol (20 mL), Pd/C (10%, 100 mg). The mixture was hydrogenated at 50° C. under 20 atm of hydrogen pressure for overnight, filtered through a Celite pad and concentrated under vacuum. The crude residue was applied onto a silica gel column and eluted with ethyl acetate/petroleum ether=1:10 to give tert-butyl 3-(2-isopropylphenyl)morpholine-4-carboxylate as a colorless oil (600 mg, 74.5%). ¹HNMR (400 MHz, Chloroform-d) δ 7.69 (dd, J=7.7, 1.3 Hz, 1H), 7.35 (dd, J=7.8, 1.6 Hz, 1H), 7.29 (td, J=7.5, 1.4 Hz, 1H), 7.18 (td, J=7.5, 1.6 Hz, 1H), 5.34-5.25 (m, 1H), 4.14 (dd, J=11.9, 2.3 Hz, 1H), 3.98-3.88 (m, 2H), 3.81 (dt, J=13.7, 2.6 Hz, 1H), 3.68 (td, J=11.3, 3.2 Hz, 1H), 3.40-3.23 (m, 2H), 1.43 (s, 9H), 1.26 (d, J=6.9 Hz, 3H), 1.21 (d, J=6.8 Hz, 3H).

Synthesis of (3R)-3-(2-isopropylphenyl)morpholine and (3S)-3-(2-isopropylphenyl)morpholine: A solution of tert-butyl 3-(2-isopropylphenyl)morpholine-4-carboxylate (600 mg, 1.9 mmol, 1.0 eq) in HCl (gas) in 1,4-dioxane (10 mL) was stirred for 1 hour at 25° C. The reaction mixture was concentrated under vacuum. The resulting mixture was diluted with saturated aqueous NaHCO₃ and extracted with CH₂Cl₂ (2×50 mL). The combined organic layer was washed with brine (100 mL), dried over anhydrous Na₂SO₄. After filtration, the filtrate was concentrated under vacuum. The crude product was purified by Prep-CHIRAL-HPLC using the following conditions. Finally, (3R)-3-(2-isopropylphenyl)morpholine was obtained as a light yellow oil (99 mg, 24.5%) and (3S)-3-(2-isopropylphenyl)morpholine was obtained as a light yellow oil (93 mg, 23.0%). 9A, T_{R}=7 min in CHIRAL-HPLC, Column: CHIRAL ART Amylose-C NEO, 3*25 cm, 5 um; Mobile Phase A: Hexane (0.2% DEA), Mobile Phase B: EtOH; Flow rate: 35 mL/min; Gradient: 10% B to 10% B in 10 min, Wave Length: 220/254 nm. 9B, T_{R}=8 min in CHIRAL-HPLC, Column: CHIRAL ART Amylose-C NEO, 3*25 cm, 5 um; Mobile Phase A: Hexane (0.2% DEA), Mobile Phase B: EtOH; Flow rate: 35 mL/min; Gradient: 10% B to 10% B in 10 min, Wave Length: 220/254 nm.

### Example INT_31 Preparation of (R)-2-amino-2-(o-tolyl)ethan-1-ol

Synthesis of (R)-2-(((R)-1-(4-methoxyphenyl)ethyl)amino)-2-(o-tolyl)acetonitrile hydrochloride: Into a 1 L 3-necked round-bottom flask were added 2-methylbenzaldehyde (20.0 g, 166.5 mmol, 1.0 eq), (R)-1-(4-methoxyphenyl)ethan-1-amine hydrochloride (31.2 g, 166.5 mmol, 1.0 eq), MeOH (200 mL), water (200 mL) at 25° C. After that, to the above mixture was added NaCN (9.0 g, 183.1 mmol, 1.1 eq) in portions at 25° C. The resulting mixture was stirred for additional 14 hours at 25° C. The resulting mixture was filtered, the filter cake was washed with water (3×50 mL). The residue was dissolved in dichloromethane (300 mL). The organic layers were washed with water (3×50 mL) and brine (50 mL), dried over anhydrous Na₂SO₄. After filtration, the filtrate was concentrated under reduced pressure. (2R)-2-{[(1R)-1-(4-methoxyphenyl)ethyl]amino}-2-(2-methylphenyl)acetonitrile hydrochloride (30.0 g, crude) was dissolved in Et₂O (3 L). To the above mixture was added HCl in MeOH (1 M, 200 mL) dropwise at 25° C. The resulting mixture was stirred for additional 14 hours at 25° C. The precipitated solids were collected by filtration and washed with Et₂O (3×200 mL). Finally, (R)-2-(((R)-1-(4-methoxyphenyl)ethyl)amino)-2-(o-tolyl)acetonitrile hydrochloride (30.2 g, 56.9%) was obtained as a white solid. ¹HNMR (300 MHz, DMSO-d₆) δ 7.59-7.50 (m, 1H), 7.38-7.12 (m, 6H), 6.99-6.84 (m, 1H), 4.36 (d, J=11.2 Hz, 1H), 3.96 (tt, J=6.5, 3.3 Hz, 1H), 3.76 (s, 3H), 3.25 (dd, J=11.2, 2.7 Hz, 1H), 2.07 (s, 3H), 1.35-1.26 (m, 3H).

Synthesis of (R)-2-amino-2-(o-tolyl)acetic acid: Into a 1 L round-bottom flask were added (R)-2-(((R)-1-(4-methoxyphenyl)ethyl)amino)-2-(o-tolyl)acetonitrile hydrochloride (30 g, 94.7 mmol, 1 eq) and HCl (6 M, 300 mL) at 25° C. The resulting mixture was stirred for 4 hours at 90° C. The resulting mixture was extracted with Et₂O (3×50 mL). The aqueous phase was concentrated under reduced pressure to give (R)-2-amino-2-(o-tolyl)acetic acid (13.0 g, 83.1%) as a white solid. ¹HNMR (400 MHz, DMSO-d₆) δ 13.54 (bs, 1H), 8.88 (bs, 2H), 7.40-7.30 (m, 1H), 7.33-7.23 (m, 3H), 5.13 (s, 1H), 2.45 (s, 3H).

Synthesis of (R)-2-amino-2-(o-tolyl)ethan-1-ol: To a stirred solution of (R)-2-amino-2-(o-tolyl)acetic acid (6.0 g, 36.3 mmol, 1.0 eq) in THE (100 mL) were added BH₃-THF (148 mL, 148.0 mmol, 4.0 eq) dropwise at 25° C. under nitrogen atmosphere. The resulting mixture was stirred overnight at 25° C. The reaction was quenched with HCl (1 M). The resulting mixture was concentrated under vacuum. The residue was basified to pH=12 with NaOH (4 M). The resulting mixture was extracted with CH₂Cl₂ (3×100 mL). The combined organic layers were washed with brine (300 mL), dried over anhydrous Na₂SO₄. After filtration, the resulting mixture was concentrated under vacuum to give (R)-2-amino-2-(o-tolyl)ethan-1-ol as a white solid (4.5 g, 81.9%). ¹HNMR (300 MHz, DMSO-d₆) δ 7.51-7.45 (m, 1H), 7.19-7.11 (m, 1H), 7.14-7.05 (m, 2H), 4.77 (s, 1H), 4.11 (dd, J=8.4, 4.2 Hz, 1H), 3.44 (dd, J=10.5, 4.2 Hz, 1H), 3.21 (dd, J=10.5, 8.4 Hz, 1H), 2.31 (s, 3H).

### Example INT_32 Preparation of N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide

Synthesis of 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide: Into a 40 mL vial, were placed 2-bromo-4-{2-oxo-7-azaspiro[3.5]nonan-7-yl}benzoic acid (470 mg, 1.4 mmol, 1.0 eq), 3-nitro-4-({[(1r,4r)-4-hydroxy-4-methylcyclohexyl]methyl}amino)benzenesulfonamide (477 mg, 1.4 mmol, 1.0 eq), dichloromethane (10 mL), EDCI (533 mg, 2.8 mmol, 2.0 eq), N,N-dimethylpyridin-4-amine (679 mg, 5.6 mmol, 4.0 eq). The reaction mixture was stirred overnight at 30° C. The resulting mixture was concentrated under vacuum. The crude residue was applied onto a silica gel column and eluted with dichloromethane/ethyl acetate (included 20% of methanol)=1:1 to give 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide as a yellow solid (510 mg, 55.3%). ¹HNMR (300 MHz, DMSO-d₆) δ 12.27 (s, 1H), 8.70-8.53 (m, 2H), 7.92 (dd, J=9.3, 2.3 Hz, 1H), 7.36 (d, J=8.7 Hz, 1H), 7.27 (d, J=9.4 Hz, 1H), 7.11 (d, J=2.3 Hz, 1H), 6.95 (dd, J=8.9, 2.4 Hz, 1H), 4.24 (s, 1H), 3.33 (s, 7H), 2.84 (s, 4H), 1.81-1.61 (m, 7H), 1.55 (d, J=12.5 Hz, 1H), 1.43-0.98 (m, 7H).

Synthesis of N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into a 40 mL vial, were placed (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (210 mg, 0.6 mmol, 1.0 eq), 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (500 mg, 0.8 mmol, 1.3 eq), Dimethyl sulfoxide (10 mL), K₂CO₃ (241 mg, 1.7 mmol, 3.0 eq), CuI (55 mg, 0.3 mmol, 0.5 eq), N,N′-diphenyl-ethanediamide (70 mg, 0.3 mmol, 0.5 eq). The reaction mixture was stirred for 3 hours at 100° C. under nitrogen atmosphere. The resulting mixture was quenched by the addition of water (200 mL) and extracted with EtOAc (3×50 mL). The combined organic layer was washed with brine (200 mL), dried over anhydrous Na₂SO₄. After filtration, the filtrate was concentrated under vacuum. The crude residue was applied onto a silica gel column and eluted with dichloromethane/methanol=10:1 to give N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as a yellow solid (220 mg, 40.1%). LC-MS (ESI, m/z) M+1: 944.

### Example 1: Preparation of 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-[(oxan-4-ylmethyl)amino]benzenesulfonyl]benzamide

Synthesis of tert-butyl 2-[(2S)-2-[2-(prop-1-en-2-yl)phenyl]pyrrolidin-1-yl]-7-azaspiro[3.5]nonane-7-carboxylate: Into a 250-mL round-bottom flask purged and maintained with an inert atmosphere of nitrogen, were placed tert-butyl 2-[(2S)-2-(2-bromophenyl)pyrrolidin-1-yl]-7-azaspiro[3.5]nonane-7-carboxylate (7.2 g, 15.9 mmol, 1.0 eq), dioxane (72 mL), water (8 mL), 4,4,5,5-tetramethyl-2-(prop-1-en-2-yl)-1,3,2-dioxaborolane (6.4 g, 38.0 mmol, 2.4 eq), K₂CO₃ (6.6 g, 47.8 mmol, 3.0 eq), Pd(dqpf)Cl₂·CH₂Cl₂ (1.3 g, 1.6 mmol, 0.1 eq). The resulting solution was stirred 8 hours at 80° C. in an oil bath. The reaction mixture was cooled to 25° C. The resulting mixture was concentrated under vacuum. The crude residue was purified by a flash column (silica gel, ethyl acetate/petroleum ether=1: 2) to afford tert-butyl 2-[(2S)-2-[2-(prop-1-en-2-yl)phenyl]pyrrolidin-1-yl]-7-azaspiro[3.5]nonane-7-carboxylate (4 g, 66.4%) as yellow oil. LC-MS (ESI, m/z) M+1: 411.

Synthesis of tert-butyl 2-[(2S)-2-(2-isopropylphenyl)pyrrolidin-1-yl]-7-azaspiro[3.5]nonane-7-carboxylate: Into a 100-mL pressure tank reactor (10 atm), were placed tert-butyl 2-[(2S)-2-[2-(prop-1-en-2-yl)phenyl]pyrrolidin-1-yl]-7-azaspiro[3.5]nonane-7-carboxylate (3.6 g, 8.7 mmol, 1.0 eq), EtOH (50 mL), 10% Pd/C (500 mg, 4.7 mmol, 0.5 eq), to the above H₂ was introduced in. The resulting solution was stirred overnight at 50° C. in an oil bath. The reaction mixture was cooled to 25° C. and the solids were filtered out. The resulting mixture was concentrated under vacuum. The crude residue was purified by a flash column (silica gel, ethyl acetate/petroleum ether=1: 2) to afford tert-butyl 2-[(2S)-2-(2-isopropylphenyl)pyrrolidin-1-yl]-7-azaspiro[3.5]nonane-7-carboxylate (3 g, 72.3%) as yellow oil. LC-MS (ESI, m/z) M+1: 413.

Synthesis of 2-[(2S)-2-(2-isopropylphenyl)pyrrolidin-1-yl]-7-azaspiro[3.5]nonane hydrochloride: Into a 100-mL round-bottom flask, were placed tert-butyl 2-[(2S)-2-(2-isopropylphenyl)pyrrolidin-1-yl]-7-azaspiro[3.5]nonane-7-carboxylate (2.6 g, 6.3 mmol, 1.0 eq), HCl (gas) in 1,4-dioxane (20 mL). The resulting solution was stirred for 1 hour at 25° C. The resulting mixture was concentrated under vacuum. This resulted in 2-[(2S)-2-(2-isopropylphenyl)pyrrolidin-1-yl]-7-azaspiro[3.5]nonane hydrochloride (2 g, 95.5%) as a red solid. LC-MS (ESI, m/z) M+1: 313.

Synthesis of methyl 2-bromo-4-[2-[(2S)-2-(2-isopropylphenyl)pyrrolidin-1-yl]-7-azaspiro[3.5]nonan-7-yl]benzoate: Into a 100-mL round-bottom flask, were placed 2-[(2S)-2-(2-isopropylphenyl)pyrrolidin-1-yl]-7-azaspiro[3.5]nonane hydrochloride (1.3 g, 3.8 mmol, 1.0 eq), DMSO (20 mL), methyl 2-bromo-4-fluorobenzoate (1.7 g, 7.5 mmol, 2.0 eq), Na₂CO₃ (1.6 g, 15.2 mmol, 4.0 eq). The resulting solution was stirred for 16 hours at 110° C. in an oil bath. The reaction mixture was cooled to 25° C. The resulting solution was diluted with water (200 mL). The resulting solution was extracted with ethyl acetate (3×50 mL) and the organic layers combined. The resulting mixture was washed with brine (1×200 mL). The mixture was dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-[2-[(2S)-2-(2-isopropylphenyl)pyrrolidin-1-yl]-7-azaspiro[3.5]nonan-7-yl]benzoate (1 g, 52.8%) as yellow oil. LC-MS (ESI, m/z) M+1: 579/581. ¹HNMR (300 MHz, Chloroform-d) δ 7.80 (d, J=8.9 Hz, 1H), 7.64 (d, J=7.4 Hz, 1H), 7.27-7.14 (m, 3H), 7.06 (d, J=2.6 Hz, 1H), 6.73 (dd, J=9.0, 2.4 Hz, 1H), 3.87 (s, 3H), 3.68 (t, J=8.0 Hz, 1H), 3.34 (dq, J=13.7, 6.8, 6.1 Hz, 1H), 3.28-3.04 (m, 6H), 2.39 (q, J=8.8 Hz, 1H), 2.22 (dt, J=19.5, 8.2 Hz, 1H), 1.88 (dq, J=31.2, 10.5, 10.1 Hz, 4H), 1.75-1.42 (m, 6H), 1.26 (t, J=7.0 Hz, 6H).

Synthesis of methyl 4-[2-[(2S)-2-(2-isopropylphenyl)pyrrolidin-1-yl]-7-azaspiro[3.5]nonan-7-yl]-2-[(11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraen-10-yl]benzoate: Into a 40-mL vial purged and maintained with an inert atmosphere of nitrogen, were placed methyl 2-bromo-4-[2-[(2S)-2-(2-isopropylphenyl)pyrrolidin-1-yl]-7-azaspiro[3.5]nonan-7-yl]benzoate (770 mg, 1.5 mmol, 1.7 eq), toluene (10 mL), (11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene (300 mg, 0.8 mmol, 1.0 eq), Cs₂CO₃ (843 mg, 2.6 mmol, 3.0 eq), Xantphos (100 mg, 0.2 mmol, 0.2 eq), Pd₂(dba)₃·CHCl₃ (89 mg, 0.1 mmol, 0.1 eq). The resulting solution was stirred for 5 hours at 100° C. in an oil bath. The reaction mixture was cooled to 25° C. The resulting mixture was concentrated under vacuum. The crude residue was purified by a flash column (silica gel, ethyl acetate/petroleum ether=1:1) to afford methyl 4-[2-[(2S)-2-(2-isopropylphenyl)pyrrolidin-1-yl]-7-azaspiro[3.5]nonan-7-yl]-2-[(11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraen-10-yl]benzoate (600 mg, 87.7%) as a yellow solid. LC-MS (ESI, m/z) M+1: 792.

Synthesis of methyl 2-[(11R,15S)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoate: Into a 40-mL vial, were placed methyl 4-[2-[(2S)-2-(2-isopropylphenyl)pyrrolidin-1-yl]-7-azaspiro[3.5]nonan-7-yl]-2-[(11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (600 mg, 0.7 mmol, 1.0 eq), TBAF in THE (1.0 M, 10 mL), ethylenediamine (910 mg, 15.1 mmol, 20.0 eq). The resulting solution was stirred for 16 hours at 70° C. in an oil bath. The reaction mixture was cooled to 25° C. The resulting solution was diluted with water (200 mL). The resulting solution was extracted with of ethyl acetate (2×50 mL) and the organic layers combined. The resulting mixture was washed with brine (1×200 mL). The mixture was dried over anhydrous Na₂SO₄. After filtration, the filtrate was concentrated under reduced pressure. The crude residue was purified by a flash column (silica gel, ethyl acetate/petroleum ether=2: 1) to afford methyl 2-[(11R,15S)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoate (300 mg, 59.8%) as a yellow solid. LC-MS (ESI, m/z) M+1: 662.

Synthesis of 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid: Into a 100-mL round-bottom flask, were placed methyl 2-[(11R,15S)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoate (300 mg, 0.4 mmol, 1.0 eq), MeOH (5 mL), dioxane (5 mL), NaOH (2 mL). The resulting solution was stirred for 4 hours at 70° C. in an oil bath. The reaction mixture was cooled to 25° C. The resulting mixture was concentrated under vacuum. The pH value of the solution was adjusted to 6-7 with HCl (2 mol/L). The resulting solution was extracted with of ethyl acetate (2×50 mL) and the organic layers combined. The resulting mixture was washed with brine (1×200 mL). The mixture was dried over anhydrous Na₂SO₄. After filtration, the filtrate was concentrated under reduced pressure. The crude residue was purified by a flash column (silica gel, dichloromethane/methanol=10: 1) to afford 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid (240 mg, 81.7%) as a yellow solid. LC-MS (ESI, m/z) M+1: 648.

Synthesis of 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-[(oxan-4-ylmethyl)amino]benzenesulfonyl]benzamide: Into a 40-mL vial, were placed 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid (80 mg, 0.1 mmol, 1.0 eq), CH₂Cl₂ (5 mL), 3-nitro-4-[(oxan-4-ylmethyl)amino]benzenesulfonamide (39 mg, 0.1 mmol, 1.0 eq), EDCI (47 mg, 0.2 mmol, 2.0 eq), DMAP (60 mg, 0.5 mmol, 4.0 eq). The resulting solution was stirred overnight at 30° C. The resulting mixture was concentrated under vacuum. The crude residue was purified by a flash column (silica gel, dichloromethane/methanol=10:1). The crude product was purified by Prep-HPLC with the following conditions (2 #SHIMADZU (HPLC-01)): Column, XBridge Shield RP18 OBD Column, 19*150 mm, 5 um; mobile phase, water (0.05% NH₃·H₂O) and CH₃CN (44% Phase B up to 53% in 7 min); Detector, UV. This resulted in 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-[(oxan-4-ylmethyl)amino]benzenesulfonyl]benzamide (15 mg, 12.8%) as a yellow solid. LC-MS (ESI, m/z) M+1: 945. ¹HNMR (300 MHz, DMSO-d₆) δ 12.30 (s, 1H), 11.10 (d, J=55.7 Hz, 1H), 8.51 (s, 1H), 8.31 (d, J=2.3 Hz, 1H), 7.55 (s, 2H), 7.44-7.02 (m, 5H), 6.93-6.61 (m, 3H), 6.57 (s, 1H), 6.02 (ddd, J=13.3, 3.4, 1.8 Hz, 1H), 4.62 (s, 1H), 4.46 (q, J=8.4 Hz, 1H), 4.29 (s, 1H), 4.09 (t, J=7.2 Hz, 1H), 3.85 (t, J=11.4 Hz, 4H), 3.70-3.38 (m, 3H), 3.30-2.94 (m, 9H), 2.28 (d, J=2.0 Hz, 2H), 2.07-1.01 (m, 21H).

### Example 2: Preparation of 2-[(11S,15R)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-[(oxan-4-ylmethyl)amino]benzenesulfonyl]benzamide

Synthesis of tert-butyl 2-[(2S)-2-[2-(prop-1-en-2-yl)phenyl]pyrrolidin-1-yl]-7-azaspiro[3.5]nonane-7-carboxylate: Into a 250-mL round-bottom flask purged and maintained with an inert atmosphere of nitrogen, were placed tert-butyl 2-[(2S)-2-(2-bromophenyl)pyrrolidin-1-yl]-7-azaspiro[3.5]nonane-7-carboxylate (7.2 g, 15.9 mmol, 1.0 eq), dioxane (72 mL), water (8 mL), 4,4,5,5-tetramethyl-2-(prop-1-en-2-yl)-1,3,2-dioxaborolane (6.4 g, 38.0 mmol, 2.4 eq), K₂CO₃ (6.6 g, 47.8 mmol, 3.0 eq), Pd(dqpf)Cl₂·CH₂Cl₂ (1.3 g, 1.6 mmol, 0.1 eq). The resulting solution was stirred 8 hours at 80° C. in an oil bath. The reaction mixture was cooled to 25° C. The resulting mixture was concentrated under vacuum. The crude residue was purified by a flash column (silica gel, ethyl acetate/petroleum ether=1:2) to afford tert-butyl 2-[(2S)-2-[2-(prop-1-en-2-yl)phenyl]pyrrolidin-1-yl]-7-azaspiro[3.5]nonane-7-carboxylate (4 g, 66.4%) as yellow oil. LC-MS (ESI, m/z) M+1: 411.

Synthesis of tert-butyl 2-[(2S)-2-(2-isopropylphenyl)pyrrolidin-1-yl]-7-azaspiro[3.5]nonane-7-carboxylate: Into a 100-mL pressure tank reactor (10 atm), were placed tert-butyl 2-[(2S)-2-[2-(prop-1-en-2-yl)phenyl]pyrrolidin-1-yl]-7-azaspiro[3.5]nonane-7-carboxylate (3.6 g, 8.7 mmol, 1.0 eq), EtOH (50 mL), 10% Pd/C (500 mg, 4.7 mmol, 0.5 eq), to the above H₂ was introduced in. The resulting solution was stirred overnight at 50° C. in an oil bath. The reaction mixture was cooled to 25° C. and the solids were filtered out. The resulting mixture was concentrated under vacuum. The crude residue was purified by a flash column (silica gel, ethyl acetate/petroleum ether=1: 2) to afford tert-butyl 2-[(2S)-2-(2-isopropylphenyl)pyrrolidin-1-yl]-7-azaspiro[3.5]nonane-7-carboxylate (3 g, 72.3%) as yellow oil. LC-MS (ESI, m/z) M+1: 413.

Synthesis of 2-[(2S)-2-(2-isopropylphenyl)pyrrolidin-1-yl]-7-azaspiro[3.5]nonane hydrochloride: Into a 100-mL round-bottom flask, were placed tert-butyl 2-[(2S)-2-(2-isopropylphenyl)pyrrolidin-1-yl]-7-azaspiro[3.5]nonane-7-carboxylate (2.6 g, 6.3 mmol, 1.0 eq), HCl (gas) in 1,4-dioxane (20 mL). The resulting solution was stirred for 1 hour at 25° C. The resulting mixture was concentrated under vacuum. This resulted in 2-[(2S)-2-(2-isopropylphenyl)pyrrolidin-1-yl]-7-azaspiro[3.5]nonane hydrochloride (2 g, 95.5%) as a red solid. LC-MS (ESI, m/z) M+1: 313.

Synthesis of methyl 2-bromo-4-[2-[(2S)-2-(2-isopropylphenyl)pyrrolidin-1-yl]-7-azaspiro[3.5]nonan-7-yl]benzoate: Into a 100-mL round-bottom flask, were placed 2-[(2S)-2-(2-isopropylphenyl)pyrrolidin-1-yl]-7-azaspiro[3.5]nonane hydrochloride (1.3 g, 3.8 mmol, 1.0 eq), DMSO (20 mL), methyl 2-bromo-4-fluorobenzoate (1.7 g, 7.5 mmol, 2.0 eq), Na₂CO₃ (1.6 g, 15.2 mmol, 4.0 eq). The resulting solution was stirred for 16 hours at 110° C. in an oil bath. The reaction mixture was cooled to 25° C. The resulting solution was diluted with water (200 mL). The resulting solution was extracted with ethyl acetate (3×50 mL) and the organic layers combined. The resulting mixture was washed with brine (1×200 mL). The mixture was dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-[2-[(2S)-2-(2-isopropylphenyl)pyrrolidin-1-yl]-7-azaspiro[3.5]nonan-7-yl]benzoate (1 g, 52.8%) as yellow oil. LC-MS (ESI, m/z) M+1: 579/581. ¹HNMR (300 MHz, Chloroform-d) δ 7.80 (d, J=8.9 Hz, 1H), 7.64 (d, J=7.4 Hz, 1H), 7.27-7.14 (m, 3H), 7.06 (d, J=2.6 Hz, 1H), 6.73 (dd, J=9.0, 2.4 Hz, 1H), 3.87 (s, 3H), 3.68 (t, J=8.0 Hz, 1H), 3.34 (dq, J=13.7, 6.8, 6.1 Hz, 1H), 3.28-3.04 (m, 6H), 2.39 (q, J=8.8 Hz, 1H), 2.22 (dt, J=19.5, 8.2 Hz, 1H), 1.88 (dq, J=31.2, 10.5, 10.1 Hz, 4H), 1.75-1.42 (m, 6H), 1.26 (t, J=7.0 Hz, 6H).

Synthesis of methyl 4-[2-[(2S)-2-(2-isopropylphenyl)pyrrolidin-1-yl]-7-azaspiro[3.5]nonan-7-yl]. 2-[(11S,15R)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraen-10-yl]benzoate: Into a 40-mL vial purged and maintained with an inert atmosphere of nitrogen, were placed methyl 2-bromo-4-[2-[(2S)-2-(2-isopropylphenyl)pyrrolidin-1-yl]-7-azaspiro[3.5]nonan-7-yl]benzoate (257 mg, 0.5 mmol, 1.7 eq), toluene (5 mL), (11S,15R)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene (100 mg, 0.3 mmol, 1.0 eq), Cs₂CO₃ (281 mg, 0.8 mmol, 3.0 eq), Xantphos (33 mg, 0.1 mmol, 0.2 eq), Pd₂(dba)₃·CHCl₃ (30 mg, 0.1 mmol, 0.1 eq). The resulting solution was stirred for 5 hours at 100° C. in an oil bath. The reaction mixture was cooled to 25° C. The resulting mixture was concentrated under vacuum. The crude residue was purified by a flash column (silica gel, ethyl acetate/petroleum ether=1:1) to afford methyl 4-[2-[(2S)-2-(2-isopropylphenyl)pyrrolidin-1-yl]-7-azaspiro[3.5]nonan-7-yl]-2-[(11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (220 mg, 96.5%) as yellow oil. LC-MS (ESI, m/z) M+1: 792.

Synthesis of methyl 2-[(11S,15R)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoate: Into a 40-mL vial, were placed methyl 4-[2-[(2S)-2-(2-isopropylphenyl)pyrrolidin-1-yl]-7-azaspiro[3.5]nonan-7-yl]-2-[(11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (220 mg, 0.3 mmol, 1.0 eq), TBAF in THE (1.0 M, 10 mL), ethylenediamine (334 mg, 5.6 mmol, 20.0 eq). The resulting solution was stirred for 16 hours at 70° C. in an oil bath. The reaction mixture was cooled to 25° C. The resulting solution was diluted with water (200 mL). The resulting solution was extracted with of ethyl acetate (2×50 mL) and the organic layers combined. The resulting mixture was washed with brine (1×200 mL). The mixture was dried over anhydrous Na₂SO₄. After filtration, the filtrate was concentrated under reduced pressure. The crude residue was purified by a flash column (silica gel, ethyl acetate/petroleum ether=2: 1) to afford methyl 2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoate (100 mg, 54.4%) as a yellow solid. LC-MS (ESI, m/z) M+1: 662.

Synthesis of 2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid: Into a 40-mL vial, were placed methyl 2-[(11S,15R)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoate (100 mg, 0.2 mmol, 1.0 eq), MeOH (5 mL), dioxane (5 mL), NaOH (4 M, 2 mL). The resulting solution was stirred for 4 hours at 70° C. in an oil bath. The reaction mixture was cooled to 25° C. The resulting mixture was concentrated under vacuum. The pH value of the solution was adjusted to 6-7 with HCl (2.0 M). The resulting solution was extracted with of ethyl acetate (2×50 mL) and the organic layers combined. The resulting mixture was washed with brine (1×200 mL). The mixture was dried over anhydrous Na₂SO₄. After filtration, the filtrate was concentrated under reduced pressure. The crude residue was purified by a flash column (silica gel, dichloromethane/methanol=10: 1) to afford 2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid (90 mg, 91.9%) as a light yellow solid. LC-MS (ESI, m/z) M+1: 648.

Synthesis of 2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-[(oxan-4-ylmethyl)amino]benzenesulfonyl]benzamide: Into a 40-mL vial, were placed 2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid (90 mg, 0.1 mmol, 1.0 eq), CH₂Cl₂ (5 mL), 3-nitro-4-[(oxan-4-ylmethyl)amino]benzenesulfonamide (44 mg, 0.1 mmol, 1.0 eq), EDCI (53 mg, 0.2 mmol, 2.0 eq), DMAP (68 mg, 0.5 mmol, 4.0 eq). The resulting solution was stirred overnight at 30° C. The resulting mixture was concentrated under vacuum. The crude residue was purified by a flash column (silica gel, dichloromethane/methanol=10: 1). The crude product was purified by Prep-HPLC with the following conditions (2 #SHIMADZU (HPLC-01)): Column, XBridge Shield RP18 OBD Column, 19*150 mm, 5 um; mobile phase, water (0.05% NH₃·H₂O) and CH₃CN (44% Phase B up to 54% in 7 min); Detector, UV. This resulted in 2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-[(oxan-4-ylmethyl)amino]benzenesulfonyl]benzamide (40 mg, 30.4%) as a yellow solid. LC-MS (ESI, m/z) M+1: 945. ¹HNMR (300 MHz, DMSO-d₆) δ 12.30 (s, 1H), 11.10 (d, J=55.7 Hz, 1H), 8.51 (s, 1H), 8.31 (d, J=2.3 Hz, 1H), 7.55 (s, 2H), 7.44-7.02 (m, 5H), 6.93-6.61 (m, 3H), 6.57 (s, 1H), 6.02 (ddd, J=13.3, 3.4, 1.8 Hz, 1H), 4.62 (s, 1H), 4.46 (q, J=8.4 Hz, 1H), 4.29 (s, 1H), 4.09 (t, J=7.2 Hz, 1H), 3.85 (t, J=11.4 Hz, 4H), 3.70-3.38 (m, 3H), 3.30-2.94 (m, 9H), 2.28 (d, J=2.0 Hz, 2H), 2.07-1.01 (m, 21H).

### Example 3: Preparation of 2-[(11R,15S)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-([[(1r,4r)-4-hydroxy-4-methylcyclohexyl]methyl]amino)benzenesulfonyl]benzamide and 2-[(11R,15S)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-([[(1s,4s)-4-hydroxy-4-methylcyclohexyl]methyl]amino)benzenesulfonyl]benzamide (assumed)

Synthesis of tert-butyl 2-[(2S)-2-[2-(prop-1-en-2-yl)phenyl]pyrrolidin-1-yl]-7-azaspiro[3.5]nonane-7-carboxylate: Into a 250-mL round-bottom flask purged and maintained with an inert atmosphere of nitrogen, was placed tert-butyl 2-[(2S)-2-(2-bromophenyl)pyrrolidin-1-yl]-7-azaspiro[3.5]nonane-7-carboxylate (7.2 g, 15.9 mmol, 1.0 eq), dioxane (72 mL), water (8 mL), 4,4,5,5-tetramethyl-2-(prop-1-en-2-yl)-1,3,2-dioxaborolane (6.4 g, 38.0 mmol, 2.4 eq), K₂CO₃ (6.6 g, 47.8 mmol, 3.0 eq), Pd(dppf)Cl₂·CH₂Cl₂ (1.3 g, 1.6 mmol, 0.1 eq). The resulting solution was stirred 8 hours at 80° C. in an oil bath. The reaction mixture was cooled to 25° C. The resulting mixture was concentrated under vacuum. The crude residue was purified by a flash column (silica gel, ethyl acetate/petroleum ether=1:2) to afford tert-butyl 2-[(2S)-2-[2-(prop-1-en-2-yl)phenyl]pyrrolidin-1-yl]-7-azaspiro[3.5]nonane-7-carboxylate (4 g, 66.4%) as yellow oil. LC-MS (ESI, m/z) M+1: 411.

Synthesis of tert-butyl 2-[(2S)-2-(2-isopropylphenyl)pyrrolidin-1-yl]-7-azaspiro[3.5]nonane-7-carboxylate: Into a 100-mL pressure tank reactor (10 atm), was placed tert-butyl 2-[(2S)-2-[2-(prop-1-en-2-yl)phenyl]pyrrolidin-1-yl]-7-azaspiro[3.5]nonane-7-carboxylate (3.6 g, 8.7 mmol, 1.0 eq), EtOH (50 mL), 10% Pd/C (500 mg, 4.7 mmol, 0.5 eq), to the above H₂ was introduced in. The resulting solution was stirred overnight at 50° C. in an oil bath. The reaction mixture was cooled to 25° C. and the solids were filtered out. The resulting mixture was concentrated under vacuum. The crude residue was purified by a flash column (silica gel, ethyl acetate/petroleum ether=1:2) to afford tert-butyl 2-[(2S)-2-(2-isopropylphenyl)pyrrolidin-1-yl]-7-azaspiro[3.5]nonane-7-carboxylate (3 g, 72.3%) as yellow oil. LC-MS (ESI, m/z) M+1: 413.

Synthesis of 2-[(2S)-2-(2-isopropylphenyl)pyrrolidin-1-yl]-7-azaspiro[3.5]nonane hydrochloride: Into a 100-mL round-bottom flask, was placed tert-butyl 2-[(2S)-2-(2-isopropylphenyl)pyrrolidin-1-yl]-7-azaspiro[3.5]nonane-7-carboxylate (2.6 g, 6.3 mmol, 1.0 eq), HCl (gas) in 1,4-dioxane (20 mL). The resulting solution was stirred for 1 hour at 25° C. The resulting mixture was concentrated under vacuum. This resulted in 2-[(2S)-2-(2-isopropylphenyl)pyrrolidin-1-yl]-7-azaspiro[3.5]nonane hydrochloride (2 g, 95.5%) as a red solid. LC-MS (ESI, m/z) M+1: 313.

Synthesis of methyl 2-bromo-4-[2-[(2S)-2-(2-isopropylphenyl)pyrrolidin-1-yl]-7-azaspiro[3.5]nonan-7-yl]benzoate: Into a 100-mL round-bottom flask, was placed 2-[(2S)-2-(2-isopropylphenyl)pyrrolidin-1-yl]-7-azaspiro[3.5]nonane hydrochloride (1.3 g, 3.8 mmol, 1.0 eq), DMSO (20 mL), methyl 2-bromo-4-fluorobenzoate (1.7 g, 7.5 mmol, 2.0 eq), Na₂CO₃ (1.6 g, 15.2 mmol, 4.0 eq). The resulting solution was stirred for 16 hours at 110° C. in an oil bath. The reaction mixture was cooled to 25° C. The resulting solution was diluted with water (200 mL). The resulting solution was extracted with ethyl acetate (3×50 mL) and the organic layers combined. The resulting mixture was washed with brine (1×200 mL). The mixture was dried over anhydrous Na₂SO₄. After filtration, the filtrate was concentrated under reduced pressure. The crude residue was purified by a flash column (silica gel, ethyl acetate/petroleum ether=1:2) to afford methyl 2-bromo-4-[2-[(2S)-2-(2-isopropylphenyl)pyrrolidin-1-yl]-7-azaspiro[3.5]nonan-7-yl]benzoate (1 g, 52.8%) as yellow oil. LC-MS (ESI, m/z) M+1: 579. ¹HNMR (300 MHz, Chloroform-d) δ 7.80 (d, J=8.9 Hz, 1H), 7.64 (d, J=7.4 Hz, 1H), 7.27-7.14 (m, 3H), 7.06 (d, J=2.6 Hz, 1H), 6.73 (dd, J=9.0, 2.4 Hz, 1H), 3.87 (s, 3H), 3.68 (t, J=8.0 Hz, 1H), 3.34 (dq, J=13.7, 6.8, 6.1 Hz, 1H), 3.28-3.04 (m, 6H), 2.39 (q, J=8.8 Hz, 1H), 2.22 (dt, J=19.5, 8.2 Hz, 1H), 1.88 (dq, J=31.2, 10.5, 10.1 Hz, 4H), 1.75-1.42 (m, 6H), 1.26 (t, J=7.0 Hz, 6H).

Synthesis of methyl 4-[2-[(2S)-2-(2-isopropylphenyl)pyrrolidin-1-yl]-7-azaspiro[3.5]nonan-7-yl]-2-[(11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraen-10-yl]benzoate: Into a 40-mL vial purged and maintained with an inert atmosphere of nitrogen, was placed methyl 2-bromo-4-[2-[(2S)-2-(2-isopropylphenyl)pyrrolidin-1-yl]-7-azaspiro[3.5]nonan-7-yl]benzoate (770 mg, 1.5 mmol, 1.7 eq), toluene (10 mL), (11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene (300 mg, 0.8 mmol, 1.0 eq), Cs₂CO₃ (843 mg, 2.6 mmol, 3.0 eq), Xantphos (100 mg, 0.2 mmol, 0.2 eq), Pd₂(dba)₃·CHCl₃ (89 mg, 0.1 mmol, 0.1 eq). The resulting solution was stirred for 5 hours at 100° C. in an oil bath. The reaction mixture was cooled to 25° C. The resulting mixture was concentrated under vacuum. The crude residue was purified by a flash column (silica gel, ethyl acetate/petroleum ether=1:1) to afford methyl 4-[2-[(2S)-2-(2-isopropylphenyl)pyrrolidin-1-yl]-7-azaspiro[3.5]nonan-7-yl]-2-[(11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraen-10-yl]benzoate (600 mg, 87.7%) as a yellow solid. LC-MS (ESI, m/z) M+1: 792.

Synthesis of methyl 2-[(11R,15S)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoate: Into a 40-mL vial, was placed methyl 4-[2-[(2S)-2-(2-isopropylphenyl)pyrrolidin-1-yl]-7-azaspiro[3.5]nonan-7-yl]-2-[(11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (600 mg, 0.7 mmol, 1.0 eq), TBAF in THE (1.0 M, 10 mL), ethylenediamine (910 mg, 15.1 mmol, 20.0 eq). The resulting solution was stirred for 16 hours at 70° C. in an oil bath. The reaction mixture was cooled to 25° C. The resulting solution was diluted with water (200 mL). The resulting solution was extracted with of ethyl acetate (2×50 mL) and the organic layers combined. The resulting mixture was washed with brine (1×200 mL). The mixture was dried over anhydrous Na₂SO₄. After filtration, the filtrate was concentrated under reduced pressure. The crude residue was purified by a flash column (silica gel, ethyl acetate/petroleum ether=2:1) to afford methyl 2-[(11R,15S)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoate (300 mg, 59.8%) as a yellow solid. LC-MS (ESI, m/z) M+1: 662.

Synthesis of 2-[(11R,15S)-13,16-dioxa-2,4,10-triazatetracyclo[7.7.0.0\hat{ }[3,7].0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid: Into a 100-mL round-bottom flask, was placed methyl 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoate (300 mg, 0.4 mmol, 1.0 eq), MeOH (5 mL), dioxane (5 mL), NaOH (2 mL). The resulting solution was stirred for 4 hours at 70° C. in an oil bath. The reaction mixture was cooled to 25° C. The resulting mixture was concentrated under vacuum. The pH value of the solution was adjusted to 6-7 with HCl (2.0 M). The resulting solution was extracted with of ethyl acetate (2×50 mL) and the organic layers combined. The resulting mixture was washed with brine (1×200 mL). The mixture was dried over anhydrous Na₂SO₄. After filtration, the filtrate was concentrated under reduced pressure. The crude residue was purified by a flash column (silica gel, dichloromethane/methanol=10:1) to afford 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid (240 mg, 81.7%) as a yellow solid. LC. MS (ESI, m/z) M+1: 648.

Synthesis of 8-methyl-1,4-dioxaspiro[4.5]decan-8-ol: Into a 1-L 3-necked round-bottom flask purged and maintained with an inert atmosphere of nitrogen, was placed 1,4-dioxaspiro[4.5]decan-8-one (100.0 g, 640.3 mmol), THE (500 mL). This was followed by the addition of CH₃MgBr (256 mL, 3.0 M), in portions at 0° C. The resulting solution was stirred for 4 hours at 0° C. The reaction was then quenched by the addition of aqueous NH₄Cl. The resulting mixture was concentrated. The residue was dissolved in CH₂Cl₂ (500 mL). The resulting mixture was washed with water (3×50 mL). The mixture was dried over anhydrous sodium sulfate. After filtration, the filtrate was concentrated under reduced pressure. This resulted in 8-methyl-1,4-dioxaspiro[4.5]decan-8-ol (110 g, 99.7%) as white oil. ¹HNMR (300 MHz, Chloroform-d) δ: 3.98-3.93 (m, 4H), 2.07-1.52 (m, 8H), 1.27 (s, 3H).

Synthesis of 4-hydroxy-4-methylcyclohexan-1-one: Into a 1-L round-bottom flask, was placed 8-methyl-1,4-dioxaspiro[4.5]decan-8-ol (110.0 g, 639.5 mmol, 1.0 eq), 0.05 N HCl (500 mL). The resulting solution was stirred for 4 hours at 70° C. in an oil bath. The mixture was allowed to cool down to 25° C. The resulting mixture was extracted with Ethyl acetate (3×300 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1:2) to afford 4-hydroxy-4-methylcyclohexan-1-one (40 g, 48.9%) as yellow oil. ¹HNMR (300 MHz, Chloroform-d) δ 2.73 (ddd, J=15.3, 12.9, 6.3 Hz, 2H), 2.33-2.18 (m, 2H), 2.06-1.93 (m, 2H), 1.93-1.78 (m, 2H), 1.38 (s, 3H).

Synthesis of 1-methyl-4-(nitromethyl)cyclohex-3-en-1-ol: Into a 500-mL round-bottom flask, was placed 4-hydroxy-4-methylcyclohexan-1-one (20.0 g, 156.0 mmol, 1.0 eq), CH₃NO₂ (100 mL), methyl[2-(methylamino)ethyl]amine (1.4 g, 15.6 mmol, 0.1 eq). The resulting solution was stirred for 6 hours at 100° C. in an oil bath. The mixture was allowed to cool down to 25° C. The resulting mixture was concentrated. The crude residue was purified by a flash column (silica gel, ethyl acetate/petroleum ether=1:3) to afford 1-methyl-4-(nitromethyl)cyclohex-3-en-1-ol (21 g, 78.6%) as brown oil. ¹HNMR (300 MHz, Chloroform-d) δ 5.87 (t, J=2.7 Hz, 1H), 4.87 (s, 2H), 2.41-2.11 (m, 4H), 1.85-1.62 (m, 2H), 1.54 (d, J=16.0 Hz, 1H), 1.31 (s, 3H).

Synthesis of 4-(aminomethyl)-1-methylcyclohexan-1-ol: Into a 100-mL pressure tank reactor (20 atm), was placed 1-methyl-4-(nitromethyl)cyclohex-3-en-1-ol (5.0 g, 29.2 mmol, 1.0 eq), i-PrOH (50 mL), Pt/C (1.1 g, 5.8 mmol). To the above, H₂ (gas) was introduced in. The resulting solution was stirred for 20 hours at 30° C. in an oil bath. The solids were filtered out. The resulting mixture was concentrated. This resulted in 4-(aminomethyl)-1-methylcyclohexan-1-ol (2.5 g, crude) as black oil.

Synthesis of 3-nitro-4-([[(1r,4r)-4-hydroxy-4-methylcyclohexyl]methyl]amino)benzenesulfonamide and 3-nitro-4-([[(1s,4s)-4-hydroxy-4-methylcyclohexyl]methyl]amino)benzenesulfonamide: Into a 100-mL round-bottom flask, was placed 4-(aminomethyl)-1-methylcyclohexan-1-ol (2.5 g, crude), 4-fluoro-3-nitrobenzenesulfonamide (3.8 g, 17.4 mmol, 1.0 eq), THE (30 mL), TEA (5.3 g, 52.4 mmol, 3.0 eq). The resulting solution was stirred for 14 hours at 25° C. The resulting mixture was concentrated. The residue was applied onto a C18 gel column with (water:CH₃CN=100:0 to 100:30). This resulted in 1 g of 3-nitro-4-([[(1r,4r)-4-hydroxy-4-methylcyclohexyl]methyl]amino)benzenesulfonamide as a yellow solid. This resulted in 500 mg of 3-nitro-4-([[(1s,4s)-4-hydroxy-4-methylcyclohexyl]methyl]amino)benzenesulfonamide as a yellow solid. ¹HNMR-P1 (400 MHz, DMSO-d₆) δ 8.51 (d, J=6.4 Hz, 1H), 8.47 (s, 1H), 7.85 (d, J=9.2 Hz, 1H), 7.36 (s, 2H), 7.27 (dd, J=9.2, 2.5 Hz, 1H), 4.26 (d, J=2.8 Hz, 1H), 3.34 (s, 1H), 2.11 (d, J=17.6 Hz, OH), 1.69 (d, J=13.6 Hz, 3H), 1.55 (d, J=12.8 Hz, 2H), 1.34 (t, J=12.8 Hz, 2H), 1.16 (d, J=12.8 Hz, 1H), 1.10-1.06 (m, 4H). ¹HNMR-P2 (400 MHz, DMSO-d₆) δ 8.54 (d, J=6.4 Hz, 1H), 8.47 (s, 1H), 7.84 (d, J=9.2 Hz, 1H), 7.34 (s, 2H), 7.26 (d, J=9.2 Hz, 1H), 4.24-4.08 (m, 1H), 3.96 (s, 1H), 3.32 (d, J=6.4 Hz, 2H), 3.20-3.11 (m, 1H), 1.57 (s, 2H), 1.48 (d, J=12.4 Hz, 3H), 1.40 (d, J=12.4 Hz, 2H), 1.23 (td, J=13.2, 3.6 Hz, 3H), 1.08 (s, 3H).

Synthesis of 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-([[(1r,4r)-4-hydroxy-4-methylcyclohexyl]methyl]amino)benzenesulfonyl]benzamide Into a 40-mL vial, was placed 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid (80 mg, 0.1 mmol, 1.0 eq), CH₂Cl₂ (5 mL), 3-nitro-4-([[(1r,4r)-4-hydroxy-4-methylcyclohexyl]methyl]amino)benzenesulfonamide (42 mg, 0.1 mmol, 1.0 eq), EDCI (47 mg, 0.2 mmol, 2.0 eq), DMAP (60 mg, 0.5 mmol, 4.0 eq). The resulting solution was stirred overnight at 30° C. The resulting mixture was concentrated under vacuum. The crude residue was purified by a flash column (silica gel, dichloromethane/methanol=10:1). The crude product was purified by Prep-HPLC with the following conditions (2 #SHIMADZU (HPLC-01)): Column, XBridge Shield RP18 OBD Column, 19*150 mm, 5 um; mobile phase, water (0.05% NH₃·H₂O) and CH₃CN (44% Phase CH₃CN up to 54% in 7 min); Detector, UV. This resulted in 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-([[(1r,4r)-4-hydroxy-4-methylcyclohexyl]methyl]amino)benzenesulfonyl]benzamide (28 mg, 23.3%) as a yellow solid. LC-MS (ESI, m/z) M+1: 973. ¹HNMR (300 MHz, DMSO-d₆) δ 8.61-8.21 (m, 2H), 7.55 (s, 2H), 7.41-6.96 (m, 5H), 6.71 (dd, J=62.8, 25.1 Hz, 4H), 6.01 (ddd, J=13.5, 3.4, 1.9 Hz, 1H), 4.65 (s, 1H), 4.54-4.39 (m, 1H), 4.24 (s, 2H), 4.08 (d, J=7.0 Hz, 1H), 3.83 (dd, J=18.7, 9.5 Hz, 1H), 3.50 (dt, J=35.3, 8.8 Hz, 5H), 3.31-2.88 (m, 9H), 2.44-2.02 (m, 2H), 2.04-1.29 (m, 17H), 1.27-1.02 (m, 11H).

Synthesis of 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-([[(1s,4s)-4-hydroxy-4-methylcyclohexyl]methyl]amino)benzenesulfonyl]benzamide Into a 40-mL vial, was placed 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]benzoic acid (80 mg, 0.1 mmol, 1.0 eq), CH₂Cl₂ (5 mL), 3-nitro-4-([[(1s,4s)-4-hydroxy-4-methylcyclohexyl]methyl]amino)benzenesulfonamide (42 mg, 0.1 mmol, 1.0 eq), EDCI (47 mg, 0.2 mmol, 2.0 eq), DMAP (60 mg, 0.5 mmol, 4.0 eq). The resulting solution was stirred overnight at 30° C. The resulting mixture was concentrated under vacuum. The crude residue was purified by a flash column (silica gel, dichloromethane/methanol=10:1). The crude product was purified by Prep-HPLC with the following conditions (2 #SHIMADZU (HPLC-01)): Column, XBridge Shield RP18 OBD Column, 19*150 mm, 5 um; mobile phase, water (0.05% NH₃·H₂O) and CH₃CN (44% Phase CH₃CN up to 54% in 7 min); Detector, UV. This resulted in 2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-4-[2-[(2S)-2-(2-isopropylphenyl)pyrrolidin-1-yl]-7-azaspiro[3.5]nonan-7-yl]-N-[3-nitro-4-([[(1s,4s)-4-hydroxy-4-methylcyclohexyl]methyl]amino)benzenesulfonyl]benzamide (15 mg, 12.4%) as a yellow solid. LC-MS (ESI, m/z) M+1: 973. ¹HNMR (300 MHz, DMSO-d₆) δ 8.61-8.21 (m, 2H), 7.55 (s, 2H), 7.41-6.96 (m, 5H), 6.71 (dd, J=62.8, 25.1 Hz, 4H), 6.01 (ddd, J=13.5, 3.4, 1.9 Hz, 1H), 4.65 (s, 1H), 4.54-4.39 (m, 1H), 4.24 (s, 2H), 4.08 (d, J=7.0 Hz, 1H), 3.83 (dd, J=18.7, 9.5 Hz, 1H), 3.50 (dt, J=35.3, 8.8 Hz, 5H), 3.31-2.88 (m, 9H), 2.44-2.02 (m, 2H), 2.04-1.29 (m, 17H), 1.27-1.02 (m, 11H).

### Example 4: Preparation of Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of (S)-2-methyl-N-[(2-methylphenyl)methylidene]propane-2-sulfinamide: Into a 500 mL 3-necked round-bottom flask were added 2-methylbenzaldehyde (20.0 g, 166.4 mmol, 1.0 eq) and (S)-2-methylpropane-2-sulfinamide (24.2 g, 199.7 mmol, 1.2 eq), tetrahydrofuran (200 mL), Ti(OEt)₄ (56.9 g, 249.6 mmol, 1.5 eq) at 25° C. The resulting mixture was stirred for overnight at 55° C. The reaction was quenched by the addition of water (300 mL) at 25° C. The resulting mixture was extracted with Ethyl acetate (3×200 mL). The combined organic layers were washed with brine (1×300 mL), dried over anhydrous Na₂SO₄. After filtration, the filtrate was concentrated under reduced pressure. This crude resulted in (S)-2-methyl-N-[(2-methylphenyl)methylidene]propane-2-sulfinamide (30 g, 80.7%) as light yellow oil. LC-MS (ESI, m/z) M+1: 224. ¹HNMR (300 MHz, DMSO-d₆) δ 8.73 (s, 1H), 7.88 (dd, J=7.7, 1.6 Hz, 1H), 7.45 (td, J=7.4, 1.5 Hz, 1H), 7.43-7.25 (m, 2H), 2.54 (s, 3H), 1.17 (s, 9H).

Synthesis of (S)—N-[(1S)-3-(1,3-dioxan-2-yl)-1-(2-methylphenyl)propyl]-2-methylpropane-2-sulfinamide: Into a 1000 mL 3-necked round-bottom flask were added magnesium (5.4 g, 223.8 mmol, 2.0 eq) and iodine (60 mg, 0.2 mmol, 0.002 eq), tetrahydrofuran (20 mL) at 25° C. 2-(2-bromoethyl)-1,3-dioxane (43.6 g, 223.8 mmol, 2.0 eq) in tetrahydrofuran (220 mL) was added dropwise at 25° C. under nitrogen atmosphere. The resulting mixture was stirred for 30 mins at 25° C. To the above mixture was added (S)-2-methyl-N-[(2-methylphenyl)methylidene]propane-2-sulfinamide (25 g, 111.942 mmol, 1.00 eq) in CH₂Cl₂(100 mL) dropwise 10 mins at −40° C. The resulting mixture was stirred for additional 5 hours at −40° C. The reaction was quenched with sat.NH₄Cl (aq.) at 25° C. The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=10: 1) to afford (S)—N-[(1S)-3-(1,3-dioxan-2-yl)-1-(2-methylphenyl)propyl]-2-methylpropane-2-sulfinamide (35 g, 92.1%) as light yellow oil. LC-MS (ESI, m/z) M+1: 340.

Synthesis of (2S)-2-(2-methylphenyl)pyrrolidine: Into a 500 mL 3-necked round-bottom flask were added (S)—N-[(1S)-3-(1,3-dioxan-2-yl)-1-(2-methylphenyl)propyl]-2-methylpropane-2-sulfinamide (35.0 g, 103.1 mmol, 1.0 eq) and Trifluoroacetic acid (20 mL), water (18 mL) at 25° C. The resulting mixture was stirred for 1 hour at 25° C. To the above mixture was added Trifluoroacetic acid (210 mL), TESiH (40.2 g, 309.3 mmol, 3.0 eq). The resulting mixture was stirred for additional 20 hours at 25° C. The resulting mixture was concentrated under reduced pressure. The resulting mixture was diluted with water (300 mL). The aqueous layer was extracted with methyl tert-butyl ether (300 mL). The aqueous layer was basified to pH to 13 with 1 M NaOH. The resulting mixture was extracted with CH₂Cl₂ (2×300 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=10: 1 to 1:1) to afford (2S)-2-(2-methylphenyl)pyrrolidine (15 g) crude(ee=87%). The crude residue was dissolved in ethanol/ethyl acetate (35:65, 300 mL). To the above mixture was added dibenzoyl-L-taratric acid at 25° C. The resulting mixture was stirred for additional 10 mins at 90° C. The mixture was allowed to cool down to 25° C. The precipitated solids were collected by filtration and washed with Ethyl acetate (20 mL). The filter cake was collected and the crude residue was dissolved in water (100 mL). The mixture was acidified to pH 10 with 1 M NaOH. The resulting mixture was extracted with CH₂Cl₂ (3×200 mL). The combined organic layers dried over anhydrous Na₂SO₄. After filtration, the filtrate was concentrated under reduced pressure. This crude resulted in (2S)-2-(2-methylphenyl)pyrrolidine (6.5 g, 39.1%) (ee=98.6%) as yellow oil. LC-MS (ESI, m/z) M+1:162. ¹HNMR (300 MHz, DMSO-d₆) δ 7.52 (dd, J=7.2, 1.5 Hz, 1H), 7.19-7.01 (m, 3H), 4.19 (t, J=7.5 Hz, 1H), 3.04 (dd, J=9.7, 7.1, 5.4 Hz, 1H), 2.89 (dt, J=9.7, 7.5 Hz, 1H), 2.56 (s, 1H), 2.29 (s, 3H), 2.14 (dtd, J=12.1, 7.7, 5.8 Hz, 1H), 1.74 (dd, J=12.3, 9.8 Hz, 2H), 1.41-1.13 (m, 1H).

Synthesis of 7-azaspiro[3.5]nonan-2-one hydrochloride: Into a 500 mL 3-necked round-bottom flask were added te25-butyl 2-oxo-7-azaspiro[3.5]nonane-7-carboxylate (10 g, 41.8 mmol, 1.0 eq), Ethyl acetate (20 mL) and 2.0 M HCl in ethyl acetate (80 mL) at 25° C. The resulting mixture was stirred for 4 hours at 25° C. The resulting mixture was concentrated under reduced pressure. This crude resulted in 7-azaspiro[3.5]nonan-2-one hydrochloride (7.3 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (20 mL). The resulting mixture was extracted with ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=10: 1-2:1) to afford methyl 2-bromo-4-{2-oxo-7-azaspiro[3.5]nonan-7-yl}benzoate (6.8 g, 46.4%) as light yellow solid. LC-MS (ESI, m/z) M+1:352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-[(2S)-2-(2-methylphenyl)pyrrolidin-1-yl]-7-azaspiro[3.5]nonan. 7-yl}benzoate: Into a 100 mL round-bottom flask were added methyl 2-bromo-4-{2-oxo-7-azaspiro[3.5]nonan-7-yl}benzoate (500 mg, 1.4 mmol, 1.0 eq), 1,2-dichloroethane (5 mL) and (2S)-2-(2-methylphenyl)pyrrolidine (229 mg, 1.4 mmol, 1.0 eq) at 25° C. The resulting mixture was stirred for 30 mins at 25° C. To the above mixture was added NaHB(OAc)₃ (602 mg, 2.8 mmol, 2.0 eq) in portions at 25° C. The resulting mixture was stirred for additional 4 hours at 25° C. The reaction was quenched by the addition of MeOH (10 mL) at 25° C. The resulting mixture was concentrated under reduced pressure. The resulting mixture was concentrated under reduced pressure. The crude residue was purified by a flash column (silica gel, ethyl acetate/petroleum ether=10: 1 to 1: 1) to afford methyl 2-bromo-4-{2-[(2S)-2-(2-methylphenyl)pyrrolidin-1-yl]-7-azaspiro[3.5]nonan-7-yl}benzoate (500 mg, 70.8%) as light yellow solid. LC-MS (ESI, m/z) M+1: 497.

Synthesis of 2-bromo-4-{2-[(2S)-2-(2-methylphenyl)pyrrolidin-1-yl]-7-azaspiro[3.5]nonan-7-yl}benzoic acid: Into a 20 mL vial were added methyl 2-bromo-4-{2-[(2S)-2-(2-methylphenyl)pyrrolidin-1-yl]-7-azaspiro[3.5]nonan-7-yl}benzoate (500 mg, 1.0 mmol, 1.0 eq), MeOH (2.5 mL), water (2.5 mL) and NaOH (160 mg, 4.0 mmol, 4.0 eq) at 25° C. The resulting mixture was stirred for 4 hours at 40° C. The mixture was allowed to cool down to 25° C. The resulting mixture was diluted with water (10 mL). The mixture residue was acidified to pH 5 with 2.0 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. This crude resulted in 2-bromo-4-{2-[(2S)-2-(2-methylphenyl)pyrrolidin-1-yl]-7-azaspiro[3.5]nonan-7-yl}benzoic acid (190 mg, 39.1%) as light yellow solid. LC-MS (ESI, m/z) M+1: 483.

Synthesis of 2-bromo-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide: Into a 8 mL vial were added 2-bromo-4-{2-[(2S)-2-(2-methylphenyl)pyrrolidin-1-yl]-7-azaspiro[3.5]nonan-7-yl}benzoic acid (190 mg, 0.4 mmol, 1.0 eq), 3-nitro-4-[(oxan-4-ylmethyl)amino]benzenesulfonamide (123 mg, 0.4 mmol, 1.0 eq), DMAP (95 mg, 0.8 mmol, 2.0 eq), EDCI (90 mg, 0.5 mmol, 1.2 eq) in CH₂Cl₂ (3 mL) at 25° C. The resulting mixture was stirred for 3 hours at 30° C. The resulting mixture was diluted with water (10 mL). The resulting mixture was extracted with CH₂Cl₂ (3×5 mL). The combined organic layers were washed with brine (1×10 mL), dried over anhydrous Na₂SO₄. After filtration, the filtrate was concentrated under reduced pressure. The crude residue was purified by a flash column (silica gel, CH₂Cl₂/MeOH=10: 1) to afford 2-bromo-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide (200 mg, 65.1%) as light yellow solid. LC-MS (ESI, m/z) M+1: 780.

Synthesis of 4-{2-[(2S)-2-(2-methylphenyl)pyrrolidin-1-yl]-7-azaspiro[3.5]nonan-7-yl}-N-{3-nitro. 4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), caesio methaneperoxoate caesium (617 mg, 1.8 mmol, 2 eq), copper(I) iodide (36 mg, 0.18 mmol, 0.2 eq), N₁,N₂-diphenyloxalamide (45 mg, 0.18 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, PE/(EA/DCM=1:1)=(10: 1-1: 10) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 46.9%) as yellow solid. LC-MS (ESI, m/z) M+1: 1047.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide: Into a 8 mL vial were added 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (27 mg, 0.03 mmol, 1.0 eq), 1M TBAF in tetrahydrofuran (0.5 mL) and ethylenediamine (23 mg, 0.4 mmol, 15 eq) at 25° C. The resulting mixture was stirred for 2 hours at 70° C. The mixture was allowed to cool down to 25° C. The resulting mixture was diluted with water (5 mL). The resulting mixture was extracted with Ethyl acetate (3×5 mL). The combined organic layers were washed with brine (1×5 mL), dried over anhydrous Na₂SO₄. After filtration, the filtrate was concentrated under reduced pressure. The crude residue was purified by reverse flash chromatography with the following conditions: Column, XBridge Shield RP18 OBD Column, 19*150 mm, 5 μm; mobile phase, H₂O (10 mmol/L NH₄HCO₃ and 0.1% NH₃·H₂O) and CH₃CN (37% CH₃CN up to 55% in 7 min); UV 254 nm. This crude resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide (20 mg, 84.6%) as light yellow solid. LC-MS (ESI, m/z) M+1: 917. ¹HNMR (300 MHz, DMSO-d₆) δ 11.01 (s, 1H), 8.32 (d, J=2.3 Hz, 1H), 8.28-8.14 (m, 1H), 7.59 (d, J=8.7 Hz, 1H), 7.50 (d, J=7.6 Hz, 1H), 7.47-7.39 (m, 1H), 7.36 (d, J=8.6 Hz, 1H), 7.17-7.10 (m, 1H), 7.13-6.95 (m, 3H), 6.74 (s, 1H), 6.65 (d, J=9.1 Hz, 1H), 6.61 (d, J=2.6 Hz, 1H), 6.49 (s, 1H), 6.20 (d, J=9.1 Hz, 1H), 6.04 (d, J=2.8 Hz, 1H), 5.98 (d, J=2.5 Hz, 1H), 4.75 (d, J=8.6 Hz, 1H), 4.53-4.35 (m, 1H), 4.17-4.05 (m, 1H), 4.02-3.92 (m, 1H), 3.87 (dd, J=11.5, 4.4 Hz, 3H), 3.73 (t, J=5.0 Hz, 1H), 3.65 (t, J=7.0 Hz, 1H), 3.59-3.48 (m, 1H), 3.16-3.01 (m, 6H), 2.95 (s, 1H), 2.40-2.32 (m, 1H), 2.29 (s, 3H), 2.23-2.12 (m, 1H), 1.90-1.54 (m, 5H), 1.54-1.36 (m, 6H), 1.34-1.19 (m, 3H), 1.10 (t, J=7.0 Hz, 2H).

### Example 5: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(prop-1-en-2-yl)phenyl]pyrrolidin-1-yl]-7-azaspiro[3.5]nonan-7-yl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N₁,N₂-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), DCM (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-[2-(prop-1-en-2-yl)phenyl]pyrrolidine-1-carboxylate: To a stirred solution of tert-butyl (2S)-2-(2-bromophenyl)pyrrolidine-1-carboxylate (1.0 g, 3.1 mmol, 1.0 eq) and 4,4,5,5-tetramethyl-2-(prop-1-en-2-yl)-1,3,2-dioxaborolane (1.0 g, 6.1 mmol, 2.0 eq) in 1,4-dioxane (9 mL) and water (1 mL) were added K₂CO₃ (1.3 g, 9.3 mmol, 3.0 eq) and Pd(dqpf)Cl₂·CH₂Cl₂ (252 mg, 0.3 mmol, 0.1 eq). The resulting mixture was stirred for overnight at 80° C. under nitrogen atmosphere. The mixture was allowed to cool down to 25° C. The resulting mixture was concentrated under vacuum. The crude residue was purified by a flash column (silica gel, ethyl acetate/petroleum ether=1: 10). The crude product was purified by reverse phase flash with the following conditions: water (0.05% TFA) and CH₃CN to afford tert-butyl (2S)-2-[2-(prop-1-en-2-yl)phenyl]pyrrolidine-1-carboxylate (530 mg, 60.2%) as a light yellow solid. ¹HNMR (300 MHz, DMSO-d₆) δ 7.21 (dt, J=21.3, 7.4 Hz, 2H), 7.12-7.05 (m, 2H), 5.26 (s, 1H), 4.87 (d, J=7.0 Hz, 1H), 4.81 (s, 1H), 3.59 (ddd, J=10.5, 7.4, 5.4 Hz, 1H), 3.47 (dt, J=10.6, 7.2 Hz, 1H), 2.29-2.24 (m, 1H), 2.06 (S, 3H), 1.82 (dtp, J=17.9, 12.4, 6.6, 6.2 Hz, 2H), 1.65 (s, 1H), 1.37 (s, 3H), 1.08 (s, 6H).

Synthesis of (2S)-2-[2-(prop-1-en-2-yl)phenyl]pyrrolidine: To a stirred solution of tert-butyl (2S)-2-[2-(prop-1-en-2-yl)phenyl]pyrrolidine-1-carboxylate (480 mg, 1.7 mmol, 1.0 eq) in CH₂Cl₂ (8 mL) were added TFA (1 mL). The resulting mixture was stirred for overnight at 25° C. The resulting mixture was diluted with water (50 mL). The mixture was basified to pH 8 with saturated Na₂CO₃ (aq.). The resulting mixture was extracted with CH₂Cl₂ (3×50 mL). The combined organic layers were washed with brine (1×200 mL), dried over anhydrous Na₂SO₄. After filtration, the filtrate was concentrated under reduced pressure to afford (2S)-2-[2-(prop-1-en-2-yl)phenyl]pyrrolidine (300 mg, 95.9%) as a yellow solid. ¹HNMR (300 MHz, CDCl₃-d) δ 7.55 (dd, J=7.6, 1.7 Hz, 1H), 7.28-7.15 (m, 2H), 7.12 (dd, J=7.3, 1.7 Hz, 1H), 6.78 (s, 1H), 5.22 (p, J=1.6 Hz, 1H), 4.81 (dd, J=2.2, 1.1 Hz, 1H), 4.51 (dd, J=9.4, 7.0 Hz, 1H), 3.26 (dt, J=10.8, 7.3 Hz, 1H), 3.05 (ddd, J=10.9, 8.2, 6.0 Hz, 1H), 2.27-2.15 (m, 1H), 2.09 (d, J=4.7 Hz, 1H), 2.05 (t, J=1.3 Hz, 3H), 1.99-1.78 (m, 2H).

Synthesis of N-{3-nitro-4-[(oxan-4-ylmethyl)amino]benzenesulfonyl}-4-(2-{1-[2-(prop-1-en-2-yl)phenyl]pyrrolidin-2-yl}-7-azaspiro[3.5]nonan-7-yl)-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1.0 eq) and 1-[2-(prop-1-en-2-yl)phenyl]pyrrolidine (13 mg, 0.1 mmol, 1.0 eq) in CH₂Cl₂ (3 mL) were added NaBH(OAc)₃ (28 mg, 0.1 mmol, 2.0 eq). The resulting mixture was stirred for overnight at 25° C. The resulting mixture was concentrated under vacuum. The residue was purified by Prep-TLC (dichloromethane/methanol=12: 1) to afford N-{3-nitro-4-[(oxan-4-ylmethyl)amino]benzenesulfonyl}-4-(2-{1-[2-(prop-1-en-2-yl)phenyl]pyrrolidin-2-yl}-7-azaspiro[3.5]nonan-7-yl)-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (50 mg, 70.0%) as a yellow solid. LC-MS (ESI, m/z) M+1: 1073.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(prop-1-en-2-yl)phenyl]pyrrolidin-1-yl]-7-azaspiro[3.5]nonan-7-yl}benzamide hydrochloride: To a stirred solution of N-{3-nitro-4-[(oxan-4-ylmethyl)amino]benzenesulfonyl}-4-{2-[(2S)-2-[2-(prop-1-en-2-yl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (50 mg, 0.1 mmol, 1.0 eq) in TBAF/THF (1.0 M, 5 mL) were added ethylenediamine (60 mg, 0.1 mmol, 20.0 eq). The resulting mixture was stirred for 3 h at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(prop-1-en-2-yl)phenyl]pyrrolidin-1-yl]-7-azaspiro[3.5]nonan-7-yl}benzamide hydrochloride (14 mg, 30.7%) as a yellow solid. LC-MS (ESI, m/z) M+1: 943. ¹HNMR (300 MHz, DMSO-d₆) δ 12.30 (s, 1H), 11.23 (s, 2H), 8.73-8.12 (m, 3H), 7.62 (d, J=8.9 Hz, 1H), 7.50-7.29 (m, 3H), 7.28-7.01 (m, 2H), 6.96-6.68 (m, 3H), 6.58 (s, 1H), 6.01 (d, J=12.4 Hz, 1H), 5.31 (s, 1H), 4.81 (s, 2H), 4.38 (dt, J=29.6, 8.6 Hz, 5H), 3.95-3.49 (m, 6H), 3.46-2.99 (m, 9H), 2.46-2.35 (m, 1H), 2.26 (d, J=10.2 Hz, 1H), 2.06 (d, J=19.0 Hz, 6H), 1.89 (s, 1H), 1.63 (d, J=12.5 Hz, 2H), 1.56-1.13 (m, 9H).

### Example 6: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-ethenylphenyl)pyrrolidin-1-yl]-7-azaspiro[3.5]nonan-7-yl}-N-{3-nitro-4-[(oxan-4-ylmethyl)amino]benzenesulfonyl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), DCM (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-(2-ethenylphenyl)pyrrolidine-1-carboxylate: To a stirred solution of tert-butyl (2S)-2-(2-bromophenyl)pyrrolidine-1-carboxylate (700 mg, 2.1 mmol, 1.0 eq) and 2-ethenyl-4,4,5,5-tetramethyl-1,3,2-dioxaborolane (663 mg, 4.3 mmol, 2.0 eq) in 1,4-dioxane (9 mL) and water (1 mL) were added K₂CO₃ (892 mg, 6.4 mmol, 3.0 eq) and Pd(dqpf)Cl₂·CH₂Cl₂ (176 mg, 0.2 mmol, 0.1 eq). The resulting mixture was stirred for overnight at 80° C. under nitrogen atmosphere. The mixture was allowed to cool down to 25° C. The resulting mixture was concentrated under vacuum. The crude residue was purified by a flash column (silica gel, ethyl acetate/petroleum ether=1: 10) to afford to afford tert-butyl (2S)-2-(2-ethenylphenyl)pyrrolidine-1-carboxylate (559 mg, 95.3%) as a yellow oil. LC-MS (ESI, m/z) M-t-Bu+CH₃CN: 259.

Synthesis of (2S)-2-(2-ethenylphenyl)pyrrolidine: To a stirred solution of tert-butyl (2S)-2-(2-ethenylphenyl)pyrrolidine-1-carboxylate (550 mg, 2.0 mmol, 1.0 eq) in CH₂Cl₂ (18 mL) were added TFA (2 mL). The resulting mixture was stirred for overnight at 25° C. The mixture was basified to pH 8 with saturated Na₂CO₃ (aq.). The resulting mixture was extracted with CH₂Cl₂ (3×50 mL). The combined organic layers were washed with brine (1×200 mL), dried over anhydrous Na₂SO₄. After filtration, the filtrate was concentrated under reduced pressure to afford (2S)-2-(2-ethenylphenyl)pyrrolidine (193 mg, 55.4%) as a yellow oil. ¹HNMR (300 MHz, DMSO-de) δ 7.58 (dd, J=7.6, 1.6 Hz, 1H), 7.46 (dd, J=7.5, 1.6 Hz, 1H), 7.24 (td, J=7.5, 1.6 Hz, 1H), 7.23-7.15 (m, 1H), 7.13 (dd, J=17.4, 11.0 Hz, 1H), 5.65 (dd, J=17.4, 1.6 Hz, 1H), 5.30 (dd, J=11.0, 1.7 Hz, 1H), 4.32 (t, J=7.7 Hz, 1H), 3.03 (ddd, J=9.7, 7.0, 5.6 Hz, 1H), 2.91 (dt, J=9.7, 7.4 Hz, 1H), 2.18-2.05 (m, 1H), 1.80-1.68 (m, 2H), 1.36 (dq, J=12.0, 7.9 Hz, 1H).

Synthesis of 4-{2-[(2S)-2-(2-ethenylphenyl)pyrrolidin-1-yl]-7-azaspiro[3.5]nonan-7-yl}-N-{3-nitro. 4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1.0 eq) and (2S)-2-(2-ethenylphenyl)pyrrolidine (12 mg, 0.1 mmol, 1.0 eq) in CH₂Cl₂ (3 mL) were added NaBH(OAc)₃ (28 mg, 0.1 mmol, 2.0 eq). The resulting mixture was stirred for overnight at 25° C. The resulting mixture was concentrated under vacuum. The residue was purified by Prep-TLC (dichloromethane/methanol=10:1) to afford 4-{2-[(2S)-2-(2-ethenylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (56 mg, 79.4%) as a yellow solid. LC-MS (ESI, m/z) M+1: 1059.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-ethenylphenyl)pyrrolidin-1-yl]-7-azaspiro[3.5]nonan-7-yl}-N-{3-nitro-4-[(oxan-4-ylmethyl)amino]benzenesulfonyl}benzamide hydrochloride: To a stirred solution of 4-{2-[(2S)-2-(2-ethenylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (56 mg, 0.1 mmol, 1.0 eq) in TBAF/THF (1.0 M, 5 mL) were added ethylenediamine (64 mg, 1.1 mmol, 20.0 eq). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 7 min); Detector, uv. The collected solution was concentrated under vacuum to remove CH₃CN and the resulting solution was dried by lyophilization (added with Conc.HCl (1 drop)). This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-ethenylphenyl)pyrrolidin-1-yl]-7-azaspiro[3.5]nonan-7-yl}-N-{3-nitro-4-[(oxan-4-ylmethyl)amino]benzenesulfonyl}benzamide hydrochloride (15 mg, 30.5%) as a yellow solid. LC-MS (ESI, m/z) M+1: 929. ¹HNMR (300 MHz, DMSO-d₆) δ 12.17 (d, J=79.5 Hz, 1H), 11.12 (d, J=64.8 Hz, 2H), 8.50 (d, J=30.6 Hz, 1H), 8.31 (d, J=2.2 Hz, 1H), 8.09 (s, 1H), 7.62 (d, J=8.9 Hz, 1H), 7.56-7.46 (m, 1H), 7.42 (d, J=9.7 Hz, 3H), 7.28-7.01 (m, 3H), 6.95-6.70 (m, 3H), 6.58 (s, 1H), 6.01 (d, J=10.9 Hz, 1H), 5.71 (d, J=17.1 Hz, 1H), 5.42 (d, J=11.1 Hz, 1H), 4.74 (s, 1H), 4.58 (t, J=8.3 Hz, 1H), 4.32 (t, J=7.8 Hz, 2H), 3.87 (d, J=15.1 Hz, 5H), 3.69 (s, 1H), 3.54 (d, J=9.2 Hz, 1H), 3.41-2.97 (m, 10H), 2.27 (s, 1H), 2.10 (s, 4H), 1.63 (d, J=12.8 Hz, 2H), 1.36 (d, J=45.5 Hz, 9H).

### Example 7: Preparation of 4-{2-[(2S)-2-(2-ethylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), DCM (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-(2-ethylphenyl)pyrrolidine-1-carboxylate: To a stirred solution of tert-butyl (2S)-2-(2-bromophenyl)pyrrolidine-1-carboxylate (2.5 g, 7.7 mmol, 1.0 eq) and boronic acid, ethyl-(1.7 g, 23.1 mmol, 3.0 eq) in dioxane (27 mL) and water (3 mL) were added K₂CO₃ (3.2 g, 23.0 mmol, 3.0 eq) and Pd(dqpf)Cl₂·CH₂Cl₂ (628 mg, 0.8 mmol, 0.1 eq). The resulting mixture was stirred for overnight at 80° C. under nitrogen atmosphere. The mixture was allowed to cool down to 25° C. The resulting mixture was concentrated under vacuum. The crude residue was purified by a flash column (silica gel, ethyl acetate/petroleum ether=1: 10) to afford tert-butyl (2S)-2-(2-ethylphenyl)pyrrolidine-1-carboxylate (2 g, 85.3%) as a colorless oil. ¹HNMR (300 MHz, CDCl₃-d) δ 7.21-6.98 (m, 4H), 5.26-4.95 (m, 1H), 3.67 (ddt, J=18.7, 11.4, 6.0 Hz, 2H), 2.70 (dd, J=9.5, 5.6 Hz, 1H), 2.47-2.22 (m, 1H), 2.00-1.72 (m, 3H), 1.48 (s, 3H), 1.19 (d, J=8.0 Hz, 6H).

Synthesis of (2S)-2-(2-ethylphenyl)pyrrolidine: To a stirred solution of tert-butyl (2S)-2-(2-ethylphenyl)pyrrolidine-1-carboxylate (632 mg, 2.3 mmol, 1.0 eq) in CH₂Cl₂ (8 mL) were added TFA (1 mL). The resulting mixture was stirred for overnight at 25° C. The resulting mixture was diluted with water (50 mL). The mixture was basified to pH 8 with saturated Na₂CO₃ (aq.). The resulting mixture was extracted with CH₂Cl₂ (3×50 mL). The combined organic layers were washed with brine (1×200 mL), dried over anhydrous Na₂SO₄. After filtration, the filtrate was concentrated under reduced pressure to afford (2S)-2-(2-ethylphenyl)pyrrolidine (313 mg, 77.8%) as a yellow oil. LC-MS (ESI, m/z) M+1: 176.

Synthesis of 4-{2-[(2S)-2-(2-ethylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1.0 eq) and (2S)-2-(2-ethylphenyl)pyrrolidine (12 mg, 0.1 mmol, 1.0 eq) in CH₂Cl₂ (3 mL) were added NaBH(OAc)₃ (28 mg, 0.1 mmol, 2.0 eq). The resulting mixture was stirred for overnight at 25° C. The resulting mixture was concentrated under vacuum. The residue was purified by Prep-TLC (dichloromethane/methanol=12: 1) to afford 4-{2-[(2S)-2-(2-ethylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (53 mg, 75.1%) as a yellow solid. LC-MS (ESI, m/z) M+1: 1061.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-ethylphenyl)pyrrolidin-1-yl]-7-azaspiro[3.5]nonan-7-yl}-N-{3-nitro. 4-[(oxan-4-ylmethyl)amino]benzenesulfonyl}benzamide hydrochloride: To a stirred solution of 4-{2-[(2S)-2-(2-ethylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (53 mg, 0.1 mmol, 1.0 eq) in TBAF/THF (1.0 M, 5 mL) were added ethylenediamine (60 mg, 0.1 mmol, 20.0 eq). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-ethylphenyl)pyrrolidin-1-yl]-7-azaspiro[3.5]nonan-7-yl}-N-{3-nitro-4-[(oxan-4-ylmethyl)amino]benzenesulfonyl}benzamide hydrochloride (16 mg, 33.1%) as a yellow solid. LC-MS (ESI, m/z) M+1: 931. ¹HNMR (300 MHz, DMSO-d₆) δ 12.31 (s, 1H), 11.13 (d, J=63.7 Hz, 2H), 8.70-8.06 (m, 3H), 7.65 (dd, J=20.1, 8.2 Hz, 1H), 7.50-7.38 (m, 1H), 7.35-7.00 (m, 5H), 6.95-6.69 (m, 3H), 6.58 (s, 1H), 6.05-5.96 (m, 1H), 4.46 (d, J=9.6 Hz, 2H), 4.31 (q, J=9.8, 8.7 Hz, 2H), 4.02-3.75 (m, 4H), 3.70 (dd, J=11.7, 6.0 Hz, 1H), 3.59-3.46 (m, 1H), 3.39-2.98 (m, 9H), 2.78 (dt, J=14.2, 7.0 Hz, 1H), 2.63 (dd, J=14.3, 7.1 Hz, 1H), 2.45-2.36 (m, 1H), 2.34-2.24 (m, 1H), 2.19-1.80 (m, 5H), 1.63 (d, J=12.8 Hz, 2H), 1.55-1.20 (m, 8H), 1.13 (t, J=7.4 Hz, 3H).

### Example 8: Preparation of 4-{2-[(2S)-2-(2-cyclopropylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), DCM (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-(2-cyclopropylphenyl)pyrrolidine-1-carboxylate: To a stirred solution of tert-butyl (2S)-2-(2-bromophenyl)pyrrolidine-1-carboxylate (1.5 g, 4.6 mmol, 1.0 eq) and cyclopropylboronic acid (1.2 g, 13.9 mmol, 3.0 eq) in 1,4-dioxane (18 mL) and water (2 mL) were added K₂CO₃ (1.9 g, 13.9 mmol, 3.0 eq) and Pd(PPh₃)₄(534 mg, 0.5 mmol, 0.1 eq). The resulting mixture was stirred 6 hours at 90° C. under nitrogen atmosphere. The mixture was allowed to cool down to 25° C. The resulting mixture was concentrated under vacuum. The crude residue was purified by a flash column (silica gel, ethyl acetate/petroleum ether=1: 15) to afford tert-butyl (2S)-2-(2-cyclopropylphenyl)pyrrolidine-1-carboxylate (1 g, 91.6%) as a light yellow oil. ¹HNMR (300 MHz, CDCl₃-d) δ 7.22-6.92 (m, 4H), 5.59-5.33 (m, 1H), 3.82-3.45 (m, 2H), 2.40 (dt, J=17.2, 8.4 Hz, 1H), 2.03-1.76 (m, 4H), 1.56-1.39 (m, 3H), 1.22-1.10 (m, 6H), 1.05-0.84 (m, 3H), 0.70 (ddt, J=29.3, 9.3, 4.2 Hz, 2H).

Synthesis of (2S)-2-(2-cyclopropylphenyl)pyrrolidine: A solution of tert-butyl (2S)-2-(2-cyclopropylphenyl)pyrrolidine-1-carboxylate (1.0 g, 4.2 mmol, 1.0 eq) and in CH₂Cl₂ (18 mL) was added TFA (2 mL). The resulting mixture was stirred overnight at 25° C. The resulting mixture was concentrated under vacuum. The mixture was basified to pH 8 with saturated NaHCO₃ (aq.). The resulting mixture was extracted with CH₂Cl₂ (3×100 mL). The combined organic layers were washed with brine (1×300 mL), dried over anhydrous Na₂SO₄. After filtration, the filtrate was concentrated under reduced pressure. This resulted in (2S)-2-(2-cyclopropylphenyl)pyrrolidine (760 mg, 97.2%) as a yellow oil. ¹HNMR (300 MHz, CDCl₃-d) δ 7.52-7.44 (m, 1H), 7.16 (ddd, J=12.9, 6.8, 3.2 Hz, 2H), 7.02 (dd, J=6.6, 2.4 Hz, 1H), 5.42 (s, 2H), 4.76 (t, J=7.7 Hz, 1H), 3.26 (ddd, J=10.2, 7.5, 5.6 Hz, 1H), 3.07 (dt, J=10.2, 7.5 Hz, 1H), 2.28 (dtd, J=12.5, 7.7, 5.0 Hz, 1H), 2.10-1.79 (m, 2H), 1.70 (ddd, J=16.2, 10.4, 7.7 Hz, 1H), 0.94 (dd, J=8.5, 2.1 Hz, 2H), 0.78-0.60 (m, 2H).

Synthesis of 4-{2-[(2S)-2-(2-cyclopropylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of (2S)-2-(2-cyclopropylphenyl)pyrrolidine (12 mg, 0.1 mmol, 1.0 eq) and N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1.0 eq) in CH₂Cl₂ (3 mL) were added NaBH(OAc)₃ (28 mg, 0.1 mmol, 2.0 eq). The resulting mixture was stirred overnight at 25° C. The resulting mixture was concentrated under vacuum. The residue was purified by Prep-TLC (Ethyl acetate) to afford 4-{2-[(2S)-2-(2-cyclopropylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (70 mg, 98.0%) as a yellow solid. LC-MS (ESI, m/z) M+1: 1073.

Synthesis of 4-{2-[(2S)-2-(2-cyclopropylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]. N-{3-nitro-4-[(oxan-4-ylmethyl)amino]benzenesulfonyl}benzamide hydrochloride: To a stirred solution of 4-{2-[(2S)-2-(2-cyclopropylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (70 mg, 0.1 mmol, 1.0 eq) in TBAF/THF (1.0 M, 5 mL) was added ethylenediamine (78 mg, 1.3 mmol, 20.0 eq). The resulting mixture was stirred for 3 hours at 70° C. The mixture was allowed to cool down to 25° C. The resulting mixture was diluted with water (50 mL). The resulting mixture was extracted with Ethyl acetate (3×50 mL). The combined organic layers were washed with brine (1×200 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 4-{2-[(2S)-2-(2-cyclopropylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide hydrochloride (20 mg, 31.3%) as a yellow solid. LC-MS (ESI, m/z) M+1: 943. ¹HNMR (300 MHz, DMSO-d₆) δ 12.32 (s, 1H), 11.58-10.98 (m, 2H), 8.78-8.01 (m, 3H), 7.77-6.51 (m, 10H), 6.05-5.96 (m, 1H), 4.97 (d, J=8.6 Hz, 1H), 4.61-4.52 (m, 1H), 4.42-4.00 (m, 5H), 4.03-3.47 (m, 5H), 3.39-2.98 (m, 8H), 2.45-2.23 (m, 3H), 2.23-1.81 (m, 6H), 1.73-1.13 (m, 9H), 0.95 (q, J=10.0, 9.1 Hz, 2H), 0.64 (d, J=6.0 Hz, 2H).

### Example 9: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{6-[(2S)-2-(2-isopropylphenyl)pyrrolidin-1-yl]-2-azaspiro[3.3]heptan-2-yl}-N-{3-nitro-4-[(oxan-4-ylmethyl)amino]benzenesulfonyl}benzamide

Synthesis of tert-butyl 6-[(2S)-2-(2-bromophenyl)pyrrolidin-1-yl]-2-azaspiro[3.3]heptane-2-carboxylate: Into a 250 mL round-bottom flask, were placed tert-butyl 6-oxo-2-azaspiro[3.3]heptane-2-carboxylate (9.8 g, 46.4 mmol, 1.1 eq), (2S)-2-(2-bromophenyl)pyrrolidine (10.0 g, 44.2 mmol, 1.0 eq), NaBH(OAc)₃ (23.4 g, 110.5 mmol, 2.5 eq). This was followed by the addition of CH₂Cl₂ (100 mL), in portions at 25° C. The resulting solution was stirred for 2 hours at 25° C. The reaction was then quenched by the addition of 200 mL of water. The resulting solution was extracted with 100 mL of dichloromethane. The resulting mixture was washed with 3×100 mL of water and 3×100 mL of brine. The mixture was dried over anhydrous Na₂SO₄ and concentrated. The crude residue was applied onto a silica gel column with ethyl acetate/petroleum ether (1: 1). This resulted in (12 g, 64.3%) of tert-butyl 6-[(2S)-2-(2-bromophenyl)pyrrolidin-1-yl]-2-azaspiro[3.3]heptane-2-carboxylate as white solid. LC-MS (ESI, m/z) M+1: 421/423.

Synthesis of tert-butyl 6-[(2S)-2-[2-(prop-1-en-2-yl)phenyl]pyrrolidin-1-yl]-2-azaspiro[3.3]heptane-2-carboxylate: Into a 250 mL round-bottom flask purged and maintained with an inert atmosphere of nitrogen, were placed 4,4,5,5-tetramethyl-2-(prop-1-en-2-yl)-1,3,2-dioxaborolane (4.0 g, 23.7 mmol, 2.0 eq), tert-butyl 6-[(2S)-2-(2-bromophenyl)pyrrolidin-1-yl]-2-azaspiro[3.3]heptane-2-carboxylate (5.0 g, 11.8 mmol, 1.0 eq), K₂CO₃ (4.9 g, 35.6 mmol, 3.0 eq), Pd(dqpf)Cl₂ (0.9 g, 1.2 mmol, 0.1 eq), Dioxane (50 mL), water (10 mL). The resulting solution was stirred for 8 hours at 80° C. The resulting mixture was concentrated. The crude residue was purified by a flash column (silica gel, ethyl acetate/petroleum ether=1:1). This resulted in (2.3 g, 50.6%) of tert-butyl 6-[(2S)-2-[2-(prop-1-en-2-yl)phenyl]pyrrolidin-1-yl]-2-azaspiro[3.3]heptane-2-carboxylate as white solid. LC-MS (ESI, m/z) M+1: 383.

Synthesis of tert-butyl 6-[(2S)-2-(2-isopropylphenyl)pyrrolidin-1-yl]-2-azaspiro[3.3]heptane-2-carboxylate: Into a 100 mL round-bottom flask, were placed tert-butyl 6-[(2S)-2-[2-(prop-1-en-2-yl)phenyl]pyrrolidin-1-yl]-2-azaspiro[3.3]heptane-2-carboxylate (2.2 g, 5.7 mmol, 1.0 eq), EtOH (30 mL), Pd/C (0.3 g, 2.8 mmol, 0.5 eq), H₂ (3 atm). The resulting solution was stirred for 16 hours at 25° C. The solids were filtered out. The resulting mixture was concentrated. This resulted in (1.5 g, 67.8%) of tert-butyl 6-[(2S)-2-(2-isopropylphenyl)pyrrolidin-1-yl]-2-azaspiro[3.3]heptane-2-carboxylate as colorless oil. LC-MS (ESI, m/z) M+1: 385.

Synthesis of (S)-6-(2-(2-isopropylphenyl)pyrrolidin-1-yl)-2-azaspiro[3.3]heptane: Into a 50 mL round-bottom flask, were placed tert-butyl 6-[(2S)-2-(2-isopropylphenyl)pyrrolidin-1-yl]-2-azaspiro[3.3]heptane-2-carboxylate (1.5 g, 3.9 mmol, 1.0 eq), CH₂Cl₂ (15 mL), TFA (3.0 mL). The resulting solution was stirred for 6 hours at 25° C. The pH value of the solution was adjusted to 8 with NaHCO₃ (1 M). The resulting solution was extracted with 3×20 mL of dichloromethane. The resulting mixture was washed with 3×20 mL of brine. The solid was dried in an oven under reduced pressure. This resulted in (1 g, 85.3%) of (S)-6-(2-(2-isopropylphenyl)pyrrolidin-1-yl)-2-azaspiro[3.3]heptane as colorless oil. LC-MS (ESI, m/z) M+1: 285.

Synthesis of methyl (S)-2-bromo-4-(6-(2-(2-isopropylphenyl)pyrrolidin-1-yl)-2-azaspiro[3.3]heptan-2-yl)benzoate: Into a 8 mL sealed tube, were placed methyl 2-bromo-4-fluorobenzoate (247 mg, 1.1 mmol, 1.0 eq), 6-[(2S)-2-[2-(prop-1-en-2-yl)phenyl]pyrrolidin-1-yl]-2-azaspiro[3.3]heptane (300 mg, 1.1 mmol, 1.0 eq), DMSO (5.0 mL), Na₂CO₃ (337 mg, 3.2 mmol, 3.0 eq). The resulting solution was stirred for 8 hours at 110° C. The reaction was then quenched by the addition of 10 mL of water/ice. The solids were collected by filtration. The crude residue was purified by a flash column (silica gel, ethyl acetate/petroleum ether=1:1). This resulted in (270 mg, 51.3%) of methyl 4-(6-((S)-2-(2-isopropylphenyl)pyrrolidin-1-yl)-2-azaspiro[3.3]heptan-2-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzoate as colorless oil. LC-MS (ESI, m/z) M+1: 497

Synthesis of methyl 4-(6-((S)-2-(2-isopropylphenyl)pyrrolidin-1-yl)-2-azaspiro[3.3]heptan-2-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzoate: Into a 8 mL sealed tube were added (11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (100 mg, 0.3 mmol, 1.0 eq) and methyl 2-bromo-4-[6-[(2S)-2-(2-isopropylphenyl)pyrrolidin-1-yl]-2-azaspiro[3.3]heptan-2-yl]benzoate (286 mg, 0.6 mmol, 2.0 eq) and Pd2(dba)3 (13 mg, 0.01 mmol, 0.05 eq) and xantphos (83 mg, 0.1 mmol, 0.5 eq) and Cs₂CO₃ (281 mg, 0.9 mmol, 3.0 eq) and toluene (5 mL). The resulting mixture was stirred for additional 5 hours at 100° C. The resulting mixture was concentrated under reduced pressure. The crude product was purified by a flash column (silica gel, ethyl acetate/petroleum ether=1:1) to afford methyl 4-[6-[(2S)-2-(2-isopropylphenyl)pyrrolidin-1-yl]-2-azaspiro[3.3]heptan-2-yl]-2-[(11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraen-10-yl]benzoate (100 mg, 45.4%) as white solid. LC-MS (ESI, m/z) M+1:764.

Synthesis of 4-(6-((S)-2-(2-isopropylphenyl)pyrrolidin-1-yl)-2-azaspiro[3.3]heptan-2-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzoic acid: Into a 8 mL sealed tube were added methyl 4-{6-[2-(2-isopropylphenyl)pyrrolidin-1-yl]-2-azaspiro[3.3]heptan-2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (100 mg, 0.1 mmol, 1.0 eq), NaOH (20 mg, 0.5 mmol, 4 eq), MeOH (1.0 mL), dioxane (1.0 mL) and water (0.5 mL) The resulting mixture was stirred for 6 hours at 70° C. The mixture was acidified to pH=6 with 1 M HCl. The aqueous layer was extracted with CH₂Cl₂ (2×2 mL). The resulting powder was dried under vacuum. The resulting mixture was concentrated under reduced pressure. This resulted in (50 mg, 51.2%) of 4-(6-((S)-2-(2-isopropylphenyl)pyrrolidin-1-yl)-2-azaspiro[3.3]heptan-2-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzoic acid as yellow solid. LC-MS (ESI, m/z) M+1:750.

Synthesis of 4-(6-((S)-2-(2-isopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin. 5(1H)-yl)benzamide: Into a 8 mL sealed tube were added 4-{6-[(2S)-2-(2-isopropylphenyl)pyrrolidin-1-yl]-2-azaspiro[3.3]heptan-2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoic acid (50 mg, 0.07 mmol, 1.0 eq) and 3-nitro-4-[(oxan-4-ylmethyl)amino]benzenesulfonamide (21 mg, 0.07 mmol, 1.0 eq) and CH₂Cl₂ (2 mL) and DMAP (12 mg, 0.1 mmol, 1.5 eq) and EDCI (15 mg, 0.08 mmol, 1.2 eq). The resulting mixture was stirred for overnight at 25° C. The resulting mixture was concentrated under reduced pressure. The crude was purified by a flash column (silica gel, petroleum ether/ethyl acetate=1:1) to afford 4-(6-((S)-2-(2-isopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (50 mg, 71.6%) as yellow solid. LC-MS (ESI, m/z) M+1:1047.

Synthesis of 2-[(11R,15S)-13,16-dioxa-2,4,10-triazatetracyclo[7.7.0.0\hat{ }{3,7}.0\hat{ }{11,15}]hexadeca-1 (9),2,5,7-tetraen-10-yl]-4-{6-[(2S)-2-(2-isopropylphenyl)pyrrolidin-1-yl]-2-azaspiro[3.3]heptan-2-yl}-N-{3-nitro-4-[(oxan-4-ylmethyl)amino]benzenesulfonyl}benzamide: Into a 2 mL vial were added 4-{6-[(2S)-2-(2-isopropylphenyl)pyrrolidin-1-yl]-2-azaspiro[3.3]heptan-2-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (28 mg, 0.03 mmol, 1.0 eq), TBAF (0.5 mL, 1.9 mmol, 71.5 eq) and ethylenediamine (24 mg, 0.4 mmol, 15 eq) at 25° C. The resulting mixture was stirred for 16 hours at 70° C. The mixture was allowed to cool down to 25° C. The resulting mixture was diluted with water (5 mL). The resulting mixture was extracted with Ethyl acetate (3×5 mL). The combined organic layers were washed with brine (1×5 mL), dried over anhydrous Na₂SO₄. After filtration, the filtrate was concentrated under reduced pressure. The crude residue was purified by reverse flash choursomatography with the following conditions: Column, xBridge Shield RP18 OBD Column, 19*150 mm, 5 μm; mobile phase, Water (10 mmol/L NH₄HCO₃ and 0.1% NH₃·H₂O) and CH₃CN (37% CH₃CN up to 55% in 7 min); UV 254 nm. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{6-[(2S)-2-(2-isopropylphenyl)pyrrolidin-1-yl]-2-azaspiro[3.3]heptan-2-yl}-N-{3-nitro-4-[(oxan-4-ylmethyl)amino]benzenesulfonyl}benzamide (10 mg, 40.7%) as light yellow solid. LC-MS (ESI, m/z) M+1: 917. ¹HNMR:(300 MHz, DMSO-d₆) δ 12.27 (s, 1H), 11.19-11.01 (m, 1H), 8.33 (d, J=27.1 Hz, 1H), 7.58 (d, J=8.1 Hz, 2H), 7.39 (s, 1H), 7.24 (s, 1H), 7.15 (s, 1H), 7.09 (d, J=12.4 Hz, 1H), 7.01 (d, J=7.7 Hz, 1H), 6.79 (s, 1H), 6.56 (s, 1H), 6.28 (s, 1H), 6.20 (d, J=7.8 Hz, 1H), 6.07 (d, J=10.4 Hz, 1H), 5.99 (s, 1H), 4.64 (s, 1H), 4.43 (d, J=8.6 Hz, 1H), 4.28 (s, 1H), 4.08 (d, J=7.8 Hz, 1H), 3.87 (d, J=11.6 Hz, 3H), 3.80 (s, 1H), 3.73-3.63 (m, 4H), 3.54 (t, J=9.8 Hz, 1H), 3.47 (s, 2H), 3.22 (s, 1H), 3.08 (s, 1H), 2.82 (s, 2H), 2.26 (s, 1H), 2.08 (s, 4H), 2.00 (d, J=5.4 Hz, 3H), 1.78 (s, 1H), 1.71 (d, J=9.0 Hz, 1H), 1.63 (d, J=12.2 Hz, 2H), 1.54 (s, 1H), 1.36-1.06 (m, J=15.3 Hz, 8H).

### Example 10: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{6-[(2S)-2-[2-(prop-1-en-2-yl)phenyl]pyrrolidin-1-yl]-2-azaspiro[3.3]heptan-2-yl}benzamide

Synthesis of tert-butyl 6-[(2S)-2-(2-bromophenyl)pyrrolidin-1-yl]-2-azaspiro[3.3]heptane-2-carboxylate: Into a 250 mL round-bottom flask, were placed tert-butyl 6-oxo-2-azaspiro[3.3]heptane-2-carboxylate (9.8 g, 46.4 mmol, 1.1 eq), (2S)-2-(2-bromophenyl)pyrrolidine (10.0 g, 44.2 mmol, 1.0 eq), NaBH(OAc)₃ (23.4 g, 110.5 mmol, 2.5 eq). This was followed by the addition of CH₂Cl₂ (100 mL), in portions at 25° C. The resulting solution was stirred for 2 hours at 25° C. The reaction was then quenched by the addition of 200 mL of water. The resulting solution was extracted with 100 mL of dichloromethane. The resulting mixture was washed with 3×100 mL of water and 3×100 mL of brine. The mixture was dried over anhydrous Na₂SO₄ and concentrated. The crude residue was applied onto a silica gel column with ethyl acetate/petroleum ether (1:1). This resulted in (12 g, 64.3%) of tert-butyl 6-[(2S)-2-(2-bromophenyl)pyrrolidin-1-yl]-2-azaspiro[3.3]heptane-2-carboxylate as white solid. LC-MS (ESI, m/z) M+1: 421/423.

Synthesis of tert-butyl 6-[(2S)-2-[2-(prop-1-en-2-yl)phenyl]pyrrolidin-1-yl]-2-azaspiro[3.3]heptane-2-carboxylate: Into a 250 mL round-bottom flask purged and maintained with an inert atmosphere of nitrogen, were placed 4,4,5,5-tetramethyl-2-(prop-1-en-2-yl)-1,3,2-dioxaborolane (4.0 g, 23.7 mmol, 2.0 eq), tert-butyl 6-[(2S)-2-(2-bromophenyl)pyrrolidin-1-yl]-2-azaspiro[3.3]heptane-2-carboxylate (5.0 g, 11.8 mmol, 1.0 eq), K₂CO₃ (4.9 g, 35.6 mmol, 3.0 eq), Pd(dqpf)Cl₂ (0.9 g, 1.2 mmol, 0.1 eq), Dioxane (50 mL), water (10 mL). The resulting solution was stirred for 8 hours at 80° C. The resulting mixture was concentrated. The crude residue was purified by a flash column (silica gel, ethyl acetate/petroleum ether=1:1). This resulted in (2.3 g, 50.6%) of tert-butyl 6-[(2S)-2-[2-(prop-1-en-2-yl)phenyl]pyrrolidin-1-yl]-2-azaspiro[3.3]heptane-2-carboxylate as white solid. LC-MS (ESI, m/z) M+1: 383.

Synthesis of 2-bromo-4-{6-[(2S)-2-[2-(prop-1-en-2-yl)phenyl]pyrrolidin-1-yl]-2-azaspiro[3.3]heptan-2-yl}benzoate: Into a 8 mL sealed tube were added methyl 2-bromo-4-fluorobenzoate (247 mg, 1.1 mmol, 1.0 eq) and 6-[(2S)-2-[2-(prop-1-en-2-yl)phenyl]pyrrolidin-1-yl]-2-azaspiro[3.3]heptane (300 mg, 1.1 mmol, 1.0 eq) and Na₂CO₃ (337 mg, 3.1 mmol, 3 eq) in DMSO was stirred for 8 h at 110° C. under nitrogen atmosphere. The resulting mixture was diluted with water (mL). The resulting mixture was filtered, the filter was purified by a flash column (silica gel, ethyl acetate/petroleum ether=1:1) to afford methyl 2-bromo-4-{6-[(2S)-2-[2-(prop-1-en-2-yl)phenyl]pyrrolidin-1-yl]-2-azaspiro[3.3]heptan-2-yl}benzoate (270 mg, 51.3%) as colorless oil. LC-MS (ESI, m/z) M+1: 495/497.

Synthesis of methyl 4-{6-[(2S)-2-[2-(prop-1-en-2-yl)phenyl]pyrrolidin-1-yl]-2-azaspiro[3.3]heptan. 2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate: Into a 8 mL sealed tube were added (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (100 mg, 0.3 mmol, 1.0 eq), methyl 2-bromo-4-{6-[(2S)-2-[2-(prop-1-en-2-yl)phenyl]pyrrolidin-1-yl]-2-azaspiro[3.3]heptan-2-yl}benzoate (270 mg, 0.5 mmol, 2.0 eq), Pd₂(dba)₃·CHCl₃ (30 mg, 0.03 mmol, 0.1 eq), XantPhos (16 mg, 0.03 mmol, 0.1 eq) and Cs₂CO₃ (281 mg, 0.8 mmol, 3.0 eq) in Toluene (3 mL) was stirred for 5 hours at 100° C. under nitrogen atmosphere. The resulting mixture was concentrated under reduced pressure. The crude product was purified by a flash column (silica gel, ethyl acetate/petroleum ether=1:1) to afford methyl 4-{6-[(2S)-2-[2-(prop-1-en-2-yl)phenyl]pyrrolidin-1-yl]-2-azaspiro[3.3]heptan-2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (50 mg, 22.8%) as yellow oil. LC-MS (ESI, m/z) M+1: 762.

Synthesis of 4-{6-[(2S)-2-[2-(prop-1-en-2-yl)phenyl]pyrrolidin-1-yl]-2-azaspiro[3.3]heptan-2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoic acid: Into a 8 mL vial were added methyl 4-{6-[(2S)-2-[2-(prop-1-en-2-yl)phenyl]pyrrolidin-1-yl]-2-azaspiro[3.3]heptan-2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (46 mg, 0.06 mmol, 1.0 eq), MeOH (0.5 mL), water (0.5 mL), and NaOH (10 mg, 0.3 mmol, 4.0 eq) at 25° C. The resulting mixture was stirred for 3 hours at 50° C. The mixture was allowed to cool down to 25° C. The resulting mixture was diluted with water (The mixture was acidified to pH=6 with 1 M HCl). The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure. This resulted in 4-{6-[(2S)-2-[2-(prop-1-en-2-yl)phenyl]pyrrolidin-1-yl]-2-azaspiro[3.3]heptan-2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoic acid (30 mg, 66.4%) as light yellow solid. LC-MS (ESI, m/z) M+1: 748.

Synthesis of N-{3-nitro-4-[(oxan-4-ylmethyl)amino]benzenesulfonyl}-4-{6-[(2S)-2-[2-(prop-1-en-2-yl)phenyl]pyrrolidin-1-yl]-2-azaspiro[3.3]heptan-2-yl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 8 mL vial were added 4-{6-[(2S)-2-[2-(prop-1-en-2-yl)phenyl]pyrrolidin-1-yl]-2-azaspiro[3.3]heptan-2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoic acid (30 mg, 0.04 mmol, 1.0 eq), 3-nitro-4-[(oxan-4-ylmethyl)amino]benzenesulfonamide (12 mg, 0.04 mmol, 1 eq), DMAP (10 mg, 0.08 mmol, 2.0 eq), CH₂Cl₂ (1.0 mL) and EDCI (9.2 mg, 0.05 mmol, 1.2 eq) at 25° C. The resulting mixture was stirred for 3 hours at 25° C. The resulting mixture was diluted with water (10 mL). The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure. The crude residue was purified by a flash column (silica gel, dichloromethane/methanol=95: 5) to afford N-{3-nitro-4-[(oxan-4-ylmethyl)amino]benzenesulfonyl}-4-{6-[(2S)-2-[2-(prop-1-en-2-yl)phenyl]pyrrolidin-1-yl]-2-azaspiro[3.3]heptan-2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (31 mg, 73.9%) as light yellow solid. LC-MS (ESI, m/z) M+1: 1046.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{6-[(2S)-2-[2-(prop-1-en-2-yl)phenyl]pyrrolidin-1-yl]-2-azaspiro[3.3]heptan-2-yl}benzamide: Into a 2 mL vial were added N-{3-nitro-4-[(oxan-4-ylmethyl)amino]benzenesulfonyl}-4-{6-[(2S)-2-[2-(prop-1-en-2-yl)phenyl]pyrrolidin-1-yl]-2-azaspiro[3.3]heptan-2-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (31 mg, 0.03 mmol, 1.0 eq), 1M TBAF in THE (0.5 mL) and ethylenediamine (26.7 mg, 0.4 mmol, 15 eq) at 25° C. The resulting mixture was stirred for 4 hours at 70° C. The mixture was allowed to cool down to 25° C. The resulting mixture was diluted with water (5 mL). The resulting mixture was extracted with Ethyl acetate (3×5 mL). The combined organic layers were washed with brine (1×5 mL), dried over anhydrous Na₂SO₄. After filtration, the filtrate was concentrated under reduced pressure. The crude residue was purified by reverse flash chromatography with the following conditions: Column, XBridge Shield RP18 OBD Column, 19*150 mm, 5 μm; mobile phase, Water (10 mmol/L NH₄HCO₃ and 0.1% NH₃·H₂O) and CH₃CN (38% CH₃CN up to 54% in 7 min); Detector, UV 220. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{6-[(2S)-2-[2-(prop-1-en-2-yl)phenyl]pyrrolidin-1-yl]-2-azaspiro[3.3]heptan-2-yl}benzamide (12 mg, 44.2%) as light yellow solid. LC-MS (ESI, m/z) M+1: 915. ¹HNMR (300 MHz, DMSO-d₆) δ 12.27 (s, 1H), 11.19-11.01 (m, 1H), 8.33 (d, J=27.1 Hz, 1H), 7.58 (d, J=8.1 Hz, 2H), 7.39 (s, 1H), 7.24 (s, 1H), 7.15 (s, 1H), 7.09 (d, J=12.4 Hz, 1H), 7.01 (d, J=7.7 Hz, 1H), 6.79 (s, 1H), 6.56 (s, 1H), 6.28 (s, 1H), 6.20 (d, J=7.8 Hz, 1H), 6.07 (d, J=10.4 Hz, 1H), 5.99 (s, 1H), 5.21 (s, 1H), 4.72 (s, 1H), 4.64 (s, 1H), 4.43 (d, J=8.6 Hz, 1H), 4.28 (s, 1H), 4.08 (d, J=7.8 Hz, 1H), 3.87 (d, J=11.6 Hz, 3H), 3.80 (s, 1H), 3.73-3.63 (m, 4H), 3.54 (t, J=9.8 Hz, 1H), 3.47 (s, 2H), 3.22 (s, 1H), 3.08 (s, 1H), 2.82 (s, 2H), 2.26 (s, 1H), 2.08 (s, 4H), 2.00 (d, J=5.4 Hz, 3H), 1.78 (s, 1H), 1.71 (d, J=9.0 Hz, 1H), 1.63 (d, J=12.2 Hz, 2H), 1.54 (s, 1H), 1.26 (d, J=15.3 Hz, 4H).

### Example 11: Preparation of 4-[2-[(2S)-2-(2-isopropylphenyl)pyrrolidin-1-yl]-7-azaspiro[3.5]nonan-7-yl]-N-[(3S)-5-nitro-3-(oxan-4-yl)-3,4-dihydro-2H-1,4-benzoxazin-7-ylsulfonyl]-2-[1H-pyrrolo[2,3-b]pyridin-5-yloxy]benzamide and 4-[2-[(2S)-2-(2-isopropylphenyl)pyrrolidin-1-yl]-7-azaspiro[3.5]nonan-7-yl]-N-[(3R)-5-nitro-3-(oxan-4-yl)-3,4-dihydro-2H-1,4-benzoxazin-7-ylsulfonyl]-2-[1H-pyrrolo[2,3-b]pyridin-5-yloxy]benzamide (assumed)

Synthesis of tert-butyl 2-[(2S)-2-[2-(prop-1-en-2-yl)phenyl]pyrrolidin-1-yl]-7-azaspiro[3.5]nonane-7-carboxylate: Into a 250-mL round-bottom flask purged and maintained with an inert atmosphere of nitrogen, were placed tert-butyl 2-[(2S)-2-(2-bromophenyl)pyrrolidin-1-yl]-7-azaspiro[3.5]nonane-7-carboxylate (7.2 g, 15.9 mmol, 1.0 eq), dioxane (72 mL), water (8 mL), 4,4,5,5-tetramethyl-2-(prop-1-en-2-yl)-1,3,2-dioxaborolane (6.4 g, 38.0 mmol, 2.4 eq), K₂CO₃ (6.6 g, 47.8 mmol, 3.0 eq), Pd(dqpf)Cl₂·CH₂Cl₂ (1.3 g, 1.6 mmol, 0.1 eq). The resulting solution was stirred 8 hours at 80° C. in an oil bath. The reaction mixture was cooled to 25° C. The resulting mixture was concentrated under vacuum. The crude residue was purified by a flash column (silica gel, ethyl acetate/petroleum ether=1: 2) to afford tert-butyl 2-[(2S)-2-[2-(prop-1-en-2-yl)phenyl]pyrrolidin-1-yl]-7-azaspiro[3.5]nonane-7-carboxylate (4 g, 66.4%) as yellow oil. LC-MS (ESI, m/z) M+1: 411.

Synthesis of tert-butyl 2-[(2S)-2-(2-isopropylphenyl)pyrrolidin-1-yl]-7-azaspiro[3.5]nonane-7-carboxylate: Into a 100-mL pressure tank reactor (10 atm), were placed tert-butyl 2-[(2S)-2-[2-(prop-1-en-2-yl)phenyl]pyrrolidin-1-yl]-7-azaspiro[3.5]nonane-7-carboxylate (3.6 g, 8.7 mmol, 1.0 eq), EtOH (50 mL), 10% Pd/C (500 mg, 4.7 mmol, 0.5 eq), to the above H₂ was introduced in. The resulting solution was stirred overnight at 50° C. in an oil bath. The reaction mixture was cooled to 25° C. and the solids were filtered out. The resulting mixture was concentrated under vacuum. The crude residue was purified by a flash column (silica gel, ethyl acetate/petroleum ether=1: 2) to afford tert-butyl 2-[(2S)-2-(2-isopropylphenyl)pyrrolidin-1-yl]-7-azaspiro[3.5]nonane-7-carboxylate (3 g, 72.3%) as yellow oil. LC-MS (ESI, m/z) M+1: 413.

Synthesis of 2-[(2S)-2-(2-isopropylphenyl)pyrrolidin-1-yl]-7-azaspiro[3.5]nonane hydrochloride: Into a 100-mL round-bottom flask, were placed tert-butyl 2-[(2S)-2-(2-isopropylphenyl)pyrrolidin-1-yl]-7-azaspiro[3.5]nonane-7-carboxylate (2.6 g, 6.3 mmol, 1.0 eq), HCl (gas) in 1,4-dioxane (20 mL). The resulting solution was stirred for 1 hour at 25° C. The resulting mixture was concentrated under vacuum. This resulted in 2-[(2S)-2-(2-isopropylphenyl)pyrrolidin-1-yl]-7-azaspiro[3.5]nonane hydrochloride (2 g, 95.5%) as a red solid. LC-MS (ESI, m/z) M+1: 313.

Synthesis of methyl 4-[2-[(2S)-2-(2-isopropylphenyl)pyrrolidin-1-yl]-7-azaspiro[3.5]nonan-7-yl]-2-[1H-pyrrolo[2,3-b]pyridin-5-yloxy]benzoate: Into a 40-mL vial, were placed 2-[(2S)-2-(2-isopropylphenyl)pyrrolidin-1-yl]-7-azaspiro[3.5]nonane hydrochloride (700 mg, 2.0 mmol, 1.0 eq), DMSO (10 mL), methyl 4-fluoro-2-[1H-pyrrolo[2,3-b]pyridin-5-yloxy]benzoate (518 mg, 1.8 mmol, 0.9 eq), Na₂CO₃ (533 mg, 5.0 mmol, 2.5 eq). The resulting solution was stirred overnight at 130° C. in an oil bath. The reaction mixture was cooled to 25° C. The resulting solution was diluted with water (200 mL). The resulting solution was extracted with ethyl acetate (3×50 mL) and the organic layer combined. The resulting mixture was washed with brine (1×200 mL). The mixture was dried over anhydrous sodium sulfate. After filtration, the filtrate was concentrated under reduced pressure. The crude residue was purified by a flash column (silica gel, ethyl acetate/petroleum ether=1: 2) to afford methyl 4-[2-[(2S)-2-(2-isopropylphenyl)pyrrolidin-1-yl]-7-azaspiro[3.5]nonan-7-yl]-2-[1H-pyrrolo[2,3-b]pyridin-5-yloxy]benzoate (325 mg, 27.9%) as yellow oil. LC-MS (ESI, m/z) M+1: 579.

Synthesis of 4-[2-[(2S)-2-(2-isopropylphenyl)pyrrolidin-1-yl]-7-azaspiro[3.5]nonan-7-yl]-2-[1H. pyrrolo[2,3-b]pyridin-5-yloxy]benzoic acid: Into a 40-mL vial, were placed methyl 4-[2-[(2S)-2-(2-isopropylphenyl)pyrrolidin-1-yl]-7-azaspiro[3.5]nonan-7-yl]-2-[1H-pyrrolo[2,3-b]pyridin-5-yloxy]benzoate (300 mg, 0.5 mmol, 1.0 eq), MeOH (5 mL), dioxane (5 mL), NaOH (4 M, 2 mL). The resulting solution was stirred for 4 hours at 70° C. in an oil bath. The reaction mixture was cooled to 25° C. The resulting mixture was concentrated under vacuum. The pH value of the solution was adjusted to 6-7 with HCl (2.0 M). The resulting solution was extracted with dichloromethane (3×30 mL) and the organic layers combined. The resulting mixture was washed with brine (1×100 mL). The mixture was dried over anhydrous sodium sulfate. After filtration, the filtrate was concentrated under reduced pressure. The crude residue was purified by a flash column (silica gel, dichloromethane/methanol=10:1) to afford 4-[2-[(2S)-2-(2-isopropylphenyl)pyrrolidin-1-yl]-7-azaspiro[3.5]nonan-7-yl]-2-[1H-pyrrolo[2,3-b]pyridin-5-yloxy]benzoic acid (133 mg, 45.4%) as a light yellow solid. LC-MS (ESI, m/z) M+1: 565.

Synthesis of (2R)-1,4-dioxane-2-carbaldehyde: Into a 100-mL round-bottom flask, were placed (2S)-1,4-dioxan-2-ylmethanol (2.4 g, 20.0 mmol, 1.0 eq), CH₃CN (50 mL), IBX (9.5 g, 40.0 mmol, 1.7 eq). The resulting solution was stirred for 4 hours at 70° C. in an oil bath. The reaction mixture was cooled to 25° C. The solids were filtered out. The resulting mixture was concentrated. This resulted (2R)-1,4-dioxane-2-carbaldehyde (2.0 g, 86.2%) as colorless oil. ¹HNMR (300 MHz, Chloroform-d) δ 9.66 (s, 1H), 4.12-3.77 (m, 7H).

Synthesis of 2-amino-2-[(2S)-1,4-dioxan-2-yl]acetonitrile: Into a 100-mL pressure tank reactor, were placed (2R)-1,4-dioxane-2-carbaldehyde (2.0 g, 17.2 mmol, 1.0 eq), NH₃/MeOH (7 M, 40 mL). This was followed by the addition of TMSCN (3.0 g, 30.0 mmol, 1.7 eq). The resulting solution was stirred for overnight at 70° C. in an oil bath. The resulting mixture was cooled to 25° C. and concentrated. The crude residue was purified by a flash column (silica gel, ethyl acetate/petroleum ether=3:1). This resulted 2-amino-2-[(2S)-1,4-dioxan-2-yl]acetonitrile (2.0 g, 81.7%) as brown oil. LCMS (ESI, m/z) M+1: 143.

Synthesis of amino((2S)-1,4-dioxan-2-yl)acetic acid: Into a 20-mL round-bottom flask, were placed 2-amino-2-[(2S)-1,4-dioxan-2-yl]acetonitrile (2.0 g, 14.1 mmol, 1.0 eq), NaOH (4 M in water) (5 mL). The resulting solution was stirred for overnight at 70° C. in an oil bath. The reaction mixture was cooled to 25° C. and the pH value of the solution was adjusted to 6 with HOAc. The solids were collected by filtration. The solid was dried in an oven under reduced pressure. This resulted amino((2S)-1,4-dioxan-2-yl)acetic acid (1.2 g, 52.9%) as a white solid. LCMS (ESI, m/z) M+1: 162.

Synthesis of 2-amino-2-[(2S)-1,4-dioxan-2-yl]ethanol: Into a 100-mL round-bottom flask, were placed amino((2S)-1,4-dioxan-2-yl)acetic acid (1.2 g, 7.4 mmol, 1.0 eq), THE (40 mL), LiAlH₄ (860 mg, 22.4 mmol, 3.0 eq). The resulting solution was stirred for 4 hours at 60° C. in an oil bath. The reaction mixture was cooled to 25° C. The reaction mixture was quenched by the addition of water (0.86 mL) and 15% NaOH (0.86 mL) and water (2.58 mL) at 0° C. The resulting mixture was filtered, the filter cake was washed with tetrahydrofuran (3×10 mL), dried over anhydrous Na₂SO₄. After filtration, the filtrate was concentrated under reduced pressure. This resulted 2-amino-2-[(2S)-1,4-dioxan-2-yl]ethanol (1.8 g. crude) as colorless oil. LCMS (ESI, m/z) M+1: 148.

Synthesis of 3-bromo-4-([1-[(2S)-1,4-dioxan-2-yl]-2-hydroxyethyl]amino)-5-nitrobenzenesulfonamide: Into a 50-mL round-bottom flask, were placed 3-bromo-4-chloro-5-nitrobenzenesulfonamide (1.2 g, 3.8 mmol, 1.0 eq), 2-amino-2-[(2S)-1,4-dioxan-2-yl]ethanol (72 mg, 4.9 mmol, 1.3 eq), CH₃CN (30 mL), DIEA (1.5 g, 11.4 mmol, 3.0 eq). The resulting solution was stirred for 48 hours at 80° C. in an oil bath. The resulting mixture was cooled to 25° C. and concentrated. The crude residue was purified by a flash column (silica gel, ethyl acetate/petroleum ether=9: 1) to afford (120 mg, 7.5%) 3-bromo-4-([1-[(2S)-1,4-dioxan-2-yl]-2-hydroxyethyl]amino)-5-nitrobenzenesulfonamide as a yellow solid. LCMS (ESI, m/z) M−1: 424.

Synthesis of (3S)-3-[(2S)-1,4-dioxan-2-yl]-5-nitro-3,4-dihydro-2H-1,4-benzoxazine-7-sulfonamide (assumed) and (3R)-3-[(2S)-1,4-dioxan-2-yl]-5-nitro-3,4-dihydro-2H-1,4-benzoxazine-7-sulfonamide Into a 8-mL round-bottom flask purged and maintained with an inert atmosphere of nitrogen, were placed 3-bromo-4-([1-[(2S)-1,4-dioxan-2-yl]-2-hydroxyethyl]amino)-5-nitrobenzenesulfonamide (120 mg, 0.23 mmol, 1.0 eq), dioxane (8 mL), Cs₂CO₃(229 mg, 0.7 mmol, 2.5 eq), t-BuXPhos Pd G3 (22 mg, cat). The resulting solution was stirred for 8 hours at 100° C. in an oil bath. The reaction was cooled to 25° C. and diluted by the addition of water (8 mL). The resulting solution was extracted with ethyl acetate (3×8 mL) and the organic layers combined. The resulting mixture was washed with brine (1×8 mL). The mixture was dried over anhydrous Na₂SO₄. After filtration, the filtrate was concentrated under reduced pressure. The crude product was purified by Prep-HPLC with the following conditions: Column, X-bridge RP18; mobile phase, 0.05% ammonia in water and CH₃CN (45% CH₃CN up to 60% in 5 min); Detector, UV 254 nm. This resulted (7 mg, 7.2%) (3S)-3-[(2S)-1,4-dioxan-2-yl]-5-nitro-3,4-dihydro-2H-1,4-benzoxazine-7-sulfonamide as a yellow solid. This resulted (7 mg, 7.2%) (3R)-3-[(2S)-1,4-dioxan-2-yl]-5-nitro-3,4-dihydro-2H-1,4-benzoxazine-7-sulfonamide as a yellow solid. LC-MS (ESI, m/z) M−1:344.

Synthesis of 4-[2-[(2S)-2-(2-isopropylphenyl)pyrrolidin-1-yl]-7-azaspiro[3.5]nonan-7-yl]-N-[(3S)-5-nitro-3-(oxan-4-yl)-3,4-dihydro-2H-1,4-benzoxazin-7-ylsulfonyl]-2-[1H-pyrrolo[2,3-b]pyridin-5-yloxy]benzamide Into a 20-mL vial, were placed 4-[2-[(2S)-2-(2-isopropylphenyl)pyrrolidin-1-yl]-7-azaspiro[3.5]nonan-7-yl]-2-[1H-pyrrolo[2,3-b]pyridin-5-yloxy]benzoic acid (35 mg, 0.06 mmol, 1.0 eq), CH₂Cl₂ (3 mL), (3S)-5-nitro-3-(oxan-4-yl)-3,4-dihydro-2H-1,4-benzoxazine-7-sulfonamide (22 mg, 0.06 mmol, 1.0 eq), EDCI (24 mg, 0.1 mmol, 2.0 eq), DMAP (30 mg, 0.2 mmol, 4.0 eq). The resulting solution was stirred overnight at 30° C. The resulting mixture was concentrated under vacuum. The crude product was purified by Prep-HPLC with the following conditions (2 #SHIMADZU (HPLC-01)): Column, XBridge Shield RP18 OBD Column, 19*150 mm, 5 um; mobile phase, water (0.05% NH₃·H₂O) and CH₃CN (41% Phase CH₃CN up to 56% in 7 min); Detector, UV. This resulted in 4-[2-[(2S)-2-(2-isopropylphenyl)pyrrolidin-1-yl]-7-azaspiro[3.5]nonan-7-yl]-N-[(3S)-5-nitro-3-(oxan-4-yl)-3,4-dihydro-2H-1,4-benzoxazin-7-ylsulfonyl]-2-[1H-pyrrolo[2,3-b]pyridin-5-yloxy]benzamide (15 mg, 27.2%) as a yellow solid. LC-MS (ESI, m/z) M+1: 890. ¹HNMR (300 MHz, DMSO-d₆) δ 11.59 (s, 1H), 8.72 (s, 1H), 8.14 (s, 1H), 7.99 (s, 1H), 7.52 (d, J=8.5 Hz, 2H), 7.46 (t, J=3.6 Hz, 2H), 7.31 (d, J=2.1 Hz, 1H), 7.23 (s, 1H), 7.15 (s, 2H), 6.69-6.62 (m, 1H), 6.35 (d, J=3.3 Hz, 1H), 6.18 (d, J=2.4 Hz, 1H), 4.30 (d, J=10.8 Hz, 1H), 4.02 (dd, J=11.3, 3.0 Hz, 1H), 3.86 (dd, J=11.4, 4.2 Hz, 2H), 3.70-3.60 (m, 1H), 3.51 (d, J=4.6 Hz, 1H), 3.24 (ddd, J=17.2, 13.1, 8.8 Hz, 2H), 3.01 (t, J=5.7 Hz, 2H), 2.92 (d, J=6.1 Hz, 2H), 2.68 (t, J=2.0 Hz, 1H), 2.33 (t, J=2.0 Hz, 1H), 2.21 (s, 1H), 1.90-1.35 (m, 16H), 1.17 (dd, J=12.8, 6.7 Hz, 6H).

Synthesis of 4-[2-[(2S)-2-(2-isopropylphenyl)pyrrolidin-1-yl]-7-azaspiro[3.5]nonan-7-yl]-N-[(3R)-5-nitro-3-(oxan-4-yl)-3,4-dihydro-2H-1,4-benzoxazin-7-ylsulfonyl]-2-[1H-pyrrolo[2,3-b]pyridin-5-yloxy]benzamide Into a 20-mL vial, were placed 4-[2-[(2S)-2-(2-isopropylphenyl)pyrrolidin-1-yl]-7-azaspiro[3.5]nonan-7-yl]-2-[1H-pyrrolo[2,3-b]pyridin-5-yloxy]benzoic acid (35 mg, 0.06 mmol, 1.0 eq), CH₂Cl₂ (3 mL), (3R)-5-nitro-3-(oxan-4-yl)-3,4-dihydro-2H-1,4-benzoxazine-7-sulfonamide (22 mg, 0.06 mmol, 1.0 eq), EDCI (24 mg, 0.1 mmol, 2.0 eq), DMAP (30 mg, 0.2 mmol, 4.0 eq). The resulting solution was stirred overnight at 30° C. The resulting mixture was concentrated under vacuum. The crude product was purified by Prep-HPLC with the following conditions (2 #SHIMADZU (HPLC-01)): Column, XBridge Shield RP18 OBD Column, 19*150 mm, 5 um; mobile phase, water (0.05% NH₃·H₂O) and CH₃CN (39% CH₃CN up to 54% in 7 min); Detector, UV. This resulted in 4-[2-[(2S)-2-(2-isopropylphenyl)pyrrolidin-1-yl]-7-azaspiro[3.5]nonan-7-yl]-N-[(3R)-5-nitro-3-(oxan-4-yl)-3,4-dihydro-2H-1,4-benzoxazin-7-ylsulfonyl]-2-[1H-pyrrolo[2,3-b]pyridin-5-yloxy]benzamide (20 mg, 36.2%) as a yellow solid. LC-MS (ESI, m/z) M+1: 890. ¹HNMR (300 MHz, DMSO-d₆) δ 11.59 (s, 1H), 8.72 (s, 1H), 8.14 (s, 1H), 7.99 (s, 1H), 7.52 (d, J=8.5 Hz, 2H), 7.46 (t, J=3.6 Hz, 2H), 7.31 (d, J=2.1 Hz, 1H), 7.23 (s, 1H), 7.15 (s, 2H), 6.69-6.62 (m, 1H), 6.35 (d, J=3.3 Hz, 1H), 6.18 (d, J=2.4 Hz, 1H), 4.30 (d, J=10.8 Hz, 1H), 4.02 (dd, J=11.3, 3.0 Hz, 1H), 3.86 (dd, J=11.4, 4.2 Hz, 2H), 3.70-3.60 (m, 1H), 3.51 (d, J=4.6 Hz, 1H), 3.24 (ddd, J=17.2, 13.1, 8.8 Hz, 2H), 3.01 (t, J=5.7 Hz, 2H), 2.92 (d, J=6.1 Hz, 2H), 2.68 (t, J=2.0 Hz, 1H), 2.33 (t, J=2.0 Hz, 1H), 2.21 (s, 1H), 1.90-1.35 (m, 16H), 1.17 (dd, J=12.8, 6.7 Hz, 6H).

### Example 12: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-phenylpyrrolidin-1-yl]-7-azaspiro[3.5]nonan-7-yl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.7 mmol, 1.0 eq), ethyl acetate (20 mL) and 2.0 M HCl in ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.5 mmol, 1.0 eq), N,N-dimethylformamide (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (20 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The residue was purified by a flash column (silica gel, ethyl acetate/petroleum ether=10: 1-2:1) to afford methyl 2-bromo-4-{2-oxo-7-azaspiro[3.5]nonan-7-yl}benzoate (7 g, 46.4%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.4 g, 11.3 mmol, 4.0 eq) at 25° C. The resulting mixture was stirred for 2 hours at 40° C. The mixture was allowed to cool down to 25° C. The resulting mixture was diluted with water (20 mL). The mixture residue was acidified to PH 6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1 g, 72.9%) as light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1 g, 2.957 mmol, 1.00 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The residue was purified by a flash column (silica gel, ethyl acetate/petroleum ether=5:1-0:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃(617 mg, 1.9 mmol, 2.0 eq), copper(I) iodide (36 mg, 0.2 mmol, 0.2 eq), ethanediamide, N,N′-diphenyloxalamide (45 mg, 0.2 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The residue was purified by a flash column (silica gel, ethyl acetate/petroleum ether=10: 1-1:10) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 46.9%) as yellow solid. LC-MS (ESI, m/z) M+1: 902. ¹HNMR (300 MHz, DMSO-d₆): δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-phenylpyrrolidine-1-carboxylate: Into a 8 mL vial were added tert-butyl (2S)-2-(2-bromophenyl)pyrrolidine-1-carboxylate (200 mg, 0.6 mmol, 1.0 eq), N,N-dimethylformamide and Pd(PPh₃)₄ (35 mg, 0.1 mmol, 0.1 eq) at 25° C. The resulting mixture was stirred for 12 hours at 80° C. under nitrogen atmosphere. The mixture was allowed to cool down to 25° C. The resulting mixture was diluted with water (5 mL). The resulting mixture was extracted with Ethyl acetate (3×10 mL). The combined organic layers were washed with brine (10 mL), dried over anhydrous Na₂SO₄. After filtration, the filtrate was concentrated under reduced pressure. The crude residue was purified by a flash column (silica gel, ethyl acetate/petroleum ether=10:1-1:1) to afford tert-butyl (2S)-2-phenylpyrrolidine-1-carboxylate (70 mg, 46.1%) as colorless oil. LC. MS (ESI, m/z) M+1: 192. ¹HNMR (300 MHz, DMSO-d₆): δ 7.31 (dd, J=8.1, 6.6 Hz, 2H), 7.22 (d, J=7.0 Hz, 1H), 7.16 (d, J=7.7 Hz, 2H), 4.76 (d, J=35.2 Hz, 1H), 3.59-3.40 (m, 2H), 2.28 (s, 1H), 1.76 (d, J=33.0 Hz, 3H), 1.40 (s, 4H), 1.09 (s, 5H).

Synthesis of (2S)-2-phenylpyrrolidine: Into a 100 mL round-bottom flask were added tert-butyl (2S)-2-phenylpyrrolidine-1-carboxylate (70 mg, 0.3 mmol, 1.0 eq) and CH₂Cl₂(1.0 mL), TFA (0.5 mL) at 25° C. The resulting mixture was stirred for 3 hours at 25° C. The resulting mixture was concentrated under reduced pressure. This resulted in (2S)-2-phenylpyrrolidine (40 mg, 96.0%) as colorless oil. LC-MS (ESI, m/z) M+1: 148.

Synthesis of N-{3-nitro-4-[(oxan-4-ylmethyl)amino]benzenesulfonyl}-4-{2-[(2S)-2-phenylpyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 8 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1.0 eq) and (2S)-2-phenylpyrrolidine (14 mg, 0.1 mmol, 1.0 eq), CH₂Cl₂ (2.0 mL), NaBH(OAc)₃ (28 mg, 0.1 mmol, 2 eq) at 25° C. The resulting mixture was stirred for 4 hours at 25° C. The reaction was quenched by the addition of MeOH (1.0 mL) at 25° C. The resulting mixture was concentrated under reduced pressure. The crude residue was purified by Prep-TLC (dichloromethane/methanol=95:5) to afford N-{3-nitro-4-[(oxan-4-ylmethyl)amino]benzenesulfonyl}-4-{2-[(2S)-2-phenylpyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (50 mg, 72.7%) as yellow solid. LC-MS (ESI, m/z) M+1: 1034.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-phenylpyrrolidin-1-yl]-7-azaspiro[3.5]nonan-7-yl}benzamide hydrochloride: Into a 8 mL vial were added N-{3-nitro-4-[(oxan-4-ylmethyl)amino]benzenesulfonyl}-4-{2-[(2S)-2-phenylpyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (50 mg, 0.05 mmol, 1.0 eq) and 1 M TBAF in tetrahydrofuran (1.0 mL), ethylenediamine (43 mg, 0.7 mmol, 15 eq) at 25° C. The resulting mixture was stirred for 2 hours at 70° C. The mixture was allowed to cool down to 25° C. The resulting mixture was diluted with water (5 mL). The resulting mixture was extracted with Ethyl acetate (3×5 mL). The combined organic layers were washed with brine (1×5 mL), dried over anhydrous Na₂SO₄. After filtration, the filtrate was concentrated under reduced pressure. The crude product was purified by Chiral-Prep-HPLC with the following conditions (Prep-HPLC-003): Column, SunFire Prep C18 OBD Column, 19*150 mm, 5 μm 10 nm; mobile phase, Water (0.05% TFA) and CH₃CN (30% CH₃CN up to 78% in 8 min); Detector, UV 220 to afford 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-phenylpyrrolidin-1-yl]-7-azaspiro[3.5]nonan-7-yl}benzamide hydrochloride (6 mg, 13.4%) as yellow solid. LC-MS (ESI, m/z) M+1: 903. ¹HNMR (300 MHz, DMSO-d₆): δ 8.50 (dd, J=25.7, 2.2 Hz, 1H), 7.95-7.41 (m, 9H), 7.35-6.73 (m, 3H), 4.81-4.09 (m, 5H), 4.09-3.88 (m, 5H), 3.89-3.40 (m, 10H), 3.37 (d, J=7.0 Hz, 1H), 2.65-2.17 (m, 7H), 2.17-1.68 (m, 9H), 1.68-1.34 (m, 4H).

### Example 13: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(pyridin-3-yl)phenyl]pyrrolidin-1-yl]-7-azaspiro[3.5]nonan-7-yl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), CH₂Cl₂ (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-[2-(pyridin-3-yl)phenyl]pyrrolidine-1-carboxylate: To a stirred solution of tert-butyl (2S)-2-(2-bromophenyl)pyrrolidine-1-carboxylate (1.0 g, 3.1 mmol, 1.0 eq) and pyridin-3-ylboronic acid (1.1 g, 9.2 mmol, 3.0 eq) in 1,4-dioxane (18 mL) and water (2 mL) were added K₂CO₃ (1.3 g, 9.2 mmol, 3.0 eq) and Pd(dqpf)Cl₂·CH₂Cl₂ (251 mg, 0.3 mmol, 0.1 eq). The resulting mixture was stirred for overnight at 80° C. under nitrogen atmosphere. The mixture was allowed to cool down to 25° C. The resulting mixture was concentrated under vacuum. The crude residue was purified by a flash column (silica gel, ethyl acetate/petroleum ether=1:1) to afford tert-butyl (2S)-2-[2-(pyridin-3-yl)phenyl]pyrrolidine-1-carboxylate (300 mg, 30.2%) as a white solid. LC-MS (ESI, m/z) M+1: 325.

Synthesis of 3-{2-[(2S)-pyrrolidin-2-yl]phenyl}pyridine hydrochloride: A solution of tert-butyl (2S)-2-[2-(pyridin-3-yl)phenyl]pyrrolidine-1-carboxylate (300 mg, 0.9 mmol, 1.0 eq) in HCl (gas) in 1,4-dioxane (4 M, 10 mL) was stirred for 1 hour at 25° C. The resulting mixture was concentrated under vacuum to afford 3-{2-[(2S)-pyrrolidin-2-yl]phenyl}pyridine hydrochloride (230 mg, 95.4%) as a white solid. ¹HNMR (300 MHz, Methanol-d₄) δ 9.02 (dd, J=11.1, 3.6 Hz, 2H), 8.72 (d, J=7.8 Hz, 1H), 8.27 (dd, J=8.0, 5.6 Hz, 1H), 7.89 (dd, J=8.0, 1.2 Hz, 1H), 7.75 (td, J=7.7, 1.3 Hz, 1H), 7.64 (td, J=7.5, 1.2 Hz, 1H), 7.49 (dd, J=7.7, 1.4 Hz, 1H), 4.57 (t, J=8.1 Hz, 1H), 3.58-3.49 (m, 1H), 3.47-3.33 (m, 1H), 2.55-2.44 (m, 1H), 2.29 (s, 2H), 2.18 (dt, J=27.8, 7.5 Hz, 1H).

Synthesis of N-{3-nitro-4-[(oxan-4-ylmethyl)amino]benzenesulfonyl}-4-{2-[(2S)-2-[2-(pyridin-3-yl)phenyl]pyrrolidin-1-yl]-7-azaspiro[3.5]nonan-7-yl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1.0 eq) and 3-{2-[(2S)-pyrrolidin-2-yl]phenyl}pyridine hydrochloride (17 mg, 0.1 mmol, 1.0 eq) in CH₂Cl₂ (3 mL) were added NaBH(OAc)₃ (28 mg, 0.1 mmol, 2.0 eq). The resulting mixture was stirred for overnight at 45° C. The resulting mixture was concentrated under vacuum. The residue was purified by Prep-TLC (dichloromethane/methanol=12: 1) to afford N-{3-nitro-4-[(oxan-4-ylmethyl)amino]benzenesulfonyl}-4-{2-[(2S)-2-[2-(pyridin-3-yl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (55 mg, 74.5%) as a yellow solid. LC-MS (ESI, m/z) M+1: 1110.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(pyridin-3-yl)phenyl]pyrrolidin-1-yl]-7-azaspiro[3.5]nonan-7-yl}benzamide hydrochloride: A solution of N-{3-nitro-4-[(oxan-4-ylmethyl)amino]benzenesulfonyl}-4-{2-[(2S)-2-[2-(pyridin-3-yl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (55 mg, 0.1 mmol, 1.0 eq) in TBAF/THF (1.0 M, 5 mL) was added ethylenediamine (60 mg, 0.1 mmol, 20.1 eq). The resulting mixture was stirred for 5 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(pyridin-3-yl)phenyl]pyrrolidin-1-yl]-7-azaspiro[3.5]nonan-7-yl}benzamide hydrochloride (28 mg, 55.6%) as a yellow solid. LC-MS (ESI, m/z) M+1: 980. ¹HNMR (300 MHz, DMSO-d₆) δ 12.33 (s, 1H), 11.76 (s, 1H), 11.14 (d, J=58.4 Hz, 1H), 8.99 (d, J=5.9 Hz, 2H), 8.62-8.39 (m, 3H), 8.31 (d, J=2.3 Hz, 1H), 8.19-8.03 (m, 1H), 7.72-7.29 (m, 5H), 7.17-6.72 (m, 5H), 6.59 (s, 1H), 6.00 (dd, J=3.4, 1.8 Hz, 1H), 4.66-4.43 (m, 1H), 4.31 (q, J=8.9, 8.4 Hz, 2H), 4.16 (d, J=9.2 Hz, 1H), 3.87 (q, J=15.5, 12.2 Hz, 4H), 3.52 (td, J=24.6, 14.9 Hz, 3H), 3.37-3.02 (m, 8H), 2.90 (s, 1H), 2.58 (s, 1H), 2.31-1.79 (m, 6H), 1.71-1.18 (m, 9H).

### Example 14: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(pyridin-4-yl)phenyl]pyrrolidin-1-yl]-7-azaspiro[3.5]nonan-7-yl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), CH₂Cl₂ (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-[2-(pyridin-4-yl)phenyl]pyrrolidine-1-carboxylate: To a stirred solution of tert-butyl (2S)-2-(2-bromophenyl)pyrrolidine-1-carboxylate (500 mg, 1.5 mmol, 1.0 eq) and pyridin-4-ylboronic acid (570 mg, 4.6 mmol, 3.0 eq) in dioxane (9 mL) and water (1 mL) were added K₂CO₃ (640 mg, 4.6 mmol, 3.0 eq) and Pd(dqpf)Cl₂·CH₂Cl₂ (125 mg, 0.1 mmol, 0.1 eq). The resulting mixture was stirred for overnight at 100° C. under nitrogen atmosphere. The mixture was allowed to cool down to 25° C. The resulting mixture was concentrated under vacuum. The crude residue was purified by a flash column (silica gel, ethyl acetate/petroleum ether=1:1) to afford to afford tert-butyl (2S)-2-[2-(pyridin-4-yl)phenyl]pyrrolidine-1-carboxylate (480 mg, 96.5%) as a white solid. LC-MS (ESI, m/z) M+1: 325.

Synthesis of 4-{2-[(2S)-pyrrolidin-2-yl]phenyl}pyridine hydrochloride: A solution of tert-butyl (2S)-2-[2-(pyridin-4-yl)phenyl]pyrrolidine-1-carboxylate (480 mg, 1.5 mmol, 1.0 eq) in HCl (gas) in 1,4-dioxane (10 mL) was stirred for 1 hour at 25° C. The resulting mixture was concentrated under vacuum to afford 4-{2-[(2S)-pyrrolidin-2-yl]phenyl}pyridine hydrochloride (350 mg, 90.7%) as a white solid. ¹HNMR (300 MHz, Methanol-d₄) δ 9.00 (d, J=5.7 Hz, 2H), 8.19 (d, J=5.6 Hz, 2H), 7.91 (d, J=7.9 Hz, 1H), 7.81-7.72 (m, 1H), 7.67 (t, J=7.6 Hz, 1H), 7.55-7.48 (m, 1H), 4.62 (t, J=8.3 Hz, 1H), 3.55 (dd, J=11.3, 7.1 Hz, 1H), 3.43 (dd, J=10.3, 5.8 Hz, 1H), 2.52 (t, J=9.5 Hz, 1H), 2.29 (dq, J=16.9, 8.7 Hz, 2H), 2.15 (t, J=9.7 Hz, 1H).

Synthesis of N-{3-nitro-4-[(oxan-4-ylmethyl)amino]benzenesulfonyl}-4-{2-[(2S)-2-[2-(pyridin-4-yl)phenyl]pyrrolidin-1-yl]-7-azaspiro[3.5]nonan-7-yl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (70 mg, 0.1 mmol, 1.0 eq) and 4-{2-[(2S)-pyrrolidin-2-yl]phenyl}pyridine (18 mg, 0.1 mmol, 1.0 eq) in CH₂Cl₂ (3 mL) was added NaBH(OAc)₃ (33 mg, 0.2 mmol, 2.0 eq). The resulting mixture was stirred for overnight at 45° C. The resulting mixture was concentrated under vacuum. The residue was purified by Prep-TLC (dichloromethane/methanol=12: 1) to afford N-{3-nitro-4-[(oxan-4-ylmethyl)amino]benzenesulfonyl}-4-{2-[(2S)-2-[2-(pyridin-4-yl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (68 mg, 78.9%) as a yellow solid. LC-MS (ESI, m/z) M+1: 1110.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(pyridin-4-yl)phenyl]pyrrolidin-1-yl]-7-azaspiro[3.5]nonan-7-yl}benzamide hydrochloride: A solution of N-{3-nitro-4-[(oxan-4-ylmethyl)amino]benzenesulfonyl}-4-{2-[(2S)-2-[2-(pyridin-4-yl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (68 mg, 0.1 mmol, 1.0 eq) in TBAF/THF (1.0 M, 5 mL) was added ethylenediamine (74 mg, 1.2 mmol, 20.1 eq). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(pyridin-4-yl)phenyl]pyrrolidin-1-yl]-7-azaspiro[3.5]nonan-7-yl}benzamide hydrochloride (23 mg, 36.9%) as a yellow solid. LC-MS (ESI, m/z) M+1: 980. ¹HNMR (300 MHz, DMSO-d₆) δ 12.31 (s, 1H), 11.25 (s, 1H), 8.99 (t, J=5.8 Hz, 2H), 8.61-8.43 (m, 2H), 8.32 (d, J=2.3 Hz, 1H), 8.01-7.92 (m, 2H), 7.76-7.53 (m, 3H), 7.48-7.33 (m, 2H), 7.10 (d, J=14.8 Hz, 1H), 6.98-6.72 (m, 3H), 6.59 (s, 1H), 6.02 (d, J=11.0 Hz, 1H), 4.58 (t, J=8.4 Hz, 1H), 4.33 (t, J=7.9 Hz, 2H), 4.12 (d, J=7.8 Hz, 1H), 4.00-3.78 (m, 4H), 3.71-2.96 (m, 13H), 2.73 (s, 1H), 2.20 (d, J=9.6 Hz, 3H), 1.91 (s, 3H), 1.64 (d, J=13.2 Hz, 2H), 1.31 (d, J=34.3 Hz, 9H).

### Example 15: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(trifluoromethyl)phenyl]pyrrolidin-1-yl]-7-azaspiro[3.5]nonan-7-yl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N₁,N₂-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of (S)-2-methyl-N-{[2-(trifluoromethyl)phenyl]methylidene}propane-2-sulfinamide: To a stirred solution of 2-(trifluoromethyl)benzaldehyde (20.0 g, 114.8 mmol, 1.0 eq) and (S)-2-methylpropane-2-sulfinamide (14.0 g, 115.5 mmol, 1.0 eq) in THE (300 mL) were added Ti(OEt)₄ (52.0 g, 229.7 mmol, 2.0 eq). The resulting mixture was stirred for overnight at 55° C. The mixture was allowed to cool down to 25° C. The resulting mixture was diluted with water and Ethyl acetate (1000/1000 mL). The resulting mixture was filtered, the filter cake was washed with Ethyl acetate and the organic layer was separated. The organic layers were washed with brine (1×500 mL), dried over anhydrous Na₂SO₄. After filtration, the filtrate was concentrated under reduced pressure. This resulted in (S)-2-methyl-N-{[2-(trifluoromethyl)phenyl]methylidene}propane-2-sulfinamide (27 g, 84.1%) as a yellow oil. LC-MS (ESI, m/z) M+1: 278. ¹HNMR (300 MHz, Chloroform-d) δ 8.96 (q, J=2.1 Hz, 1H), 8.25-8.16 (m, 1H), 7.79-7.54 (m, 3H), 1.27 (s, 9H).

Synthesis of (S)—N-[(1S)-3-(1,3-dioxan-2-yl)-1-[2-(trifluoromethyl)phenyl]propyl]-2-methylpropane-2-sulfinamide: Into a 500-mL 3-necked round-bottom flask purged and maintained with an inert atmosphere of nitrogen, were placed Mg (6.9 g, 284.8 mmol, 4.0 eq), I₂ (1.8 g, 7.2 mmol, 0.1 eq), THE (300 mL), the resulting solution was warmed to 65° C., followed by the addition of 2-(2-bromoethyl)-1,3-dioxane (5.6 g, 28.9 mmol, 0.4 eq) in THE (50 mL) at 65° C. After addition completed, the mixture was cooled to 25° C., another 2-(2-bromoethyl)-1,3-dioxane (50.4 g, 259.9 mmol, 3.6 eq) in THE (250 mL) was added. The resulting solution was cooled to −40° C. in a liquid nitrogen bath. (S)-2-methyl-N-{[2-(trifluoromethyl)phenyl]methylidene}propane-2-sulfinamide (20.0 g, 72.1 mmol, 1.0 eq) in CH₂Cl₂ (50 mL) was added dropwise at −40° C. under nitrogen atmosphere. The resulting solution was stirred for 1 hour at −40° C. in a liquid nitrogen bath. The reaction was then quenched by the addition of aqueous NH₄Cl (1000 mL). The resulting solution was extracted with dichloromethane (3×500 mL) and the organic layers combined. The resulting mixture was washed with brine (1×2000 mL). The mixture was dried over anhydrous sodium sulfate. After filtration, the filtrate was concentrated under reduced pressure. This resulted in (S)—N-[(1S)-3-(1,3-dioxan-2-yl)-1-[2-(trifluoromethyl)phenyl]propyl]-2-methylpropane-2-sulfinamide (26 g, 91.6%) as a yellow oil. LC-MS (ESI, m/z) M+1: 394.

Synthesis of (2S)-2-[2-(trifluoromethyl)phenyl]pyrrolidine: Into a 500-mL round-bottom flask, were placed (S)—N-[(1S)-3-(1,3-dioxan-2-yl)-1-[2-(trifluoromethyl)phenyl]propyl]-2-methylpropane-2-sulfinamide (10.0 g, 25.4 mmol, 1.0 eq), TFA (100 mL), water (25 mL), the resulting solution was stirred for 1 hour at 25° C. TFA (50 mL), Et₃SiH (8.9 g, 76.5 mmol, 3.0 eq) was added. The resulting solution was stirred overnight at 25° C. The resulting mixture was concentrated. The resulting solution was diluted with water (500 mL). The resulting solution was extracted with MTBE (2×200 mL) and the organic layers combined. The resulting mixture was washed with 2 N HCl (1×500 mL). The pH value of the aqueous was adjusted to 13 with NaOH (4 mol/L). The resulting solution was extracted with dichloromethane (3×500 mL) and the organic layers combined and dried over anhydrous sodium sulfate. After filtration, the filtrate was concentrated under reduced pressure. This resulted in (2S)-2-[2-(trifluoromethyl)phenyl]pyrrolidine (1.8 g, 32.9%) as a yellow oil. LC-MS (ESI, m/z) M+1: 216. ¹HNMR (300 MHz, DMSO-d₆) δ 7.93 (d, J=7.9 Hz, 1H), 7.61 (d, J=7.7 Hz, 2H), 7.39 (t, J=7.7 Hz, 1H), 4.43-4.34 (m, 1H), 3.09 (ddd, J=9.6, 7.3, 5.1 Hz, 1H), 2.94 (dt, J=9.6, 7.4 Hz, 1H), 2.20-2.07 (m, 1H), 1.89-1.65 (m, 2H), 1.39 (dq, J=15.9, 7.9 Hz, 1H).

Synthesis of N-{3-nitro-4-[(oxan-4-ylmethyl)amino]benzenesulfonyl}-4-{2-[(2S)-2-[2-(trifluoromethyl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.00 eq) and (2S)-2-[2-(trifluoromethyl)phenyl]pyrrolidine (24 mg, 0.1 mmol, 1.0 eq) in CH₂Cl₂ (3 mL) were added NaBH(OAc)₃ (47 mg, 0.2 mmol, 2.0 eq). The resulting mixture was stirred for overnight at 25° C. The residue was purified by Prep-TLC (dichloromethane/methanol=12: 1) to afford N-{3-nitro-4-[(oxan-4-ylmethyl)amino]benzenesulfonyl}-4-{2-[(2S)-2-[2-(trifluoromethyl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 81.9%) as a yellow solid. LC-MS (ESI, m/z) M+1: 1101.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(trifluoromethyl)phenyl]pyrrolidin-1-yl]-7-azaspiro[3.5]nonan-7-yl}benzamide hydrochloride: Into a 100 mL round-bottom flask were added N-{3-nitro-4-[(oxan-4-ylmethyl)amino]benzenesulfonyl}-4-{2-[(2S)-2-[2-(trifluoromethyl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq), TBAF/THF (1.0 M, 5 mL), ethylenediamine (109 mg, 1.8 mmol, 20.0 eq). The resulting mixture was stirred for 5 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(trifluoromethyl)phenyl]pyrrolidin-1-yl]-7-azaspiro[3.5]nonan-7-yl}benzamide hydrochloride (20 mg, 21.8%) as a yellow solid. LC-MS (ESI, m/z) M+1: 971. ¹HNMR (300 MHz, DMSO-d₆) δ 12.30 (s, 1H), 11.97 (d, J=55.9 Hz, 1H), 11.12 (d, J=84.9 Hz, 1H), 8.79-8.38 (m, 2H), 8.32 (s, 1H), 7.89-7.72 (m, 2H), 7.62 (d, J=8.8 Hz, 2H), 7.54-7.00 (m, 3H), 6.95-6.67 (m, 3H), 6.58 (s, 1H), 6.01 (d, J=15.6 Hz, 1H), 4.64-4.45 (m, 2H), 4.31 (d, J=9.3 Hz, 2H), 3.85 (dt, J=28.8, 14.0 Hz, 6H), 3.63-2.93 (m, 11H), 2.41 (s, 2H), 2.01 (d, J=94.0 Hz, 6H), 1.63 (d, J=13.0 Hz, 2H), 1.46 (s, 7H).

### Example 16: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methoxyphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of (2S)-2-(2-methoxyphenyl)pyrrolidine hydrochloride: To a stirred solution of (2S)-2-(2-bromophenyl)pyrrolidine (300 mg, 1.3 mmol, 1.0 eq) in MeOH (5 mL) were added CuBr (95 mg, 0.6 mmol, 0.5 eq) and MeONa (216 mg, 3.9 mmol, 3.0 eq). The resulting mixture was stirred for overnight at 80° C. under nitrogen atmosphere. The mixture was allowed to cool down to 25° C. The resulting mixture was concentrated under vacuum. The crude residue was purified by a flash column (silica gel, dichloromethane/methanol=10: 1), then purified with Prep-HPLC to afford (2S)-2-(2-methoxyphenyl)pyrrolidine hydrochloride (100 mg, 35.3%) as a white solid. LC-MS (ESI, m/z) M+1: 178. ¹HNMR (300 MHz, Methanol-d₄) δ 7.52-7.40 (m, 1H), 7.41 (dd, J=7.6, 1.7 Hz, 1H), 7.19-7.10 (m, 1H), 7.05 (td, J=7.5, 1.1 Hz, 1H), 4.83-4.72 (m, 1H), 3.96 (s, 3H), 3.44 (dq, J=16.1, 7.7 Hz, 2H), 2.50-2.29 (m, 2H), 2.35-2.19 (m, 1H), 2.24-2.05 (m, 1H).

Synthesis of 4-{2-[(2S)-2-(2-methoxyphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (84 mg, 0.1 mmol, 1.0 eq) and (2S)-2-(2-methoxyphenyl)pyrrolidine hydrochloride (20 mg, 0.1 mmol, 1.0 eq) in CH₂Cl₂ (3 mL) were added NaBH(OAc)₃ (40 mg, 0.2 mmol, 2.0 eq). The resulting mixture was stirred for overnight at 25° C. The resulting mixture was concentrated under vacuum. The residue was purified by Prep-TLC (dichloromethane/methanol=13: 1) to afford 4-{2-[(2S)-2-(2-methoxyphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (80 mg, 80.4%) as a yellow solid. LC-MS (ESI, m/z) M+1: 1063.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methoxyphenyl)pyrrolidin-1-yl]-7-azaspiro[3.5]nonan-7-yl}-N-{3-nitro-4-[(oxan-4-ylmethyl)amino]benzenesulfonyl}benzamide: To a stirred solution of 4-{2-[(2S)-2-(2-methoxyphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (80 mg, 0.1 mmol, 1.0 eq) in TBAF/THF (1.0 M, 3 mL) were added ethylenediamine (90 mg, 1.5 mmol, 20.0 eq). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% NH₃·H₂O) and CH₃CN (35% CH₃CN up to 75% in 10 min); Detector, uv. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methoxyphenyl)pyrrolidin-1-yl]-7-azaspiro[3.5]nonan-7-yl}-N-{3-nitro-4-[(oxan-4-ylmethyl)amino]benzenesulfonyl}benzamide (24 mg, 34.2%) as a yellow solid. LC-MS (ESI, m/z) M+1: 933. ¹HNMR (300 MHz, DMSO-d₆) δ 11.07 (d, J=34.7 Hz, 1H), 8.47-8.25 (m, 2H), 7.54 (dd, J=29.4, 8.1 Hz, 2H), 7.37-6.49 (m, 9H), 6.02 (dt, J=15.2, 2.6 Hz, 1H), 4.68 (s, 1H), 4.52-4.39 (m, 1H), 4.29-4.03 (m, 2H), 3.97-3.68 (m, 7H), 3.62-3.39 (m, 2H), 3.29-2.92 (m, 8H), 2.33-1.14 (m, 16H).

### Example 17: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-[2-(methylamino)phenyl]pyrrolidin-1-yl]-7-azaspiro[3.5]nonan-7-yl}-N-{3-nitro-4-[(oxan-4-ylmethyl)amino]benzenesulfonyl}benzamide

Synthesis of 7-azaspiro[3.5]nonan-2-one hydrochloride Into a 500 mL 3-necked round-bottom flask were added tert-butyl 2-oxo-7-azaspiro[3.5]nonane-7-carboxylate (10 g, 41.78 mmol, 1.00 eq), ethyl acetate (20 mL) and 2.0 M HCl in ethyl acetate (80 mL) at 25° C. The resulting mixture was stirred for 4 h at 25° C. The resulting mixture was concentrated under reduced pressure. This resulted in 7-azaspiro[3.5]nonan-2-one hydrochloride (7.3 g, 99.45%) as white solid. ¹HNMR (300 MHz, DMSO-d) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.55 mmol, 1.00 eq), DMF (100 mL), Cs₂CO₃ (33.85 g, 103.89 mmol, 2.5 eq) and methyl 2-bromo-4-fluorobenzoate (10.5 g, 45.05 mmol, 1.08 eq) at 25° C. The resulting mixture was stirred for 8 h at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The residue was purified by silica gel column chromatography, eluted with petroleum ether/ethyl acetate (10:01-2:1) to afford methyl 2-bromo-4-{2-oxo-7-azaspiro[3.5]nonan-7-yl}benzoate (6.8 g, 46.46%) as light yellow solid. LC-MS (ESI, m/z) M+1: 352; ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1 g, 2.83 mmol, 1.00 eq) and MeOH (5 mL), water (5 mL), NaOH (0.45 g, 11.35 mmol, 4 eq) at room temperature. The resulting mixture was stirred for 2 h at 40° C. The mixture was allowed to cool down to 25° C. The resulting mixture was diluted with water (20 mL). The mixture/residue was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.90%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1 g, 2.95 mmol, 1.00 eq) and 3-nitro-4-[(oxan-4-ylmethyl)amino]benzenesulfonamide (1.03 g, 3.25 mmol, 1.1 eq), DMAP (0.72 g, 5.91 mmol, 2.0 eq), DCM (10 mL), EDCI (0.68 g, 3.54 mmol, 1.2 eq) at 25° C. The resulting mixture was stirred for 3 h at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with petroleum ether/ethyl acetate (5:1-0:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.93%) as yellow solid. LC-MS (ESI, m/z) M+1: 635/337. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90 3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77 1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.94 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328.06 mg, 0.944 mmol, 1.0 eq), Cs₂CO₃ (617.11 mg, 1.888 mmol, 2 eq), copper(I) iodide (35.96 mg, 0.189 mmol, 0.2 eq), N₁,N₂-diphenyloxalamide (45.37 mg, 0.189 mmol, 0.2 eq), dimethylformamide (6 mL) at 25° C. The resulting mixture was stirred for 2 h at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The residue was purified by silica gel column chromatography, eluted with (Ethyl acetate/DCM=1:1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 46.97%) as yellow solid. LC-MS (ESI, m/z) M+1: 902; ¹H NMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17 g, 75.18 mmol, 1.0 eq), DCM (200 mL), Boc₂O (25 g, 114.54 mmol, 1.5 eq), TEA (15.3 g, 151.20 mmol, 2.0 eq), DMAP (922 mg, 7.54 mmol, 0.10 eq). The resulting solution was stirred overnight at 25° C. The resulting mixture was concentrated under vacuum. The residue was applied onto a silica gel column with petroleum ether/ethyl acetate (15: 1). This resulted in tert-butyl (2S)-2-(2-bromophenyl)pyrrolidine-1-carboxylate (24.4 g, 99.48%) as a white solid. LC-MS (ESI, m/z) M-t-Bu+CH₃CN: 311. ¹HNMR (400 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-[2-(methylamino)phenyl]pyrrolidine-1-carboxylate: Into a 50 mL pressure tank reactor were added tert-butyl (2S)-2-(2-bromophenyl)pyrrolidine-1-carboxylate (1 g, 3.06 mmol, 1.0 eq) and methylamine water solution (20 mL, 643.97 mmol, 210.0 eq), Cu (2.0 g, 31.47 mmol, 10.27 eq) and Cu₂O (4.5 g, 31.44 mmol, 10.2 eq). The resulting mixture was stirred for 4 days at 100° C. The mixture was allowed to cool down to 25° C. The resulting mixture was diluted with water (200 mL). The resulting mixture was extracted with CH₂Cl₂ (2×50 mL). The combined organic layers were washed with brine (1×200 mL), dried over anhydrous Na₂SO₄. After filtration, the filtrate was concentrated under reduced pressure. The residue was purified by silica gel column chromatography, eluted with petroleum ether/ethyl acetate (3: 1) to afford tert-butyl (2S)-2-[2-(methylamino)phenyl]pyrrolidine-1-carboxylate (300 mg, 35.41%) as a colorless oil. LC-MS (ESI, m/z) M+1: 277.

Synthesis of N-methyl-2-[(2S)-pyrrolidin-2-yl]aniline: To a stirred solution of tert-butyl (2S)-2-[2-(methylamino)phenyl]pyrrolidine-1-carboxylate (300 mg, 1.085 mmol, 1.00 eq) in DCM (4 mL) were added TFA (0.5 mL). The resulting mixture was stirred for overnight at 25° C. The mixture was basified to pH 10 with saturated Na₂CO₃ (aq.). The aqueous layer was extracted with CH₂Cl₂ (2×50 mL). The resulting mixture was washed with 1×100 mL of brine. The resulting mixture was dried over anhydrous Na₂SO₄. After filtration, the filtrate was concentrated under reduced pressure. This resulted in N-methyl-2-[(2S)-pyrrolidin-2-yl]aniline (123 mg, 64.29%) as a yellow oil. LC-MS (ESI, m/z) M+1: 177; ¹HNMR (300 MHz, DMSO-d₆) δ 7.05 (ddd, J=7.5, 6.2, 1.8 Hz, 2H), 6.58-6.44 (m, 2H), 3.91 (dd, J=8.6, 6.9 Hz, 1H), 3.31 (s, 1H), 3.09-2.96 (m, 1H), 2.89-2.75 (m, 1H), 2.03-1.87 (m, 1H), 1.84-1.60 (m, 3H).

Synthesis of 4-{2-[(2S)-2-[2-(methylamino)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (80 mg, 0.09 mmol, 1.0 eq) and N-methyl-2-[(2S)-pyrrolidin-2-yl]aniline (16 mg, 0.09 mmol, 1.02 eq) in DCM (3 mL, 47.19 mmol, 532.1 eq) were added NaBH(OAc)₃ (38 mg, 0.179 mmol, 2.0 eq). The resulting mixture was stirred for overnight at 25° C. The residue was purified by Prep-TLC (CH₂Cl₂/MeOH=13: 1) to afford 4-{2-[(2S)-2-[2-(methylamino)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (78 mg, 82.79%) as a yellow solid. LC-MS (ESI, m/z) M+1: 1062.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-[2-(methylamino)phenyl]pyrrolidin-1-yl]-7-azaspiro[3.5]nonan-7-y}-N-{3-nitro-4-[(oxan-4-ylmethyl)amino]benzenesulfonyl}benzamide: Into a 100 mL round-bottom flask were added 4-{2-[(2S)-2-[2-(methylamino)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (73 mg, 0.07 mmol, 1.0 eq) and ethylenediamine (83 mg, 1.381 mmol, 20.1 eq) in TBAF/THF (1.0 M, 5 mL). The resulting mixture was stirred for 3 h at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, Water (0.05% NH₃·H₂O) and CH₃CN (35% ACN up to 75% in 10 min); Detector, uv. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-[2-(methylamino)phenyl]pyrrolidin-1-yl]-7-azaspiro[3.5]nonan-7-yl}-N-{3-nitro-4-[(oxan-4-ylmethyl)amino]benzenesulfonyl}benzamide (7 mg, 10.93%) as a yellow solid. LC-MS (ESI, m/z) M+1: 932. ¹HNMR (300 MHz, DMSO-d₆) δ 11.10 (d, J=55.4 Hz, 1H), 8.50 (s, 1H), 8.34 (d, J=18.9 Hz, 1H), 7.55 (d, J=9.3 Hz, 1H), 7.37 (s, 1H), 7.08 (d, J=12.7 Hz, 2H), 6.87 (d, J=48.7 Hz, 3H), 6.68-6.43 (m, 4H), 6.02 (d, J=13.9 Hz, 1H), 4.63 (s, 1H), 4.46 (d, J=8.7 Hz, 1H), 4.29 (s, 1H), 4.10 (d, J=6.8 Hz, 1H), 3.87 (d, J=11.8 Hz, 4H), 3.65-3.46 (m, 2H), 3.27 2.84 (m, 10H), 2.74 (s, 3H), 2.32-1.56 (m, 11H), 1.35 (dd, J=49.1, 19.3 Hz, 9H).

Synthesis of tert-butyl (2S)-2-[2-(methylamino)phenyl]pyrrolidine-1-carboxylate: Into a 50 mL pressure tank reactor were added tert-butyl (2S)-2-(2-bromophenyl)pyrrolidine-1-carboxylate (1.0 g, 3.1 mmol, 1.0 eq) and methylamine water solution (20 mL), Cu (2.0 g, 31.5 mmol, 10.3 eq) and Cu₂O (4.5 g, 31.4 mmol, 10.3 eq). The resulting mixture was stirred for 4 days at 100° C. The mixture was allowed to cool down to 25° C. The resulting mixture was diluted with water (200 mL). The resulting mixture was extracted with dichloromethane (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1: 3) to afford tert-butyl (2S)-2-[2-(methylamino)phenyl]pyrrolidine-1-carboxylate (300 mg, 35.4%) as a colorless oil. LC-MS (ESI, m/z) M+1: 277.

Synthesis of N-methyl-2-[(2S)-pyrrolidin-2-yl]aniline: To a stirred solution of tert-butyl (2S)-2-[2-(methylamino)phenyl]pyrrolidine-1-carboxylate (300 mg, 1.1 mmol, 1.0 eq) in CH₂Cl₂ (8 mL) were added TFA (1 mL). The resulting mixture was stirred for overnight at 25° C. The mixture was basified to pH 10 with saturated Na₂CO₃ (aq.). The aqueous layer was extracted with dichloromethane (2×50 mL). The resulting mixture was washed with brine (1×100 mL). The resulting mixture was dried over anhydrous Na₂SO₄. After filtration, the filtrate was concentrated under reduced pressure. This resulted in N-methyl-2-[(2S)-pyrrolidin-2-yl]aniline (123 mg, 64.3%) as a yellow oil. LC-MS (ESI, m/z) M+1: 177. ¹HNMR (300 MHz, DMSO-d₆) δ 7.05 (ddd, J=7.5, 6.2, 1.8 Hz, 2H), 6.58-6.44 (m, 2H), 3.91 (dd, J=8.6, 6.9 Hz, 1H), 3.31 (s, 1H), 3.09-2.96 (m, 1H), 2.89-2.75 (m, 1H), 2.03-1.87 (m, 1H), 1.84-1.60 (m, 3H).

Synthesis of 4-{2-[(2S)-2-[2-(methylamino)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (80 mg, 0.1 mmol, 1.0 eq) and N-methyl-2-[(2S)-pyrrolidin-2-yl]aniline (16 mg, 0.1 mmol, 1.0 eq) in CH₂Cl₂ (3 mL) were added NaBH(OAc)₃ (38 mg, 0.2 mmol, 2.0 eq). The resulting mixture was stirred for overnight at 25° C. The residue was purified by Prep-TLC (dichloromethane/methanol=13: 1) to afford 4-{2-[(2S)-2-[2-(methylamino)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (78 mg, 82.8%) as a yellow solid. LC-MS (ESI, m/z) M+1: 1062.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-[2-(methylamino)phenyl]pyrrolidin-1-yl]-7-azaspiro[3.5]nonan-7-yl}. N-{3-nitro-4-[(oxan-4-ylmethyl)amino]benzenesulfonyl}benzamide: Into a 100 mL round-bottom flask were added 4-{2-[(2S)-2-[2-(methylamino)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (73 mg, 0.1 mmol, 1.0 eq) and ethylenediamine (83 mg, 1.4 mmol, 20.1 eq) in TBAF/THF (1.0 M, 5 mL). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% NH₃·H₂O) and CH₃CN (35% CH₃CN up to 75% in 10 min); Detector, uv. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-y]-4-{2-[(2S)-2-[2-(methylamino)phenyl]pyrrolidin-1-yl]-7-azaspiro[3.5]nonan-7-yl}-N-{3-nitro-4-[(oxan-4-ylmethyl)amino]benzenesulfonyl}benzamide (7 mg, 10.9%) as a yellow solid. LC-MS (ESI, m/z) M+1: 932. ¹HNMR (300 MHz, DMSO-d₆) δ 11.10 (d, J=55.4 Hz, 1H), 8.50 (s, 1H), 8.34 (d, J=18.9 Hz, 1H), 7.55 (d, J=9.3 Hz, 1H), 7.37 (s, 1H), 7.08 (d, J=12.7 Hz, 2H), 6.87 (d, J=48.7 Hz, 3H), 6.68-6.43 (m, 4H), 6.02 (d, J=13.9 Hz, 1H), 4.63 (s, 1H), 4.46 (d, J=8.7 Hz, 1H), 4.29 (s, 1H), 4.10 (d, J=6.8 Hz, 1H), 3.87 (d, J=11.8 Hz, 4H), 3.65-3.46 (m, 2H), 3.27-2.84 (m, 10H), 2.74 (s, 3H), 2.32-1.56 (m, 11H), 1.35 (dd, J=9.1, 19.3 Hz, 9H).

### Example 18: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(pyridin-2-yl)phenyl]pyrrolidin-1-yl]-7-azaspiro[3.5]nonan-7-yl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), CH₂Cl₂ (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-[2-(pyridin-2-yl)phenyl]pyrrolidine-1-carboxylate: To a stirred solution of tert-butyl (2S)-2-(2-bromophenyl)pyrrolidine-1-carboxylate (500 mg, 1.5 mmol, 1.0 eq) and 2-(tributylstannyl)pyridine (850 mg, 2.3 mmol, 1.5 eq) in toluene (10 mL) were added Pd(PPh₃)₄ (178 mg, 0.1 mmol, 0.1 eq). The resulting mixture was stirred for overnight at 100° C. under nitrogen atmosphere. The mixture was allowed to cool down to 25° C. The resulting mixture was concentrated under vacuum. The crude residue was purified by a flash column (silica gel, ethyl acetate/petroleum ether=1: 3) to afford tert-butyl (2S)-2-[2-(pyridin-2-yl)phenyl]pyrrolidine-1-carboxylate (270 mg, 54.3%) as a white solid. LC-MS (ESI, m/z) M+1: 325.

Synthesis of 2-{2-[(2S)-pyrrolidin-2-yl]phenyl}pyridine hydrochloride: A solution of tert-butyl (2S)-2-[2-(pyridin-2-yl)phenyl]pyrrolidine-1-carboxylate (270 mg, 0.8 mmol, 1.0 eq) in HCl (gas) in 1,4-dioxane (4 M, 5 mL) was stirred for 1 hour. The resulting mixture was concentrated under vacuum. This resulted in 2-{2-[(2S)-pyrrolidin-2-yl]phenyl}pyridine hydrochloride (210 mg, 96.7%) as a white solid. LC-MS (ESI, m/z) M+1: 225. ¹HNMR (300 MHz, Methanol-d₄) δ 9.03-8.96 (m, 1H), 8.75 (td, J=7.9, 1.5 Hz, 1H), 8.28-8.12 (m, 2H), 7.99-7.91 (m, 1H), 7.86 (td, J=7.6, 1.5 Hz, 1H), 7.73 (td, J=7.5, 1.3 Hz, 1H), 7.66 (dd, J=7.8, 1.5 Hz, 1H), 4.57 (dd, J=9.5, 7.0 Hz, 1H), 3.57 (dt, J=11.4, 8.2 Hz, 1H), 3.49-3.37 (m, 1H), 2.61-2.44 (m, 1H), 2.39-2.07 (m, 3H).

Synthesis of N-{3-nitro-4-[(oxan-4-ylmethyl)amino]benzenesulfonyl}-4-{2-[(2S)-2-[2-(pyridin-2-yl)phenyl]pyrrolidin-1-yl]-7-azaspiro[3.5]nonan-7-yl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (78 mg, 0.1 mmol, 1.0 eq) and 2-{2-[(2S)-pyrrolidin-2-yl]phenyl}pyridine hydrochloride (23 mg, 0.1 mmol, 1.0 eq) in CH₂Cl₂ (3 mL) were added NaBH(OAc)₃ (74 mg, 0.4 mmol, 4.0 eq). The resulting mixture was stirred for overnight at 45° C. The mixture was allowed to cool down to 25° C. The resulting mixture was concentrated under vacuum. The residue was purified by Prep-TLC (dichloromethane/methanol=13: 1) to afford N-{3-nitro-4-[(oxan-4-ylmethyl)amino]benzenesulfonyl}-4-{2-[(2S)-2-[2-(pyridin-2-yl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (77 mg, 80.2%) as a yellow solid. LC-MS (ESI, m/z) M+1: 1110.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(pyridin-2-yl)phenyl]pyrrolidin-1-yl]-7-azaspiro[3.5]nonan-7-yl}benzamide: To a stirred solution of N-{3-nitro-4-[(oxan-4-ylmethyl)amino]benzenesulfonyl}-4-{2-[(2S)-2-[2-(pyridin-2-yl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (77 mg, 0.1 mmol, 1.0 eq) in TBAF/THF (1.0 M, 5 mL) were added ethylenediamine (84 mg, 1.4 mmol, 20.1 eq). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% NH₃·H₂O) and CH₃CN (35% CH₃CN up to 75% in 10 min); Detector, uv. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(pyridin-2-yl)phenyl]pyrrolidin-1-yl]-7-azaspiro[3.5]nonan-7-yl}benzamide (17 mg, 25.0%) as a yellow solid. LC-MS (ESI, m/z) M+1: 980. ¹HNMR (300 MHz, DMSO-d₆) δ 11.10 (d, J=49.7 Hz, 1H), 8.80-8.25 (m, 3H), 8.02-7.03 (m, 10H), 6.95-6.49 (m, 4H), 6.06-5.96 (m, 1H), 4.64 (s, 1H), 4.46 (d, J=8.5 Hz, 1H), 4.28 (s, 1H), 4.10 (d, J=7.4 Hz, 1H), 3.85 (t, J=12.0 Hz, 4H), 3.62-3.36 (m, 3H), 3.29 (d, J=9.7 Hz, 3H), 3.04 (t, J=24.9 Hz, 5H), 2.37-2.03 (m, 2H), 1.96-1.16 (m, 17H).

### Example 19: Preparation of 4-{2-[(2S)-2-[2-(1-cyano-1-methylethyl)phenyl]pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N₁,N₂-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), CH₂Cl₂ (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-[2-(cyanomethyl)phenyl]pyrrolidine-1-carboxylate: Into a 40 mL vial were added tert-butyl (2S)-2-(2-bromophenyl)pyrrolidine-1-carboxylate (1.0 g, 3.1 mmol, 1.0 eq), sodium 2-cyanoacetate (1.1 g, 10.6 mmol, 3.5 eq), mesitylene (10 mL), s-phos (253 mg, 0.6 mmol, 0.2 eq), allylpalladium chloride dimer (112 mg, 0.3 mmol, 0.1 eq). The resulting mixture was stirred for overnight at 140° C. under nitrogen atmosphere. The mixture was allowed to cool down to 25° C. The crude residue was purified by a flash column (silica gel, ethyl acetate/petroleum ether=1: 3) to afford to afford tert-butyl (2S)-2-[2-(cyanomethyl)phenyl]pyrrolidine-1-carboxylate (590 mg, 67.2%) as a yellow oil. LC-MS (ESI, m/z) M-t-Bu+CH₃CN: 272. ¹HNMR (400 MHz, Chloroform-d) δ 7.43-7.30 (m, 2H), 7.28-7.12 (m, 2H), 4.95 (d, J=32.9 Hz, 1H), 4.04-3.49 (m, 4H), 2.47 (s, 1H), 1.98 (dtq, J=32.3, 13.1, 6.9 Hz, 2H), 1.80 (dt, J=12.5, 6.0 Hz, 1H), 1.45 (s, 3H), 1.16 (s, 6H).

Synthesis of tert-butyl (2S)-2-[2-(1-cyano-1-methylethyl)phenyl]pyrrolidine-1-carboxylate: To a solution of tert-butyl (2S)-2-[2-(cyanomethyl)phenyl]pyrrolidine-1-carboxylate (530 mg, 1.8 mmol, 1.0 eq) in THE (10 mL) was added 2.0 M NaHMDS (2.1 mL, 4.3 mmol, 2.3 eq) at 0° C. The mixture was stirred for 30 min. CH₃1 (632 mg, 4.5 mmol, 2.4 eq) was added and the mixture was allowed to warm to 25° C. and stirred for 4 hours. The reaction mixture was quenched by NH₄Cl (aq) and extracted with Ethyl acetate (3×50 mL). The resulting mixture was washed with brine (1×200 mL), dried over anhydrous Na₂SO₄. After filtration, the filtrate was concentrated under reduced pressure. The crude product was purified by Flash-Prep-HPLC with the following conditions (95% CH₃CN in water (0.05% NH₃·H₂O)) to afford tert-butyl (2S)-2-[2-(1-cyano-1-methylethyl)phenyl]pyrrolidine-1-carboxylate (248 mg, 42.6%) as a yellow oil. LC-MS (ESI, m/z) M-t-Bu+CH₃CN: 300.

Synthesis of 2-methyl-2-{2-[(2S)-pyrrolidin-2-yl]phenyl}propanenitrile hydrochloride: A solution of tert-butyl (2S)-2-[2-(1-cyano-1-methylethyl)phenyl]pyrrolidine-1-carboxylate (230 mg, 0.7 mmol, 1.0 eq) in HCl (gas) in 1,4-dioxane (5 mL) was stirred for 1 hour at 25° C. The resulting mixture was concentrated under vacuum. This resulted in 2-methyl-2-{2-[(2S)-pyrrolidin-2-yl]phenyl}propanenitrile hydrochloride (160 mg, 87.2%) as a yellow oil. LC-MS (ESI, m/z) M+1: 215. ¹HNMR (400 MHz, Methanol-d₄) δ 7.71 (dd, J=8.0, 1.7 Hz, 1H), 7.63-7.47 (m, 3H), 5.39 (t, J=8.1 Hz, 1H), 3.67-3.51 (m, 2H), 2.79 (tt, J=9.7, 5.6 Hz, 1H), 2.51-2.32 (m, 1H), 2.32-2.07 (m, 2H), 1.88 (d, J=1.2 Hz, 6H).

Synthesis of 4-{2-[(2S)-2-[2-(1-cyano-1-methylethyl)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added 2-methyl-2-{2-[(2S)-pyrrolidin-2-yl]phenyl}propanenitrile hydrochloride (28 mg, 0.1 mmol, 1.0 eq), CH₂Cl₂ (3 mL), N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq), NaBH(OAc)₃ (71 mg, 0.3 mmol, 3.0 eq). The resulting mixture was stirred for overnight at 45° C. The mixture was allowed to cool down to 25° C. The residue was purified by Prep-TLC (dichloromethane/methanol=12: 1) to afford 4-{2-[(2S)-2-[2-(1-cyano-1-methylethyl)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (65 mg, 52.9%) as a yellow solid. LC-MS (ESI, m/z) M+1: 1100.

Synthesis of 4-{2-[(2S)-2-[2-(1-cyano-1-methylethyl)phenyl]pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide hydrochloride: Into a 100 mL round-bottom flask were added 4-{2-[(2S)-2-[2-(1-cyano-1-methylethyl)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (65 mg, 0.1 mmol, 1.0 eq), TBAF in THE (1.0 M, 5 mL), ethylenediamine (71 mg, 1.2 mmol, 20.0 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 4-{2-[(2S)-2-[2-(1-cyano-1-methylethyl)phenyl]pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide hydrochloride (11 mg, 17.6%) as a yellow solid. LC-MS (ESI, m/z) M+1: 970. ¹HNMR (400 MHz, DMSO-d₆) δ 12.18 (d, J=75.0 Hz, 1H), 11.56 (s, 1H), 11.13 (d, J=63.5 Hz, 1H), 8.78-8.37 (m, 3H), 8.31 (d, J=2.3 Hz, 1H), 7.79-7.57 (m, 2H), 7.44 (td, J=11.5, 4.5 Hz, 5H), 7.19-6.69 (m, 5H), 6.57 (s, 1H), 6.06-5.93 (m, 1H), 4.90 (d, J=8.6 Hz, 2H), 4.31 (q, J=9.3, 8.6 Hz, 3H), 4.14-3.67 (m, 8H), 3.53 (dd, J=10.6, 7.6 Hz, 1H), 3.47-3.01 (m, 2H), 2.77-2.59 (m, 2H), 2.07 (d, J=40.4 Hz, 3H), 1.84 (d, J=5.2 Hz, 3H), 1.74 (d, J=3.8 Hz, 3H), 1.63 (d, J=12.7 Hz, 2H), 1.55-1.16 (m, 10H).

### Example 20: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N₁,N₂-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of (2S)-2-(2-isopropoxyphenyl)pyrrolidine: Into a 40 mL vial were added (2S)-2-(2-bromophenyl)pyrrolidine (400 mg, 1.7 mmol, 1.0 eq), i-PrOH (5 mL), sodium propan-2-olate (437 mg, 5.3 mmol, 3.0 eq), CuBr (126 mg, 0.9 mmol, 0.5 eq). The resulting mixture was stirred for overnight at 90° C. under nitrogen atmosphere. The mixture was allowed to cool down to 25° C. The resulting mixture was concentrated under vacuum. The crude residue was purified by a flash column (silica gel, dichloromethane/methanol=15: 1) to afford (2S)-2-(2-isopropoxyphenyl)pyrrolidine (106 mg, 29.2%) as a yellow oil. LC-MS (ESI, m/z) M+1: 206. ¹HNMR (300 MHz, DMSO-d₆) δ 7.44 (d, J=7.5 Hz, 1H), 7.18-7.09 (m, 1H), 6.92 (d, J=8.2 Hz, 1H), 6.85 (t, J=7.4 Hz, 1H), 4.61 (p, J=6.0 Hz, 1H), 4.25 (s, 1H), 3.25-2.77 (m, 3H), 2.16 (d, J=11.7 Hz, 1H), 1.69 (q, J=7.4 Hz, 2H), 1.37 (d, J=19.7 Hz, 1H), 1.27 (dd, J=6.0, 1.7 Hz, 6H).

Synthesis of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added (2S)-2-(2-isopropoxyphenyl)pyrrolidine (23 mg, 0.1 mmol, 1.0 eq), N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq), CH₂Cl₂ (5 mL), NaBH(OAc)₃ (47 mg, 0.2 mmol, 2.0 eq). The resulting mixture was stirred for overnight at 25° C. The resulting mixture was concentrated under vacuum. The residue was purified by Prep-TLC (dichloromethane/methanol=13: 1) to afford 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 83.6%) as a yellow solid. LC-MS (ESI, m/z) M+1: 1091.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide: Into a 100 mL round-bottom flask were added 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq), TBAF/THF (1.0 M, 5 mL), ethylenediamine (110 mg, 1.8 mmol, 20.0 eq). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% NH₃·H₂O) and CH₃CN (35% CH₃CN up to 75% in 10 min); Detector, uv. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-y]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide (25 mg, 28.4%) as a yellow solid. LC-MS (ESI, m/z) M+1: 961. ¹HNMR (300 MHz, DMSO-d₆) δ 11.08 (d, J=39.8 Hz, 1H), 8.32 (d, J=15.5 Hz, 2H), 7.68-7.29 (m, 3H), 7.27-6.35 (m, 8H), 6.02 (ddd, J=14.6, 3.5, 1.9 Hz, 1H), 4.67 (s, 2H), 4.38-4.03 (m, 2H), 4.00-3.38 (m, 7H), 3.18 (d, J=52.8 Hz, 9H), 2.22 (d, J=35.9 Hz, 4H), 1.89 (d, J=13.9 Hz, 4H), 1.71-1.38 (m, 8H), 1.29 (s, 9H).

### Example 21: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-[2-(isopropylamino)phenyl]pyrrolidin-1-yl]-7-azaspiro[3.5]nonan-7-yl}-N-{3-nitro-4-[(oxan-4-ylmethyl)amino]benzenesulfonyl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), CH₂Cl₂ (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-{2-[(diphenylmethylidene)amino]phenyl}pyrrolidine-1-carboxylate: Into a 40 mL vial were added tert-butyl (2S)-2-(2-bromophenyl)pyrrolidine-1-carboxylate (2.0 g, 6.1 mmol, 1.0 eq), benzenemethanimine, α-phenyl-(1.7 g, 9.2 mmol, 1.5 eq), toluene (20 mL), t-BuONa (1.2 g, 12.3 mmol, 2.0 eq), Pd(dqpf)Cl₂ (0.9 g, 1.2 mmol, 0.2 eq) and dppf (1.4 g, 2.5 mmol, 0.4 eq). The resulting mixture was stirred for overnight at 140° C. under nitrogen atmosphere. The mixture was allowed to cool down to 25° C. The resulting mixture was diluted with water (300 mL). The resulting mixture was extracted with Ethyl acetate (2×100 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 4) to afford tert-butyl (2S)-2-{2-[(diphenylmethylidene)amino]phenyl}pyrrolidine-1-carboxylate (2 g, 87.9%) as a yellow oil. LC-MS (ESI, m/z) M+1: 427. ¹HNMR (400 MHz, Chloroform-d) δ 7.88-7.78 (m, 2H), 7.45 (dt, J=14.5, 7.0 Hz, 3H), 7.30 (t, J=3.1 Hz, 2H), 7.26-7.06 (m, 4H), 6.98-6.81 (m, 2H), 6.26 (d, J=7.5 Hz, 1H), 5.18 (d, J=35.2 Hz, 1H), 3.63 (t, J=6.8 Hz, 2H), 2.27 (dd, J=22.2, 14.4 Hz, 2H), 1.92-1.80 (m, J=6.1 Hz, 2H), 1.48 (s, 3H), 1.33 (s, 6H).

Synthesis of tert-butyl (2S)-2-(2-aminophenyl)pyrrolidine-1-carboxylate: Into a 100 mL round-bottom flask were added tert-butyl (2S)-2-{2-[(diphenylmethylidene)amino]phenyl}pyrrolidine-1-carboxylate (2.2 g, 5.2 mmol, 1.0 eq), THE (30 mL), 2N citric acid (30 mL). The resulting mixture was stirred for 6 h at 25° C. The resulting mixture was concentrated under vacuum. The residue was basified to pH 8 with saturated NaHCO₃ (aq.). The resulting mixture was extracted with Ethyl acetate (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1: 2) to afford tert-butyl (2S)-2-(2-aminophenyl)pyrrolidine-1-carboxylate (1 g, 77.6%) as a light yellow oil. LC-MS (ESI, m/z) M+1: 263. ¹HNMR (400 MHz, DMSO-d₆) δ 6.90 (td, J=7.6, 1.6 Hz, 1H), 6.72 (d, J=7.6 Hz, 1H), 6.61 (dd, J=8.0, 1.2 Hz, 1H), 6.50 (t, J=7.4 Hz, 1H), 4.88 (bs, 2H), 3.52 (ddd, J=11.2, 7.8, 3.6 Hz, 1H), 3.40 (dt, J=10.9, 7.7 Hz, 1H), 2.22-2.05 (m, 1H), 1.92-1.56 (m, 3H), 1.40 (s, 5H), 1.11 (s, 4H).

Synthesis of tert-butyl (2S)-2-[2-(isopropylamino)phenyl]pyrrolidine-1-carboxylate: Into a 100 mL round-bottom flask were added tert-butyl (2S)-2-(2-aminophenyl)pyrrolidine-1-carboxylate (945 mg, 3.6 mmol, 1.0 eq), CH₂Cl₂ (10 mL), acetone (251 mg, 4.3 mmol, 1.2 eq) and NaBH(OAc)₃ (1.5 g, 7.2 mmol, 2.0 eq). The resulting mixture was stirred for overnight at 25° C. The resulting mixture was concentrated under vacuum. The crude residue was purified by a flash column (silica gel, ethyl acetate/petroleum ether=1:6) to afford tert-butyl (2S)-2-[2-(isopropylamino)phenyl]pyrrolidine-1-carboxylate (823 mg, 75.1%) as a light yellow oil. LC-MS (ESI, m/z) M+1: 305. ¹HNMR (400 MHz, DMSO-d₆) δ 7.02 (ddd, J=8.5, 7.2, 1.7 Hz, 1H), 6.78 (dd, J=7.6, 1.6 Hz, 1H), 6.60 (d, J=8.1 Hz, 1H), 6.53 (t, J=7.4 Hz, 1H), 4.96 (s, 1H), 4.37 (d, J=30.1 Hz, 1H), 3.64 (h, J=6.4 Hz, 1H), 3.57-3.35 (m, 2H), 2.17 (s, 1H), 1.88-1.53 (m, 3H), 1.52-1.10 (m, 15H).

Synthesis of N-isopropyl-2-[(2S)-pyrrolidin-2-yl]aniline hydrochloride: A solution of tert-butyl (2S)-2-[2-(isopropylamino)phenyl]pyrrolidine-1-carboxylate (675 mg, 2.2 mmol, 1.0 eq) in HCl (gas) in 1,4-dioxane (10 mL) was stirred for 1 hour at 25° C. The resulting mixture was concentrated under vacuum. The crude product was re-crystallized from diethyl ether to afford N-isopropyl-2-[(2S)-pyrrolidin-2-yl]aniline hydrochloride (520 mg, 97.4%) as a white solid. LC-MS (ESI, m/z) M+1: 205. ¹HNMR (400 MHz, DMSO-d₆) δ 10.09 (s, 1H), 9.07 (s, 1H), 7.76 (d, J=7.7 Hz, 1H), 7.45 (t, J=7.6 Hz, 1H), 7.26 (s, 2H), 4.74 (s, 1H), 3.75 (tt, J=11.7, 5.7 Hz, 1H), 3.32 (s, 1H), 2.44 (s, 1H), 2.04 (ddd, J=29.1, 17.4, 7.5 Hz, 3H), 1.30 (dd, J=15.7, 6.3 Hz, 6H).

Synthesis of 4-{2-[(2S)-2-[2-(isopropylamino)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-isopropyl-2-[(2S)-pyrrolidin-2-yl]aniline hydrochloride (28 mg, 0.1 mmol, 1.0 eq), N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (104 mg, 0.1 mmol, 1.0 eq), CH₂Cl₂ (3 mL), NaBH(OAc)₃ (50 mg, 0.2 mmol, 2.0 eq). The resulting mixture was stirred for overnight at 25° C. The resulting mixture was concentrated under vacuum. The residue was purified by Prep-TLC (dichloromethane/methanol=13: 1) to afford 4-{2-[(2S)-2-[2-(isopropylamino)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (110 mg, 86.7%) as a yellow solid. LC-MS (ESI, m/z) M+1: 1090.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-[2-(isopropylamino)phenyl]pyrrolidin-1-yl]-7-azaspiro[3.5]nonan-7-yl}-N-{3-nitro-4-[(oxan-4-ylmethyl)amino]benzenesulfonyl}benzamide hydrochloride: Into a 100 mL round-bottom flask were added 4-{2-[(2S)-2-[2-(isopropylamino)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (110 mg, 0.1 mmol, 1.0 eq), TBAF/THF (1.0 M, 5 mL), ethylenediamine (121 mg, 2.0 mmol, 20.0 eq). The resulting mixture was stirred for 3 hours at 70° C. The mixture was allowed to cool down to 25° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-[2-(isopropylamino)phenyl]pyrrolidin-1-yl]-7-azaspiro[3.5]nonan-7-yl}-N-{3-nitro-4-[(oxan-4-ylmethyl)amino]benzenesulfonyl}benzamide hydrochloride (38 mg, 37.8%) as a yellow solid. LC-MS (ESI, m/z) M+1: 960. ¹HNMR (400 MHz, DMSO-d₆) δ 12.31 (s, 1H), 11.12 (d, J=63.5 Hz, 1H), 8.55 (t, J=5.8 Hz, 1H), 8.32 (d, J=2.3 Hz, 1H), 8.15 (s, 1H), 7.66 (dd, J=17.9, 9.2 Hz, 1H), 7.54-7.30 (m, 2H), 7.24-6.99 (m, 3H), 6.98-6.82 (m, 2H), 6.77 (d, J=9.3 Hz, 1H), 6.59 (s, 1H), 6.06-5.96 (m, 1H), 4.59 (q, J=8.5 Hz, 1H), 4.31 (q, J=9.4, 8.6 Hz, 2H), 4.00-3.76 (m, 4H), 3.56 (dd, J=25.2, 16.1 Hz, 3H), 3.45-2.86 (m, 9H), 2.46-2.35 (m, 1H), 2.20 (d, J=48.3 Hz, 6H), 1.64 (d, J=12.7 Hz, 2H), 1.46 (s, 5H), 1.24 (dd, J=21.5, 6.4 Hz, 8H).

### Example 22: Preparation of 4-{2-[(2S)-2-(2-cyclobutylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), DCM (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-(2-cyclobutylphenyl)pyrrolidine-1-carboxylate: Into a 40 mL vial were added tert-butyl (2S)-2-(2-bromophenyl)pyrrolidine-1-carboxylate (600 mg, 1.8 mmol, 1.0 eq) and bromocyclobutane (993 mg, 7.4 mmol, 4.0 eq), DMA (10 mL), Zn (1.2 g, 18.45 mmol, 10.1 eq) Pd(PPh₃)₂Cl₂ (516 mg, 0.7 mmol, 0.4 eq). The resulting mixture was stirred for overnight at 65° C. under nitrogen atmosphere. The mixture was allowed to cool down to 25° C. The resulting mixture was diluted with water (300 mL). The resulting mixture was extracted with Ethyl acetate (2×50 mL). The combined organic layers were washed with brine (1×300 mL), dried over anhydrous Na₂SO₄. After filtration, the filtrate was concentrated under reduced pressure. The crude product was purified by Flash-Prep-HPLC with the following conditions (CH₃CN and 0.05% TFA in water) to afford tert-butyl (2S)-2-(2-cyclobutylphenyl)pyrrolidine-1-carboxylate (130 mg, 23.4%) as a light yellow oil. LC-MS (ESI, m/z) M-t-Bu+CH₃CN: 287.

Synthesis of (2S)-2-(2-cyclobutylphenyl)pyrrolidine: Into a 100 mL round-bottom flask were added tert-butyl (2S)-2-(2-cyclobutylphenyl)pyrrolidine-1-carboxylate (130 mg, 0.4 mmol, 1.0 eq), CH₂Cl₂(8 mL) and TFA (1 mL). The resulting mixture was stirred for overnight at 25° C. The mixture was basified to pH 8 with saturated NaHCO₃ (aq.). The resulting mixture was extracted with CH₂Cl₂ (2×50 mL). The combined organic layers were washed with brine (1×100 mL), dried over anhydrous Na₂SO₄. After filtration, the filtrate was concentrated under reduced pressure. This resulted in (2S)-2-(2-cyclobutylphenyl)pyrrolidine (80 mg, 92.1%) as a yellow oil. LC-MS (ESI, m/z) M+1: 202. ¹HNMR (400 MHz, DMSO-d₆) δ 7.52 (dd, J=7.3, 1.9 Hz, 1H), 7.27 (dd, J=7.3, 1.8 Hz, 1H), 7.15 (pd, J=7.3, 1.7 Hz, 2H), 4.20 (t, J=7.6 Hz, 1H), 3.76 (p, J=8.7 Hz, 1H), 3.04 (ddd, J=9.7, 7.3, 5.2 Hz, 1H), 2.88 (dt, J=9.7, 7.5 Hz, 1H), 2.79-2.62 (m, 1H), 2.26 (ddddd, J=15.1, 12.8, 7.3, 5.6, 2.7 Hz, 2H), 2.19-1.90 (m, 4H), 1.86-1.66 (m, 3H), 1.37-1.27 (m, 1H).

Synthesis of 4-{2-[(2S)-2-(2-cyclobutylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq) and (2S)-2-(2-cyclobutylphenyl)pyrrolidine (23 mg, 0.1 mmol, 1.0 eq), CH₂Cl₂ (3 mL), NaBH(OAc)₃ (47 mg, 0.2 mmol, 2.0 eq). The resulting mixture was stirred for overnight at 25° C. The resulting mixture was concentrated under vacuum. The residue was purified by Prep-TLC (dichloromethane/methanol=13: 1) to afford 4-{2-[(2S)-2-(2-cyclobutylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (61 mg, 50.6%) as a yellow solid. LC-MS (ESI, m/z) M+1: 1087.

Synthesis of 4-{2-[(2S)-2-(2-cyclobutylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide: Into a 100 mL round-bottom flask were added 4-{2-[(2S)-2-(2-cyclobutylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (61 mg, 0.1 mmol, 1.00 eq), 1.0 M TBAF/THF (5 mL), ethylenediamine (68 mg, 1.1 mmol, 20.1 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃ and 0.1% NH₃·H₂O) and CH₃CN (45% CH₃CN up to 85% in 10 min); Detector, UV. This resulted in 4-{2-[(2S)-2-(2-cyclobutylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide (15 mg, 27.9%) as a yellow solid. LC-MS (ESI, m/z) M+1: 957. ¹HNMR (400 MHz, DMSO-d₆) δ 11.10 (d, J=49.3 Hz, 1H), 8.57-8.23 (m, 2H), 7.55 (s, 2H), 7.43-7.00 (m, 5H), 6.69 (dd, J=58.1, 18.7 Hz, 4H), 6.06-5.97 (m, 1H), 4.73-4.02 (m, 3H), 3.97-3.65 (m, 5H), 3.49 (dt, J=38.4, 9.0 Hz, 3H), 3.30-2.71 (m, 10H), 2.43-1.56 (m, 14H), 1.54-1.16 (m, 10H).

### Example 23: Preparation of 4-{2-[(2S)-2-(2-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10 g, 41.786 mmol, 1.00 eq), ethyl acetate (20 mL) and 2.0 M HCl in ethyl acetate (80 mL) at 25° C. The resulting mixture was stirred for 4 h at 25° C. The resulting mixture was concentrated under reduced pressure. This resulted in 7-azaspiro[3.5]nonan-2-one hydrochloride (7.3 g, 99.45%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.56 mmol, 1.00 eq), DMF (100 mL), Cs₂CO₃ (33.85 g, 103.89 mmol, 2.5 eq) and methyl 2-bromo-4-fluorobenzoate (10.5 g, 45.05 mmol, 1.0 eq) at 25° C. The resulting mixture was stirred for 8 h at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The residue was purified by silica gel column chromatography, eluted with petroleum ether/ethyl acetate (10:01-2:1) to afford methyl 2-bromo-4-{2-oxo-7-azaspiro[3.5]nonan-7-yl}benzoate (6.8 g, 46.46%) as light yellow solid. LC-MS (ESI, m/z) M+1: 352. ¹H NMR (300 MHz, DMSO-d) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1 g, 2.839 mmol, 1.00 eq) and MeOH (5 mL), water (5 mL), NaOH (0.45 g, 11.356 mmol, 4 eq) at 25° C. The resulting mixture was stirred for 2 h at 40° C. The mixture was allowed to cool down to 25° C. The resulting mixture was diluted with water (20 mL). The mixture/residue was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.90%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97 1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1 g, 2.957 mmol, 1.00 eq) and 3-nitro-4-[(oxan-4-ylmethyl)amino]benzenesulfonamide (1.03 g, 3.253 mmol, 1.1 eq), DMAP (0.72 g, 5.914 mmol, 2.0 eq), DCM (10 mL), EDCI (0.68 g, 3.548 mmol, 1.2 eq) at 25° C. The resulting mixture was stirred for 3 h at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with petroleum ether/ethyl acetate (5:1-0:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.93%) as yellow solid. LC-MS (ESI, m/z) M+1: 635/337. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90 3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77 1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.944 mmol, 1.00 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328.06 mg, 0.944 mmol, 1.0 eq), Cs₂CO₃ (617.11 mg, 1.888 mmol, 2 eq), copper(I) iodide (35.96 mg, 0.189 mmol, 0.2 eq), N₁,N₂-diphenyloxalamide (45.37 mg, 0.189 mmol, 0.2 eq), dimethylformamide (6 mL) at room temperature. The resulting mixture was stirred for 2 h at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The residue was purified by silica gel column chromatography, eluted with (Ethyl acetate/DCM=1:1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 46.97%) as yellow solid. LC-MS (ESI, m/z) M+1: 902; ¹HNMR (300 MHz, DMSO-d) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of (S)—N-[(2-chlorophenyl)methylidene]-2-methylpropane-2-sulfinamide: Into a 500 mL round-bottom flask were added 2-chlorobenzaldehyde (15 g, 106.708 mmol, 1.00 eq), THE (200 mL), (S)-2-methylpropane-2-sulfinamide (13 g, 107.261 mmol, 1.01 eq), Ti(OEt)₄ (48.9 g, 214.371 mmol, 2.01 eq). The resulting mixture was stirred for 3 h at 55° C. The mixture was allowed to cool down to 25° C. The resulting mixture was concentrated under vacuum. The resulting solution was diluted with 1000/1000 mL of water and Ethyl acetate. The solids were filtered out. The organic layer was separated from the filtration. The mixture was dried over anhydrous sodium sulfate and concentrated under vacuum. This resulted in (S)—N-[(2-chlorophenyl)methylidene]-2-methylpropane-2-sulfinamide (23.7 g, 91.12%) as a yellow oil. LC-MS (ESI, m/z) M+1: 244; ¹HNMR (400 MHz, Chloroform-d) δ 9.03 (d, J=0.7 Hz, 1H), 8.09-8.00 (m, 1H), 7.48-7.35 (m, 2H), 7.33 (dddd, J=8.5, 6.0, 2.8, 0.7 Hz, 1H), 1.27 (s, 9H).

Synthesis of (S)—N-[(1S)-1-(2-chlorophenyl)-3-(1,3-dioxan-2-yl)propyl]-2-methylpropane-2-sulfinamide: Into a 500-mL 3-necked round-bottom flask purged and maintained with an inert atmosphere of nitrogen, were placed Mg (4.36 g, 179.500 mmol, 4 eq), 12 (1.14 g, 4.487 mmol, 0.1 eq), THE (200.00 mL), the resulting solution was warmed to 65° C., followed by the addition of 2-(2-bromoethyl)-1,3-dioxane (3.48 g, 17.95 mmol, 0.4 eq) in THE (50 mL) at 65 degrees C. After addition completed, the mixture was cooled to room temperature, another 2-(2-bromoethyl)-1,3-dioxane (31.34 g, 161.55 mmol, 3.6 eq) in THE (150 mL) was added. To the above solution was added a solution of (S)—N-[(2-fluorophenyl)methylidene]-2-methylpropane-2-sulfinamide (10.2 g, 44.875 mmol, 1.00 eq) in DCM (50 mL) dropwise at −40° C. The resulting solution was stirred for 1 hours at −40° C. in a liquid nitrogen bath. The reaction was then quenched by the addition of 1000 mL of aqueous NH₄Cl. The resulting solution was extracted with 3×300 mL of dichloromethane and the organic layers combined. The resulting mixture was washed with 1×1000 mL of brine. The mixture was dried over anhydrous sodium sulfate and concentrated under vacuum. This resulted in (S)—N-[(1S)-1-(2-chlorophenyl)-3-(1,3-dioxan-2-yl)propyl]-2-methylpropane-2-sulfinamide (15 g, 92.35%) as a yellow oil. LC-MS (ESI, m/z) M+1: 360

Synthesis of (2S)-2-(2-chlorophenyl)pyrrolidine: Into a 500-mL round-bottom flask, were placed (S)—N-[(1S)-1-(2-chlorophenyl)-3-(1,3-dioxan-2-yl)propyl]-2-methylpropane-2-sulfinamide (15 g, 41.677 mmol, 1.00 eq), TFA (100.00 mL), water (25 mL), the resulting solution was stirred for 1 h at 25° C. TFA (100.00 mL), Et₃SiH (14.54 g, 125.031 mmol, 3 eq) was added. The resulting solution was stirred overnight at room temperature. The resulting mixture was concentrated. The resulting solution was diluted with 500 mL of water. The resulting solution was extracted with 2×200 mL of MTBE and the organic layers combined. The resulting mixture was washed with 1×500 mL of HCl (2 mol/L). The pH value of the aqueous was adjusted to 13 with NaOH (4 mol/L). The resulting solution was extracted with 3×300 mL of dichloromethane and the organic layers combined and dried over anhydrous sodium sulfate and concentrated. This resulted in (2S)-2-(2-chlorophenyl)pyrrolidine (2.3 g, 30.38%) as light yellow oil. LC-MS (ESI, m/z) M+1: 182; ¹HNMR (400 MHz, DMSO-d₆) δ 7.68 (dd, J=7.8, 1.8 Hz, 1H), 7.36 (dd, J=7.8, 1.4 Hz, 1H), 7.30 (td, J=7.5, 1.3 Hz, 1H), 7.21 (td, J=7.6, 1.9 Hz, 1H), 4.38 (t, J=7.4 Hz, 1H), 3.02 (dt, J=9.7, 6.3 Hz, 1H), 2.94 (dt, J=9.6, 7.3 Hz, 1H), 2.75 (s, 1H), 2.38-2.16 (m, 1H), 1.80-1.65 (m, 2H), 1.33 (dq, J=12.2, 7.4 Hz, 1H).

Synthesis of 4-{2-[(2S)-2-(2-chlorophenyl)pyrrolidin-1-yl]-7-azaspiro[3.5]nonan-7-yl}-N-{3-nitro. 4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.111 mmol, 1.00 eq), (2S)-2-(2-chlorophenyl)pyrrolidine (20 mg, 0.110 mmol, 0.99 eq), DCM (3 mL), NaBH(OAc)₃ (47 mg, 0.222 mmol, 2.00 eq). The resulting mixture was stirred for overnight at 25° C. The resulting mixture was concentrated under vacuum. The residue was purified by Prep-TLC (CH₂Cl₂/MeOH 12:1) to afford 4-{2-[(2S)-2-(2-chlorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 84.49%) as a yellow solid. LC-MS (ESI, m/z) M+1: 1067

Synthesis of 4-{2-[(2S)-2-(2-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]. N-{3-nitro-4-[(oxan-4-ylmethyl)amino]benzenesulfonyl}benzamide hydrochloride: Into a 100 mL round-bottom flask were added 4-{2-[(2S)-2-(2-chlorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.094 mmol, 1.00 eq), 1.0 M TBAF in THE (5 mL), ethylenediamine (113 mg, 1.880 mmol, 20.08 eq). The resulting mixture was stirred for 3 h at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, Water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 4-{2-[(2S)-2-(2-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide hydrochloride (10.8 mg, 11.84%) as a yellow solid. LC-MS (ESI, m/z) M+1: 937. ¹HNMR (400 MHz, DMSO-d₆) δ 12.32 (s, 1H), 11.69 (s, 1H), 11.13 (d, J=64.3 Hz, 1H), 8.79-8.15 (m, 3H), 7.72-6.53 (m, 11H), 6.00 (s, 1H), 4.81 (d, J=6.1 Hz, 1H), 4.64-4.53 (m, 1H), 4.31 (d, J=7.7 Hz, 2H), 3.87 (d, J=11.6 Hz, 4H), 3.70 (s, 2H), 3.54 (d, J=9.9 Hz, 1H), 3.46-2.93 (m, 11H), 2.28 (s, 2H), 2.13 (s, 5H), 1.90 (s, 2H), 1.73-1.14 (m, 12H).

Synthesis of (S)—N-[(2-chlorophenyl)methylidene]-2-methylpropane-2-sulfinamide: Into a 500 mL round-bottom flask were added 2-chlorobenzaldehyde (15.0 g, 106.7 mmol, 1.0 eq), THE (200 mL), (S)-2-methylpropane-2-sulfinamide (13.0 g, 107.2 mmol, 1.0 eq), Ti(OEt)₄ (48.9 g, 214.4 mmol, 2.0 eq). The resulting mixture was stirred for 3 hours at 55° C. The mixture was allowed to cool down to 25° C. The resulting mixture was concentrated under vacuum. The resulting solution was diluted with water and Ethyl acetate (1000/1000 mL). The solids were filtered out. The organic layer was separated from the filtration. The mixture was dried over anhydrous sodium sulfate and concentrated under vacuum. This resulted in (S)—N-[(2-chlorophenyl)methylidene]-2-methylpropane-2-sulfinamide (24 g, 91.1%) as a yellow oil. LC-MS (ESI, m/z) M+1: 244. ¹HNMR (400 MHz, Chloroform-d) δ 9.03 (d, J=0.7 Hz, 1H), 8.09-8.00 (m, 1H), 7.48-7.35 (m, 2H), 7.33 (dddd, J=8.5, 6.0, 2.8, 0.7 Hz, 1H), 1.27 (s, 9H).

Synthesis of (S)—N-[(1S)-1-(2-chlorophenyl)-3-(1,3-dioxan-2-yl)propyl]-2-methylpropane-2-sulfinamide: Into a 500-mL 3-necked round-bottom flask purged and maintained with an inert atmosphere of nitrogen, were placed Mg (4.4 g, 179.5 mmol, 4.0 eq), I₂ (1.1 g, 4.5 mmol, 0.1 eq), THE (200 mL), the resulting solution was warmed to 65° C., followed by the addition of 2-(2-bromoethyl)-1,3-dioxane (3.5 g, 17.9 mmol, 0.4 eq) in THE (50 mL) at 65° C. After addition completed, the mixture was cooled to 25° C., another 2-(2-bromoethyl)-1,3-dioxane (31.3 g, 161.5 mmol, 3.6 eq) in THE (150 mL) was added. To the above solution was added a solution of (S)—N-[(2-fluorophenyl)methylidene]-2-methylpropane-2-sulfinamide (10.2 g, 44.8 mmol, 1.0 eq) in CH₂Cl₂ (50 mL) dropwise at −40° C. The resulting solution was stirred for 1 hour at −40° C. in a liquid nitrogen bath. The reaction was then quenched by the addition of aqueous NH₄Cl (1000 mL). The resulting solution was extracted with of dichloromethane (3×300 mL) and the organic layers combined. The resulting mixture was washed with brine (1×1000 mL), dried over anhydrous sodium sulfate. After filtration, the filtrate was concentrated under reduced pressure. This resulted in (S)—N-[(1S)-1-(2-chlorophenyl)-3-(1,3-dioxan-2-yl)propyl]-2-methylpropane-2-sulfinamide (15 g, 92.3%) as a yellow oil. LC-MS (ESI, m/z) M+1: 360.

Synthesis of (2S)-2-(2-chlorophenyl)pyrrolidine: Into a 500-mL round-bottom flask, were placed (S)—N-[(1S)-1-(2-chlorophenyl)-3-(1,3-dioxan-2-yl)propyl]-2-methylpropane-2-sulfinamide (15.0 g, 41.7 mmol, 1.0 eq), TFA (100 mL), water (25 mL), the resulting solution was stirred for 1 hour at 25° C. TFA (100 mL), Et₃SiH (14.5 g, 125.0 mmol, 3.0 eq) was added. The resulting solution was stirred overnight at 25° C. The resulting mixture was concentrated. The resulting solution was diluted with water (500 mL). The resulting solution was extracted with MTBE (2×200 mL) and the organic layers combined. The resulting mixture was washed with 2.0 M HCl (1×500 mL). The pH value of the aqueous was adjusted to 13 with NaOH (4 M). The resulting solution was extracted with dichloromethane (3×300 mL) and the organic layers combined and dried over anhydrous Na₂SO₄. After filtration, the filtrate was concentrated under reduced pressure. This resulted in (2S)-2-(2-chlorophenyl)pyrrolidine (2 g, 30.4%) as light yellow oil. LC-MS (ESI, m/z) M+1: 182. ¹HNMR (400 MHz, DMSO-d₆) δ 7.68 (dd, J=7.8, 1.8 Hz, 1H), 7.36 (dd, J=7.8, 1.4 Hz, 1H), 7.30 (td, J=7.5, 1.3 Hz, 1H), 7.21 (td, J=7.6, 1.9 Hz, 1H), 4.38 (t, J=7.4 Hz, 1H), 3.02 (dt, J=9.7, 6.3 Hz, 1H), 2.94 (dt, J=9.6, 7.3 Hz, 1H), 2.75 (s, 1H), 2.38-2.16 (m, 1H), 1.80-1.65 (m, 2H), 1.33 (dq, J=12.2, 7.4 Hz, 1H).

Synthesis of 4-{2-[(2S)-2-(2-chlorophenyl)pyrrolidin-1-yl]-7-azaspiro[3.5]nonan-7-yl}-N-{3-nitro. 4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq), (2S)-2-(2-chlorophenyl)pyrrolidine (20 mg, 0.1 mmol, 1.0 eq), CH₂Cl₂ (3 mL), NaBH(OAc)₃ (47 mg, 0.2 mmol, 2.0 eq). The resulting mixture was stirred for overnight at 25° C. The resulting mixture was concentrated under vacuum. The residue was purified by Prep-TLC (dichloromethane/methanol=12: 1) to afford 4-{2-[(2S)-2-(2-chlorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 84.5%) as a yellow solid. LC-MS (ESI, m/z) M+1: 1067.

Synthesis of 4-{2-[(2S)-2-(2-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide hydrochloride: Into a 100 mL round-bottom flask were added 4-{2-[(2S)-2-(2-chlorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq), TBAF in THE (1.0 M, 5 mL), ethylenediamine (113 mg, 1.9 mmol, 20.0 eq). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 4-{2-[(2S)-2-(2-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide hydrochloride (11 mg, 11.8%) as a yellow solid. LC-MS (ESI, m/z) M+1: 937. ¹HNMR (400 MHz, DMSO-d₆) δ 12.32 (s, 1H), 11.69 (s, 1H), 11.13 (d, J=64.3 Hz, 1H), 8.79-8.15 (m, 3H), 7.72-6.53 (m, 10H), 6.00 (s, 1H), 4.81 (d, J=6.1 Hz, 1H), 4.64-4.53 (m, 1H), 4.31 (d, J=7.7 Hz, 2H), 3.87 (d, J=11.6 Hz, 4H), 3.70 (s, 2H), 3.54 (d, J=9.9 Hz, 1H), 3.46-2.93 (m, 11H), 2.28 (s, 2H), 2.13 (s, 3H), 1.90 (s, 2H), 1.73-1.14 (m, 8H).

### Example 24: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-fluorophenyl)pyrrolidin-1-yl]-7-azaspiro[3.5]nonan-7-yl}-N-{3-nitro-4-[(oxan-4-ylmethyl)amino]benzenesulfonyl}benzamide hydrochloride

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N₁,N₂-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of (S)—N-[(2-fluorophenyl)methylidene]-2-methylpropane-2-sulfinamide: Into a 500 mL round-bottom flask were added 2-fluor-benzaldehyd (15.0 g, 120.8 mmol, 1.0 eq), THE (200 mL), (S)-2-methylpropane-2-sulfinamide (14.6 g, 120.4 mmol, 1.0 eq), Ti(OEt)₄ (55.2 g, 241.9 mmol, 2.0 eq). The resulting mixture was stirred for 3 hours at 55° C. The mixture was allowed to cool down to 25° C. The resulting mixture was concentrated under vacuum. The resulting solution was diluted with water and Ethyl acetate (1000/1000 mL). The solids were filtered out. The organic layer was separated from the filtration. The mixture was dried over anhydrous sodium sulfate. After filtration, the filtrate was concentrated under reduced pressure. This resulted in (S)—N-[(2-fluorophenyl)methylidene]-2-methylpropane-2-sulfinamide (26 g, 96.4%) as a yellow oil. LC-MS (ESI, m/z) M+1: 228. ¹HNMR (400 MHz, Chloroform-d) δ 8.91 (s, 1H), 8.00 (td, J=7.5, 1.8 Hz, 1H), 7.50 (dddd, J=8.3, 7.2, 5.3, 1.8 Hz, 1H), 7.29-7.09 (m, 2H), 1.27 (s, 9H).

Synthesis of (S)—N-[(1S)-3-(1,3-dioxan-2-yl)-1-(2-fluorophenyl)propyl]-2-methylpropane-2-sulfinamide: Into a 500-mL 3-necked round-bottom flask purged and maintained with an inert atmosphere of nitrogen, were placed Mg (4.4 g, 179.5 mmol, 4.0 eq), I₂ (1.1 g, 4.4 mmol, 0.1 eq), THE (200 mL), the resulting solution was warmed to 65° C., followed by the addition of 2-(2-bromoethyl)-1,3-dioxane (3.5 g, 17.9 mmol, 0.4 eq) in THE (50 mL) at 65° C. After addition completed, the mixture was cooled to 25° C., another 2-(2-bromoethyl)-1,3-dioxane (31.3 g, 161.5 mmol, 3.6 eq) in THE (150 mL) was added. To the above solution was added a solution of (S)—N-[(2-fluorophenyl)methylidene]-2-methylpropane-2-sulfinamide (10.2 g, 44.8 mmol, 1.0 eq) in CH₂Cl₂ (50 mL) dropwise at −40° C. The resulting solution was stirred for 1 hour at −40° C. in a liquid nitrogen bath. The reaction was then quenched by the addition of aqueous NH₄Cl (1000 mL). The resulting solution was extracted with of dichloromethane (3×300 mL) and the organic layers combined. The resulting mixture was washed with brine (1×1000 mL). The mixture was dried over anhydrous sodium sulfate. After filtration, the filtrate was concentrated under reduced pressure. This resulted in (S)—N-[(1S)-3-(1,3-dioxan-2-yl)-1-(2-fluorophenyl)propyl]-2-methylpropane-2-sulfinamide (15 g, 97.3%) as a yellow oil. LC-MS (ESI, m/z) M+1: 344.

Synthesis of (2S)-2-(2-fluorophenyl)pyrrolidine: Into a 500-mL round-bottom flask, were placed (S)—N-[(1S)-3-(1,3-dioxan-2-yl)-1-(2-fluorophenyl)propyl]-2-methylpropane-2-sulfinamide (15.0 g, 43.6 mmol, 1.0 eq), TFA (100 mL), water (25 mL), the resulting solution was stirred for 1 hour at 25° C. TFA (100 mL), Et₃SiH (15.2 g, 131.0 mmol, 3.0 eq) was added. The resulting solution was stirred overnight at 25° C. The resulting mixture was concentrated. The resulting solution was diluted with water (500 mL). The resulting solution was extracted with MTBE (2×200 mL) and the organic layers combined. The resulting mixture was washed with 2.0 M HCl (1×500 mL). The pH value of the aqueous was adjusted to 13 with NaOH (4 M). The resulting solution was extracted with dichloromethane (3×300 mL) and the organic layers combined and dried over anhydrous sodium sulfate. After filtration, the filtrate was concentrated under reduced pressure. This resulted in (2S)-2-(2-fluorophenyl)pyrrolidine (1 g, 14.3%) as light yellow oil. LC-MS (ESI, m/z) M+1: 166. ¹HNMR (400 MHz, DMSO-d₆) δ 7.57 (td, J=7.6, 1.9 Hz, 1H), 7.24 (tdd, J=7.5, 5.3, 1.9 Hz, 1H), 7.18-7.10 (m, 1H), 7.15-7.03 (m, 1H), 4.29 (t, J=7.5 Hz, 1H), 3.06-2.83 (m, 2H), 2.24-2.06 (m, 1H), 1.81-1.64 (m, 2H), 1.52-1.34 (m, 1H).

Synthesis of 4-{2-[(2S)-2-(2-fluorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq), (2S)-2-(2-fluorophenyl)pyrrolidine (18 mg, 0.1 mmol, 1.0 eq), CH₂Cl₂ (3 mL), NaBH(OAc)₃ (47 mg, 0.2 mmol, 2.0 eq). The resulting mixture was stirred for overnight at 25° C. The resulting mixture was concentrated under vacuum. The residue was purified by Prep-TLC (dichloromethane/methanol=12: 1) to afford 4-{2-[(2S)-2-(2-fluorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (97 mg, 83.2%) as a yellow solid. LC-MS (ESI, m/z) M+1: 1051.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-fluorophenyl)pyrrolidin-1-yl]-7-azaspiro[3.5]nonan-7-yl}-N-{3-nitro-4-[(oxan-4-ylmethyl)amino]benzenesulfonyl}benzamide hydrochloride: Into a 100 mL round-bottom flask were added 4-{2-[(2S)-2-(2-fluorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (97 mg, 0.1 mmol, 1.0 eq), TBAF in THE (1.0 M, 5 mL), ethylenediamine (111 mg, 1.8 mmol, 20.0 eq). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-fluorophenyl)pyrrolidin-1-yl]-7-azaspiro[3.5]nonan-7-yl}-N-{3-nitro-4-[(oxan-4-ylmethyl)amino]benzenesulfonyl}benzamide hydrochloride (32 mg, 36.2%) as a yellow solid. LC-MS (ESI, m/z) M+1: 921. ¹HNMR (400 MHz, DMSO-d₆) δ 12.32 (s, 1H), 11.59-10.98 (m, 2H), 8.73-8.41 (m, 1H), 8.32 (d, J=2.3 Hz, 1H), 8.06 (d, J=7.8 Hz, 1H), 7.74-7.58 (m, 1H), 7.53-7.38 (m, 2H), 7.36-7.21 (m, 2H), 7.17-7.02 (m, 1H), 6.99-6.84 (m, 2H), 6.77 (d, J=9.4 Hz, 1H), 6.59 (s, 1H), 6.07-5.95 (m, 1H), 4.69-4.58 (m, 2H), 4.31 (q, J=8.6, 8.1 Hz, 2H), 4.00-3.78 (m, 5H), 3.71-3.50 (m, 2H), 3.47-2.99 (m, 10H), 2.42 (d, J=7.3 Hz, 1H), 2.21 (d, J=37.5 Hz, 5H), 1.90 (s, 1H), 1.78-1.37 (m, 8H), 1.27 (d, J=13.5 Hz, 2H).

### Example 25: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methanesulfonamidophenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of 4-{2-[(2S)-2-(2-bromophenyl)pyrrolidin-1-yl]-7-azaspiro[3.5]nonan-7-yl}-N-{3-nitro. 4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 100 mL round-bottom flask were added (2S)-2-(2-bromophenyl)pyrrolidine (250 mg, 1.1 mmol, 1.00 eq) and N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (1.0 g, 1.1 mmol, 1.0 eq), CH₂Cl₂ (20 mL), NaBH(OAc)₃ (471 mg, 2.2 mmol, 2.0 eq). The resulting mixture was stirred for overnight at 25° C. The resulting mixture was concentrated under vacuum. The crude residue was purified by a flash column (silica gel, dichloromethane/ethyl acetate (including 10% MeOH) (3:1) to afford 4-{2-[(2S)-2-(2-bromophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (669 mg, 54.3%) as a yellow solid. LC-MS (ESI, m/z) M+1: 1111.

Synthesis of 4-{2-[(2S)-2-(2-methanesulfonamidophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 4-{2-[(2S)-2-(2-bromophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (290 mg, 0.3 mmol, 1.0 eq), DMF (5 mL), methanesulfonamide (75 mg, 0.8 mmol, 3.0 eq), proline (12 mg, 0.1 mmol, 0.4 eq), CuI (40 mg, 0.2 mmol, 0.8 eq), K₂CO₃ (108 mg, 0.8 mmol, 3.0 eq). The resulting mixture was stirred for overnight at 85° C. under nitrogen atmosphere. The mixture was allowed to cool down to 25° C. The resulting mixture was diluted with water (100 mL). The resulting mixture was extracted with CH₂Cl₂ (3×50 mL). The combined organic layers were washed with brine (1×200 mL), dried over anhydrous Na₂SO₄. After filtration, the filtrate was concentrated under reduced pressure. The crude residue was purified by a flash column (silica gel, dichloromethane/ethyl acetate (including 10% MeOH) (4: 1) to afford 4-{2-[(2S)-2-(2-methanesulfonamidophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (90 mg, 30.6%) as a yellow solid. LC-MS (ESI, m/z) M+1: 1126.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methanesulfonamidophenyl)pyrrolidin-1-yl]-7-azaspiro[3.5]nonan-7-yl}-N-{3-nitro-4-[(oxan-4-ylmethyl)amino]benzenesulfonyl}benzamide: Into a 100 mL round-bottom flask were added 4-{2-[(2S)-2-(2-methanesulfonamidophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (90 mg, 0.1 mmol, 1.0 eq), TBAF in THE (1.0 M, 5 mL), ethylenediamine (96 mg, 1.6 mmol, 20.0 eq). The resulting mixture was stirred for 5 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, Water (10 mmol/L NH₄HCO₃ and 0.1% NH₃·H₂O) and CH₃CN (45% CH₃CN up to 85% in 10 min); Detector, UV. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methanesulfonamidophenyl)pyrrolidin-1-yl]-7-azaspiro[3.5]nonan-7-yl}-N-{3-nitro-4-[(oxan-4-ylmethyl)amino]benzenesulfonyl}benzamide (23 mg, 28.9%) as a yellow solid. LC-MS (ESI, m/z) M+1: 996. ¹HNMR (400 MHz, DMSO-d₆) δ 12.31 (s, 1H), 11.57 (s, 1H), 11.12 (d, J=63.4 Hz, 1H), 8.55 (s, 1H), 8.31 (d, J=2.2 Hz, 1H), 7.51 (d, J=54.3 Hz, 2H), 7.33 (d, J=8.0 Hz, 1H), 7.26-7.15 (m, 2H), 7.09 (d, J=14.0 Hz, 1H), 7.00 (d, J=8.1 Hz, 1H), 6.84 (d, J=17.6 Hz, 3H), 6.58 (s, 1H), 6.06-5.98 (m, 1H), 4.61 (s, 1H), 4.32 (s, 2H), 3.87 (d, J=11.6 Hz, 4H), 3.66-3.46 (m, 2H), 3.28 (d, J=10.8 Hz, 5H), 3.13 (s, 8H), 2.36 (d, J=8.8 Hz, 1H), 2.13 (d, J=28.5 Hz, 1H), 1.85 (s, 4H), 1.77-1.55 (m, 5H), 1.46 (d, J=19.2 Hz, 5H), 1.27 (d, J=12.9 Hz, 2H).

### Example 26: Preparation of 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide

Synthesis of tert-butyl 2-[(2S)-2-(2-cyanophenyl)pyrrolidin-1-yl]-7-azaspiro[3.5]nonane-7-carboxylate: Into a 40 mL vial were added tert-butyl 2-[(2S)-2-(2-bromophenyl)pyrrolidin-1-yl]-7-azaspiro[3.5]nonane-7-carboxylate (1.0 g, 2.2 mmol, 1.0 eq), Zn(CN)₂ (792 mg, 6.7 mmol, 3.0 eq), Zn (292 mg, 4.4 mmol, 2.0 eq), DMA (10 mL), dppf (253 mg, 0.5 mmol, 0.2 eq), Pd₂(dba)₃ (209 mg, 0.2 mmol, 0.1 eq). The resulting mixture was stirred for overnight at 100° C. under nitrogen atmosphere. The mixture was allowed to cool down to 25° C. The resulting mixture was diluted with water (300 mL). The resulting mixture was extracted with Ethyl acetate (2×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=2:3) to afford tert-butyl 2-[(2S)-2-(2-cyanophenyl)pyrrolidin-1-yl]-7-azaspiro[3.5]nonane-7-carboxylate (820 mg, 93.2%) as a yellow oil. LC-MS (ESI, m/z) M+1: 396.

Synthesis of 2-[(2S)-1-{7-azaspiro[3.5]nonan-2-yl}pyrrolidin-2-yl]benzonitrile hydrochloride: A solution of tert-butyl 2-[(2S)-2-(2-cyanophenyl)pyrrolidin-1-yl]-7-azaspiro[3.5]nonane-7-carboxylate (820 mg, 2.1 mmol, 1.0 eq) in HCl (gas) in 1,4-dioxane (10 mL) was stirred for 1 hour at 25° C. The resulting mixture was concentrated under vacuum. This resulted in 2-[(2S)-1-{7-azaspiro[3.5]nonan-2-yl}pyrrolidin-2-yl]benzonitrile hydrochloride (700 mg, crude) as a light yellow solid. LC-MS (ESI, m/z) M+1: 296.

Synthesis of methyl 2-bromo-4-{2-[(2S)-2-(2-cyanophenyl)pyrrolidin-1-yl]-7-azaspiro[3.5]nonan. 7-yl}benzoate: Into a 40 mL round-bottom flask were added 2-[(2S)-1-{7-azaspiro[3.5]nonan-2-yl}pyrrolidin-2-yl]benzonitrile hydrochloride (700 mg, 2.1 mmol, 1.0 eq), DMSO (10 mL), methyl 2-bromo-4-fluorobenzoate (492 mg, 2.1 mmol, 1.0 eq), Na₂CO₃ (447 mg, 4.2 mmol, 2.0 eq). The resulting mixture was stirred for overnight at 110° C. The mixture was allowed to cool down to 25° C. The resulting mixture was diluted with water (300 mL). The resulting mixture was extracted with Ethyl acetate (2×100 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1:1) to afford methyl 2-bromo-4-{2-[(2S)-2-(2-cyanophenyl)pyrrolidin-1-yl]-7-azaspiro[3.5]nonan-7-yl}benzoate (670 mg, 62.4%) as a yellow oil. LC-MS (ESI, m/z) M+1: 508. ¹HNMR (400 MHz, Chloroform-d) δ 7.78 (dd, J=18.3, 8.4 Hz, 2H), 7.59 (qd, J=7.7, 1.3 Hz, 2H), 7.33 (td, J=7.6, 1.2 Hz, 1H), 7.06 (d, J=2.5 Hz, 1H), 6.74 (dd, J=9.0, 2.6 Hz, 1H), 3.87 (s, 3H), 3.83 (t, J=7.9 Hz, 1H), 3.29-3.05 (m, 6H), 2.55-2.29 (m, 2H), 1.97 (ddt, J=9.9, 7.2, 3.5 Hz, 2H), 1.91-1.47 (m, 9H), 1.39-1.25 (m, 2H).

Synthesis of methyl 4-{2-[(2S)-2-(2-cyanophenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate: Into a 40 mL vial were added methyl 2-bromo-4-{2-[(2S)-2-(2-cyanophenyl)pyrrolidin-1-yl]-7-azaspiro[3.5]nonan-7-yl}benzoate (640 mg, 1.3 mmol, 1.3 eq), (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (347 mg, 1.0 mmol, 1.0 eq), toluene (10 mL), Cs₂CO₃ (975 mg, 3.0 mmol, 3.0 eq), Xantphos (115 mg, 0.2 mmol, 0.2 eq), Pd₂(dba)₃·CHCl₃ (103 mg, 0.1 mmol, 0.1 eq). The resulting mixture was stirred for 2 hours at 100° C. under nitrogen atmosphere. The mixture was allowed to cool down to 25° C. The resulting mixture was concentrated under vacuum. The crude residue was purified by a flash column (silica gel, ethyl acetate/petroleum ether=3: 2) to afford methyl 4-{2-[(2S)-2-(2-cyanophenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (580 mg, 74.9%) as a yellow solid. LC-MS (ESI, m/z) M+1: 775.

Synthesis of methyl 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoate: Into a 100 mL round-bottom flask were added methyl 4-{2-[(2S)-2-(2-cyanophenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (550 mg, 0.7 mmol, 1.0 eq), TBAF in THE (1.0 M, 10 mL), ethylenediamine (640 mg, 10.6 mmol, 15.0 eq). The resulting mixture was stirred for 6 hours at 70° C. The mixture was allowed to cool down to 25° C. The resulting mixture was diluted with water (200 mL). The resulting mixture was extracted with Ethyl acetate (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate) to afford methyl 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoate (237 mg, 51.8%) as a light yellow solid. LC-MS (ESI, m/z) M+1: 645.

Synthesis of 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoic acid: Into a 100 mL round-bottom flask were added methyl 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoate (230 mg, 0.4 mmol, 1.0 eq), MeOH (5 mL), dioxane (5 mL), 4 M NaOH (1 mL). The resulting mixture was stirred for overnight at 40° C. The mixture was allowed to cool down to 25° C. The resulting mixture was concentrated under vacuum. The mixture was acidified to pH 5 with 2.0 M HCl. The resulting mixture was extracted with dichloromethane (2×50 mL). The combined organic layers were washed with brine (1×100 mL), dried over anhydrous Na₂SO₄. After filtration, the filtrate was concentrated under reduced pressure. The crude residue was purified by a flash column (silica gel, dichloromethane/methanol=10: 1) to afford 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoic acid (100 mg, 44.4%) as a yellow solid. LC-MS (ESI, m/z) M+1: 631.

Synthesis of 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide: Into a 20 mL vial were added 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoic acid (34 mg, 0.1 mmol, 1.0 eq), 3-nitro-4-[(oxan-4-ylmethyl)amino]benzenesulfonamide (17 mg, 0.1 mmol, 1.0 eq), CH₂Cl₂ (3 mL), EDCI (21 mg, 0.1 mmol, 2.0 eq), DMAP (26 mg, 0.2 mmol, 4.0 eq). The resulting mixture was stirred for overnight at 30° C. The resulting mixture was concentrated under vacuum. The residue was purified by Prep-TLC (dichloromethane/methanol=12: 1), then purified by Prep-HPLC with the following conditions (Prep-HPLC-006): Column, YMC-Actus Triart C18 ExRS, 30*150 mm, 5 μm; mobile phase, water (10 mmol/L NH₄HCO₃ and 0.1% NH₃·H₂O) and CH₃CN (45% CH₃CN up to 85% in 10 min); Detector, UV. This resulted in 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide (20 mg, 39.9%) as a yellow solid. LC-MS (ESI, m/z) M+1: 928. ¹HNMR (400 MHz, DMSO-d₆) δ 12.31 (s, 1H), 11.11 (d, J=62.7 Hz, 1H), 8.63-8.23 (m, 2H), 7.84-7.31 (m, 6H), 7.09 (dt, J=14.8, 2.8 Hz, 1H), 6.95-6.53 (m, 4H), 6.02 (ddd, J=12.1, 3.4, 1.8 Hz, 1H), 4.70-4.54 (m, 1H), 4.32 (s, 2H), 3.93-3.47 (m, 6H), 3.31-2.92 (m, 10H), 2.46-2.15 (m, 2H), 1.87 (s, 4H), 1.72-1.14 (m, 12H).

### Example 27: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-{2-[(2,2,2-trifluoroethyl)amino]phenyl}pyrrolidin-1-yl]-7-azaspiro[3.5]nonan-7-yl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at room temperature. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), DCM (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-{2-[(2,2,2-trifluoroethyl)amino]phenyl}pyrrolidine-1-carboxylate: Into a 40 mL vial were added tert-butyl (2S)-2-(2-bromophenyl)pyrrolidine-1-carboxylate (1.5 g, 4.5 mmol, 1.0 eq), 2,2,2-trifluoroethylamine (1.3 g, 13.5 mmol, 3.0 eq), toluene (20 mL), t-BuONa (1.3 g, 13.5 mmol, 3.0 eq), Chloro[(diadamantan-1-yl)(n-butyl)phosphino][2-aminao-1,1-biphenyl-2-yl]palladium(II) (302 mg, 0.4 mmol, 0.1 eq). The resulting mixture was stirred for overnight at 110° C. under nitrogen atmosphere. The mixture was allowed to cool down to 25° C. The resulting mixture was diluted with water (200 mL). The resulting mixture was extracted with Ethyl acetate (2×100 mL). The combined organic layers were washed with brine (1×200 mL), dried over anhydrous Na₂SO₄. After filtration, the filtrate was concentrated under reduced pressure. The residue was purified by reverse flash chromatography with the following conditions: column, C18 silica gel; mobile phase, CN₃CN in water (0.05% TFA), 30% to 90% gradient in 10 min; detector, UV 220 nm. This resulted in tert-butyl (2S)-2-{2-[(2,2,2-trifluoroethyl)amino]phenyl}pyrrolidine-1-carboxylate (1.2 g, 76.7%) as a white solid. LC-MS (ESI, m/z) M+1: 345.

Synthesis of 2-[(2S)-pyrrolidin-2-yl]-N-(2,2,2-trifluoroethyl)aniline hydrochloride: A solution of tert-butyl (2S)-2-{2-[(2,2,2-trifluoroethyl)amino]phenyl}pyrrolidine-1-carboxylate (640 mg, 1.8 mmol, 1.0 eq) in HCl (gas) in 1,4-dioxane (10 mL) was stirred for 1 hour at 25° C. The resulting mixture was concentrated under vacuum. This resulted in 2-[(2S)-pyrrolidin-2-yl]-N-(2,2,2-trifluoroethyl)aniline hydrochloride (520 mg, 99.6%) as a white solid. LC-MS (ESI, m/z) M+1: 245. ¹HNMR (400 MHz, DMSO-d₆) δ 10.07 (s, 1H), 8.88 (s, 1H), 7.44 (dd, J=7.8, 1.7 Hz, 1H), 7.30-7.19 (m, 1H), 6.91 (d, J=8.3 Hz, 1H), 6.78 (t, J=7.4 Hz, 1H), 6.26 (d, J=6.7 Hz, 1H), 4.66 (t, J=7.7 Hz, 1H), 4.12-3.86 (m, 1H), 3.62 (d, J=18.4 Hz, 1H), 3.37-3.26 (m, 2H), 2.37-2.21 (m, 1H), 2.17-1.91 (m, 3H).

Synthesis of N-{3-nitro-4-[(oxan-4-ylmethyl)amino]benzenesulfonyl}-4-{2-[(2S)-2-{2-[(2,2,2-trifluoroethyl)amino]phenyl}pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (150 mg, 0.2 mmol, 1.0 eq), CH₂Cl₂ (3 mL), 2-[(2S)-pyrrolidin-2-yl]-N-(2,2,2-trifluoroethyl)aniline hydrochloride (47 mg, 0.2 mmol, 1.0 eq), NaBH(OAc)₃ (71 mg, 0.3 mmol, 2.0 eq). The resulting mixture was stirred for overnight at 40° C. After cooling to 25° C. The residue was purified by Prep-TLC (dichloromethane/methanol=13: 1) to afford N-{3-nitro-4-[(oxan-4-ylmethyl)amino]benzenesulfonyl}-4-{2-[(2S)-2-{2-[(2,2,2-trifluoroethyl)amino]phenyl}pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (150 mg, 79.8%) as a yellow solid. LC-MS (ESI, m/z) M+1: 1130.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-{2-[(2,2,2-trifluoroethyl)amino]phenyl}pyrrolidin-1-yl]-7-azaspiro[3.5]nonan-7-yl}benzamide: Into a 100 mL round-bottom flask were added N-{3-nitro-4-[(oxan-4-ylmethyl)amino]benzenesulfonyl}-4-{2-[(2S)-2-{2-[(2,2,2-trifluoroethyl)amino]phenyl}pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (150 mg, 0.1 mmol, 1.0 eq), 1.0 M TBAF in THE (5 mL), ethylenediamine (160 mg, 2.6 mmol, 20.0 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃ and 0.1% NH₃·H₂O) and CH₃CN (45% CH₃CN up to 85% in 10 min); Detector, UV. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-{2-[(2,2,2-trifluoroethyl)amino]phenyl}pyrrolidin-1-yl]-7-azaspiro[3.5]nonan-7-yl}benzamide (13 mg, 9.8%) as a yellow solid. LC-MS (ESI, m/z) M+1: 1000. ¹HNMR (400 MHz, DMSO-d₆) δ 11.10 (d, J=53.7 Hz, 1H), 8.66-8.23 (m, 2H), 7.50 (d, J=40.1 Hz, 3H), 7.15-6.99 (m, 2H), 6.97-6.51 (m, 6H), 6.07-5.96 (m, 1H), 4.63 (s, 1H), 4.29 (s, 1H), 4.06 (q, J=9.0, 8.5 Hz, 2H), 3.94-3.75 (m, 3H), 3.55 (t, J=9.4 Hz, 1H), 3.30-2.67 (m, 12H), 2.14 (d, J=8.8 Hz, 1H), 1.86 (d, J=32.2 Hz, 6H), 1.64 (d, J=11.8 Hz, 3H), 1.55-1.17 (m, 8H).

### Example 28: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(propane-2-sulfonyl)phenyl]pyrrolidin-1-yl]-7-azaspiro[3.5]nonan-7-yl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N₁,N₂-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of 2-(isopropylsulfanyl)benzaldehyde: Into a 500 mL round-bottom flask were added 2-fluor-benzaldehyd (15.0 g, 120.8 mmol, 1.0 eq), DMF (150 mL), K₂CO₃ (20.0 g, 144.7 mmol, 1.2 eq), 2-propanethiol (10.0 g, 131.3 mmol, 1.1 eq). The resulting mixture was stirred for overnight at 70° C. The mixture was allowed to cool down to 25° C. The resulting mixture was diluted with water (500 mL). The resulting mixture was extracted with Ethyl acetate (2×200 mL). The combined organic layers were washed with brine (1×500 mL), dried over anhydrous Na₂SO₄. After filtration, the filtrate was concentrated under reduced pressure. This resulted in 2-(isopropylsulfanyl)benzaldehyde (17 g, 35.1%) as a yellow oil. LC-MS (ESI, m/z) M+1: 181.

Synthesis of (S)—N-{[2-(isopropylsulfanyl)phenyl]methylidene}-2-methylpropane-2-sulfinamide: Into a 500 mL round-bottom flask were added 2-(isopropylsulfanyl)benzaldehyde (17.0 g, 94.3 mmol, 1.0 eq), (S)-2-methylpropane-2-sulfinamide (11.4 g, 94.3 mmol, 1.0 eq), THE (200 mL), Ti(OEt)₄ (43.0 g, 188.6 mmol, 2.0 eq). The resulting mixture was stirred for 2 hours at 55° C. The mixture was allowed to cool down to 25° C. The resulting mixture was diluted with water (500 mL). The resulting mixture was extracted with Ethyl acetate (2×200 mL). The combined organic layers were washed with brine (1×500 mL), dried over anhydrous Na₂SO₄. After filtration, the filtrate was concentrated under reduced pressure. The crude product was purified by reverse phase flash with the following conditions (90% CH₃CN in water (0.05% TFA)) to afford (S)—N-{[2-(isopropylsulfanyl)phenyl]methylidene}-2-methylpropane-2-sulfinamide (12 g, 43.5%) as a yellow oil. ¹HNMR (400 MHz, Chloroform-d) δ 9.25 (s, 1H), 8.02 (dd, J=7.7, 1.6 Hz, 1H), 7.55 (dd, J=7.8, 1.4 Hz, 1H), 7.45 (td, J=7.6, 1.7 Hz, 1H), 7.35 (td, J=7.6, 1.4 Hz, 1H), 1.35-1.26 (m, 15H).

Synthesis of (S)—N-[(1S)-3-(1,3-dioxan-2-yl)-1-[2-(isopropylsulfanyl)phenyl]propyl]-2-methylpropane-2-sulfinamide: Into a 500-mL 3-necked round-bottom flask purged and maintained with an inert atmosphere of nitrogen, were placed Mg (4.0 g, 164.3 mmol, 4.0 eq), I₂ (1.0 g, 4.1 mmol, 0.1 eq), THE (100 mL), the resulting solution was warmed to 65° C., followed by the addition of 2-(2-bromoethyl)-1,3-dioxane (3.2 g, 16.4 mmol, 0.4 eq) in THE (20 mL) at 65° C. After addition completed, the mixture was cooled to 25° C., another 2-(2-bromoethyl)-1,3-dioxane (28.7 g, 148.0 mmol, 3.6 eq) in THE (110 mL) was added. To the above solution was added a solution of (S)—N-{[2-(isopropylsulfanyl)phenyl]methylidene}-2-methylpropane-2-sulfinamide (11.6 g, 41.1 mmol, 1.0 eq) in CH₂Cl₂ (50 mL) dropwise at −40° C. The resulting solution was stirred for 1 hour at −40° C. in a liquid nitrogen bath. The reaction was then quenched by the addition of aqueous NH₄Cl (500 mL). The resulting solution was extracted with dichloromethane (3×300 mL) and the organic layers combined. The resulting mixture was washed with brine (1×1000 mL). The mixture was dried over anhydrous sodium sulfate. After filtration, the filtrate was concentrated under reduced pressure. The crude residue was purified by a flash column (silica gel, ethyl acetate/petroleum ether=1:1) to afford (S)—N-[(1S)-3-(1,3-dioxan-2-yl)-1-[2-(isopropylsulfanyl)phenyl]propyl]-2-methylpropane-2-sulfinamide (15 g, 91.4%) as a light yellow oil. LC. MS (ESI, m/z) M+1: 400.

Synthesis of (2S)-2-[2-(isopropylsulfanyl)phenyl]pyrrolidine: Into a 500-mL round-bottom flask, were placed (S)—N-[(1S)-3-(1,3-dioxan-2-yl)-1-[2-(isopropylsulfanyl)phenyl]propyl]-2-methylpropane-2-sulfinamide (15.0 g, 37.5 mmol, 1.0 eq), TFA (100 mL), water (25 mL), the resulting solution was stirred for 1 hour at 25° C. TFA (100 mL), Et₃SiH (13.1 g, 112.6 mmol, 3.0 eq) was added. The resulting solution was stirred overnight at 25° C. The resulting mixture was concentrated. The resulting solution was diluted with water (500 mL). The resulting solution was extracted with MTBE (2×200 mL) and the organic layers combined. The resulting mixture was washed with 2.0 M HCl (1×500 mL). The pH value of the aqueous was adjusted to 13 with NaOH (4 M). The resulting solution was extracted with dichloromethane (3×300 mL) and the organic layers combined and dried over anhydrous sodium sulfate. After filtration, the filtrate was concentrated under reduced pressure. This resulted in (2S)-2-[2-(isopropylsulfanyl)phenyl]pyrrolidine (1 g, 12.03%) as yellow oil. ¹HNMR (400 MHz, DMSO-d₆) δ 7.64-7.57 (m, 1H), 7.40-7.34 (m, 1H), 7.23-7.15 (m, 2H), 4.46 (t, J=7.5 Hz, 1H), 3.46 (p, J=6.6 Hz, 1H), 3.03 (ddd, J=9.7, 7.1, 5.4 Hz, 1H), 2.90 (dt, J=9.6, 7.4 Hz, 1H), 2.65 (s, 1H), 2.15 (dtd, J=12.2, 7.8, 5.8 Hz, 1H), 1.80-1.65 (m, 2H), 1.24 (dd, J=17.1, 6.7 Hz, 6H).

Synthesis of 4-{2-[(2S)-2-[2-(isopropylsulfanyl)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 100 mL round-bottom flask were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (2.0 g, 2.3 mmol, 1.0 eq), (2S)-2-[2-(isopropylsulfanyl)phenyl]pyrrolidine (500 mg, 2.3 mmol, 1.0 eq), CH₂Cl₂ (20 mL), NaBH(OAc)₃ (959 mg, 4.5 mmol, 2.0 eq). The resulting mixture was stirred for overnight at 40° C. The resulting mixture was concentrated under vacuum. The crude residue was purified by a flash column (silica gel, dichloromethane/methanol=12: 1) to afford 4-{2-[(2S)-2-[2-(isopropylsulfanyl)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (2 g, 63.9%) as a yellow solid. LC-MS (ESI, m/z) M+1: 1107.

Synthesis of N-{3-nitro-4-[(oxan-4-ylmethyl)amino]benzenesulfonyl}-4-{2-[(2S)-2-[2-(propane-2-sulfonyl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 4-{2-[(2S)-2-[2-(isopropylsulfanyl)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (301 mg, 0.3 mmol, 1.0 eq), CH₂Cl₂ (5 mL), m-CPBA (83 mg, 0.4 mmol, 1.5 eq). The resulting mixture was stirred for 1 hour at 25° C. The resulting mixture was diluted with water (200 mL). The resulting mixture was extracted with dichloromethane (2×50 mL). The combined organic layers were washed with aqueous Na₂SO₃ (1×200 mL), dried over anhydrous Na₂SO₄. After filtration, the filtrate was concentrated under reduced pressure. The residue was purified by Prep-TLC (dichloromethane/methanol=12: 1) to afford N-{3-nitro-4-[(oxan-4-ylmethyl)amino]benzenesulfonyl}-4-{2-[(2S)-2-[2-(propane-2-sulfonyl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (170 mg, 54.9%) as a yellow solid. LC-MS (ESI, m/z) M+1: 1139.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(propane. 2-sulfonyl)phenyl]pyrrolidin-1-yl]-7-azaspiro[3.5]nonan-7-yl}benzamide: Into a 100 mL round-bottom flask were added N-{3-nitro-4-[(oxan-4-ylmethyl)amino]benzenesulfonyl}-4-{2-[(2S)-2-[2-(propane-2-sulfonyl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (170 mg, 0.1 mmol, 1.0 eq), TBAF in THE (1.0 M, 5 mL), ethylenediamine (180 mg, 2.9 mmol, 20.0 eq). The resulting mixture was stirred for 4 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃ and 0.1% NH₃·H₂O) and CH₃CN (45% CH₃CN up to 85% in 10 min); Detector, UV. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}-4-{2-[(2S)-2-[2-(propane-2-sulfonyl)phenyl]pyrrolidin-1-yl]-7-azaspiro[3.5]nonan-7-yl}benzamide (15 mg, 9.9%) as a yellow solid. LC-MS (ESI, m/z) M+1: 1009. ¹HNMR (400 MHz, DMSO-d₆) ¹H NMR (400 MHz, DMSO-d₆) δ 11.03 (d, J=17.6 Hz, 2H), 8.31 (dd, J=19.6, 2.1 Hz, 4H), 8.06 (s, 1H), 7.73 (s, 1H), 7.64-7.34 (m, 5H), 7.14 (s, 1H), 7.05 (d, −3.8 Hz, 2H), 4.86 (s, 1H), 4.70 (s, 1H), 4.50-4.39 (m, 1H), 4.30-4.00 (m, 2H), 3.92-3.70 (m, 3H), 3.55 (s, 2H), 3.43 (t, J=8.9 Hz, 3H), 3.26 (s, 3H), 3.21-3.06 (m, 4H), 3.06-2.72 (m, 3H), 2.41 (s, 1H), 2.35-2.17 (m, 4H), 2.08 (s, 3H), 1.88 (s, 2H), 1.77-1.20 (m, 2H), 1.15 (q, −6.1 Hz, 4H), 1.06-0.95 (m, 3H).

### Example 29: Preparation of 4-{2-[(2S)-2-[2-(dimethylphosphoryl)phenyl]pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N₁,N₂-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), CH₂Cl₂ (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-[2-(dimethylphosphoryl)phenyl]pyrrolidine-1-carboxylate: Into a 100 mL round-bottom flask were added tert-butyl (2S)-2-(2-bromophenyl)pyrrolidine-1-carboxylate (1.0 g, 3.1 mmol, 1.0 eq) and (methylphosphonoyl)methane (360 mg, 4.6 mmol, 1.5 eq), dioxane (10 mL), Cs₂CO₃ (2.0 g, 6.1 mmol, 2.0 eq), Binap Palladacycle Gen. 2 (286 mg, 0.3 mmol, 0.1 eq). The resulting mixture was stirred for overnight at 110° C. under nitrogen atmosphere. The mixture was allowed to cool down to 25° C. The resulting mixture was concentrated under vacuum. The crude product was purified by reverse phase flash with the following conditions (40% CH₃CN in water (0.05% TFA)) to afford tert-butyl (2S)-2-[2-(dimethylphosphoryl)phenyl]pyrrolidine-1-carboxylate (174 mg, 17.5%) as a yellow oil. ¹HNMR (400 MHz, Chloroform-d) δ 7.60-7.30 (m, 4H), 5.51 (t, J=6.3 Hz, 1H), 3.69 (s, 2H), 2.59 (s, 1H), 2.15-1.81 (m, 9H), 1.44 (s, 4H), 1.17 (s, 5H).

Synthesis of (2S)-2-[2-(dimethylphosphoryl)phenyl]pyrrolidine hydrochloride: A solution of tert-butyl (2S)-2-[2-(dimethylphosphoryl)phenyl]pyrrolidine-1-carboxylate (170 mg, 0.5 mmol, 1.0 eq) in HCl (gas) in 1,4-dioxane (5 mL) was stirred for 1 hour at 25° C. The resulting mixture was concentrated under vacuum. This resulted in (2S)-2-[2-(dimethylphosphoryl)phenyl]pyrrolidine hydrochloride (130 mg, 95.22%) as a yellow oil. LC-MS (ESI, m/z) M+1: 224. ¹HNMR (400 MHz, Chloroform-d) δ 10.45 (s, 1H), 9.91 (s, 1H), 7.90 (d, J=7.4 Hz, 1H), 7.64 (d, J=7.5 Hz, 1H), 7.51 (s, 2H), 5.99 (d, J=12.6 Hz, 2H), 4.96 (s, 1H), 3.71-3.58 (m, 1H), 3.39 (d, J=10.1 Hz, 1H), 2.40 (d, J=27.0 Hz, 3H), 2.16 (s, 1H), 2.06-1.79 (m, 6H).

Synthesis of 4-{2-[(2S)-2-[2-(dimethylphosphoryl)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (150 mg, 0.2 mmol, 1.0 eq), (2S)-2-[2-(dimethylphosphoryl)phenyl]pyrrolidine hydrochloride (43 mg, 0.2 mmol, 1.0 eq), CH₂Cl₂ (5 mL), NaBH(OAc)₃ (71 mg, 0.4 mmol, 2.0 eq). The resulting mixture was stirred for overnight at 40° C. The mixture was allowed to cool down to 25° C. The resulting mixture was concentrated under vacuum. The residue was purified by Prep-TLC (dichloromethane/methanol=12: 1) to afford 4-{2-[(2S)-2-[2-(dimethylphosphoryl)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (150 mg, 81.3%) as a yellow solid. LC-MS (ESI, m/z) M+1: 1109.

Synthesis of 4-{2-[(2S)-2-[2-(dimethylphosphoryl)phenyl]pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen. 10-yl]-N-{3-nitro-4-[(oxan-4-ylmethyl)amino]benzenesulfonyl}benzamide: Into a 100 mL round-bottom flask were added 4-{2-[(2S)-2-[2-(dimethylphosphoryl)phenyl]pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (150 mg, 0.1 mmol, 1.0 eq), TBAF in THE (1.0 M, 5 mL), ethylenediamine (163 mg, 2.7 mmol, 20.0 eq). The resulting mixture was stirred for 3 hours at 70° C. The mixture was allowed to cool down to 25° C. The resulting mixture was diluted with water (50 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃ and 0.1% NH₃·H₂O) and CH₃CN (45% CH₃CN up to 85% in 10 min); Detector, UV. This resulted in 4-{2-[(2S)-2-[2-(dimethylphosphoryl)phenyl]pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide (12 mg, 9.0%) as a yellow solid. LC-MS (ESI, m/z) M+1: 979. ¹HNMR (400 MHz, DMSO-d₆) δ 12.28 (s, 1H), 11.09 (d, J=48.7 Hz, 1H), 8.64-8.19 (m, 2H), 7.86 (s, 1H), 7.55 (d, J=11.6 Hz, 3H), 7.30 (s, 1H), 7.13-6.99 (m, 1H), 6.69 (dd, J=61.2, 23.4 Hz, 4H), 6.06-5.94 (m, 1H), 4.63 (s, 1H), 4.28 (s, 2H), 3.99-3.66 (m, 4H), 3.48 (dt, J=37.7, 8.8 Hz, 2H), 3.18 (dd, J=49.5, 14.3 Hz, 9H), 2.27 (s, 2H), 2.10-1.12 (m, 24H).

### Example 30: Preparation of 4-{2-[(2S)-2-(2-carbamoylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide

Synthesis of tert-butyl 2-[(2S)-2-(2-cyanophenyl)pyrrolidin-1-yl]-7-azaspiro[3.5]nonane-7-carboxylate: Into a 40 mL vial were added tert-butyl 2-[(2S)-2-(2-bromophenyl)pyrrolidin-1-yl]-7-azaspiro[3.5]nonane-7-carboxylate (1.0 g, 2.2 mmol, 1.0 eq), Zn(CN)₂ (792 mg, 6.7 mmol, 3.0 eq), Zn (292 mg, 4.4 mmol, 2.0 eq), DMA (10 mL), dppf (253 mg, 0.5 mmol, 0.2 eq), Pd₂(dba)₃ (209 mg, 0.2 mmol, 0.1 eq). The resulting mixture was stirred for overnight at 100° C. under nitrogen atmosphere. The mixture was allowed to cool down to 25° C. The resulting mixture was diluted with water (300 mL). The resulting mixture was extracted with Ethyl acetate (2×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=2: 3) to afford tert-butyl 2-[(2S)-2-(2-cyanophenyl)pyrrolidin-1-yl]-7-azaspiro[3.5]nonane-7-carboxylate (820 mg, 93.2%) as a yellow oil. LC-MS (ESI, m/z) M+1: 396.

Synthesis of 2-[(2S)-1-{7-azaspiro[3.5]nonan-2-yl}pyrrolidin-2-yl]benzonitrile hydrochloride: A solution of tert-butyl 2-[(2S)-2-(2-cyanophenyl)pyrrolidin-1-yl]-7-azaspiro[3.5]nonane-7-carboxylate (820 mg, 2.1 mmol, 1.0 eq) in HCl (gas) in 1,4-dioxane (10 mL) was stirred for 1 hour at 25° C. The resulting mixture was concentrated under vacuum. This resulted in 2-[(2S)-1-{7-azaspiro[3.5]nonan-2-yl}pyrrolidin-2-yl]benzonitrile hydrochloride (700 mg, crude) as a light yellow solid. LC-MS (ESI, m/z) M+1: 296.

Synthesis of methyl 2-bromo-4-{2-[(2S)-2-(2-cyanophenyl)pyrrolidin-1-yl]-7-azaspiro[3.5]nonan. 7-yl}benzoate: Into a 40 mL round-bottom flask were added 2-[(2S)-1-{7-azaspiro[3.5]nonan-2-yl}pyrrolidin-2-yl]benzonitrile hydrochloride (700 mg, 2.1 mmol, 1.0 eq), DMSO (10 mL), methyl 2-bromo-4-fluorobenzoate (492 mg, 2.1 mmol, 1.0 eq), Na₂CO₃ (447 mg, 4.2 mmol, 2.0 eq). The resulting mixture was stirred for overnight at 110° C. The mixture was allowed to cool down to 25° C. The resulting mixture was diluted with water (300 mL). The resulting mixture was extracted with Ethyl acetate (2×100 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1:1) to afford methyl 2-bromo-4-{2-[(2S)-2-(2-cyanophenyl)pyrrolidin-1-yl]-7-azaspiro[3.5]nonan-7-yl}benzoate (670 mg, 62.4%) as a yellow oil. LC-MS (ESI, m/z) M+1: 508. ¹HNMR (400 MHz, Chloroform-d) δ 7.78 (dd, J=18.3, 8.4 Hz, 2H), 7.59 (qd, J=7.7, 1.3 Hz, 2H), 7.33 (td, J=7.6, 1.2 Hz, 1H), 7.06 (d, J=2.5 Hz, 1H), 6.74 (dd, J=9.0, 2.6 Hz, 1H), 3.87 (s, 3H), 3.83 (t, J=7.9 Hz, 1H), 3.29-3.05 (m, 6H), 2.55-2.29 (m, 2H), 1.97 (ddt, J=9.9, 7.2, 3.5 Hz, 2H), 1.91-1.47 (m, 9H), 1.39-1.25 (m, 2H).

Synthesis of methyl 4-{2-[(2S)-2-(2-cyanophenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate: Into a 40 mL vial were added methyl 2-bromo-4-{2-[(2S)-2-(2-cyanophenyl)pyrrolidin-1-yl]-7-azaspiro[3.5]nonan-7-yl}benzoate (640 mg, 1.3 mmol, 1.3 eq), (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (347 mg, 1.0 mmol, 1.0 eq), toluene (10 mL), Cs₂CO₃ (975 mg, 3.0 mmol, 3.0 eq), Xantphos (115 mg, 0.2 mmol, 0.2 eq), Pd₂(dba)₃·CHCl₃ (103 mg, 0.1 mmol, 0.1 eq). The resulting mixture was stirred for 2 hours at 100° C. under nitrogen atmosphere. The mixture was allowed to cool down to 25° C. The resulting mixture was concentrated under vacuum. The crude residue was purified by a flash column (silica gel, ethyl acetate/petroleum ether=3:2) to afford methyl 4-{2-[(2S)-2-(2-cyanophenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (580 mg, 74.9%) as a yellow solid. LC-MS (ESI, m/z) M+1: 775.

Synthesis of methyl 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoate: Into a 100 mL round-bottom flask were added methyl 4-{2-[(2S)-2-(2-cyanophenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (550 mg, 0.7 mmol, 1.0 eq), TBAF in THE (1.0 M, 10 mL), ethylenediamine (640 mg, 10.6 mmol, 15.0 eq). The resulting mixture was stirred for 6 hours at 70° C. The mixture was allowed to cool down to 25° C. The resulting mixture was diluted with water (200 mL). The resulting mixture was extracted with Ethyl acetate (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate) to afford methyl 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoate (237 mg, 51.8%) as a light yellow solid. LC-MS (ESI, m/z) M+1: 645.

Synthesis of 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoic acid: Into a 100 mL round-bottom flask were added methyl 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoate (230 mg, 0.4 mmol, 1.0 eq), MeOH (5 mL), dioxane (5 mL), 4 M NaOH (1 mL). The resulting mixture was stirred for overnight at 40° C. The mixture was allowed to cool down to 25° C. The resulting mixture was concentrated under vacuum. The mixture was acidified to pH 5 with 2 N HCl. The resulting mixture was extracted with dichloromethane (2×50 mL). The combined organic layers were washed with brine (1×100 mL), dried over anhydrous Na₂SO₄. After filtration, the filtrate was concentrated under reduced pressure. The crude residue was purified by a flash column (silica gel, dichloromethane/methanol=10: 1) to afford 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoic acid (100 mg, 44.4%) as a yellow solid. LC-MS (ESI, m/z) M+1: 631.

Synthesis of 4-{2-[(2S)-2-(2-carbamoylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoic acid: Into a 20 mL vial were added 4-{2-[(2S)-2-(2-cyanophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoic acid (65 mg, 0.1 mmol, 1.0 eq), EtOH (6 mL), water (3 mL), platinum ion dimethylphosphinous acid didimethylphosphinite (5 mg, 0.01 mmol, 0.1 eq). The resulting mixture was stirred for overnight at 80° C. The mixture was allowed to cool down to 25° C. The resulting mixture was concentrated under vacuum. The residue was purified by Prep-TLC (dichloromethane/methanol=5:1) to afford 4-{2-[(2S)-2-(2-carbamoylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoic acid (30 mg, 44.8%) as a light yellow solid. LC-MS (ESI, m/z) M+1: 649.

Synthesis of 4-{2-[(2S)-2-(2-carbamoylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide: Into a 20 mL vial were added 4-{2-[(2S)-2-(2-carbamoylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]benzoic acid (30 mg, 0.05 mmol, 1.0 eq), 3-nitro-4-[(oxan-4-ylmethyl)amino]benzenesulfonamide (15 mg, 0.05 mmol, 1.0 eq), CH₂Cl₂ (3 mL), EDCI (18 mg, 0.1 mmol, 2.0 eq), DMAP (23 mg, 0.2 mmol, 4.0 eq). The resulting mixture was stirred for overnight at 30° C. The resulting mixture was concentrated under vacuum. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃ and 0.1% NH₃·H₂O) and CH₃CN (45% CH₃CN up to 85% in 10 min); Detector, UV. This resulted in 4-{2-[(2S)-2-(2-carbamoylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide (10 mg, 22.8%) as a yellow solid. LC-MS (ESI, m/z) M+1: 946. ¹HNMR (400 MHz, DMSO-d₆) δ 12.3 (s, 1H), 11.11 (d, J=62.7 Hz, 1H), 8.32 (d, J=16.6 Hz, 3H), 8.08-7.80 (m, 1H), 7.77-7.14 (m, 8H), 7.14-7.00 (m, 1H), 6.69 (dd, J=59.5, 23.4 Hz, 2H), 6.01 (d, J=14.8 Hz, 1H), 4.65 (s, 1H), 4.26 (s, 1H), 4.08 (t, J=7.1 Hz, 1H), 3.87 (d, J=11.1 Hz, 4H), 3.07 (s, 6H), 2.23 (d, J=27.6 Hz, 3H), 2.00-1.11 (m, 21H).

### Example 31: Preparation of 4-{2-[(2S)-2-{2-[(4S)-2,2-dimethyloxan-4-yl]phenyl}pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide (assumed)

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), ethyl acetate (20 mL) and 2.0 M HCl in ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N₁,N₂-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), DCM (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of N′-[(4E)-2,2-dimethyloxan-4-ylidene]-4-methylbenzenesulfonohydrazide: Into a 500 mL round-bottom flask were added 2,2-dimethyloxan-4-one (5 g, 39.0 mmol, 1.0 eq) and 4-toluenesulfonyl hydrazide (7.2 g, 39.0 mmol, 1.0 eq) in dioxane (100 mL). The resulting mixture was stirred for 2 hours at 80° C. The mixture was allowed to cool down to 25° C. The resulting mixture was concentrated under vacuum. The crude product was re-crystallized from ethyl acetate/petroleum ether c (1:20) to afford N′-[(4E)-2,2-dimethyloxan-4-ylidene]-4-methylbenzenesulfonohydrazide (11 g, 95.1%) as light yellow solid. ¹HNMR (300 MHz, DMSO-de) δ 10.24 (s, 1H), 7.77-7.68 (m, 2H), 7.39 (d, J=8.0 Hz, 2H), 3.62 (d, J=11.8 Hz, 1H), 2.36 (d, J=12.5 Hz, 5H), 2.10 (s, 2H), 1.02 (s, 6H).

Synthesis of tert-butyl (2S)-2-[2-(2,2-dimethyl-5,6-dihydropyran-4-yl)phenyl]pyrrolidine-1-carboxylate: Into a 250 mL round-bottom flask were added tert-butyl (2S)-2-(2-bromophenyl)pyrrolidine-1-carboxylate (3 g, 9.2 mmol, 1.0 eq) and N′-[(4E)-2,2-dimethyloxan-4-ylidene]-4-methylbenzenesulfonohydrazide (8.2 g, 27.7 mmol, 3.0 eq), dioxane (50 mL), t-BuOLi (2.2 g, 27.7 mmol, 3.0 eq), Chloro[(diadamantan-1-yl)(n-butyl)phosphino][2-aminao-1,1-biphenyl-2-yl]palladium(II) (616 mg, 0.9 mmol, 0.1 eq). The resulting mixture was stirred for overnight at 110° C. under nitrogen atmosphere. The mixture was allowed to cool down to 25° C. The resulting mixture was diluted with water (300 The resulting mixture was extracted with Ethyl acetate (2×100 mL). The combined organic layers were washed with brine (1×300 mL), dried over anhydrous Na₂SO₄. After filtration, the filtrate was concentrated under reduced pressure. The residue/crude product was purified by reverse phase flash with the following conditions (80% CH₃CN in water (0.05% TFA)) to afford tert-butyl (2S)-2-[2-(2,2-dimethyl-5,6-dihydropyran-4-yl)phenyl]pyrrolidine-1-carboxylate (2 g, 66.9%) as a yellow solid. ¹HNMR (300 MHz, Chloroform-d) δ 7.22 (ddd, J=7.0, 4.9, 1.9 Hz, 2H), 7.15 (t, J=7.0 Hz, 1H), 7.06 (ddd, J=7.4, 3.2, 1.8 Hz, 1H), 5.58 (d, J=31.1 Hz, 1H), 4.94 (s, 1H), 4.34 (q, J=2.7 Hz, 1H), 3.96 (t, J=5.4 Hz, 1H), 3.78-3.51 (m, 2H), 2.50-2.13 (m, 5H), 1.85 (dtd, J=23.2, 15.1, 14.6, 7.0 Hz, 4H), 1.36 (d, J=2.4 Hz, 9H), 1.25-1.07 (m, 6H).

Synthesis of tert-butyl (2R)-2-{2-[(4R)-2,2-dimethyloxan-4-yl]phenyl}pyrrolidine-1-carboxylate(assumed) and tert-butyl (2R)-2-{2-[(4S)-2,2-dimethyloxan-4-yl]phenyl}pyrrolidine-1-carboxylate (assumed)) Into a 100 mL round-bottom flask were added MeOH (20 mL) and tert-butyl (2R)-2-[2-(2,2-dimethyl-5,6-dihydropyran-4-yl)phenyl]pyrrolidine-1-carboxylate (500 mg, 1.4 mmol, 1.0 eq), Pd(OH)₂/C (20 mg, 0.1 mmol, 0.1 eq) and at 25° C. The resulting mixture was stirred for 12 hours at 25° C. under hydrogen (3 atm) atmosphere. The resulting mixture was filtered. the filter cake was washed with MeOH (50 mL). The crude product (450 mg) was purified by Prep-HPLC with the following conditions: Column, SunFire Prep C18 OBD Column, 19*150 mm, 5 μm 10 nm; mobile phase, water (0.05% TFA) and CH₃CN (30% CH₃CN up to 95% in 7 min); Detector, UV 220 to afford tert-butyl (2R)-2-{2-[(4R)-2,2-dimethyloxan-4-yl]phenyl}pyrrolidine-1-carboxylate(assumed) (200 mg, 39.7%) as white solid. and tert-butyl (2R)-2-{2-[(4S)-2,2-dimethyloxan-4-yl]phenyl}pyrrolidine-1-carboxylate(assumed) (200 mg, 39.7%) as white solid. 3A: LC-MS (ESI, m/z) M+1:360. ¹HNMR (300 MHz, DMSO-d₆) δ 7.29-7.11 (m, 3H), 7.01 (d, J=7.5 Hz, 1H), 5.02 (s, 1H), 3.74 (d, J=8.3 Hz, 2H), 3.60 (dt, J=10.5, 6.6 Hz, 1H), 3.53-3.40 (m, 1H), 3.16 (t, J=12.3 Hz, 1H), 2.39 (d, J=9.7 Hz, 1H), 1.84 (q, J=6.1 Hz, 2H), 1.75-1.48 (m, 4H), 1.42 (d, J=20.7 Hz, 4H), 1.29 (s, 3H), 1.16 (s, 3H), 1.08 (s, 6H). 3B: LC-MS (ESI, m/z) M+1:360. ¹HNMR (300 MHz, DMSO-d₆) δ 7.26 (d, J=7.5 Hz, 1H), 7.23-7.09 (m, 2H), 7.00 (d, J=6.8 Hz, 1H), 5.16 (d, J=22.6 Hz, 1H), 3.87-3.65 (m, 2H), 3.59 (ddd, J=10.6, 7.2, 5.1 Hz, 1H), 3.48 (dt, J=10.5, 7.5 Hz, 1H), 3.23 (s, 1H), 2.47-2.24 (m, 2H), 1.82 (q, J=6.4 Hz, 2H), 1.54 (d, J=22.4 Hz, 5H), 1.38 (d, J=7.8 Hz, 3H), 1.28 (s, 3H), 1.18 (s, 3H), 1.07 (s, 7H).

Synthesis of (2R)-2-{2-[(4S)-2,2-dimethyloxan-4-yl]phenyl}pyrrolidine(assumed): Into a 50 mL round-bottom flask were added tert-butyl (2R)-2-{2-[(4S)-2,2-dimethyloxan-4-yl]phenyl}pyrrolidine-1-carboxylate(assumed) (75 mg, 0.2 mmol, 1.00 eq), CH₂Cl₂(1.5 mL) and TFA (0.5 mL) at 25° C. The resulting mixture was stirred for 3 hours at 25° C. The resulting mixture was concentrated under reduced pressure. This resulted in (2R)-2-{2-[(4S)-2,2-dimethyloxan-4-yl]phenyl}pyrrolidine (55 mg, crude) as light yellow oil. LC-MS (ESI, m/z) M+1: 260.

Synthesis of 4-{2-[(2S)-2-{2-[(4S)-2,2-dimethyloxan-4-yl]phenyl}pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide(assumed): Into a 8 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide(assumed) (60 mg, 0.07 mmol, 1.0 eq) and (2S)-2-{2-[(4S)-2,2-dimethyloxan-4-yl]phenyl}pyrrolidine (55 mg, 0.2 mmol, 3.0 eq), NaBH(OAc)₃ (28 mg, 0.1 mmol, 2 eq) at 25° C. The resulting mixture was stirred for 16 hours at 25° C. The reaction was quenched by the addition of MeOH (2.0 mL) at 25° C. The resulting mixture was concentrated under reduced pressure. The residue was purified by TLC eluted with dichloromethane/methanol (95:5) to afford 4-{2-[(2S)-2-{2-[(4S)-2,2-dimethyloxan-4-yl]phenyl}pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide(assumed) (60 mg, 78.7%) as yellow solid. LC-MS (ESI, m/z) M+1:1145

Synthesis of 4-{2-[(2S)-2-{2-[(4S)-2,2-dimethyloxan-4-yl]phenyl}pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide Into a 8 mL vial were added 4-{2-[(2S)-2-{2-[(4S)-2,2-dimethyloxan-4-yl]phenyl}pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide(assumed) (60 mg, 0.05 mmol, 1.0 eq), 1M TBAF in tetrahydrofuran (1.0 mL) and ethylenediamine (47 mg, 0.8 mmol, 15.0 eq) at 25° C. The resulting mixture was stirred for 3 hours at 70° C. The mixture was allowed to cool down to 25° C. The resulting mixture was diluted with water (5 mL). The resulting mixture was extracted with CH₂Cl₂ (3×5 mL). The combined organic layers were washed with brine (1×5 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (3T5% CH₃CN up to 75% in 10 min); Detector, uv. The collected solution was concentrated under vacuum to remove CH₃CN and the resulting solution was dried by lyophilization. This resulted in 4-{2-[(2S)-2-{2-[(4S)-2,2-dimethyloxan-4-yl]phenyl}pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide(assumed) (11 mg, 20.6%) as yellow solid. LC-MS (ESI, m/z) M+1: 1015. ¹HNMR (300 MHz, Chloroform-d) δ 12.42 (s, 1H), 8.57 (d, J=2.3 Hz, 1H), 8.47 (s, 3H), 8.08 (d, J=9.1 Hz, 1H), 7.82 (d, J=7.4 Hz, 1H), 7.57 (s, 1H), 7.40 (s, 2H), 7.22 (s, 4H), 7.12 (d, J=3.2 Hz, 1H), 6.83 (s, 1H), 6.77-6.66 (m, 2H), 6.49 (s, 1H), 6.34 (d, J=29.2 Hz, 1H), 6.09 (s, 1H), 5.11-4.99 (m, 1H), 4.50 (t, J=7.7 Hz, 1H), 4.15-3.99 (m, 5H), 3.99-3.72 (m, 5H), 3.46 (d, J=11.9 Hz, 2H), 3.26 (dt, J=25.4, 6.2 Hz, 3H), 3.10 (s, 10H), 1.74 (d, J=12.2 Hz, 4H), 1.53-1.40 (m, 2H), 1.37-1.20 (m, 10H).

### Example 32: Preparation of 4-{2-[(2S)-2-{2-[(4R)-2,2-dimethyloxan-4-yl]phenyl}pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide) (assumed)

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), ethyl acetate (20 mL) and 2.0 M HCl in ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), DCM (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1: 15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of N′-[(4E)-2,2-dimethyloxan-4-ylidene]-4-methylbenzenesulfonohydrazide: Into a 500 mL round-bottom flask were added 2,2-dimethyloxan-4-one (5 g, 39.0 mmol, 1.0 eq) and 4-toluenesulfonyl hydrazide (7.2 g, 39.0 mmol, 1.0 eq) in dioxane (100 mL). The resulting mixture was stirred for 2 hours at 80° C. The mixture was allowed to cool down to 25° C. The resulting mixture was concentrated under vacuum. The crude product was re-crystallized from ethyl acetate/petroleum ether (1: 20) to afford N′-[(4E)-2,2-dimethyloxan-4-ylidene]-4-methylbenzenesulfonohydrazide (11 g, 95.1%) as light yellow solid. ¹HNMR (300 MHz, DMSO-de) δ 10.24 (s, 1H), 7.77-7.68 (m, 2H), 7.39 (d, J=8.0 Hz, 2H), 3.62 (d, J=11.8 Hz, 1H), 2.36 (d, J=12.5 Hz, 5H), 2.10 (s, 2H), 1.02 (s, 6H).

Synthesis of tert-butyl (2S)-2-[2-(2,2-dimethyl-5,6-dihydropyran-4-yl)phenyl]pyrrolidine-1-carboxylate: Into a 250 mL round-bottom flask were added tert-butyl (2S)-2-(2-bromophenyl)pyrrolidine-1-carboxylate (3 g, 9.2 mmol, 1.0 eq) and N′-[(4E)-2,2-dimethyloxan-4-ylidene]-4-methylbenzenesulfonohydrazide (8.2 g, 27.7 mmol, 3.0 eq), dioxane (50 mL), t-BuOLi (2.2 g, 27.7 mmol, 3.0 eq), Chloro[(diadamantan-1-yl)(n-butyl)phosphino][2-aminao-1,1-biphenyl-2-yl]palladium(II) (616 mg, 0.9 mmol, 0.1 eq). The resulting mixture was stirred for overnight at 110° C. under nitrogen atmosphere. The mixture was allowed to cool down to 25° C. The resulting mixture was diluted with water (300 The resulting mixture was extracted with Ethyl acetate (2×100 mL). The combined organic layers were washed with brine (1×300 mL), dried over anhydrous Na₂SO₄. After filtration, the filtrate was concentrated under reduced pressure. The residue/crude product was purified by reverse phase flash with the following conditions (80% CH₃CN in water (0.05% TFA)) to afford tert-butyl (2S)-2-[2-(2,2-dimethyl-5,6-dihydropyran-4-yl)phenyl]pyrrolidine-1-carboxylate (2 g, 66.9%) as a yellow solid. ¹HNMR (300 MHz, Chloroform-d) δ 7.22 (ddd, J=7.0, 4.9, 1.9 Hz, 2H), 7.15 (t, J=7.0 Hz, 1H), 7.06 (ddd, J=7.4, 3.2, 1.8 Hz, 1H), 5.58 (d, J=31.1 Hz, 1H), 4.94 (s, 1H), 4.34 (q, J=2.7 Hz, 1H), 3.96 (t, J=5.4 Hz, 1H), 3.78-3.51 (m, 2H), 2.50-2.13 (m, 5H), 1.85 (dtd, J=23.2, 15.1, 14.6, 7.0 Hz, 4H), 1.36 (d, J=2.4 Hz, 9H), 1.25-1.07 (m, 6H).

Synthesis of tert-butyl (2R)-2-{2-[(4R)-2,2-dimethyloxan-4-yl]phenyl}pyrrolidine-1-carboxylate(assumed) and tert-butyl (2R)-2-{2-[(4S)-2,2-dimethyloxan-4-yl]phenyl}pyrrolidine-1-carboxylate(assumed): Into a 100 mL round-bottom flask were added MeOH (20 mL) and tert-butyl (2R)-2-[2-(2,2-dimethyl-5,6-dihydropyran-4-yl)phenyl]pyrrolidine-1-carboxylate (500 mg, 1.4 mmol, 1.0 eq), Pd(OH)₂/C (20 mg, 0.1 mmol, 0.1 eq) and at 25° C. The resulting mixture was stirred for 12 hours at 25° C. under hydrogen (3 atm) atmosphere. The resulting mixture was filtered. the filter cake was washed with MeOH (50 mL). The crude product (450 mg) was purified by Prep-HPLC with the following conditions: Column, SunFire Prep C18 OBD Column, 19*150 mm, 5 μm 10 nm; mobile phase, water (0.05% TFA) and CH₃CN (30% CH₃CN up to 95% in 7 min); Detector, UV 220 to afford tert-butyl (2R)-2-{2-[(4R)-2,2-dimethyloxan-4-yl]phenyl}pyrrolidine-1-carboxylate(assumed) (200 mg, 39.7%) as white solid. and tert-butyl (2R)-2-{2-[(4S)-2,2-dimethyloxan-4-yl]phenyl}pyrrolidine-1-carboxylate(assumed) (200 mg, 39.7%) as white solid. 3A: LC-MS (ESI, m/z) M+1:360. ¹HNMR (300 MHz, DMSO-d₆) δ 7.29-7.11 (m, 3H), 7.01 (d, J=7.5 Hz, 1H), 5.02 (s, 1H), 3.74 (d, J=8.3 Hz, 2H), 3.60 (dt, J=10.5, 6.6 Hz, 1H), 3.53-3.40 (m, 1H), 3.16 (t, J=12.3 Hz, 1H), 2.39 (d, J=9.7 Hz, 1H), 1.84 (q, J=6.1 Hz, 2H), 1.75-1.48 (m, 4H), 1.42 (d, J=20.7 Hz, 4H), 1.29 (s, 3H), 1.16 (s, 3H), 1.08 (s, 6H). 3B: LC. MS (ESI, m/z) M+1:360. ¹HNMR (300 MHz, DMSO-d₆) δ 7.26 (d, J=7.5 Hz, 1H), 7.23-7.09 (m, 2H), 7.00 (d, J=6.8 Hz, 1H), 5.16 (d, J=22.6 Hz, 1H), 3.87-3.65 (m, 2H), 3.59 (ddd, J=10.6, 7.2, 5.1 Hz, 1H), 3.48 (dt, J=10.5, 7.5 Hz, 1H), 3.23 (s, 1H), 2.47-2.24 (m, 2H), 1.82 (q, J=6.4 Hz, 2H), 1.54 (d, J=22.4 Hz, 5H), 1.38 (d, J=7.8 Hz, 3H), 1.28 (s, 3H), 1.18 (s, 3H), 1.07 (s, 7H).

Synthesis of (2R)-2-{2-[(4R)-2,2-dimethyloxan-4-yl]phenyl}pyrrolidine Into a 50 mL round-bottom flask were added tert-butyl (2R)-2-{2-[(4R)-2,2-dimethyloxan-4-yl]phenyl}pyrrolidine-1-carboxylate (75 mg, 0.2 mmol, 1.0 eq), CH₂Cl₂ (1.5 mL) and TFA (0.5 mL) at 25° C. The resulting mixture was stirred for 3 hours at 25° C. The resulting mixture was concentrated under reduced pressure. This resulted in (2R)-2-{2-[(4R)-2,2-dimethyloxan-4-yl]phenyl}pyrrolidine (55 mg, crude) as light yellow oil. LC-MS (ESI, m/z) M+1: 260.

Synthesis of 4-{2-[(2S)-2-{2-[(4R)-2,2-dimethyloxan-4-yl]phenyl}pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide Into a 8 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.07 mmol, 1.0 eq) and (2S)-2-{2-[(4R)-2,2-dimethyloxan-4-yl]phenyl}pyrrolidine (55 mg, 0.2 mmol, 3.0 eq), CH₂Cl₂ (2 mL), NaBH(OAc)₃ (28 mg, 0.1 mmol, 2.0 eq) at 25° C. The resulting mixture was stirred for 16 hours at 25° C. The reaction was quenched by the addition of MeOH (2.0 mL) at 25° C. The resulting mixture was concentrated under reduced pressure. The residue was purified by TLC eluted with dichloromethane/methanol (95:5) to afford 4-{2-[(2S)-2-{2-[(4R)-2,2-dimethyloxan-4-yl]phenyl}pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 78.7%) as yellow solid. LC-MS (ESI, m/z) M+1:1145.

Synthesis of 4-{2-[(2S)-2-{2-[(4R)-2,2-dimethyloxan-4-yl]phenyl}pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide)(assumed): Into a 8 mL vial were added 4-{2-[(2S)-2-{2-[(4R)-2,2-dimethyloxan-4-yl]phenyl}pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.05 mmol, 1.0 eq) and ethylenediamine (47 mg, 0.8 mmol, 15 eq) at 25° C. The resulting mixture was stirred for 3 hours at 70° C. The mixture was allowed to cool down to 25° C. The resulting mixture was diluted with water (5 mL). The resulting mixture was extracted with CH₂Cl₂ (3×5 mL). The combined organic layers were washed with brine (1×5 mL), dried over anhydrous Na₂SO₄. After filtration, the filtrate was concentrated under reduced pressure. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization. This resulted in 4-{2-[(2S)-2-{2-[(4R)-2,2-dimethyloxan-4-yl]phenyl}pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide)(assumed) (11 mg, 20.6%) as yellow solid. LC-MS (ESI, m/z) M+1:1015. ¹HNMR (300 MHz, Chloroform-d) δ 12.43 (s, 1H), 8.57 (d, J=2.3 Hz, 1H), 8.48 (s, 2H), 8.07 (d, J=9.2 Hz, 1H), 7.87-7.77 (m, 1H), 7.45 (d, J=28.7 Hz, 1H), 7.23 (s, 3H), 7.16-7.08 (m, 1H), 6.84 (d, J=9.2 Hz, 1H), 6.78-6.65 (m, 2H), 6.49 (s, 1H), 6.09 (s, 1H), 5.04 (q, J=8.3 Hz, 1H), 4.50 (t, J=7.7 Hz, 1H), 4.13-4.00 (m, 4H), 3.99-3.88 (m, 1H), 3.81 (t, J=13.0 Hz, 2H), 3.44 (t, J=11.8 Hz, 2H), 3.22 (t, J=6.2 Hz, 2H), 3.10 (s, 7H), 2.63-2.12 (m, 4H), 2.05-1.89 (m, 4H), 1.85-1.69 (m, 6H), 1.51-1.36 (m, 2H), 1.31 (d, J=20.1 Hz, 13H).

### Example 33: Preparation of 4-{2-[(2S)-2-(3-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), ethyl acetate (20 mL) and 2.0 M HCl in ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N₁,N₂-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of 4-{2-[(2S)-2-(3-chlorophenyl)pyrrolidin-1-yl]-7-azaspiro[3.5]nonan-7-yl}-N-{3-nitro. 4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1.0 eq), (2S)-2-(3-chlorophenyl)pyrrolidine (12 mg, 0.1 mmol, 1.0 eq), CH₂Cl₂ (3 mL), NaBH(OAc)₃ (28 mg, 0.1 mmol, 2.0 eq). The resulting mixture was stirred for overnight at 25° C. The resulting mixture was concentrated under vacuum. The residue was purified by Prep-TLC (dichloromethane/methanol=12: 1) to afford 4-{2-[(2S)-2-(3-chlorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 84.5%) as a yellow solid. LC-MS (ESI, m/z) M+1:1067.

Synthesis of 4-{2-[(2S)-2-(3-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide: Into a 100 mL round-bottom flask were added 4-{2-[(2S)-2-(3-chlorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1.0 eq), 1.0 M TBAF in THE (5 mL), ethylenediamine (68 mg, 1.1 mmol, 20.0 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃ and 0.1% NH₃·H₂O) and CH₃CN (45% CH₃CN up to 85% in 10 min); Detector, UV. This resulted in 4-{2-[(2S)-2-(3-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide (5 mg, 9.5%) as a yellow solid. LC-MS (ESI, m/z) M+1:937. ¹HNMR (300 MHz, Chloroform-d) δ 12.45 (s, 1H), 9.17 (d, J=19.7 Hz, 1H), 8.69-8.33 (m, 2H), 8.07 (d, J=9.1 Hz, 1H), 7.86-7.66 (m, 1H), 7.43 (d, J=40.6 Hz, 2H), 7.18 (d, J=19.7 Hz, 4H), 6.85 (d, J=9.2 Hz, 1H), 6.79-6.64 (m, 2H), 6.51 (d, J=2.4 Hz, 1H), 6.09 (d, J=3.5 Hz, 1H), 5.04 (q, J=8.4 Hz, 1H), 4.49 (t, J=7.7 Hz, 1H), 4.15-3.75 (m, 6H), 3.44 (t, J=11.7 Hz, 2H), 3.14 (dtd, J=42.2, 23.3, 21.3, 13.7 Hz, 8H), 2.39-2.11 (m, 2H), 1.80 (dd, J=35.9, 25.5 Hz, 8H), 1.62-1.28 (m, 8H).

### Example 34: Preparation of 4-{2-[(2S)-2-(4-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), ethyl acetate (20 mL) and 2.0 M HCl in ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of 4-{2-[(2S)-2-(4-chlorophenyl)pyrrolidin-1-yl]-7-azaspiro[3.5]nonan-7-yl}-N-{3-nitro. 4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1.0 eq), (2S)-2-(4-chlorophenyl)pyrrolidine (12 mg, 0.1 mmol, 1.0 eq), CH₂Cl₂ (3 mL), NaBH(OAc)₃ (28 mg, 0.1 mmol, 2.0 eq). The resulting mixture was stirred for overnight at 25° C. The resulting mixture was concentrated under vacuum. The residue was purified by Prep-TLC (dichloromethane/methanol=12: 1) to afford 4-{2-[(2S)-2-(4-chlorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 84.5%) as a yellow solid. LC-MS (ESI, m/z) M+1:1067.

Synthesis of 4-{2-[(2S)-2-(4-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide: Into a 100 mL round-bottom flask were added 4-{2-[(2S)-2-(4-chlorophenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (60 mg, 0.1 mmol, 1 eq), 1.0 M TBAF in THE (5 mL), ethylenediamine (68 mg, 1.1 mmol, 20.0 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃ and 0.1% NH₃·H₂O) and CH₃CN (45% CH₃CN up to 85% in 10 min); Detector, UV. This resulted in 4-{2-[(2S)-2-(4-chlorophenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide (10 mg, 18.9%) as a yellow solid. LC-MS (ESI, m/z) M+1:937. ¹HNMR (300 MHz, Chloroform-d) δ 12.44 (s, 1H), 8.61-8.41 (m, 4H), 8.08 (d, J=9.2 Hz, 1H), 7.82 (dd, J=9.1, 2.3 Hz, 1H), 7.27 (d, J=3.0 Hz, 4H), 7.16-7.07 (m, 1H), 6.85 (d, J=10.1 Hz, 1H), 6.78-6.65 (m, 2H), 6.50 (d, J=2.4 Hz, 1H), 6.41-6.06 (m, 2H), 5.04 (q, J=8.5 Hz, 1H), 4.50 (t, J=7.7 Hz, 1H), 4.19-3.72 (m, 7H), 3.44 (t, J=11.8 Hz, 2H), 3.35-2.85 (m, 6H), 2.37-1.20 (m, 18H).

### Example 35: Preparation of 4-(2-((S)-2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2.0 M HCl in Ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹HNMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1: 2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352. ¹HNMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4 eq) at 25° C. The resulting mixture was stirred for 2 hours at 40° C. The mixture was allowed to cool down to 25° C. The resulting mixture was diluted with water (20 mL). The mixture was acidified to pH=6 with 1 M HCl. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338. ¹HNMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCI (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635. ¹HNMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

Synthesis of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.2 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/dichloromethane=10: 1) to afford N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (400 mg, 47.0%) as yellow solid. LC-MS (ESI, m/z) M+1: 902. ¹HNMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dq, J=16.8, 5.4, 4.9 Hz, 3H), 0.91-0.76 (m, 3H), −0.06 (s, 9H).

Synthesis of tert-butyl (2S)-2-(2-bromophenyl)pyrrolidine-1-carboxylate: Into a 250-mL round-bottom flask, were placed (2S)-2-(2-bromophenyl)pyrrolidine (17.0 g, 75.2 mmol, 1.0 eq), CH₂Cl₂ (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1:15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (S)-2-(2-isobutylphenyl)pyrrolidine-1-carboxylate: Into a 40-mL vial were added tert-butyl (2S)-2-(2-bromophenyl)pyrrolidine-1-carboxylate (600 mg, 1.8 mmol, 1.0 eq), THE (12 mL), Ad₂nBu Pd G2 (123 mg, 0.2 mmol, 0.1 eq), tert-butyl(chloro)magnesium (2 mL, 3.7 mmol, 2.0 eq) The resulting mixture was stirred for overnight at 25° C. under nitrogen atmosphere. The resulting mixture was diluted with water (20 mL). The resulting mixture was extracted with Ethyl acetate (2×50 mL). The combined organic layers were washed with brine (1×50 mL), dried over anhydrous Na₂SO₄. After filtration, the filtrate was concentrated under reduced pressure. The residue was purified by reverse flash chromatography with the following conditions: column, C18 silica gel; mobile phase, CN₃CN in water (0.05% TFA), 30% to 90% gradient in 10 min; detector, UV 220 nm. This resulted in tert-butyl (S)-2-(2-isobutylphenyl)pyrrolidine-1-carboxylate (120 mg, 21.5%) as a white solid. LC-MS (ESI, m/z) M+1: 304.

Synthesis of (S)-2-(2-isobutylphenyl)pyrrolidine hydrochloride: Into a 8-mL vial were added tert-butyl (S)-2-(2-isobutylphenyl)pyrrolidine-1-carboxylate (120 mg, 0.4 mmol, 1.0 eq) and HCl(gas) in 1,4-dioxane (4N, 2 mL) at 25° C. The resulting mixture was stirred for 2 hours at 25° C. The resulting mixture was concentrated under reduced pressure. The residue was purified by trituration with ethyl ether (5 mL). The precipitated solids were collected by filtration and washed with ethyl ether (2×5 mL). This resulted in (S)-2-(2-isobutylphenyl)pyrrolidine hydrochloride (90 mg, 94.9%) as a white solid. LC-MS (ESI, m/z) M+1: 204. ¹H NMR (400 MHz, Chloroform-d) δ 10.28 (s, 1H), 9.15 (s, 1H), 7.69 (d, J=7.6 Hz, 1H), 7.35-7.20 (m, 2H), 7.12 (dd, J=7.5, 1.5 Hz, 1H), 4.71 (t, J=8.4 Hz, 1H), 3.43 (s, 1H), 3.16 (s, 1H), 2.80 (dd, J=14.1, 6.8 Hz, 1H), 2.42 (dd, J=14.1, 7.9 Hz, 1H), 2.35-2.24 (m, 1H), 2.15 (s, 1H), 2.06-1.96 (m, 2H), 1.77 (hept, J=6.7 Hz, 1H), 0.91 (dd, J=16.1, 6.5 Hz, 6H).

Synthesis of 4-(2-((S)-2-(2-isobutylphenyl)pyrrolidin-1-yl)-7-azaspiro[3.5]nonan-7-yl)-N-((3-nitro. 4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin. 5(1H)-yl)benzamide: Into a 8-mL vial were added N-((3-nitro-4-(((tetrahydro-2H-pyran-4-yl)methyl)amino)phenyl)sulfonyl)-4-(2-oxo-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (45 mg, 0.1 mmol, 1.0 eq), (S)-2-(2-isobutylphenyl)pyrrolidine hydrochloride (36 mg, 0.2 mmol, 3.0 eq), CH₂Cl₂ (2 mL), NaBH(OAc)₃ (63 mg, 0.3 mmol, 6.0 eq) at 25° C. The resulting mixture was stirred for overnight at 25° C. under nitrogen atmosphere. The resulting mixture was diluted with water (10 mL). The resulting mixture was extracted with CH₂Cl₂ (2×20 mL). The combined organic layers were washed with brine (1×20 mL), dried over anhydrous Na₂SO₄. After filtration, the filtrate was concentrated under reduced pressure. The residue was purified by Prep-TLC (CH₂Cl₂/MeOH=13: 1) to afford 4-(2-((S)-2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (50 mg, 90.5%) as a yellow solid. LC-MS (ESI, m/z) M+1: 1090.

Synthesis of 4-(2-((S)-2-(2-isobutylphenyl)pyrrolidin-1-yl)-7-azaspiro[3.5]nonan-7-yl)-N-((3-nitro. 4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide: Into a 8-mL vial were added 4-(2-((S)-2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (50 mg, 0.1 mmol, 1.0 eq), TBAF (1.0 mL, 1 M in THF) and ethylenediamine (41 mg, 0.7 mmol, 15.0 eq) at 25° C. The resulting mixture was stirred for 4 hours at 70° C. The mixture was allowed to cool down to 25° C. The resulting mixture was diluted with water (10 mL). The resulting mixture was extracted with Ethyl acetate (2×10 mL). The combined organic layers were washed with brine (1×10 mL), dried over anhydrous Na₂SO₄. After filtration, the filtrate was concentrated under reduced pressure. The crude product was purified by Prep-HPLC with the following conditions (Prep-HPLC-006): Column, Sunfire Prep C18 OBD, 50*250 mm, 5 μm; mobile phase, water (0.05% TFA) and CH₃CN (20% CH₃CN up to 55% in 12 min); Detector, UV, 220 nm. This resulted in 4-(2-((S)-2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide (13 mg, 29.5%) as a yellow solid. LC-MS (ESI, m/z) M+1: 959. ¹H NMR (400 MHz, DMSO-d₆) δ 12.30 (s, 1H), 11.23 (d, J=2.3 Hz, 1H), 9.61 (s, 1H), 8.55 (t, J=5.9 Hz, 1H), 8.32 (d, J=2.3 Hz, 1H), 7.74-7.59 (m, 2H), 7.52-7.39 (m, 1H), 7.33 (dt, J=5.5, 2.1 Hz, 2H), 7.24-7.16 (m, 1H), 7.15-7.02 (m, 1H), 6.93-6.83 (m, 1H), 6.82 (d, J=2.4 Hz, 1H), 6.80-6.71 (m, 1H), 6.58 (s, 1H), 6.01 (ddd, J=13.8, 3.4, 1.9 Hz, 1H), 4.65 (s, 1H), 4.65-4.55 (m, 1H), 4.37-4.25 (m, 2H), 3.98-3.79 (m, 4H), 3.53-3.51 (m, 2H), 3.68 (s, 1H), 3.28 (d, J=7.6 Hz, 2H), 3.21-3.16 (m, 4H), 3.10 (s, 2H), 2.73 (dd, J=13.6, 6.9 Hz, 1H), 2.41 (h, J=6.3, 5.5 Hz, 2H), 2.22-2.06 (m, 4H), 2.01 (dq, J=9.2, 5.3, 3.5 Hz, 1H), 1.89 (dtt, J=12.4, 8.9, 4.2 Hz, 1H), 1.75-1.62 (m, 2H), 1.64-1.59 (m, 1H), 1.53-1.44 (m, 3H), 1.39 (q, J=7.4, 6.8 Hz, 1H), 1.35-1.16 (m, 4H), 0.87 (dt, J=18.8, 5.6 Hz, 6H).

### Example 36: Preparation of 4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide hydrochloride

Synthesis of methyl 2-bromo-4-{2-[(2S)-2-(2-isopropylphenyl)pyrrolidin-1-yl]-7-azaspiro[3.5]nonan-7-yl}benzoate: Into a 100 mL round-bottom flask were added (2S)-2-(2-isopropylphenyl)pyrrolidine hydrochloride (2.0 g, 8.8 mmol, 1.1 eq) and methyl 2-bromo-4-{2-oxospiro[3.5]nonan-7-yl}benzoate (2.8 g, 7.9 mmol, 1.0 eq), CH₂Cl₂ (30 mL), NaBH(OAc)₃ (3.4 g, 16.0 mmol, 2.0 eq). The resulting mixture was stirred for overnight at 25° C. The resulting mixture was diluted with aqueous Na₂CO₃ (200 mL). The resulting mixture was extracted with dichloromethane (3×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1:3) to afford methyl 2-bromo-4-{2-[(2S)-2-(2-isopropylphenyl)pyrrolidin-1-yl]-7-azaspiro[3.5]nonan-7-yl}benzoate (3 g, 74.0%) as a yellow oil. LC-MS (ESI, m/z) M+1: 525/527.

Synthesis of methyl 4-{2-[(2S)-2-(2-isopropylphenyl)pyrrolidin-1-yl]-7-azaspiro[3.5]nonan-7-yl}. 2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate: Into a 100 mL round-bottom flask were added methyl 2-bromo-4-{2-[(2S)-2-(2-isopropylphenyl)pyrrolidin-1-yl]-7-azaspiro[3.5]nonan-7-yl}benzoate (2.0 g, 3.8 mmol, 1.5 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (883 mg, 2.5 mmol, 1.0 eq), toluene (20 mL), Cs₂CO₃ (2.5 g, 7.7 mmol, 3.0 eq), Xantphos (589 mg, 1.0 mmol, 0.4 eq), Pd₂(dba)₃·CHCl₃ (525 mg, 0.5 mmol, 0.2 eq). The resulting mixture was stirred for overnight at 110° C. under nitrogen atmosphere. The mixture was allowed to cool down to 25° C. The resulting mixture was concentrated under vacuum. The crude residue was purified by a flash column (silica gel, ethyl acetate/petroleum ether=1:1) to afford methyl 4-{2-[(2S)-2-(2-isopropylphenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (1 g, 54.6%) as a yellow solid. LC-MS (ESI, m/z) M+1: 792.

Synthesis of methyl 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropylphenyl)pyrrolidin-1-yl]-7-azaspiro[3.5]nonan-7-yl}benzoate: Into a 100 mL round-bottom flask were added methyl 4-{2-[(2S)-2-(2-isopropylphenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (1.1 g, 1.4 mmol, 1.0 eq), TBAF in THE (1.0 M, 20 mL), ethylenediamine (1.7 g, 28.3 mmol, 20.3 eq). The resulting mixture was stirred for 5 hours at 70° C. The mixture was allowed to cool down to 25° C. The resulting mixture was diluted with water (300 mL). The resulting mixture was extracted with Ethyl acetate (3×100 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate) to afford methyl 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropylphenyl)pyrrolidin-1-yl]-7-azaspiro[3.5]nonan-7-yl}benzoate (620 mg, 67.4%) as a light yellow solid. LC. MS (ESI, m/z) M+1: 662.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropylphenyl)pyrrolidin-1-yl]-7-azaspiro[3.5]nonan-7-yl}benzoic acid: Into a 40 mL vial were added methyl 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropylphenyl)pyrrolidin-1-yl]-7-azaspiro[3.5]nonan-7-yl}benzoate (620 mg, 0.9 mmol, 1.0 eq), MeOH (5 mL), dioxane (5 mL), NaOH (4.0 M, 1 mL). The resulting mixture was stirred for 4 hours at 70° C. The mixture was allowed to cool down to 25° C. The resulting mixture was concentrated under vacuum. The mixture was acidified to pH=5 with HCl (1.0 M). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude residue was purified by a flash column (silica gel, dichloromethane/methanol=10:1) to afford 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropylphenyl)pyrrolidin-1-yl]-7-azaspiro[3.5]nonan-7-yl}benzoic acid (174 mg, 28.6%) as a light yellow solid. LC-MS (ESI, m/z) M+1: 648.

Synthesis of 4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide hydrochloride: Into a 20 mL vial were added 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropylphenyl)pyrrolidin-1-yl]-7-azaspiro[3.5]nonan-7-yl}benzoic acid (35 mg, 0.05 mmol, 1.0 eq) and 3-nitro-4-({[(1r,4r)-4-methoxy-4-methylcyclohexyl]methyl}amino)benzenesulfonamide (20 mg, 0.05 mmol, 1.0 eq), CH₂Cl₂ (3 mL), EDCI (21 mg, 0.1 mmol, 2.0 eq), DMAP (27 mg, 0.2 mmol, 4.0 eq). The resulting mixture was stirred for overnight at 30° C.

The residue was purified by Prep-TLC (dichloromethane/methanol=11:1). The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide hydrochloride (22 mg, 39.7%) as a yellow solid. LC-MS (ESI, m/z) M-HCl+1: 987. ¹HNMR (300 MHz, DMSO-d₆) δ 12.30 (s, 1H), 11.08 (d, J=124.8 Hz, 2H), 8.54 (t, J=5.9 Hz, 1H), 8.31 (d, J=2.3 Hz, 1H), 8.02 (s, 1H), 7.63 (d, J=8.9 Hz, 1H), 7.51-6.98 (m, 6H), 6.96-6.52 (m, 4H), 6.05-5.95 (m, 1H), 4.76 (d, J=8.5 Hz, 1H), 4.61 (q, J=8.4 Hz, 1H), 4.33 (t, J=7.8 Hz, 2H), 3.87 (dt, J=30.7, 8.1 Hz, 4H), 3.54 (dd, J=10.3, 7.9 Hz, 2H), 3.44-2.99 (m, 12H), 2.41 (s, 1H), 2.34-1.94 (m, 6H), 1.78 (d, J=9.6 Hz, 2H), 1.56-0.99 (m, 18H).

### Example 37: Preparation of 4-(2-((S)-2-(2-isopropylphenyl)pyrrolidin-1-yl)-7-azaspiro[3.5]nonan-7-yl)-N-((4-((((1s,4s)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide hydrochloride

Synthesis of 4-(2-((S)-2-(2-isopropylphenyl)pyrrolidin-1-yl)-7-azaspiro[3.5]nonan-7-yl)-N-((4-((((1s,4s)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide hydrochloride: Into a 20 mL vial were added 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropylphenyl)pyrrolidin-1-yl]-7-azaspiro[3.5]nonan-7-yl}benzoic acid (35 mg, 0.05 mmol, 1.0 eq) and 3-nitro-4-({[(1s,4s)-4-methoxy-4-methylcyclohexyl]methyl}amino)benzenesulfonamide (20 mg, 0.05 mmol, 1.0 eq), CH₂Cl₂ (3 mL), EDCI (21 mg, 0.1 mmol, 2.0 eq), DMAP (27 mg, 0.2 mmol, 4.0 eq). The resulting mixture was stirred for overnight at 30° C. The residue was purified by Prep-TLC (dichloromethane/methanol=11:1). The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). This resulted in 4-(2-((S)-2-(2-isopropylphenyl)pyrrolidin-1-yl)-7-azaspiro[3.5]nonan-7-yl)-N-((4-((((1s,4s)-4-methoxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide hydrochloride (21 mg, 37.9%) as a yellow solid. LC-MS (ESI, m/z) M+1: 987. ¹HNMR (300 MHz, DMSO-d₆) δ 12.30 (s, 1H), 11.08 (d, J=124.8 Hz, 2H), 8.54 (t, J=5.9 Hz, 1H), 8.31 (d, J=2.3 Hz, 1H), 8.02 (s, 1H), 7.63 (d, J=8.9 Hz, 1H), 7.51-6.98 (m, 6H), 6.96-6.52 (m, 4H), 6.05-5.95 (m, 1H), 4.76 (d, J=8.5 Hz, 1H), 4.61 (q, J=8.4 Hz, 1H), 4.33 (t, J=7.8 Hz, 2H), 3.87 (dt, J=30.7, 8.1 Hz, 4H), 3.54 (dd, J=10.3, 7.9 Hz, 2H), 3.44-2.99 (m, 12H), 2.41 (s, 1H), 2.34-1.94 (m, 6H), 1.78 (d, J=9.6 Hz, 2H), 1.56-0.99 (m, 18H).

### Example 38: Preparation of N-((4-(((4-fluorotetrahydro-2H-pyran-4-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide

Synthesis of methyl 2-bromo-4-{2-[(2S)-2-(2-methylphenyl)pyrrolidin-1-yl]-7-azaspiro[3.5]nonan. 7-yl}benzoate: Into a 100 mL round-bottom flask were added (2S)-2-(2-methylphenyl)pyrrolidine hydrochloride (1.3 g, 6.6 mmol, 1.0 eq), dichloromethane (40 mL), methyl 2-bromo-4-{2-oxo-7-azaspiro[3.5]nonan-7-yl}benzoate (2.3 g, 6.6 mmol, 1.0 eq) and NaBH(OAc)₃ (2.8 g, 13.2 mmol, 2.0 eq) at 25° C. The resulting mixture was stirred for overnight at 25° C. The resulting mixture was diluted with water (50 mL). The resulting mixture was extracted with dichloromethane (2×50 mL). The combined organic layers were washed with brine (1×50 mL), dried over anhydrous Na₂SO₄. After filtration, the filtrate was concentrated under reduced pressure. The crude residue was purified by a flash column (silica gel, ethyl acetate/petroleum ether=1:1) to afford methyl 2-bromo-4-{2-[(2S)-2-(2-methylphenyl)pyrrolidin-1-yl]-7-azaspiro[3.5]nonan-7-yl}benzoate (2.2 g, 67.3%) as a colorless oil. LC-MS (ESI, m/z) M+1:497/499.

Synthesis of methyl 4-{2-[(2S)-2-(2-methylphenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate: To a stirred mixture of methyl 2-bromo-4-{2-[(2S)-2-(2-methylphenyl)pyrrolidin-1-yl]-7-azaspiro[3.5]nonan-7-yl}benzoate (1.1 g, 2.2 mmol, 1.5 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (500 mg, 1.4 mmol, 1.0 eq) in toluene (20 mL) was added Pd₂(dba)₃·CHCl₃ (148.9 mg, 0.14 mmol, 0.1 eq), XantPhos (166.5 mg, 0.28 mmol, 0.2 eq), Cs₂CO₃ (1406.5 mg, 4.3 mmol, 3.0 eq) at 25° C. under nitrogen atmosphere. The resulting mixture was stirred for overnight at 100° C. under nitrogen atmosphere. The mixture was allowed to cool down to 25° C. The resulting mixture was diluted with water (20 mL). The resulting mixture was extracted with Ethyl acetate (2×50 mL). The combined organic layers were washed with brine (1×50 mL), dried over anhydrous Na₂SO₄. After filtration, the filtrate was concentrated under reduced pressure. The crude residue was purified by a flash column (silica gel, ethyl acetate/petroleum ether=1:1) to afford methyl 4-{2-[(2S)-2-(2-methylphenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (600 mg, 54.58%) as a colorless oil. LC-MS (ESI, m/z) M+1: 764.

Synthesis of methyl 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}benzoate: Into a 8 mL vial were added methyl 4-{2-[(2S)-2-(2-methylphenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (500 mg, 0.7 mmol, 1 eq), ethylenediamine (786.6 mg, 13.1 mmol, 20.0 eq) and TBAF (3 mL, 11.5 mmol, 17.5 eq) at 25° C. The resulting mixture was stirred for overnight at 70° C. The mixture was allowed to cool down to 25° C. and diluted with water (5 mL). The resulting mixture was extracted with Ethyl acetate (2×10 mL). The combined organic layers were washed with brine (1×20 mL), dried over anhydrous Na₂SO₄. After filtration, the filtrate was concentrated under reduced pressure. The crude residue was purified by a flash column (silica gel, CH₂Cl₂/CH₃OH=10:1) to afford methyl 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}benzoate (300 mg, 72.3%) as a yellow oil. LC-MS (ESI, m/z) M+1: 634.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}benzoic acid: Into a 25 mL round-bottom flask were added methyl 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}benzoate (300 mg, 0.5 mmol, 1.0 eq), CH₃OH (2 mL), dioxane (2 mL) and NaOH (0.4 mL, 4.0 M) at 25° C. The resulting mixture was stirred for 4 hours at 70° C. The mixture was allowed to cool down to 70° C. The resulting mixture was diluted with water (10 mL). The mixture was acidified to pH=6 with HCl (1.0 M). The resulting mixture was extracted with Ethyl acetate (2×10 mL). The combined organic layers were washed with brine (1×20 mL), dried over anhydrous Na₂SO₄. After filtration, the filtrate was concentrated under reduced pressure. The crude residue was purified by a flash column (silica gel, CH₂Cl₂/CH₃OH=10:1) to afford 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}benzoic acid (100 mg, 34.1%) as a yellow oil. LC-MS (ESI, m/z) M+1: 620.

Synthesis of N-((4-(((4-fluorotetrahydro-2H-pyran-4-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide: A solution of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}benzoic acid (25 mg, 0.04 mmol, 1.0 eq), 4-{[(4-fluorooxan-4-yl)methyl]amino}-3-nitrobenzenesulfonamide (13 mg, 0.04 mmol, 1.0 eq), EDCI (16 mg, 0.08 mmol, 2.0 eq) and N,N-dimethylpyridin-4-amine (20 mg, 0.16 mmol, 4.0 eq) in CH₂Cl₂ (3 mL) was stirred for overnight at 30° C. The resulting mixture was diluted with water (10 mL). The resulting mixture was extracted with Ethyl acetate (2×10 mL). The combined organic layers were washed with brine (1×10 mL), dried over anhydrous Na₂SO₄. After filtration, the filtrate was concentrated under reduced pressure. The residue was purified by Prep-HPLC with the following conditions (Prep-HPLC-006): Column, YMC-Actus Triart C18 ExRS, 30*150 mm, 5 μm; mobile phase, water (0.05% NH₃·H₂O) and CH₃CN (45% CH₃CN up to 85% in 10 min) to afford N-((4-(((4-fluorotetrahydro-2H-pyran-4-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide (6 mg, 15.9%) as a yellow solid. LC-MS (ESI, m/z) M+1: 935. ¹HNMR (300 MHz, DMSO-d₆) δ 11.12 (s, 1H), 10.98 (s, 1H), 8.42 (s, 1H), 8.32 (dd, J=18.7, 2.2 Hz, 1H), 7.51 (d, J=7.3 Hz, 2H), 7.35 (s, 1H), 7.10-7.02 (m, 4H), 6.82-6.63 (m, 3H), 6.54 (s, 1H), 6.06-5.93 (m, 1H), 4.69-4.57 (m, 1H), 4.45 (d, J=8.2 Hz, 1H), 4.34-3.98 (m, 3H), 3.89-3.71 (m, 6H), 3.21-2.96 (m, 8H), 2.30 (d, J=4.1 Hz, 4H), 1.90-1.69 (m, 8H), 1.58-1.40 (m, 7H), 1.23 (s, 1H).

### Example 39: Preparation of N-((4-((((S)-1,4-dioxan-2-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide

Synthesis of N-((4-((((S)-1,4-dioxan-2-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o. tolyl)pyrrolidin-1-yl)-7-azaspiro[3.5]nonan-7-yl)benzamide: A solution of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}benzoic acid (25 mg, 0.04 mmol, 1.0 eq), 4-{[(2S)-1,4-dioxan-2-ylmethyl]amino}-3-nitrobenzenesulfonamide (13 mg, 0.04 mmol, 1.0 eq), EDCI (16 mg, 0.08 mmol, 2.0 eq) and DMAP (20 mg, 0.16 mmol, 4.0 eq) in CH₂Cl₂ (3 mL) was stirred for overnight at 30° C. The resulting mixture was diluted with water (10 mL). The resulting mixture was extracted with Ethyl acetate (2×10 mL). The combined organic layers were washed with brine (1×10 mL), dried over anhydrous Na₂SO₄. After filtration, the filtrate was concentrated under reduced pressure. The residue was purified by Prep-HPLC with the following conditions (Prep-HPLC-006): Column, YMC-Actus Triart C18 ExRS, 30*150 mm, 5 μm; mobile phase, water (0.05% NH₃·H₂O) and CH₃CN (45% CH₃CN up to 85% in 10 min) to afford N-((4-((((S)-1,4-dioxan-2-yl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide (5 mg, 13.5%) as a yellow solid. LC-MS (ESI, m/z) M+1: 919. ¹HNMR (300 MHz, DMSO-d₆) δ 11.05 (s, 1H), 10.91 (s, 1H), 8.36-8.25 (m, 5H), 7.50 (d, J=7.5 Hz, 4H), 7.10 (s, 8H), 7.08-7.02 (m, 4H), 6.77 (s, 1H), 6.71 (s, 3H), 6.68 (s, 2H), 6.61 (d, J=2.4 Hz, 2H), 6.52 (s, 2H), 6.05-5.92 (m, 1H), 2.69 (s, 2H), 2.30 (d, J=3.3 Hz, 4H), 1.83 (s, 5H), 1.56-1.46 (m, 8H), 1.23 (s, 1H).

### Example 40: Preparation of N-((4-((((1s,4s)-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide

Synthesis of N-((4-((((1s,4s)-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide: A solution of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}benzoic acid (25 mg, 0.04 mmol, 1.0 eq), 3-nitro-4-({[(1s,4s)-4-hydroxy-4-methylcyclohexyl]methyl}amino)benzenesulfonamide (14 mg, 0.04 mmol, 1.0 eq), EDCI (16 mg, 0.08 mmol, 2.0 eq) and DMAP (20 mg, 0.16 mmol, 4.0 eq) in CH₂Cl₂ (3 mL) was stirred for overnight at 30° C. The resulting mixture was diluted with water (10 mL). The resulting mixture was extracted with Ethyl acetate (2×10 mL). The combined organic layers were washed with brine (1×10 mL), dried over anhydrous Na₂SO₄. After filtration, the filtrate was concentrated under reduced pressure. The residue was purified by Prep-HPLC with the following conditions (Prep-HPLC-006): Column, YMC-Actus Triart C18 ExRS, 30*150 mm, 5 μm; mobile phase, water (0.05% NH₃·H₂O) and CH₃CN (45% CH₃CN up to 85% in 10 min) to afford N-((4-((((1s,4s)-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide (10 mg, 26.2%) as a yellow solid. LC-MS (ESI, m/z) M+1: 945. ¹HNMR (300 MHz, DMSO-d₆) δ 11.15 (s, 1H), 10.97 (s, 1H), 8.59-8.04 (m, 2H), 7.75-7.39 (m, 3H), 7.39-6.98 (m, 5H), 6.69 (dd, J=63.0, 27.3 Hz, 4H), 4.90-3.74 (m, 9H), 3.30-2.84 (m, 6H), 2.30 (d, J=4.2 Hz, 5H), 1.80 (d, J=24.5 Hz, 5H), 1.65-1.35 (m, 18H), 1.25-1.10 (m, 3H).

### Example 41: Preparation of N-((4-((((1r,4r)-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide

Synthesis of N-((4-((((1r,4r)-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide: A solution of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-methylphenyl)pyrrolidin-1-yl]-7-azaspiro[3.5]nonan-7-yl}benzoic acid (25 mg, 0.04 mmol, 1.0 eq), 3-nitro-4-({[(1r,4r)-4-hydroxy-4-methylcyclohexyl]methyl}amino)benzenesulfonamide (14 mg, 0.04 mmol, 1.0 eq), EDCI (16 mg, 0.08 mmol, 2.0 eq) and DMAP (20 mg, 0.16 mmol, 4.0 eq) in CH₂Cl₂ (3 mL) was stirred for overnight at 30° C. The resulting mixture was diluted with water (10 mL). The resulting mixture was extracted with Ethyl acetate (2×10 mL). The combined organic layers were washed with brine (1×10 mL), dried over anhydrous Na₂SO₄. After filtration, the filtrate was concentrated under reduced pressure. The residue was purified by Prep-HPLC with the following conditions (Prep-HPLC-006): Column, YMC-Actus Triart C18 ExRS, 30*150 mm, 5 μm; mobile phase, water (0.05% NH₃·H₂O) and CH₃CN (45% CH₃CN up to 85% in 10 min) to afford N-((4-((((1r,4r)-4-hydroxy-4-methylcyclohexyl)methyl)amino)-3-nitrophenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-2-(o-tolyl)pyrrolidin-1-yl)-7-azaspiro[3.5]nonan-7-yl)benzamide (11 mg, 28.9%) as a yellow solid. LC-MS (ESI, m/z) M+1: 945. ¹HNMR (300 MHz, DMSO-d₆) δ 11.10 (s, 1H), 10.96 (s, 1H), 8.30 (dd, J=19.1, 2.1 Hz, 2H), 7.54 (dd, J=19.9, 8.1 Hz, 2H), 7.20-7.03 (m, 5H), 6.91-6.43 (m, 4H), 6.00 (ddd, J=17.8, 3.3, 1.8 Hz, 1H), 4.90-3.74 (m, 10H), 3.30-2.84 (m, 5H), 2.30 (d, J=4.2 Hz, 1H), 1.84-1.35 (m, 19H), 1.25-1.10 (m, 6H).

### Example 42: Preparation of N-((4-(((4-fluorotetrahydro-2H-pyran-4-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide

Synthesis of methyl 2-bromo-4-{2-[(2S)-2-(2-isopropoxyphenyl)pyrrolidin-1-yl]-7-azaspiro[3.5]nonan-7-yl}benzoate: Into a 100 mL round-bottom flask were added (2S)-2-(2-isopropoxyphenyl)pyrrolidine (770 mg, 3.7 mmol, 1.0 eq), CH₂Cl₂ (30 mL), methyl 2-bromo-4-{2-oxo-7-azaspiro[3.5]nonan-7-yl}benzoate (1.3 g, 3.7 mmol, 1.0 eq), NaBH(OAc)₃ (1.6 g, 7.5 mmol, 2.0 eq). The resulting mixture was stirred for overnight at 25° C. The resulting mixture was concentrated under reduced pressure. The crude residue was purified by a flash column (silica gel, ethyl acetate/petroleum ether=1:1) to afford methyl 2-bromo-4-{2-[(2S)-2-(2-isopropoxyphenyl)pyrrolidin-1-yl]-7-azaspiro[3.5]nonan-7-yl}benzoate (926 mg, 45.6%) as a yellow oil. LC-MS (ESI, m/z) M+1: 541/543. ¹HNMR (300 MHz, Chloroform-d) δ 7.80 (d, J=8.9 Hz, 1H), 7.58 (d, J=7.7 Hz, 1H), 7.17 (t, J=7.6 Hz, 1H), 7.06 (d, J=2.5 Hz, 1H), 6.94 (t, J=7.4 Hz, 1H), 6.85 (d, J=8.2 Hz, 1H), 6.74 (dd, J=9.0, 2.6 Hz, 1H), 4.56 (p, J=6.1 Hz, 1H), 3.91 (d, J=8.2 Hz, 1H), 3.87 (s, 3H), 3.28-3.07 (m, 6H), 2.52-2.33 (m, 1H), 2.21 (p, J=7.9 Hz, 1H), 1.93-1.52 (m, 11H), 1.36 (dd, J=6.1, 3.5 Hz, 6H).

Synthesis of methyl 4-{2-[(2S)-2-(2-isopropoxyphenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate: Into a 40 mL vial were added methyl 2-bromo-4-{2-[(2S)-2-(2-isopropoxyphenyl)pyrrolidin-1-yl]-7-azaspiro[3.5]nonan-7-yl}benzoate (920 mg, 1.7 mmol, 1.5 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (395 mg, 1.1 mmol, 1.0 eq), toluene (10 mL), Cs₂CO₃ (1.1 g, 3.4 mmol, 3.0 eq), Pd₂(dba)₃·CHCl₃ (118 mg, 0.1 mmol, 0.1 eq), Xantphos (132 mg, 0.2 mmol, 0.2 eq). The resulting mixture was stirred for 8 hours at 110° C. under nitrogen atmosphere. The mixture was allowed to cool down to 25° C. The resulting mixture was concentrated under vacuum. The crude residue was purified by a flash column (silica gel, ethyl acetate) to afford methyl 4-{2-[(2S)-2-(2-isopropoxyphenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (580 mg, 63.1%) as a yellow solid. LC-MS (ESI, m/z) M+1: 808.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropoxyphenyl)pyrrolidin-1-yl]-7-azaspiro[3.5]nonan-7-yl}benzoic acid: Into a 100 mL round-bottom flask were added methyl 4-{2-[(2S)-2-(2-isopropoxyphenyl)pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzoate (570 mg, 0.7 mmol, 1.0 eq), TBAF in THE (1.0 M, 10 mL), ethylenediamine (850 mg, 14.1 mmol, 20.0 eq). The resulting mixture was stirred for overnight at 70° C. The mixture was allowed to cool down to 25° C. The resulting mixture was diluted with water (200 mL). The resulting mixture was extracted with Ethyl acetate (3×50 mL). The combined organic layers were washed with brine (1×200 mL), dried over anhydrous Na₂SO₄. After filtration, the filtrate was concentrated under reduced pressure. The crude residue was purified by a flash column (silica gel, dichloromethane/methanol=10:1) to afford 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropoxyphenyl)pyrrolidin-1-yl]-7-azaspiro[3.5]nonan-7-yl}benzoic acid (154 mg, 32.8%) as a yellow solid. LC-MS (ESI, m/z) M+1: 664.

Synthesis of N-((4-(((4-fluorotetrahydro-2H-pyran-4-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide: Into a 40 mL vial were added 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropoxyphenyl)pyrrolidin-1-yl]-7-azaspiro[3.5]nonan-7-yl}benzoic acid (30 mg, 0.05 mmol, 1.0 eq) and 4-{[(4-fluorooxan-4-yl)methyl]amino}-3-nitrobenzenesulfonamide (15 mg, 0.05 mmol, 1.0 eq), CH₂Cl₂ (3 mL), EDCI (17 mg, 0.09 mmol, 2.0 eq), DMAP (22 mg, 0.18 mmol, 4.0 eq). The resulting mixture was stirred for overnight at 25° C. The resulting mixture was concentrated under vacuum. The residue was purified by Prep-TLC (dichloromethane/methanol=12:1). The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in N-((4-(((4-fluorotetrahydro-2H-pyran-4-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide (9 mg, 20.3%) as a yellow solid. LC-MS (ESI, m/z) M+1: 979. ¹HNMR (300 MHz, DMSO-d₆) δ 11.04 (d, J=44.6 Hz, 1H), 8.48-8.25 (m, 2H), 7.53 (dd, J=45.2, 8.3 Hz, 2H), 7.28 (s, 2H), 7.12-6.48 (m, 7H), 6.00 (dt, J=22.0, 2.6 Hz, 1H), 4.75-4.54 (m, 2H), 4.51-4.00 (m, 3H), 3.90-3.63 (m, 7H), 3.21-2.91 (m, 6H), 2.30-1.66 (m, 11H), 1.46 (d, J=22.9 Hz, 6H), 1.30 (dt, J=9.8, 5.2 Hz, 6H).

### Example 43: Preparation of N-((4-((((S)-1,4-dioxan-2-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide

Synthesis of N-((4-((((S)-1,4-dioxan-2-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide: Into a 40 mL vial were added 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropoxyphenyl)pyrrolidin-1-yl]-7-azaspiro[3.5]nonan-7-yl}benzoic acid (30 mg, 0.05 mmol, 1.0 eq) and 4-{[(2S)-1,4-dioxan-2-ylmethyl]amino}-3-nitrobenzenesulfonamide (14 mg, 0.05 mmol, 1.0 eq), CH₂Cl₂ (3 mL), EDCI (17 mg, 0.1 mmol, 2.0 eq), DMAP (22 mg, 0.2 mmol, 4.0 eq). The resulting mixture was stirred for overnight at 25° C. The resulting mixture was concentrated under vacuum. The residue was purified by Prep-TLC (dichloromethane/methanol=12:1). The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in N-((4-((((S)-1,4-dioxan-2-yl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide (8 mg, 18.4%) as a yellow solid. LC-MS (ESI, m/z) M+1: 963. ¹HNMR (300 MHz, DMSO-d₆) δ 11.00 (d, J=6.8 Hz, 1H), 8.45-8.21 (m, 2H), 7.67-7.17 (m, 4H), 7.16-6.88 (m, 3H), 6.85-6.39 (m, 4H), 5.99 (dt, J=18.6, 2.5 Hz, 1H), 4.66 (s, 2H), 4.46 (q, J=8.6 Hz, 1H), 4.32-4.07 (m, 2H), 3.91-3.74 (m, 5H), 3.71-3.55 (m, 6H), 3.06 (dt, J=24.3, 13.1 Hz, 6H), 2.31-2.14 (m, 1H), 1.87 (d, J=65.5 Hz, 6H), 1.46 (d, J=21.7 Hz, 5H), 1.35-1.17 (m, 7H).

### Example 44: Preparation of N-((4-((((1s,4s)-4-hydroxy-4-methylcyclohexyl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide

Synthesis of N-((4-((((1s,4s)-4-hydroxy-4-methylcyclohexyl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide: Into a 40 mL vial were added 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropoxyphenyl)pyrrolidin-1-yl]-7-azaspiro[3.5]nonan-7-yl}benzoic acid (30 mg, 0.05 mmol, 1.0 eq) and 3-nitro-4-({[(1s,4s)-4-hydroxy-4-methylcyclohexyl]methyl}amino)benzenesulfonamide (16 mg, 0.05 mmol, 1.0 eq), CH₂Cl₂ (3 mL), EDCI (18 mg, 0.1 mmol, 2.0 eq), DMAP (22 mg, 0.2 mmol, 4.0 eq). The resulting mixture was stirred for overnight at 25° C. The resulting mixture was concentrated under vacuum. The residue was purified by Prep-TLC (dichloromethane/methanol=11:1). The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in N-((4-((((1s,4s)-4-hydroxy-4-methylcyclohexyl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide (13 mg, 29.1%) as a yellow solid. LC-MS (ESI, m/z) M+1: 989. ¹HNMR (300 MHz, DMSO-d₆) δ 8.37-8.22 (m, 2H), 7.62-7.45 (m, 2H), 7.45-6.30 (m, 9H), 6.01 (ddd, J=24.0, 3.4, 2.0 Hz, 1H), 4.81-4.40 (m, 2H), 4.27-3.95 (m, 3H), 3.90-3.75 (m, 2H), 3.62-3.51 (m, 2H), 3.29-2.92 (m, 9H), 2.27-1.44 (m, 19H), 1.31-1.21 (m, 7H), 1.11 (s, 5H).

### Example 45: Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide: Into a 40 mL vial were added 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(2-isopropoxyphenyl)pyrrolidin-1-yl]-7-azaspiro[3.5]nonan-7-yl}benzoic acid (30 mg, 0.05 mmol, 1.0 eq) and 3-nitro-4-({[(1r,4r)-4-hydroxy-4-methylcyclohexyl]methyl}amino)benzenesulfonamide (16 mg, 0.05 mmol, 1.0 eq), CH₂Cl₂ (3 mL), EDCI (18 mg, 0.1 mmol, 2.0 eq), DMAP (22 mg, 0.2 mmol, 4.0 eq). The resulting mixture was stirred for overnight at 25° C. The resulting mixture was concentrated under vacuum. The residue was purified by Prep-TLC (dichloromethane/methanol=11:1). The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in N-((4-((((1r,4r)-4-hydroxy-4-methylcyclohexyl)methyl)amino)-3-nitrophenyl)sulfonyl)-4-(2-((S)-2-(2-isopropoxyphenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide (13 mg, 29.1%) as a yellow solid. LC-MS (ESI, m/z) M+1: 989. ¹HNMR (300 MHz, DMSO-d₆) δ 8.37-8.22 (m, 2H), 7.62-7.45 (m, 2H), 7.45-6.30 (m, 9H), 6.00 (ddd, J=23.9, 3.3, 1.9 Hz, 1H), 4.78-4.37 (m, 2H), 4.29-4.06 (m, 3H), 3.90-3.75 (m, 2H), 3.62-3.51 (m, 2H), 3.29-2.92 (m, 9H), 2.27-1.44 (m, 19H), 1.31-1.21 (m, 7H), 1.11 (s, 5H).

### Example 46: Preparation of 4-{2-[(2S)-2-(2-tert-butylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide

Synthesis of (2-(tert-butyl)phenyl)methanol: To a stirred solution of 2-tert-butylbenzoic acid (25 g, 140.2 mmol, 1.0 eq) and THE (400 mL) were added BH₃-THF (420 mL, 420.8 mmol, 3.0 eq) dropwise at 0° C. under nitrogen atmosphere. The resulting mixture was stirred for overnight at 25° C. under nitrogen atmosphere. The reaction was quenched by the addition of CH₃OH (50 mL) at 25° C. and stirred for 2 hours. The resulting mixture was extracted with ethyl acetate (2×50 mL). The combined organic layers were washed with brine (1×50 mL), dried over anhydrous Na₂SO₄. After filtration, the filtrate was concentrated under reduced pressure. The crude residue was purified by a flash column (silica gel, ethyl acetate/petroleum ether=1:1) to afford (2-tert-butylphenyl)methanol (21 g, 91.2%) as a colorless oil. ¹HNMR (300 MHz, DMSO-d₆) δ 7.59-7.47 (m, 1H), 7.36-7.25 (m, 1H), 7.25-7.10 (m, 2H), 5.10 (t, J=5.4 Hz, 1H), 4.71 (d, J=5.1 Hz, 2H), 1.36 (s, 9H).

Synthesis of 2-(tert-butyl)benzaldehyde: To a stirred solution of (2-tert-butylphenyl)methanol (20.0 g, 121.8 mmol, 1.0 eq) and CH₂Cl₂ (400 mL) was added Dess-Martin periodate (77.5 g, 182.7 mmol, 1.5 eq) in portions at 25° C. The resulting mixture was stirred for 2 hours at 25° C. The resulting mixture was diluted with water (200 mL). The resulting mixture was extracted with CH₂Cl₂ (2×500 mL). The combined organic layers were washed with brine (1×500 mL), dried over anhydrous Na₂SO₄. After filtration, the filtrate was concentrated under reduced pressure. The crude residue was purified by a flash column (silica gel, ethyl acetate/petroleum ether=1:1) to afford 2-tert-butylbenzaldehyde (15 g, 75.9%) as a colorless oil. ¹HNMR (300 MHz, DMSO-d₆) δ 10.75 (d, J=0.7 Hz, 1H), 7.80 (dd, J=7.6, 1.6 Hz, 1H), 7.59 (td, J=8.5, 6.9, 1.6 Hz, 1H), 7.53 (dd, J=8.1, 1.7 Hz, 1H), 7.40 (qd, J=7.6, 6.9, 1.6, 0.7 Hz, 1H), 1.47 (s, 9H).

Synthesis of (S)—N-[(2-tert-butylphenyl)methylidene]-2-methylpropane-2-sulfinamide: To a stirred solution of 2-tert-butylbenzaldehyde (15.0 g, 92.5 mmol, 1.0 eq) and (S)-2-methylpropane-2-sulfinamide (11.2 g, 92.5 mmol, 1.0 eq) in THE (300 mL) were added Ti(OEt)₄ (42.2 g, 184.9 mmol, 2.0 eq) at 25° C. The resulting mixture was stirred for overnight at 55° C. The mixture was allowed to cool down to 25° C. The reaction was then quenched by water (300 mL) and then diluted with ethyl acetate (500 mL). The resulting mixture was filtered, the filter cake was washed with ethyl acetate (2×100 mL). The filtrate was concentrated under reduced pressure. This resulted in (S)—N-[(2-tert-butylphenyl)methylidene]-2-methylpropane-2-sulfinamide (16 g, 65.2%) as a colorless oil. ¹HNMR (300 MHz, DMSO-d₆) δ 9.27 (s, 1H), 8.04-7.92 (m, 1H), 7.59-7.43 (m, 2H), 7.43-7.29 (m, 1H), 1.43 (s, 9H), 1.20 (s, 9H).

Synthesis of (R)—N-[(1S)-1-(2-tert-butylphenyl)-3-(1,3-dioxan-2-yl)propyl]-2-methylpropane-2-sulfinamide: Into a 500-mL 3-necked round-bottom flask purged and maintained with an inert atmosphere of nitrogen, was placed Mg (5.5 g, 226.1 mmol, 4.0 eq), I₂ (1.4 g, 5.6 mmol, 0.1 eq), THE (150.00 mL), the resulting solution was warmed to 65° C., followed by the addition of 2-(2-bromoethyl)-1,3-dioxane (0.6 g, 22.6 mmol, 0.4 eq) in THE (20 mL) at 65° C. After addition completed, the mixture was cooled to 25° C., another 2-(2-bromoethyl)-1,3-dioxane (4.9 g, 203.4 mmol, 3.6 eq) in THE (50 mL) was added. The resulting mixture was stirred for 30 min at 25° C. under nitrogen atmosphere. To the above mixture was added (S)—N-[(2-tert-butylphenyl)methylidene]-2-methylpropane-2-sulfinamide (15.0 g, 56.5 mmol, 1.0 eq) (in 50 ml CH₂Cl₂) dropwise over 30 min at −40° C. The resulting mixture was stirred for additional 1 hour at −40° C. under nitrogen atmosphere. The reaction was then quenched by the addition of 300 mL of aqueous NH₄Cl. The resulting solution was extracted with dichloromethane (3×200 mL) and the organic layers combined. The resulting mixture was washed with brine (1×500 ml). The mixture was dried over anhydrous sodium sulfate and concentrated under vacuum. The crude residue was purified by a flash column (silica gel, ethyl acetate/petroleum ether=1:1) to afford (R)—N-[(1S)-1-(2-tert-butylphenyl)-3-(1,3-dioxan-2-yl)propyl]-2-methylpropane-2-sulfinamide (10.0 g, 46.4%) as a colorless oil. LC-MS (ES, m/z) M+1: 382.

Synthesis of (2S)-2-(2-tert-butylphenyl)pyrrolidine: Into a 500-mL round-bottom flask, was placed (R)—N-[(1S)-1-(2-tert-butylphenyl)-3-(1,3-dioxan-2-yl)propyl]-2-methylpropane-2-sulfinamide (10.0 g, 26.2 mmol, 1.0 eq), TFA (60 mL), water (15 mL), the resulting solution was stirred for 1 hour at 25° C. TFA (40 mL), triethylsilane (18.3 g, 157.2 mmol, 6.0 eq) was added. The resulting solution was stirred overnight at 60° C. The resulting mixture was cooled to 25° C. and concentrated. The resulting solution was poured into 300 mL of HCl (2.0 M). The resulting solution was extracted with MTBE (2×200 mL) and the organic layers combined. The resulting mixture was washed with of HCl (1×200 ml, 2.0 M). The pH value of the combined aqueous was adjusted to 13 with NaOH (4.0 M). The resulting solution was extracted with dichloromethane (3×200 mL) and the organic layers combined and dried over anhydrous sodium sulfate and concentrated. This resulted in (2S)-2-(2-tert-butylphenyl)pyrrolidine (4.0 g, 75.1%) as a colorless oil. LC-MS (ESI, m/z) M+1: 204. ¹HNMR (300 MHz, Chloroform-d) δ 7.57 (dd, J=7.7, 1.7 Hz, 1H), 7.38 (dd, J=7.8, 1.6 Hz, 1H), 7.32-7.13 (m, 2H), 4.80-4.68 (m, 1H), 3.35 (td, J=10.0, 7.4, 4.9 Hz, 1H), 3.09-2.95 (m, 1H), 2.31-2.16 (m, 1H), 2.12-1.85 (m, 2H), 1.82-1.65 (m, 1H), 1.48 (s, 9H).

Synthesis of 4-{2-[(2S)-2-(2-tert-butylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq) and (2S)-2-(2-tert-butylphenyl)pyrrolidine (22 mg, 0.1 mmol, 1.0 eq), CH₂Cl₂ (3 mL), NaBH(OAc)₃ (47 mg, 0.2 mmol, 2.0 eq). The resulting mixture was stirred for overnight at 25° C. The reaction was then quenched by the addition of water (10 mL). The resulting solution was extracted with dichloromethane (2×20 mL). The resulting mixture was washed with brine (1×50 ml). The organic layers were combined and dried over anhydrous sodium sulfate and concentrated under vacuum. The residue was purified by Prep-TLC (dichloromethane/methanol=12:1) to afford 4-{2-[(2S)-2-(2-tert-butylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (80 mg, 66.2%) as a yellow solid. LC-MS (ESI, m/z) M+1: 1089.

Synthesis of 4-{2-[(2S)-2-(2-tert-butylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide: Into a 100 mL round-bottom flask were added 4-{2-[(2S)-2-(2-tert-butylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (80 mg, 0.07 mmol, 1.0 eq), TBAF in THE (1.0 M, 5 mL), ethylenediamine (88 mg, 1.5 mmol, 20.0 eq). The resulting mixture was stirred for 5 hours at 70° C. The mixture was allowed to cool down to 25° C. The resulting mixture was diluted with water (50 mL). The resulting mixture was extracted with ethyl acetate (3×30 mL). The combined organic layers were washed with brine (1×100 mL), dried over anhydrous Na₂SO₄. After filtration, the filtrate was concentrated under reduced pressure. The residue was purified by Prep-TLC (dichloromethane/methanol=11:1). The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in 4-{2-[(2S)-2-(2-tert-butylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide (8 mg, 11.4%) as a yellow solid. LC-MS (ESI, m/z) M+1: 959. ¹HNMR (300 MHz, DMSO-d₆) δ 12.24 (s, 1H), 11.08 (d, J=46.9 Hz, 1H), 8.35 (dd, J=27.1, 11.4 Hz, 2H), 7.80 (s, 1H), 7.60-7.00 (m, 7H), 6.87-6.46 (m, 4H), 6.01 (d, J=21.5 Hz, 1H), 4.76-4.02 (m, 3H), 3.95-3.80 (m, 6H), 3.06-2.93 (m, 6H), 2.35-2.10 (m, 3H), 2.00-1.56 (m, 9H), 1.50-1.10 (m, 20H).

### Example 47: Preparation of N-((3-nitro-4-(((tetrahydro-2H-pyran-4-yl)methyl)amino)phenyl)sulfonyl)-4-(2-((S)-2-(2-(prop-1-yn-1-yl)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide

Synthesis of tert-butyl (2S)-2-(2-bromophenyl)pyrrolidine-1-carboxylate: Into a 250-mL round-bottom flask, was placed (2S)-2-(2-bromophenyl)pyrrolidine (17.0 g, 75.2 mmol, 1.0 eq), CH₂Cl₂ (200 mL), Boc₂O (25.0 g, 114.5 mmol, 1.5 eq), TEA (15.3 g, 151.2 mmol, 2.0 eq), DMAP (922 mg, 7.5 mmol, 0.1 eq). The resulting solution was stirred overnight at 25° C. The resulting mixture was concentrated under vacuum. The crude residue was purified by a flash column (silica gel, ethyl acetate/petroleum ether=1:15). This resulted in tert-butyl (2S)-2-(2-bromophenyl)pyrrolidine-1-carboxylate (24 g, 99.5%) as a white solid. LC-MS (ESI, m/z) M-t-Bu+CH₃CN: 311/313. ¹HNMR (300 MHz, Chloroform-d) δ 7.54 (dd, J=8.0, 1.3 Hz, 1H), 7.27 (d, J=7.3 Hz, 1H), 7.21-7.05 (m, 2H), 5.30-5.07 (m, 1H), 3.81-3.43 (m, 2H), 2.42 (dq, J=17.5, 9.9, 8.7 Hz, 1H), 1.97-1.77 (m, 2H), 1.49 (d, J=3.8 Hz, 3H), 1.21 (s, 6H).

Synthesis of tert-butyl (2S)-2-[2-(prop-1-yn-1-yl)phenyl]pyrrolidine-1-carboxylate: Into a 40 mL vial were added tert-butyl (2S)-2-(2-bromophenyl)pyrrolidine-1-carboxylate (300 mg, 0.9 mmol, 1.0 eq), DMSO (5 mL), 2-butynoic acid (101 mg, 1.2 mmol, 1.3 eq), DPPB (79 mg, 0.2 mmol, 0.2 eq), DBU (421 mg, 2.8 mmol, 3.0 eq), Pd(PPh₃)₂Cl₂ (67 mg, 0.1 mmol, 0.1 eq). The resulting mixture was stirred for overnight at 110° C. under nitrogen atmosphere. The mixture was allowed to cool down to 25° C. The resulting mixture was diluted with water (200 mL). The resulting mixture was extracted with Ethyl acetate (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1:7) to afford tert-butyl (2S)-2-[2-(prop-1-yn-1-yl)phenyl]pyrrolidine-1-carboxylate (250 mg, 95.3%) as a light yellow oil. LC-MS (ES, m/z) M-t-Bu+1: 230.

Synthesis of (2S)-2-[2-(prop-1-yn-1-yl)phenyl]pyrrolidine hydrochloride: A solution of tert-butyl (2S)-2-[2-(prop-1-yn-1-yl)phenyl]pyrrolidine-1-carboxylate (133 mg, 0.5 mmol, 1.0 eq) in HCl (gas) in 1,4-dioxane (4 M, 3 mL) was stirred for 1 h at 25° C. The resulting mixture was concentrated under vacuum. This resulted in (2S)-2-[2-(prop-1-yn-1-yl)phenyl]pyrrolidine hydrochloride (100 mg, 96.7%) as a light yellow oil. LC. MS (ES, m/z) M+1: 186. ¹HNMR (300 MHz, Methanol-d₄) δ 7.55 (ddd, J=14.9, 7.0, 1.9 Hz, 2H), 7.42 (pd, J=7.4, 1.7 Hz, 2H), 5.12 (d, J=15.6 Hz, 1H), 3.50 (t, J=6.8 Hz, 2H), 2.56-2.20 (m, 4H), 2.16 (s, 3H).

Synthesis of N-{3-nitro-4-[(oxan-4-ylmethyl)amino]benzenesulfonyl}-4-{2-[(2S)-2-[2-(prop-1-yn-1-yl)phenyl]pyrrolidin-1-yl]-7-azaspiro[3.5]nonan-7-yl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: To a stirred solution of N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq) and (2S)-2-[2-(prop-1-yn-1-yl)phenyl]pyrrolidine hydrochloride (25 mg, 0.1 mmol, 1.0 eq) in CH₂Cl₂ (3 mL) was added NaBH(OAc)₃ (47 mg, 0.2 mmol, 2.0 eq). The resulting mixture was stirred for overnight at 25° C. The resulting mixture was concentrated under vacuum. The residue was purified by Prep-TLC (dichloromethane/methanol=13:1) to afford N-{3-nitro-4-[(oxan-4-ylmethyl)amino]benzenesulfonyl}-4-{2-[(2S)-2-[2-(prop-1-yn-1-yl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (90 mg, 75.8%) as a yellow solid. LC-MS (ES, m/z) M+1: 1071.

Synthesis of N-((3-nitro-4-(((tetrahydro-2H-pyran-4-yl)methyl)amino)phenyl)sulfonyl)-4-(2-((S)-2-(2-(prop-1-yn-1-yl)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide: Into a 100 mL round-bottom flask were added N-{3-nitro-4-[(oxan-4-ylmethyl)amino]benzenesulfonyl}-4-{2-[(2S)-2-[2-(prop-1-yn-1-yl)phenyl]pyrrolidin-1-yl]-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (90 mg, 0.08 mmol, 1.0 eq), TBAF in THE (1.0 M, 5 mL), ethylenediamine (101 mg, 1.6 mmol, 20.0 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in N-((3-nitro-4-(((tetrahydro-2H-pyran-4-yl)methyl)amino)phenyl)sulfonyl)-4-(2-((S)-2-(2-(prop-1-yn-1-yl)phenyl)pyrrolidin-1-yl)-7-azaspiro[3.5]nonan-7-yl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide (15 mg, 18.9%) as a yellow solid. LC-MS (ES, m/z) M+1: 941. ¹HNMR (300 MHz, DMSO-d₆) δ 12.26 (s, 1H), 11.08 (d, J=46.9 Hz, 1H), 8.35 (dd, J=27.1, 11.4 Hz, 2H), 7.66-7.03 (m, 7H), 6.94-6.49 (m, 4H), 6.02 (dt, J=15.5, 2.1 Hz, 1H), 4.73-4.43 (m, 1H), 4.35-4.02 (m, 2H), 3.98-3.66 (m, 5H), 3.61-3.47 (m, 1H), 3.12 (d, J=26.7 Hz, 10H), 2.29-2.13 (m, 2H), 2.08 (s, 3H), 1.54 (ddd, J=111.2, 55.5, 19.0 Hz, 17H).

### Example 50: Preparation of N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((R)-3-(o-tolyl)morpholino)-7-azaspiro[3.5]nonan-7-yl)benzamide

Synthesis of N-((3-nitro-4-(((tetrahydro-2H-pyran-4-yl)methyl)amino)phenyl)sulfonyl)-4-(2-((R)-3-(o-tolyl)morpholino)-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq) and (R)-3-(o-tolyl)morpholine (22 mg, 0.1 mmol, 1.0 eq), CH₂Cl₂ (3 mL), NaBH(OAc)₃ (47 mg, 0.2 mmol, 2.0 eq). The resulting mixture was stirred for overnight at 25° C. The reaction was then quenched by the addition of water (10 mL). The resulting solution was extracted with dichloromethane (2×20 mL). The resulting mixture was washed with brine (1×50 ml). The organic layers were combined and dried over anhydrous sodium sulfate and concentrated under vacuum. The residue was purified by Prep-TLC (dichloromethane/methanol=12:1) to afford N-((3-nitro-4-(((tetrahydro-2H-pyran-4-yl)methyl)amino)phenyl)sulfonyl)-4-(2-((R)-3-(o-tolyl)morpholino)-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (80 mg, 66.2%) as a yellow solid. LC-MS (ES, m/z) M+1: 1063.

Synthesis of N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((R)-3-(o-tolyl)morpholino)-7-azaspiro[3.5]nonan-7-yl)benzamide: Into a 100 mL round-bottom flask were added N-((3-nitro-4-(((tetrahydro-2H-pyran-4-yl)methyl)amino)phenyl)sulfonyl)-4-(2-((R)-3-(o-tolyl)morpholino)-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (80 mg, 0.07 mmol, 1.0 eq), TBAF in THE (1.0 M, 5 mL), ethylenediamine (88 mg, 1.5 mmol, 20.0 eq). The resulting mixture was stirred for 5 hours at 70° C. The mixture was allowed to cool down to 25° C. The resulting mixture was diluted with water (50 mL). The resulting mixture was extracted with ethyl acetate (3×30 mL). The combined organic layers were washed with brine (1×100 mL), dried over anhydrous Na₂SO₄. After filtration, the filtrate was concentrated under reduced pressure. The residue was purified by Prep-TLC (dichloromethane/methanol=11:1). The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((R)-3-(o-tolyl)morpholino)-7-azaspiro[3.5]nonan-7-yl)benzamide (8 mg, 11.4%) as a yellow solid. LC-MS (ES, m/z) M+1: 933. ¹H NMR (400 MHz, DMSO-d₆) δ 12.30 (s, 1H), 11.22 (s, 1H), 8.55 (s, 1H), 8.31 (s, 1H), 7.62 (d, J=9.2 Hz, 1H), 7.11 (s, 5H), 6.87 (s, 1H), 6.78 (d, J=13.7 Hz, 2H), 6.57 (s, 1H), 6.03 (d, J=16.0 Hz, 1H), 4.62 (d, J=9.1 Hz, 1H), 4.32 (s, 2H), 3.87 (d, J=12.0 Hz, 5H), 3.57 (d, J=13.3 Hz, 1H), 3.51 (s, 1H), 3.10 (s, 6H), 2.91 (s, 2H), 2.85 (d, J=11.5 Hz, 1H), 2.55 (s, 1H), 2.34 (s, 5H), 2.19 (s, 2H), 1.89 (s, 3H), 1.71 (s, 2H), 1.62 (d, J=12.7 Hz, 5H), 1.32-1.22 (m, 2H), 1.14 (s, 1H).

### Example 51: Preparation of N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-3-(o-tolyl)morpholino)-7-azaspiro[3.5]nonan-7-yl)benzamide

Synthesis of N-((3-nitro-4-(((tetrahydro-2H-pyran-4-yl)methyl)amino)phenyl)sulfonyl)-4-(2-((S)-3-(o-tolyl)morpholino)-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq) and (S)-3-(o-tolyl)morpholine (22 mg, 0.1 mmol, 1.0 eq), CH₂Cl₂ (3 mL), NaBH(OAc)₃ (47 mg, 0.2 mmol, 2.0 eq). The resulting mixture was stirred for overnight at 25° C. The reaction was then quenched by the addition of water (10 mL). The resulting solution was extracted with dichloromethane (2×20 mL). The resulting mixture was washed with brine (1×50 ml). The organic layers were combined and dried over anhydrous sodium sulfate and concentrated under vacuum. The residue was purified by Prep-TLC (dichloromethane/methanol=12:1) to afford N-((3-nitro-4-(((tetrahydro-2H-pyran-4-yl)methyl)amino)phenyl)sulfonyl)-4-(2-((S)-3-(o-tolyl)morpholino)-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (80 mg, 66.2%) as a yellow solid. LC-MS (ES, m/z) M+1: 1063.

Synthesis of N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-3-(o-tolyl)morpholino)-7-azaspiro[3.5]nonan-7-yl)benzamide: Into a 100 mL round-bottom flask were added N-((3-nitro-4-(((tetrahydro-2H-pyran-4-yl)methyl)amino)phenyl)sulfonyl)-4-(2-((S)-3-(o-tolyl)morpholino)-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (80 mg, 0.07 mmol, 1.0 eq), TBAF in THE (1.0 M, 5 mL), ethylenediamine (88 mg, 1.5 mmol, 20.0 eq). The resulting mixture was stirred for 5 hours at 70° C. The mixture was allowed to cool down to 25° C. The resulting mixture was diluted with water (50 mL). The resulting mixture was extracted with ethyl acetate (3×30 mL). The combined organic layers were washed with brine (1×100 mL), dried over anhydrous Na₂SO₄. After filtration, the filtrate was concentrated under reduced pressure. The residue was purified by Prep-TLC (dichloromethane/methanol=11:1). The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((S)-3-(o-tolyl)morpholino)-7-azaspiro[3.5]nonan-7-yl)benzamide (8 mg, 11.4%) as a yellow solid. LC-MS (ES, m/z) M+1: 933. ¹HNMR (400 MHz, DMSO-d₆) δ 12.30 (s, 1H), 11.22 (s, 1H), 8.55 (s, 1H), 8.31 (s, 1H), 7.62 (d, J=9.2 Hz, 1H), 7.11 (s, 5H), 6.87 (s, 1H), 6.78 (d, J=13.7 Hz, 2H), 6.57 (s, 1H), 6.03 (d, J=16.0 Hz, 1H), 4.62 (d, J=9.1 Hz, 1H), 4.32 (s, 2H), 3.87 (d, J=12.0 Hz, 5H), 3.57 (d, J=13.3 Hz, 1H), 3.51 (s, 1H), 3.10 (s, 6H), 2.91 (s, 2H), 2.85 (d, J=11.5 Hz, 1H), 2.55 (s, 1H), 2.34 (s, 5H), 2.19 (s, 2H), 1.89 (s, 3H), 1.71 (s, 2H), 1.62 (d, J=12.7 Hz, 5H), 1.32-1.22 (m, 2H), 1.14 (s, 1H).

### Example 54: Preparation of 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide

Synthesis of (3R,4R)-4-bromooxolan-3-ol: A solution of NBS (660.0 g, 3709.5 mmol, 1.3 eq) in water (3000 mL) was treated with 2,5-dihydrofuran (200.0 g, 2853.4 mmol, 1.0 eq) for 1 hour at 25° C. The resulting mixture was stirred for 16 hours at 25° C. The resulting mixture was extracted with Ethyl acetate (2×2 L). The combined organic layers were washed with NaHSO₃ (1×2 L), brine (1×2 L) dried over anhydrous Na₂SO₄. After filtration, the filtrate was concentrated under reduced pressure. This resulted in (3R,4R)-4-bromooxolan-3-ol (400 g, 83.94%) as a yellow oil. ¹HNMR (400 MHz, DMSO-d₆) δ 4.42-4.34 (m, 1H), 4.31 (dt, J=3.1, 1.5 Hz, 1H), 4.22 (dd, J=10.8, 4.1 Hz, 1H), 4.08 (dd, J=9.5, 4.5 Hz, 1H), 3.92 (dd, J=10.7, 1.6 Hz, 1H), 3.58 (dd, J=9.5, 1.7 Hz, 1H).

Synthesis of (3R,4R)-4-bromooxolan-3-yl 2-phenylacetate (trans-racemate): A solution of (3R,4R)-4-bromooxolan-3-ol (360.0 g, 2155.7 mmol, 1.0 eq) in CH₃CN (3 L) was treated with K₂CO₃ (595.9 g, 4311.3 mmol, 2.0 eq) at 25° C. under nitrogen atmosphere followed by the addition of phenylacetyl chloride (366.6 g, 2371.2 mmol, 1.1 eq) dropwise at 25° C. The resulting mixture was stirred for 3 hours at 25° C. under nitrogen atmosphere. The reaction was quenched by the addition of Water/Ice (1 L) at 25° C. The resulting mixture was extracted with Ethyl acetate (2×3 L). The combined organic layers were washed with brine (1×3 L), dried over anhydrous Na₂SO₄. After filtration, the filtrate was concentrated under reduced pressure. The residue was purified by silica gel column chromatography, eluted with ethyl acetate/petroleum ether (1:1) to afford (3R,4R)-4-bromooxolan-3-yl 2-phenylacetate (300.0 g, 48.8%) (trans-racemate) as a yellow oil. ¹HNMR (400 MHz, Chloroform-d) δ 7.49-7.18 (m, 5H), 5.40 (dt, J=4.5, 1.4 Hz, 1H), 4.39-4.23 (m, 2H), 4.27-4.21 (m, 1H), 4.08 (dd, J=10.2, 1.8 Hz, 1H), 3.87 (dd, J=10.6, 1.5 Hz, 1H), 3.66 (s, 2H).

Synthesis of (3R,4R)-4-bromooxolan-3-yl 2-phenylacetate (cis-racemate): A mixture of (3R,4R)-4-bromooxolan-3-yl 2-phenylacetate (trans-racemate) (50.0 g, 175.4 mmol, 1.0 eq) and NaN₃ (22.8 g, 350.7 mmol, 2.0 eq) in DMSO (1.0 L) was stirred for 16 hours at 100° C. under nitrogen atmosphere. The mixture was allowed to cool down to 25° C. The resulting mixture was diluted with water (2 L). The resulting mixture was extracted with Ethyl acetate (2×1 L). The combined organic layers were washed with brine (1×2 L), dried over anhydrous Na₂SO₄. After filtration, the filtrate was concentrated under reduced pressure to afford (3S,4S)-4-azidooxolan-3-yl 2-phenylacetate (70.0 g crude, 80.7%) (cis-racemate) as a yellow oil. This crude was used directly for next step without further purification.

Synthesis of N-[(3S,4S)-4-hydroxyoxolan-3-yl]-2-phenylacetamide (cis-racemate): A mixture of (3S,4S)-4-azidooxolan-3-yl 2-phenylacetate (cis-racemate) (70.0 g, 141.6 mmol, 1.0 eq, 50% Purity) and Pd/C (3.0 g, 28.3 mmol, 0.2 eq) in THE (1.0 L) was stirred for 12 hours at 25° C. under hydrogen atmosphere. The resulting mixture was filtered, the filter cake was washed with Ethyl acetate (3×300 mL). The filtrate was concentrated under reduced pressure. The residue was purified by silica gel column chromatography, eluted with ethyl acetate/petroleum ether (1:1) to afford N-[(3S,4S)-4-hydroxyoxolan-3-yl]-2-phenylacetamide (13.0 g, 41.5%) (cis-racemate) as a white solid. ¹HNMR (300 MHz, DMSO-d₆) δ 7.83 (d, J=6.9 Hz, 1H), 7.36-7.15 (m, 5H), 5.28 (d, J=4.2 Hz, 1H), 4.23-4.10 (m, 2H), 3.94-3.77 (m, 2H), 3.60 (dd, J=9.5, 1.9 Hz, 1H), 3.51 (d, J=1.4 Hz, 2H), 3.40 (t, J=8.0 Hz, 1H).

Synthesis of N-[(3S,4S)-4-[(5-bromo-1-{[2-(trimethylsilyl)ethoxy]methyl}pyrrolo[2,3-b]pyridin-6-yl)oxy]oxolan-3-yl]-2-phenylacetamide (cis-racemate): A solution of N-[(3S,4S)-4-hydroxyoxolan-3-yl]-2-phenylacetamide (1.92 g, 8.688 mmol, 1.5 eq) (cis-racemate) in THE was treated with NaH (0.4 g, 17.4 mmol, 3.0 eq) for 5 min at 25° C. under nitrogen atmosphere, The resulting mixture was stirred for 30 min at 25° C. under nitrogen atmosphere. This was followed by the addition of 5-bromo-6-fluoro-1-{[2-(trimethylsilyl)ethoxy]methyl}pyrrolo[2,3-b]pyridine (2.0 g, 5.8 mmol, 1.0 eq) at 25° C. The resulting mixture was stirred for 4 hours at 50° C. under nitrogen atmosphere. The mixture was allowed to cool down to 25° C. and then quenched by the addition of Water (20 mL) at 25° C. The resulting mixture was extracted with Ethyl acetate (2×50 mL). The combined organic layers were washed with brine (1×100 mL), dried over anhydrous Na₂SO₄. After filtration, the filtrate was concentrated under reduced pressure. The residue was purified by silica gel column chromatography, eluted with ethyl acetate/petroleum ether (1:1) to afford N-[(3S,4S)-4-[(5-bromo-1-{[2-(trimethylsilyl)ethoxy]methyl}pyrrolo[2,3-b]pyridin-6-yl)oxy]oxolan-3-yl]-2-phenylacetamide (2.0 g, 63.2%) (cis-racemate) as a white solid. LC-MS (ESI, m/z) M+1: 546/548. ¹HNMR (300 MHz, DMSO-d₆) δ 8.24 (s, 1H), 8.08 (d, J=7.6 Hz, 1H), 7.42 (d, J=3.6 Hz, 1H), 7.21-7.03 (m, 5H), 6.44 (d, J=3.6 Hz, 1H), 5.57-5.47 (m, 2H), 5.34 (d, J=10.9 Hz, 1H), 4.66-4.50 (m, 1H), 4.26 (dd, J=10.3, 4.9 Hz, 1H), 4.04 (dd, J=8.3, 7.4 Hz, 1H), 3.85 (dd, J=10.3, 2.9 Hz, 1H), 3.69 (t, J=8.3 Hz, 1H), 3.49 (t, J=8.0 Hz, 2H), 3.41 (d, J=14.3 Hz, 1H), 3.33 (d, J=14.2 Hz, 1H), 2.08 (s, 1H), 0.92-0.70 (m, 2H), −0.11 (s, 9H).

Synthesis of 2-phenyl-1-[(11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraen-10-yl]ethanone (cis-racemate): A mixture of N-[(3S,4S)-4-[(5-bromo-1-{[2-(trimethylsilyl)ethoxy]methyl}pyrrolo[2,3-b]pyridin-6-yl)oxy]oxolan-3-yl]-2-phenylacetamide (cis-racemate) (1.9 g, 3.5 mmol, 1.0 eq), CuI (0.3 g, 1.4 mmol, 0.4 eq), 2,2,6,6-tetramethylheptane-3,5-dione (0.3 g, 1.4 mmol, 0.4 eq) and Cs₂CO₃ (2.3 g, 6.9 mmol, 2.0 eq) in DMF (40 mL) was stirred for 2 hours at 100° C. under nitrogen atmosphere. The mixture was allowed to cool down to 25° C. The resulting mixture was diluted with water (50 mL). The resulting mixture was extracted with Ethyl acetate (2×50 mL). The combined organic layers were washed with water (2×80 mL), brine (1×100 mL), dried over anhydrous Na₂SO₄. After filtration, the filtrate was concentrated under reduced pressure. The residue was purified by silica gel column chromatography, eluted with ethyl acetate/petroleum (1:1) to afford 2-phenyl-1-[(11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraen-10-yl]ethanone (1.5 g, 92.7%) (cis-racemate) as a colorless oil. LC-MS (ESI, m/z) M+1: 466.

Synthesis of (11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraene (cis-racemate): A solution of 2-phenyl-1-[(11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraen-10-yl]ethanone (cis-racemate) (1.5 g, 3.2 mmol, 1.0 eq) and LiOH (0.3 g, 12.9 mmol, 4.0 eq) in THE (15 mL), water (15 mL) was stirred for 40 h at 70° C., The mixture was allowed to cool down to 25° C. The resulting mixture was diluted with water (20 mL). The resulting mixture was extracted with Ethyl acetate (2×50 mL). The combined organic layers were washed with brine (1×50 mL), dried over anhydrous Na₂SO₄. After filtration, the filtrate was concentrated under reduced pressure. The residue was purified by silica gel column chromatography, eluted with ethyl acetate/petroleum (1:1) to afford (11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraene (800 mg, 71.5%) (cis-racemate) as a colorless oil. LC-MS (ESI, m/z) M+1: 348.

Synthesis of (11R,15R)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraene: 700 mg of (11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraene (800 mg, 71.5%) (cis-racemate) was separated by SFC with the following conditions: Column: Lux Cellulose-4 50*2.0 mm, 3.0 um; co-solvent: MeOH; Flow rate: 3.0 ml/min; Gradient: 10% B to 50% B in 3 min, RT: 0.556 min; Detector, 220 nm. This resulted in (11R,15R)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraene (280 mg, 40.0%) as a colorless oil. LC. MS (ESI, m/z) M+1: 348. ¹HNMR (300 MHz, DMSO-d₆) δ 7.27 (d, J=3.5 Hz, 1H), 7.16 (s, 1H), 6.25 (d, J=3.5 Hz, 1H), 5.98 (d, J=4.7 Hz, 1H), 5.41 (s, 2H), 4.50 (td, J=4.2, 1.6 Hz, 1H), 4.10 (dd, J=10.1, 4.0 Hz, 1H), 4.02 (tt, J=8.5, 4.5 Hz, 1H), 3.99-3.83 (m, 2H), 3.47 (t, J=8.0 Hz, 2H), 3.36 (d, J=8.3 Hz, 1H), 0.86-0.75 (m, 2H), −0.09 (s, 9H).

Synthesis of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(11R,15R)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: A mixture of 2-bromo-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide (80 mg, 0.10 mmol, 1.0 eq), (11R,15R)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (34 mg, 0.10 mmol, 1.0 eq), ethanediamide, N,N′-diphenyl-(5 mg, 0.02 mmol, 0.2 eq), CuI (4 mg, 0.02 mmol, 0.2 eq) and Cs₂CO₃ (15 mg, 0.2 mmol, 2.0 eq) in DMF (3 mL) was stirred for 2 hours at 100° C. under nitrogen atmosphere. The mixture was allowed to cool down to 25° C. The resulting mixture was diluted with water (5 mL). The resulting mixture was extracted with Ethyl acetate (2×10 mL). The combined organic layers were washed with brine (1×20 mL), dried over anhydrous Na₂SO₄. After filtration, the filtrate was concentrated under reduced pressure. The residue was purified by Prep-TLC (CH₂Cl₂/MeOH=10:1) to afford 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(11R,15R)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (50 mg, 47.2%) as a yellow solid. LC-MS (ESI, m/z) (M/2)+1: 546.

Synthesis of 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide: A solution of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(11R,15R)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (50 mg, 0.05 mmol, 1.0 eq) in TBAF (2 mL, 1M/L in THF) was treated with ethylenediamine (55 mg, 0.9 mmol, 20.0 eq) at 25° C. The resulting mixture was stirred for 4 hours at 70° C. The mixture was allowed to cool down to 25° C. The resulting mixture was diluted with water (10 mL). The resulting mixture was extracted with Ethyl acetate (2×20 mL). The combined organic layers were washed with brine (1×20 mL), dried over anhydrous Na₂SO₄. After filtration, the filtrate was concentrated under reduced pressure. The crude product (20 mg) was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min) detector, UV 254 nm.) to afford 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aR)-5a,6,8,8a-tetrahydrofuro[3,4-b]pyrrolo[3′,2′:5,6]pyrido[3,2-e][1,4]oxazin-5(1H)-yl)benzamide (5 mg, 11.0%) as a yellow solid. LC-MS (ESI, m/z) M+1: 961. ¹HNMR (400 MHz, DMSO-d₆) δ 11.85 (s, 1H), 11.03 (s, 1H), 10.38 (s, 1H), 8.54 (t, J=5.9 Hz, 1H), 8.35 (s, 1H), 7.75-7.67 (m, 1H), 7.58 (s, 1H), 7.41-7.33 (m, 1H), 7.30 (d, J=9.6 Hz, 1H), 7.09 (d, J=8.3 Hz, 1H), 7.05 (s, 1H), 6.99 (t, J=7.5 Hz, 1H), 6.95-6.88 (m, 1H), 6.66 (d, J=9.4 Hz, 1H), 6.54 (s, 1H), 5.98 (s, 1H), 4.78-4.67 (m, 2H), 4.29 (s, 1H), 4.09-3.99 (m, 2H), 3.87 (dt, J=13.0, 6.3 Hz, 4H), 3.29 (q, J=9.4, 7.2 Hz, 6H), 3.15 (s, 4H), 2.31 (s, 1H), 2.15 (d, J=8.8 Hz, 5H), 1.90 (s, 2H), 1.74 (s, 1H), 1.69-1.58 (m, 2H), 1.50 (s, 1H), 1.33 (d, J=6.4 Hz, 6H), 1.33-1.22 (m, 4H).

### Example 55: Preparation of 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide

Synthesis of (11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraene: 700 mg of (11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraene (800 mg, 71.5%) (cis-racemate) was separated by SFC with the following conditions: Column: Lux Cellulose-4 50*2.0 mm, 3.0 um; co-solvent: MeOH; Flow rate: 3.0 ml/min; Gradient: 10% B to 50% B in 3 min, RT: 0.654 min; Detector, 220 nm. This resulted in (11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1,3(7),5,8-tetraene (250 mg, 35.7%) as a colorless oil. LC-MS (ESI, m/z) M+1: 348. ¹HNMR (300 MHz, DMSO-d₆) δ 7.27 (d, J=3.5 Hz, 1H), 7.16 (s, 1H), 6.25 (d, J=3.5 Hz, 1H), 5.98 (d, J=4.7 Hz, 1H), 5.41 (s, 2H), 4.50 (td, J=4.2, 1.6 Hz, 1H), 4.10 (dd, J=10.1, 4.0 Hz, 1H), 4.02 (tt, J=8.5, 4.5 Hz, 1H), 3.99-3.83 (m, 2H), 3.47 (t, J=8.0 Hz, 2H), 3.36 (d, J=8.3 Hz, 1H), 0.86-0.75 (m, 2H), −0.09 (s, 9H).

Synthesis of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(11S,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: A mixture of 2-bromo-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide (80 mg, 0.1 mmol, 1.0 eq), (11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (34 mg, 0.10 mmol, 1.0 eq), ethanediamide, N,N′-diphenyl-(5 mg, 0.02 mmol, 0.2 eq), CuI (4 mg, 0.02 mmol, 0.2 eq) and Cs₂CO₃ (15 mg, 0.2 mmol, 2.0 eq) in DMF (3 mL) was stirred for 2 hours at 100° C. under nitrogen atmosphere. The mixture was allowed to cool down to 25° C. The resulting mixture was diluted with water (5 mL). The resulting mixture was extracted with Ethyl acetate (2×10 mL). The combined organic layers were washed with brine (1×20 mL), dried over anhydrous Na₂SO₄. After filtration, the filtrate was concentrated under reduced pressure. The residue was purified by Prep-TLC (CH₂Cl₂/MeOH=10:1) to afford 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (50 mg, 47.2%) as a yellow solid. LC-MS (ESI, m/z) (M/2)+1: 546.

Synthesis of 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide: A solution of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(11S,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (50 mg, 0.05 mmol, 1.0 eq) in TBAF (2 mL, 1M in THF) was treated with ethylenediamine (55 mg, 0.9 mmol, 20.0 eq) at 25° C. The resulting mixture was stirred for 4 hours at 70° C. The mixture was allowed to cool down to 25° C. The resulting mixture was diluted with water (10 mL). The resulting mixture was extracted with Ethyl acetate (2×20 mL). The combined organic layers were washed with brine (1×20 mL), dried over anhydrous Na₂SO₄. After filtration, the filtrate was concentrated under reduced pressure. The crude product (20 mg) was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min) detector, UV 254 nm.) to afford 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,8aS)-5a,6,8,8a-tetrahydrofuro[3,4-b]pyrrolo[3′,2′:5,6]pyrido[3,2-e][1,4]oxazin-5(1H)-yl)benzamide (5 mg, 11.0%) as a yellow solid. LC-MS (ESI, m/z) M+1: 961. ¹HNMR (400 MHz, DMSO-d₆) δ 10.96 (s, 1H), 8.26 (s, 2H), 7.61 (d, J=8.7 Hz, 1H), 7.49 (d, J=7.6 Hz, 1H), 7.11 (s, 1H), 7.03 (s, 2H), 6.86 (s, 3H), 6.50 (s, 2H), 6.31 (s, 1H), 5.96 (s, 2H), 4.92 (s, 1H), 4.67-4.59 (m, 2H), 4.19 (s, 1H), 4.13-4.04 (m, 1H), 4.02-3.99 (m, 1H), 3.87 (d, J=10.1 Hz, 5H), 3.53 (s, 2H), 3.09 (d, J=41.6 Hz, 10H), 2.33 (s, 2H), 2.18 (s, 3H), 2.02-1.98 (m, 1H), 1.88-1.86 (m, 3H), 1.57-1.54 (m, 6H), 1.27-1.24 (m, 7H).

### Example 58: Preparation of 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide

Synthesis of N-((3S,4S)-4-hydroxytetrahydro-2H-pyran-3-yl)-4-methylbenzenesulfonamide: A solution of (3S,4S)-3-aminotetrahydro-2H-pyran-4-ol (1.0 g, 8.5 mmol, 1.0 eq) in DCM (20 mL) was treated with TEA (2.1 g, 21.3 mmol, 2.5 eq) at 25° C. under nitrogen atmosphere followed by the addition of TsCl (1.6 g, 9.4 mmol, 1.1 eq) dropwise at 0° C. The resulting mixture was stirred for 4 hours at 25° C. under nitrogen atmosphere. The reaction was quenched by the addition of water (10 mL) at 0° C. The resulting mixture was extracted with CH₂Cl₂ (2×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford N-((3S,4S)-4-hydroxytetrahydro-2H-pyran-3-yl)-4-methylbenzenesulfonamide (1200 mg, 51.8%) as a white solid. LC-MS (ESI, m/z) M+1: 272. ¹HNMR (400 MHz, DMSO-d₆) δ 7.76-7.70 (m, 2H), 7.63 (d, J=6.9 Hz, 1H), 7.37 (d, J=8.1 Hz, 2H), 4.79 (d, J=4.7 Hz, 1H), 3.68 (dt, J=11.5, 4.4 Hz, 1H), 3.60 (dd, J=11.3, 4.1 Hz, 1H), 3.39 (tt, J=8.5, 4.3 Hz, 1H), 3.27 (ddd, J=11.4, 9.7, 2.9 Hz, 1H), 2.98 (dd, J=11.3, 8.3 Hz, 1H), 2.79 (qd, J=7.8, 4.1 Hz, 1H), 2.38 (s, 3H), 1.83 (dtd, J=13.3, 4.5, 3.0 Hz, 1H), 1.33 (dtd, J=13.4, 9.3, 4.2 Hz, 1H).

Synthesis of N-[(3S,4S)-4-[(5-bromo-1-{[2-(trimethylsilyl)ethoxy]methyl}pyrrolo[2,3-b]pyridin-6-yl)oxy]oxan-3-yl]-4-methylbenzenesulfonamide: Into a 100 mL round-bottom flask were added N-[(3S,4S)-4-hydroxyoxan-3-yl]-4-methylbenzenesulfonamide (1.7 g, 6.3 mmol, 1.0 eq), THE (15 mL). To the above solution was added NaH (766 mg, 19.1 mmol, 3.0 eq, 60%) at 0° C. The mixture was stirred for 30 min at 0° C. 5-bromo-6-fluoro-1-{[2-(trimethylsilyl)ethoxy]methyl}pyrrolo[2,3-b]pyridine (2.2 g, 6.4 mmol, 1.0 eq) was added and the mixture was allowed to warm to 55° C. and stirred for overnight under nitrogen atmosphere. The mixture was allowed to cool down to 25° C. The reaction was quenched with sat.NH₄Cl (aq.) and extracted with Ethyl acetate (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1:2) to afford N-[(3S,4S)-4-[(5-bromo-1-{[2-(trimethylsilyl)ethoxy]methyl}pyrrolo[2,3-b]pyridin-6-yl)oxy]oxan-3-yl]-4-methylbenzenesulfonamide (3 g, 78.9%) as a white solid. LC-MS (ES, m/z) M+1: 596/598. ¹HNMR (300 MHz, Chloroform-d) δ 8.01 (s, 1H), 7.46 (d, J=8.1 Hz, 2H), 7.22 (d, J=3.6 Hz, 1H), 7.12 (d, J=8.0 Hz, 2H), 6.43 (d, J=3.6 Hz, 1H), 5.70 (d, J=10.8 Hz, 1H), 5.51 (d, J=10.8 Hz, 1H), 5.32 (s, 2H), 5.09 (td, J=7.9, 4.2 Hz, 1H), 4.23 (dd, J=11.6, 3.8 Hz, 1H), 3.98 (dt, J=10.6, 4.8 Hz, 1H), 3.68-3.49 (m, 3H), 3.42 (td, J=7.5, 3.8 Hz, 1H), 3.29 (dd, J=11.6, 7.5 Hz, 1H), 2.40 (s, 3H), 1.92 (dtd, J=13.1, 8.5, 4.1 Hz, 1H), 0.97 (ddd, J=8.5, 7.1, 3.1 Hz, 2H), −0.01 (s, 9H).

Synthesis of (3S,8S)-2-(4-methylbenzenesulfonyl)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene: Into a 250 mL round-bottom flask were added N-[(3S,4S)-4-[(5-bromo-1-{[2-(trimethylsilyl)ethoxy]methyl}pyrrolo[2,3-b]pyridin-6-yl)oxy]oxan-3-yl]-4-methylbenzenesulfonamide (3.0 g, 5.0 mmol, 1.0 eq), DMF (50 mL), CuI (575 mg, 3.0 mmol, 0.6 eq), K₂CO₃ (2.1 g, 15.2 mmol, 3.0 eq), 1,10-phenanthroline (545 mg, 3.0 mmol, 0.6 eq). The resulting mixture was stirred for overnight at 120° C. under nitrogen atmosphere. The mixture was allowed to cool down to 25° C. The resulting mixture was diluted with water (400 mL). The aqueous layer was extracted with Ethyl acetate (2×200 mL) and the organic layer was washed with brine (1×500 mL), the combined organic layer was dried over anhydrous Na₂SO₄. After filtration, the filtrate was concentrated under reduced pressure. The crude residue was purified by a flash column (silica gel, ethyl acetate/petroleum ether=2:3) to afford (3S,8S)-2-(4-methylbenzenesulfonyl)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (2 g, 77.1%) as a colorless oil. LC. MS (ES, m/z) M+1: 516. ¹HNMR (300 MHz, Chloroform-d) δ 8.45 (s, 1H), 7.28 (s, 1H), 7.23-7.17 (m, 2H), 7.11 (d, J=8.2 Hz, 2H), 6.57 (d, J=3.6 Hz, 1H), 5.55 (s, 2H), 4.73 (dd, J=10.1, 3.0 Hz, 1H), 4.07 (dd, J=12.0, 4.8 Hz, 1H), 3.73 (td, J=10.6, 4.4 Hz, 1H), 3.61-3.36 (m, 5H), 2.35 (s, 3H), 2.25-2.13 (m, 1H), 1.91 (qd, J=12.3, 5.0 Hz, 1H), 0.92 (dp, J=9.6, 7.1 Hz, 2H), −0.03 (s, 9H).

Synthesis of (3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene: Into a 40-mL vial purged and maintained with an inert atmosphere of nitrogen, was placed Na (536 mg, 23.3 mmol, 6.0 eq), naphthalene (3.0 g, 23.3 mmol, 6.0 eq), DME (15 mL). The mixture was stirred for 40 min at 25° C. until the formation of Na/naphthalene was complete. Another 100 mL 3-necked round-bottom flask purged and maintained with an inert atmosphere of nitrogen, was placed (3S,8S)-2-(4-methylbenzenesulfonyl)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (2.0 g, 3.9 mmol, 1.0 eq), THE (30 mL). This was followed by the addition of the above solution at −78° C. The resulting solution was stirred for 3 hours at 25° C. The reaction was then quenched by the addition of aqueous NH₄Cl (300 mL). The resulting solution was extracted with Ethyl acetate (3×100 mL) and the organic layers combined. The resulting mixture was washed with brine (1×300 mL). The mixture was dried over anhydrous sodium sulfate. After filtration, the filtrate was concentrated under reduced pressure. The crude residue was purified by a flash column (silica gel, ethyl acetate/petroleum ether=3:2) to afford (3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (1 g, 71.3%) as a yellow oil. LC-MS (ES, m/z) M+1: 362. ¹HNMR (300 MHz, Chloroform-d) δ 7.24 (s, 1H), 7.16 (d, J=3.6 Hz, 1H), 6.33 (d, J=3.6 Hz, 1H), 5.57 (s, 2H), 4.13 (p, J=5.3 Hz, 3H), 3.67-3.46 (m, 3H), 3.28 (d, J=6.6 Hz, 2H), 2.33-2.20 (m, 1H), 2.10-1.90 (m, 1H), 0.96-0.85 (m, 2H), −0.04 (s, 8H).

Synthesis of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide: Into a 40 mL vial were added 2-bromo-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide (114 mg, 0.1 mmol, 1.0 eq) and (3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (50 mg, 0.1 mmol, 1.0 eq), DMF (3 mL), Cs₂CO₃ (90 mg, 0.3 mmol, 2.0 eq), CuI (5.3 mg, 0.03 mmol, 0.2 eq), N,N′-diphenyl-ethanediamide (6.6 mg, 0.03 mmol, 0.2 eq). The resulting mixture was stirred for 6 hours at 100° C. under nitrogen atmosphere. The mixture was allowed to cool down to 25° C. The resulting mixture was diluted with water (30 mL). The resulting mixture was extracted with Ethyl acetate (3×20 mL). The combined organic layers were washed with brine (1×50 mL), dried over anhydrous Na₂SO₄. After filtration, the filtrate was concentrated under reduced pressure. The crude residue was purified by prep-TLC (dichloromethanel methanol=12:1) to afford 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide (60 mg, 39.2%) as a yellow solid. LC-MS (ES, m/z) M+1: 1105.

Synthesis of 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aS)-5a,8,9,9a-tetrahydro-6H. pyrano[4,3-b]pyrrolo[3′,2′:5,6]pyrido[3,2-e][1,4]oxazin-5(1H)-yl)benzamide: Into a 40 mL vial were added 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide (60 mg, 0.05 mmol, 1.0 eq), TBAF in THE (1.0 M, 5 mL), ethylenediamine (65 mg, 1.1 mmol, 20.0 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide (5 mg, 9.5%) as a yellow solid. LC-MS (ES, m/z) M+1: 975. ¹HNMR (300 MHz, DMSO-d₆) δ 10.86 (s, 1H), 8.30 (d, J=20.9 Hz, 2H), 7.76-6.22 (m, 12H), 5.97 (s, 1H), 4.60 (s, 1H), 4.25-3.63 (m, 5H), 3.28-2.66 (m, 11H), 2.41-1.36 (m, 18H), 1.26 (d, J=9.0 Hz, 11H).

### Example 59: Preparation of 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide

Synthesis of N-((3R,4R)-4-hydroxytetrahydro-2H-pyran-3-yl)-4-methylbenzenesulfonamide: A solution of (3R,4R)-3-aminotetrahydro-2H-pyran-4-ol (1.0 g, 8.5 mmol, 1.0 eq) in DCM (20 mL) was treated with TEA (2.1 g, 21.3 mmol, 2.5 eq) at 25° C. under nitrogen atmosphere followed by the addition of TsCl (1.6 g, 9.4 mmol, 1.1 eq) dropwise at 0° C. The resulting mixture was stirred for 4 hours at 25° C. under nitrogen atmosphere. The reaction was quenched by the addition of water (10 mL) at 0° C. The resulting mixture was extracted with CH₂Cl₂ (2×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford N-((3R,4R)-4-hydroxytetrahydro-2H-pyran-3-yl)-4-methylbenzenesulfonamide (1200 mg, 51.8%) as a white solid. LC-MS (ESI, m/z) M+1: 272. ¹HNMR (400 MHz, DMSO-d₆) δ 7.77-7.70 (m, 2H), 7.63 (d, J=6.9 Hz, 1H), 7.37 (d, J=8.0 Hz, 2H), 4.79 (d, J=4.5 Hz, 1H), 3.68 (dt, J=11.5, 4.4 Hz, 1H), 3.60 (dd, J=11.3, 4.2 Hz, 1H), 3.40 (dt, J=8.5, 3.8 Hz, 1H), 3.27 (ddd, J=11.7, 9.7, 2.9 Hz, 1H), 2.98 (dd, J=11.3, 8.3 Hz, 1H), 2.79 (qd, J=7.8, 4.1 Hz, 1H), 2.38 (s, 3H), 1.83 (dtd, J=13.3, 4.5, 2.9 Hz, 1H), 1.33 (dtd, J=13.4, 9.3, 4.2 Hz, 1H).

Synthesis of N-[(3R,4R)-4-[(5-bromo-1-{[2-(trimethylsilyl)ethoxy]methyl}pyrrolo[2,3-b]pyridin-6-yl)oxy]oxan-3-yl]-4-methylbenzenesulfonamide: Into a 100 mL round-bottom flask were added N-[(3R,4R)-4-hydroxyoxan-3-yl]-4-methylbenzenesulfonamide (1.7 g, 6.3 mmol, 1.0 eq), THE (15 mL). To the above solution was added NaH (766 mg, 19.1 mmol, 3.0 eq, 60%) at 0° C. The mixture was stirred for 30 min at 0° C. 5-bromo-6-fluoro-1-{[2-(trimethylsilyl)ethoxy]methyl}pyrrolo[2,3-b]pyridine (2.2 g, 6.4 mmol, 1.0 eq) was added and the mixture was allowed to warm to 55° C. and stirred for overnight under nitrogen atmosphere. The mixture was allowed to cool down to 25° C. The reaction was quenched with sat.NH₄Cl (aq.) and extracted with Ethyl acetate (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1:2) to afford N-[(3R,4R)-4-[(5-bromo-1-{[2-(trimethylsilyl)ethoxy]methyl}pyrrolo[2,3-b]pyridin-6-yl)oxy]oxan-3-yl]-4-methylbenzenesulfonamide (3 g, 78.9%) as a white solid. LC-MS (ES, m/z) M+1: 596/598. ¹HNMR (400 MHz, DMSO-d₆) δ 8.17 (s, 1H), 8.06 (d, J=7.1 Hz, 1H), 7.70-7.63 (m, 2H), 7.44 (d, J=3.6 Hz, 1H), 7.24 (d, J=8.1 Hz, 2H), 6.43 (d, J=3.5 Hz, 1H), 5.58 (d, J=10.8 Hz, 1H), 5.47 (d, J=10.8 Hz, 1H), 5.11 (td, J=7.2, 3.9 Hz, 1H), 3.88-3.73 (m, 2H), 3.50 (dt, J=15.9, 8.1 Hz, 3H), 3.28 (d, J=6.8 Hz, 1H), 2.31 (s, 3H), 2.28-2.18 (m, 1H), 1.60 (dd, J=13.2, 8.1 Hz, 1H), 0.89-0.75 (m, 2H), −0.10 (s, 9H).

Synthesis of (3R,8R)-2-(4-methylbenzenesulfonyl)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene: Into a 250 mL round-bottom flask were added N-[(3R,4R)-4-[(5-bromo-1-{[2-(trimethylsilyl)ethoxy]methyl}pyrrolo[2,3-b]pyridin-6-yl)oxy]oxan-3-yl]-4-methylbenzenesulfonamide (3.0 g, 5.0 mmol, 1.0 eq), DMF (50 mL), CuI (575 mg, 3.0 mmol, 0.6 eq), K₂CO₃ (2.1 g, 15.2 mmol, 3.0 eq), 1,10-phenanthroline (545 mg, 3.0 mmol, 0.6 eq). The resulting mixture was stirred for overnight at 120° C. under nitrogen atmosphere. The mixture was allowed to cool down to 25° C. The resulting mixture was diluted with water (400 mL). The aqueous layer was extracted with Ethyl acetate (2×200 mL) and the organic layer was washed with brine (1×500 mL), the combined organic layer was dried over anhydrous Na₂SO₄. After filtration, the filtrate was concentrated under reduced pressure. The crude residue was purified by a flash column (silica gel, ethyl acetate/petroleum ether=2:3) to afford (3R,8R)-2-(4-methylbenzenesulfonyl)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (2 g, 77.1%) as a colorless oil. LC. MS (ES, m/z) M+1: 516. ¹HNMR (400 MHz, DMSO-d₆) δ 8.29 (s, 1H), 7.52 (d, J=3.6 Hz, 1H), 7.26-7.13 (m, 4H), 6.59 (d, J=3.6 Hz, 1H), 5.76 (s, 1H), 5.44 (s, 2H), 4.39 (dd, J=9.6, 3.0 Hz, 1H), 3.94 (dd, J=11.7, 4.7 Hz, 1H), 3.90-3.76 (m, 1H), 3.57-3.34 (m, 5H), 2.31 (s, 3H), 2.08 (dd, J=12.1, 4.2 Hz, 1H), 1.76 (qd, J=12.0, 4.9 Hz, 1H), 0.90-0.73 (m, 2H), −0.09 (s, 9H).

Synthesis of (3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene: Into a 40-mL vial purged and maintained with an inert atmosphere of nitrogen, was placed Na (536 mg, 23.3 mmol, 6.0 eq), naphthalene (3.0 g, 23.3 mmol, 6.0 eq), DME (15 mL). The mixture was stirred for 40 min at 25° C. until the formation of Na/naphthalene was complete. Another 100 mL 3-necked round-bottom flask purged and maintained with an inert atmosphere of nitrogen, was placed (3R,8R)-2-(4-methylbenzenesulfonyl)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (2.0 g, 3.9 mmol, 1.0 eq), THE (30 mL). This was followed by the addition of the above solution at −78° C. The resulting solution was stirred for 3 hours at 25° C. The reaction was then quenched by the addition of aqueous NH₄Cl (300 mL). The resulting solution was extracted with Ethyl acetate (3×100 mL) and the organic layers combined. The resulting mixture was washed with brine (1×300 mL). The mixture was dried over anhydrous sodium sulfate. After filtration, the filtrate was concentrated under reduced pressure. The crude residue was purified by a flash column (silica gel, ethyl acetate/petroleum ether=3:2) to afford (3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (1 g, 71.3%) as a yellow oil. LC-MS (ES, m/z) M+1: 362. ¹HNMR (300 MHz, DMSO-d₆) δ 7.26 (d, J=3.5 Hz, 1H), 7.15 (s, 1H), 6.25 (d, J=3.5 Hz, 1H), 5.64-5.57 (m, 1H), 5.40 (s, 2H), 4.10-3.91 (m, 5H), 3.57-3.41 (m, 3H), 3.11 (t, J=10.5 Hz, 1H), 3.03-2.91 (m, 1H), 2.08 (dd, J=12.0, 4.4 Hz, 1H), 1.99 (s, 1H), 1.73 (qd, J=12.3, 5.0 Hz, 1H), 1.18 (t, J=7.1 Hz, 2H), 0.86-0.75 (m, 2H), −0.08 (s, 9H).

Synthesis of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide: Into a 40 mL vial were added 2-bromo-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide (114 mg, 0.1 mmol, 1.0 eq) and (3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (50 mg, 0.1 mmol, 1.0 eq), DMF (3 mL), Cs₂CO₃ (90 mg, 0.3 mmol, 2.0 eq), CuI (5.3 mg, 0.03 mmol, 0.2 eq), N,N′-diphenyl-ethanediamide (6.6 mg, 0.03 mmol, 0.2 eq). The resulting mixture was stirred for 6 hours at 100° C. under nitrogen atmosphere. The mixture was allowed to cool down to 25° C. The resulting mixture was diluted with water (30 mL). The resulting mixture was extracted with Ethyl acetate (3×20 mL). The combined organic layers were washed with brine (1×50 mL), dried over anhydrous Na₂SO₄. After filtration, the filtrate was concentrated under reduced pressure. The crude residue was purified by prep-TLC (dichloromethanel methanol=12:1) to afford 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide (60 mg, 39.2%) as a yellow solid. LC-MS (ES, m/z) M+1: 1105.

Synthesis of 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aR)-5a,8,9,9a-tetrahydro-6H. pyrano[4,3-b]pyrrolo[3′,2′:5,6]pyrido[3,2-e][1,4]oxazin-5(1H)-yl)benzamide: Into a 40 mL vial were added 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide (60 mg, 0.05 mmol, 1.0 eq), TBAF in THE (1.0 M, 5 mL), ethylenediamine (65 mg, 1.1 mmol, 20.0 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide (5 mg, 9.5%) as a yellow solid. LC-MS (ES, m/z) M+1: 975. ¹HNMR (300 MHz, DMSO-d₆) δ 10.86 (s, 1H), 8.30 (d, J=20.9 Hz, 2H), 7.76-6.22 (m, 12H), 4.60 (s, 2H), 4.25-3.63 (m, 5H), 3.28-2.66 (m, 11H), 2.41-1.36 (m, 18H), 1.26 (d, J=9.0 Hz, 11H).

### Example 60: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(4-fluoro-2-isopropylphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of (S)—N-[(2-bromo-4-fluorophenyl)methylidene]-2-methylpropane-2-sulfinamide: Into a 500 mL round-bottom flask were added 2-bromo-4-fluorobenzaldehyde (30 g, 147.8 mmol, 1.0 eq), THE (300 mL), (S)-2-methylpropane-2-sulfinamide (18.0 g, 148.5 mmol, 1.0 eq), Ti(OEt)₄ (68.0 g, 298.1 mmol, 2.0 eq). The resulting mixture was stirred for 2 hours at 55° C. The mixture was allowed to cool down to 25° C. The resulting mixture was concentrated under vacuum. The resulting solution was diluted with 2000/2000 mL of water and Ethyl acetate. The solids were filtered out. The organic layer was separated from the filtration. The mixture was dried over anhydrous sodium sulfate and concentrated under vacuum. This resulted in (S)—N-[(2-bromo-4-fluorophenyl)methylidene]-2-methylpropane-2-sulfinamide (45 g, 99.4%) as a yellow oil. ¹HNMR (400 MHz, Chloroform-d) δ 8.90 (d, J=3.6 Hz, 1H), 8.07 (ddt, J=8.8, 6.1, 2.0 Hz, 1H), 7.38 (ddd, J=8.1, 3.4, 1.7 Hz, 1H), 7.12 (td, J=8.6, 2.6 Hz, 1H), 1.28-1.24 (m, 9H).

Synthesis of (R)—N-[(1S)-1-(2-bromo-4-fluorophenyl)-3-(1,3-dioxan-2-yl)propyl]-2-methylpropane. 2-sulfinamide: Into a 500 mL 3-necked round-bottom flask purged and maintained with an inert atmosphere of nitrogen, was placed Mg (10.7 g, 440.2 mmol, 3.0 eq), I₂ (3.8 g, 15.0 mmol, 0.1 eq), THE (450 mL), the resulting solution was warmed to 65° C., followed by the addition of 2-(2-bromoethyl)-1,3-dioxane (11.5 g, 59.3 mmol, 0.4 eq) in THE (50 mL) at 65° C. After addition completed, the mixture was cooled to 25° C., another 2-(2-bromoethyl)-1,3-dioxane (57.4 g, 295.9 mmol, 2.0 eq) in THE (150 mL) was added. Another 3000 mL 3-necked round-bottom flask purged and maintained with an inert atmosphere of nitrogen, was placed a solution of (S)—N-[(2-bromo-4-fluorophenyl)methylidene]-2-methylpropane-2-sulfinamide (45.0 g, 147.0 mmol, 1.0 eq) in CH₂Cl₂ (100 mL). The above solution was added dropwise at −40° C. The resulting solution was stirred for 1 hour at −40° C. in a liquid nitrogen bath. The reaction was then quenched by the addition of aqueous NH₄Cl (500 mL). The resulting solution was extracted dichloromethane (3×500 mL) and the organic layers combined. The resulting mixture was washed with brine (1×1000 mL). The mixture was dried over anhydrous Na₂SO₄. After filtration, the filtrate was concentrated under reduced pressure. This resulted in (R)—N-[(1S)-1-(2-bromo-4-fluorophenyl)-3-(1,3-dioxan-2-yl)propyl]-2-methylpropane-2-sulfinamide (60 g, 87.0%) as yellow oil. ¹HNMR (300 MHz, Chloroform-d) δ 7.38 (dd, J=8.7, 6.0 Hz, 1H), 7.28 (ddd, J=8.2, 2.7, 0.9 Hz, 1H), 7.04 (td, J=8.3, 2.7 Hz, 1H), 4.51 (d, J=4.9 Hz, 1H), 3.88-3.68 (m, 5H), 2.08-1.83 (m, 3H), 1.71-1.50 (m, 3H), 1.22-1.17 (m, 9H).

Synthesis of (2S)-2-(2-bromo-4-fluorophenyl)pyrrolidine: Into a 2000 mL round-bottom flask, was placed (R)—N-[(1S)-1-(2-bromo-4-fluorophenyl)-3-(1,3-dioxan-2-yl)propyl]-2-methylpropane-2-sulfinamide (60.0 g, 142.1 mmol, 1.0 eq), TFA (300 mL), water (75 mL), the resulting solution was stirred for 1 hour at 25° C. Et₃SiH (50.0 g, 430.0 mmol, 3.0 eq) was added. The resulting solution was stirred overnight at 25° C. The resulting mixture was concentrated. The resulting solution was diluted with 1000 mL of water The resulting solution was extracted with MTBE (2×300 mL) and the organic layers combined. The resulting mixture was washed with HCl (2.0 M, 1×500 mL). The pH value of the aqueous was adjusted to 13 with NaOH (4.0 M). The resulting solution was extracted with dichloromethane (3×300 mL) and the organic layers combined. The mixture was dried over anhydrous Na₂SO₄. This resulted in (2S)-2-(2-bromo-4-fluorophenyl)pyrrolidine (15 g, 38.9%) as yellow oil. ¹HNMR (400 MHz, Methanol-d₄) δ 7.55 (dd, J=8.8, 6.1 Hz, 1H), 7.37 (dd, J=8.4, 2.6 Hz, 1H), 7.13 (td, J=8.5, 2.7 Hz, 1H), 4.47 (t, J=7.6 Hz, 1H), 3.26-3.11 (m, 1H), 3.01 (ddd, J=10.5, 8.1, 6.8 Hz, 1H), 2.37-2.26 (m, 1H), 1.98-1.82 (m, 2H), 1.57 (ddt, J=12.6, 9.0, 7.6 Hz, 1H).

Synthesis of tert-butyl (2S)-2-(2-bromo-4-fluorophenyl)pyrrolidine-1-carboxylate: Into a 250 mL round-bottom flask were added (2S)-2-(2-bromo-4-fluorophenyl)pyrrolidine (5.0 g, 20.5 mmol, 1.0 eq), CH₂Cl₂ (50 mL, 786.5 mmol, 38.4 eq), TEA (4.2 g, 41.5 mmol, 2.0 eq), DMAP (250 mg, 2.0 mmol, 0.1 eq), Boc₂O (5.4 g, 24.7 mmol, 1.2 eq). The resulting mixture was stirred for overnight at 25° C. The resulting mixture was diluted with CH₂Cl₂ (200 mL) and washed with HCl (0.5 M) and washed with aqueous NaHCO₃ (1×200 mL), dried over anhydrous Na₂SO₄. After filtration, the filtrate was concentrated under reduced pressure. The crude residue was purified by a flash column (silica gel, ethyl acetate/petroleum ether=5:1) to afford tert-butyl (2S)-2-(2-bromo-4-fluorophenyl)pyrrolidine-1-carboxylate (6 g, 92.2%) as a yellow oil. ¹HNMR (300 MHz, Chloroform-d) δ 7.34-7.28 (m, 1H), 7.19-7.07 (m, 1H), 7.00 (td, J=8.3, 2.5 Hz, 1H), 5.13 (d, J=21.4 Hz, 1H), 3.67 (s, 2H), 2.39 (s, 1H), 1.89 (t, J=6.4 Hz, 1H), 1.78 (d, J=12.8 Hz, 1H), 1.62 (s, 1H), 1.49 (s, 3H), 1.22 (s, 6H).

Synthesis of tert-butyl (2S)-2-[4-fluoro-2-(prop-1-en-2-yl)phenyl]pyrrolidine-1-carboxylate: Into a 250 mL round-bottom flask purged and maintained with an inert atmosphere of nitrogen, was placed tert-butyl (2S)-2-(2-bromo-4-fluorophenyl)pyrrolidine-1-carboxylate (6.0 g, 17.4 mmol, 1.0 eq), dioxane (90 mL), water (10 mL), 4,4,5,5-tetramethyl-2-(prop-1-en-2-yl)-1,3,2-dioxaborolane (7.0 g, 41.7 mmol, 2.4 eq), K₂CO₃ (7.2 g, 52.1 mmol, 3.0 eq), Pd(dppf)Cl₂ (1.4 g, 2.0 mmol, 0.1 eq). The resulting solution was stirred overnight at 80° C. The reaction mixture was cooled to 25° C. The resulting mixture was concentrated under vacuum. The crude residue was purified by a flash column (silica gel, ethyl acetate/petroleum ether=1:6). This resulted in tert-butyl (2S)-2-[4-fluoro-2-(prop-1-en-2-yl)phenyl]pyrrolidine-1-carboxylate (6 g, 87.4%) as yellow oil. ¹HNMR (400 MHz, Chloroform-d) δ 7.10 (dd, J=8.7, 5.7 Hz, 1H), 6.90 (td, J=8.5, 2.8 Hz, 1H), 6.80 (dd, J=9.4, 2.7 Hz, 1H), 5.25 (p, J=1.7 Hz, 1H), 4.89 (d, J=16.8 Hz, 2H), 3.68-3.57 (m, 3H), 2.27 (dt, J=15.5, 7.7 Hz, 1H), 1.95-1.78 (m, 2H), 1.70 (h, J=6.2, 5.8 Hz, 1H), 1.52-1.37 (m, 2H), 1.28 (s, 1H), 1.20 (s, 7H).

Synthesis of ert-butyl (2S)-2-(4-fluoro-2-isopropylphenyl)pyrrolidine-1-carboxylate: Into a 100 mL pressure tank reactor (10 atm), was placed tert-butyl (2S)-2-[4-fluoro-2-(prop-1-en-2-yl)phenyl]pyrrolidine-1-carboxylate (6.2 g, 15.2 mmol, 1.0 eq, 75%), EtOH (50 mL), Pd/C (215 mg, 0.2 mmol, 0.1 eq), to the above H₂ was introduced in. The resulting solution was stirred overnight at 50° C. The solids were filtered out. The resulting mixture was concentrated under vacuum. The crude residue was purified by a flash column (silica gel, ethyl acetate/petroleum ether=1:10). This resulted in tert-butyl (2S)-2-(4-fluoro-2-isopropylphenyl)pyrrolidine-1-carboxylate (3 g, 64.1%) as a pink oil. ¹HNMR (400 MHz, Chloroform-d) δ 7.06-7.00 (m, 1H), 6.96 (dd, J=10.7, 2.7 Hz, 1H), 6.81 (td, J=8.3, 2.7 Hz, 1H), 5.14 (d, J=53.6 Hz, 1H), 3.75-3.66 (m, 3H), 3.62 (s, 2H), 3.19 (p, J=7.2 Hz, 1H), 1.88 (td, J=7.3, 4.0 Hz, 3H), 1.52-1.45 (m, 2H), 1.29-1.17 (m, 15H).

Synthesis of (2S)-2-(4-fluoro-2-isopropylphenyl)pyrrolidine (HCl salt): Into a 100 mL round-bottom flask, was placed tert-butyl (2S)-2-(4-fluoro-2-isopropylphenyl)pyrrolidine-1-carboxylate (300 mg, 1.0 mmol, 1.0 eq), HCl (gas) in 1,4-dioxane (4 mol/L, 20 mL). The resulting solution was stirred for 1 hour at 25° C. The resulting mixture was concentrated under vacuum. This resulted in (2S)-2-(4-fluoro-2-isopropylphenyl)pyrrolidine (HCl salt) (200 mg, 98.9%) a yellow oil. ¹HNMR (400 MHz, Chloroform-d) δ 7.51 (dd, J=8.7, 6.1 Hz, 1H), 6.96 (dd, J=10.8, 2.8 Hz, 1H), 6.87 (td, J=8.3, 2.8 Hz, 1H), 4.43 (t, J=7.8 Hz, 1H), 3.28 (dddd, J=17.9, 10.2, 7.2, 5.2 Hz, 2H), 3.04 (ddd, J=10.1, 8.1, 6.8 Hz, 1H), 2.19 (dtd, J=12.5, 7.7, 4.9 Hz, 1H), 2.03-1.83 (m, 2H), 1.70-1.54 (m, 1H), 1.24 (dd, J=8.7, 6.8 Hz, 6H).

Synthesis of 4-{2-[(2S)-2-(4-fluoro-2-isopropylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 100 mL round-bottom flask were added (2S)-2-(4-fluoro-2-isopropylphenyl)pyrrolidine (34 mg, 0.2 mmol, 1.0 eq), CH₂Cl₂ (5 mL), N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (147 mg, 0.2 mmol, 1.0 eq), NaBH(OAc)₃ (70 mg, 0.3 mmol, 2.0 eq). The resulting solution was stirred for overnight at 40° C. The resulting mixture was concentrated under vacuum. The residue was purified by Prep-TLC (CH₂Cl₂ MeOH=10:1) to afford 4-{2-[(2S)-2-(4-fluoro-2-isopropylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (120 mg, 66.9%) as a yellow oil. LC-MS (ESI, m/z) M+1: 1094.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(4-fluoro-2-isopropylphenyl)pyrrolidin-1-yl]-7-azaspiro[3.5]nonan-7-yl}-N-{3-nitro-4-[(oxan-4-ylmethyl)amino]benzenesulfonyl}benzamide: Into a 100 mL round-bottom flask were added 4-{2-[(2S)-2-(4-fluoro-2-isopropylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (120 mg, 0.1 mmol, 1.0 eq), TBAF in THE (1.0 M, 10 mL), ethylenediamine (132 mg, 2.2 mmol, 20.0 eq). The resulting mixture was stirred for overnight at 70° C. The mixture was allowed to cool down to 25° C. The resulting mixture was diluted with water (100 mL). The resulting mixture was extracted with Ethyl acetate (3×50 mL). The combined organic layers were washed with brine (1×100 mL), dried over anhydrous Na₂SO₄. After filtration, the filtrate was concentrated under reduced pressure. The residue was purified by Prep-TLC (CH₂Cl₂/MeOH=10:1) and The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min) to afford 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(2S)-2-(4-fluoro-2-isopropylphenyl)pyrrolidin-1-yl]-7-azaspiro[3.5]nonan-7-yl}-N-{3-nitro-4-[(oxan-4-ylmethyl)amino]benzenesulfonyl}benzamide (29 mg, 27.4%) as a white solid. LC-MS (ESI, m/z) M+1: 964. ¹HNMR (400 MHz, DMSO-d₆) δ 11.20 (s, 1H), 8.71-8.17 (m, 2H), 7.77-6.52 (m, 12H), 6.07-5.97 (m, 1H), 4.86-3.38 (m, 11H), 3.14 (d, J=31.6 Hz, 7H), 2.01-1.09 (m, 25H).

### Example 61: Preparation of 4-{2-[4,4-dimethyl-2-(2-methylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen. 10-yl]-N-{3-nitro-4-[(oxan-4-ylmethyl)amino]benzenesulfonyl}benzamide

Synthesis of tert-butyl 3,3-dimethylpyrrolidine-1-carboxylate: Into a 250 mL round-bottom flask were added 3,3-dimethylpyrrolidine (1.0 g, 10.1 mmol, 1.0 eq), CH₂Cl₂ (20 mL), TEA (1.5 g, 14.8 mmol, 2.0 eq), DMAP (91 mg, 0.7 mmol, 0.1 eq), (Boc)₂O (1.9 g, 8.7 mmol, 1.2 eq). The resulting mixture was stirred for overnight at 25° C. The resulting mixture was diluted with CH₂Cl₂ (200 mL) and washed with HCl (0.5 M) and washed with aqueous NaHCO₃ (1×200 mL), dried over anhydrous Na₂SO₄. After filtration, the filtrate was concentrated under reduced pressure. The crude residue was purified by a flash column (silica gel, ethyl acetate/petroleum ether=5:1) to afford tert-butyl 3,3-dimethylpyrrolidine-1-carboxylate (1.0 g, 69.7%) as a yellow oil. ¹HNMR (300 MHz, Chloroform-d) δ 3.40 (dt, J=17.0, 7.1 Hz, 2H), 3.07 (d, J=18.6 Hz, 2H), 1.63 (td, J=7.2, 2.7 Hz, 2H), 1.47 (s, 9H), 1.07 (s, 6H).

Synthesis of tert-butyl 4,4-dimethyl-2-(2-methylphenyl)pyrrolidine-1-carboxylate: A solution of tert-butyl 3,3-dimethylpyrrolidine-1-carboxylate (1.4 g, 7.0 mmol, 3.0 eq), o-bromotoluene (397 mg, 2.3 mmol, 1.0 eq), 3-acetoxyquinuclidine (395 mg, 2.3 mmol, 1.0 eq), 4,7-dimethoxy-1,10-phenanthroline (56 mg, 0.2 mmol, 0.1 eq), Ir[df (CF₃)ppy]₂(dtbbpy)PF₆ (52 mg, 0.2 mmol, 0.02 eq), nickel(II) bromide trihydrate (64.0 mg, 0.2 mmol, 0.1 eq) in Dimethylsulfoxide:H₂O=9:1(50 mL) for 2 days at 25° C. under nitrogen atmosphere and blue LED. The resulting mixture was diluted with water (300 mL). The resulting mixture was extracted with Ethyl acetate (3×200 mL). The combined organic layers were washed with brine (1×300 mL), dried over anhydrous Na₂SO₄. After filtration, the filtrate was concentrated under reduced pressure. The crude residue was purified by a flash column (silica gel, ethyl acetate/petroleum ether=1:30) to afford tert-butyl 4,4-dimethyl-2-(2-methylphenyl)pyrrolidine-1-carboxylate (60 mg, 3.0%) as light yellow solid. LC-MS (ESI, m/z) M+1: 290.

Synthesis of 4,4-dimethyl-2-(2-methylphenyl)pyrrolidine: Into a 50 mL round-bottom flask, was placed tert-butyl 4,4-dimethyl-2-(2-methylphenyl)pyrrolidine-1-carboxylate (60 mg, 0.2 mmol, 1.0 eq), HCl (gas) in 1,4-dioxane (4.0 M, 5 mL). The resulting solution was stirred for 1 hour at 25° C. The resulting mixture was concentrated under vacuum. This resulted in 4,4-dimethyl-2-(2-methylphenyl)pyrrolidine (35 mg, 89.2%) a yellow oil. LC-MS (ESI, m/z) M+1: 190.

Synthesis of 4-{2-[4,4-dimethyl-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 50 mL round-bottom flask were added 4,4-dimethyl-2-(2-methylphenyl)pyrrolidine (30 mg, 0.2 mmol, 1.0 eq), CH₂Cl₂ (5 mL), N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (143 mg, 0.2 mmol, 1.0 eq), NaBH(OAc)₃ (67 mg, 0.3 mmol, 2.0 eq). The resulting solution was stirred for overnight at 40° C. The resulting mixture was concentrated under vacuum. The residue was purified by Prep-TLC (ethyl acetate/petroleum ether=1:1) to afford 4-{2-[4,4-dimethyl-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (30 mg, 17.6%) as a yellow oil. LC-MS (ESI, m/z) M+1: 1075.

Synthesis of 4-{2-[4,4-dimethyl-2-(2-methylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide: Into a 50 mL round-bottom flask were added 4-{2-[4,4-dimethyl-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (30 mg, 0.03 mmol, 1.0 eq), TBAF in THE (1.0 M, 10 mL), ethylenediamine (34 mg, 0.6 mmol, 20.0 eq). The resulting mixture was stirred for overnight at 70° C. The mixture was allowed to cool down to 25° C. The resulting mixture was diluted with water (100 mL). The resulting mixture was extracted with Ethyl acetate (3×50 mL). The combined organic layers were washed with brine (1×100 mL), dried over anhydrous Na₂SO₄. After filtration, the filtrate was concentrated under reduced pressure. The residue was purified by Prep-TLC (thyl acetate/petroleum ether=1:1) and The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 90% in 10 min) to afford 4-{2-[4,4-dimethyl-2-(2-methylphenyl)pyrrolidin-1-yl]-7-azaspiro[3.5]nonan-7-yl}-2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-N-{3-nitro-4-[(oxan-4-ylmethyl)amino]benzenesulfonyl}benzamide (4 mg, 13.3%) as a yellow solid. LC-MS (ESI, m/z) M+1: 945. ¹HNMR (300 MHz, DMSO-d₆) δ 11.28-11.07 (m, 1H), 8.34-8.27 (m, 2H), 7.75-6.40 (m, 12H), 6.06-5.93 (m, 1H), 4.78-3.42 (m, 9H), 3.29-2.69 (m, 6H), 2.42-0.99 (m, 29H). Example 62: Preparation of 4-(2-((S)-2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro. 1H-pyrano[3,4-b]pyrrolo[3′,2′:5,6]pyrido[3,2-e][1,4]oxazin-5(5aH)-yl)benzamide hydrochloride

Synthesis of N-((3S,4R)-4-hydroxytetrahydro-2H-pyran-3-yl)-4-methylbenzenesulfonamide: A solution of (3S,4R)-4-aminotetrahydro-2H-pyran-3-ol (3.5 g, 29.9 mmol, 1.0 eq) in CH₂Cl₂ (70 mL) was treated with TEA (7.6 g, 74.7 mmol, 2.5 eq) at 25° C. under nitrogen atmosphere followed by the addition of TsCl (5.7 g, 29.9 mmol, 1.0 eq) dropwise at 0° C. The resulting mixture was stirred for 4 hours at 25° C. under nitrogen atmosphere. The reaction was quenched by the addition of Water (30 mL) at 0° C. The resulting mixture was extracted with CH₂Cl₂ (2×20 mL). The combined organic layers were washed with brine (1×20 mL), dried over anhydrous Na₂SO₄. After filtration, the filtrate was concentrated under reduced pressure. The residue was purified by silica gel column chromatography, eluted with ethyl acetate/petroleum ether (1:1) to afford N-((3S,4R)-3-hydroxytetrahydro-2H-pyran-4-yl)-4-methylbenzenesulfonamide (4.0 g, 49.3%) as a white solid. LC. MS (ESI, m/z) M+1: 272. ¹HNMR (400 MHz, DMSO-d₆) δ 7.76-7.70 (m, 2H), 7.63 (d, J=6.9 Hz, 1H), 7.37 (d, J=8.1 Hz, 2H), 4.79 (d, J=4.7 Hz, 1H), 3.68 (dt, J=11.5, 4.4 Hz, 1H), 3.60 (dd, J=11.3, 4.1 Hz, 1H), 3.39 (tt, J=8.5, 4.3 Hz, 1H), 3.27 (ddd, J=11.4, 9.7, 2.9 Hz, 1H), 2.98 (dd, J=11.3, 8.3 Hz, 1H), 2.79 (qd, J=7.8, 4.1 Hz, 1H), 2.38 (s, 3H), 1.83 (dtd, J=13.3, 4.5, 3.0 Hz, 1H), 1.33 (dtd, J=13.4, 9.3, 4.2 Hz, 1H).

Synthesis of N-[(3S,4R)-3-[(5-bromo-1-{[2-(trimethylsilyl)ethoxy]methyl}pyrrolo[2,3-b]pyridin-6-yl)oxy]oxan-4-yl]-4-methylbenzenesulfonamide: Into a 100 mL round-bottom flask were added N-[(3S,4R)-3-hydroxyoxan-4-yl]-4-methylbenzenesulfonamide (4.0 g, 14.7 mmol, 1.0 eq), THE (80 mL). To the above solution was added NaH (1.8 g, 45.0 mmol, 3.0 eq 60%) at 0° C. The mixture was stirred for 30 min at 0° C. 5-bromo-6-fluoro-1-{[2-(trimethylsilyl)ethoxy]methyl}pyrrolo[2,3-b]pyridine (5.1 g, 14.8 mmol, 1.0 eq) was added and the mixture was allowed to warm to 50° C. and stirred for overnight under nitrogen atmosphere. The mixture was allowed to cool down to 25° C. The reaction was quenched with sat.NH₄Cl (aq.) and extracted with Ethyl acetate (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1:2) to afford N-[(3S,4R)-3-[(5-bromo-1-{[2-(trimethylsilyl)ethoxy]methyl}pyrrolo[2,3-b]pyridin-6-yl)oxy]oxan-4-yl]-4-methylbenzenesulfonamide (6.0 g, 67.1%) as a white solid. LC-MS (ES, m/z) M+1: 596/598. ¹HNMR (400 MHz, DMSO-d₆) δ 8.16 (s, 1H), 7.96 (d, J=7.2 Hz, 1H), 7.68-7.60 (m, 2H), 7.45 (d, J=3.6 Hz, 1H), 7.20 (d, J=8.1 Hz, 2H), 6.43 (d, J=3.6 Hz, 1H), 5.58 (d, J=10.8 Hz, 1H), 5.48 (d, J=10.9 Hz, 1H), 4.97 (td, J=7.5, 3.9 Hz, 1H), 3.76 (dt, J=11.6, 4.5 Hz, 1H), 3.59-3.38 (m, 4H), 2.27 (s, 3H), 1.96-1.83 (m, 1H), 1.55 (ddt, J=13.8, 9.2, 4.4 Hz, 1H), 0.92-0.74 (m, 2H), −0.09 (s, 9H).

Synthesis of (3S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 250 mL round-bottom flask were added N-[(3S,4R)-3-[(5-bromo-1-{[2-(trimethylsilyl)ethoxy]methyl}pyrrolo[2,3-b]pyridin-6-yl)oxy]oxan-4-yl]-4-methylbenzenesulfonamide (5.9 g, 9.9 mmol, 1.0 eq), DMF (60 mL), CuI (1.1 g, 5.8 mmol, 0.6 eq), K₂CO₃ (4.1 g, 29.7 mmol, 3.0 eq), 1,10-phenanthroline (1.1 g, 6.1 mmol, 0.6 eq). The resulting mixture was stirred for overnight at 120° C. under nitrogen atmosphere. The mixture was allowed to cool down to 25° C. The resulting mixture was diluted with water (400 mL). The aqueous layer was extracted with Ethyl acetate (2×200 mL) and the organic layer was washed with brine (1×500 mL), the combined organic layer was dried over anhydrous Na₂SO₄. After filtration, the filtrate was concentrated under reduced pressure. The crude residue was purified by a flash column (silica gel, ethyl acetate/petroleum ether=1:2) to afford (3S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (5.0 g, 92.2%) as a colorless oil. LC. MS (ES, m/z) M+1: 516. ¹HNMR (300 MHz, Chloroform-d) δ 8.45-8.42 (m, 2H), 7.31-7.24 (m, 2H), 7.23 (s, 1H), 7.17-7.09 (m, 2H), 6.56 (d, J=3.6 Hz, 1H), 5.55 (s, 2H), 4.25-4.17 (m, 1H), 4.06 (dd, J=11.9, 4.3 Hz, 1H), 3.63 (tt, J=9.0, 4.6 Hz, 1H), 3.60-3.41 (m, 4H), 3.31 (dd, J=10.8, 9.7 Hz, 1H), 2.69 (dt, J=14.0, 2.5 Hz, 1H), 2.36 (s, 3H), 2.06 (s, 5H), 1.03-0.81 (m, 2H), 0.04 (s, 7H).

Synthesis of (3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 40 mL vial purged and maintained with an inert atmosphere of nitrogen, was placed Na (1.5 g, 65.2 mmol, 7.0 eq), naphthalene (8.2 g, 64.0 mmol, 7.0 eq), DME (10 mL). The mixture was stirred for 40 min at 25° C. until the formation of Na/naphthalene was complete. Another 250 mL 3-necked round-bottom flask purged and maintained with an inert atmosphere of nitrogen, was placed (3S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (4.7 g, 9.1 mmol, 1.0 eq), THE (50 mL). This was followed by the addition of the above solution at −78° C. The resulting solution was stirred for 3 hours at 25° C. The reaction was then quenched by the addition of aqueous NH₄Cl (300 mL). The resulting solution was extracted with Ethyl acetate (3×200 mL) and the organic layers combined. The resulting mixture was washed with brine (1×300 mL). The mixture was dried over anhydrous sodium sulfate. After filtration, the filtrate was concentrated under reduced pressure. The crude residue was purified by a flash column (silica gel, ethyl acetate/petroleum ether=1:1) to afford (3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (2.1 g, 72.8%) as a yellow oil. LC-MS (ES, m/z) M+1: 362. ¹HNMR (400 MHz, Chloroform-d) δ 7.24 (s, 1H), 7.15 (d, J=3.5 Hz, 1H), 6.33 (d, J=3.5 Hz, 1H), 5.55 (s, 2H), 4.37-4.29 (m, 1H), 4.11-3.98 (m, 2H), 3.64-3.50 (m, 3H), 3.42 (t, J=10.6 Hz, 1H), 3.24 (ddd, J=12.7, 8.6, 4.5 Hz, 1H), 2.05-2.01 (m, 1H), 1.80 (td, J=12.2, 4.7 Hz, 1H), 0.98-0.84 (m, 2H), 0.05 (s, 9H).

Synthesis of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into a 40 mL vial were added (3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (50 mg, 0.1 mmol, 1.0 eq) and 2-bromo-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide (171 mg, 0.2 mmol, 1.5 eq), DMF (3 mL), Cs₂CO₃ (90 mg, 0.3 mmol, 2.0 eq), CuI (5 mg, 0.03 mmol, 0.2 eq), N,N′-diphenyl-ethanediamide (7 mg, 0.03 mmol, 0.2 eq). The resulting mixture was stirred for 6 hours at 100° C. under nitrogen atmosphere. The mixture was allowed to cool down to 25° C. The resulting mixture was diluted with water (30 mL). The resulting mixture was extracted with Ethyl acetate (3×20 mL). The combined organic layers were washed with brine (1×50 mL), dried over anhydrous Na₂SO₄. After filtration, the filtrate was concentrated under reduced pressure. The crude residue was purified by prep-TLC (dichloromethanel methanol=12:1) to afford 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (44 mg, 28.8%) as a yellow solid. LC-MS (ES, m/z) M+1: 1105.

Synthesis of 2-[(3S,8R)-2,5-dioxa-9,15,17-triazatetracyclo[8.7.0.0\hat{ }{3,8}.0\hat{ }{12,16}]heptadeca. 1(10),11,13,16-tetraen-9-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide hydrochloride: Into a 40 mL vial were added 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (44 mg, 0.04 mmol, 1.0 eq), TBAF in THE (1.0 M, 5 mL), ethylenediamine (48 mg, 0.8 mmol, 20.0 eq). The resulting mixture was stirred for 8 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). 2-[(3S,8R)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide hydrochloride (4 mg, 8.7%) as a yellow solid. LC-MS (ES, m/z) M+1: 975. ¹HNMR (300 MHz, DMSO-d₆) δ 12.75 (s, 1H), 11.24 (s, 1H), 10.44 (s, 1H), 8.46-6.24 (m, 12H), 6.08-5.97 (m, 1H), 4.82-3.72 (m, 8H), 3.09 (d, J=22.7 Hz, 6H), 2.43-1.41 (m, 13H), 1.34-1.21 (m, 19H).

### Example 63: Preparation of 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide hydrochloride

Synthesis of (S,E)-N-((3-bromothiophen-2-yl)methylene)-2-methylpropane-2-sulfinamide: Into a 500 mL round-bottom flask purged and maintained with an inert atmosphere of nitrogen. To a stirred mixture of 3-bromothiophene-2-carbaldehyde (3.0 g, 12.4 mmol, 1.0 eq) and Ti(OEt)₄ (6.8 g, 30 mmol, 2.0 eq) in THE (45 mL) was added (S)-2-methylpropane-2-sulfinamide (1.5 g, 12.4 mmol, 1.0 eq) in portions at 0° C. under N₂ atmosphere. The resulting mixture was stirred for 16 hours at 25° C. under N₂ atmosphere. The resulting solution was diluted with 200/200 mL of water and EtOAc. The solids were filtered out. The organic layer was separated from the filtration. The mixture was dried over anhydrous sodium sulfate and concentrated under vacuum. The resulting mixture was concentrated under reduced pressure. This resulted in (S,E)-N-((3-bromothiophen-2-yl)methylene)-2-methylpropane-2-sulfinamide (4.8 g, 82.3%) as a yellow oil. The crude product was used in the next step directly without further purification. LC-MS (ES, m/z) M+1: 294.

Synthesis of (R)—N—((S)-1-(3-bromothiophen-2-yl)-3-(1,3-dioxan-2-yl)propyl)-2-methylpropane-2-sulfinamide: Into a 500 mL round-bottom flask purged and maintained with an inert atmosphere of nitrogen. A solution of Mg (1.2 g, 49.0 mmol, 3.0 eq) in I₂ (400 mg, 1.6 mmol, 0.1 eq) was treated with THE (50 mL) for 0.5 hour at 25° C. under nitrogen atmosphere followed by the addition of 2-(2-bromoethyl)-1,3-dioxane (1.4 g, 8.0 mmol, 0.4 eq) dropwise at 25° C. The resulting mixture was stirred for 0.5 hour at 50° C. under N₂ atmosphere. To the above mixture was added 2-(2-bromoethyl)-1,3-dioxane (6.3 g, 32.0 mmol, 2.0 eq) dropwise over 0.5 hour at 25° C. The resulting mixture was stirred for additional 0.5 hour at 25° C. The resulting mixture was added (S,E)-N-((3-bromothiophen-2-yl)methylene)-2-methylpropane-2-sulfinamide (4.8 g, 16.3 mmol, 1.0 eq) in dichloromethane (50 mL) dropwise at −70° C. The resulting mixture was stirred for 1 hour at −70° C. under N₂ atmosphere. The resulting mixture was filtered, the filter cake was washed with dichloromethane (3×100 mL). The filtrate was concentrated under reduced pressure. This resulted in (R)—N—((S)-1-(3-bromothiophen-2-yl)-3-(1,3-dioxan-2-yl)propyl)-2-methylpropane-2-sulfinamide as a yellow oil. The crude product was used in the next step directly without further purification. LC-MS (ES, m/z) M+1: 410.

Synthesis of (S)-2-(3-bromothiophen-2-yl)pyrrolidine: Into a 250 mL round-bottom flask purged and maintained with an inert atmosphere of nitrogen. A solution of (R)—N—((S)-1-(3-bromothiophen-2-yl)-3-(1,3-dioxan-2-yl)propyl)-2-methylpropane-2-sulfinamide (7.2 g, 17.6 mmol, 1.0 eq) in water (16 mL) was treated with TFA (64 mL) for 3 hours at 25° C. under nitrogen atmosphere followed by the addition of triethylsilane (6.1 g, 52.6 mmol, 3.0 eq) dropwise at 25° C. The resulting mixture was stirred for 16 hours at 25° C. under N₂ atmosphere. The resulting mixture was diluted with 2-methoxy-2-methylpropane (100 mL). The mixture was basified to pH=13 with NaOH. The resulting mixture was washed with water (2×100 mL). The aqueous layer was extracted with dichloromethane (3×100 mL). The mixture was dried over anhydrous Na₂SO₄. After filtration, the filtrate was concentrated under reduced pressure. The residue was purified by silica gel column chromatography, eluted with dichloromethane/methanol (10:1) to afford (S)-2-(3-bromothiophen-2-yl)pyrrolidine (2.6 g, 63.8%) as a yellow oil. LC-MS (ES, m/z) M+1: 232.

Synthesis of tert-butyl (S)-2-(3-bromothiophen-2-yl)pyrrolidine-1-carboxylate: Into a 250 mL round-bottom flask purged and maintained with an inert atmosphere of nitrogen. A solution of (S)-2-(3-bromothiophen-2-yl)pyrrolidine (2.6 g, 11.2 mmol, 1.0 eq) in dichloromethane (30 mL) was treated with TEA (2.3 g, 22.4 mmol, 2.0 eq) for 0.5 hour at 25° C. under nitrogen atmosphere. This was followed by the addition of di-tert-butyl dicarbonate (2.9 g, 13.4 mmol, 1.2 eq) dropwise at 0° C. The resulting mixture was stirred for 16 hours at 25° C. under N₂ atmosphere. The resulting mixture was washed with HCl (1.0 M, 2×30 mL). The combined organic layers were washed with dichloromethane (3×50 mL), The mixture was dried over anhydrous Na₂SO₄. After filtration, the filtrate was concentrated under reduced pressure. After filtration, the filtrate was concentrated under reduced pressure. The residue was purified by silica gel column chromatography, eluted with ethyl acetate/petroleum ether (1:3) to afford tert-butyl (S)-2-(3-bromothiophen-2-yl)pyrrolidine-1-carboxylate (400 mg, 10.8%) as a yellow oil. LC-MS (ES, m/z) M+1: 332.

Synthesis of tert-butyl (S)-2-(3-(prop-1-en-2-yl)thiophen-2-yl)pyrrolidine-1-carboxylate: Into a 100 mL round-bottom flask purged and maintained with an inert atmosphere of nitrogen. A solution of tert-butyl (S)-2-(3-bromothiophen-2-yl)pyrrolidine-1-carboxylate (300 mg, 0.9 mmol, 1 eq) in 1,4-dioxane (9 mL) and water (1 mL) was treated with K₂CO₃ (375 mg, 2.7 mmol, 3.0 eq) and Pd(dppf)Cl₂ (66 mg, 0.1 mmol, 0.1 eq) under nitrogen atmosphere followed by the addition of 2-ethyl-4,4,5,5-tetramethyl-1,3,2-dioxaborolane (338 mg, 2.2 mmol, 2.4 eq) dropwise at 25° C. The resulting mixture was stirred for 16 hours at 80° C. under N₂ atmosphere. The resulting mixture was filtered, the filter cake was washed with dichloromethane (3×10 mL). The filtrate was concentrated under reduced pressure. The residue was purified by silica gel column chromatography, eluted with ethyl ‘acetate/petroleum ether (1:6) to afford tert-butyl (S)-2-(3-(prop-1-en-2-yl)thiophen-2-yl)pyrrolidine-1-carboxylate (200 mg, 75.5%) as a yellow oil. LC-MS (ESI, m/z) M+1: 294.

Synthesis of tert-butyl (S)-2-(3-isopropylthiophen-2-yl)pyrrolidine-1-carboxylate: Into a 50 mL round-bottom flask purged and maintained with an inert atmosphere of nitrogen. To a stirred mixture of tert-butyl (S)-2-(3-(prop-1-en-2-yl)thiophen-2-yl)pyrrolidine-1-carboxylate (200 mg, 1.8 mmol, 1.0 eq) in MeOH (8 mL) was added Pd/C (50 mg, 0.5 mmol, 0.8 eq) in portions at 25° C. under N₂ atmosphere. The resulting mixture was stirred for 16 hours at 50° C. under H₂ (10 atm) atmosphere. The resulting mixture was filtered, the filter cake was washed with MeOH (3×100 mL). The filtrate was concentrated under reduced pressure. The crude product was used in the next step directly without further purification. This resulted in tert-butyl (S)-2-(3-isopropylthiophen-2-yl)pyrrolidine-1-carboxylate (180 mg, 95.8%) as a yellow oil. LC-MS (ESI, m/z) M+1: 292.

Synthesis of (S)-2-(3-isopropylthiophen-2-yl)pyrrolidine: Into a 50 mL round-bottom flask purged and maintained with an inert atmosphere of nitrogen. A mixture of tert-butyl (S)-2-(3-isopropylthiophen-2-yl)pyrrolidine-1-carboxylate (180 mg, 0.7 mmol, 1.0 eq) in HCl(gas) in 1,4-dioxane (1 mL) was stirred for 1 hour at 25° C. under N₂ atmosphere. The resulting mixture was concentrated under reduced pressure. The crude product was used in the next step directly without further purification. This resulted in (S)-2-(3-isopropylthiophen-2-yl)pyrrolidine (10 mg, 76.4%) as a yellow oil. LC-MS (ESI, m/z) M+1: 192.

Synthesis of 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin. 5(1H)-yl)benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq) and (S)-2-(3-isopropylthiophen-2-yl)pyrrolidine (22 mg, 0.1 mmol, 1.0 eq), CH₂Cl₂ (3 mL), NaBH(OAc)₃ (47 mg, 0.2 mmol, 2.0 eq). The resulting mixture was stirred for overnight at 25° C. The reaction was then quenched by the addition of water (10 mL). The resulting solution was extracted with dichloromethane (2×20 mL). The resulting mixture was washed with brine (1×50 ml). The organic layers were combined and dried over anhydrous sodium sulfate and concentrated under vacuum. The residue was purified by Prep-TLC (dichloromethane/methanol=12:1) to afford 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (80 mg, 66.2%) as a yellow solid. LC-MS (ES, m/z) M+1: 1081.

Synthesis of 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide: Into a 100 mL round-bottom flask were added 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (80 mg, 0.07 mmol, 1.0 eq), TBAF in THE (1.0 M, 5 mL), ethylenediamine (88 mg, 1.5 mmol, 20.0 eq). The resulting mixture was stirred for 5 hours at 70° C. The mixture was allowed to cool down to 25° C. The resulting mixture was diluted with water (50 mL). The resulting mixture was extracted with ethyl acetate (3×30 mL). The combined organic layers were washed with brine (1×100 mL), dried over anhydrous Na₂SO₄. After filtration, the filtrate was concentrated under reduced pressure. The residue was purified by Prep-TLC (dichloromethane/methanol=11:1). The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide (8 mg, 11.4%) as a yellow solid. LC-MS (ES, m/z) M+1: 951. ¹HNMR (400 MHz, Chloroform-d) δ 12.43 (s, 1H), 8.58 (s, 2H), 8.46 (t, J=5.5 Hz, 1H), 8.07 (d, J=9.2 Hz, 1H), 7.83 (dd, J=9.1, 2.3 Hz, 1H), 7.23 (s, OH), 7.15-7.09 (m, 2H), 6.77 (s, 1H), 6.72 (d, J=9.2 Hz, 1H), 6.51 (d, J=2.5 Hz, 1H), 6.12 (dd, J=3.6, 1.9 Hz, 1H), 5.40-4.99 (m, 1H), 5.30-4.46 (m, 1H), 4.11 (d, J=9.9 Hz, 1H), 4.05 (dd, J=11.5, 5.0 Hz, 3H), 4.39-3.88 (m, 1H), 3.79 (dd, J=17.1, 7.9 Hz, 2H), 3.87-3.13 (m, 10H), 2.94 (d, J=90.4 Hz, 3H), 2.66-2.31 (m, 1H), 2.19 (s, 1H), 2.73-1.36 (m, 6H), 1.41 (dd, J=12.4, 4.5 Hz, 1H), 2.73-0.19 (m, 7H).

Synthesis of (S,E)-N-((3-bromothiophen-2-yl)methylene)-2-methylpropane-2-sulfinamide: Into a 500 mL round-bottom flask purged and maintained with an inert atmosphere of nitrogen. To a stirred mixture of 3-bromothiophene-2-carbaldehyde (3.0 g, 12.4 mmol, 1.0 eq) in THE (45 mL) was added (S)-2-methylpropane-2-sulfinamide (1.5 g, 12.4 mmol, 1.0 eq) in portions at 0° C. under N₂ atmosphere. The resulting mixture was stirred for 16 hours at 25° C. under N₂ atmosphere. The resulting mixture was concentrated under reduced pressure. This resulted in (S,E)-N-((3-bromothiophen-2-yl)methylene)-2-methylpropane-2-sulfinamide (4.8 g, 82.3%) as a yellow oil. The crude product was used in the next step directly without further purification. LC-MS (ES, m/z) M+1: 294.

Synthesis of (R)—N—((S)-1-(3-bromothiophen-2-yl)-3-(1,3-dioxan-2-yl)propyl)-2-methylpropane-2-sulfinamide: Into a 500 mL round-bottom flask purged and maintained with an inert atmosphere of nitrogen. A solution of Mg (1.2 g, 49.0 mmol, 3.0 eq) in I₂ (400 mg, 1.6 mmol, 0.1 eq) was treated with THE (50 mL) for 0.5 hour at 25° C. under nitrogen atmosphere followed by the addition of 2-(2-bromoethyl)-1,3-dioxane (1.4 g, 8.0 mmol, 0.4 eq) dropwise at 25° C. The resulting mixture was stirred for 0.5 hour at 50° C. under N₂ atmosphere. To the above mixture was added 2-(2-bromoethyl)-1,3-dioxane (6.3 g, 32.0 mmol, 2.0 eq) dropwise over 0.5 hour at 25° C. The resulting mixture was stirred for additional 0.5 hour at 25° C. The resulting mixture was added (S,E)-N-((3-bromothiophen-2-yl)methylene)-2-methylpropane-2-sulfinamide (4.8 g, 16.3 mmol, 1.0 eq) in dichloromethane (50 mL) dropwise at −70° C. The resulting mixture was stirred for 1 hour at −70° C. under N₂ atmosphere. The resulting mixture was filtered, the filter cake was washed with dichloromethane (3×100 mL). The filtrate was concentrated under reduced pressure. This resulted in (R)—N—((S)-1-(3-bromothiophen-2-yl)-3-(1,3-dioxan-2-yl)propyl)-2-methylpropane-2-sulfinamide as a yellow oil. The crude product was used in the next step directly without further purification. LC-MS (ES, m/z) M+1: 410.

Synthesis of (S)-2-(3-bromothiophen-2-yl)pyrrolidine: Into a 250 mL round-bottom flask purged and maintained with an inert atmosphere of nitrogen. A solution of (R)—N—((S)-1-(3-bromothiophen-2-yl)-3-(1,3-dioxan-2-yl)propyl)-2-methylpropane-2-sulfinamide (7.2 g, 17.6 mmol, 1.0 eq) in water (16 mL) was treated with TFA (64 mL) for 3 hours at 25° C. under nitrogen atmosphere followed by the addition of triethylsilane (6.1 g, 52.6 mmol, 3.0 eq) dropwise at 25° C. The resulting mixture was stirred for 16 hours at 25° C. under N₂ atmosphere. The resulting mixture was diluted with 2-methoxy-2-methylpropane (100 mL). The mixture was basified to pH 13 with NaOH. The resulting mixture was washed with water (2×100 mL). The aqueous layer was extracted with dichloromethane (3×100 mL). The mixture was dried over anhydrous Na₂SO₄. After filtration, the filtrate was concentrated under reduced pressure. The residue was purified by silica gel column chromatography, eluted with dichloromethane/methanol (10:1) to afford (S)-2-(3-bromothiophen-2-yl)pyrrolidine (2.6 g, 63.8%) as a yellow oil. LC-MS (ES, m/z) M+1: 232.

Synthesis of tert-butyl (S)-2-(3-bromothiophen-2-yl)pyrrolidine-1-carboxylate: Into a 250 mL round-bottom flask purged and maintained with an inert atmosphere of nitrogen. A solution of (S)-2-(3-bromothiophen-2-yl)pyrrolidine (2.6 g, 11.2 mmol, 1.0 eq) in dichloromethane (30 mL) was treated with TEA (2.3 g, 22.4 mmol, 2.0 eq) for 0.5 hour at 25° C. under nitrogen atmosphere. This was followed by the addition of di-tert-butyl dicarbonate (2.9 g, 13.4 mmol, 1.2 eq) dropwise at 0° C. The resulting mixture was stirred for 16 hours at 25° C. under N₂ atmosphere. The resulting mixture was washed with HCl (1.0 M, 2×30 mL). The combined organic layers were washed with dichloromethane (3×50 mL). The mixture was dried over anhydrous Na₂SO₄. After filtration, the filtrate was concentrated under reduced pressure. After filtration, the filtrate was concentrated under reduced pressure. The residue was purified by silica gel column chromatography, eluted with ethyl acetate/petroleum ether (1:3) to afford tert-butyl (S)-2-(3-bromothiophen-2-yl)pyrrolidine-1-carboxylate (400 mg, 10.8%) as a yellow oil. LC-MS (ES, m/z) M+1: 332.

Synthesis of tert-butyl (S)-2-(3-(prop-1-en-2-yl)thiophen-2-yl)pyrrolidine-1-carboxylate: Into a 100 mL round-bottom flask purged and maintained with an inert atmosphere of nitrogen. A solution of tert-butyl (S)-2-(3-bromothiophen-2-yl)pyrrolidine-1-carboxylate (300 mg, 0.9 mmol, 1 eq) in 1,4-dioxane (9 mL) and water (1 mL) was treated with K₂CO₃ (375 mg, 2.7 mmol, 3.0 eq) and Pd(dppf)Cl₂ (66 mg, 0.1 mmol, 0.1 eq) under nitrogen atmosphere followed by the addition of 2-ethyl-4,4,5,5-tetramethyl-1,3,2-dioxaborolane (338 mg, 2.2 mmol, 2.4 eq) dropwise at 25° C. The resulting mixture was stirred for 16 hours at 80° C. under N₂ atmosphere. The resulting mixture was filtered, the filter cake was washed with dichloromethane (3×10 mL). The filtrate was concentrated under reduced pressure. The residue was purified by silica gel column chromatography, eluted with ethyl acetate:petroleum ether (1:6) to afford tert-butyl (S)-2-(3-(prop-1-en-2-yl)thiophen-2-yl)pyrrolidine-1-carboxylate (200 mg, 75.5%) as a yellow oil. LC-MS (ESI, m/z) M+1: 294.

Synthesis of tert-butyl (S)-2-(3-isopropylthiophen-2-yl)pyrrolidine-1-carboxylate: Into a 50 mL round-bottom flask purged and maintained with an inert atmosphere of nitrogen. To a stirred mixture of tert-butyl (S)-2-(3-(prop-1-en-2-yl)thiophen-2-yl)pyrrolidine-1-carboxylate (200 mg, 1.8 mmol, 1.0 eq) in MeOH (8 mL) was added Pd/C (50 mg, 0.5 mmol, 0.8 eq) in portions at 25° C. under N₂ atmosphere. The resulting mixture was stirred for 16 hours at 50° C. under H₂ (10 atm) atmosphere. The resulting mixture was filtered, the filter cake was washed with MeOH (3×100 mL). The filtrate was concentrated under reduced pressure. The crude product was used in the next step directly without further purification. This resulted in tert-butyl (S)-2-(3-isopropylthiophen-2-yl)pyrrolidine-1-carboxylate (180 mg, 95.8%) as a yellow oil. LC-MS (ESI, m/z) M+1: 292.

Synthesis of (S)-2-(3-isopropylthiophen-2-yl)pyrrolidine: Into a 50 mL round-bottom flask purged and maintained with an inert atmosphere of nitrogen. A mixture of tert-butyl (S)-2-(3-isopropylthiophen-2-yl)pyrrolidine-1-carboxylate (180 mg, 0.7 mmol, 1.0 eq) in HCl(gas) in 1,4-dioxane (1 mL) was stirred for 1 hour at 25° C. under N₂ atmosphere. The resulting mixture was concentrated under reduced pressure. The crude product was used in the next step directly without further purification. This resulted in (S)-2-(3-isopropylthiophen-2-yl)pyrrolidine (10 mg, 76.4%) as a yellow oil. LC-MS (ESI, m/z) M+1: 192.

Synthesis of 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin. 5(1H)-yl)benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq) and (S)-2-(3-isopropylthiophen-2-yl)pyrrolidine (22 mg, 0.1 mmol, 1.0 eq), CH₂Cl₂ (3 mL), NaBH(OAc)₃ (47 mg, 0.2 mmol, 2.0 eq). The resulting mixture was stirred for overnight at 25° C. The reaction was then quenched by the addition of water (10 mL). The resulting solution was extracted with dichloromethane (2×20 mL). The resulting mixture was washed with brine (1×50 ml). The organic layers were combined and dried over anhydrous sodium sulfate and concentrated under vacuum. The residue was purified by Prep-TLC (dichloromethane/methanol=12:1) to afford 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (80 mg, 66.2%) as a yellow solid. LC-MS (ES, m/z) M+1: 1081.

Synthesis of 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide: Into a 100 mL round-bottom flask were added 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (80 mg, 0.07 mmol, 1.0 eq), TBAF in THE (1.0 M, 5 mL), ethylenediamine (88 mg, 1.5 mmol, 20.0 eq). The resulting mixture was stirred for 5 hours at 70° C. The mixture was allowed to cool down to 25° C. The resulting mixture was diluted with water (50 mL). The resulting mixture was extracted with ethyl acetate (3×30 mL). The combined organic layers were washed with brine (1×100 mL), dried over anhydrous Na₂SO₄. After filtration, the filtrate was concentrated under reduced pressure. The residue was purified by Prep-TLC (dichloromethane/methanol=11:1). The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in 4-(2-((S)-2-(3-isopropylthiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)benzamide (8 mg, 11.4%) as a yellow solid. LC-MS (ES, m/z) M+1: 951. ¹HNMR (400 MHz, Chloroform-d) δ 12.43 (s, 1H), 8.58 (s, 2H), 8.46 (t, J=5.5 Hz, 1H), 8.07 (d, J=9.2 Hz, 1H), 7.83 (dd, J=9.1, 2.3 Hz, 1H), 7.23 (s, OH), 7.15-7.09 (m, 2H), 6.77 (s, 1H), 6.72 (d, J=9.2 Hz, 1H), 6.51 (d, J=2.5 Hz, 1H), 6.12 (dd, J=3.6, 1.9 Hz, 1H), 5.40-4.99 (m, 1H), 5.30-4.46 (m, 1H), 4.11 (d, J=9.9 Hz, 1H), 4.05 (dd, J=11.5, 5.0 Hz, 3H), 4.39-3.88 (m, 1H), 3.79 (dd, J=17.1, 7.9 Hz, 2H), 3.87-3.13 (m, 10H), 2.94 (d, J=90.4 Hz, 3H), 2.66-2.31 (m, 1H), 2.19 (s, 1H), 2.73-1.36 (m, 6H), 1.41 (dd, J=12.4, 4.5 Hz, 1H), 2.73-0.19 (m, 7H).

### Example 64: 2-[(3R,8R)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of N-[(3R,4R)-3-hydroxyoxan-4-yl]-4-methylbenzenesulfonamide: A solution of (3R,4R)-4-aminooxan-3-ol (1.0 g, 8.5 mmol, 1.0 eq) in CH₂Cl₂ (10 mL) was treated with TEA (1.7 g, 17.1 mmol, 2.0 eq) at 25° C. under nitrogen atmosphere followed by the addition of p-toluenesulfonyl chloride (1.8 g, 9.4 mmol, 1.1 eq) dropwise at 0° C. The resulting mixture was stirred for 4 hours at 25° C. under nitrogen atmosphere. The reaction was quenched by the addition of water (10 mL) at 0° C. The resulting mixture was extracted with CH₂Cl₂ (2×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=2:1) to afford N-[(3R,4R)-3-hydroxyoxan-4-yl]-4-methylbenzenesulfonamide (1.0 g, 43.2%) as a white solid. LC-MS (ESI, m/z) M+1: 272. ¹HNMR (300 MHz, Chloroform-d) δ 7.84-7.66 (m, 2H), 7.37-7.30 (m, 2H), 5.09 (d, J=8.9 Hz, 1H), 3.86 (dt, J=12.2, 1.8 Hz, 1H), 3.52 (s, 1H), 3.47-3.32 (m, 2H), 2.45 (s, 3H), 1.87-1.75 (m, 2H), 1.70-1.60 (m, 1H).

Synthesis of N-[(3R,4R)-3-[(5-bromo-1-{[2-(trimethylsilyl)ethoxy]methyl}pyrrolo[2,3-b]pyridin-6-yl)oxy]oxan-4-yl]-4-methylbenzenesulfonamide: Into a 100 mL round-bottom flask were added N-[(3R,4R)-3-hydroxyoxan-4-yl]-4-methylbenzenesulfonamide (1.0 g, 3.7 mmol, 1.0 eq), THE (10 mL). To the above solution was added NaH (443 mg, 11.1 mmol, 3.0 eq, 60%) at 0° C. The mixture was stirred for 30 minutes at 0° C. 5-bromo-6-fluoro-1-{[2-(trimethylsilyl)ethoxy]methyl}pyrrolo[2,3-b]pyridine (1.3 g, 3.6 mmol, 1.0 eq) was added and the mixture was allowed to warm to 50° C. and stirred for overnight under nitrogen atmosphere. The mixture was allowed to cool down to 25° C. The reaction was quenched with sat.NH₄Cl (aq.) and extracted with Ethyl acetate (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1:2) to afford N-[(3R,4R)-3-[(5-bromo-1-{[2-(trimethylsilyl)ethoxy]methyl}pyrrolo[2,3-b]pyridin-6-yl)oxy]oxan-4-yl]-4-methylbenzenesulfonamide (1.0 g, 45.5%) as a white solid. LC-MS (ES, m/z) M+1: 596/598. ¹HNMR (400 MHz, Chloroform-d) δ 8.01 (s, 1H), 7.68-7.59 (m, 2H), 7.16 (d, J=3.5 Hz, 1H), 6.94-6.87 (m, 2H), 6.42 (d, J=3.5 Hz, 1H), 5.50-5.30 (m, 2H), 5.14 (d, J=8.5 Hz, 1H), 4.77 (d, J=3.6 Hz, 1H), 4.28 (dd, J=12.7, 3.4 Hz, 1H), 4.01 (dt, J=11.9, 3.9 Hz, 1H), 3.81 (ddt, J=1.8, 8.1, 4.0 Hz, 1H), 3.63-3.39 (m, 4H), 2.20 (dtd, J=13.2, 10.7, 4.3 Hz, 1H), 2.05 (s, 3H), 1.86-1.72 (m, 1H), 0.88 (ddd, J=82, 7.3, 2.2 Hz, 2H), −0.04 (s, 9H).

Synthesis of (3R,8R)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 250 mL round-bottom flask were added N-[(3R,4R)-3-[(5-bromo-1-{[2-(trimethylsilyl)ethoxy]methyl}pyrrolo[2,3-b]pyridin-6-yl)oxy]oxan-4-yl]-4-methylbenzenesulfonamide (1.0 g, 1.7 mmol, 1 eq), DMF (10 mL), CuI (190 mg, 1.0 mmol, 0.6 eq), K₂CO₃ (463 mg, 3.3 mmol, 2.0 eq), 1,10-phenanthroline (181 mg, 1 mmol, 0.6 eq). The resulting mixture was stirred for overnight at 120° C. under nitrogen atmosphere. The mixture was allowed to cool down to 25° C. The resulting mixture was diluted with water (400 mL). The aqueous layer was extracted with Ethyl acetate (2×200 mL) and the organic layer was washed with brine (1×500 mL), the combined organic layer was dried over anhydrous Na₂SO₄. After filtration, the filtrate was concentrated under reduced pressure. The crude residue was purified by a flash column (silica gel, ethyl acetate/petroleum ether=2:3) to afford (3R,8R)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (600 mg, 69.4%) as a colorless oil. LC-MS (ES, m/z) M+1: 516. ¹NMR (400 MHz, Chloroform-d) δ 8.46 (s, 1H), 7.44-7.39 (m, 2H), 7.27 (d, J=3.6 Hz, 1H), 7.19 (d, J=8.0 Hz, 2H), 6.54 (d, J=3.6 Hz, 1H), 5.65-5.51 (m, 2H), 4.40 (ddd, J=9.7, 6.3, 3.1 Hz, 1H), 4.15-4.11 (m, 1H), 4.06-3.96 (m, 1H), 3.61-3.53 (m, 3H), 3.46 (dd, J=13.0, 1.3 Hz, 1H), 3.40-3.30 (m, 1H), 2.38 (s, 3H), 1.73 (dd, J=9.4, 3.8 Hz, 2H), 0.93 (ddd, J=8.5, 7.3, 3.8 Hz, 2H), −0.04 (s, 9H).

Synthesis of (3R,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 40-mL vial purged and maintained with an inert atmosphere of nitrogen, was placed Na (188 mg, 8.2 mmol, 7.0 eq), naphthalene (1.0 g, 8.2 mmol, 7.0 eq), DME (10 mL). The mixture was stirred for 40 minutes at 25° C. until the formation of Na/naphthalene was complete. Another 100 mL 3-necked round-bottom flask purged and maintained with an inert atmosphere of nitrogen, was placed (3R,8R)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (600 mg, 1.2 mmol, 1.0 eq), THF (20 mL) This was followed by the addition of the above solution at −78° C. The resulting solution was stirred for 3 hours at 25° C. The reaction was then quenched by the addition of aqueous NH₄Cl (300 mL). The resulting solution was extracted with Ethyl acetate (3×100 mL) and the organic layers combined. The resulting mixture was washed with brine (1×300 mL). The mixture was dried over anhydrous sodium sulfate. After filtration, the filtrate was concentrated under reduced pressure. The crude residue was purified by a flash column (silica gel, ethyl acetate/petroleum ether=3:2) to afford (3R,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (300 mg, 71.3%) as a yellow oil. LC-MS (ES, m/z) M+1: 362. ¹HNMR (400 MHz, Chloroform-d) δ 7.16 (s, 1H), 7.13 (d, J=3.6 Hz, 1H), 6.30 (d, J=3.5 Hz, 1H), 5.55 (d, J=1.0 Hz, 2H), 4.44 (dt, J=5.5, 2.8 Hz, 1H), 3.97 (ddd, J=11.7, 5.9, 3.8 Hz, 1H), 3.78 (dd, J=12.0, 2.9 Hz, 1H), 3.66-3.50 (m, 5H), 1.96 (tdd, J=12.2, 9.3, 5.2 Hz, 1H), 1.86-1.72 (m, 1H), 0.92-0.86 (m, 2H), −0.05 (s, 9H).

Synthesis of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into a 40 mL vial were added (3R,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (90 mg, 0.2 mmol, 1.0 eq), DMF (3 mL), Cs₂CO₃ (162 mg, 0.5 mmol, 2.0 eq), CuI (9 mg, 0.05 mmol, 0.2 eq), N,N′-diphenyl-ethanediamide (12 mg, 0.05 mmol, 0.2 eq). The resulting mixture was stirred for 6 hours at 100° C. under nitrogen atmosphere. The mixture was allowed to cool down to 25° C. The resulting mixture was diluted with water (30 mL). The resulting mixture was extracted with Ethyl acetate (3×20 mL). The combined organic layers were washed with brine (1×50 mL), dried over anhydrous Na₂SO₄. After filtration, the filtrate was concentrated under reduced pressure. The crude residue was purified by prep-TLC (dichloromethanel methanol=12:1) to afford 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (200 mg, 72.7%) as a yellow solid. as a yellow solid. LC-MS (ES, m/z) M+1: 1105.

Synthesis of 2-[(3R,8R)-2,5-dioxa-9,15,17-triazatetracyclo[8.7.0.0\hat{ }{3,8}.0\hat{ }{12,16}]heptadeca. 1(10),11,13,16-tetraen-9-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide: Into a 40 mL vial were added 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (200 mg, 0.2 mmol, 1.0 eq), TBAF in THE (1.0 M, 4 mL), ethylenediamine (220 mg, 3.6 mmol, 20.2 eq). The resulting mixture was stirred for 6 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in 2-[(3R,8R)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide (33 mg, 18.7%) as a yellow solid. LC-MS (ES, m/z) M+1: 975. ¹HNMR (300 MHz, DMSO-d₆) δ 10.90 (s, 1H), 8.36 (s, 2H), 7.70-6.25 (m, 12H), 5.98 (s, 1H), 4.82-3.46 (m, 8H), 3.29-2.93 (m, 6H), 2.35-1.72 (m, 7H), 1.75-1.41 (m, 8H), 1.38-1.19 (m, 17H).

### Example 65: Preparation of N-((3-nitro-4-(((tetrahydro-2H-pyran-4-yl)methyl)amino)phenyl)sulfonyl)-2-((5aR,8aS)-5a,6,8,8a-tetrahydrofuro[3,4-b]pyrrolo[3′,2′:5,6]pyrido[3,2-e][1,4]oxazin-5(1H)-yl)-4-(2-((R)-3-(o-tolyl)-1,4-oxazepan-4-yl)-7-azaspiro[3.5]nonan-7-yl)benzamide

Synthesis of 2-amino-2-(2-methylphenyl)ethanol: To a stirred solution of amino (2-methylphenyl)acetic acid (6.0 g, 36.3 mmol, 1.0 eq) in THE (100 mL) were added BH₃-THF (148 mL, 148.0 mmol, 4.0 eq) dropwise at 25° C. under nitrogen atmosphere. The resulting mixture was stirred for overnight at 25° C. The reaction was quenched with HCl (1.0 M). The resulting mixture was concentrated under vacuum. The residue was basified to pH 12 with NaOH (4.0 M). The resulting mixture was extracted with CH₂Cl₂ (3×100 mL). The combined organic layers were washed with brine (1×300 mL), dried over anhydrous Na₂SO₄. After filtration, the filtrate was concentrated under reduced pressure. This resulted in 2-amino-2-(2-methylphenyl)ethanol (4.5 g, 81.9%) as a white solid. ¹HNMR (300 MHz, DMSO-d₆) δ 7.51-7.45 (m, 1H), 7.19-7.11 (m, 1H), 7.14-7.05 (m, 2H), 4.77 (s, 1H), 4.11 (dd, J=8.3, 4.2 Hz, 1H), 3.44 (dd, J=10.5, 4.3 Hz, 1H), 3.21 (dd, J=10.5, 8.3 Hz, 1H), 2.31 (s, 3H).

Synthesis of tert-butyl N-[2-hydroxy-1-(2-methylphenyl)ethyl]carbamate: Into a 250 mL round-bottom flask were added 2-amino-2-(2-methylphenyl)ethanol (4.3 g, 28.1 mmol, 1.0 eq), CH₂Cl₂ (50 mL), TEA (5.7 g, 56.3 mmol, 2.0 eq), DMAP (344 mg, 2.8 mmol, 0.1 eq), Boc₂O (7.4 g, 33.9 mmol, 1.2 eq). The resulting mixture was stirred for overnight at 25° C. The resulting mixture was diluted with HCl (0.2 M). The resulting mixture was extracted with CH₂Cl₂ (3×50 mL). The combined organic layers were washed with brine (1×300 mL), dried over anhydrous Na₂SO₄. After filtration, the filtrate was concentrated under reduced pressure. The crude product was re-crystallized from ethyl acetate/petroleum ether (1:2) to afford tert-butyl N-[2-hydroxy-1-(2-methylphenyl)ethyl]carbamate (5 g, 69.4%) as a white solid. ¹HNMR (300 MHz, DMSO-d₆) δ 7.34-7.06 (m, 5H), 4.80 (t, J=6.2 Hz, 2H), 3.41 (t, J=6.3 Hz, 2H), 2.33 (s, 3H), 1.36 (s, 9H).

Synthesis of tert-butyl 3-{2-[(tert-butoxycarbonyl)amino]-2-(2-methylphenyl)ethoxy}propanoate: Into a 40 mL vial were added tert-butyl N-[2-hydroxy-1-(2-methylphenyl)ethyl]carbamate (370 mg, 1.5 mmol, 1.0 eq), t-BuOH (5 mL), tert-butyl prop-2-enoate (3.8 g, 29.6 mmol, 20.1 eq), Cs₂CO₃ (479 mg, 1.5 mmol, 1.0 eq). The resulting mixture was stirred for overnight at 25° C. The reaction was diluted with water (300 mL). The resulting solution was extracted with ethyl acetate (3×50 mL) and the organic layers combined. The resulting mixture was washed with brine (1×300 mL). The mixture was dried over anhydrous Na₂SO₄. After filtration, the filtrate was concentrated under reduced pressure. The crude residue was purified by a flash column (silica gel, ethyl acetate/petroleum ether=1:10) to afford tert-butyl 3-{2-[(tert-butoxycarbonyl)amino]-2-(2-methylphenyl)ethoxy}propanoate (530 mg, 94.8%) as a colorless oil. LC-MS (ES, m/z) M+1: 380.

Synthesis of 3-[2-amino-2-(2-methylphenyl)ethoxy]propanoic acid hydrochloride: A solution tert-butyl 3-{2-[(tert-butoxycarbonyl)amino]-2-(2-methylphenyl)ethoxy}propanoate (500 mg, 1.3 mmol, 1.0 eq) in HCl (gas) in 1,4-dioxane (4.0 M, 5 mL) was stirred for 3 h at 25° C. The resulting mixture was concentrated under vacuum. This resulted in 3-[2-amino-2-(2-methylphenyl)ethoxy]propanoic acid hydrochloride (340 mg, 99.4%) as a white solid. LC-MS (ES, m/z) M+1: 224.

Synthesis of 3-(2-methylphenyl)-1,4-oxazepan-5-one: Into a 40 mL vial were added 3-[2-amino-2-(2-methylphenyl)ethoxy]propanoic acid hydrochloride (340 mg, 1.3 mmol, 1.0 eq), CH₂Cl₂ (5 mL), EDCI (301 mg, 1.6 mmol, 1.2 eq), HOBT (212 mg, 1.6 mmol, 1.2 eq), TEA (265 mg, 2.6 mmol, 2.0 eq). The resulting mixture was stirred for overnight at 25° C. The resulting mixture was concentrated under vacuum. The crude residue was purified by a flash column (silica gel, dichloromethane/methanol=10:1) to afford 3-(2-methylphenyl)-1,4-oxazepan-5-one (200 mg, 74.4%) as a white solid. LC-MS (ES, m/z) M+1: 206. ¹HNMR (300 MHz, DMSO-d₆) δ 7.43 (s, 1H), 7.35-7.26 (m, 1H), 7.20 (d, J=2.5 Hz, 3H), 4.90 (dt, J=7.4, 2.9 Hz, 1H), 3.91 (ddd, J=12.4, 5.5, 2.8 Hz, 1H), 3.80-3.66 (m, 2H), 3.70-3.58 (m, 1H), 3.04 (ddd, J=15.5, 10.6, 2.8 Hz, 1H), 2.45-2.37 (m, 1H), 2.35 (s, 3H).

Synthesis of (3S)-3-(2-methylphenyl)-1,4-oxazepane and (3R)-3-(2-methylphenyl)-1,4-oxazepane: To a stirred solution of 3-(2-methylphenyl)-1,4-oxazepan-5-one (200 mg, 1.0 mmol, 1.0 eq) in THE (10 mL) was added LAH (111 mg, 3.0 mmol, 3.00 eq) in portions at 0° C. under nitrogen atmosphere. The resulting mixture was stirred for overnight at 25° C. The reaction was quenched with Na₂SO₄.10H₂O at 0° C. The resulting mixture was filtered. The filtrate was concentrated under reduced pressure. The crude residue was purified by a flash column (silica gel, dichloromethane/methanol=10:1). The crude product was purified by Chiral-Prep-HPLC with the following conditions. This resulted in (3S)-3-(2-methylphenyl)-1,4-oxazepane (43 mg, 23.1%) as a light yellow oil and (3R)-3-(2-methylphenyl)-1,4-oxazepane (43 mg, 23.1%) as a light yellow oil. 7A, T_{R}=9.5 min in CHIRAL-SFC, Column: CHIRALPAK IE, 3*25 cm, 5 μm; Mobile Phase A: Hex (0.1%2M NH₃-MeOH), Mobile Phase B: EtOH; Flow rate: 35 mL/min; Gradient: 10% B up to 10% B in 12 minutes, Wave Length: 254 nm. 7B, T_{R}=10.5 min in CHIRAL-SFC, Column: CHIRALPAK IE, 3*25 cm, 5 μm; Mobile Phase A: Hex (0.1% 2M NH₃-MeOH), Mobile Phase B: EtOH; Flow rate: 35 mL/min; Gradient: 10% B up to 10% B in 12 minutes, Wave Length: 254 nm. LC-MS (ES, m/z) M+1: 192 (peak 1). ¹HNMR (300 MHz, DMSO-d₆) δ 7.52-7.44 (m, 1H), 7.28-7.12 (m, 3H), 4.17 (dd, J=9.7, 3.2 Hz, 1H), 4.04 (dt, J=12.2, 6.1 Hz, 1H), 3.97-3.81 (m, 2H), 3.48 (dd, J=12.3, 9.6 Hz, 1H), 3.27 (dt, J=13.4, 5.0 Hz, 1H), 3.03 (dt, J=13.3, 7.2 Hz, 1H), 2.40 (s, 3H), 2.03 (ddt, J=12.8, 6.5, 3.5 Hz, 3H). LC-MS (ES, m/z) M+1: 192 (peak 1). ¹HNMR (300 MHz, DMSO-d₆) δ 7.52-7.44 (m, 1H), 7.28-7.12 (m, 3H), 4.17 (dd, J=9.7, 3.2 Hz, 1H), 4.04 (dt, J=12.2, 6.1 Hz, 1H), 3.97-3.81 (m, 2H), 3.48 (dd, J=12.3, 9.6 Hz, 1H), 3.27 (dt, J=13.4, 5.0 Hz, 1H), 3.03 (dt, J=13.3, 7.2 Hz, 1H), 2.40 (s, 3H), 2.03 (ddt, J=12.8, 6.5, 3.5 Hz, 3H).

Synthesis of 4-{2-[(3R)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq) and (3R)-3-(2-methylphenyl)-1,4-oxazepane (21 mg, 0.1 mmol, 1.0 eq), CH₂Cl₂ (3 mL), NaBH(OAc)₃ (47 mg, 0.2 mmol, 2.0 eq). The resulting mixture was stirred for 5 days at 45° C. The mixture was allowed to cool down to 25° C. The resulting mixture was concentrated under vacuum. The crude residue was purified by a flash column (silica gel, dichloromethane/methanol=12:1) to afford 4-{2-[(3R)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 83.7%) as a yellow solid. LC-MS (ES, m/z) M+1: 1077.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(3R)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}benzamide: Into a 100 mL round-bottom flask were added 4-{2-[(3R)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.09 mmol, 1.0 eq), TBAF in THE (1.0 M, 5 mL), ethylenediamine (112 mg, 1.8 mmol, 20.0 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(3R)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}benzamide (28 mg, 31.8%) as a yellow solid. LC-MS (ES, m/z) M+1: 947. ¹HNMR (300 MHz, DMSO-d₆) δ 11.01 (s, 1H), 8.44-8.15 (m, 2H), 7.64-7.32 (m, 3H), 7.19-6.96 (m, 4H), 6.79-6.15 (m, 5H), 6.11-5.92 (m, 1H), 4.60 (dq, J=89.0, 8.3 Hz, 1H), 4.10 (dt, J=14.0, 7.3 Hz, 1H), 3.93-3.79 (m, 4H), 3.78-3.34 (m, 8H), 3.29-2.78 (m, 10H), 2.29 (s, 3H), 1.87 (s, 3H), 1.76-1.57 (m, 4H), 1.49 (d, J=7.8 Hz, 3H), 1.40-1.18 (m, 5H).

### Example 66: Preparation of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(3S)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}benzamide

Synthesis of 4-{2-[(3S)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}. 13,16-dioxa-2,4,10-triazatetracyclo[7.7.0.0\hat{ }{3,7}.0\hat{ }{11,15}]hexadeca-1(9),2,5,7-tetraen-10-yl]benzamide: Into a 20 mL vial were added N-{3-nitro-4-[(oxan-4-ylmethyl)amino]benzenesulfonyl}-4-{2-oxo-7-azaspiro[3.5]nonan-7-yl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (100 mg, 0.1 mmol, 1.0 eq) and (3S)-3-(2-methylphenyl)-1,4-oxazepane (21 mg, 0.1 mmol, 1.0 eq), CH₂Cl₂ (3 mL), NaBH(OAc)₃ (47 mg, 0.2 mmol, 2.0 eq). The resulting mixture was stirred for 5 days at 45° C. The mixture was allowed to cool down to 25° C. The resulting mixture was concentrated under vacuum. The crude residue was purified by a flash column (silica gel, dichloromethane/methanol=12:1) to afford 4-{2-[(3S)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (90 mg, 75.4%) as a yellow solid. LC-MS (ES, m/z) M+1: 1077.

Synthesis of 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(3S)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}benzamide: Into a 100 mL round-bottom flask were added 4-{2-[(3S)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}-2-[(11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraen-10-yl]benzamide (90 mg, 0.08 mmol, 1.0 eq), TBAF in THE (1.0 M, 5 mL), ethylenediamine (100 mg, 1.6 mmol, 20.0 eq). The resulting mixture was stirred for 3 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. After filtration, the filtrate was concentrated under reduced pressure. The crude product was purified by Prep-HPLC with the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. This resulted in 2-[(11R,15S)-13,16-dioxa-2,4,10-triazatetracyclo[7.7.0.0\hat{ }{3,7}.0\hat{ }{11,15}]hexadeca-1(9),2,5,7-tetraen-10-yl]-4-{2-[(3S)-3-(2-methylphenyl)-1,4-oxazepan-4-yl]-7-azaspiro[3.5]nonan-7-yl}-N-{3-nitro-4-[(oxan-4-ylmethyl)amino]benzenesulfonyl}benzamide (20 mg, 25.3%) as a yellow solid. LC-MS (ES, m/z) M+1: 947. ¹HNMR (300 MHz, DMSO-d₆) δ 12.31 (s, 1H), 11.12 (d, J=64.2 Hz, 1H), 8.55 (s, 1H), 8.46-8.24 (m, 1H), 7.62 (d, J=9.2 Hz, 1H), 7.47 (d, J=8.0 Hz, 2H), 7.10 (d, J=13.1 Hz, 4H), 6.94-6.51 (m, 4H), 6.08-5.93 (m, 1H), 4.74-4.18 (m, 3H), 4.16-3.40 (m, 11H), 3.31-2.69 (m, 10H), 2.28 (s, 3H), 1.87 (s, 3H), 1.63 (d, J=12.1 Hz, 4H), 1.47 (s, 3H), 1.29 (d, J=30.0 Hz, 5H).

### Example 67: Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide hydrochloride

Synthesis of N-((3R,4S)-3-hydroxytetrahydro-2H-pyran-4-yl)-4-methylbenzenesulfonamide: A solution of (3R,4S)-4-aminotetrahydro-2H-pyran-3-ol (1.0 g, 8.5 mmol, 1.0 eq) in DCM (20 mL) was treated with TEA (2.1 g, 21.3 mmol, 2.5 eq) at 25° C. under nitrogen atmosphere followed by the addition of TsCl (1.6 g, 9.4 mmol, 1.1 eq) dropwise at 0° C. The resulting mixture was stirred for 4 hours at 25° C. under nitrogen atmosphere. The reaction was quenched by the addition of water (10 mL) at 0° C. The resulting mixture was extracted with CH₂Cl₂ (2×20 mL). The combined organic layers were washed with brine (1×20 mL), dried over anhydrous Na₂SO₄. After filtration, the filtrate was concentrated under reduced pressure. The crude residue was purified by a flash column (silica gel, ethyl acetate/petroleum ether=1:1) to afford N-((3R,4S)-3-hydroxytetrahydro-2H-pyran-4-yl)-4-methylbenzenesulfonamide (1500 mg, 64.7%) as a white solid. LC-MS (ESI, m/z) M+1: 272.

Synthesis of N-[(3R,4S)-3-[(5-bromo-1-{[2-(trimethylsilyl)ethoxy]methyl}pyrrolo[2,3-b]pyridin-6-yl)oxy]oxan-4-yl]-4-methylbenzenesulfonamide: Into a 100 mL round-bottom flask were added N-[(3R,4S)-3-hydroxyoxan-4-yl]-4-methylbenzenesulfonamide (800 mg, 2.9 mmol, 1.0 eq), THE (10 mL). To the above solution was added NaH (354 mg, 8.8 mmol, 3.0 eq, 60%) at 0° C. The mixture was stirred for 30 minutes at 0° C. 5-bromo-6-fluoro-1-{[2-(trimethylsilyl)ethoxy]methyl}pyrrolo[2,3-b]pyridine (1.0 g, 2.9 mmol, 1.0 eq) was added and the mixture was allowed to warm to 50° C. and stirred for overnight under nitrogen atmosphere. The mixture was allowed to cool down to 25° C. The reaction was quenched with sat.NH₄Cl (aq.) and extracted with Ethyl acetate (2×50 mL). The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1:2) to afford N-[(3R,4S)-3-[(5-bromo-1-{[2-(trimethylsilyl)ethoxy]methyl}pyrrolo[2,3-b]pyridin-6-yl)oxy]oxan-4-yl]-4-methylbenzenesulfonamide (400 mg, 22.7%) as a white solid. LC-MS (ES, m/z) M+1: 596/598. ¹HNMR (300 MHz, Chloroform-d) δ 7.99 (s, 1H), 7.39-7.32 (m, 2H), 7.22 (d, J=3.6 Hz, 1H), 7.04 (d, J=8.0 Hz, 2H), 6.44 (d, J=3.6 Hz, 1H), 5.67-5.50 (m, 2H), 4.91 (td, J=9.8, 5.1 Hz, 1H), 4.32-4.07 (m, 1H), 3.95 (dd, J=12.0, 4.5 Hz, 1H), 3.73-3.51 (m, 2H), 3.40 (ddd, J=21.3, 11.7, 10.0 Hz, 3H), 2.37 (s, 3H), 2.05-1.85 (m, 1H), 1.33-1.22 (m, 1H), 1.08-0.93 (m, 2H), −0.00 (s, 9H).

Synthesis of (3R,4S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 250 mL round-bottom flask were added N-[(3R,4S)-3-[(5-bromo-1-{[2-(trimethylsilyl)ethoxy]methyl}pyrrolo[2,3-b]pyridin-6-yl)oxy]oxan-4-yl]-4-methylbenzenesulfonamide (400 mg, 0.7 mmol, 1.0 eq), DMF (5 mL), K₂CO₃ (278 mg, 2.0 mmol, 3.0 eq), CuI (77 mg, 0.4 mmol, 0.6 eq), 1,10-phenanthroline (72 mg, 0.4 mmol, 0.6 eq). The resulting mixture was stirred for overnight at 120° C. under nitrogen atmosphere. The mixture was allowed to cool down to 25° C. The resulting mixture was diluted with water (400 mL). The aqueous layer was extracted with Ethyl acetate (2×200 mL) and the organic layer was washed with brine (1×500 mL), the combined organic layer was dried over anhydrous Na₂SO₄. After filtration, the filtrate was concentrated under reduced pressure. The crude residue was purified by a flash column (silica gel, ethyl acetate/petroleum ether=2:3) to afford (3R,8S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (300 mg, 86.7%) as a colorless oil. LC-MS (ES, m/z) M+1: 516. ¹HNMR (400 MHz, Chloroform-d) δ 8.44 (s, 1H), 7.29-7.23 (m, 3H), 7.13 (d, J=8.2 Hz, 2H), 6.57 (d, J=3.6 Hz, 1H), 5.55 (s, 2H), 4.22 (dd, J=10.9, 4.6 Hz, 1H), 4.06 (dd, J=11.9, 4.4 Hz, 1H), 3.63 (dd, J=10.0, 4.5 Hz, 1H), 3.58-3.46 (m, 4H), 3.31 (t, J=10.3 Hz, 1H), 2.72-2.66 (m, 1H), 2.36 (s, 3H), 2.12 (td, J=12.6, 4.6 Hz, 1H), 1.31-1.23 (m, 2H), 0.91 (dq, J=10.0, 6.6 Hz, 2H), −0.04 (s, 9H).

Synthesis of (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 40 mL vial purged and maintained with an inert atmosphere of nitrogen, was placed Na (93 mg, 4.0 mmol, 7.0 eq), naphthalene (521 mg, 4.0 mmol, 7.0 eq), DME (3 mL). The mixture was stirred for 40 minutes at 25° C. until the formation of Na/naphthalene was complete. Another 250 mL 3-necked round-bottom flask purged and maintained with an inert atmosphere of nitrogen, was placed (3R,8S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (300 mg, 0.6 mmol, 1.0 eq), THE (10 mL) This was followed by the addition of the above solution at −78° C. The resulting solution was stirred for 3 hours at 25° C. The reaction was then quenched by the addition of aqueous NH₄Cl (300 mL). The resulting solution was extracted with Ethyl acetate (3×200 mL) and the organic layers combined. The resulting mixture was washed with brine (1×300 mL). The mixture was dried over anhydrous sodium sulfate. After filtration, the filtrate was concentrated under reduced pressure. The crude residue was purified by a flash column (silica gel, ethyl acetate/petroleum ether=1:1) to afford (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (120 mg, 57.06%) as a yellow oil. LC-MS (ES, m/z) M+1: 362. ¹HNMR (300 MHz, Chloroform-d) δ 7.50 (s, 1H), 7.20 (d, J=3.5 Hz, 1H), 6.36 (d, J=3.6 Hz, 1H), 5.57 (s, 2H), 4.37 (dd, J=10.9, 5.0 Hz, 1H), 4.24-3.98 (m, 2H), 3.66-3.26 (m, 5H), 2.39-2.11 (m, 1H), 2.10-1.84 (m, 1H), 0.96-0.86 (m, 2H), −0.04 (s, 9H).

Synthesis of 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into a 40 mL vial were added (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (54 mg, 0.1 mmol, 1.0 eq) and 2-bromo-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide (185 mg, 0.2 mmol, 1.5 eq), DMSO (3 mL), K₂CO₃ (41 mg, 0.3 mmol, 2.0 eq), CuI (14 mg, 0.07 mmol, 0.5 eq), N,N′-diphenyl-ethanediamide (18 mg, 0.07 mmol, 0.5 eq). The resulting mixture was stirred for 6 hours at 100° C. under nitrogen atmosphere. The mixture was allowed to cool down to 25° C. The resulting mixture was diluted with water (30 mL). The resulting mixture was extracted with Ethyl acetate (3×20 mL). The combined organic layers were washed with brine (1×50 mL), dried over anhydrous Na₂SO₄. After filtration, the filtrate was concentrated under reduced pressure. The crude residue was purified by prep-TLC (dichloromethanel methanol=12:1) to afford 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (80 mg, 48.4%) as a yellow solid. LC-MS (ES, m/z) M+1: 1105.

Synthesis of 2-[(3R,8S)-2,5-dioxa-9,15,17-triazatetracyclo[8.7.0.0\hat{ }{3,8}.0\hat{ }{12,16}]heptadeca. 1(10),11,13,16-tetraen-9-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide hydrochloride: Into a 40 mL vial were added 4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (80 mg, 0.07 mmol, 1.0 eq), TBAF in THE (1.0 M, 3 mL), ethylenediamine (87 mg, 1.4 mmol, 20.0 eq). The resulting mixture was stirred for 8 hours at 70° C. The mixture was allowed to cool down to 25° C. The resulting mixture was diluted with water (100 mL). The resulting mixture was extracted with Ethyl acetate (3×30 mL). The combined organic layers were washed with brine (1×100 mL), dried over anhydrous Na₂SO₄. The crude product was purified by Prep-HPLC with the following conditions (Prep-HPLC-003): Column, SunFire Prep C18 OBD Column, 19*150 mm, 5 μm 10 nm; mobile phase, water (0.05% TFA) and CH₃CN (35% CH₃CN up to 75% in 10 min); Detector, uv. The collected solution was concentrated under vacuum to remove CH₃CN and the resulting solution was dried by lyophilization (added with Conc.HCl (1 drop)). 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)pyrrolidin-1-yl]-7-azaspiro[3.5]nonan-7-yl}-N-{3-nitro-4-[(oxan-4-ylmethyl)amino]benzenesulfonyl}benzamide hydrochloride (13 mg, 17.7%) as a yellow solid. LC-MS (ES, m/z) M+1: 975. ¹HNMR (300 MHz, DMSO-d₆) δ 12.75 (s, 1H), 11.25 (s, 1H), 10.63 (s, 1H), 8.74-6.79 (m, 12H), 6.06 (dd, J=3.4, 1.8 Hz, 1H), 4.79-4.62 (m, 2H), 3.99-3.03 (m, 20H), 2.35-2.07 (m, 5H), 1.75-1.20 (m, 19H).

### Example 68: Preparation of 2-[(3R,8R)-2,5-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of methyl 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoate: Into a 40 mL vial, were placed (3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene (53 mg, 0.14 mmol, 1.0 eq), methyl 2-bromo-4-{2-[(2S)-2-(2-methylphenyl)pyrrolidin-1-yl]-7-azaspiro[3.5]nonan-7-yl}benzoate (126 mg, 0.25 mmol, 1.8 eq), toluene (5 mL), Cs₂CO₃ (184 mg, 0.57 mmol, 4.0 eq), Generation 3rd SPhos precatalyst (44 mg, 0.06 mmol, 0.4 eq). The reaction mixture was stirred for 5 hours at 110° C. under nitrogen atmosphere. The reaction was then quenched by the addition of aqueous water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give methyl 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoate as a light yellow solid (85 mg, 76.0%). LC-MS (ESI, m/z) M+1: 792.

Synthesis of 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoic acid: Into a 40 mL vial, were placed methyl 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoate (85 mg, 0.107 mmol, 1 eq), methanol (3 mL), dioxane (3 mL), NaOH (4 M, 0.6 mL). The reaction mixture was stirred for 4 hours at 70° C. The resulting mixture was concentrated under vacuum. The mixture was acidified to pH=5 with HCl (1 M) and then extracted with EtOAc (2×30 mL). The combined organic layer was washed with brine (100 mL), dried over anhydrous Na₂SO₄. After filtration, the filtrate was concentrated under vacuum to give 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoic acid as a light yellow solid (80 mg, 95.8%). LC-MS (ESI, m/z) M+1: 778.

Synthesis of 4-{2-[(2S)-2-(2-methylphenyl)pyrrolidin-1-yl]-7-azaspiro[3.5]nonan-7-yl}-N-{3-nitro. 4-[(oxan-4-ylmethyl)amino]benzenesulfonyl}-2-[(3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzamide: Into a 40 mL vial, were placed 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoic acid (80 mg, 0.1 mmol, 1.0 eq), 3-nitro-4-[(oxan-4-ylmethyl)amino]benzenesulfonamide (32 mg, 0.1 mmol, 1.0 eq), dichloromethane (3 mL), EDCI (39 mg, 0.2 mmol, 2.0 eq), N,N-dimethylpyridin-4-amine (50 mg, 0.4 mmol, 4.0 eq). The reaction mixture was stirred overnight at 30° C. The resulting mixture was concentrated under vacuum. The residue was purified by Prep-TLC (dichloromethane/methanol=12:1) to give 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzamide (assumed) as a yellow solid (44 mg, 39.8%). LC-MS (ESI, m/z) M+1: 1075.

Synthesis of 2-[(3R,8R)-2,5-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide: Into a 40 mL vial, were placed 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzamide (44 mg, 0.04 mmol, 1.0 eq), TBAF-THF (5 mL, 1 M), ethylenediamine (49 mg, 0.8 mmol, 20.0 eq). The reaction mixture was stirred for 7 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and acetonitrile (45% acetonitrile up to 85% in 10 min); Detector, UV. Finally, 2-[(3R,8R)-2,5-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide (assumed) was obtained as a yellow solid (16 mg, 41.4%). LC-MS (ESI, m/z) M+1: 945. ¹HNMR (400 MHz, DMSO-d₆) δ 11.95 (s, 1H), 11.34 (s, 1H), 8.82-8.26 (m, 2H), 7.70 (s, 1H), 7.51 (d, J=7.8 Hz, 2H), 7.27 (t, J=2.9 Hz, 1H), 7.20-6.55 (m, 6H), 6.19 (dd, J=3.4, 1.9 Hz, 1H), 4.16 (s, 1H), 3.97-3.70 (m, 3H), 3.66-3.39 (m, 3H), 3.36 (s, 4H), 3.29-3.19 (m, 5H), 3.17-2.93 (m, 5H), 2.43-2.02 (m, 6H), 1.78 (d, J=16.2 Hz, 5H), 1.63-1.37 (m, 9H), 1.24 (dq, J=11.2, 6.1 Hz, 3H).

### Example 69: Preparation of 2-[(3S,8S)-2,5-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of methyl 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoate: Into a 40 mL vial, were placed (3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene (72 mg, 0.19 mmol, 1.0 eq), methyl 2-bromo-4-{2-[(2S)-2-(2-methylphenyl)pyrrolidin-1-yl]-7-azaspiro[3.5]nonan-7-yl}benzoate (171 mg, 0.34 mmol, 1.8 eq), toluene (5 mL), Cs₂CO₃ (250 mg, 0.77 mmol, 4.0 eq), Generation 3rd SPhos precatalyst (60 mg, 0.08 mmol, 0.4 eq). The reaction mixture was stirred for 5 hours at 110° C. under nitrogen atmosphere. The reaction was then quenched by the addition of aqueous water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give methyl 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoate (assumed) as a yellow oil (75 mg, 49.4%). LC-MS (ESI, m/z) M+1: 792.

Synthesis of 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoic acid: Into a 40 mL vial, were placed methyl 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoate (assumed) (75 mg, 0.09 mmol, 0.01 eq), methanol (3 mL), dioxane (3 mL), NaOH (4 M, 0.6 mL). The reaction mixture was stirred for 4 hours at 70° C. The resulting mixture was concentrated under vacuum. The mixture was acidified to pH 5 with HCl (1 M) and then extracted with EtOAc (2×30 mL). The combined organic layer was washed with brine (100 mL), dried over anhydrous Na₂SO₄. After filtration, the filtrate was concentrated under vacuum to give 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoic acid (assumed) as a light yellow solid (70 mg, 95.0%). LC-MS (ESI, m/z) M+1: 778.

Synthesis of 4-{2-[(2S)-2-(2-methylphenyl)pyrrolidin-1-yl]-7-azaspiro[3.5]nonan-7-yl}-N-{3-nitro. 4-[(oxan-4-ylmethyl)amino]benzenesulfonyl}-2-[(3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzamide: Into a 40 mL vial, were placed 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzoic acid (assumed) (70 mg, 0.09 mmol, 1.0 eq), 3-nitro-4-[(oxan-4-ylmethyl)amino]benzenesulfonamide (28 mg, 0.09 mmol, 1.0 eq), dichloromethane (3 mL), EDCI (34 mg, 0.18 mmol, 2.0 eq), N,N-dimethylpyridin-4-amine (44 mg, 0.36 mmol, 4.0 eq). The reaction mixture was stirred overnight at 30° C. The resulting mixture was concentrated under vacuum. The residue was purified by Prep-TLC (dichloromethane/methanol=12:1) to give 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzamide (assumed) as a yellow solid (55 mg, 56.8%). LC-MS (ESI, m/z) M+1: 1075.

Synthesis of 2-[(3S,8S)-2,5-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide: Into a 40 mL vial, were placed 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraen-10-yl]benzamide (55 mg, 0.05 mmol, 1.0 eq), TBAF-THF (5 mL, 1 M), ethylenediamine (62 mg, 1.0 mmol, 20.0 eq). The reaction mixture was stirred for 7 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and acetonitrile (45% acetonitrile up to 85% in 10 min); Detector, UV. Finally, 2-[(3S,8S)-2,5-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide was obtained as a yellow solid (24 mg, 49.6%). LC-MS (ESI, m/z) M+1: 945. ¹HNMR (400 MHz, DMSO-d₆) δ 11.95 (s, 1H), 11.34 (s, 1H), 8.82-8.26 (m, 2H), 7.70 (s, 1H), 7.51 (d, J=7.8 Hz, 2H), 7.27 (t, J=2.9 Hz, 1H), 7.20-6.55 (m, 6H), 6.19 (dd, J=3.4, 1.9 Hz, 1H), 4.16 (s, 1H), 3.97-3.70 (m, 3H), 3.66-3.39 (m, 5H), 3.36 (s, 4H), 3.29-3.19 (m, 5H), 3.17-2.93 (m, 5H), 2.43-2.02 (m, 6H), 1.78 (d, J=16.2 Hz, 5H), 1.63-1.37 (m, 8H), 1.24 (dq, J=11.2, 6.1 Hz, 3H).

### Example 70: Preparation of 2-[(3S,8S)-2,6-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of methyl 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoate: Into a 40 mL vial, were placed (3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene (75 mg, 0.2 mmol, 1.0 eq), methyl 2-bromo-4-{2-[(2S)-2-(2-methylphenyl)pyrrolidin-1-yl]-7-azaspiro[3.5]nonan-7-yl}benzoate (179 mg, 0.4 mmol, 2.0 eq), toluene (5 mL), Cs₂CO₃ (260 mg, 0.8 mmol, 4.0 eq), Generation 3rd SPhos precatalyst (62 mg, 0.1 mmol, 0.4 eq). The reaction mixture was stirred for 5 hours at 110° C. under nitrogen atmosphere. The reaction was then quenched by the addition of aqueous water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give methyl 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoate (assumed) as a yellow solid (75 mg, 47.4%). LC-MS (ESI, m/z) M+1: 792.

Synthesis of 4-{2-[(2S)-2-(2-methylphenyl)pyrrolidin-1-yl]-7-azaspiro[3.5]nonan-7-yl}-2-[(3S,8S)-16. {[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoic acid: Into a 40 mL vial, were placed methyl 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoate (75 mg, 0.1 mmol, 1.0 eq), methanol (3 mL), dioxane (3 mL), NaOH (4 M, 0.6 mL). The reaction mixture was stirred for 4 hours at 70° C. The resulting mixture was concentrated under vacuum. The mixture was acidified to pH=5 with HCl (1 M) and then extracted with EtOAc (2×30 mL). The combined organic layer was washed with brine (100 mL), dried over anhydrous Na₂SO₄. After filtration, the filtrate was concentrated under vacuum to give 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoic acid as a light yellow solid (70 mg, 95.0%). LC-MS (ESI, m/z) M+1: 778.

Synthesis of 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzamide: Into a 40 mL vial, were placed 4-{2-[(2S)-2-(2-methylphenyl)pyrrolidin-1-yl]-7-azaspiro[3.5]nonan-7-yl}-2-[(3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoic acid (assumed) (70 mg, 0.1 mmol, 1.0 eq), 3-nitro-4-[(oxan-4-ylmethyl)amino]benzenesulfonamide (28 mg, 0.1 mmol, 1.0 eq), dichloromethane (3 mL), EDCI (34 mg, 0.2 mmol, 2.0 eq), N,N-dimethylpyridin-4-amine (44 mg, 0.4 mmol, 4.0 eq). The reaction mixture was stirred overnight at 30° C. The resulting mixture was concentrated under vacuum. The residue was purified by Prep-TLC (dichloromethane/methanol=12:1) to give 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzamide as a yellow solid (60 mg, 62.0%). LC-MS (ESI, m/z) M+1: 1076.

Synthesis of 2-[(3S,8S)-2,6-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide: Into a 40 mL vial, were placed 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzamide (60 mg, 0.06 mmol, 1.0 eq), TBAF-THF (5 mL, 1 M), ethylenediamine (67 mg, 1.1 mmol, 20.0 eq). The reaction mixture was stirred for 7 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and acetonitrile (45% acetonitrile up to 85% in 10 min); Detector, UV. Finally, 2-[(3S,8S)-2,6-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide was obtained as a yellow solid (17 mg, 32.2%). LC-MS (ESI, m/z) M+1: 945. ¹HNMR (300 MHz, DMSO-d₆) δ 11.99 (s, 1H), 11.31 (s, 1H), 8.49 (s, 2H), 7.67 (d, J=9.1 Hz, 1H), 7.52 (d, J=7.7 Hz, 2H), 7.26 (t, J=3.0 Hz, 1H), 7.12 (d, J=17.6 Hz, 3H), 6.88 (d, J=9.4 Hz, 2H), 6.75 (s, 1H), 6.17 (dd, J=3.4, 1.9 Hz, 1H), 3.99 (s, 1H), 3.82 (t, J=13.2 Hz, 4H), 3.58 (s, 4H), 3.30-2.79 (m, 11H), 2.30 (s, 4H), 2.25-2.05 (m, 3H), 2.00-1.11 (m, 18H).

### Example 71: Preparation of 2-[(3R,8R)-2,6-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide

Synthesis of methyl 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoate: Into a 40 mL vial, were placed (3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene (80 mg, 0.2 mmol, 1.0 eq), methyl 2-bromo-4-{2-[(2S)-2-(2-methylphenyl)pyrrolidin-1-yl]-7-azaspiro[3.5]nonan-7-yl}benzoate (190 mg, 0.4 mmol, 1.8 eq), toluene (5 mL), Cs₂CO₃ (278 mg, 0.8 mmol, 4.0 eq), Generation 3rd SPhos precatalyst (67 mg, 0.09 mmol, 0.4 eq). The reaction mixture was stirred for 5 hours at 110° C. under nitrogen atmosphere. The reaction was then quenched by the addition of aqueous water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give methyl 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoate as a light yellow oil (80 mg, 47.4%). LC-MS (ESI, m/z) M+1: 792.

Synthesis of 4-{2-[(2S)-2-(2-methylphenyl)pyrrolidin-1-yl]-7-azaspiro[3.5]nonan-7-yl}-2-[(3R,8R)-16. {[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoic acid: Into a 40 mL vial, were placed methyl 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoate (80 mg, 0.1 mmol, 1.0 eq), methanol (3 mL), dioxane (3 mL), NaOH (4 M, 0.6 mL). The reaction mixture was stirred for 8 hours at 70° C. The resulting mixture was concentrated under vacuum. The mixture was acidified to pH=5 with HCl (1 M) and then extracted with EtOAc (2×30 mL). The combined organic layer was washed with brine (100 mL), dried over anhydrous Na₂SO₄. After filtration, the filtrate was concentrated under vacuum to give 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoic acid (assumed) as a light yellow solid (75 mg, 95.4%). LC-MS (ESI, m/z) M+1: 778.

Synthesis of 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzamide: Into a 40 mL vial, were placed 4-{2-[(2S)-2-(2-methylphenyl)pyrrolidin-1-yl]-7-azaspiro[3.5]nonan-7-yl}-2-[(3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzoic acid (75 mg, 0.1 mmol, 1.0 eq), 3-nitro-4-[(oxan-4-ylmethyl)amino]benzenesulfonamide (30 mg, 0.1 mmol, 1.0 eq), dichloromethane (3 mL), EDCI (37 mg, 0.2 mmol, 2.0 eq), N,N-dimethylpyridin-4-amine (47 mg, 0.4 mmol, 4.0 eq). The reaction mixture was stirred overnight at 30° C. The resulting mixture was concentrated under vacuum. The residue was purified by Prep-TLC (dichloromethane/methanol=12:1) to give 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzamide as a yellow solid (60 mg, 57.9%). LC-MS (ESI, m/z) M+1: 1076.

Synthesis of 2-[(3R,8R)-2,6-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide: Into a 40 mL vial, were placed 4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}-2-[(3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraen-10-yl]benzamide (60 mg, 0.06 mmol, 1.0 eq), TBAF-THF (5 mL, 1 M), ethylenediamine (67 mg, 1.1 mmol, 20.0 eq). The reaction mixture was stirred for 7 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and acetonitrile (45% acetonitrile up to 85% in 10 min); Detector, UV. Finally, 2-[(3R,8R)-2,6-dioxa-10,16,18-triazatetracyclo[9.7.0.0\hat{ }{3,8}.0\hat{ }{13,17}]octadeca-1(11),12,14,17-tetraen-10-yl]-4-{2-[(2S)-2-(2-methylphenyl)pyrrolidin-1-yl]-7-azaspiro[3.5]nonan-7-yl}-N-{3-nitro-4-[(oxan-4-ylmethyl)amino]benzenesulfonyl}benzamide was obtained as a yellow solid (33 mg, 62.6%). LC-MS (ESI, m/z) M+1: 945. ¹HNMR (300 MHz, DMSO-d₆) δ 11.99 (s, 1H), 11.31 (s, 1H), 8.49 (s, 2H), 7.67 (d, J=9.1 Hz, 1H), 7.52 (d, J=7.7 Hz, 2H), 7.26 (t, J=3.0 Hz, 1H), 7.12 (d, J=17.6 Hz, 3H), 6.88 (d, J=9.4 Hz, 2H), 6.75 (s, 1H), 6.17 (dd, J=3.4, 1.9 Hz, 1H), 3.99 (s, 1H), 3.82 (t, J=13.2 Hz, 4H), 3.58 (s, 4H), 3.30-2.79 (m, 11H), 2.30 (s, 4H), 2.25-2.05 (m, 3H), 2.00-1.11 (m, 18H).

### Example 72: Preparation of N-((3-nitro-4-(((tetrahydro-2H-pyran-4-yl)methyl)amino)phenyl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide

Synthesis of methyl 4-(2-((S)-2-(o-tolyl)pyrrolidin-1-yl)-7-azaspiro[3.5]nonan-7-yl)-2-((6aR,9aS)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoate: Into a 40 mL vial, were placed (6aR,9aS)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (75 mg, 0.2 mmol, 1.0 eq), methyl 2-bromo-4-{2-[(2S)-2-(2-methylphenyl)pyrrolidin-1-yl]-7-azaspiro[3.5]nonan-7-yl}benzoate (179 mg, 0.4 mmol, 2.0 eq), toluene (5 mL), Cs₂CO₃ (260 mg, 0.8 mmol, 4.0 eq), Generation 3rd SPhos precatalyst (62 mg, 0.1 mmol, 0.4 eq). The reaction mixture was stirred for 5 hours at 110° C. under nitrogen atmosphere. The reaction was then quenched by the addition of aqueous water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give methyl 4-(2-((S)-2-(o-tolyl)pyrrolidin-1-yl)-7-azaspiro[3.5]nonan-7-yl)-2-((6aR,9aS)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoate as a yellow solid (75 mg, 47.4%). LC-MS (ESI, m/z) M+1: 778.

Synthesis of 4-(2-((S)-2-(o-tolyl)pyrrolidin-1-yl)-7-azaspiro[3.5]nonan-7-yl)-2-((6aR,9aS)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoic acid: Into a 40 mL vial, were placed methyl 4-(2-((S)-2-(o-tolyl)pyrrolidin-1-yl)-7-azaspiro[3.5]nonan-7-yl)-2-((6aR,9aS)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoate (75 mg, 0.1 mmol, 1.0 eq), methanol (3 mL), dioxane (3 mL), NaOH (4 M, 0.6 mL). The reaction mixture was stirred for 4 hours at 70° C. The resulting mixture was concentrated under vacuum. The mixture was acidified to pH=5 with HCl (1 M) and then extracted with EtOAc (2×30 mL). The combined organic layer was washed with brine (100 mL), dried over anhydrous Na₂SO₄. After filtration, the filtrate was concentrated under vacuum to give 4-(2-((S)-2-(o-tolyl)pyrrolidin-1-yl)-7-azaspiro[3.5]nonan-7-yl)-2-((6aR,9aS)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoic acid as a light yellow solid (70 mg, 95.0%). LC. MS (ESI, m/z) M+1: 764.

Synthesis of N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)-2-((6aR,9aS)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzamide: Into a 40 mL vial, were placed 4-(2-((S)-2-(o-tolyl)pyrrolidin-1-yl)-7-azaspiro[3.5]nonan-7-yl)-2-((6aR,9aS)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoic acid (assumed) (70 mg, 0.1 mmol, 1.0 eq), 3-nitro-4-[(oxan-4-ylmethyl)amino]benzenesulfonamide (28 mg, 0.1 mmol, 1.0 eq), dichloromethane (3 mL), EDCI (34 mg, 0.2 mmol, 2.0 eq), N,N-dimethylpyridin-4-amine (44 mg, 0.4 mmol, 4.0 eq). The reaction mixture was stirred overnight at 30° C. The resulting mixture was concentrated under vacuum. The residue was purified by Prep-TLC (dichloromethane/methanol=12:1) to give N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)-2-((6aR,9aS)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzamide as a yellow solid (60 mg, 62.0%). LC-MS (ESI, m/z) M+1: 1062.

Synthesis of N-((3-nitro-4-(((tetrahydro-2H-pyran-4-yl)methyl)amino)phenyl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide: Into a 40 mL vial, were placed N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)-2-((6aR,9aS)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzamide (60 mg, 0.06 mmol, 1.0 eq), TBAF-THF (5 mL, 1 M), ethylenediamine (67 mg, 1.1 mmol, 20.0 eq). The reaction mixture was stirred for 7 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and acetonitrile (45% acetonitrile up to 85% in 10 min); Detector, UV. Finally, N-((3-nitro-4-(((tetrahydro-2H-pyran-4-yl)methyl)amino)phenyl)sulfonyl)-2-((6aR,9aS)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide was obtained as a yellow solid (17 mg, 32.2%). LC-MS (ESI, m/z) M+1: 931. ¹HNMR (400 MHz, Chloroform-d) δ 11.76 (s, 1H), 8.84 (s, 1H), 8.64 (s, 1H), 8.36 (s, 1H), 8.04 (s, 1H), 7.92 (d, J=8.8 Hz, 2H), 7.62 (s, 1H), 7.44 (s, 1H), 6.74 (s, 4H), 6.24 (s, 1H), 5.37 (t, J=4.8 Hz, 2H), 4.44 (t, J=7.6 Hz, 2H), 4.28 (s, 1H), 4.16 (s, 2H), 4.04 (d, J=12.0 Hz, 5H), 3.92 (s, 1H), 3.64 (t, J=9.6 Hz, 3H), 3.56 (s, 1H), 3.44 (t, J=11.6 Hz, 5H), 3.32 (t, J=11.6 Hz, 4H), 3.16 (s, 5H), 2.84 (s, 1H), 2.62 (s, 1H), 2.04 (d, J=6.8 Hz, 2H), 1.92 (s, 3H), 1.84 (s, 3H), 0.92 (t, J=6.6 Hz, 2H).

### Example 73: Preparation of N-((3-nitro-4-(((tetrahydro-2H-pyran-4-yl)methyl)amino)phenyl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide

Synthesis of methyl 4-(2-((S)-2-(o-tolyl)pyrrolidin-1-yl)-7-azaspiro[3.5]nonan-7-yl)-2-((6aS,9aR)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoate: Into a 40 mL vial, were placed (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (80 mg, 0.2 mmol, 1.0 eq), methyl 2-bromo-4-{2-[(2S)-2-(2-methylphenyl)pyrrolidin-1-yl]-7-azaspiro[3.5]nonan-7-yl}benzoate (190 mg, 0.4 mmol, 1.8 eq), toluene (5 mL), Cs₂CO₃ (278 mg, 0.8 mmol, 4.0 eq), Generation 3rd SPhos precatalyst (67 mg, 0.09 mmol, 0.4 eq). The reaction mixture was stirred for 5 hours at 110° C. under nitrogen atmosphere. The reaction was then quenched by the addition of aqueous water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give methyl 4-(2-((S)-2-(o-tolyl)pyrrolidin-1-yl)-7-azaspiro[3.5]nonan-7-yl)-2-((6aS,9aR)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoate (80 mg, 47.4%). LC-MS (ESI, m/z) M+1: 778.

Synthesis of 4-(2-((S)-2-(o-tolyl)pyrrolidin-1-yl)-7-azaspiro[3.5]nonan-7-yl)-2-((6aS,9aR)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoic acid: Into a 40 mL vial, were placed methyl 4-(2-((S)-2-(o-tolyl)pyrrolidin-1-yl)-7-azaspiro[3.5]nonan-7-yl)-2-((6aS,9aR)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoate (80 mg, 0.1 mmol, 1.0 eq), methanol (3 mL), dioxane (3 mL), NaOH (4 M, 0.6 mL). The reaction mixture was stirred for 8 hours at 70° C. The resulting mixture was concentrated under vacuum. The mixture was acidified to pH=5 with HCl (1 M) and then extracted with EtOAc (2×30 mL). The combined organic layer was washed with brine (100 mL), dried over anhydrous Na₂SO₄. After filtration, the filtrate was concentrated under vacuum to give 4-(2-((S)-2-(o-tolyl)pyrrolidin-1-yl)-7-azaspiro[3.5]nonan-7-yl)-2-((6aS,9aR)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoic acid as a light yellow solid (75 mg, 95.4%). LC. MS (ESI, m/z) M+1: 764.

Synthesis of N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)-2-((6aS,9aR)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzamide: Into a 40 mL vial, were placed 4-(2-((S)-2-(o-tolyl)pyrrolidin-1-yl)-7-azaspiro[3.5]nonan-7-yl)-2-((6aS,9aR)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzoic acid (75 mg, 0.1 mmol, 1.0 eq), 3-nitro-4-[(oxan-4-ylmethyl)amino]benzenesulfonamide (30 mg, 0.1 mmol, 1.0 eq), dichloromethane (3 mL), EDCI (37 mg, 0.2 mmol, 2.0 eq), N,N-dimethylpyridin-4-amine (47 mg, 0.4 mmol, 4.0 eq). The reaction mixture was stirred overnight at 30° C. The resulting mixture was concentrated under vacuum. The residue was purified by Prep-TLC (dichloromethane/methanol=12:1) to give N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)-2-((6aS,9aR)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzamide as a yellow solid (60 mg, 57.9%). LC-MS (ESI, m/z) M+1: 1062.

Synthesis of N-((3-nitro-4-(((tetrahydro-2H-pyran-4-yl)methyl)amino)phenyl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide: Into a 40 mL vial, were placed N-((3-nitro-4-(((tetrahydro-2H-pyran-4-yl)methyl)amino)phenyl)sulfonyl)-4-(2-((S)-2-(o-tolyl)pyrrolidin-1-yl)-7-azaspiro[3.5]nonan-7-yl)-2-((6aS,9aR)-1-((2-(trimethylsilyl)ethoxy)methyl)-6a,7,9,9a-tetrahydro-1H-furo[3,4-f]pyrrolo[3′,2′:5,6]pyrido[2,3-b][1,4]oxazepin-5(6H)-yl)benzamide (60 mg, 0.06 mmol, 1.0 eq), TBAF-THF (5 mL, 1 M), ethylenediamine (67 mg, 1.1 mmol, 20.0 eq). The reaction mixture was stirred for 7 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and acetonitrile (45% acetonitrile up to 85% in 10 min); Detector, UV. Finally, N-((3-nitro-4-(((tetrahydro-2H-pyran-4-yl)methyl)amino)phenyl)sulfonyl)-2-((6aS,9aR)-6a,7,9,9a-tetrahydro-1H-furo[3,4-f]pyrrolo[3′,2′:5,6]pyrido[2,3-b][1,4]oxazepin-5(6H)-yl)-4-(2-((S)-2-(o-tolyl)pyrrolidin-1-yl)-7-azaspiro[3.5]nonan-7-yl)benzamide was obtained as a yellow solid (33 mg, 62.6%). LC-MS (ESI, m/z) M+1: 931. ¹HNMR (400 MHz, Chloroform-d) δ 11.76 (s, 1H), 8.84 (s, 1H), 8.68 (s, 2H), 8.44 (s, 1H), 8.36 (s, 2H), 8.04 (s, 1H), 7.92 (d, J=8.8 Hz, 3H), 7.62 (s, 1H), 7.20 (s, 2H), 6.24 (s, 2H), 5.36 (t, J=4.8 Hz, 2H), 4.44 (t, J=7.6 Hz, 3H), 4.28 (s, 1H), 4.16 (s, 2H), 4.04 (d, J=11.6 Hz, 6H), 3.92 (s, 1H), 3.68 (t, J=9.6 Hz, 3H), 3.44 (t, J=11.6 Hz, 6H), 3.16 (s, 6H), 2.84 (s, 1H), 2.62 (s, 1H), 2.24 (t, J=7.6 Hz, 3H), 2.12 (s, 1H), 2.07-2.01 (m, 3H), 1.92 (s, 3H).

### Example 74: Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(3R)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride

Synthesis of N-((4-((((1r,4r)-4-hydroxy-4-methylcyclohexyl)methyl)amino)-3-nitrophenyl)sulfonyl)-4-(2-((R)-3-(2-isopropylphenyl)morpholino)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial, were placed (3R)-3-(2-isopropylphenyl)morpholine (19 mg, 0.09 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (87 mg, 0.09 mmol, 1.0 eq), methanol (2 mL), ZnCl₂ (25 mg, 0.18 mmol, 2.0 eq), NaBH₃CN (29 mg, 0.46 mmol, 5.0 eq). The reaction mixture was stirred overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1) to give 4-{2-[(3R)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as a yellow solid (80 mg, 76.3%). LC-MS (ESI, m/z) M+1: 1133.

Synthesis of 2-[(3R,8S)-2,5-dioxa-9,15,17-triazatetracyclo[8.7.0.0\hat{ }{3,8}.0\hat{ }{12,16}]heptadeca. 1(10),11,13,16-tetraen-9-yl]-4-{2-[(3R)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride: Into a 40 mL vial, were placed 4-{2-[(3R)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (80 mg, 0.07 mmol, 1.0 eq), TBAF-THF (5 mL, 1 M), ethylenediamine (85 mg, 1.4 mmol, 20.0 eq). The reaction mixture was stirred for 5 hours at 70° C. The resulting mixture was quenched by the addition of water (100 mL), and extracted with EtOAc (3×30 mL). The combined organic layer was washed with brine (100 mL), dried over anhydrous Na₂SO₄.

After filtration, the filtrate was concentrated under vacuum. The residue was purified by Prep-TLC (dichloromethane/methanol=11:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc.HCl (1 drop)). Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(3R)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride was obtained as a yellow solid (15 mg, 20.4%). LC-MS (ESI, m/z) M+1: 1003. ¹HNMR (400 MHz, DMSO-d₆) δ 12.75 (s, 1H), 11.96 (d, J=9.0 Hz, 1H), 11.25 (s, 1H), 8.57 (t, J=5.8 Hz, 1H), 8.39 (dd, J=8.6, 2.3 Hz, 1H), 8.05 (d, J=7.9 Hz, 1H), 7.78 (d, J=9.1 Hz, 1H), 7.41 (dt, J=8.6, 6.7 Hz, 3H), 7.25 (td, J=7.3, 6.6, 2.1 Hz, 1H), 7.16-7.10 (m, 1H), 6.98 (dd, J=9.3, 2.4 Hz, 1H), 6.88-6.80 (m, 2H), 6.44 (s, 1H), 6.05 (dd, J=3.4, 1.9 Hz, 1H), 4.71 (s, 1H), 4.26-4.03 (m, 4H), 3.92 (t, J=11.7 Hz, 1H), 3.85-3.74 (m, 4H), 3.51-2.98 (m, 11H), 2.23 (t, J=10.1 Hz, 1H), 2.15-2.00 (m, 1H), 1.68 (d, J=13.4 Hz, 2H), 1.62-1.21 (m, 15H), 1.17-1.08 (m, 8H), 0.79 (s, 1H).

### Example 75: Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(3S)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride

Synthesis of 4-{2-[(3S)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca. 1(10),11,13,16-tetraen-9-yl]benzamide: Into an 8 mL vial, were placed (3S)-3-(2-isopropylphenyl)morpholine (19 mg, 0.09 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (87 mg, 0.09 mmol, 1.0 eq), methanol (2 mL), ZnCl₂ (25 mg, 0.18 mmol, 2.0 eq), NaBH₃CN (29 mg, 0.46 mmol, 5.0 eq). The reaction mixture was stirred overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1) to give 4-{2-[(3S)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as a yellow solid (80 mg, 76.3%). LC-MS (ESI, m/z) M+1: 1133.

Synthesis of 2-[(3R,8S)-2,5-dioxa-9,15,17-triazatetracyclo[8.7.0.0\hat{ }{3,8}.0\hat{ }{12,16}]heptadeca. 1(10),11,13,16-tetraen-9-yl]-4-{2-[(3S)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride: Into a 40 mL vial, were placed 4-{2-[(3S)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (80 mg, 0.07 mmol, 1.0 eq), TBAF-THF (5 mL, 1 M), ethylenediamine (85 mg, 1.4 mmol, 20.0 eq). The reaction mixture was stirred for 5 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=11:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc.HCl (1 drop)). Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(3S)-3-(2-isopropylphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride was obtained as a yellow solid (26 mg, 35.4%). LC-MS (ESI, m/z) M-HCl+1: 1003. ¹HNMR (400 MHz, DMSO-d₆) δ 12.75 (s, 1H), 11.96 (d, J=9.0 Hz, 1H), 11.25 (s, 1H), 8.57 (t, J=5.8 Hz, 1H), 8.39 (dd, J=8.6, 2.3 Hz, 1H), 8.05 (d, J=7.9 Hz, 1H), 7.78 (d, J=9.1 Hz, 1H), 7.41 (dt, J=8.6, 6.7 Hz, 3H), 7.25 (td, J=7.3, 6.6, 2.1 Hz, 1H), 7.16-7.10 (m, 1H), 6.98 (dd, J=9.3, 2.4 Hz, 1H), 6.88-6.80 (m, 2H), 6.44 (s, 1H), 6.05 (dd, J=3.4, 1.9 Hz, 1H), 4.71 (s, 1H), 4.26-4.03 (m, 4H), 3.92 (t, J=11.7 Hz, 1H), 3.85-3.74 (m, 4H), 3.51-2.98 (m, 11H), 2.23 (t, J=10.1 Hz, 1H), 2.15-2.00 (m, 1H), 1.68 (d, J=13.4 Hz, 2H), 1.62-1.21 (m, 15H), 1.17-1.08 (m, 8H), 0.79 (s, 1H).

### Example 76: Preparation of N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide hydrochloride

Synthesis of (R)-2-chloro-N-(2-hydroxy-1-(o-tolyl)ethyl)acetamide: Into a 250 mL roundbottom flask purged and maintained with an inert atmosphere of nitrogen. To a stirred solution of (R)-2-amino-2-(o-tolyl)ethan-1-ol (2.2 g, 14.6 mmol, 1.0 eq) and triethylamine (2.4 g, 23.3 mmol, 1.5 eq) in CH₂Cl₂ (22 mL) was added 2-chloroacetyl chloride (2.0 g, 17.5 mmol, 1.2 eq) in portions at 0° C. The resulting mixture was stirred for 3 hours at 0° C. The resulting mixture was concentrated under vacuum. The crude residue was applied onto a silica gel column and eluted with ethyl acetate/petroleum ether=1:3 to give (R)-2-chloro-N-(2-hydroxy-1-(o-tolyl)ethyl)acetamide as a white oil (1.5 g, 45.9%). LC-MS (ESI, m/z) M+1: 228.

Synthesis of (R)-5-(o-tolyl)morpholin-3-one: Into a 250 mL 3-necked round-bottom flask were added (R)-2-chloro-N-(2-hydroxy-1-(o-tolyl)ethyl)acetamide (1.5 g, 6.4 mmol, 1.0 eq), THE (30 mL). After that, to the above mixture was added NaH (0.6 g, 12.8 mmol, 2.0 eq, 60%) in portions at 0° C. The resulting mixture was stirred for additional 0.5 hour at 0° C. The resulting mixture was stirred for additional overnight at 25° C. The resulting mixture was quenched by the addition of water (10 mL) and extracted with CH₂Cl₂ (2×20 mL). The combined organic layer was washed with brine (20 mL), dried over anhydrous Na₂SO₄. After filtration, the resulting mixture was concentrated under vacuum. The crude residue was applied onto a silica gel column and eluted with ethyl acetate/petroleum ether=1:1 to give (R)-5-(o-tolyl)morpholin-3-one as a white solid (0.7 g, 67.9%). LC-MS (ESI, m/z) M+1: 192.

Synthesis of (R)-3-(o-tolyl)morpholine: Into a 40 ml glass bottle flask purged and maintained with an inert atmosphere of nitrogen. To a stirred solution (R)-5-(o-tolyl)morpholin-3-one (600 mg, 3.1 mmol, 1.0 eq) in THE (6 mL) was added LiAlH₄ (490 mg, 3.1 mmol, 4.0 eq) in portions at 0° C. under N₂ atmosphere. The resulting mixture was stirred for 16 hours at 25° C. under N₂ atmosphere. The reaction was quenched with water (0.49 mL), NaOH (15% aq, 0.49 mL) and water (1.5 mL) at 0° C. and the filter cake was washed with MeOH (3×10 mL). The resulting mixture was concentrated under vacuum. The crude residue was applied onto a silica gel column and eluted with ethyl acetate/petroleum ether=2:1 to give (R)-3-(o-tolyl)morpholine as a white oil (400 mg, 77.3%). LC-MS (ESI, m/z) M+1: 178.

Synthesis of N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial, were placed (R)-3-(o-tolyl)morpholine (19 mg, 0.09 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (87 mg, 0.09 mmol, 1.0 eq), methanol (2 mL), ZnCl₂ (25 mg, 0.18 mmol, 2.0 eq), NaBH₃CN (29 mg, 0.46 mmol, 5.0 eq). The reaction mixture was stirred overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1) to give N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (80 mg, 76.3%). LC-MS (ESI, m/z) M+1: 1105.

Synthesis of N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide hydrochloride: Into a 40 mL vial, were placed N-((4-((((1r,4r)-4-hydroxy-4-methylcyclohexyl)methyl)amino)-3-nitrophenyl)sulfonyl)-4-(2-((R)-3-(o-tolyl)morpholino)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (80 mg, 0.07 mmol, 1.0 eq), TBAF-THF (5 mL, 1 M), ethylenediamine (85 mg, 1.4 mmol, 20.0 eq). The reaction mixture was stirred for 5 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=11:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc.HCl (1 drop)). Finally, N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)morpholino)-7-azaspiro[3.5]nonan-7-yl)benzamide hydrochloride was obtained as a yellow solid (15 mg, 20.4%). LC-MS (ESI, m/z) M-HCl+1: 975. ¹HNMR (300 MHz, DMSO-d₆) δ 12.75 (s, 1H), 11.25 (s, 1H), 11.07 (s, 1H), 8.55 (d, J=5.5 Hz, 1H), 8.37 (d, J=2.4 Hz, 1H), 7.86-7.74 (m, 2H), 7.47 (d, J=6.0 Hz, 2H), 7.11 (d, J=7.5 Hz, 4H), 6.84 (d, J=9.9 Hz, 2H), 6.44 (s, 1H), 6.05 (d, J=1.8 Hz, 1H), 4.11 (s, 2H), 3.47-3.31 (m, 4H), 3.27 (s, 2H), 3.21-3.08 (m, 4H), 2.43 (s, 3H), 2.28 (s, 4H), 2.10 (d, J=8.7 Hz, 2H), 1.68 (d, J=13.2 Hz, 3H), 1.56 (d, J=13.2 Hz, 5H), 1.38 (s, 3H), 1.31 (t, J=7.2 Hz, 7H), 1.15-0.95 (m, 6H).

### Example 77: Preparation of N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide hydrochloride

Synthesis of tert-butyl (R)-(2-hydroxy-1-(o-tolyl)ethyl)carbamate: Into a 250 mL round-bottom flask were added (R)-2-amino-2-(o-tolyl)ethan-1-ol (4.3 g, 28.1 mmol, 1.0 eq), CH₂Cl₂ (50 mL), TEA (5.7 g, 56.3 mmol, 2.0 eq), DMAP (344 mg, 2.8 mmol, 0.1 eq), Boc₂O (7.4 g, 33.9 mmol, 1.2 eq). The resulting mixture was stirred overnight at 25° C. The resulting mixture was diluted with HCl (0.2 M). The resulting mixture was extracted with CH₂Cl₂ (3×50 mL). The combined organic layers were washed with brine (300 mL), dried over anhydrous Na₂SO₄. After filtration, the resulting mixture was concentrated under vacuum. The crude product was re-crystallized from ethyl acetate/petroleum ether (1:2) to give tert-butyl (R)-(2-hydroxy-1-(o-tolyl)ethyl)carbamate as a white solid (5 g, 69.4%). ¹HNMR (300 MHz, DMSO-d₆) δ 7.34-7.06 (m, 5H), 4.80 (t, J=6.3 Hz, 2H), 3.41 (t, J=6.3 Hz, 2H), 2.33 (s, 3H), 1.36 (s, 9H).

Synthesis of tert-butyl (R)-3-(2-((tert-butoxycarbonyl)amino)-2-(o-tolyl)ethoxy)propanoate: Into a 40 mL vial were added tert-butyl (R)-(2-hydroxy-1-(o-tolyl)ethyl)carbamate (370 mg, 1.5 mmol, 1.0 eq), t-BuOH (5 mL), tert-butyl prop-2-enoate (3.8 g, 29.6 mmol, 20.1 eq), Cs₂CO₃ (479 mg, 1.5 mmol, 1.0 eq). The resulting mixture was stirred overnight at 45° C. The reaction was diluted with water (300 mL). The resulting solution was extracted with ethyl acetate (3×50 mL) and the organic layers combined. The resulting mixture was washed with brine (300 mL). The mixture was dried over anhydrous Na₂SO₄. After filtration, the resulting mixture was concentrated under vacuum. The crude residue was applied onto a silica gel column and eluted with ethyl acetate/petroleum ether=10:1 to give tert-butyl (R)-3-(2-((tert-butoxycarbonyl)amino)-2-(o-tolyl)ethoxy)propanoate as a colorless oil (530 mg, 94.8%). LC-MS (ESI, m/z) M+1: 380.

Synthesis of (R)-3-(2-amino-2-(o-tolyl)ethoxy)propanoic acid hydrochloride: Into a 40 mL vial were added tert-butyl (R)-3-(2-((tert-butoxycarbonyl)amino)-2-(o-tolyl)ethoxy)propanoate (500 mg, 1.3 mmol, 1.0 eq) in HCl (gas) in 1,4-dioxane (4 M, 5 mL) was stirred for 3 hours at 25° C. The resulting mixture was concentrated under vacuum to give (R)-3-(2-amino-2-(o-tolyl)ethoxy)propanoic acid hydrochloride as a white solid (340 mg, 99.4%). LC-MS (ESI, m/z) M+1: 224.

Synthesis of (R)-3-(o-tolyl)-1,4-oxazepan-5-one: Into a 40 mL vial were added (R)-3-(2-amino-2-(o-tolyl)ethoxy)propanoic acid hydrochloride (340 mg, 1.3 mmol, 1.0 eq), CH₂Cl₂ (5 mL), EDCI (301 mg, 1.6 mmol, 1.2 eq), HOBT (212 mg, 1.6 mmol, 1.2 eq), TEA (265 mg, 2.6 mmol, 2.0 eq). The resulting mixture was stirred overnight at 25° C. The resulting mixture was concentrated under vacuum. The crude residue was applied onto a silica gel column and eluted with ethyl dichloromethane/methanol=10:1 to give (R)-3-(o-tolyl)-1,4-oxazepan-5-one as a white solid (200 mg, 74.4%). LC-MS (ESI, m/z) M+1: 206. ¹HNMR (300 MHz, DMSO-d₆) δ 7.43 (s, 1H), 7.35-7.26 (m, 1H), 7.20 (d, J=2.4 Hz, 3H), 4.90 (dt, J=7.5, 2.7 Hz, 1H), 3.91 (ddd, J=12.3, 5.7, 2.7 Hz, 1H), 3.80-3.66 (m, 2H), 3.70-3.58 (m, 1H), 3.04 (ddd, J=15.3, 10.8, 2.7 Hz, 1H), 2.45-2.37 (m, 1H), 2.35 (s, 3H).

Synthesis of (3R)-3-(2-methylphenyl)-1,4-oxazepane: To a stirred solution of (R)-3-(o-tolyl)-1,4-oxazepan-5-one (200 mg, 1.0 mmol, 1.0 eq) in THE (10 mL) was added LAlH₄ (111 mg, 3.0 mmol, 3.00 eq) in portions at 0° C. under nitrogen atmosphere. The resulting mixture was stirred overnight at 25° C. The reaction was quenched with Na₂SO₄·10H₂O at 0° C. and the resulting mixture was filtered. The resulting mixture was concentrated under vacuum. The crude residue was applied onto a silica gel column and eluted with ethyl dichloromethane/methanol=10:1 to give (3R)-3-(2-methylphenyl)-1,4-oxazepane as a light yellow oil (43 mg, 23.1%). ¹HNMR (300 MHz, DMSO-d₆) δ 7.52-7.44 (m, 1H), 7.28-7.12 (m, 3H), 4.17 (dd, J=9.6, 3.3 Hz, 1H), 4.04 (dt, J=12.3, 6.2 Hz, 1H), 3.97-3.81 (m, 2H), 3.48 (dd, J=12.3, 9.6 Hz, 1H), 3.27 (dt, J=13.2, 5.1 Hz, 1H), 3.03 (dt, J=13.2, 7.2 Hz, 1H), 2.40 (s, 3H), 2.03 (ddt, J=12.9, 6.6, 3.6 Hz, 3H).

Synthesis of N-((4-((((1r,4r)-4-hydroxy-4-methylcyclohexyl)methyl)amino)-3-nitrophenyl)sulfonyl)-4-(2-((R)-3-(o-tolyl)-1,4-oxazepan-4-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial, were placed (R)-3-(o-tolyl)-1,4-oxazepane (19 mg, 0.09 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (87 mg, 0.09 mmol, 1.0 eq), methanol (2 mL), ZnCl₂ (25 mg, 0.18 mmol, 2.0 eq), NaBH₃CN (29 mg, 0.46 mmol, 5.0 eq). The reaction mixture was stirred overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1) to give N-((4-((((1r,4r)-4-hydroxy-4-methylcyclohexyl)methyl)amino)-3-nitrophenyl)sulfonyl)-4-(2-((R)-3-(o-tolyl)-1,4-oxazepan-4-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (80 mg, 76.3%). LC-MS (ESI, m/z) M+1: 1119.

Synthesis of N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide hydrochloride: Into a 40 mL vial, were placed N-((4-((((1r,4r)-4-hydroxy-4-methylcyclohexyl)methyl)amino)-3-nitrophenyl)sulfonyl)-4-(2-((R)-3-(o-tolyl)-1,4-oxazepan-4-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (80 mg, 0.07 mmol, 1.0 eq), TBAF-THF (5 mL, 1 M), ethylenediamine (85 mg, 1.4 mmol, 20.0 eq). The reaction mixture was stirred for 5 hours at 70° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=11:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc.HCl (1 drop)). Finally, N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(o-tolyl)-1,4-oxazepan-4-yl)-7-azaspiro[3.5]nonan-7-yl)benzamide hydrochloride was obtained as a yellow solid (15 mg, 20.4%). LC-MS (ESI, m/z) M-HCl+1: 989. ¹HNMR (300 MHz, DMSO-d₆) δ 12.75 (s, 1H), 11.26 (s, 1H), 9.81 (s, 1H), 8.37 (d, J=2.4 Hz, 2H), 7.80 (d, J=9.0 Hz, 2H), 7.68 (s, 1H), 7.14 (dd, J=3.3, 2.4 Hz, 2H), 6.99 (t, J=8.7 Hz, 2H), 6.48 (d, J=11.1 Hz, 3H), 6.06 (dd, J=3.3, 1.8 Hz, 2H), 4.56 (s, 2H), 4.26 (s, 6H), 3.78 (d, J=16.5 Hz, 6H), 3.36 (d, J=10.8 Hz, 4H), 3.26 (s, 3H), 3.12 (s, 2H), 2.37 (s, 5H), 2.18-1.91 (m, 5H), 1.68 (d, J=13.2 Hz, 3H), 1.50 (s, 4H), 1.50-1.32 (m, 7H).

The compounds below are prepared by methods substantially identical, similar, or analogous to those disclosed in above Schemes and Examples:

| Compound | Chemical Name | m/z(MH⁺) |
|---|---|---|
| A | N-((4-((((1r,4r)-4-hydroxy-4-methylcyclohexyl)methyl)amino)-3- | 989 |
| | nitrophenyl)sulfonyl)-4-((2S,4S,6R)-6-methyl-2-((R)-3-(o-tolyl)morpholino)- | |
| | 7-azaspiro[3.5]nonan-7-yl)-2-((5aS,9aR)-6,7,9,9a-tetrahydro-1H- | |
| | pyrano[3,4-b]pyrrolo[3′,2′:5,6]pyrido[3,2-e][1,4]oxazin-5(5aH)-yl)benzamide, | |
| B | N-((4-((((1r,4r)-4-hydroxy-4-methylcyclohexyl)methyl)amino)-3- | 989 |
| | nitrophenyl)sulfonyl)-4-((2R,4R,6R)-6-methyl-2-((R)-3-(o-tolyl)morpholino)- | |
| | 7-azaspiro[3.5]nonan-7-yl)-2-((5aS,9aR)-6,7,9,9a-tetrahydro-1H- | |
| | pyrano[3,4-b]pyrrolo[3′,2′:5,6]pyrido[3,2-e][1,4]oxazin-5(5aH)-yl)benzamide, | |
| C | N-((4-((((1r,4r)-4-hydroxy-4-methylcyclohexyl)methyl)amino)-3- | 989 |
| | nitrophenyl)sulfonyl)-4-((2R,4S,6S)-6-methyl-2-((R)-3-(o-tolyl)morpholino)- | |
| | 7-azaspiro[3.5]nonan-7-yl)-2-((5aS,9aR)-6,7,9,9a-tetrahydro-1H- | |
| | pyrano[3,4-b]pyrrolo[3′,2′:5,6]pyrido[3,2-e][1,4]oxazin-5(5aH)-yl)benzamide, | |
| D | N-((4-((((1r,4r)-4-hydroxy-4-methylcyclohexyl)methyl)amino)-3- | 989 |
| | nitrophenyl)sulfonyl)-4-((2S,4R,6S)-6-methyl-2-((R)-3-(o-tolyl)morpholino)- | |
| | 7-azaspiro[3.5]nonan-7-yl)-2-((5aS,9aR)-6,7,9,9a-tetrahydro-1H- | |
| | pyrano[3,4-b]pyrrolo[3′,2′:5,6]pyrido[3,2-e][1,4]oxazin-5(5aH)-yl)benzamide, | |

### Biological Example 1: Bcl-2 Competition Binding (Fluorescence Polarization) Assay

The fluorescence-labeled 23 amino acid peptide BH3 was purchased from CalBiochem (NLWAAQRYGRELRRMSDKFVD). An unbound Fluorescein labeled BH3 peptide emits random light with respect to the plane of polarization plane of excited light, resulting in a lower polarization degree (mP) value. When the peptide is bound to Bcl-2, the complex tumble slower and the emitted light can have a higher level of polarization, resulting in a higher mP value. This binding assay was performed in 96-well plate and with each assay contained 15 and 30 nM of labeled peptide and purified Bcl-2 protein (purchased from R&D Systems, Inc). The assay buffer contained 20 mM Hepes (pH 7.0), 50 mM KCl, 5 mM MgCl₂, 20 mM Na₂MoO₄, 0.1 mg/mi Bovine Gamma Globulin and 0.01% NP40. Compounds were diluted in DMS0 and added to the final assay with concentration range from 20 μM to 2 nM. The polarization degree (mP) value was determined by BioTek Synergy II with background subtraction after 3 hours of incubation at room temperature. IC₅₀ was calculated using Prism software with sigmoidal dose-response curve fitting. ABT-737 was used as reference compound. Such assays, carried out with a range of doses of test compounds, allowed the determination of an approximate IC₅₀ value. Although the inhibitory properties of the compounds of the present invention vary with structural change as expected, the activity generally exhibited by these agents was in the range of IC₅₀=0.1-1000 nM. The following table lists the IC₅₀ values of certain compounds of the invention.

| Example | Bcl-2 IC₅₀ (nM) |
|---|---|
| ABT-199 | 0.42 |
| Example 1 | 0.10 |
| Example 2 | 0.22 |
| Example 3-0A | 0.16 |
| Example 3-0B | 0.13 |
| Example 4 | 0.183 |
| Example 5 | 0.067 |
| Example 6 | 0.083 |
| Example 7 | 0.066 |
| Example 8 | 0.066 |
| Example 12 | 0.161 |
| Example 13 | 2.42 |
| Example 14 | 0.74 |
| Example 15 | 0.2 |
| Example 16 | 0.09 |
| Example 17 | 0.225 |
| Example 18 | 0.42 |
| Example 19 | 0.10 |
| Example 20 | 0.299 |
| Example 21 | 0.239 |
| Example 22 | 0.10 |
| Example 23 | 0.587 |
| Example 24 | 0.42 |
| Example 25 | 14.8 |
| Example 26 | 0.70 |
| Example 27 | 1.2 |
| Example 28 | 31.2 |
| Example 29 | 1.39 |
| Example 30 | 15.8 |
| Example 31 | 30.70 |
| Example 32 | 15.8 |
| Example 33 | 1.42 |
| Example 34 | 3.50 |
| Example 35 | 0.58 |
| Example 36 | 0.31 |
| Example 37 | 0.358 |
| Example 38 | 0.202 |
| Example 39 | 0.19 |
| Example 40 | 0.20 |
| Example 41 | 0.18 |
| Example 42 | 0.217 |
| Example 43 | 0.28 |
| Example 44 | 0.179 |
| Example 45 | 0.198 |
| Example 46 | 0.398 |
| Example 47 | 0.307 |
| Example 50 | 0.265 |
| Example 51 | 1.080 |
| Example 54 | 0.586 |
| Example 55 | 0.608 |
| Example 58 | 0.324 |
| Example 59 | 0.101 |
| Example 60 | 0.341 |
| Example 61 | 0.707 |
| Example 62 | 0.41 |
| Example 63 | 0.314 |
| Example 64 | 0.341 |
| Example 65 | 0.425 |
| Example 66 | 0.564 |
| Example 67 | 0.229 |
| Example 68 | 0.303 |

### Biological Example 2: In vitro Anti-proliferation Assay in BCL-2-dependent acute lymphoblastic leukemia (ALL) cell line RS4; 11 with G101V mutation

Cell antiproliferation was assayed by PerkinElmer ATPlite™ Luminescence Assay System. Briefly, the various test cancer cell lines were plated at a density of about 1×10⁴ cells per well in Costar 96-well plates, and were incubated with different concentrations of compounds for about 72 hours in medium supplemented with 5% FBS or 10% normal human serum (NHS). One lyophilized substrate solution vial was then reconstituted by adding 5 mL of substrate buffer solution, and was agitated gently until the solution was homogeneous. About 50 μL of mammalian cell lysis solution was added to 100 μL of cell suspension per well of a microplate, and the plate was shaken for about five minutes in an orbital shaker at ˜700 rpm. This procedure was used to lyse the cells and to stabilize the ATP. Next, 50 μL substrate solution was added to the wells and microplate was shaken for five minutes in an orbital shaker at ˜700 rpm. Finally, the luminescence was measured by a PerkinElmer TopCount® Microplate Scintillation Counter. Such assays, carried out with a range of doses of test compounds, allowed the determination of the cellular anti-antiproliferative IC₅₀ of the compounds of the present invention.

The following table lists the IC₅₀ values of certain compounds of the invention.

| Example | RS4; 11-G101V IC50 (nM) |
|---|---|
| ABT-199 | 1200 |
| Example 1 | 5.1 |
| Example 2 | 66.7 |
| Example 3-0A | 5.6 |
| Example 3-0B | 25.0 |
| Example 4 | 28.3 |
| Example 5 | 9.5 |
| Example 6 | 29.0 |
| Example 7 | 11.5 |
| Example 8 | 9.1 |
| Example 17 | 99.5 |
| Example 19 | 71.8 |
| Example 20 | 60.4 |
| Example 36 | 56.4 |
| Example 37 | 15.2 |
| Example 38 | 13.6 |
| Example 39 | 49.2 |
| Example 40 | 34.7 |
| Example 41 | 14.7 |
| Example 42 | 29.2 |
| Example 43 | 86.4 |
| Example 44 | 98.3 |
| Example 45 | 23.8 |
| Example 46 | 58.2 |
| Example 47 | 288 |
| Example 50 | 71.8 |
| Example 51 | 359 |
| Example 54 | >1000 |
| Example 55 | 392 |
| Example 58 | 188 |
| Example 59 | 124 |
| Example 60 | 62.4 |
| Example 61 | 130 |
| Example 62 | 121 |
| Example 63 | 49.6 |
| Example 64 | 62.4 |
| Example 65 | >1000 |
| Example 66 | 296 |
| Example 67 | 50.6 |
| Example 68 | 40.7 |

### Biological Example 3: Mice PK Study

The pharmacokinetics of compounds were evaluated in CD-1 mouse via Intravenous and Oral Administration. The IV dose was administered as a slow bolus in the Jugular vein, and oral doses were administered by gavage. The formulation for IV dosing was 5% DMSO in 20% HPBCD in water, and the PO formulation was 2.5% DMSO, 10% EtOH, 20% Cremphor EL, 67.5% D5W. The PK time point for the IV arm was 5, 15, 30 min, 1, 2, 4, 6, 8, 12, 24 hours post dose, and for PO arm was 15, 30 min, 1, 2, 4, 6, 8, 12, 24 hours post dose. Approximately 0.03 mL blood was collected at each time point. Blood of each sample was transferred into plastic micro centrifuge tubes containing EDTA-K2 and collect plasma within 15 min by centrifugation at 4000 g for 5 minutes in a 4° C. centrifuge. Plasma samples were stored in polypropylene tubes. The samples were stored in a freezer at −75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary.

### Biological Example 4: In Vivo Xenograft Studies

Compound of Example 3 is selected for in vivo studies in the BCL-2-dependent acute lymphoblastic leukemia (ALL) RS4; 11 xenograft model. The CB.17 SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach ˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

What is claimed is:

1. A compound of Formula (I), or an N-oxide thereof, or a pharmaceutically acceptable salt, solvate, polymorph, tautomer, stereoisomer, an isotopic form, or a prodrug of said compound of Formula (I) or N-oxide thereof: 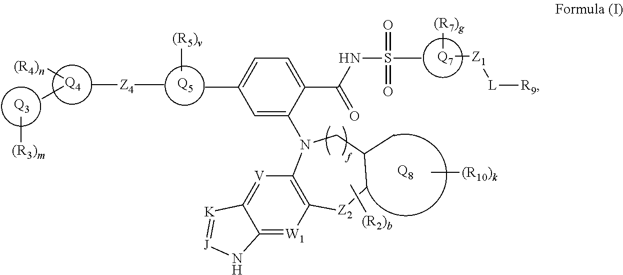
wherein
each of W₁, V, K, J, independently, is C(Rₐ) or N;
each of Q₃, Q₄, Q₅, Q₇, and Q₈, independently, is cycloalkyl, cycloalkenyl, heterocycloalkyl, heterocycloalkenyl, spiroheterocyclyl, fused heterocyclyl, bridged heterocyclyl, aryl, or heteroaryl, wherein one or more (e.g., both) border ring atom(s) between ring Q₈ and the ring with Z₂ can be carbon or heteroatom(s);
each of R₁, R₂, R₃, R₄, R₅, R₆, R₇, R₉, and R₁₀, independently, is H, D, alkyl, spiroalkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiroheterocyclyl, fused heterocyclyl, bridged heterocyclyl, aryl, or heteroaryl is optionally substituted with one or more R_{d};
two of R₁ group, taken together with the atom to which they are attached, may optionally form a cycloalkyl, heterocycloalkyl, aryl, or heteroaryl, in which said cycloalkyl, heterocycloalkyl, aryl, or heteroaryl of R₁, is optionally substituted with one or more R_{d};
two of R₂ group, taken together with the atom to which they are attached, may optionally form a cycloalkyl, heterocycloalkyl, aryl, or heteroaryl, in which said cycloalkyl, heterocycloalkyl, aryl, or heteroaryl of R₂, is optionally substituted with one or more R_{d};
two of R₃ group, taken together with the atom to which they are attached, may optionally form a cycloalkyl, heterocycloalkyl, aryl, or heteroaryl, in which said cycloalkyl, heterocycloalkyl, aryl, or heteroaryl of R₃, is optionally substituted with one or more R_{d};
two of R₄ group, taken together with the atom to which they are attached, may optionally form a cycloalkyl, heterocycloalkyl, aryl, or heteroaryl, in which said cycloalkyl, heterocycloalkyl, aryl, or heteroaryl of R₄, is optionally substituted with one or more R_{d};
two of R₅ group, taken together with the atom to which they are attached, may optionally form a cycloalkyl, heterocycloalkyl, aryl, or heteroaryl, in which said cycloalkyl, heterocycloalkyl, aryl, or heteroaryl of R₅, is optionally substituted with one or more R_{d};
two of R₆ group, taken together with the atom to which they are attached, may optionally form a cycloalkyl, heterocycloalkyl, aryl, or heteroaryl, in which said cycloalkyl, heterocycloalkyl, aryl, or heteroaryl of R₆, is optionally substituted with one or more R_{d};
two of R₇ group, taken together with the atom to which they are attached, may optionally form a cycloalkyl, heterocycloalkyl, aryl, or heteroaryl, in which said cycloalkyl, heterocycloalkyl, aryl, or heteroaryl of R₇, is optionally substituted with one or more R_{d};
two of R₁₀ group, taken together with the atom to which they are attached, may optionally form a cycloalkyl, heterocycloalkyl, aryl, or heteroaryl, in which said cycloalkyl, heterocycloalkyl, aryl, or heteroaryl of R₈, is optionally substituted with one or more R_{d};
R₃ and R₄ group, taken together with the atom to which they are attached, may optionally form a cycloalkyl, heterocycloalkyl, aryl, or heteroaryl, in which said cycloalkyl, heterocycloalkyl, aryl, or heteroaryl of R₃ or R₄, is optionally substituted with one or more R_{d};
R₄ and R₅ group, taken together with the atom to which they are attached, may optionally form a cycloalkyl, heterocycloalkyl, aryl, or heteroaryl, in which said cycloalkyl, heterocycloalkyl, aryl, or heteroaryl of R₄ or R₅, is optionally substituted with one or more R_{d};
R₅ and R₆ group, taken together with the atom to which they are attached, may optionally form a cycloalkyl, heterocycloalkyl, aryl, or heteroaryl, in which said cycloalkyl, heterocycloalkyl, aryl, or heteroaryl of R₅ or R₆, is optionally substituted with one or more R_{d};
R₂ and R₁₀ group, taken together with the atom to which they are attached, may optionally form a cycloalkyl, heterocycloalkyl, aryl, or heteroaryl, in which said cycloalkyl, heterocycloalkyl, aryl, or heteroaryl of R₂ and R₁₀, is optionally substituted with one or more R_{d};
R₇ and —Z₁-L-R₉ group, taken together with the atom to which they are attached, may optionally form a cycloalkyl, cycloalkenyl, heterocycloalkyl, heterocycloalkenyl, spiroheterocyclyl, fused heterocyclyl, bridged heterocyclyl, aryl, or heteroaryl, in which said cycloalkyl, cycloalkenyl, heterocycloalkyl, heterocycloalkenyl, spiroheterocyclyl, fused heterocyclyl, bridged heterocyclyl, aryl, or heteroaryl of R₇, is optionally substituted with one or more R_{d};
each of Z₁, and Z₂, independently, is a bond, (CRₐR_{b})ₚ, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, (CRₐR_{b})ₚC(O)N(Rₐ)(CRₐR_{b})_{q}, a bivalent alkenyl group, or a bivalent alkynyl group;
Z₄ is a bond or a cycloalkyl, cycloalkenyl, heterocycloalkyl, heterocycloalkenyl, spiroheterocyclyl, fused heterocyclyl, bridged heterocyclyl, aryl, or heteroaryl, in which said cycloalkyl, cycloalkenyl, heterocycloalkyl, heterocycloalkenyl, spiroheterocyclyl, fused heterocyclyl, bridged heterocyclyl, aryl, or heteroaryl is optionally substituted with one or more R_{d};
L is bond, alkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, alkenyl, alkynyl, cycloalkyl, cycloalkenyl, heterocycloalkyl, heterocycloalkenyl, spiroheterocyclyl, fused heterocyclyl, bridged heterocyclyl, aryl, or heteroaryl is optionally substituted with one or more R_{d};
each of Rₐ, R_{b}, R_{c}, and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, alkoxy, alkoxyalkyl, cycloalkyl, cycloalkenyl, heterocycloalkyl, heterocycloalkenyl, spiroheterocyclyl, fused heterocyclyl, bridged heterocyclyl, aryl, heteroaryl is optionally substituted with one or more Rₑ;
each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl, in which said alkyl, alkoxy, alkoxyalkyl, cycloalkyl, cycloalkenyl, heterocycloalkyl, heterocycloalkenyl, spiroheterocyclyl, fused heterocyclyl, bridged heterocyclyl, aryl, heteroaryl is optionally substituted with one or more R_{f}; and,
each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl;
Rₐ and R_{b}, groups, taken together with the atom to which they are attached, may optionally form a cycloalkyl, heterocycloalkyl, aryl, or heteroaryl, in which said cycloalkyl, heterocycloalkyl, aryl, or heteroaryl is optionally substituted with one or more Rₑ;
R_{b} and R_{c} groups, taken together with the atom to which they are attached, may optionally form a cycloalkyl, heterocycloalkyl, aryl, or heteroaryl, in which said cycloalkyl, heterocycloalkyl, aryl, or heteroaryl of R_{b} and R_{c}, is optionally substituted with one or more Rₑ;
two of R_{d} group, taken together with the atom to which they are attached, may optionally form a cycloalkyl, heterocycloalkyl, aryl, or heteroaryl, in which said cycloalkyl, heterocycloalkyl, aryl, or heteroaryl of R_{d}, is optionally substituted with one or more Rₑ;
two of Rₑ groups, taken together with the atom to which they are attached, may optionally form a cycloalkyl, heterocycloalkyl, aryl, or heteroaryl, in which said cycloalkyl, heterocycloalkyl, aryl, or heteroaryl of Rₑ is optionally substituted with one or more R_{f}; and
two of R_{f} groups, taken together with the atom to which they are attached, may optionally form a cycloalkyl, heterocycloalkyl, aryl, or heteroaryl, in which said cycloalkyl, heterocycloalkyl, aryl, or heteroaryl of R_{f} is optionally substituted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiroheterocyclyl, fused heterocyclyl, bridged heterocyclyl, aryl, or heteroaryl; and
each of a, b, m, n, j, k, v and g is, independently, 0, 1, 2, 3, 4, 5, 6, 7, or 8;
f is 0, 1, or 2; and
each of p, and q is, independently, 0, 1, 2, 3, 4, or 5.

2. The compound according to claim 1 or an N-oxide thereof, or a pharmaceutically acceptable salt, solvate, polymorph, tautomer, stereoisomer, an isotopic form, or a prodrug thereof, wherein the compound is represented by Formula (II):

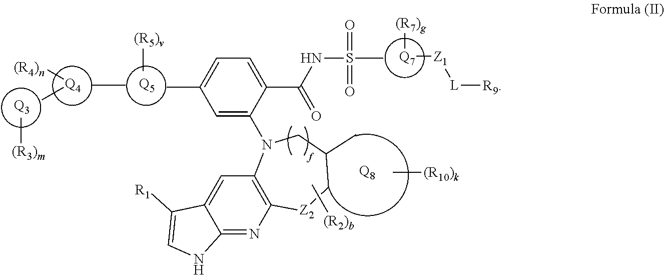

3. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (III): 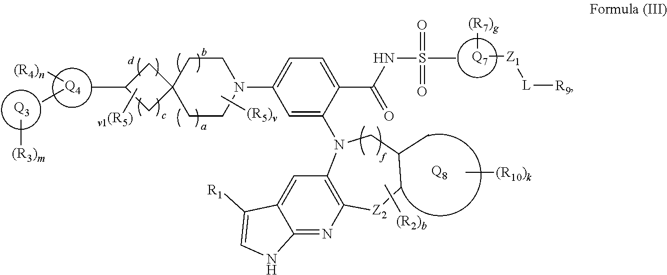
wherein each of a, b, c, and d, independently, is 0, 1, or 2; and wherein v1 and v2 are both integers including 0, and v1+v2=v.

4. The compound according to claim 3 or an N-oxide thereof, or a pharmaceutically acceptable salt, solvate, polymorph, tautomer, stereoisomer, an isotopic form, or a prodrug thereof, wherein the compound is represented by Formula (IV): 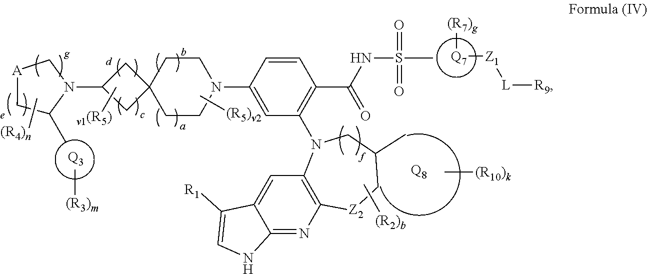
wherein each of e and g, independently is 0, 1, or 2; and A is O, S, SO₂, N(Rₐ), or C(RₐR_{b}).

5. The compound according to claim 4 or an N-oxide thereof, or a pharmaceutically acceptable salt, solvate, polymorph, tautomer, stereoisomer, an isotopic form, or a prodrug thereof, wherein the compound is represented by Formula (IV-A):

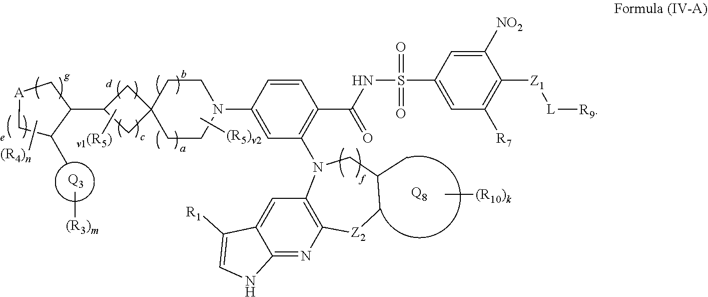

6. The compound according to claim 4 or an N-oxide thereof, or a pharmaceutically acceptable salt, solvate, polymorph, tautomer, stereoisomer, an isotopic form, or a prodrug thereof, wherein the compound is represented by Formula (IV-B): 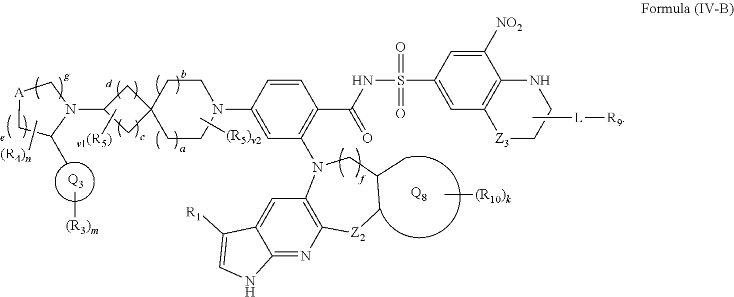
wherein Z₃ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, or OC(O)N(H)(CH₂)ₚ+₁N(H)(CH₂)_{q}.

7. A pharmaceutical composition comprising a compound of any one of Formulae (I)-(IV-B), or an N-oxide thereof as defined in claims 1-6, or a pharmaceutically acceptable salt, solvate, polymorph, tautomer, stereoisomer, an isotopic form, or a prodrug of said compound of any one of Formulae (I)-(IV-B) or N-oxide thereof, and a pharmaceutically acceptable diluent or carrier.

8. A method of treating a neoplastic disease, an autoimmune disease, or a neorodegenerative disease, comprising administering to a subject in need thereof an effective amount of a compound of any one of Formulas (I)-(IV-B) or an N-oxide thereof as defined in claims 1-6, or a pharmaceutically acceptable salt, solvate, polymorph, tautomer, stereoisomer, an isotopic form, or a prodrug of said compound of any one of Formulae (I)-(IV-B) or N-oxide thereof.
